(19) United States
(12) Patent Application Publication
(10) Pub. No.: US 2024/0092776 A1
(43) Pub. Date: Mar. 21, 2024

(54) HETEROCYCLIC COMPOUND, ORGANIC LIGHT-EMITTING DEVICE COMPRISING SAME

(71) Applicant: LT MATERIALS CO., LTD., Yongin-City (KR)

(72) Inventors: Hyung-Jin LEE, Yongin-City (KR); Won-Jang JEONG, Yongin-City (KR); Dong-Jun KIM, Yongin-City (KR); Dae-Hyuk CHOI, Yongin-City (KR)

(73) Assignee: LT MATERIALS CO., LTD., Yongin-City (KR)

(21) Appl. No.: 18/212,995

(22) Filed: Jun. 22, 2023

(30) Foreign Application Priority Data
Aug. 9, 2022 (KR) 10-2022-0099445

(51) Int. Cl. C07D 471/04; C07D 401/10; C07D 405/12; C09K 11/06; H10K 50/11; H10K 50/17; H10K 50/18; H10K 50/805; H10K 85/60

(52) U.S. Cl. CPC C07D 471/04; C07D 401/10; C07D 405/12; C09K 11/06; H10K 50/11; H10K 50/171; H10K 50/181; H10K 50/805; H10K 85/6572; C07B 2200/05

(57) ABSTRACT

Disclosed (or Provided) are a heterocyclic compound and an organic light emitting device including the same.

### CROSS-REFERENCE TO RELATED APPLICATIONS

This application claims priority to and the benefit of Korean Patent Application No. 10-2022-0099445 filed in the Korean Intellectual Property Office on Aug. 9, 2022, the entire contents of which are incorporated herein by reference.

### TECHNICAL FIELD

The present invention relates to a heterocyclic compound and an organic light emitting device including the same.

### BACKGROUND ART

An electroluminescence device is a kind of self-emitting type display device, and has an advantage in that the viewing angle is wide, the contrast is excellent, and the response speed is fast.

An organic light emitting device is composed of a structure in which an organic thin film is disposed between two electrodes. When a voltage is applied to an organic light emitting device having such a structure, electrons and holes injected from the two electrodes are combined with each other in the organic thin film to make a pair, and then, the paired electrons and holes emit light while being annihilated. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may serve as a hole injection layer, a hole transport layer, an electron blocking layer, a hole blocking layer, an electron transport layer, an electron injection layer, a charge generation layer, and the like.

In order to improve the performance, efficiency and service life of the organic light emitting device, there is a continuous need for developing a material for an organic thin film.

### RELATED ART DOCUMENTS

### Patent Document

(Patent Document 1) U.S. Pat. No. 4,356,429

### SUMMARY OF THE INVENTION

The present invention has been made in an effort to provide a heterocyclic compound and an organic light emitting device including the same.

An exemplary embodiment of the present invention provides a heterocyclic compound represented by the following Chemical Formula 1.

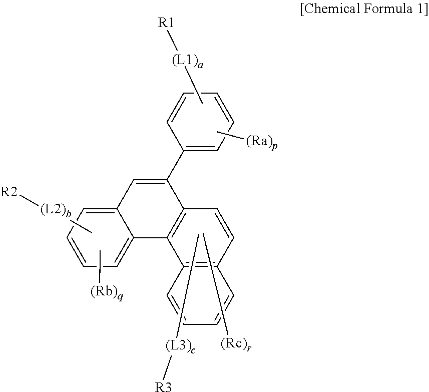

In Chemical Formula 1,
L1 to L3 are the same as or different from each other, and are each independently a direct bond; a substituted or unsubstituted C6 to C60 arylene group; or a substituted or unsubstituted C2 to C60 heteroarylene group,
R1 to R3 are the same as or different from each other, and are each independently hydrogen; deuterium; a substituted or unsubstituted amine group; a substituted or unsubstituted C6 to C60 aryl group; a substituted or unsubstituted C2 to C60 heteroaryl group; or —P(═O)RR′,
at least one of R1 to R3 is a substituted or unsubstituted amine group, and another one of R1 to R3 is a substituted or unsubstituted C2 to C60 heteroaryl group; or —P(═O)RR′,
R and R′ are the same as or different from each other, and are each independently hydrogen; deuterium; —CN; a substituted or unsubstituted C6 to C60 aryl group; or a substituted or unsubstituted C2 to C60 heteroaryl group,
Ra, Rb and Rc are the same as or different from each other, and are each independently hydrogen; deuterium; a halogen group; —CN; a substituted or unsubstituted C1 to C60 alkyl group; a substituted or unsubstituted C6 to C60 aryl group; or a substituted or unsubstituted C2 to C60 heteroaryl group,
a, b and c are an integer from 0 to 4, and when a, b and c are 2 or higher, substituents in the parenthesis are the same as or different from each other,
p is an integer from 0 to 4, and when p is 2 or higher, substituents in the parenthesis are the same as or different from each other,
q is an integer from 0 to 3, and when q is 2 or higher, substituents in the parenthesis are the same as or different from each other, and
r is an integer from 0 to 5, and when r is 2 or higher, substituents in the parenthesis are the same as or different from each other.

Another exemplary embodiment provides an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

The heterocyclic compound described in the present specification may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a charge generation layer and an electron transport layer of an organic light emitting device.

Specifically, one or two or more of the heterocyclic compounds represented by Chemical Formula 1 can be used, and the heterocyclic compounds represented by Chemical Formula 1 can be used as materials for the charge generation layer and the electron transport layer. In particular, the heterocyclic compound can be used as a material for the charge generation layer and the electron transport layer of an organic light emitting device by introducing various substituents and changing the binding position of the substituent to adjust the bandgap.

The heterocyclic compound of the present invention has an advantage in that structural stability is better than a structure in which an amine-based substituent is not substituted, a mono-substituted structure or a tri-substituted structure and hole mobility is also higher than that of a mono- or tri-substituted structure by having a di-substituted structure in which (1) an amine-based substituent and (2) a heterocyclic group or phosphine oxide group are introduced into the core structure thereof and simultaneously binding the above-described substituents in (1) and (2) to specific positions.

In particular, when the heterocyclic compound of the present invention is used in the charge generation layer or electron transport layer of an organic light emitting device, it is possible to obtain an effect in which the driving voltage of the device is lowered, the efficiency of the device is also increased, and the service life of the device is extended.

### BRIEF DESCRIPTION OF THE DRAWINGS

FIGS. 1 to 4 are views each schematically illustrating a stacking structure of an organic light emitting device according to an exemplary embodiment of the present application.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification,

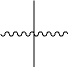

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may also be interpreted as an element which has the same number of protons, but different number of neutrons.

In an exemplary embodiment of the present application, when the total number of substituents of a basic compound is defined as T1 and the number of specific substituents among the substituents is defined as T2, the content 1% of the specific substituent may be defined as T2/T1×100=T %.

That is, in an example, the deuterium content of 20% in a phenyl group represented by

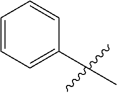 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

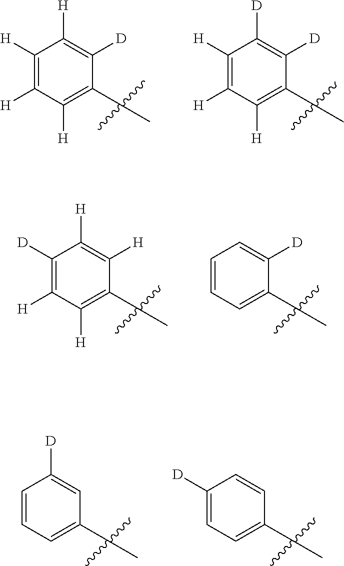 [SMILES: *c1ccccc1[2H] ; *c1cccc([2H])c1[2H] ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

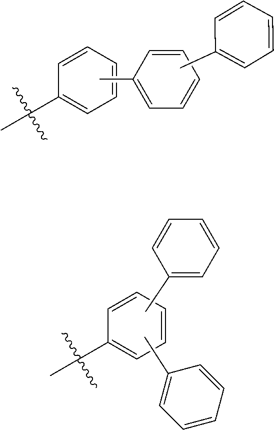

In the present specification, the fluorenyl group may be substituted, and adjacent substituents may be bonded to each other to form a ring.

When the fluorenyl group is substituted, the substituent may be

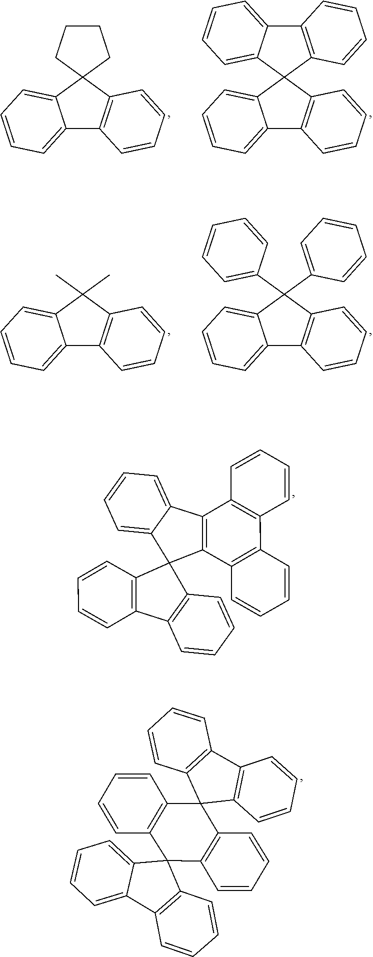

and the like, but is not limited thereto.

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydrodibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

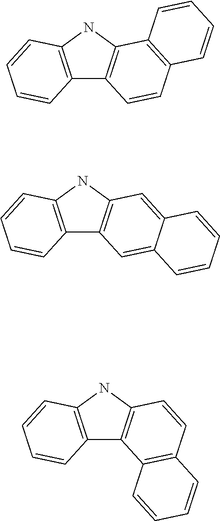 [SMILES: c1ccc2c(c1)ccc1c3ccccc3[nH]c21 ; c1ccc2cc3c(cc2c1)[nH]c1ccccc13 ; c1ccc2c(c1)ccc1[nH]c3ccccc3c12]

In the present specification, a dibenzocarbazole group may be any one of the following structures.

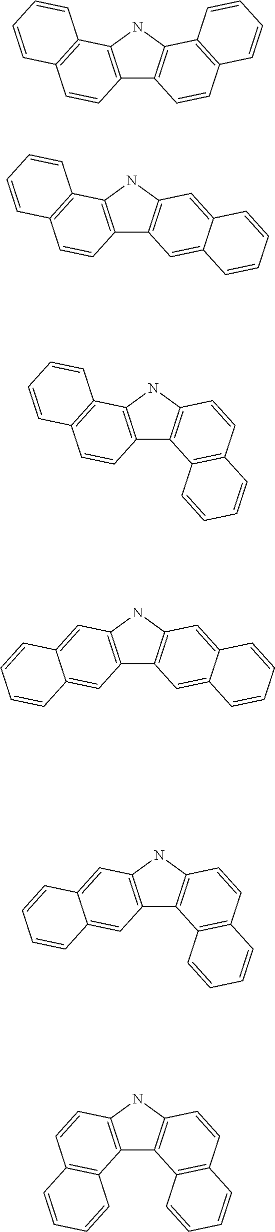 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, a naphthobenzofuran group may be any one of the following structures.

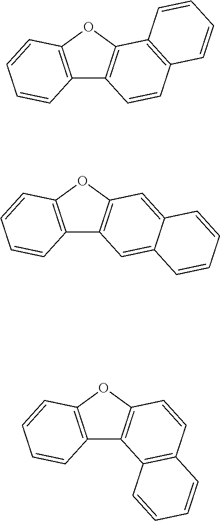 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

In the present specification, a naphthobenzothiophene group may be any one of the following structures.

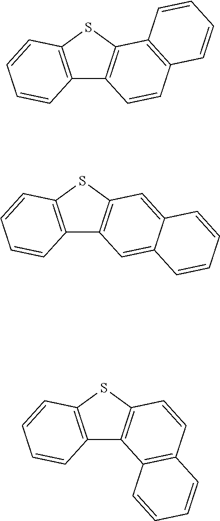 [SMILES: c1ccc2c(c1)ccc1c3ccccc3sc21 ; c1ccc2cc3c(cc2c1)sc1ccccc13 ; c1ccc2c(c1)ccc1sc3ccccc3c12]

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

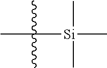 [SMILES: *[Si](C)(C)C]

(a trimethylsilyl group),

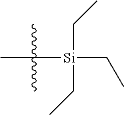 [SMILES: *[Si](CC)(CC)CC]

(a triethylsilyl group),

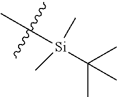 [SMILES: *[Si](C)(C)C(C)(C)C]

(a t-butyldimethylsilyl group),

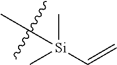 [SMILES: *[Si](C)(C)C=C]

(a vinyldimethylsilyl group),

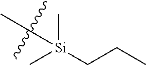 [SMILES: *[Si](C)(C)CCC]

(a propyldimethylsilyl group),

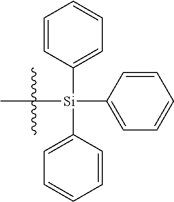 [SMILES: *[Si](c1ccccc1)(c1ccccc1)c1ccccc1]

(a triphenylsilyl group),

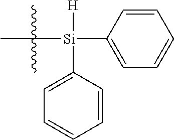 [SMILES: *[SiH](c1ccccc1)c1ccccc1]

(a diphenylsilyl group),

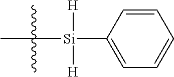 [SMILES: *[SiH2]c1ccccc1]

(a phenylsilyl group) and the like, but are not limited thereto.

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112) (R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

In an exemplary embodiment of the present application, provided is the compound represented by Chemical Formula 1.

In an exemplary embodiment of the present application, a group not represented by a substituent; or a group represented by hydrogen may mean being all substitutable with deuterium. That is, it may be shown that hydrogen; or deuterium can be substituted with each other.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy.

Further, the single bond dissociation energy of carbon and deuterium is higher than the single bond dissociation energy of carbon and hydrogen. Accordingly, the deuterium-substituted structure has an effect of increasing the thermal stability of the molecule and improving the service life of the device using the increased thermal stability.

When a compound is deposited on a silicon wafer, a material including deuterium tends to be packed so that the intermolecular distance is reduced. Further, when the surface of a thin film is observed using an atomic force microscope (AFM), it can be confirmed that the thin film made of a compound including deuterium is deposited with a more uniform surface without any aggregated portion.

The heterocyclic compound of Chemical Formula 1 of the present application has a deuterium substitution rate of more than 0% and 100% or less. The deuterium-substituted compound is characterized in that the energy in the ground state is further lower than that of the hydrogen-substituted compound, and the shorter the bond length between carbon and deuterium is, the smaller the molecular hardcore volume is. Accordingly, the electrical polarizability may be reduced and the intermolecular interaction can be weakened, so that when a device is manufactured, the device has a stabler stacking structure.

These characteristics induce an effect of lowering the crystallinity by creating the amorphous state of a thin film. That is, the heterocyclic compound represented by Chemical Formula 1 may be effective in improving the heat resistance of an OLED device, thereby improving the service life and driving characteristics.

In an exemplary embodiment of the present application, provided is a heterocyclic compound represented by the following Chemical Formula 1.

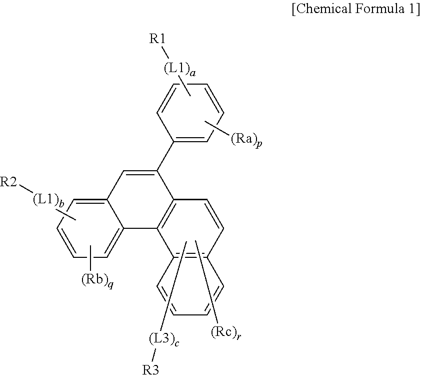

In Chemical Formula 1,
L1 to L3 are the same as or different from each other, and are each independently a direct bond; a substituted or unsubstituted C6 to C60 arylene group; or a substituted or unsubstituted C2 to C60 heteroarylene group,
R1 to R3 are the same as or different from each other, and are each independently hydrogen; deuterium; a substituted or unsubstituted amine group; a substituted or unsubstituted C6 to C60 aryl group; a substituted or unsubstituted C2 to C60 heteroaryl group; or —P(═O)RR′,
at least one of R1 to R3 is a substituted or unsubstituted amine group, and another one of R1 to R3 is a substituted or unsubstituted C2 to C60 heteroaryl group; or —P(═O)RR′,
R and R′ are the same as or different from each other, and are each independently hydrogen; deuterium; —CN; a substituted or unsubstituted C6 to C60 aryl group; or a substituted or unsubstituted C2 to C60 heteroaryl group,
Ra, Rb and Rc are the same as or different from each other, and are each independently hydrogen; deuterium; a halogen group; —CN; a substituted or unsubstituted C1 to C60 alkyl group; a substituted or unsubstituted C6 to C60 aryl group; or a substituted or unsubstituted C2 to C60 heteroaryl group,
a, b and c are an integer from 0 to 4, and when a, b and c are 2 or higher, substituents in the parenthesis are the same as or different from each other,
p is an integer from 0 to 4, and when p is 2 or higher, substituents in the parenthesis are the same as or different from each other,
q is an integer from 0 to 3, and when q is 2 or higher, substituents in the parenthesis are the same as or different from each other, and
r is an integer from 0 to 5, and when r is 2 or higher, substituents in the parenthesis are the same as or different from each other.

In an exemplary embodiment of the present application, Chemical Formula 1 may be represented by any one of the following Chemical Formulae 1-1 to 1-6.

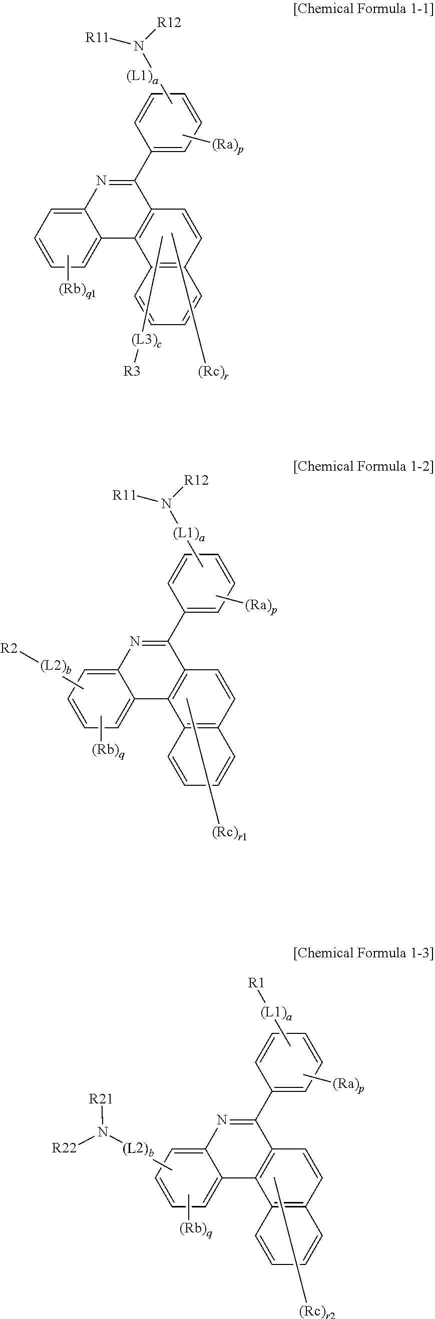

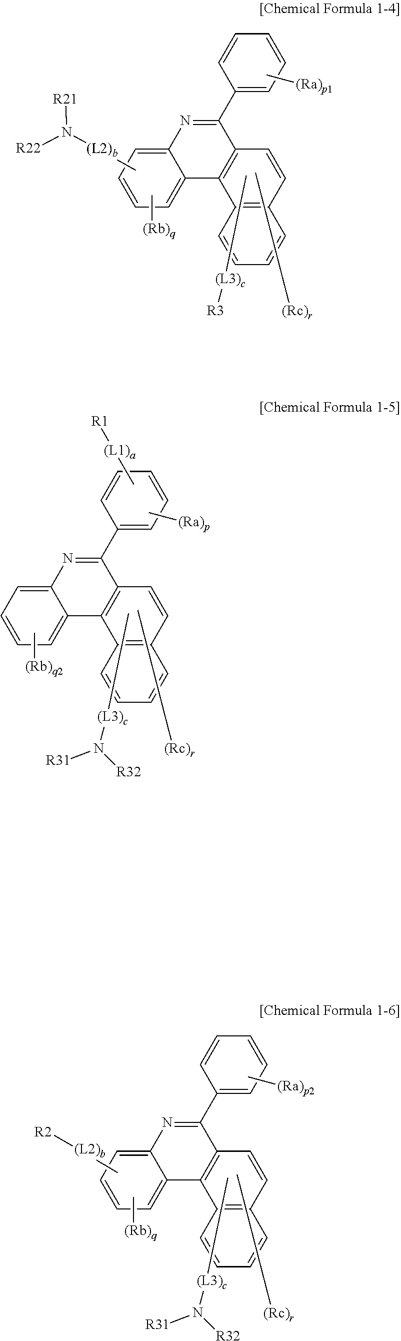

In Chemical Formulae 1-1 to 1-6,
L1 to L3, R1 to R3, Ra, Rb, Rc, a, b, c, p, q and r are the same as the definitions in Chemical Formula 1,
R11, R12, R21, R22, R31 and R32 are the same as or different from each other, and are each independently a substituted or unsubstituted C6 to C60 aryl group; or a substituted or unsubstituted C2 to C60 heteroaryl group,
p1 and p2 are each an integer from 0 to 5, and when p1 and p2 are 2 or higher, substituents in the parenthesis are the same as or different from each other,
q1 and q2 are each an integer from 0 to 4, and when q1 and q2 are 2 or higher, substituents in the parenthesis are the same as or different from each other, and
r1 and r2 are each an integer from 0 to 6, and when r1 and r2 are 2 or higher, substituents in the parenthesis are the same as or different from each other.

In an exemplary embodiment of the present application, Chemical Formula 1 may be represented by any one of the following Chemical Formulae 1-7 to 1-18.

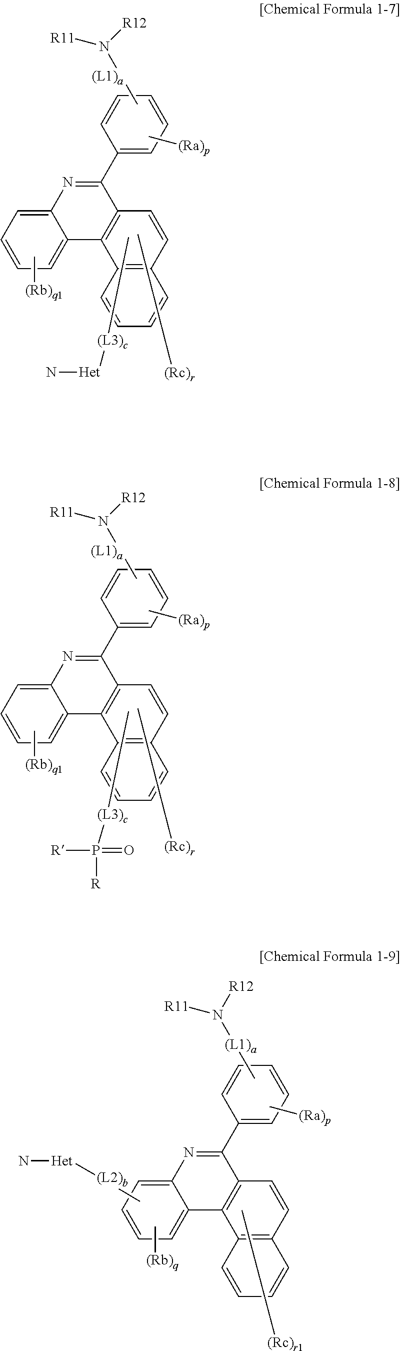

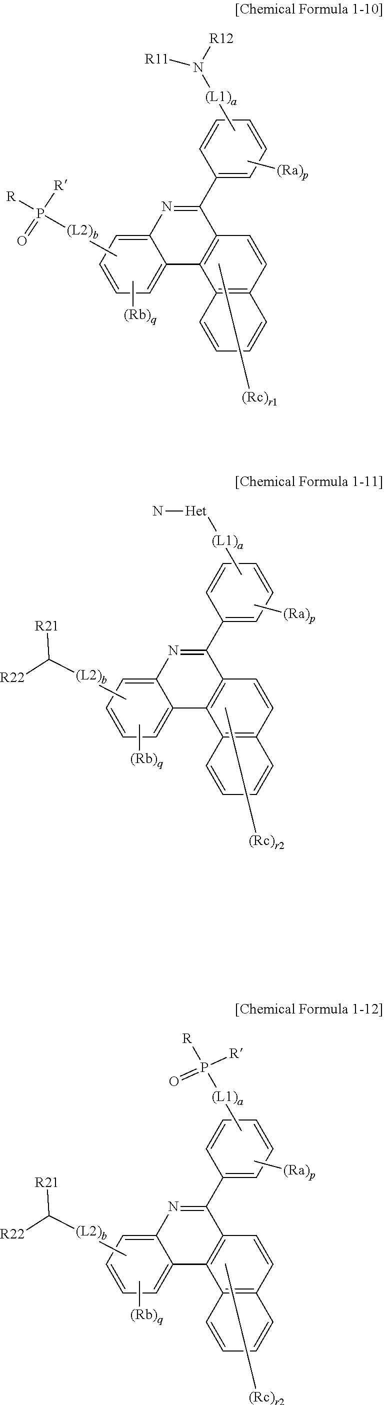

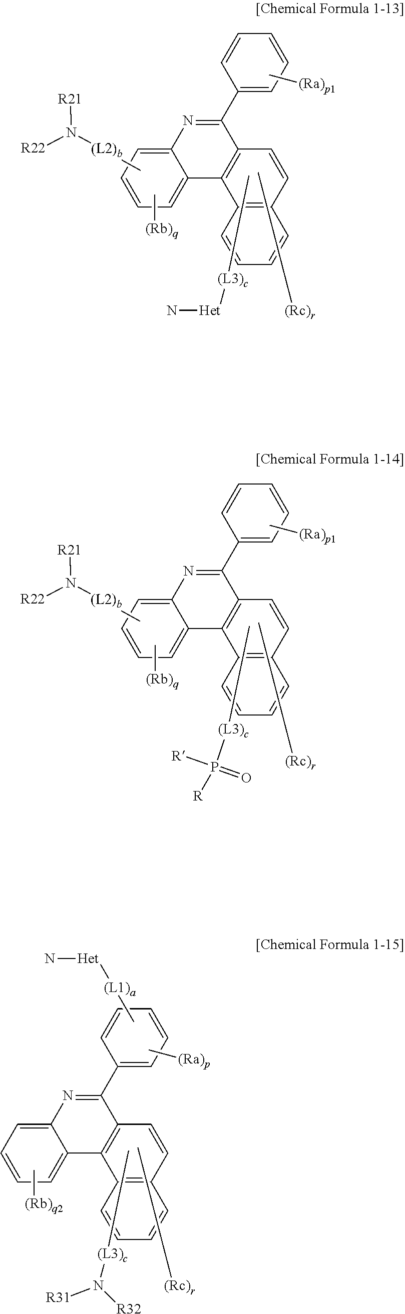

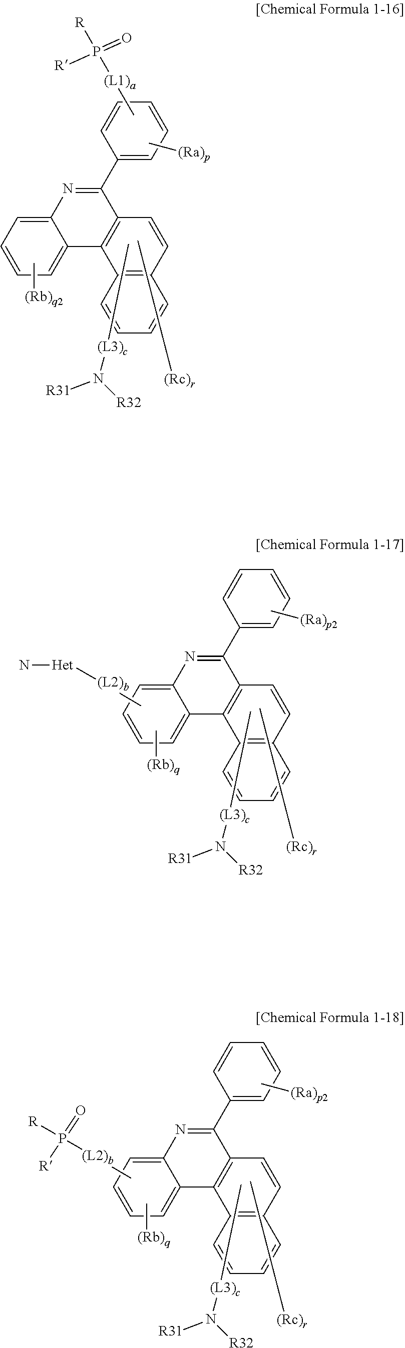

In Chemical Formulae 1-7 to 1-18,
L1 to L3, Ra, Rb, Rc, R, R′, a, b, c, p, q and r are the same as the definitions in Chemical Formula 1,
R11, R12, R21, R22, R31 and R32 are the same as or different from each other, and are each independently a substituted or unsubstituted C6 to C60 aryl group; or a substituted or unsubstituted C2 to C60 heteroaryl group,
N-Het is a C2 to C60 heteroaryl group which is substituted or unsubstituted and includes N,
p1 and p2 are each an integer from 0 to 5, and when p1 and p2 are 2 or higher, substituents in the parenthesis are the same as or different from each other,
q1 and q2 are each an integer from 0 to 4, and when q1 and q2 are 2 or higher, substituents in the parenthesis are the same as or different from each other, and
r1 and r2 are each an integer from 0 to 6, and when r1 and r2 are 2 or higher, substituents in the parenthesis are the same as or different from each other.

In an exemplary embodiment of the present application, R11, R12, R21, R22, R31 and R32 are the same as or different from each other, and may be each independently a substituted or unsubstituted C6 to C40 aryl group; or a substituted or unsubstituted C2 to C40 heteroaryl group.

In another exemplary embodiment, R11, R12, R21, R22, R31 and R32 are the same as or different from each other, and may be each independently a substituted or unsubstituted C6 to C20 aryl group; or a substituted or unsubstituted C2 to C20 heteroaryl group.

In still another exemplary embodiment, R11, R12, R21, R22, R31 and R32 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted phenanthrene group; a substituted or unsubstituted triphenylenyl group; a substituted or unsubstituted dibenzofuran group; or a substituted or unsubstituted dibenzothiophene group.

In yet another exemplary embodiment, R11, R12, R21, R22, R31 and R32 are the same as or different from each other, and may be each independently a substituted or unsubstituted phenyl group; a substituted or unsubstituted biphenyl group; a substituted or unsubstituted naphthyl group; a substituted or unsubstituted dibenzofuran group; or a substituted or unsubstituted dibenzothiophene group.

In still yet another exemplary embodiment, R11, R12, R21, R22, R31 and R32 are the same as or different from each other, and may be each independently a phenyl group which is unsubstituted or substituted with deuterium; a biphenyl group which is unsubstituted or substituted with deuterium; a naphthyl group which is unsubstituted or substituted with deuterium; a dibenzofuran group which is unsubstituted or substituted with deuterium; or a dibenzothiophene group which is unsubstituted or substituted with deuterium.

In an exemplary embodiment of the present application, the N-Het may be a C2 to C40 heteroaryl group which is substituted or unsubstituted and includes N.

In another exemplary embodiment, the N-Het may be a C2 to C20 heteroaryl group which is substituted or unsubstituted and includes N.

In still another exemplary embodiment, the N-Het may be a substituted or unsubstituted pyridine group; a substituted or unsubstituted pyrimidine group; a substituted or unsubstituted triazine group; a substituted or unsubstituted quinoline group; a substituted or unsubstituted benzimidazole group; or a substituted or unsubstituted phenanthroline group.

In yet another exemplary embodiment, the N-Het may be a pyridine group which is substituted with a phenyl group or a biphenyl group; a pyrimidine group which is substituted with a phenyl group or a biphenyl group; a triazine group which is substituted with a phenyl group or a biphenyl group; a quinoline group which is substituted with a phenyl group or a biphenyl group; a benzimidazole group which is substituted with a phenyl group or a biphenyl group; or a phenanthroline group which is unsubstituted or substituted with a phenyl group or a biphenyl group.

In still yet another exemplary embodiment, the N-Het may be a pyridine group which is substituted with a phenyl group; a pyrimidine group which is substituted with a phenyl group or a biphenyl group; a triazine group which is substituted with a phenyl group; a quinoline group which is substituted with a biphenyl group; a benzimidazole group which is substituted with a phenyl group; or a phenanthroline group which is unsubstituted or substituted with a phenyl group.

In an exemplary embodiment of the present application, L1 to L3 are the same as or different from each other, and may be each independently a direct bond; a substituted or unsubstituted C6 to C40 arylene group; or a substituted or unsubstituted C2 to C40 heteroarylene group.

In another exemplary embodiment, L1 to L3 are the same as or different from each other, and may be each independently a direct bond; a substituted or unsubstituted C6 to C20 arylene group; or a substituted or unsubstituted C2 to C20 heteroarylene group.

In still another exemplary embodiment, L1 to L3 are the same as or different from each other, and may be each independently a direct bond; or a substituted or unsubstituted C6 to C20 arylene group.

In yet another exemplary embodiment, L1 to L3 are the same as or different from each other, and may be each independently a direct bond; a substituted or unsubstituted phenylene group; a substituted or unsubstituted naphthylene group; or a substituted or unsubstituted biphenylene group.

In still yet another exemplary embodiment, L1 to L3 are the same as or different from each other, and may be each independently a direct bond; a phenylene group; a naphthylene group; or a biphenylene group.

In a further exemplary embodiment, L1 to L3 are the same as or different from each other, and may be each independently a direct bond; or a phenylene group.

In an exemplary embodiment of the present application, R1 to R3 are the same as or different from each other, and may be each independently hydrogen; deuterium; a substituted or unsubstituted amine group; a substituted or unsubstituted C6 to C40 aryl group; a substituted or unsubstituted C2 to C40 heteroaryl group; or —P(═O)RR′, any one of R1 to R3 is a substituted or unsubstituted amine group, and another one of R1 to R3 may be a substituted or unsubstituted C2 to C40 heteroaryl group; or —P(═O)RR′.

In another exemplary embodiment, R1 to R3 are the same as or different from each other, and may be each independently hydrogen; deuterium; an amine group which is unsubstituted or substituted with a substituted or unsubstituted C6 to C40 aryl group or a substituted or unsubstituted C2 to C40 heteroaryl group; a substituted or unsubstituted C6 to C40 aryl group; a substituted or unsubstituted C2 to C40 heteroaryl group; or —P(═O)RR′, any one of R1 to R3 is an amine group which is unsubstituted or substituted with a substituted or unsubstituted C6 to C40 aryl group or a substituted or unsubstituted C2 to C40 heteroaryl group, and another one of R1 to R3 may be a substituted or unsubstituted C2 to C40 heteroaryl group; or —P(═O)RR′.

In still another exemplary embodiment, R1 to R3 are the same as or different from each other, and may be each independently hydrogen; deuterium; an amine group which is unsubstituted or substituted with a substituted or unsubstituted C6 to C40 aryl group or a substituted or unsubstituted C2 to C40 heteroaryl group; a substituted or unsubstituted C2 to C40 heteroaryl group; or —P(═O)RR′, any one of R1 to R3 is an amine group which is unsubstituted or substituted with a substituted or unsubstituted C6 to C40 aryl group or a substituted or unsubstituted C2 to C40 heteroaryl group, and another one of R1 to R3 may be a substituted or unsubstituted C2 to C40 heteroaryl group; or —P(═O)RR′.

In yet another exemplary embodiment of the present application, R1 to R3 are the same as or different from each other, and may be each independently hydrogen; deuterium; an amine group which is unsubstituted or substituted with a substituted or unsubstituted C6 to C20 aryl group or a substituted or unsubstituted C2 to C20 heteroaryl group; a substituted or unsubstituted C2 to C20 heteroaryl group; or —P(═O)RR′, any one of R1 to R3 is an amine group which is unsubstituted or substituted with a substituted or unsubstituted C6 to C20 aryl group or a substituted or unsubstituted C2 to C20 heteroaryl group, and another one of R1 to R3 may be a substituted or unsubstituted C2 to C20 heteroaryl group; or —P(═O)RR′.

In still yet another exemplary embodiment, R1 to R3 are the same as or different from each other, and are each independently hydrogen; deuterium; an amine group which is unsubstituted or substituted with a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted phenanthrene group, a substituted or unsubstituted triphenylenyl group, a substituted or unsubstituted dibenzofuran group or a substituted or unsubstituted dibenzothiophene group; a substituted or unsubstituted pyridine group; a substituted or unsubstituted pyrimidine group; a substituted or unsubstituted triazine group; a substituted or unsubstituted quinoline group; a substituted or unsubstituted benzimidazole group; a substituted or unsubstituted phenanthroline group; or —P(═O)RR′, any one of R1 to R3 is an amine group which is unsubstituted or substituted with a substituted or unsubstituted phenyl group, a substituted or unsubstituted biphenyl group, a substituted or unsubstituted terphenyl group, a substituted or unsubstituted naphthyl group, a substituted or unsubstituted dibenzofuran group or a substituted or unsubstituted dibenzothiophene group, and another one of R1 to R3 may be a substituted or unsubstituted pyridine group; a substituted or unsubstituted pyrimidine group; a substituted or unsubstituted triazine group; a substituted or unsubstituted quinoline group; a substituted or unsubstituted benzimidazole group; a substituted or unsubstituted phenanthroline group; or —P(═O)RR′.

In a further exemplary embodiment, R1 to R3 are the same as or different from each other, and are each independently hydrogen; deuterium; an amine group which is substituted with a phenyl group unsubstituted or substituted with deuterium, a biphenyl group unsubstituted or substituted with deuterium, a naphthyl group unsubstituted or substituted with deuterium, a dibenzofuran group unsubstituted or substituted with deuterium, or a dibenzothiophene group unsubstituted or substituted with deuterium; a pyridine group which is substituted with deuterium, or a phenyl group unsubstituted or substituted with deuterium; a pyrimidine group which is substituted with deuterium, a phenyl group unsubstituted or substituted with deuterium or a biphenyl group; a triazine group which is substituted with a phenyl group unsubstituted or substituted with deuterium; a quinoline group which is substituted with deuterium, a phenyl group or a biphenyl group; a benzimidazole group which is substituted with deuterium or a phenyl group unsubstituted or substituted with deuterium; a phenanthroline group which is unsubstituted or substituted with deuterium or a phenyl group unsubstituted or substituted with deuterium; or —P(═O)RR′, any one of R1 to R3 is an amine group which is substituted with a phenyl group unsubstituted or substituted with deuterium, a biphenyl group unsubstituted or substituted with deuterium, a naphthyl group unsubstituted or substituted with deuterium, a dibenzofuran group unsubstituted or substituted with deuterium, or a dibenzothiophene group unsubstituted or substituted with deuterium, and another one of R1 to R3 may be a pyridine group which is substituted with deuterium or a phenyl group unsubstituted or substituted with deuterium; a pyrimidine group which is substituted with deuterium, a phenyl group unsubstituted or substituted with deuterium or a biphenyl group; a triazine group which is substituted with a phenyl group unsubstituted or substituted with deuterium; a quinoline group which is substituted with deuterium, a phenyl group or a biphenyl group; a benzimidazole group which is substituted with deuterium or a phenyl group unsubstituted or substituted with deuterium; a phenanthroline group which is unsubstituted or substituted with deuterium or a phenyl group unsubstituted or substituted with deuterium; or —P(═O)RR′.

In an exemplary embodiment of the present application, R and R′ are the same as or different from each other, and may be each independently a substituted or unsubstituted C6 to C60 aryl group.

In another exemplary embodiment, R and R′ are the same as or different from each other, and may be each independently a substituted or unsubstituted C6 to C40 aryl group.

In still another exemplary embodiment, R and R′ are the same as or different from each other, and may be each independently a substituted or unsubstituted C6 to C20 aryl group.

In yet another exemplary embodiment, R and R′ are the same as or different from each other, and may be each independently a substituted or unsubstituted phenyl group; a substituted or unsubstituted biphenyl group; a substituted or unsubstituted terphenyl group; or a substituted or unsubstituted naphthyl group.

In still yet another exemplary embodiment, R and R′ are the same as or different from each other, and may be each independently a phenyl group which is unsubstituted or substituted with deuterium; or a biphenyl group which is unsubstituted or substituted with deuterium.

In a further exemplary embodiment, R and R′ may be a phenyl group which is unsubstituted or substituted with deuterium.

In an exemplary embodiment of the present application, Ra, Rb and Rc are the same as or different from each other, and may be each independently hydrogen; deuterium; a substituted or unsubstituted C1 to C40 alkyl group; a substituted or unsubstituted C6 to C40 aryl group; or a substituted or unsubstituted C2 to C40 heteroaryl group.

In another exemplary embodiment, Ra, Rb and Rc are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In still another exemplary embodiment, Ra, Rb and Rc are the same as or different from each other, and may be each independently hydrogen; deuterium or a substituted or unsubstituted C6 to C20 aryl group.

In yet another exemplary embodiment, Ra, Rb and Rc are the same as or different from each other, and may be each independently hydrogen; or deuterium.

In an exemplary embodiment of the present application, a, b and c may be an integer from 0 to 3, and when a, b and c are 2 or higher, substituents in the parenthesis may be the same as or different from each other.

In another exemplary embodiment, a, b and c may be an integer from 0 to 2, and when a, b and c are 2 or higher, substituents in the parenthesis may be the same as or different from each other.

In still another exemplary embodiment, a, b and c may be 0 or 1.

In yet another exemplary embodiment, a, b and c may be 1.

In still yet another exemplary embodiment, a, b and c may be 0.

In an exemplary embodiment of the present application, p may be an integer from 0 to 4, and when p is 2 or higher, substituents in the parenthesis may be the same as or different from each other.

In another exemplary embodiment, p may be an integer from 0 to 3, and when p is 2 or higher, substituents in the parenthesis may be the same as or different from each other.

In still another exemplary embodiment, p may be an integer from 0 to 2, and when p is 2 or higher, substituents in the parenthesis may be the same as or different from each other.

In yet another exemplary embodiment, p may be 0 or 1.

In still yet another exemplary embodiment, p may be 1.

In a further exemplary embodiment, p may be 0.

In an exemplary embodiment of the present application, q may be an integer from 0 to 3, and when q is 2 or higher, substituents in the parenthesis may be the same as or different from each other.

In another exemplary embodiment, q may be an integer from 0 to 2, and when q is 2 or higher, substituents in the parenthesis may be the same as or different from each other.

In still another exemplary embodiment, q may be 0 or 1.

In yet another exemplary embodiment, q may be 1.

In still yet another exemplary embodiment, q may be 0.

In an exemplary embodiment of the present application, r may be an integer from 0 to 5, and when r is 2 or higher, substituents in the parenthesis may be the same as or different from each other.

In another exemplary embodiment, r may be an integer from 0 to 4, and when r is 2 or higher, substituents in the parenthesis may be the same as or different from each other.

In still another exemplary embodiment, r may be an integer from 0 to 3, and when r is 2 or higher, substituents in the parenthesis may be the same as or different from each other.

In yet another exemplary embodiment, r may be an integer from 0 to 2, and when r is 2 or higher, substituents in the parenthesis may be the same as or different from each other.

In still yet another exemplary embodiment, r may be 0 or 1.

In a further exemplary embodiment, r may be 1.

In another further exemplary embodiment, r may be 0.

In an exemplary embodiment of the present application, the deuterium content of the heterocyclic compound of Chemical Formula 1 may be 0%, or 30% to 100%.

In an exemplary embodiment of the present application, the deuterium content of the heterocyclic compound of Chemical Formula 1 may be 0%, or 50% to 100%.

In an exemplary embodiment of the present application, the deuterium content of the heterocyclic compound of Chemical Formula 1 may be 0%, or 70% to 100%.

In an exemplary embodiment of the present application, the deuterium content of the heterocyclic compound of Chemical Formula 1 may be 0%, or 90% to 100%.

In an exemplary embodiment of the present application, the deuterium content of the heterocyclic compound of Chemical Formula 1 may be 0%.

In an exemplary embodiment of the present application, the deuterium content of the heterocyclic compound of Chemical Formula 1 may be 30% to 100%.

In an exemplary embodiment of the present application, the deuterium content of the heterocyclic compound of Chemical Formula 1 may be 50% to 100%.

In an exemplary embodiment of the present application, the deuterium content of the heterocyclic compound of Chemical Formula 1 may be 70% to 100%.

In an exemplary embodiment of the present application, the deuterium content of the heterocyclic compound of Chemical Formula 1 may be 90% to 100%.

In an exemplary embodiment of the present application, provided is a heterocyclic compound in which Chemical Formula 1 is represented by any one of the following compounds.

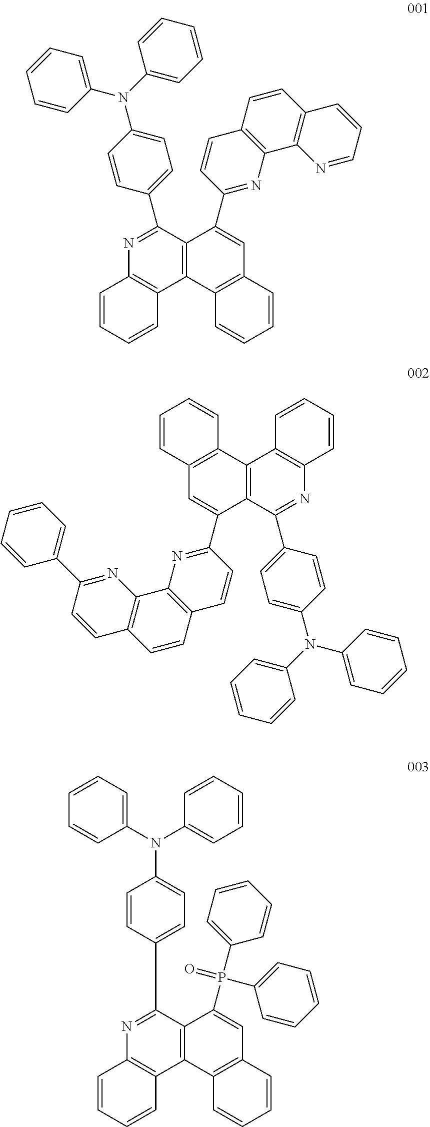 [SMILES: c1ccc(N(c2ccccc2)c2ccc(-c3nc4ccccc4c4c3c(-c3ccc5ccc6cccnc6c5n3)cc3ccccc34)cc2)cc1 ; c1ccc(-c2ccc3ccc4ccc(-c5cc6ccccc6c6c5c(-c5ccc(N(c7ccccc7)c7ccccc7)cc5)nc5ccccc56)nc4c3n2)cc1 ; O=P(c1ccccc1)(c1ccccc1)c1cc2ccccc2c2c1c(-c1ccc(N(c3ccccc3)c3ccccc3)cc1)nc1ccccc12]

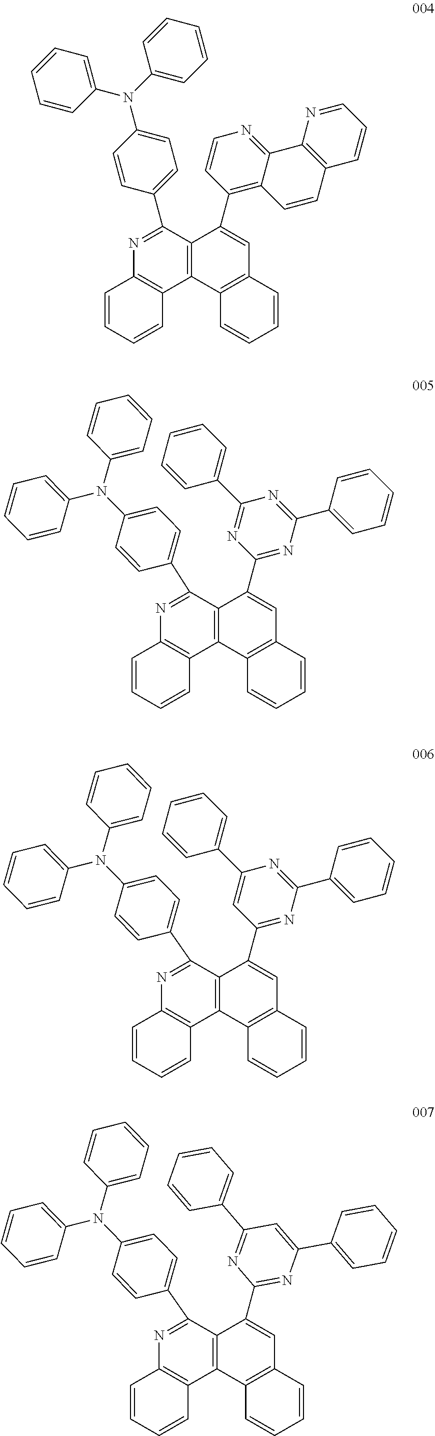 [SMILES: c1ccc(N(c2ccccc2)c2ccc(-c3nc4ccccc4c4c3c(-c3ccnc5c3ccc3cccnc35)cc3ccccc34)cc2)cc1 ; c1ccc(-c2nc(-c3ccccc3)nc(-c3cc4ccccc4c4c3c(-c3ccc(N(c5ccccc5)c5ccccc5)cc3)nc3ccccc34)n2)cc1 ; c1ccc(-c2cc(-c3cc4ccccc4c4c3c(-c3ccc(N(c5ccccc5)c5ccccc5)cc3)nc3ccccc34)nc(-c3ccccc3)n2)cc1 ; c1ccc(-c2cc(-c3ccccc3)nc(-c3cc4ccccc4c4c3c(-c3ccc(N(c5ccccc5)c5ccccc5)cc3)nc3ccccc34)n2)cc1]

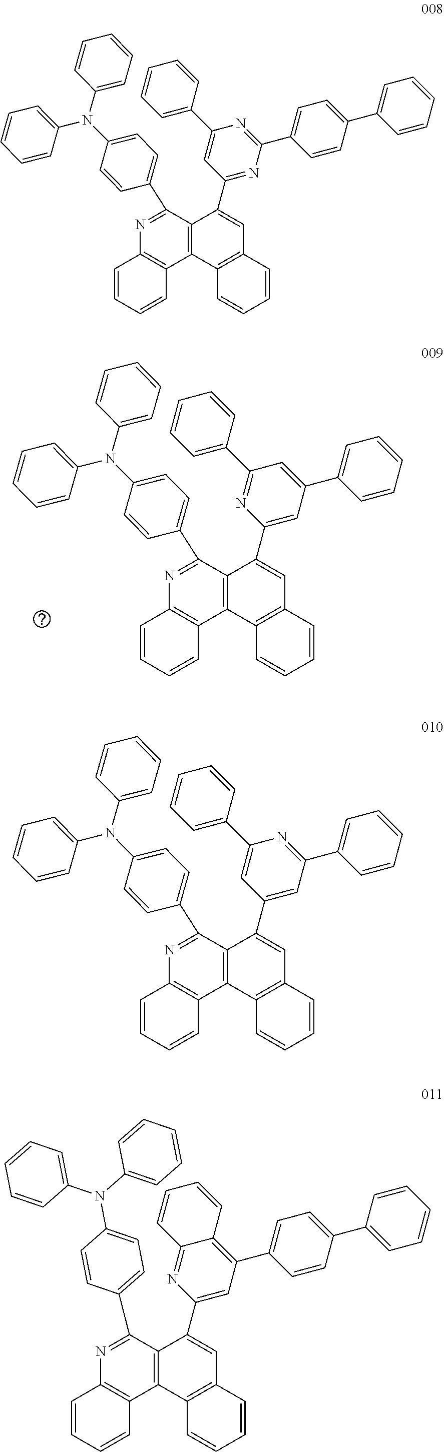 [SMILES: c1ccc(-c2ccc(-c3nc(-c4ccccc4)cc(-c4cc5ccccc5c5c4c(-c4ccc(N(c6ccccc6)c6ccccc6)cc4)nc4ccccc45)n3)cc2)cc1 ; c1ccc(-c2cc(-c3ccccc3)nc(-c3cc4ccccc4c4c3c(-c3ccc(N(c5ccccc5)c5ccccc5)cc3)nc3ccccc34)c2)cc1 ; c1ccc(-c2cc(-c3cc4ccccc4c4c3c(-c3ccc(N(c5ccccc5)c5ccccc5)cc3)nc3ccccc34)cc(-c3ccccc3)n2)cc1 ; c1ccc(-c2ccc(-c3cc(-c4cc5ccccc5c5c4c(-c4ccc(N(c6ccccc6)c6ccccc6)cc4)nc4ccccc45)nc4ccccc34)cc2)cc1]

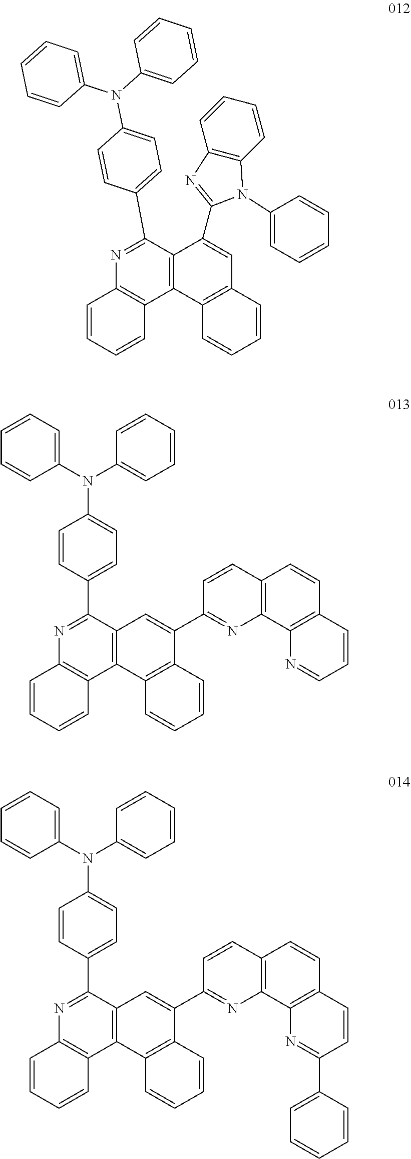 [SMILES: c1ccc(N(c2ccccc2)c2ccc(-c3nc4ccccc4c4c3c(-c3nc5ccccc5n3-c3ccccc3)cc3ccccc34)cc2)cc1 ; c1ccc(N(c2ccccc2)c2ccc(-c3nc4ccccc4c4c3cc(-c3ccc5ccc6cccnc6c5n3)c3ccccc34)cc2)cc1 ; c1ccc(-c2ccc3ccc4ccc(-c5cc6c(-c7ccc(N(c8ccccc8)c8ccccc8)cc7)nc7ccccc7c6c6ccccc56)nc4c3n2)cc1]

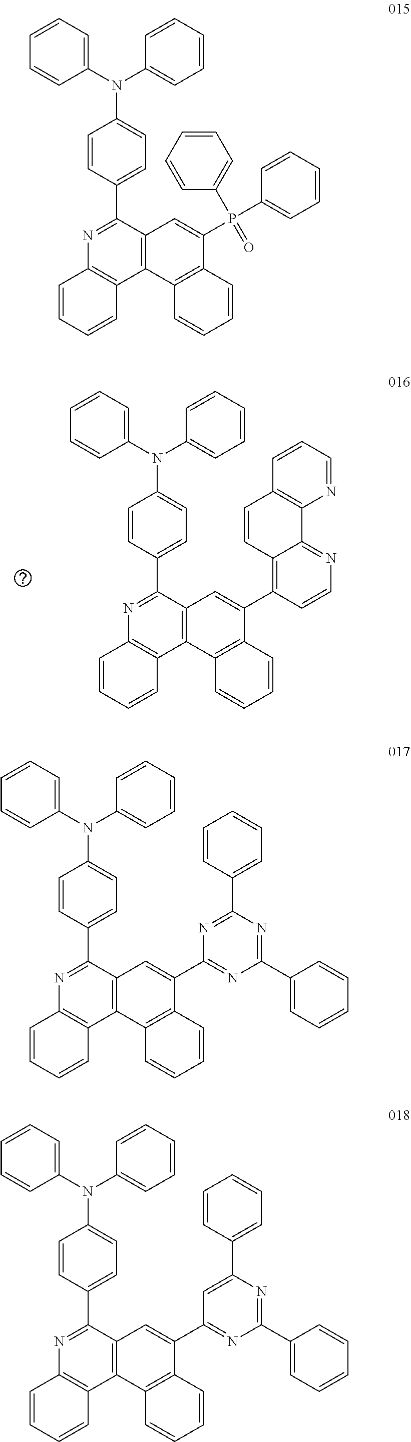 [SMILES: O=P(c1ccccc1)(c1ccccc1)c1cc2c(-c3ccc(N(c4ccccc4)c4ccccc4)cc3)nc3ccccc3c2c2ccccc12 ; c1ccc(N(c2ccccc2)c2ccc(-c3nc4ccccc4c4c3cc(-c3ccnc5c3ccc3cccnc35)c3ccccc34)cc2)cc1 ; c1ccc(-c2nc(-c3ccccc3)nc(-c3cc4c(-c5ccc(N(c6ccccc6)c6ccccc6)cc5)nc5ccccc5c4c4ccccc34)n2)cc1 ; c1ccc(-c2cc(-c3cc4c(-c5ccc(N(c6ccccc6)c6ccccc6)cc5)nc5ccccc5c4c4ccccc34)nc(-c3ccccc3)n2)cc1]

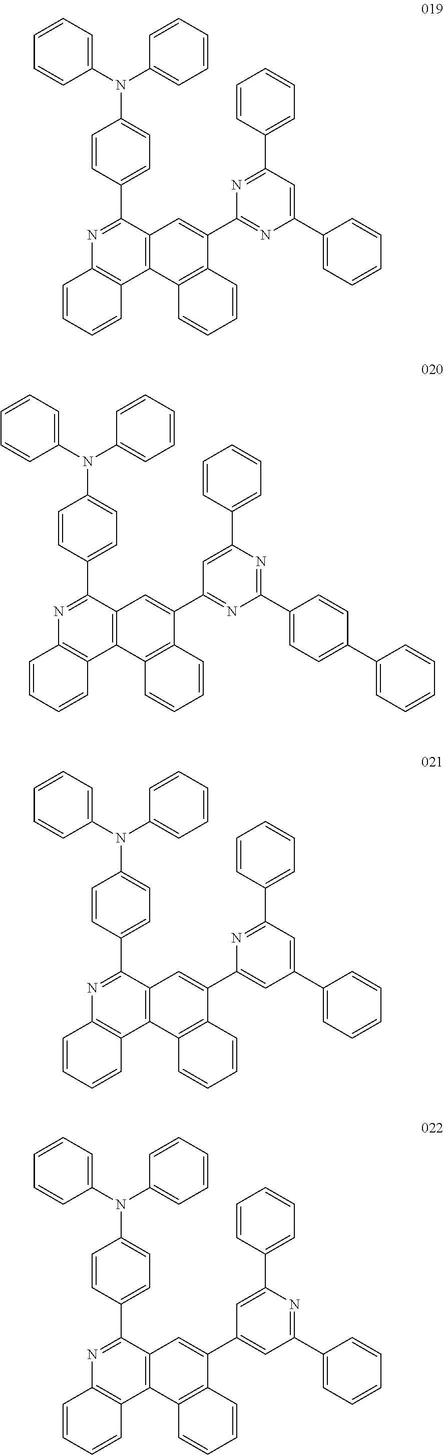 [SMILES: c1ccc(-c2cc(-c3ccccc3)nc(-c3cc4c(-c5ccc(N(c6ccccc6)c6ccccc6)cc5)nc5ccccc5c4c4ccccc34)n2)cc1 ; c1ccc(-c2ccc(-c3nc(-c4ccccc4)cc(-c4cc5c(-c6ccc(N(c7ccccc7)c7ccccc7)cc6)nc6ccccc6c5c5ccccc45)n3)cc2)cc1 ; c1ccc(-c2cc(-c3ccccc3)nc(-c3cc4c(-c5ccc(N(c6ccccc6)c6ccccc6)cc5)nc5ccccc5c4c4ccccc34)c2)cc1 ; c1ccc(-c2cc(-c3cc4c(-c5ccc(N(c6ccccc6)c6ccccc6)cc5)nc5ccccc5c4c4ccccc34)cc(-c3ccccc3)n2)cc1]

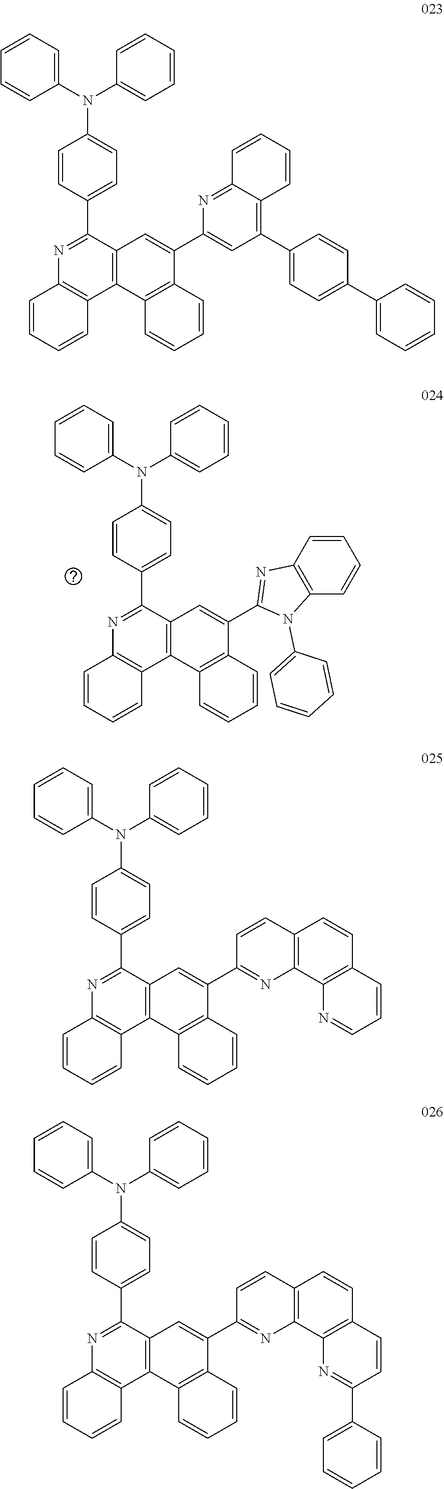 [SMILES: c1ccc(-c2ccc(-c3cc(-c4cc5c(-c6ccc(N(c7ccccc7)c7ccccc7)cc6)nc6ccccc6c5c5ccccc45)nc4ccccc34)cc2)cc1 ; c1ccc(N(c2ccccc2)c2ccc(-c3nc4ccccc4c4c3cc(-c3nc5ccccc5n3-c3ccccc3)c3ccccc34)cc2)cc1 ; c1ccc(N(c2ccccc2)c2ccc(-c3nc4ccccc4c4c3cc(-c3ccc5ccc6cccnc6c5n3)c3ccccc34)cc2)cc1 ; c1ccc(-c2ccc3ccc4ccc(-c5cc6c(-c7ccc(N(c8ccccc8)c8ccccc8)cc7)nc7ccccc7c6c6ccccc56)nc4c3n2)cc1]

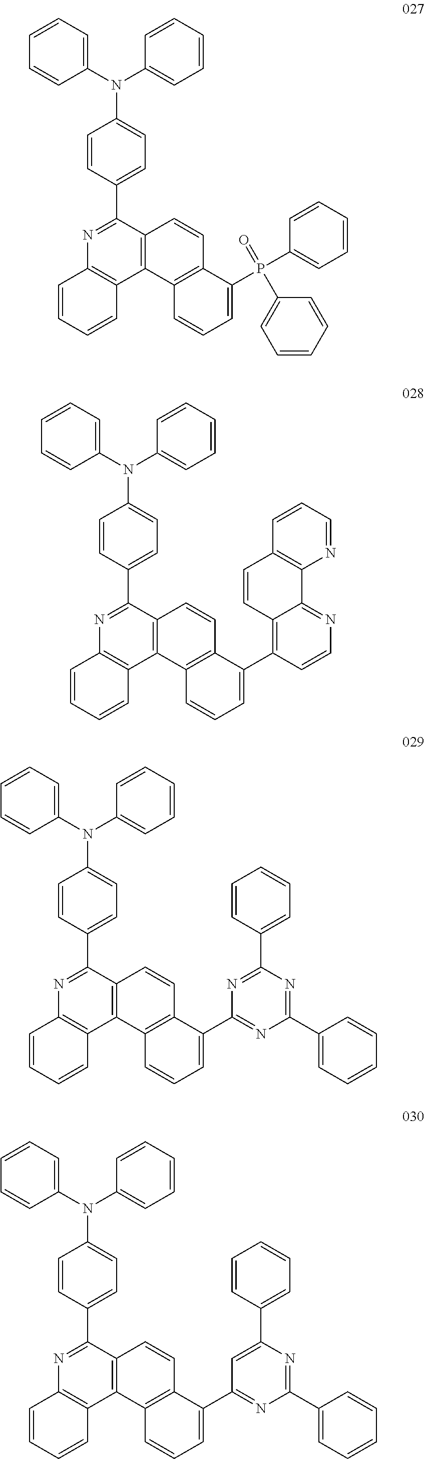 [SMILES: O=P(c1ccccc1)(c1ccccc1)c1cccc2c1ccc1c(-c3ccc(N(c4ccccc4)c4ccccc4)cc3)nc3ccccc3c12 ; c1ccc(N(c2ccccc2)c2ccc(-c3nc4ccccc4c4c3ccc3c(-c5ccnc6c5ccc5cccnc56)cccc34)cc2)cc1 ; c1ccc(-c2nc(-c3ccccc3)nc(-c3cccc4c3ccc3c(-c5ccc(N(c6ccccc6)c6ccccc6)cc5)nc5ccccc5c34)n2)cc1 ; c1ccc(-c2cc(-c3cccc4c3ccc3c(-c5ccc(N(c6ccccc6)c6ccccc6)cc5)nc5ccccc5c34)nc(-c3ccccc3)n2)cc1]

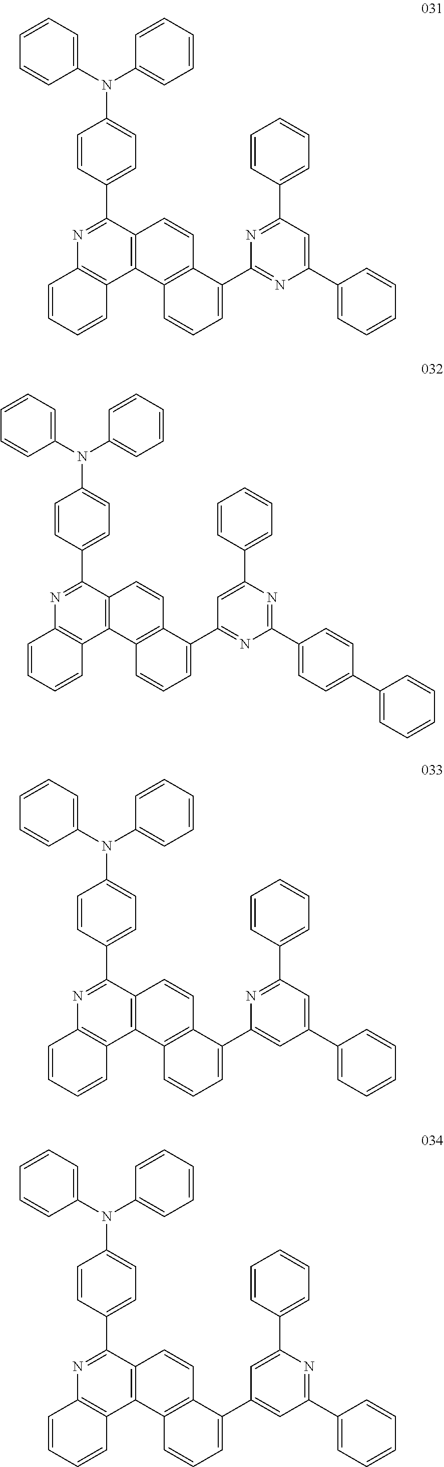 [SMILES: c1ccc(-c2cc(-c3ccccc3)nc(-c3cccc4c3ccc3c(-c5ccc(N(c6ccccc6)c6ccccc6)cc5)nc5ccccc5c34)n2)cc1 ; c1ccc(-c2ccc(-c3nc(-c4ccccc4)cc(-c4cccc5c4ccc4c(-c6ccc(N(c7ccccc7)c7ccccc7)cc6)nc6ccccc6c45)n3)cc2)cc1 ; c1ccc(-c2cc(-c3ccccc3)nc(-c3cccc4c3ccc3c(-c5ccc(N(c6ccccc6)c6ccccc6)cc5)nc5ccccc5c34)c2)cc1 ; c1ccc(-c2cc(-c3cccc4c3ccc3c(-c5ccc(N(c6ccccc6)c6ccccc6)cc5)nc5ccccc5c34)cc(-c3ccccc3)n2)cc1]

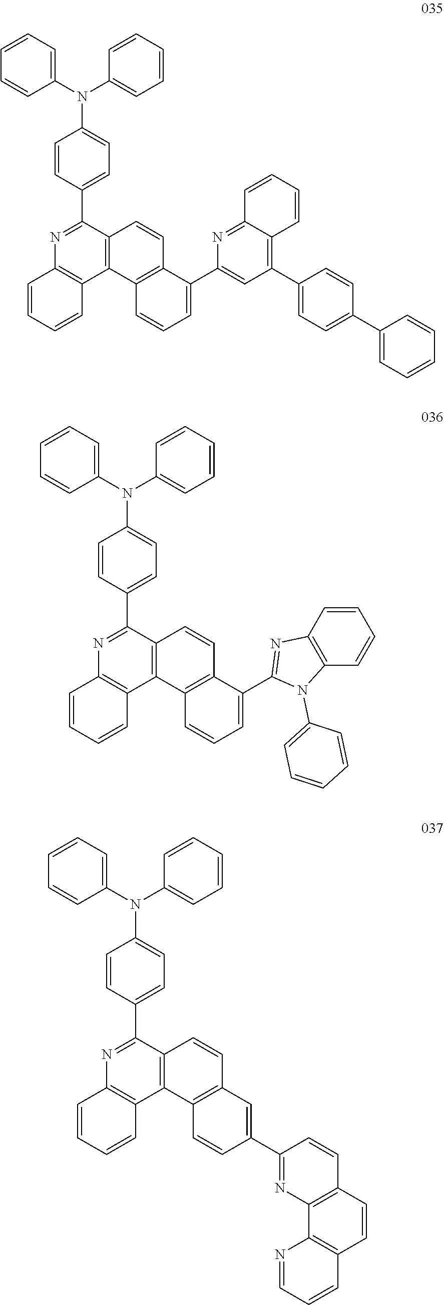 [SMILES: c1ccc(-c2ccc(-c3cc(-c4cccc5c4ccc4c(-c6ccc(N(c7ccccc7)c7ccccc7)cc6)nc6ccccc6c45)nc4ccccc34)cc2)cc1 ; c1ccc(N(c2ccccc2)c2ccc(-c3nc4ccccc4c4c3ccc3c(-c5nc6ccccc6n5-c5ccccc5)cccc34)cc2)cc1 ; c1ccc(N(c2ccccc2)c2ccc(-c3nc4ccccc4c4c3ccc3cc(-c5ccc6ccc7cccnc7c6n5)ccc34)cc2)cc1]

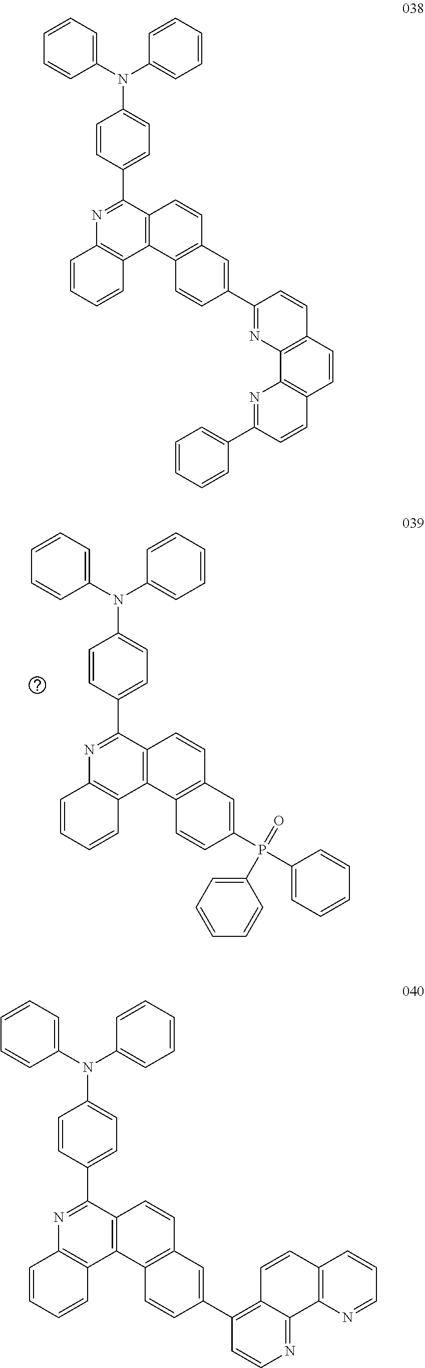 [SMILES: c1ccc(-c2ccc3ccc4ccc(-c5ccc6c(ccc7c(-c8ccc(N(c9ccccc9)c9ccccc9)cc8)nc8ccccc8c76)c5)nc4c3n2)cc1 ; O=P(c1ccccc1)(c1ccccc1)c1ccc2c(ccc3c(-c4ccc(N(c5ccccc5)c5ccccc5)cc4)nc4ccccc4c32)c1 ; c1ccc(N(c2ccccc2)c2ccc(-c3nc4ccccc4c4c3ccc3cc(-c5ccnc6c5ccc5cccnc56)ccc34)cc2)cc1]

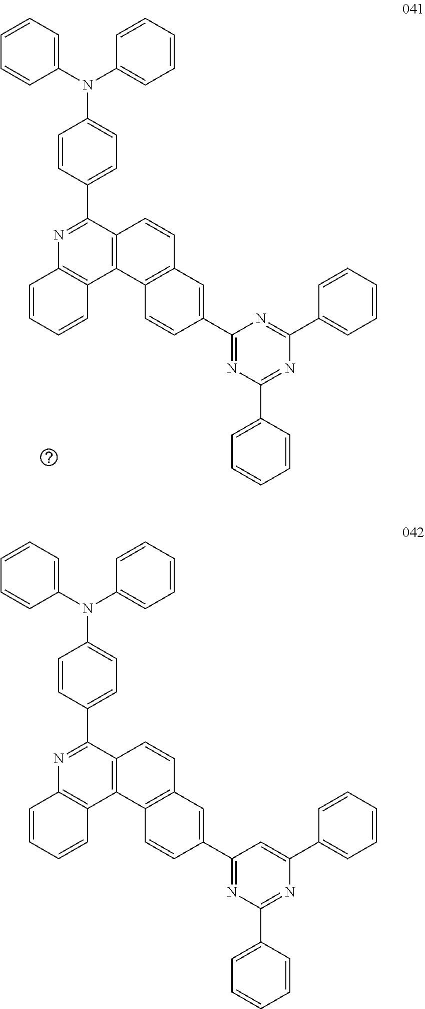 [SMILES: c1ccc(-c2nc(-c3ccccc3)nc(-c3ccc4c(ccc5c(-c6ccc(N(c7ccccc7)c7ccccc7)cc6)nc6ccccc6c54)c3)n2)cc1 ; c1ccc(-c2cc(-c3ccc4c(ccc5c(-c6ccc(N(c7ccccc7)c7ccccc7)cc6)nc6ccccc6c54)c3)nc(-c3ccccc3)n2)cc1]

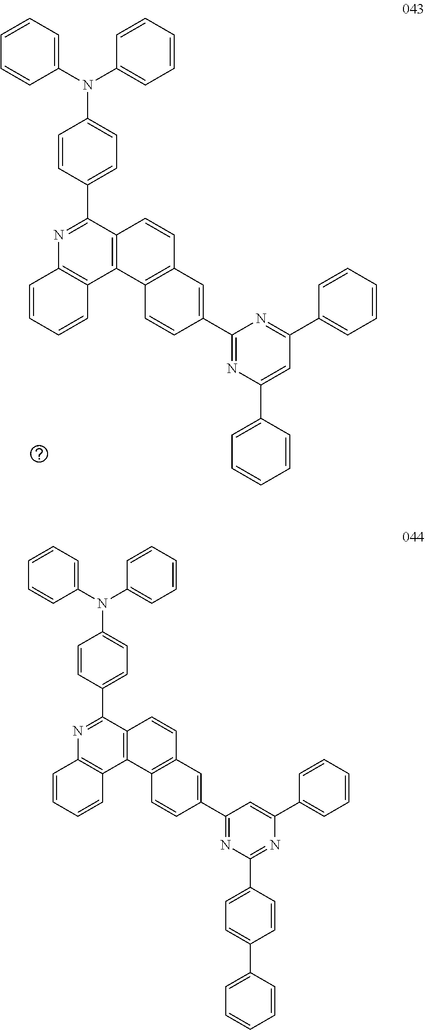 [SMILES: c1ccc(-c2cc(-c3ccccc3)nc(-c3ccc4c(ccc5c(-c6ccc(N(c7ccccc7)c7ccccc7)cc6)nc6ccccc6c54)c3)n2)cc1 ; c1ccc(-c2ccc(-c3nc(-c4ccccc4)cc(-c4ccc5c(ccc6c(-c7ccc(N(c8ccccc8)c8ccccc8)cc7)nc7ccccc7c65)c4)n3)cc2)cc1]

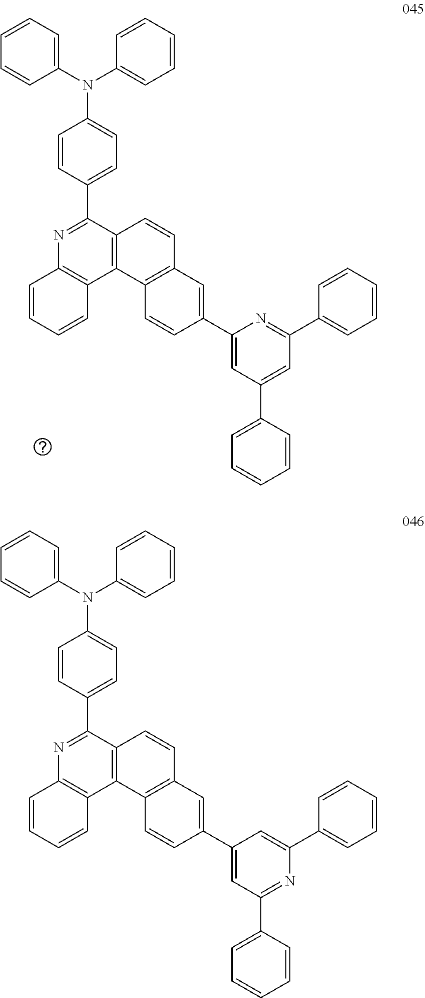 [SMILES: c1ccc(-c2cc(-c3ccccc3)nc(-c3ccc4c(ccc5c(-c6ccc(N(c7ccccc7)c7ccccc7)cc6)nc6ccccc6c54)c3)c2)cc1 ; c1ccc(-c2cc(-c3ccc4c(ccc5c(-c6ccc(N(c7ccccc7)c7ccccc7)cc6)nc6ccccc6c54)c3)cc(-c3ccccc3)n2)cc1]

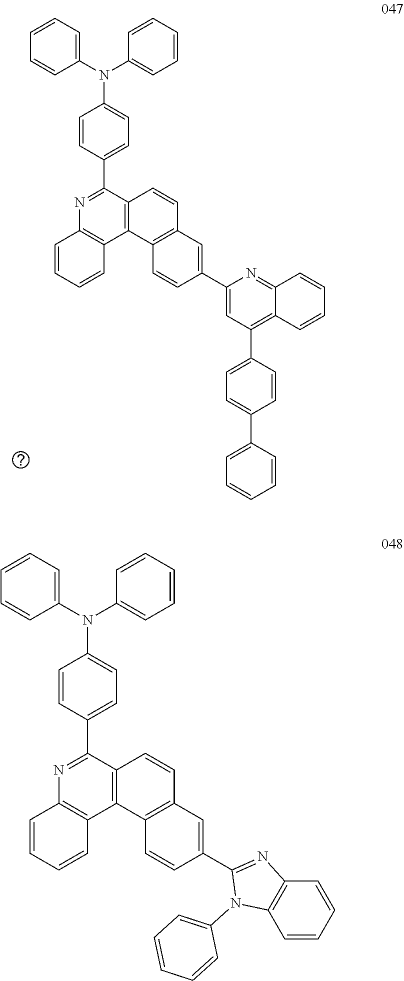 [SMILES: c1ccc(-c2ccc(-c3cc(-c4ccc5c(ccc6c(-c7ccc(N(c8ccccc8)c8ccccc8)cc7)nc7ccccc7c65)c4)nc4ccccc34)cc2)cc1 ; c1ccc(N(c2ccccc2)c2ccc(-c3nc4ccccc4c4c3ccc3cc(-c5nc6ccccc6n5-c5ccccc5)ccc34)cc2)cc1]

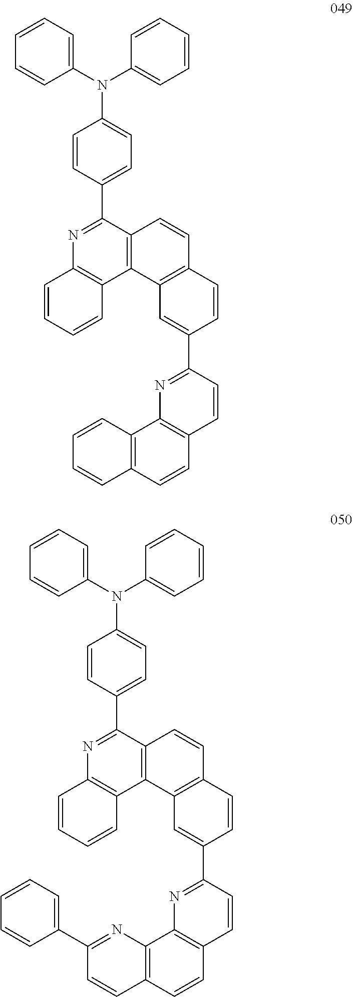

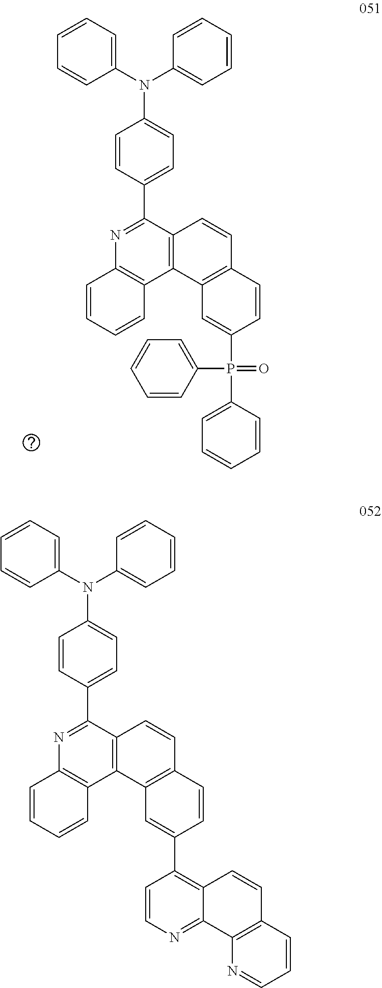 [SMILES: O=P(c1ccccc1)(c1ccccc1)c1ccc2ccc3c(-c4ccc(N(c5ccccc5)c5ccccc5)cc4)nc4ccccc4c3c2c1 ; c1ccc(N(c2ccccc2)c2ccc(-c3nc4ccccc4c4c3ccc3ccc(-c5ccnc6c5ccc5cccnc56)cc34)cc2)cc1]

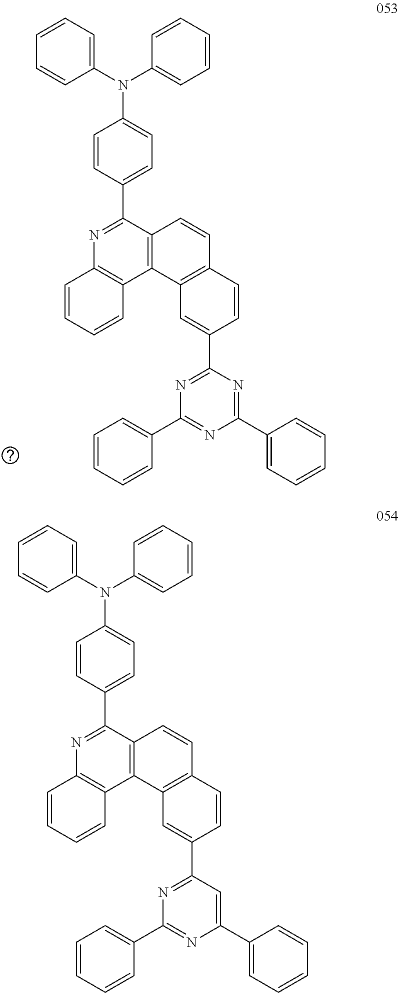 [SMILES: c1ccc(-c2nc(-c3ccccc3)nc(-c3ccc4ccc5c(-c6ccc(N(c7ccccc7)c7ccccc7)cc6)nc6ccccc6c5c4c3)n2)cc1 ; c1ccc(-c2cc(-c3ccc4ccc5c(-c6ccc(N(c7ccccc7)c7ccccc7)cc6)nc6ccccc6c5c4c3)nc(-c3ccccc3)n2)cc1]

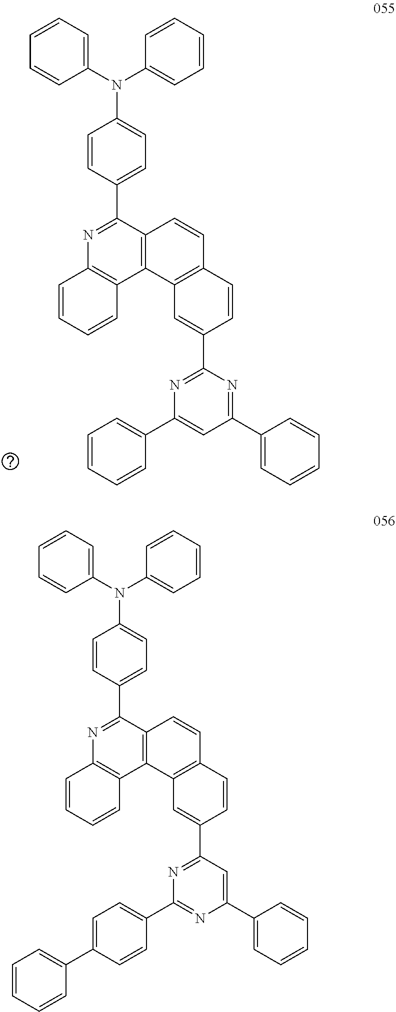 [SMILES: c1ccc(-c2cc(-c3ccccc3)nc(-c3ccc4ccc5c(-c6ccc(N(c7ccccc7)c7ccccc7)cc6)nc6ccccc6c5c4c3)n2)cc1 ; c1ccc(-c2ccc(-c3nc(-c4ccccc4)cc(-c4ccc5ccc6c(-c7ccc(N(c8ccccc8)c8ccccc8)cc7)nc7ccccc7c6c5c4)n3)cc2)cc1]

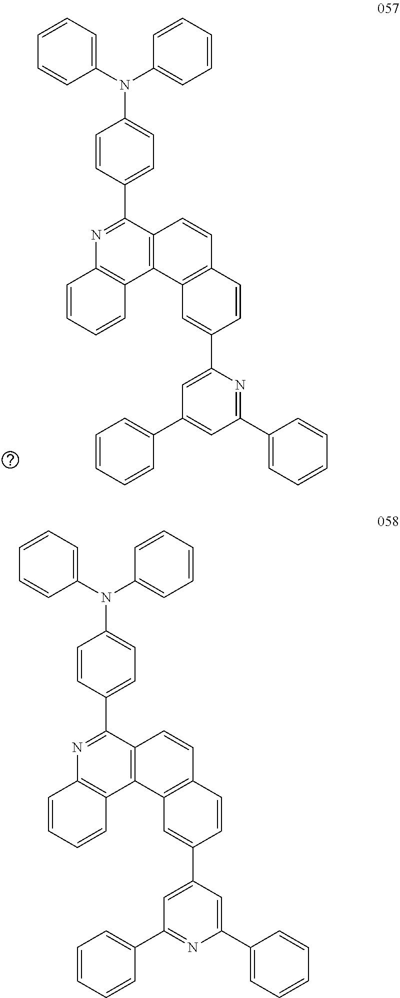 [SMILES: c1ccc(-c2cc(-c3ccccc3)nc(-c3ccc4ccc5c(-c6ccc(N(c7ccccc7)c7ccccc7)cc6)nc6ccccc6c5c4c3)c2)cc1 ; c1ccc(-c2cc(-c3ccc4ccc5c(-c6ccc(N(c7ccccc7)c7ccccc7)cc6)nc6ccccc6c5c4c3)cc(-c3ccccc3)n2)cc1]

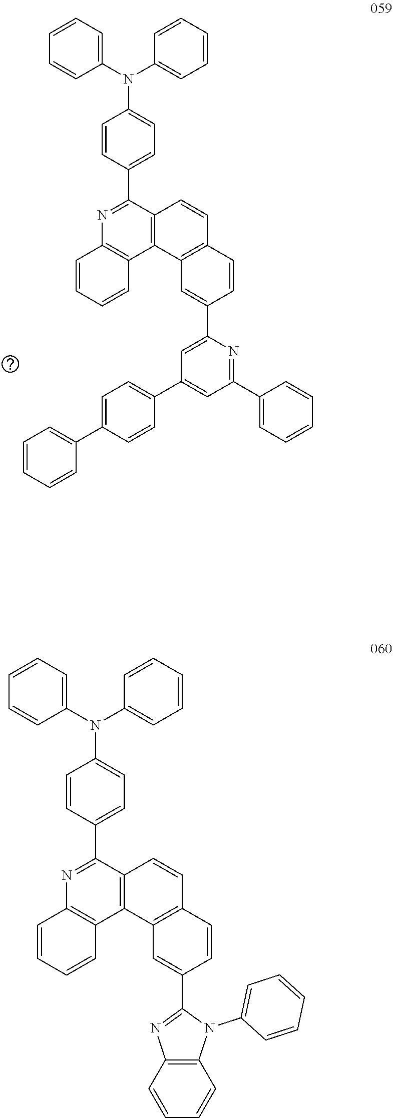 [SMILES: c1ccc(-c2ccc(-c3cc(-c4ccccc4)nc(-c4ccc5ccc6c(-c7ccc(N(c8ccccc8)c8ccccc8)cc7)nc7ccccc7c6c5c4)c3)cc2)cc1 ; c1ccc(N(c2ccccc2)c2ccc(-c3nc4ccccc4c4c3ccc3ccc(-c5nc6ccccc6n5-c5ccccc5)cc34)cc2)cc1]

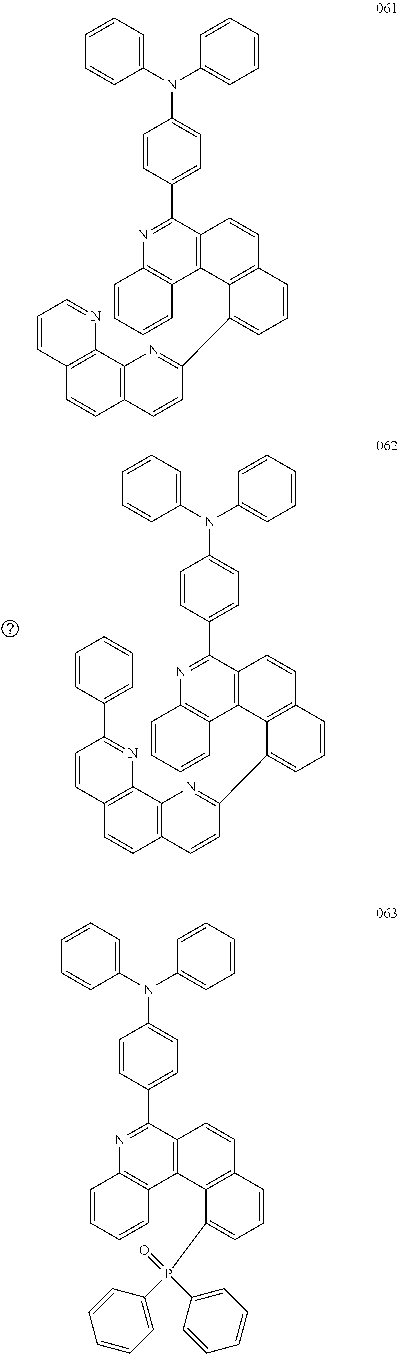

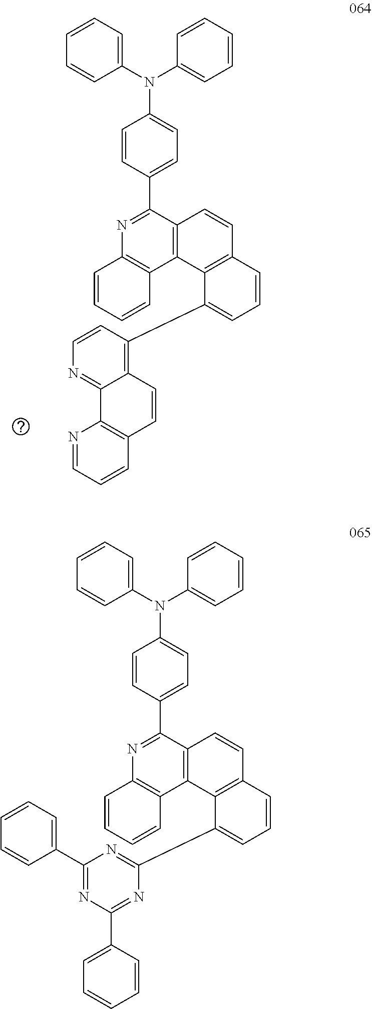 [SMILES: c1ccc(N(c2ccccc2)c2ccc(-c3nc4ccccc4c4c3ccc3cccc(-c5ccnc6c5ccc5cccnc56)c34)cc2)cc1 ; c1ccc(-c2nc(-c3ccccc3)nc(-c3cccc4ccc5c(-c6ccc(N(c7ccccc7)c7ccccc7)cc6)nc6ccccc6c5c34)n2)cc1]

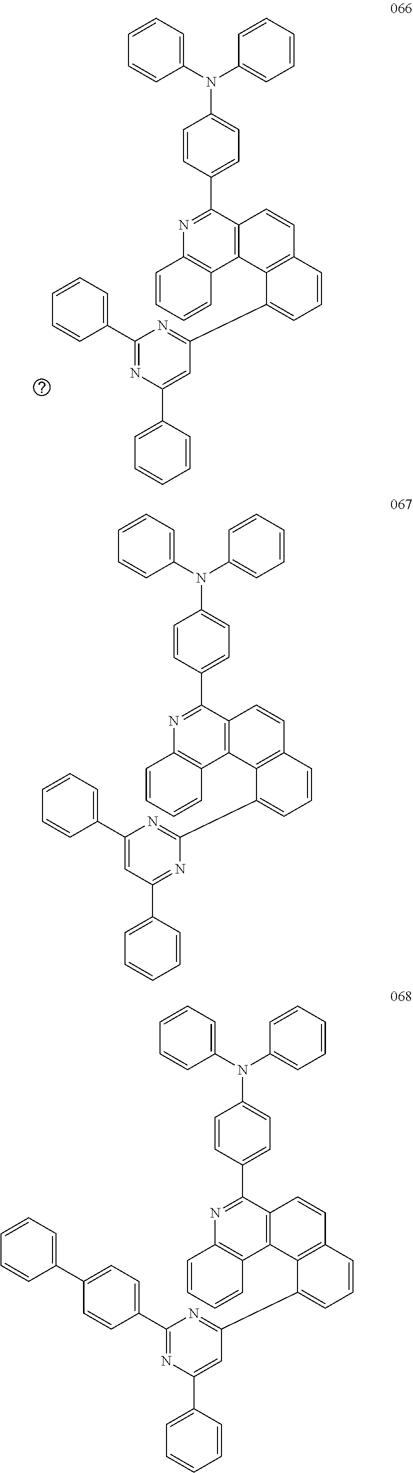

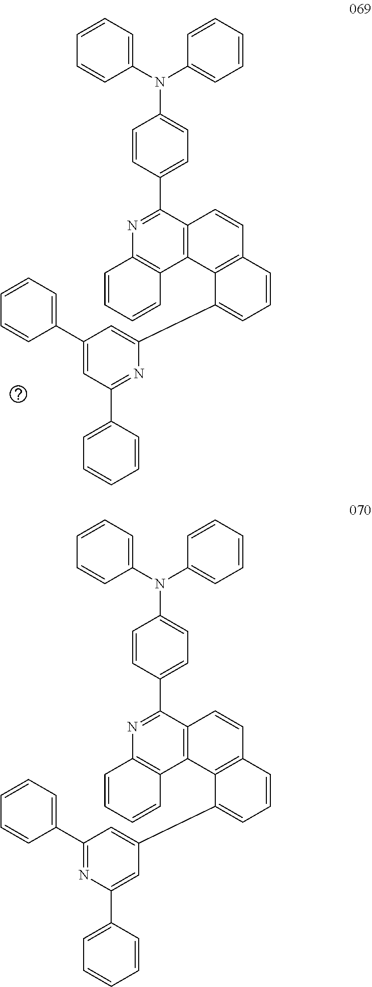

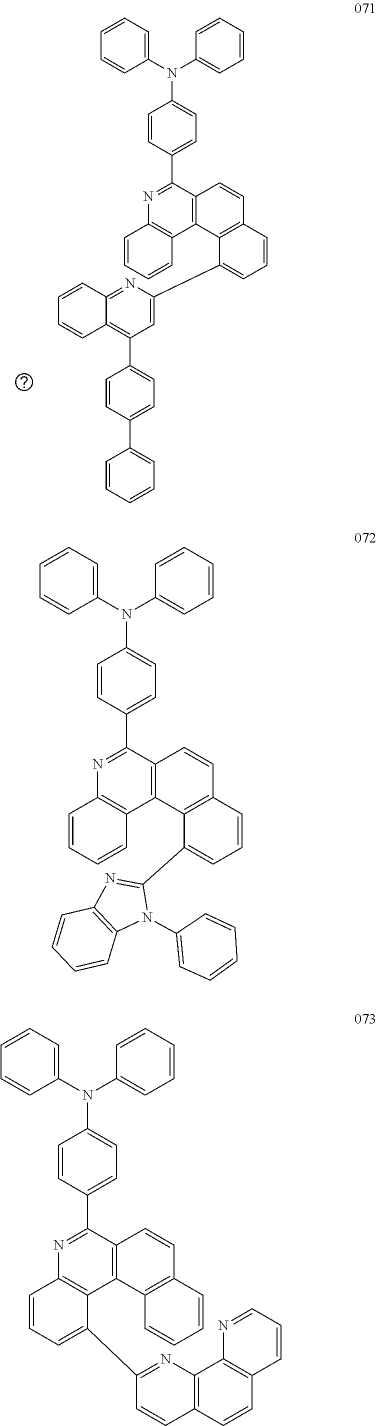 [SMILES: c1ccc(-c2ccc(-c3cc(-c4cccc5ccc6c(-c7ccc(N(c8ccccc8)c8ccccc8)cc7)nc7ccccc7c6c45)nc4ccccc34)cc2)cc1 ; c1ccc(N(c2ccccc2)c2ccc(-c3nc4ccccc4c4c3ccc3cccc(-c5nc6ccccc6n5-c5ccccc5)c34)cc2)cc1 ; c1ccc(N(c2ccccc2)c2ccc(-c3nc4cccc(-c5ccc6ccc7cccnc7c6n5)c4c4c3ccc3ccccc34)cc2)cc1]

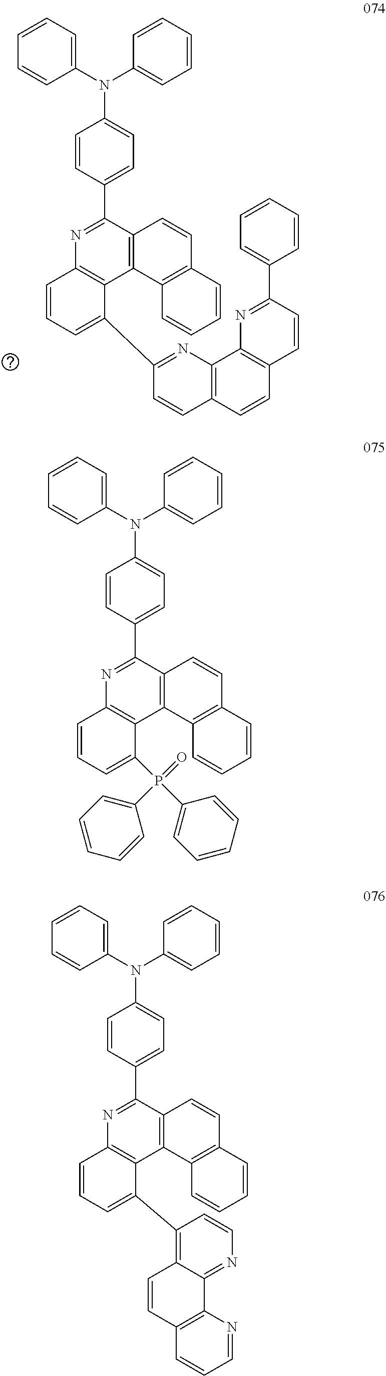

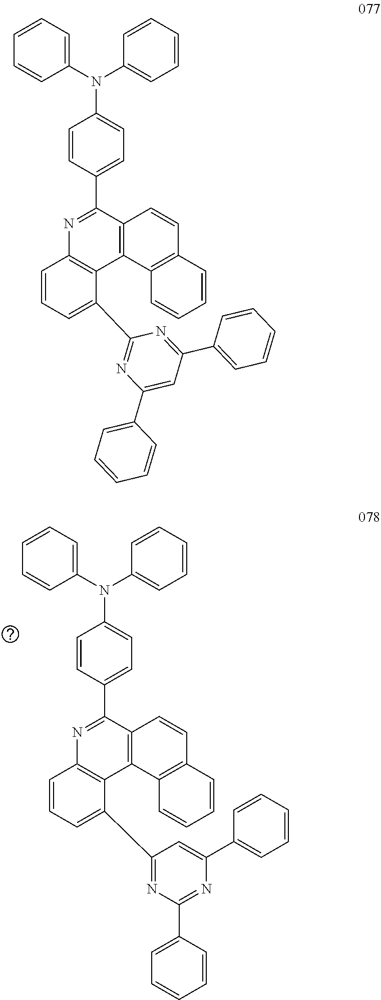 [SMILES: c1ccc(-c2cc(-c3ccccc3)nc(-c3cccc4nc(-c5ccc(N(c6ccccc6)c6ccccc6)cc5)c5ccc6ccccc6c5c34)n2)cc1 ; c1ccc(-c2cc(-c3cccc4nc(-c5ccc(N(c6ccccc6)c6ccccc6)cc5)c5ccc6ccccc6c5c34)nc(-c3ccccc3)n2)cc1]

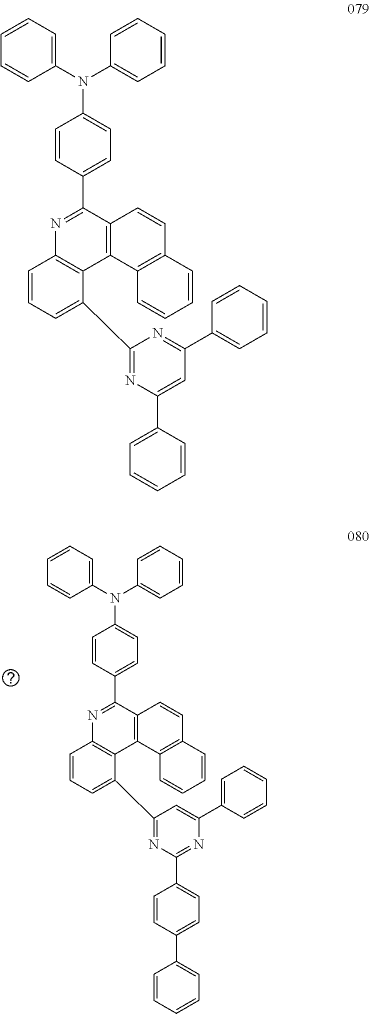 [SMILES: c1ccc(-c2cc(-c3ccccc3)nc(-c3cccc4nc(-c5ccc(N(c6ccccc6)c6ccccc6)cc5)c5ccc6ccccc6c5c34)n2)cc1 ; c1ccc(-c2ccc(-c3nc(-c4ccccc4)cc(-c4cccc5nc(-c6ccc(N(c7ccccc7)c7ccccc7)cc6)c6ccc7ccccc7c6c45)n3)cc2)cc1]

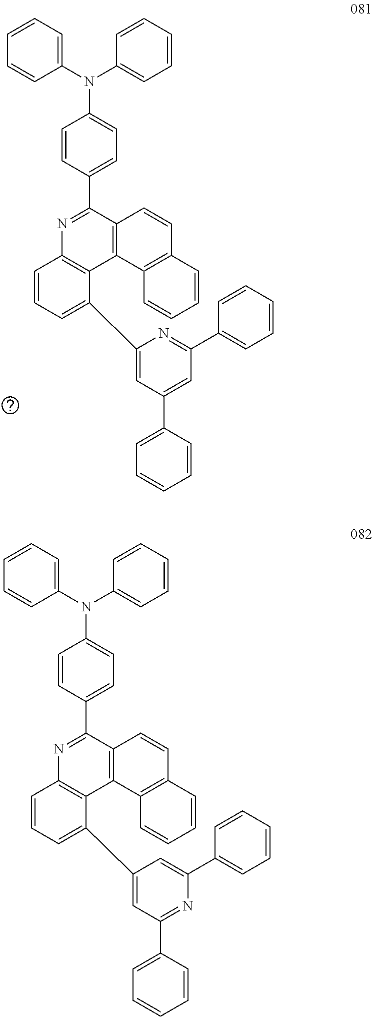 [SMILES: c1ccc(-c2cc(-c3ccccc3)nc(-c3cccc4nc(-c5ccc(N(c6ccccc6)c6ccccc6)cc5)c5ccc6ccccc6c5c34)c2)cc1 ; c1ccc(-c2cc(-c3cccc4nc(-c5ccc(N(c6ccccc6)c6ccccc6)cc5)c5ccc6ccccc6c5c34)cc(-c3ccccc3)n2)cc1]

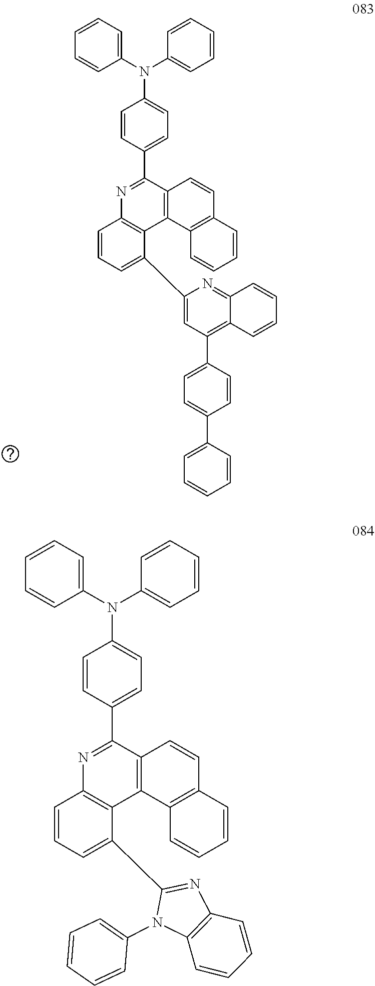 [SMILES: c1ccc(-c2ccc(-c3cc(-c4cccc5nc(-c6ccc(N(c7ccccc7)c7ccccc7)cc6)c6ccc7ccccc7c6c45)nc4ccccc34)cc2)cc1 ; c1ccc(N(c2ccccc2)c2ccc(-c3nc4cccc(-c5nc6ccccc6n5-c5ccccc5)c4c4c3ccc3ccccc34)cc2)cc1]

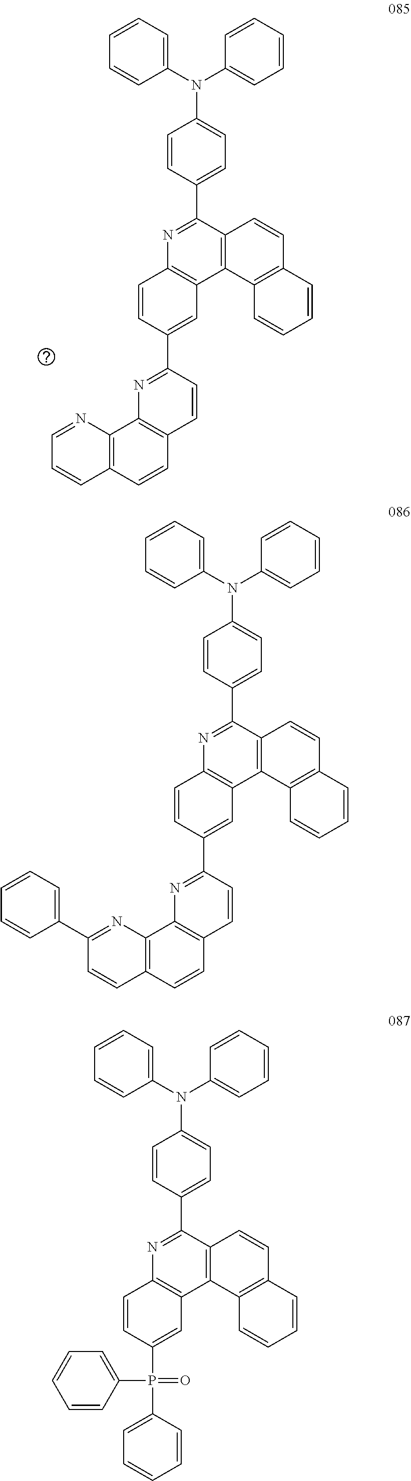

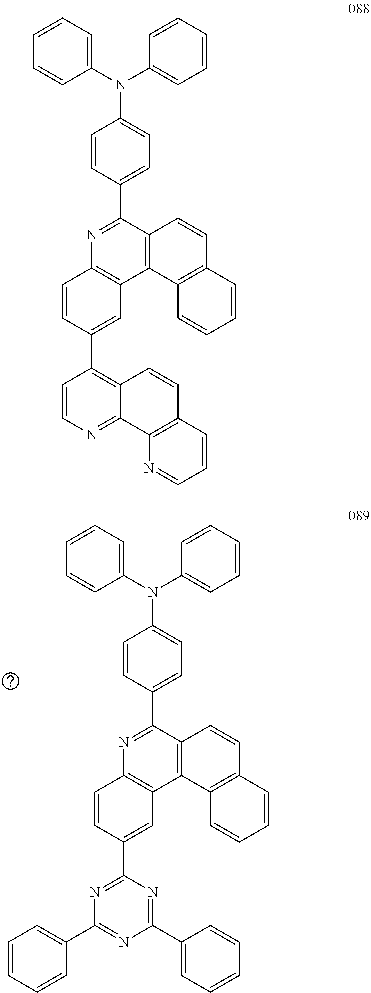 [SMILES: c1ccc(N(c2ccccc2)c2ccc(-c3nc4ccc(-c5ccnc6c5ccc5cccnc56)cc4c4c3ccc3ccccc34)cc2)cc1 ; c1ccc(-c2nc(-c3ccccc3)nc(-c3ccc4nc(-c5ccc(N(c6ccccc6)c6ccccc6)cc5)c5ccc6ccccc6c5c4c3)n2)cc1]

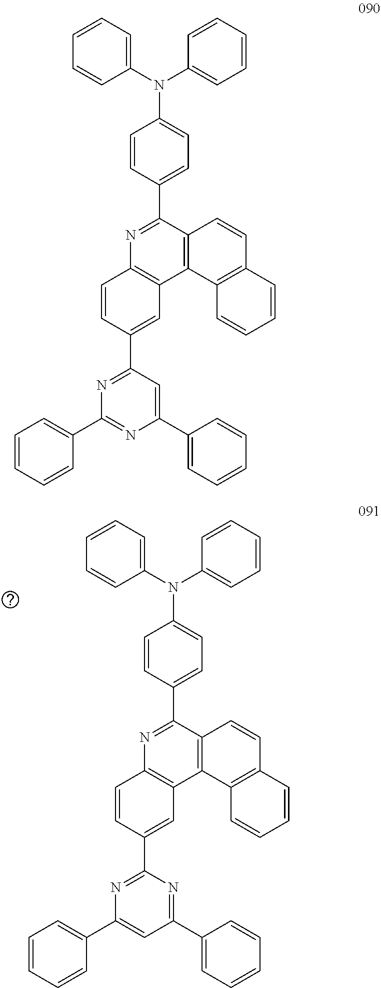

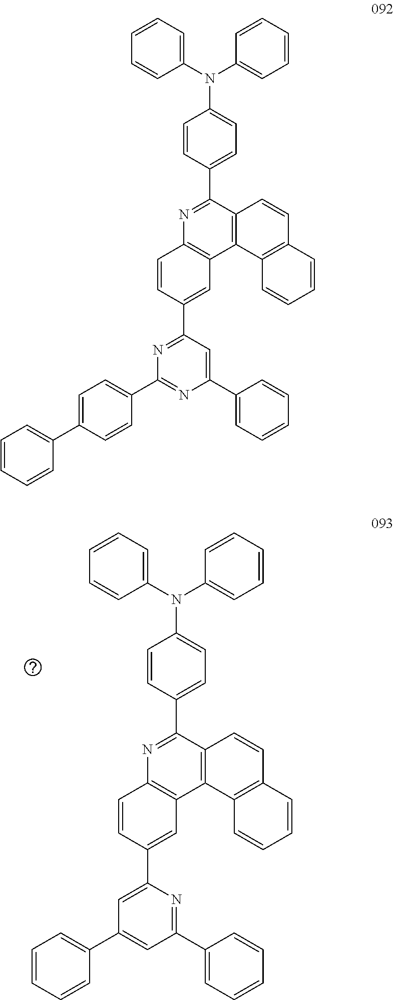 [SMILES: c1ccc(-c2ccc(-c3nc(-c4ccccc4)cc(-c4ccc5nc(-c6ccc(N(c7ccccc7)c7ccccc7)cc6)c6ccc7ccccc7c6c5c4)n3)cc2)cc1 ; c1ccc(-c2cc(-c3ccccc3)nc(-c3ccc4nc(-c5ccc(N(c6ccccc6)c6ccccc6)cc5)c5ccc6ccccc6c5c4c3)c2)cc1]

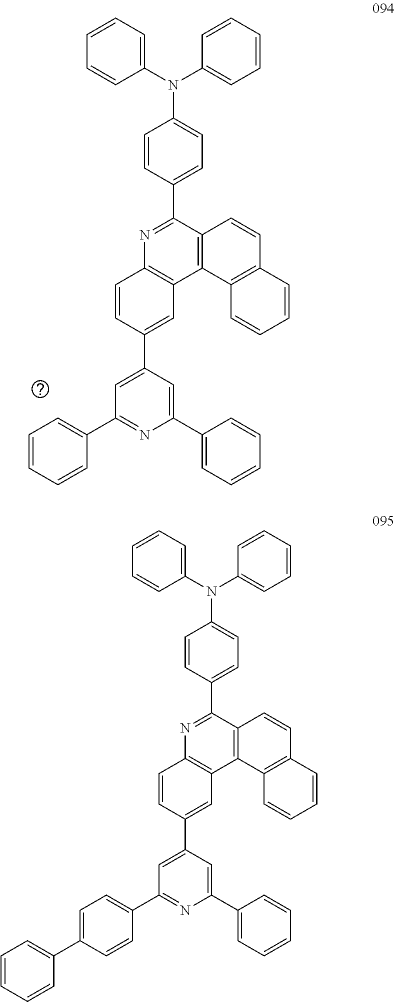 [SMILES: c1ccc(-c2cc(-c3ccc4nc(-c5ccc(N(c6ccccc6)c6ccccc6)cc5)c5ccc6ccccc6c5c4c3)cc(-c3ccccc3)n2)cc1 ; c1ccc(-c2ccc(-c3cc(-c4ccc5nc(-c6ccc(N(c7ccccc7)c7ccccc7)cc6)c6ccc7ccccc7c6c5c4)cc(-c4ccccc4)n3)cc2)cc1]

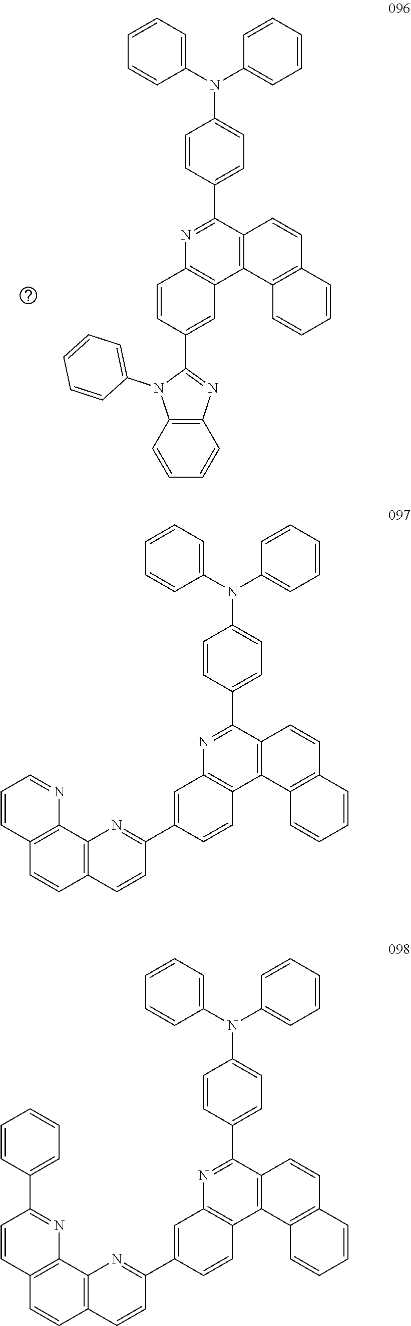 [SMILES: c1ccc(N(c2ccccc2)c2ccc(-c3nc4ccc(-c5nc6ccccc6n5-c5ccccc5)cc4c4c3ccc3ccccc34)cc2)cc1 ; c1ccc(N(c2ccccc2)c2ccc(-c3nc4cc(-c5ccc6ccc7cccnc7c6n5)ccc4c4c3ccc3ccccc34)cc2)cc1 ; c1ccc(-c2ccc3ccc4ccc(-c5ccc6c(c5)nc(-c5ccc(N(c7ccccc7)c7ccccc7)cc5)c5ccc7ccccc7c56)nc4c3n2)cc1]

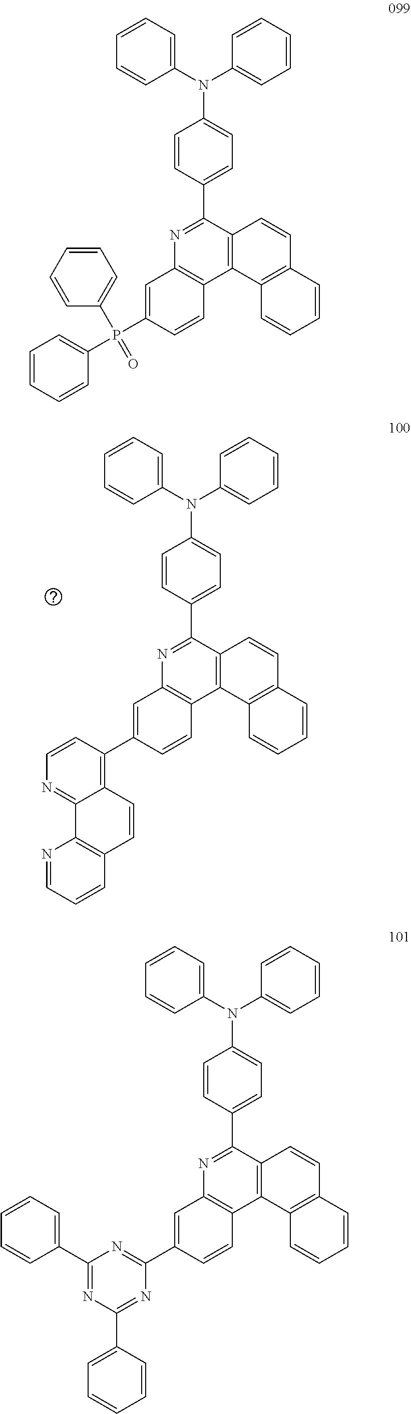 [SMILES: O=P(c1ccccc1)(c1ccccc1)c1ccc2c(c1)nc(-c1ccc(N(c3ccccc3)c3ccccc3)cc1)c1ccc3ccccc3c12 ; c1ccc(N(c2ccccc2)c2ccc(-c3nc4cc(-c5ccnc6c5ccc5cccnc56)ccc4c4c3ccc3ccccc34)cc2)cc1 ; c1ccc(-c2nc(-c3ccccc3)nc(-c3ccc4c(c3)nc(-c3ccc(N(c5ccccc5)c5ccccc5)cc3)c3ccc5ccccc5c34)n2)cc1]

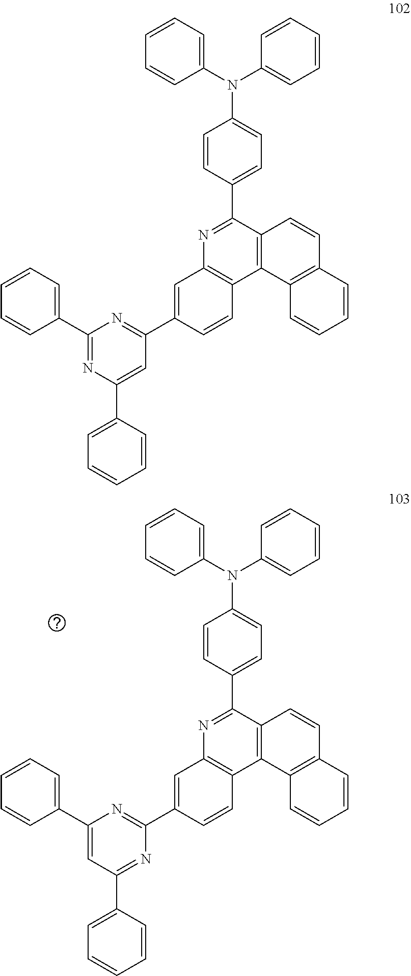 [SMILES: c1ccc(-c2cc(-c3ccc4c(c3)nc(-c3ccc(N(c5ccccc5)c5ccccc5)cc3)c3ccc5ccccc5c34)nc(-c3ccccc3)n2)cc1 ; c1ccc(-c2cc(-c3ccccc3)nc(-c3ccc4c(c3)nc(-c3ccc(N(c5ccccc5)c5ccccc5)cc3)c3ccc5ccccc5c34)n2)cc1]

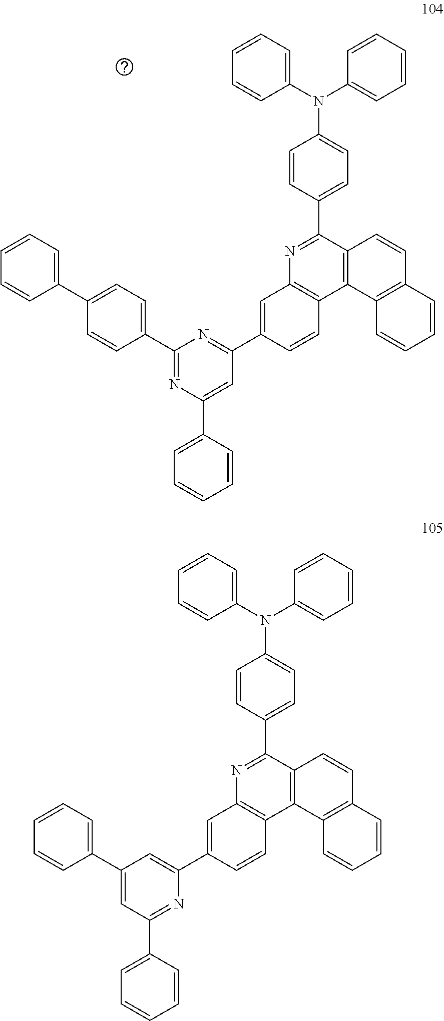 [SMILES: c1ccc(-c2ccc(-c3nc(-c4ccccc4)cc(-c4ccc5c(c4)nc(-c4ccc(N(c6ccccc6)c6ccccc6)cc4)c4ccc6ccccc6c45)n3)cc2)cc1 ; c1ccc(-c2cc(-c3ccccc3)nc(-c3ccc4c(c3)nc(-c3ccc(N(c5ccccc5)c5ccccc5)cc3)c3ccc5ccccc5c34)c2)cc1]

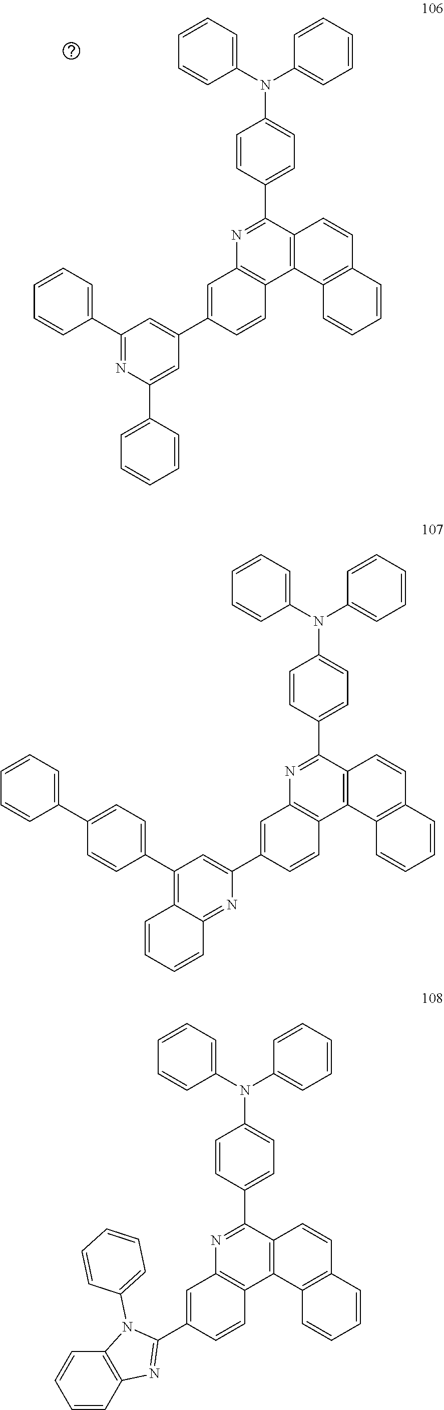 [SMILES: c1ccc(-c2cc(-c3ccc4c(c3)nc(-c3ccc(N(c5ccccc5)c5ccccc5)cc3)c3ccc5ccccc5c34)cc(-c3ccccc3)n2)cc1 ; c1ccc(-c2ccc(-c3cc(-c4ccc5c(c4)nc(-c4ccc(N(c6ccccc6)c6ccccc6)cc4)c4ccc6ccccc6c45)nc4ccccc34)cc2)cc1 ; c1ccc(N(c2ccccc2)c2ccc(-c3nc4cc(-c5nc6ccccc6n5-c5ccccc5)ccc4c4c3ccc3ccccc34)cc2)cc1]

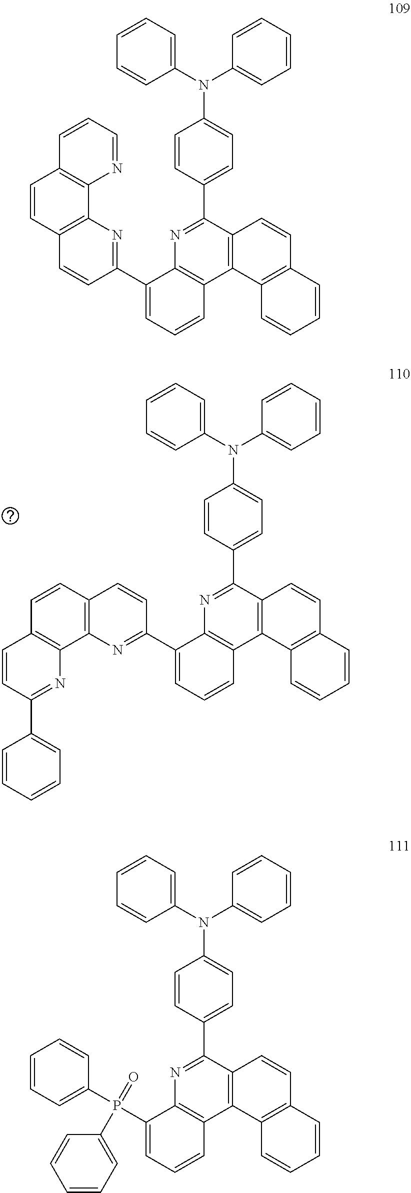 [SMILES: c1ccc(N(c2ccccc2)c2ccc(-c3nc4c(-c5ccc6ccc7cccnc7c6n5)cccc4c4c3ccc3ccccc34)cc2)cc1 ; c1ccc(-c2ccc3ccc4ccc(-c5cccc6c5nc(-c5ccc(N(c7ccccc7)c7ccccc7)cc5)c5ccc7ccccc7c56)nc4c3n2)cc1 ; O=P(c1ccccc1)(c1ccccc1)c1cccc2c1nc(-c1ccc(N(c3ccccc3)c3ccccc3)cc1)c1ccc3ccccc3c12]

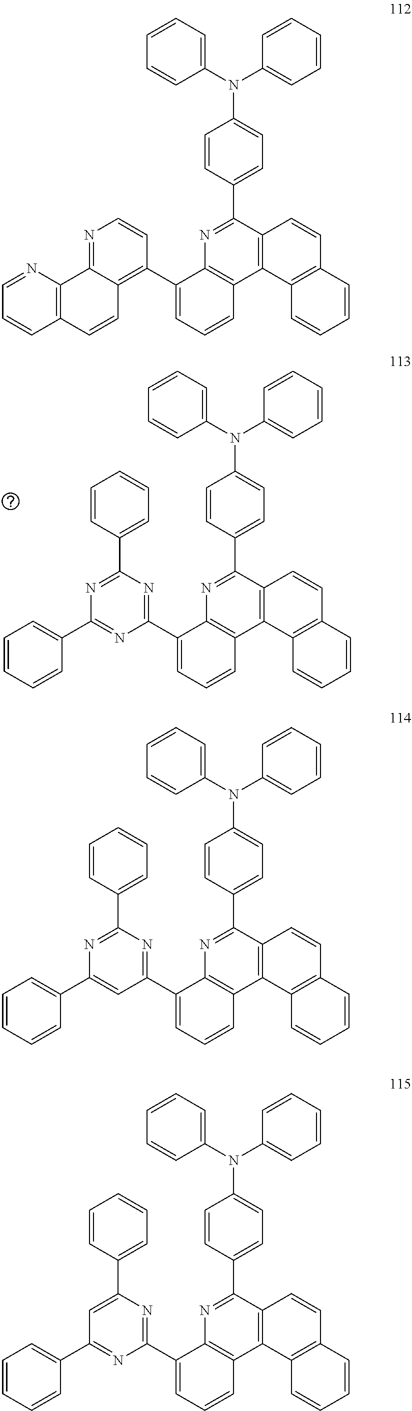

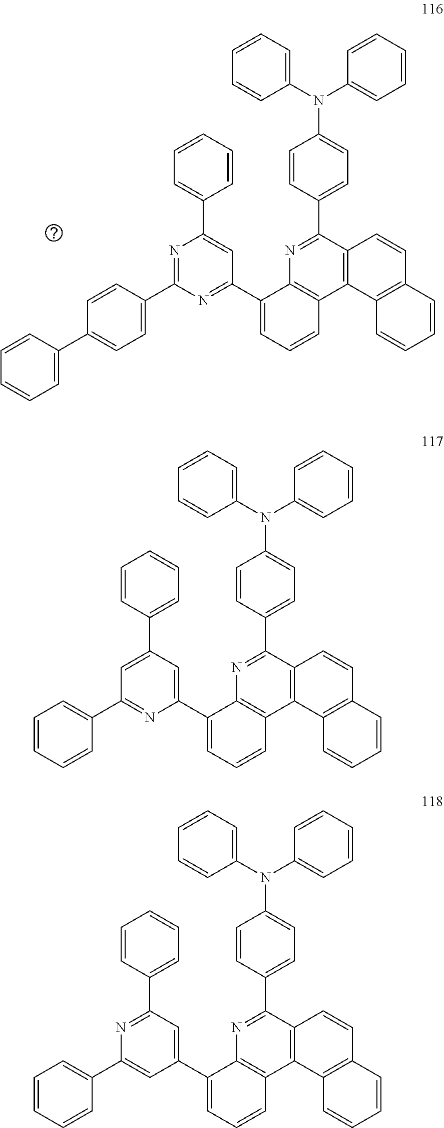 [SMILES: c1ccc(-c2ccc(-c3nc(-c4ccccc4)cc(-c4cccc5c4nc(-c4ccc(N(c6ccccc6)c6ccccc6)cc4)c4ccc6ccccc6c45)n3)cc2)cc1 ; c1ccc(-c2cc(-c3ccccc3)nc(-c3cccc4c3nc(-c3ccc(N(c5ccccc5)c5ccccc5)cc3)c3ccc5ccccc5c34)c2)cc1 ; c1ccc(-c2cc(-c3cccc4c3nc(-c3ccc(N(c5ccccc5)c5ccccc5)cc3)c3ccc5ccccc5c34)cc(-c3ccccc3)n2)cc1]

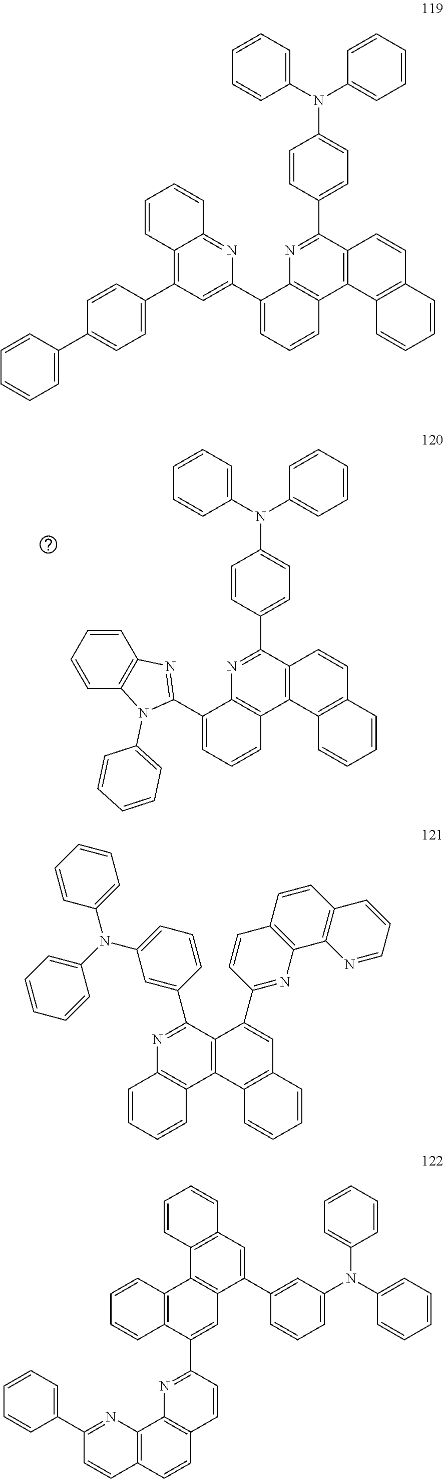

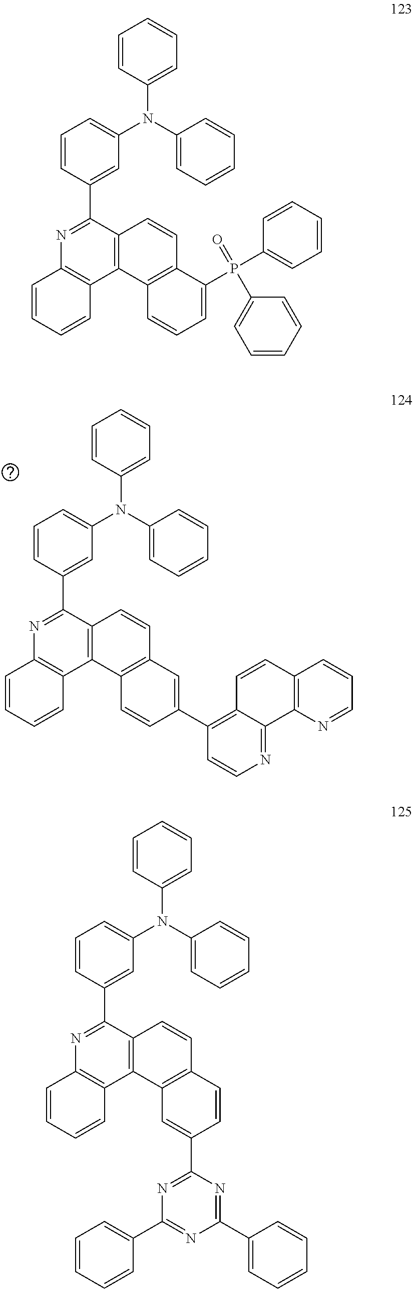 [SMILES: O=P(c1ccccc1)(c1ccccc1)c1cccc2c1ccc1c(-c3cccc(N(c4ccccc4)c4ccccc4)c3)nc3ccccc3c12 ; c1ccc(N(c2ccccc2)c2cccc(-c3nc4ccccc4c4c3ccc3cc(-c5ccnc6c5ccc5cccnc56)ccc34)c2)cc1 ; c1ccc(-c2nc(-c3ccccc3)nc(-c3ccc4ccc5c(-c6cccc(N(c7ccccc7)c7ccccc7)c6)nc6ccccc6c5c4c3)n2)cc1]

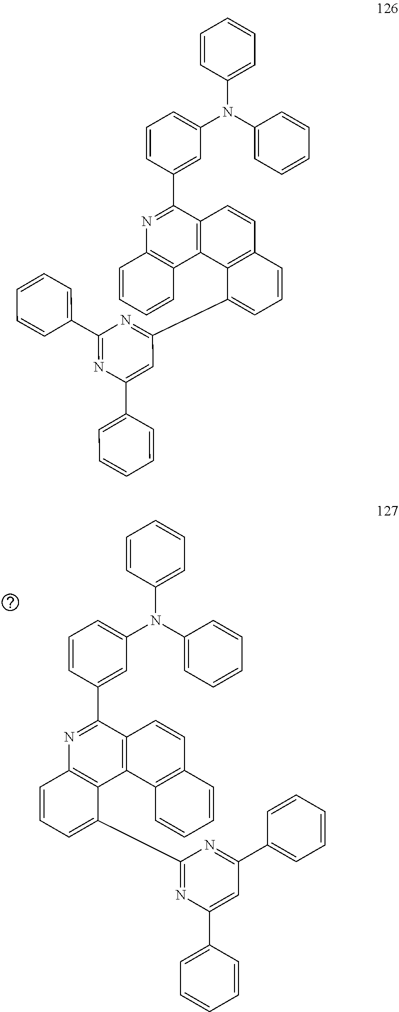 [SMILES: c1ccc(-c2cc(-c3cccc4ccc5c(-c6cccc(N(c7ccccc7)c7ccccc7)c6)nc6ccccc6c5c34)nc(-c3ccccc3)n2)cc1 ; c1ccc(-c2cc(-c3ccccc3)nc(-c3cccc4nc(-c5cccc(N(c6ccccc6)c6ccccc6)c5)c5ccc6ccccc6c5c34)n2)cc1]

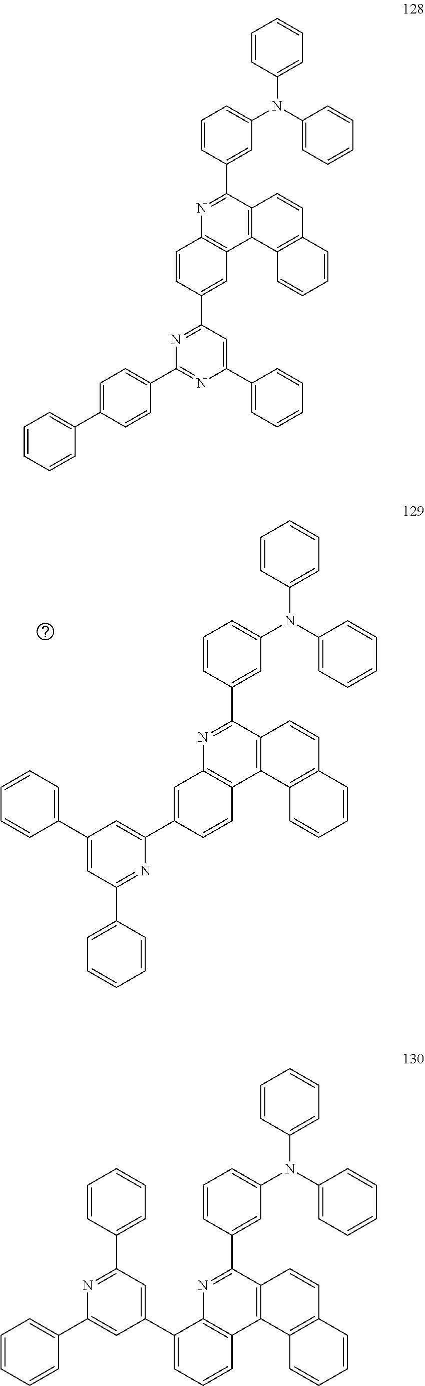 [SMILES: c1ccc(-c2ccc(-c3nc(-c4ccccc4)cc(-c4ccc5nc(-c6cccc(N(c7ccccc7)c7ccccc7)c6)c6ccc7ccccc7c6c5c4)n3)cc2)cc1 ; c1ccc(-c2cc(-c3ccccc3)nc(-c3ccc4c(c3)nc(-c3cccc(N(c5ccccc5)c5ccccc5)c3)c3ccc5ccccc5c34)c2)cc1 ; c1ccc(-c2cc(-c3cccc4c3nc(-c3cccc(N(c5ccccc5)c5ccccc5)c3)c3ccc5ccccc5c34)cc(-c3ccccc3)n2)cc1]

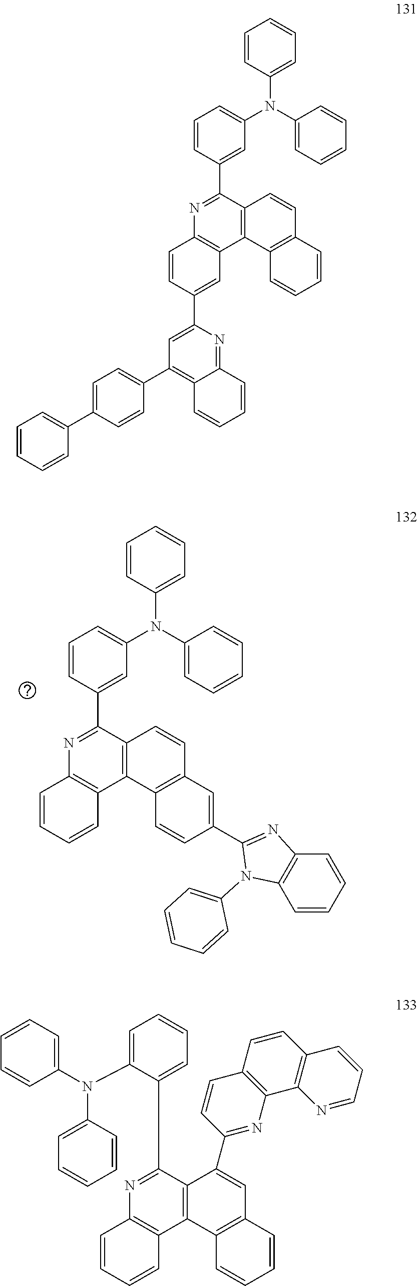 [SMILES: c1ccc(-c2ccc(-c3cc(-c4ccc5nc(-c6cccc(N(c7ccccc7)c7ccccc7)c6)c6ccc7ccccc7c6c5c4)nc4ccccc34)cc2)cc1 ; c1ccc(N(c2ccccc2)c2cccc(-c3nc4ccccc4c4c3ccc3cc(-c5nc6ccccc6n5-c5ccccc5)ccc34)c2)cc1 ; c1ccc(N(c2ccccc2)c2ccccc2-c2nc3ccccc3c3c2c(-c2ccc4ccc5cccnc5c4n2)cc2ccccc23)cc1]

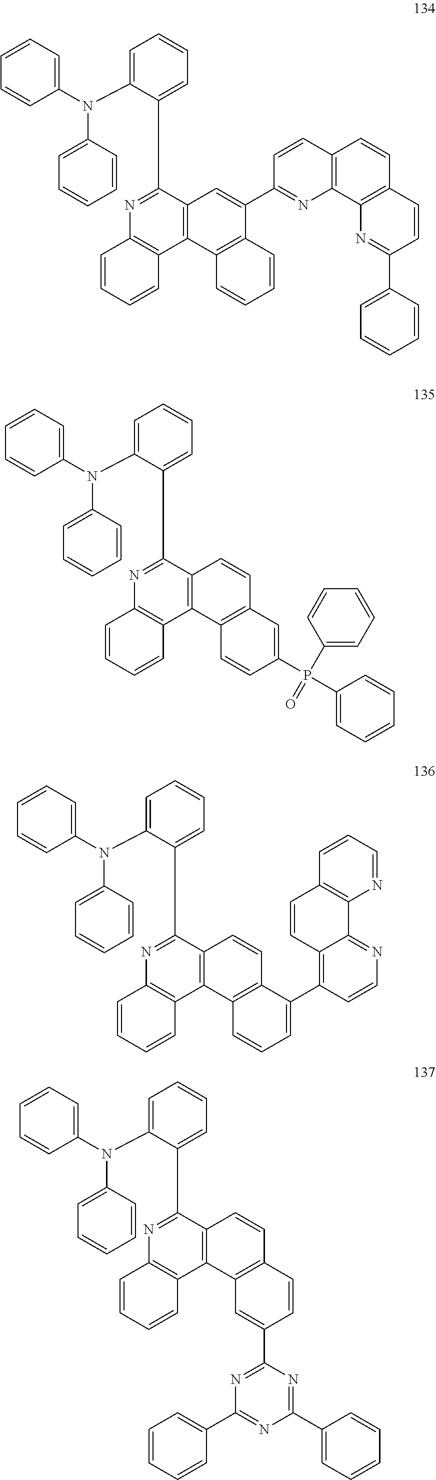

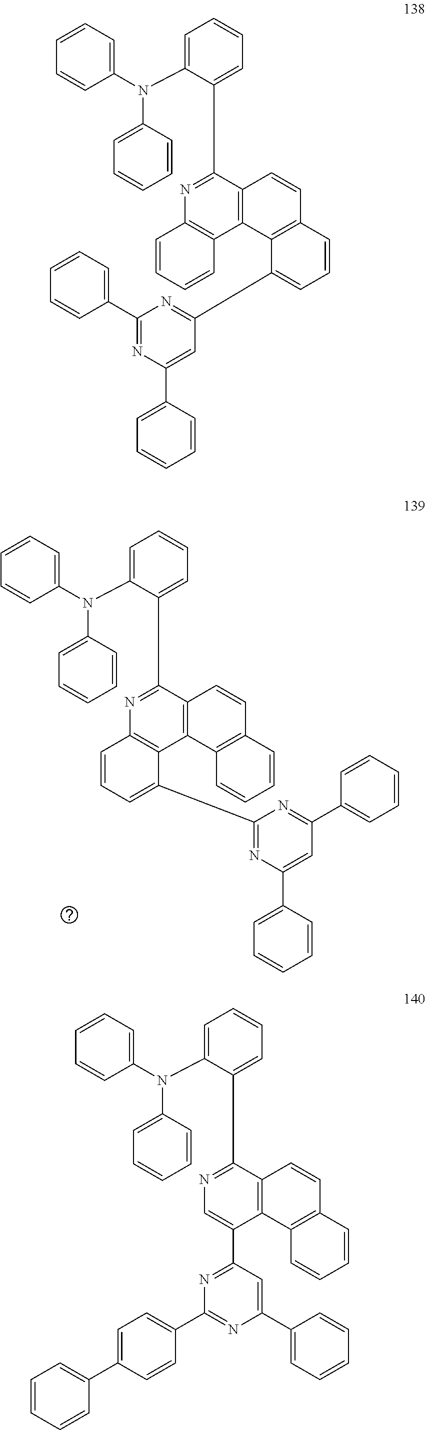 [SMILES: c1ccc(-c2cc(-c3cccc4ccc5c(-c6ccccc6N(c6ccccc6)c6ccccc6)nc6ccccc6c5c34)nc(-c3ccccc3)n2)cc1 ; c1ccc(-c2cc(-c3ccccc3)nc(-c3cccc4nc(-c5ccccc5N(c5ccccc5)c5ccccc5)c5ccc6ccccc6c5c34)n2)cc1 ; c1ccc(-c2ccc(-c3nc(-c4ccccc4)cc(-c4cnc(-c5ccccc5N(c5ccccc5)c5ccccc5)c5ccc6ccccc6c45)n3)cc2)cc1]

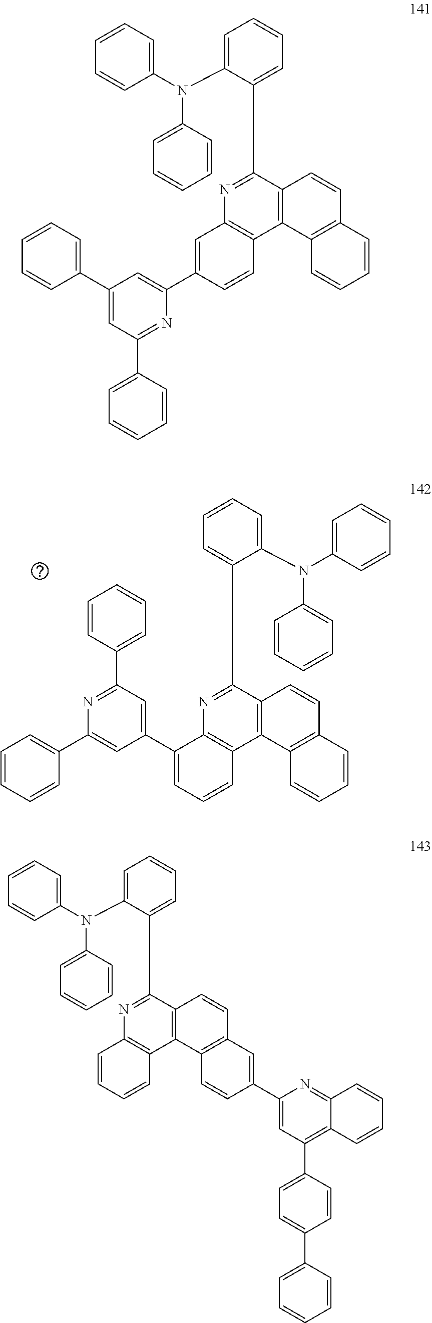 [SMILES: c1ccc(-c2cc(-c3ccccc3)nc(-c3ccc4c(c3)nc(-c3ccccc3N(c3ccccc3)c3ccccc3)c3ccc5ccccc5c34)c2)cc1 ; c1ccc(-c2cc(-c3cccc4c3nc(-c3ccccc3N(c3ccccc3)c3ccccc3)c3ccc5ccccc5c34)cc(-c3ccccc3)n2)cc1 ; c1ccc(-c2ccc(-c3cc(-c4ccc5c(ccc6c(-c7ccccc7N(c7ccccc7)c7ccccc7)nc7ccccc7c65)c4)nc4ccccc34)cc2)cc1]

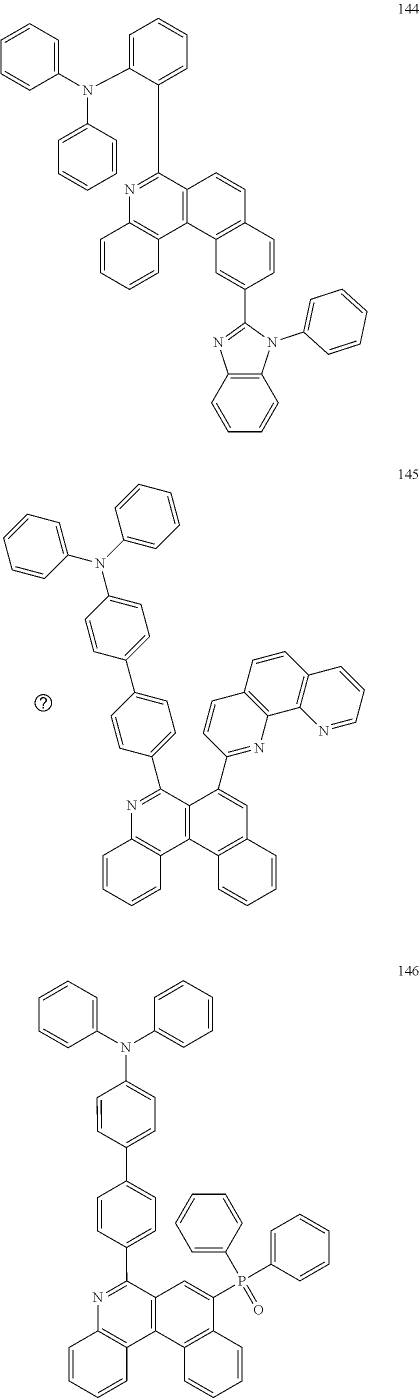 [SMILES: c1ccc(N(c2ccccc2)c2ccccc2-c2nc3ccccc3c3c2ccc2ccc(-c4nc5ccccc5n4-c4ccccc4)cc23)cc1 ; c1ccc(N(c2ccccc2)c2ccc(-c3ccc(-c4nc5ccccc5c5c4c(-c4ccc6ccc7cccnc7c6n4)cc4ccccc45)cc3)cc2)cc1 ; O=P(c1ccccc1)(c1ccccc1)c1cc2c(-c3ccc(-c4ccc(N(c5ccccc5)c5ccccc5)cc4)cc3)nc3ccccc3c2c2ccccc12]

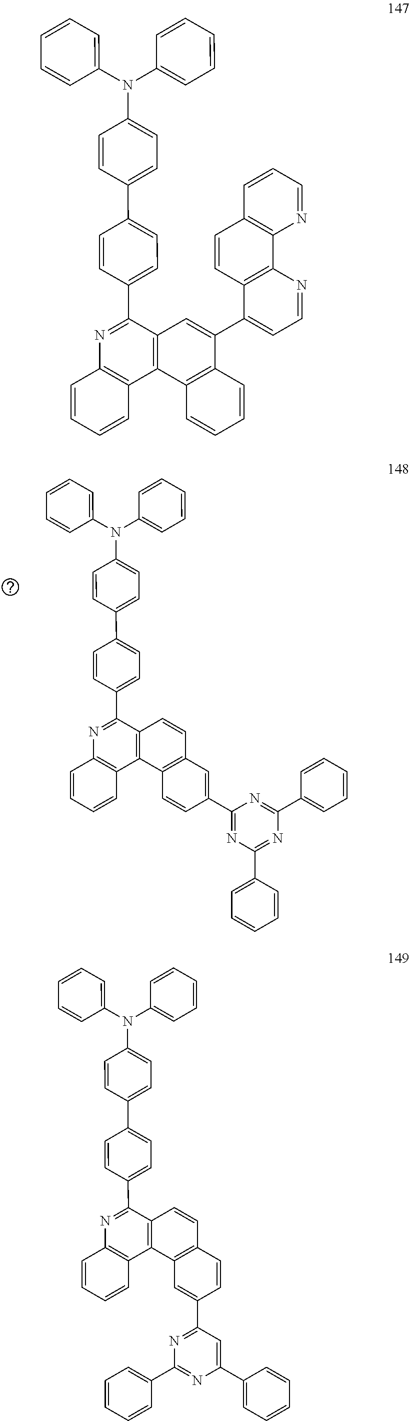 [SMILES: c1ccc(N(c2ccccc2)c2ccc(-c3ccc(-c4nc5ccccc5c5c4cc(-c4ccnc6c4ccc4cccnc46)c4ccccc45)cc3)cc2)cc1 ; c1ccc(-c2nc(-c3ccccc3)nc(-c3ccc4c(ccc5c(-c6ccc(-c7ccc(N(c8ccccc8)c8ccccc8)cc7)cc6)nc6ccccc6c54)c3)n2)cc1 ; c1ccc(-c2cc(-c3ccc4ccc5c(-c6ccc(-c7ccc(N(c8ccccc8)c8ccccc8)cc7)cc6)nc6ccccc6c5c4c3)nc(-c3ccccc3)n2)cc1]

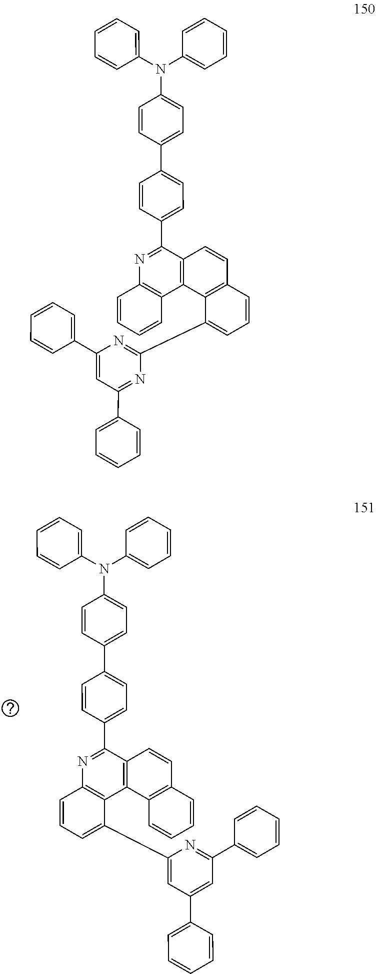 [SMILES: c1ccc(-c2cc(-c3ccccc3)nc(-c3cccc4ccc5c(-c6ccc(-c7ccc(N(c8ccccc8)c8ccccc8)cc7)cc6)nc6ccccc6c5c34)n2)cc1 ; c1ccc(-c2cc(-c3ccccc3)nc(-c3cccc4nc(-c5ccc(-c6ccc(N(c7ccccc7)c7ccccc7)cc6)cc5)c5ccc6ccccc6c5c34)c2)cc1]

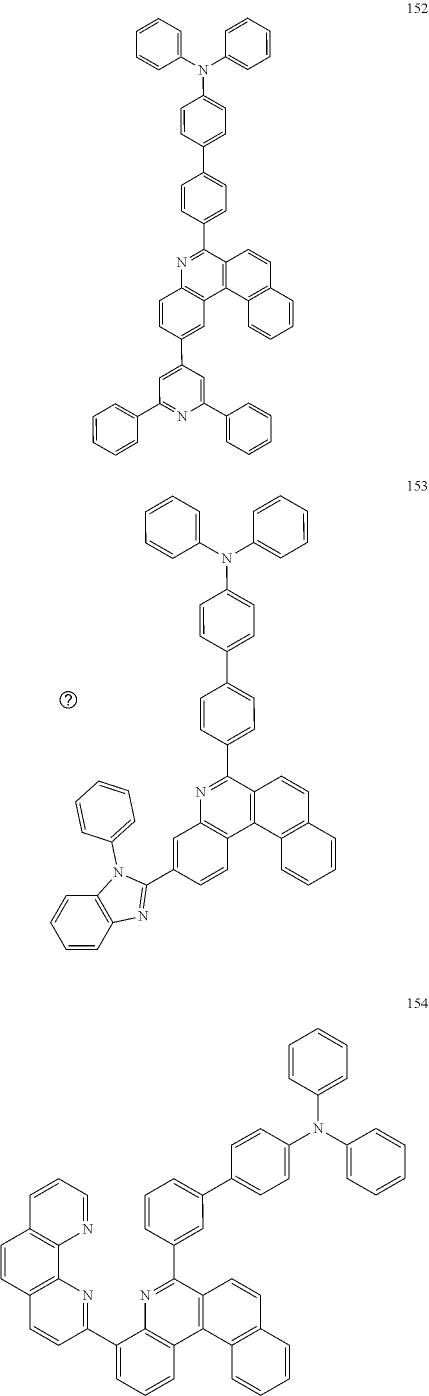 [SMILES: c1ccc(-c2cc(-c3ccc4nc(-c5ccc(-c6ccc(N(c7ccccc7)c7ccccc7)cc6)cc5)c5ccc6ccccc6c5c4c3)cc(-c3ccccc3)n2)cc1 ; c1ccc(N(c2ccccc2)c2ccc(-c3ccc(-c4nc5cc(-c6nc7ccccc7n6-c6ccccc6)ccc5c5c4ccc4ccccc45)cc3)cc2)cc1 ; c1ccc(N(c2ccccc2)c2ccc(-c3cccc(-c4nc5c(-c6ccc7ccc8cccnc8c7n6)cccc5c5c4ccc4ccccc45)c3)cc2)cc1]

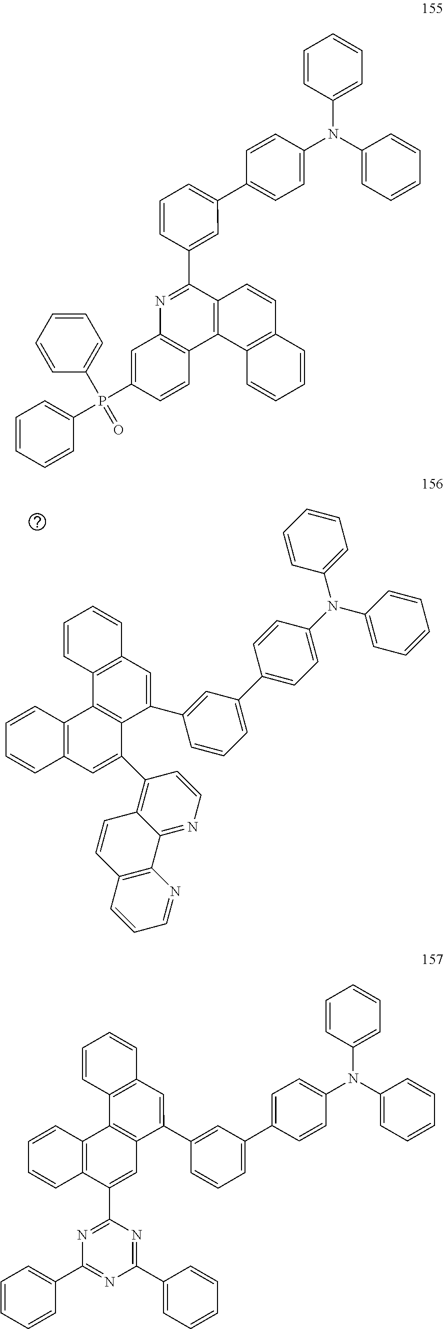 [SMILES: O=P(c1ccccc1)(c1ccccc1)c1ccc2c(c1)nc(-c1cccc(-c3ccc(N(c4ccccc4)c4ccccc4)cc3)c1)c1ccc3ccccc3c12 ; c1ccc(N(c2ccccc2)c2ccc(-c3cccc(-c4cc5ccccc5c5c4c(-c4ccnc6c4ccc4cccnc46)cc4ccccc45)c3)cc2)cc1 ; c1ccc(-c2nc(-c3ccccc3)nc(-c3cc4c(-c5cccc(-c6ccc(N(c7ccccc7)c7ccccc7)cc6)c5)cc5ccccc5c4c4ccccc34)n2)cc1]

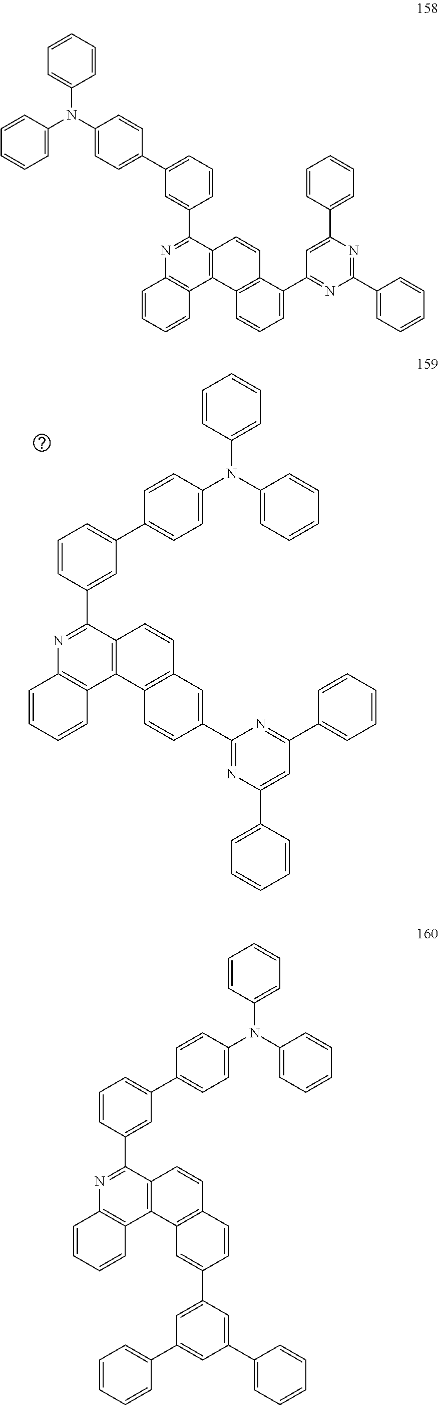 [SMILES: c1ccc(-c2cc(-c3cccc4c3ccc3c(-c5cccc(-c6ccc(N(c7ccccc7)c7ccccc7)cc6)c5)nc5ccccc5c34)nc(-c3ccccc3)n2)cc1 ; c1ccc(-c2cc(-c3ccccc3)nc(-c3ccc4c(ccc5c(-c6cccc(-c7ccc(N(c8ccccc8)c8ccccc8)cc7)c6)nc6ccccc6c54)c3)n2)cc1 ; c1ccc(-c2cc(-c3ccccc3)cc(-c3ccc4ccc5c(-c6cccc(-c7ccc(N(c8ccccc8)c8ccccc8)cc7)c6)nc6ccccc6c5c4c3)c2)cc1]

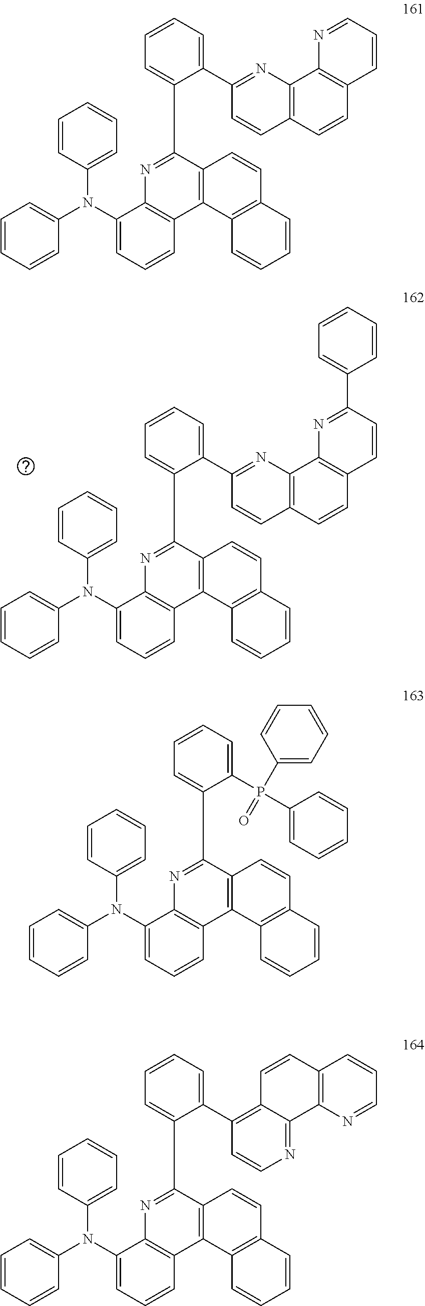 [SMILES: c1ccc(N(c2ccccc2)c2cccc3c2nc(-c2ccccc2-c2ccc4ccc5cccnc5c4n2)c2ccc4ccccc4c23)cc1 ; c1ccc(-c2ccc3ccc4ccc(-c5ccccc5-c5nc6c(N(c7ccccc7)c7ccccc7)cccc6c6c5ccc5ccccc56)nc4c3n2)cc1 ; O=P(c1ccccc1)(c1ccccc1)c1ccccc1-c1nc2c(N(c3ccccc3)c3ccccc3)cccc2c2c1ccc1ccccc12 ; c1ccc(N(c2ccccc2)c2cccc3c2nc(-c2ccccc2-c2ccnc4c2ccc2cccnc24)c2ccc4ccccc4c23)cc1]

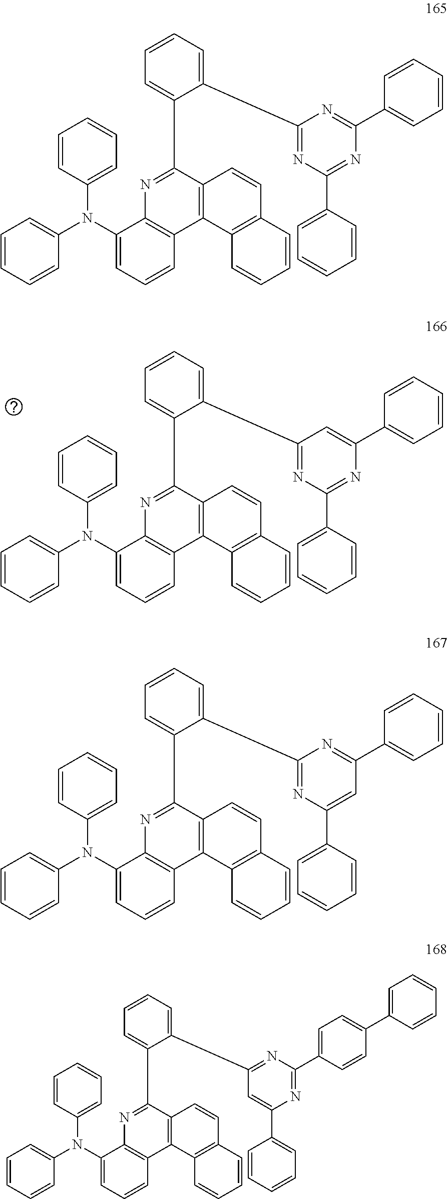 [SMILES: c1ccc(-c2nc(-c3ccccc3)nc(-c3ccccc3-c3nc4c(N(c5ccccc5)c5ccccc5)cccc4c4c3ccc3ccccc34)n2)cc1 ; c1ccc(-c2cc(-c3ccccc3-c3nc4c(N(c5ccccc5)c5ccccc5)cccc4c4c3ccc3ccccc34)nc(-c3ccccc3)n2)cc1 ; c1ccc(-c2cc(-c3ccccc3)nc(-c3ccccc3-c3nc4c(N(c5ccccc5)c5ccccc5)cccc4c4c3ccc3ccccc34)n2)cc1 ; c1ccc(-c2ccc(-c3nc(-c4ccccc4)cc(-c4ccccc4-c4nc5c(N(c6ccccc6)c6ccccc6)cccc5c5c4ccc4ccccc45)n3)cc2)cc1]

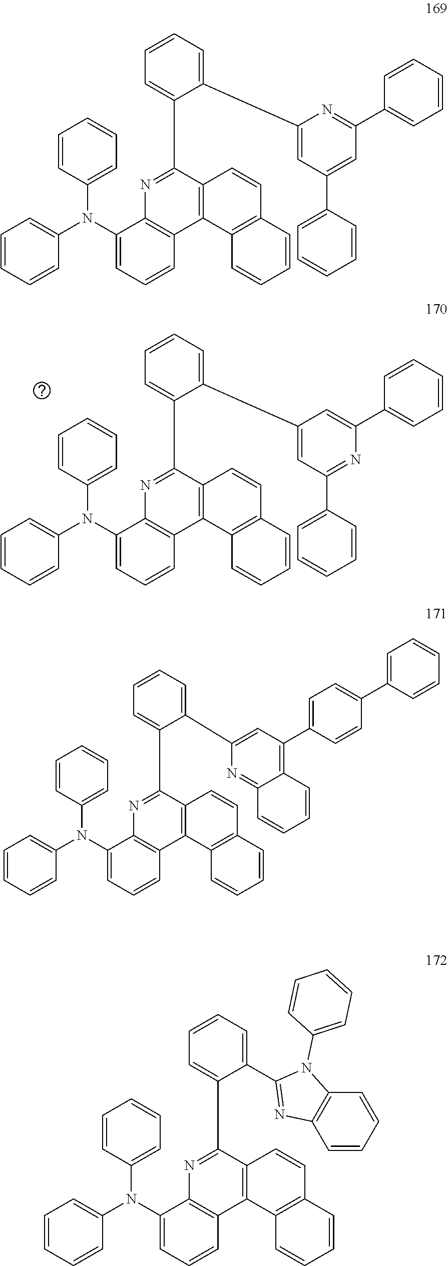 [SMILES: c1ccc(-c2cc(-c3ccccc3)nc(-c3ccccc3-c3nc4c(N(c5ccccc5)c5ccccc5)cccc4c4c3ccc3ccccc34)c2)cc1 ; c1ccc(-c2cc(-c3ccccc3-c3nc4c(N(c5ccccc5)c5ccccc5)cccc4c4c3ccc3ccccc34)cc(-c3ccccc3)n2)cc1 ; c1ccc(-c2ccc(-c3cc(-c4ccccc4-c4nc5c(N(c6ccccc6)c6ccccc6)cccc5c5c4ccc4ccccc45)nc4ccccc34)cc2)cc1 ; c1ccc(N(c2ccccc2)c2cccc3c2nc(-c2ccccc2-c2nc4ccccc4n2-c2ccccc2)c2ccc4ccccc4c23)cc1]

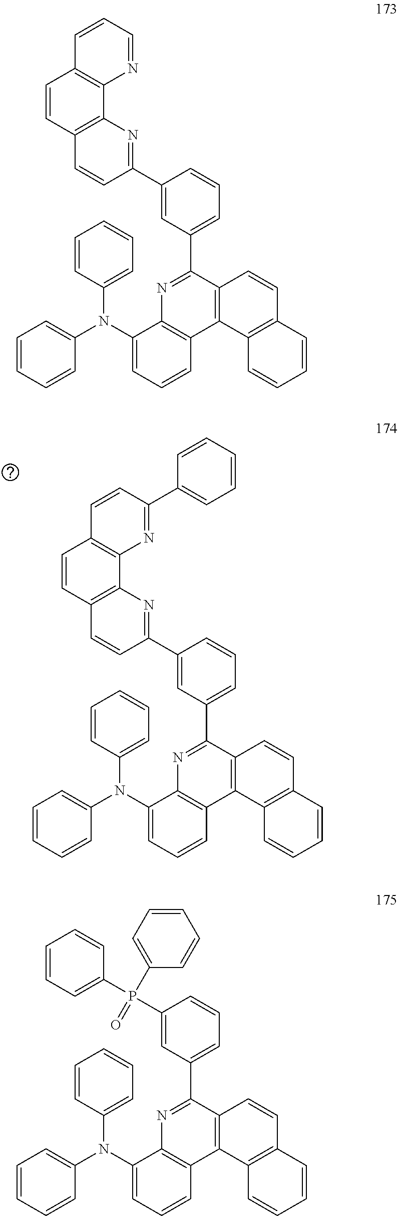 [SMILES: c1ccc(N(c2ccccc2)c2cccc3c2nc(-c2cccc(-c4ccc5ccc6cccnc6c5n4)c2)c2ccc4ccccc4c23)cc1 ; c1ccc(-c2ccc3ccc4ccc(-c5cccc(-c6nc7c(N(c8ccccc8)c8ccccc8)cccc7c7c6ccc6ccccc67)c5)nc4c3n2)cc1 ; O=P(c1ccccc1)(c1ccccc1)c1cccc(-c2nc3c(N(c4ccccc4)c4ccccc4)cccc3c3c2ccc2ccccc23)c1]

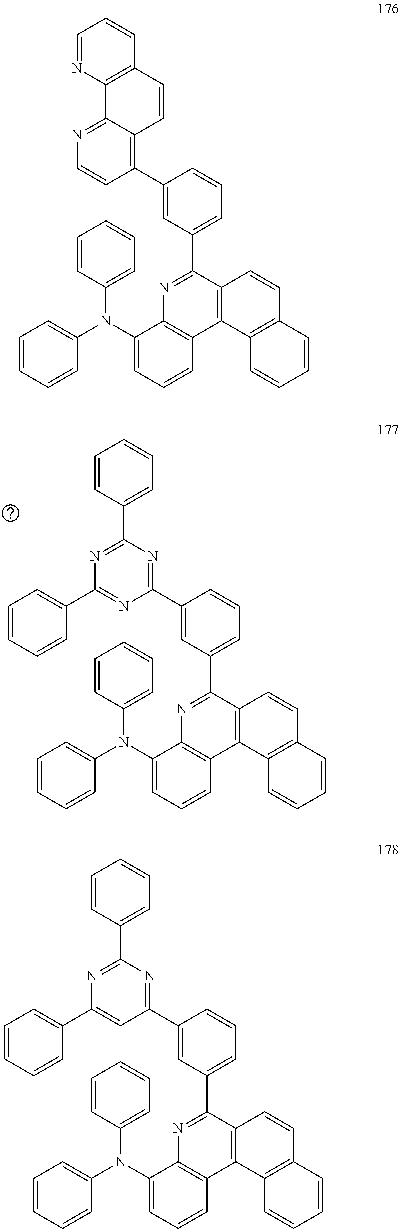 [SMILES: c1ccc(N(c2ccccc2)c2cccc3c2nc(-c2cccc(-c4ccnc5c4ccc4cccnc45)c2)c2ccc4ccccc4c23)cc1 ; c1ccc(-c2nc(-c3ccccc3)nc(-c3cccc(-c4nc5c(N(c6ccccc6)c6ccccc6)cccc5c5c4ccc4ccccc45)c3)n2)cc1 ; c1ccc(-c2cc(-c3cccc(-c4nc5c(N(c6ccccc6)c6ccccc6)cccc5c5c4ccc4ccccc45)c3)nc(-c3ccccc3)n2)cc1]

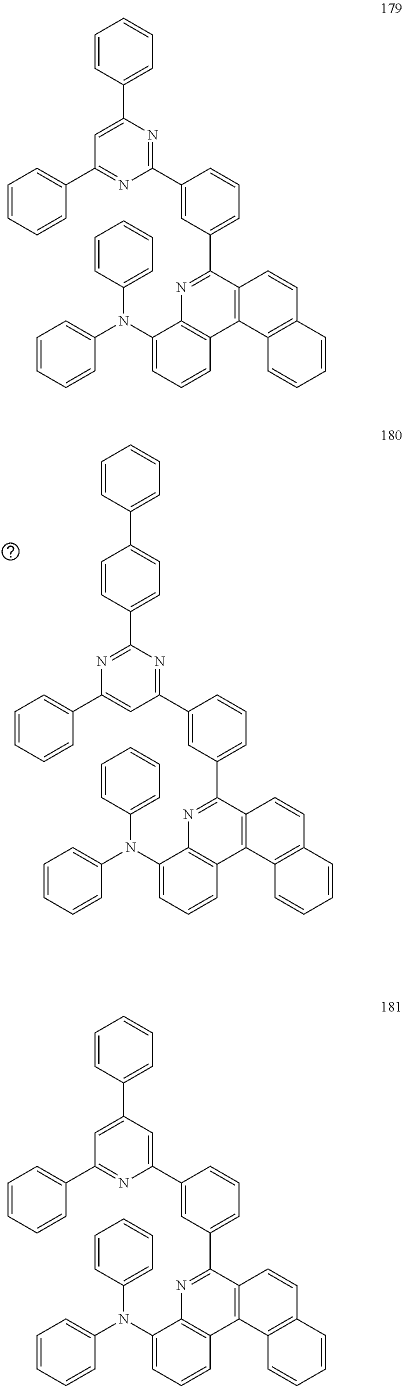 [SMILES: c1ccc(-c2cc(-c3ccccc3)nc(-c3cccc(-c4nc5c(N(c6ccccc6)c6ccccc6)cccc5c5c4ccc4ccccc45)c3)n2)cc1 ; c1ccc(-c2ccc(-c3nc(-c4ccccc4)cc(-c4cccc(-c5nc6c(N(c7ccccc7)c7ccccc7)cccc6c6c5ccc5ccccc56)c4)n3)cc2)cc1 ; c1ccc(-c2cc(-c3ccccc3)nc(-c3cccc(-c4nc5c(N(c6ccccc6)c6ccccc6)cccc5c5c4ccc4ccccc45)c3)c2)cc1]

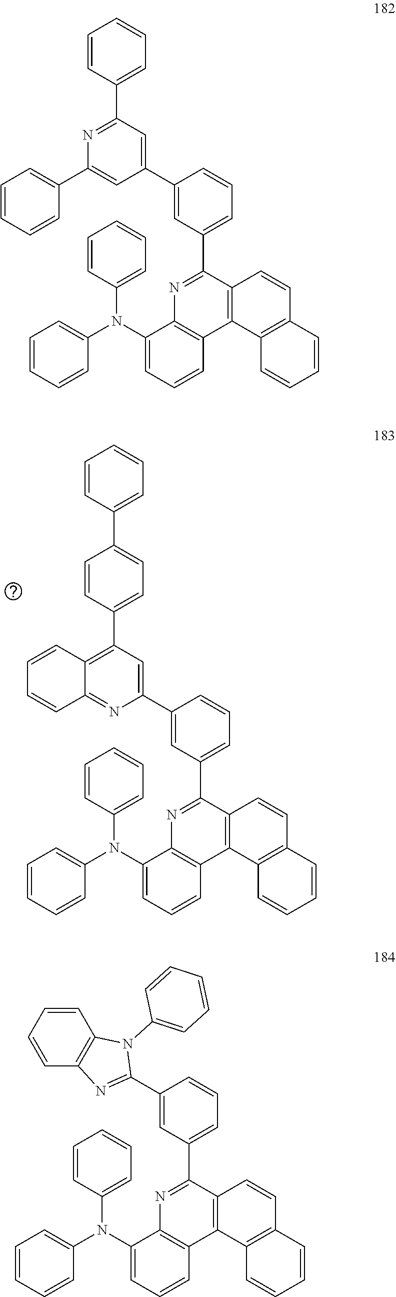 [SMILES: c1ccc(-c2cc(-c3cccc(-c4nc5c(N(c6ccccc6)c6ccccc6)cccc5c5c4ccc4ccccc45)c3)cc(-c3ccccc3)n2)cc1 ; c1ccc(-c2ccc(-c3cc(-c4cccc(-c5nc6c(N(c7ccccc7)c7ccccc7)cccc6c6c5ccc5ccccc56)c4)nc4ccccc34)cc2)cc1 ; c1ccc(N(c2ccccc2)c2cccc3c2nc(-c2cccc(-c4nc5ccccc5n4-c4ccccc4)c2)c2ccc4ccccc4c23)cc1]

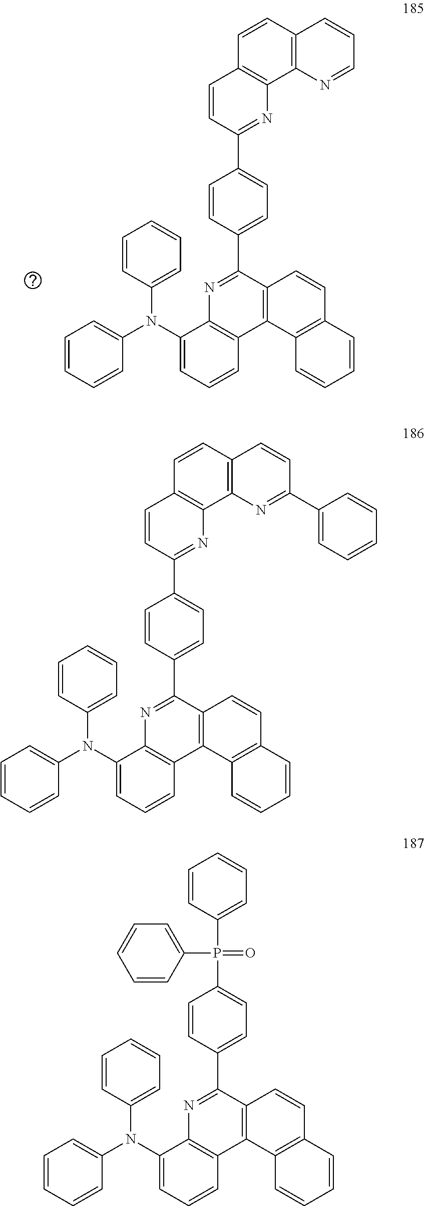 [SMILES: c1ccc(N(c2ccccc2)c2cccc3c2nc(-c2ccc(-c4ccc5ccc6cccnc6c5n4)cc2)c2ccc4ccccc4c23)cc1 ; c1ccc(-c2ccc3ccc4ccc(-c5ccc(-c6nc7c(N(c8ccccc8)c8ccccc8)cccc7c7c6ccc6ccccc67)cc5)nc4c3n2)cc1 ; O=P(c1ccccc1)(c1ccccc1)c1ccc(-c2nc3c(N(c4ccccc4)c4ccccc4)cccc3c3c2ccc2ccccc23)cc1]

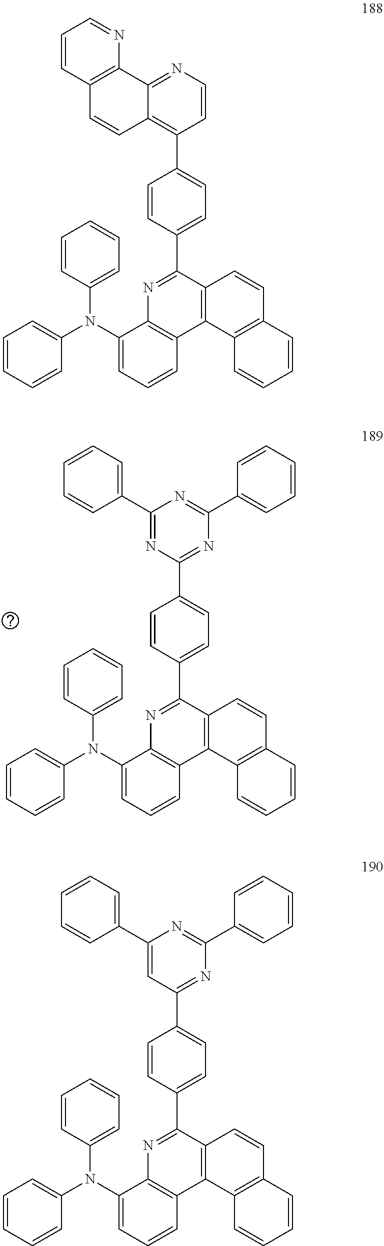 [SMILES: c1ccc(N(c2ccccc2)c2cccc3c2nc(-c2ccc(-c4ccnc5c4ccc4cccnc45)cc2)c2ccc4ccccc4c23)cc1 ; c1ccc(-c2nc(-c3ccccc3)nc(-c3ccc(-c4nc5c(N(c6ccccc6)c6ccccc6)cccc5c5c4ccc4ccccc45)cc3)n2)cc1 ; c1ccc(-c2cc(-c3ccc(-c4nc5c(N(c6ccccc6)c6ccccc6)cccc5c5c4ccc4ccccc45)cc3)nc(-c3ccccc3)n2)cc1]

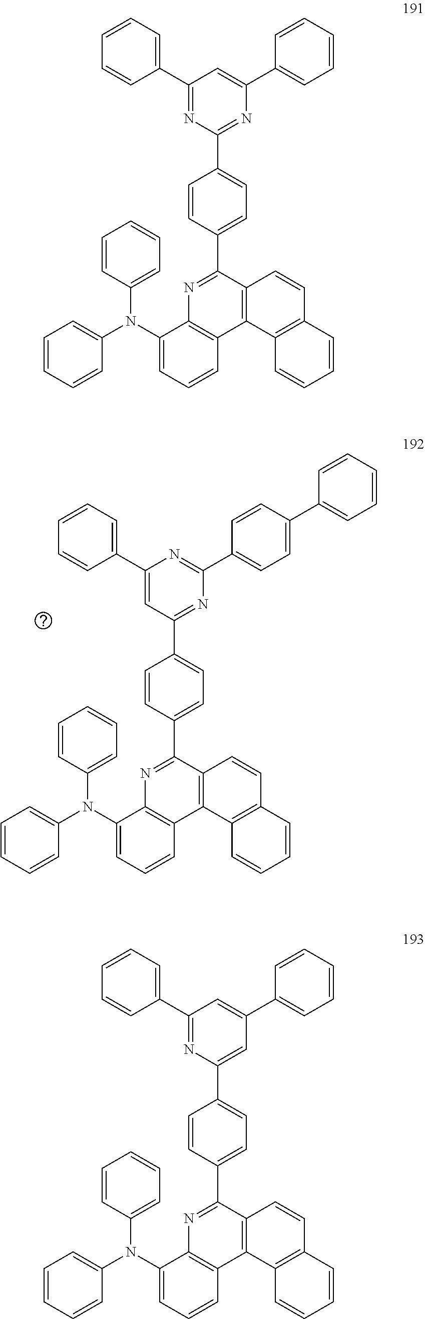 [SMILES: c1ccc(-c2cc(-c3ccccc3)nc(-c3ccc(-c4nc5c(N(c6ccccc6)c6ccccc6)cccc5c5c4ccc4ccccc45)cc3)n2)cc1 ; c1ccc(-c2ccc(-c3nc(-c4ccccc4)cc(-c4ccc(-c5nc6c(N(c7ccccc7)c7ccccc7)cccc6c6c5ccc5ccccc56)cc4)n3)cc2)cc1 ; c1ccc(-c2cc(-c3ccccc3)nc(-c3ccc(-c4nc5c(N(c6ccccc6)c6ccccc6)cccc5c5c4ccc4ccccc45)cc3)c2)cc1]

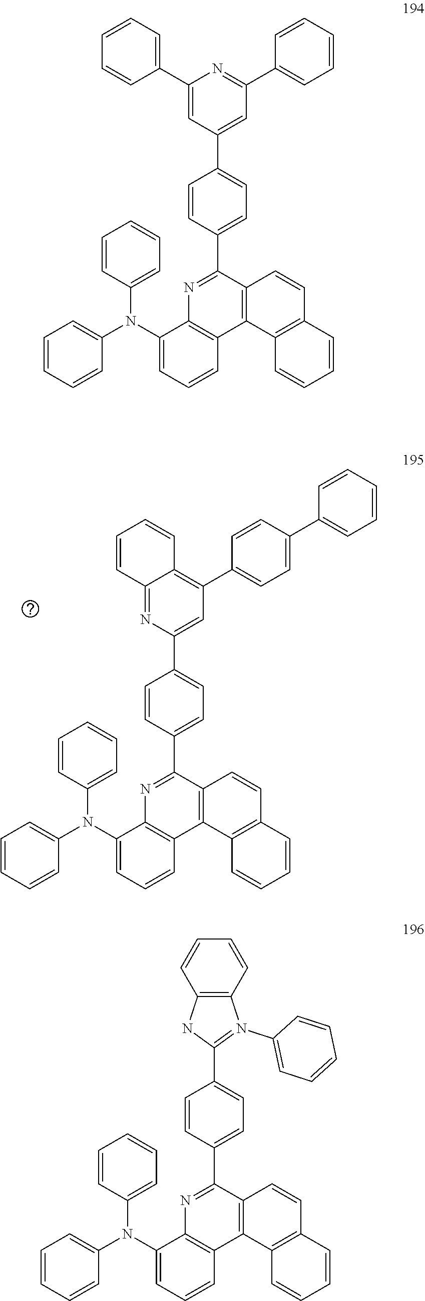

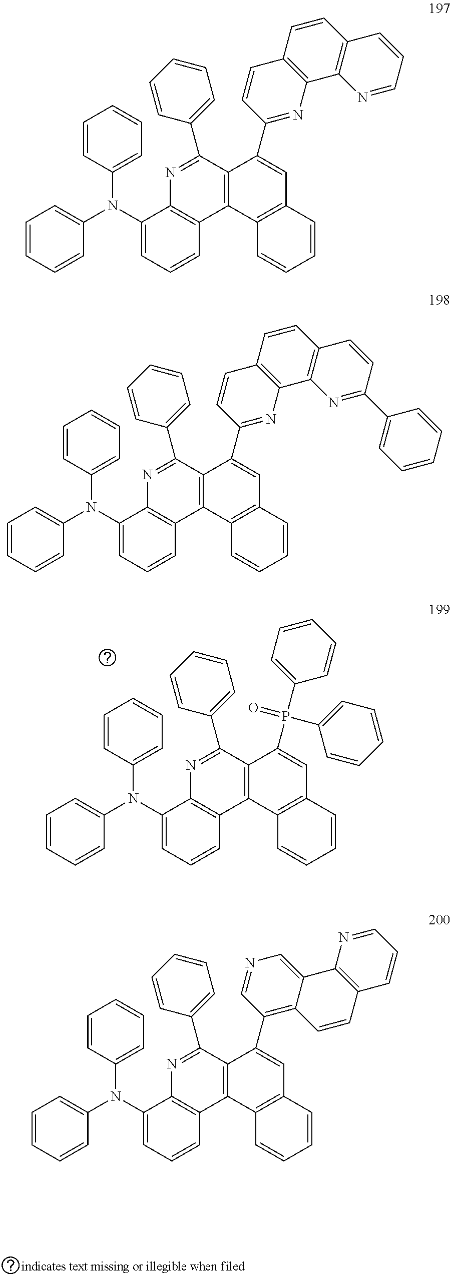 [SMILES: c1ccc(-c2nc3c(N(c4ccccc4)c4ccccc4)cccc3c3c2c(-c2ccc4ccc5cccnc5c4n2)cc2ccccc23)cc1 ; c1ccc(-c2ccc3ccc4ccc(-c5cc6ccccc6c6c5c(-c5ccccc5)nc5c(N(c7ccccc7)c7ccccc7)cccc56)nc4c3n2)cc1 ; O=P(c1ccccc1)(c1ccccc1)c1cc2ccccc2c2c1c(-c1ccccc1)nc1c(N(c3ccccc3)c3ccccc3)cccc12 ; c1ccc(-c2nc3c(N(c4ccccc4)c4ccccc4)cccc3c3c2c(-c2cncc4c2ccc2cccnc24)cc2ccccc23)cc1]

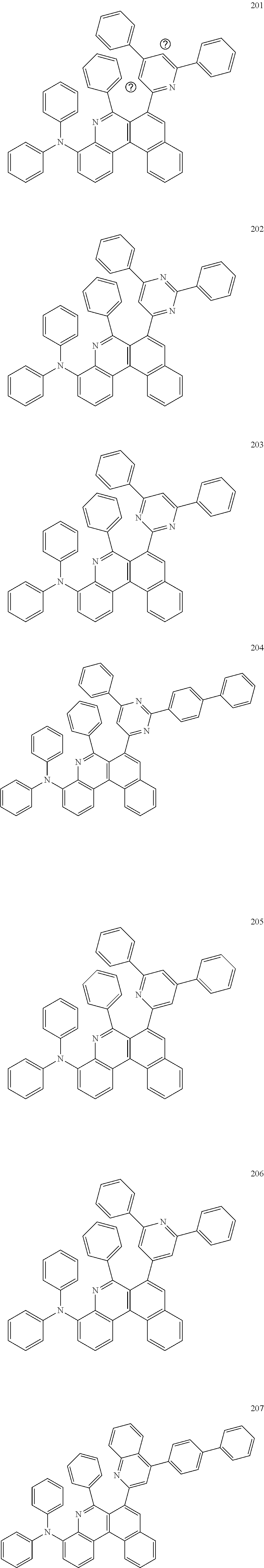

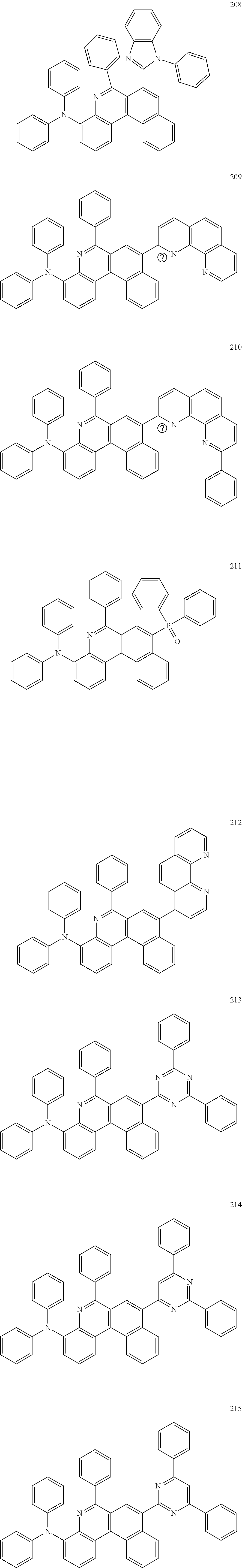

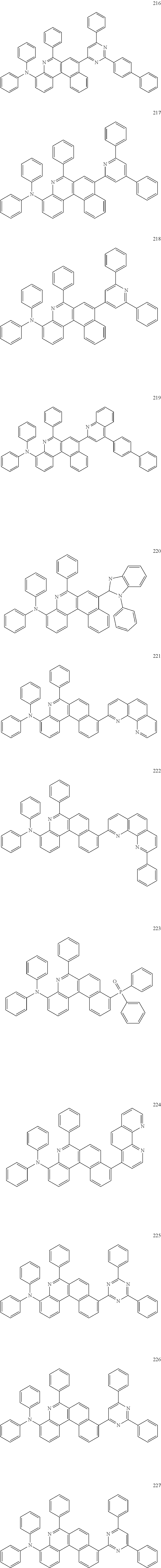

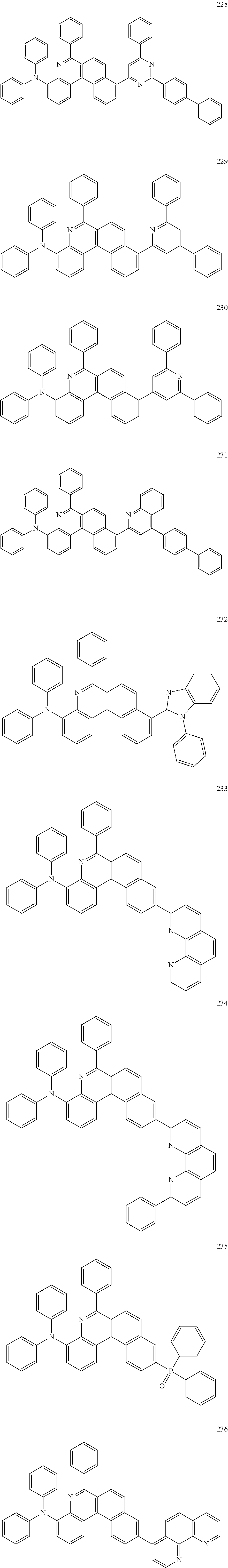

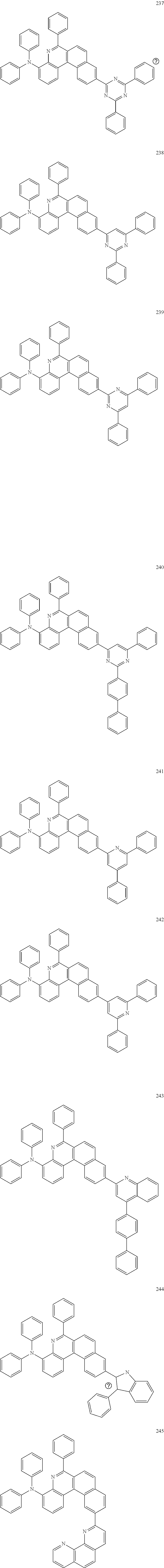

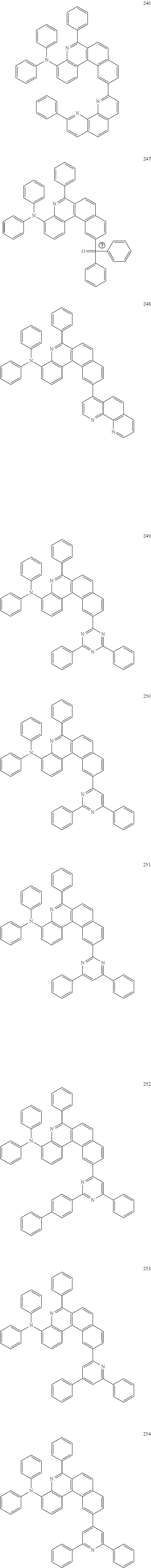

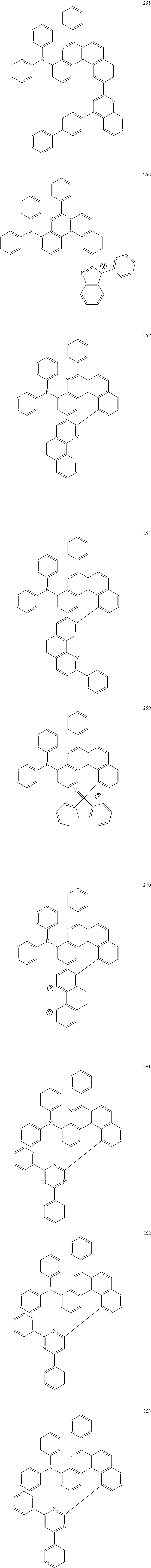

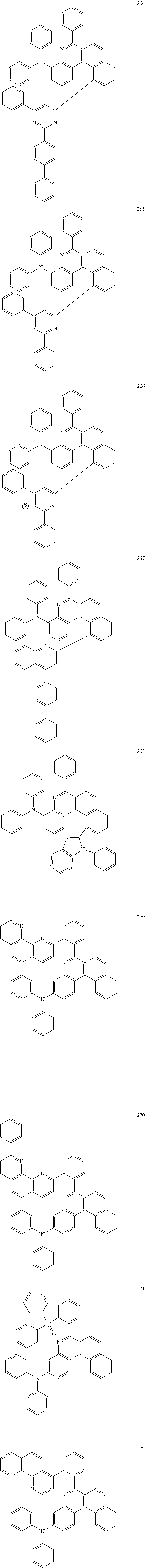

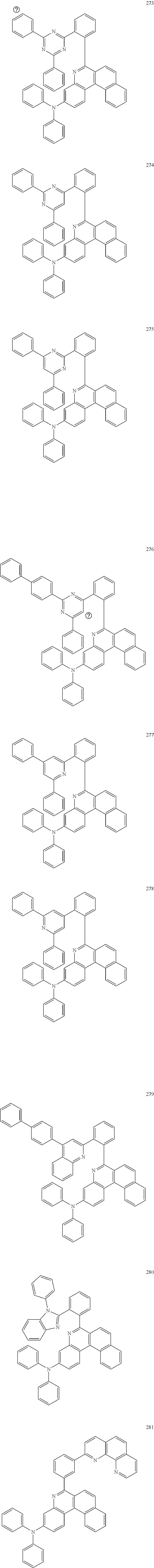

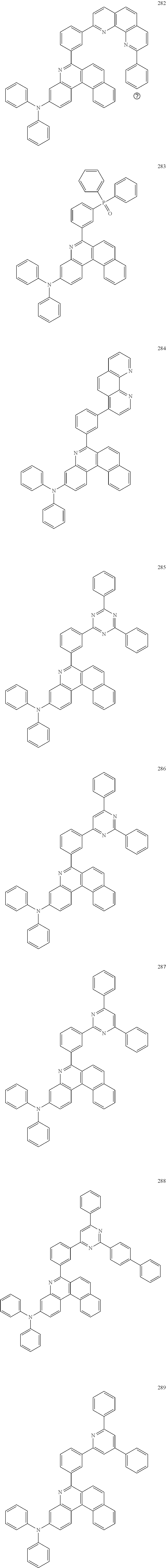

[chemical structure]

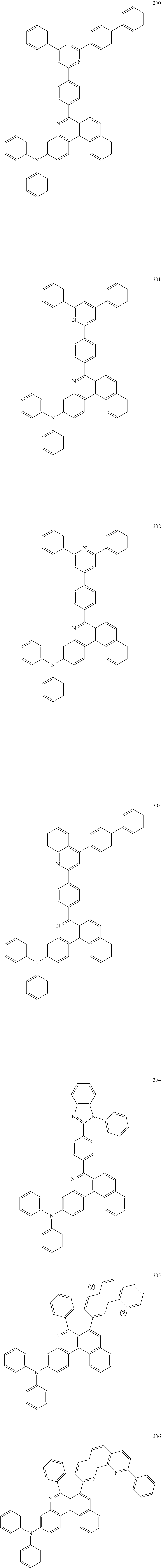

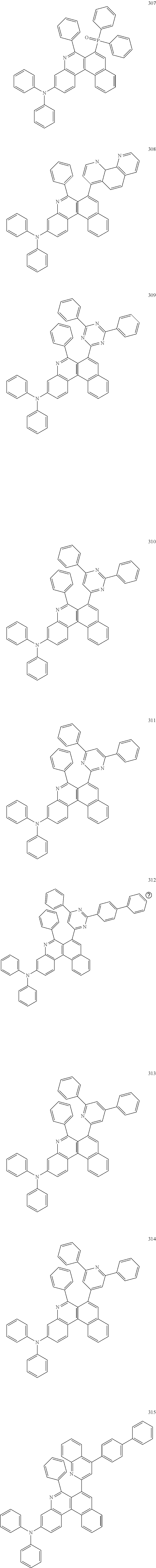

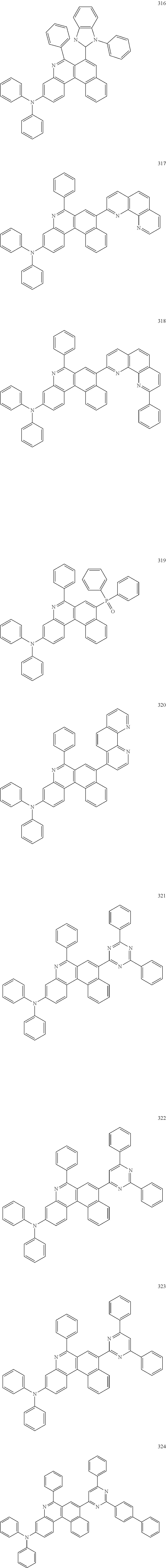

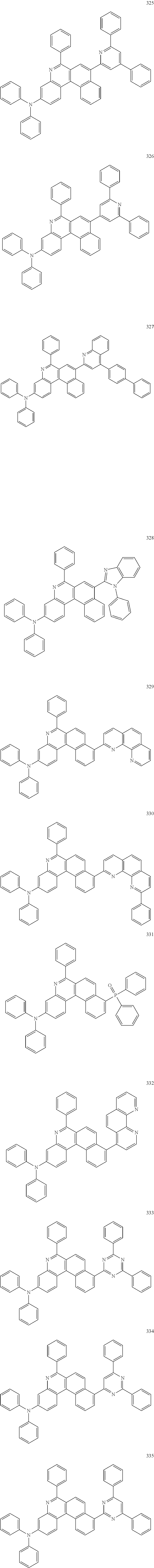

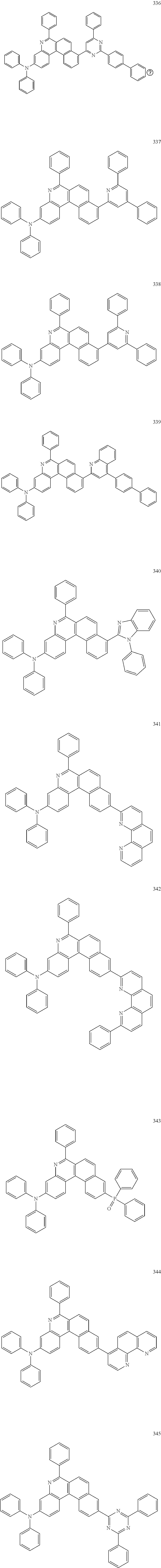

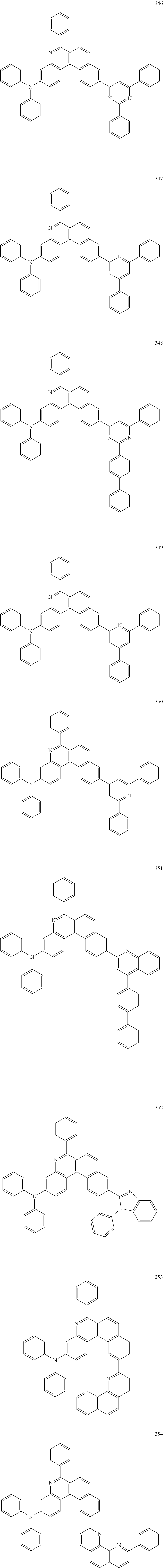

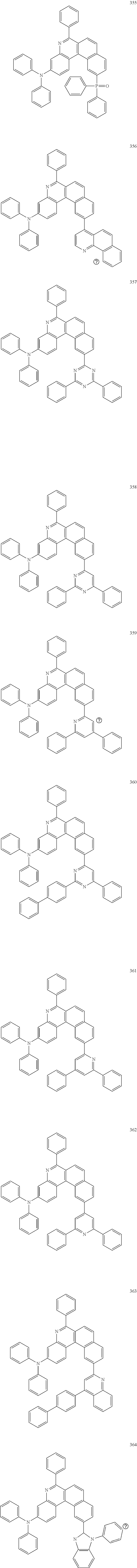

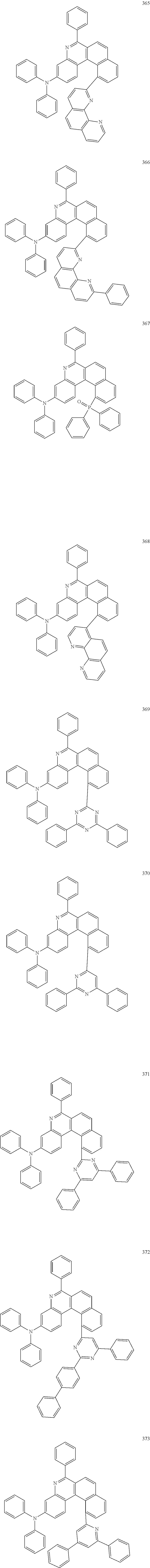

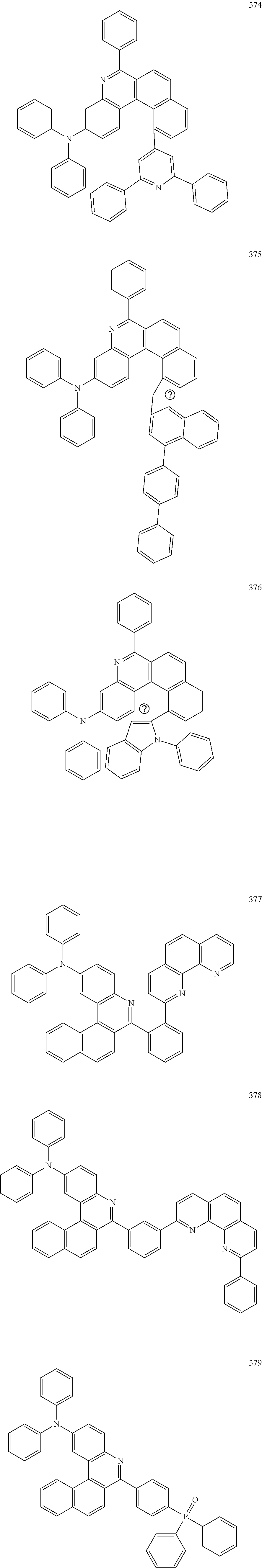

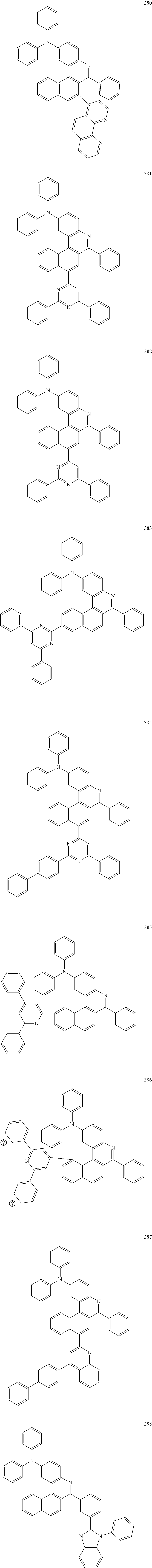

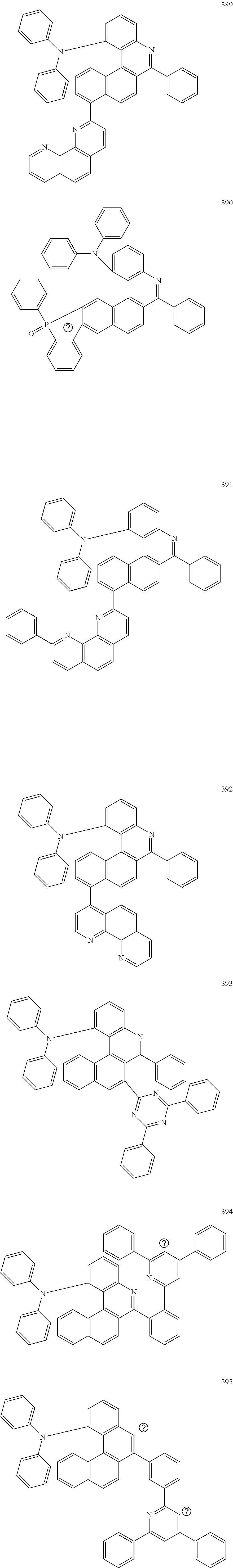

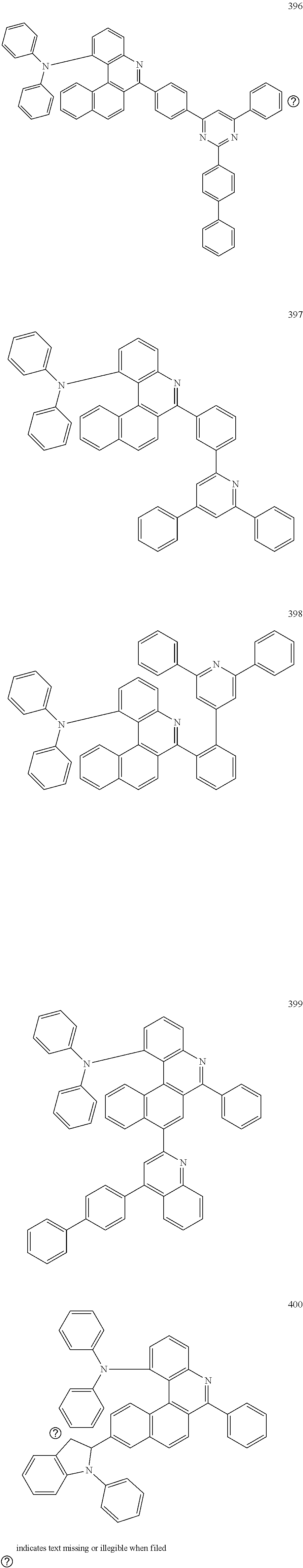

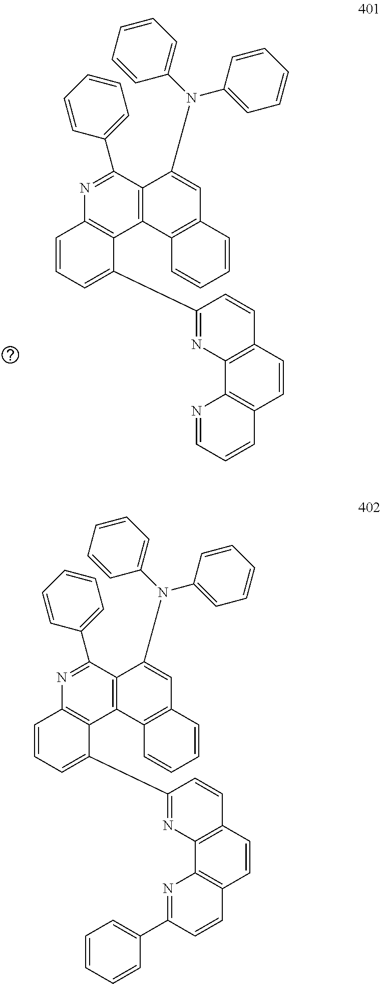 [SMILES: c1ccc(-c2nc3cccc(-c4ccc5ccc6cccnc6c5n4)c3c3c2c(N(c2ccccc2)c2ccccc2)cc2ccccc23)cc1 ; c1ccc(-c2ccc3ccc4ccc(-c5cccc6nc(-c7ccccc7)c7c(N(c8ccccc8)c8ccccc8)cc8ccccc8c7c56)nc4c3n2)cc1]

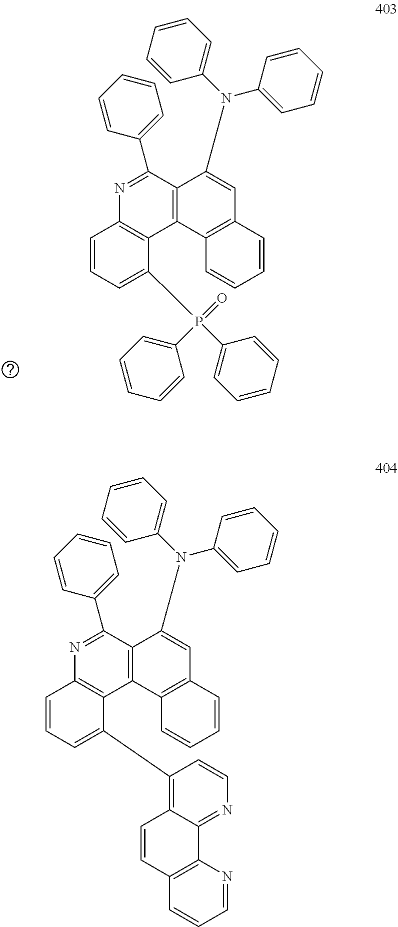 [SMILES: O=P(c1ccccc1)(c1ccccc1)c1cccc2nc(-c3ccccc3)c3c(N(c4ccccc4)c4ccccc4)cc4ccccc4c3c12 ; c1ccc(-c2nc3cccc(-c4ccnc5c4ccc4cccnc45)c3c3c2c(N(c2ccccc2)c2ccccc2)cc2ccccc23)cc1]

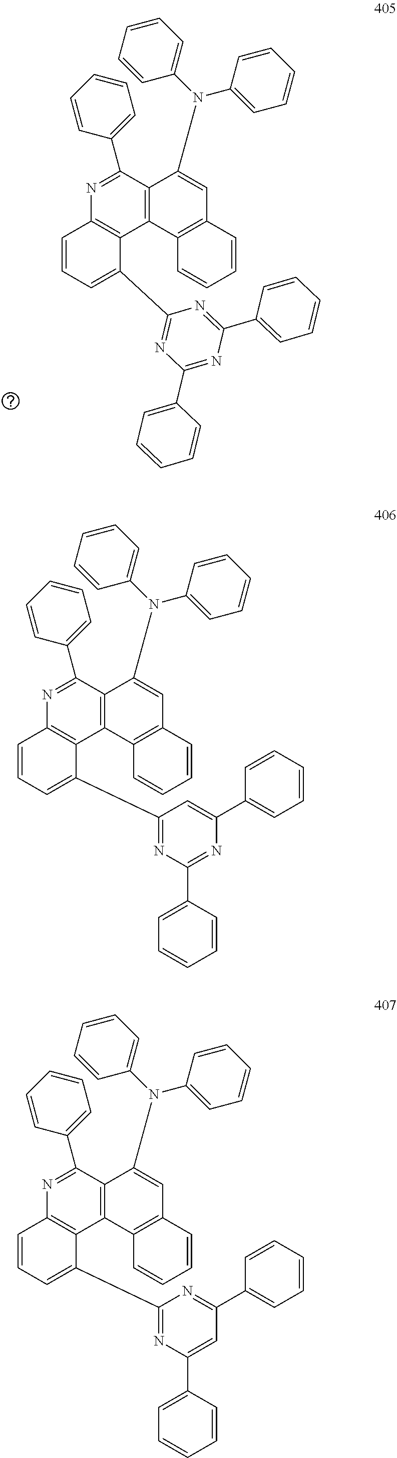 [SMILES: c1ccc(-c2nc(-c3ccccc3)nc(-c3cccc4nc(-c5ccccc5)c5c(N(c6ccccc6)c6ccccc6)cc6ccccc6c5c34)n2)cc1 ; c1ccc(-c2cc(-c3cccc4nc(-c5ccccc5)c5c(N(c6ccccc6)c6ccccc6)cc6ccccc6c5c34)nc(-c3ccccc3)n2)cc1 ; c1ccc(-c2cc(-c3ccccc3)nc(-c3cccc4nc(-c5ccccc5)c5c(N(c6ccccc6)c6ccccc6)cc6ccccc6c5c34)n2)cc1]

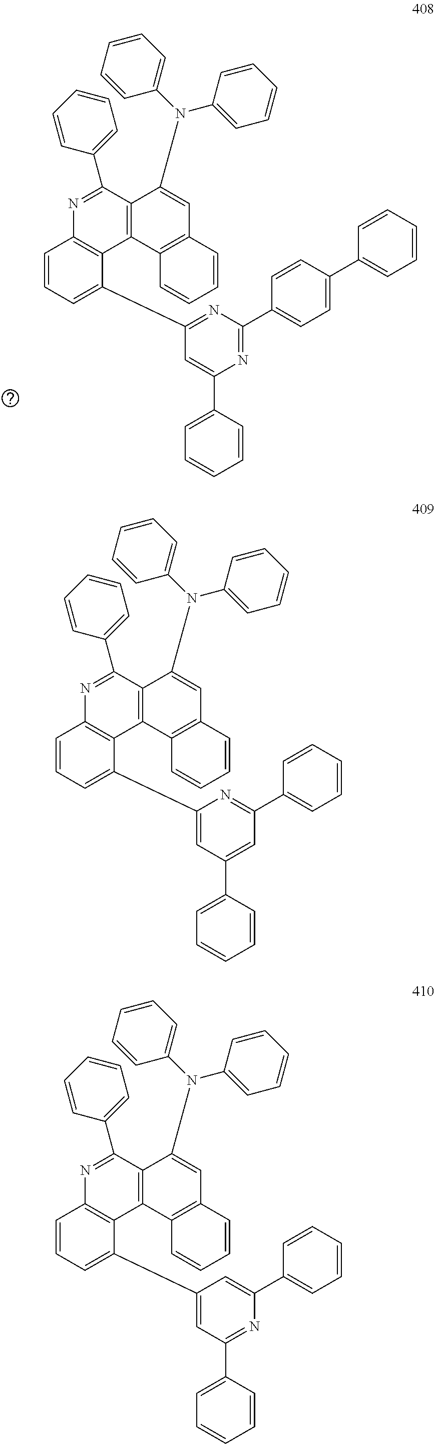 [SMILES: c1ccc(-c2ccc(-c3nc(-c4ccccc4)cc(-c4cccc5nc(-c6ccccc6)c6c(N(c7ccccc7)c7ccccc7)cc7ccccc7c6c45)n3)cc2)cc1 ; c1ccc(-c2cc(-c3ccccc3)nc(-c3cccc4nc(-c5ccccc5)c5c(N(c6ccccc6)c6ccccc6)cc6ccccc6c5c34)c2)cc1 ; c1ccc(-c2cc(-c3cccc4nc(-c5ccccc5)c5c(N(c6ccccc6)c6ccccc6)cc6ccccc6c5c34)cc(-c3ccccc3)n2)cc1]

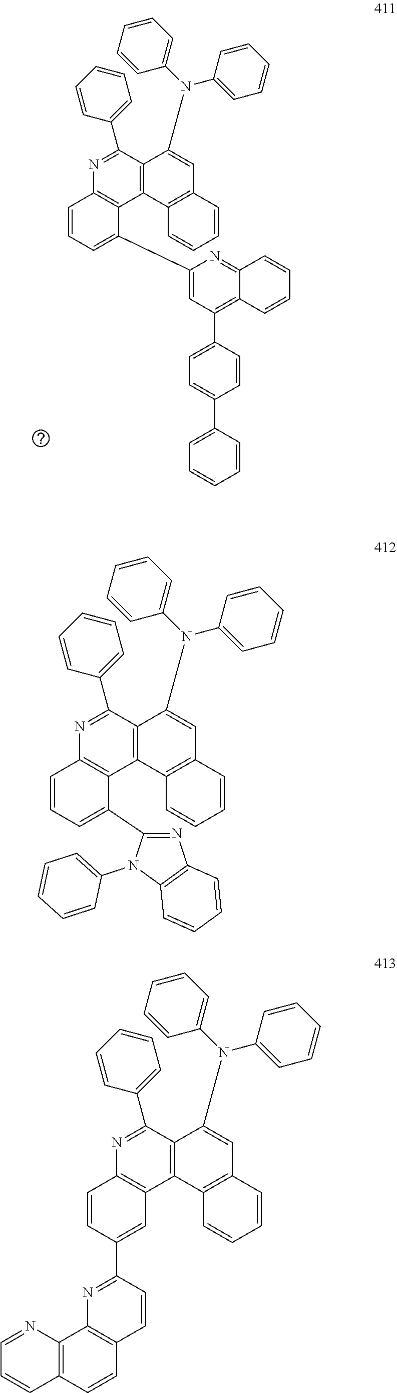

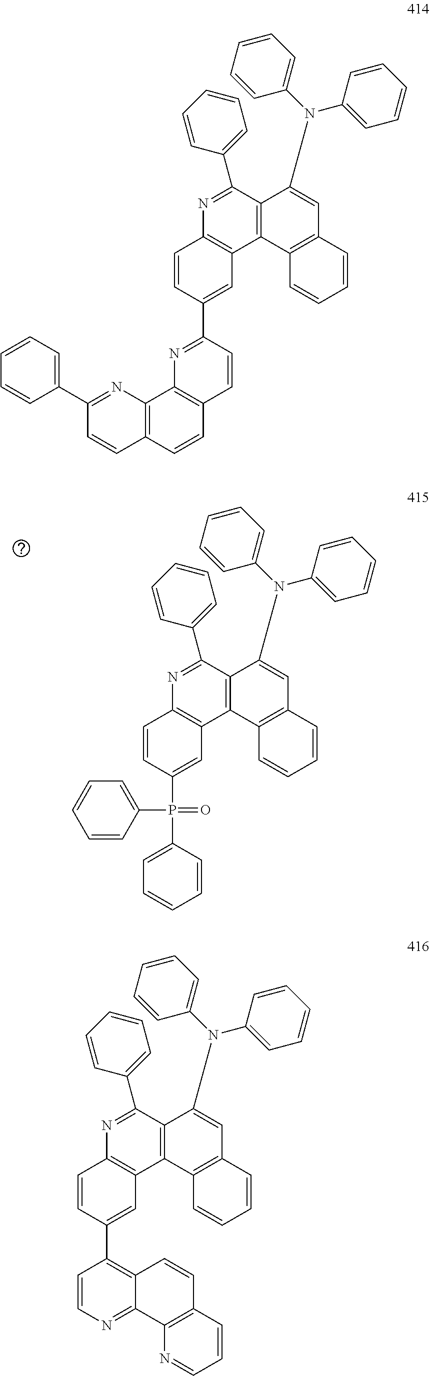

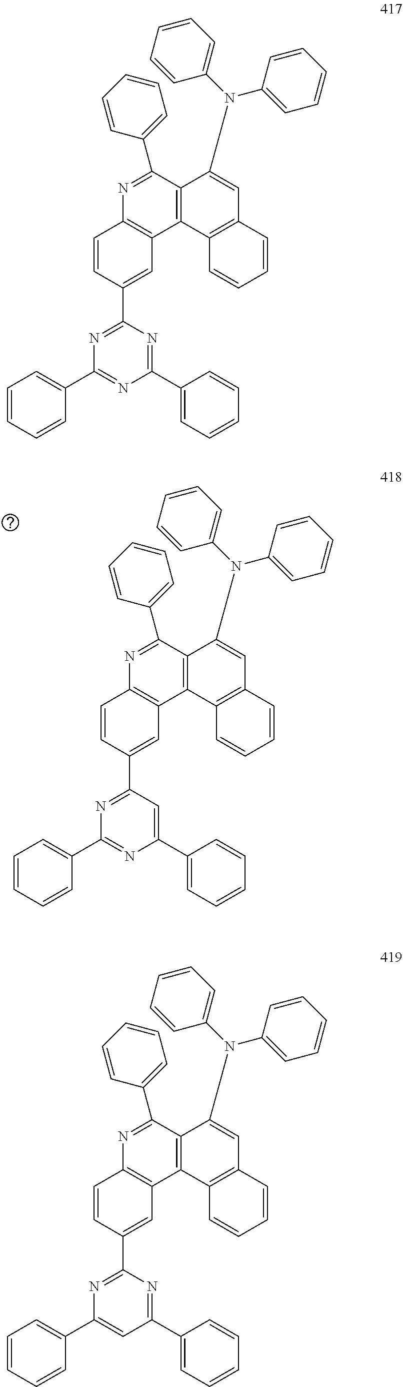 [SMILES: c1ccc(-c2nc(-c3ccccc3)nc(-c3ccc4nc(-c5ccccc5)c5c(N(c6ccccc6)c6ccccc6)cc6ccccc6c5c4c3)n2)cc1 ; c1ccc(-c2cc(-c3ccc4nc(-c5ccccc5)c5c(N(c6ccccc6)c6ccccc6)cc6ccccc6c5c4c3)nc(-c3ccccc3)n2)cc1 ; c1ccc(-c2cc(-c3ccccc3)nc(-c3ccc4nc(-c5ccccc5)c5c(N(c6ccccc6)c6ccccc6)cc6ccccc6c5c4c3)n2)cc1]

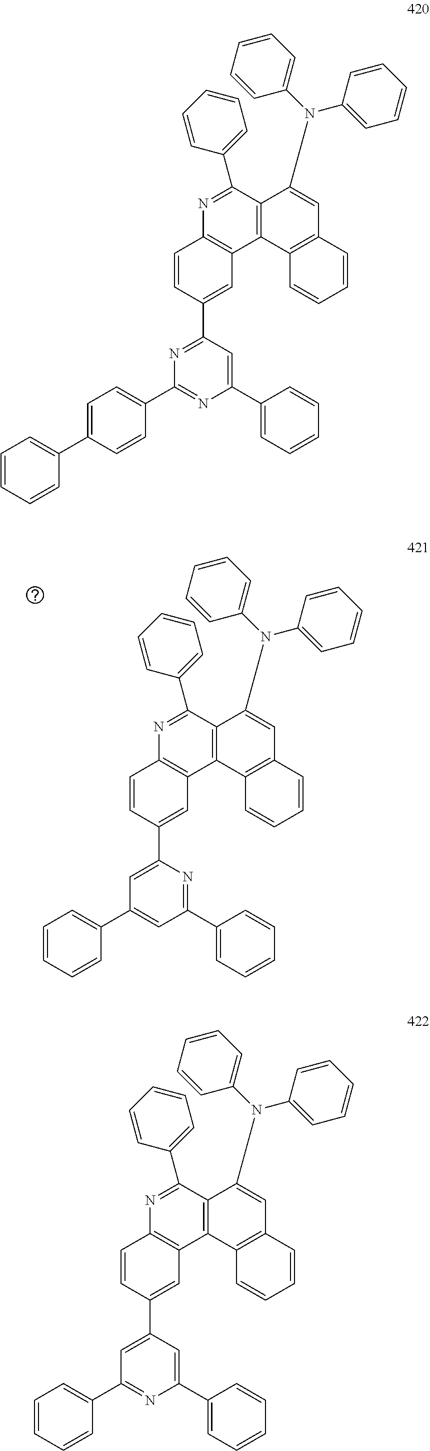 [SMILES: c1ccc(-c2ccc(-c3nc(-c4ccccc4)cc(-c4ccc5nc(-c6ccccc6)c6c(N(c7ccccc7)c7ccccc7)cc7ccccc7c6c5c4)n3)cc2)cc1 ; c1ccc(-c2cc(-c3ccccc3)nc(-c3ccc4nc(-c5ccccc5)c5c(N(c6ccccc6)c6ccccc6)cc6ccccc6c5c4c3)c2)cc1 ; c1ccc(-c2cc(-c3ccc4nc(-c5ccccc5)c5c(N(c6ccccc6)c6ccccc6)cc6ccccc6c5c4c3)cc(-c3ccccc3)n2)cc1]

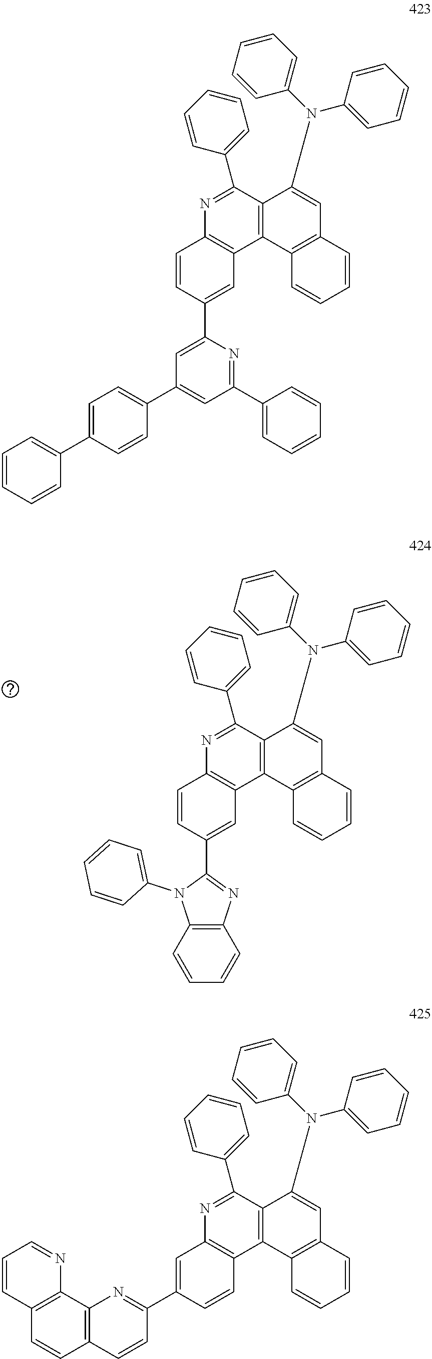 [SMILES: c1ccc(-c2ccc(-c3cc(-c4ccccc4)nc(-c4ccc5nc(-c6ccccc6)c6c(N(c7ccccc7)c7ccccc7)cc7ccccc7c6c5c4)c3)cc2)cc1 ; c1ccc(-c2nc3ccc(-c4nc5ccccc5n4-c4ccccc4)cc3c3c2c(N(c2ccccc2)c2ccccc2)cc2ccccc23)cc1 ; c1ccc(-c2nc3cc(-c4ccc5ccc6cccnc6c5n4)ccc3c3c2c(N(c2ccccc2)c2ccccc2)cc2ccccc23)cc1]

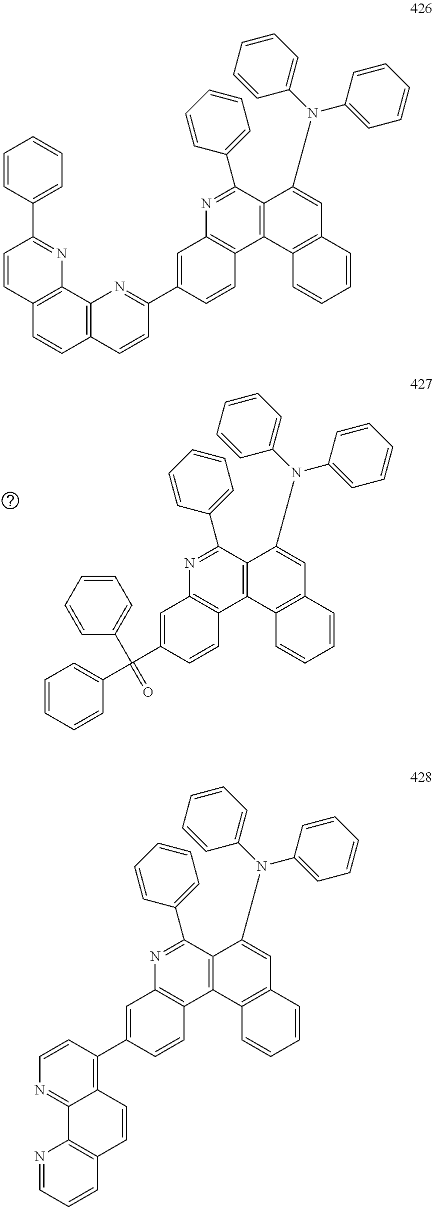

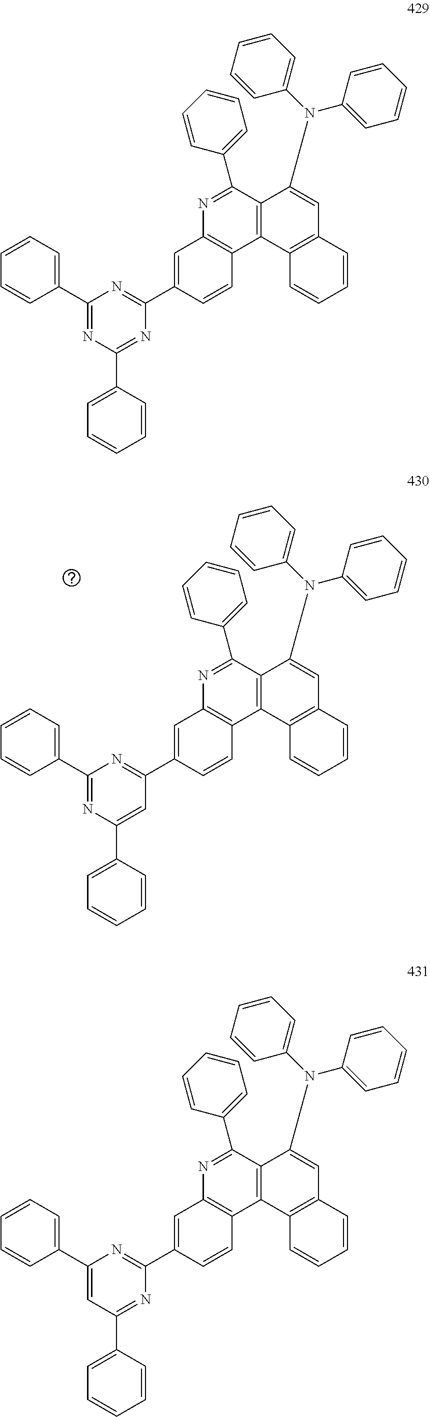 [SMILES: c1ccc(-c2nc(-c3ccccc3)nc(-c3ccc4c(c3)nc(-c3ccccc3)c3c(N(c5ccccc5)c5ccccc5)cc5ccccc5c34)n2)cc1 ; c1ccc(-c2cc(-c3ccc4c(c3)nc(-c3ccccc3)c3c(N(c5ccccc5)c5ccccc5)cc5ccccc5c34)nc(-c3ccccc3)n2)cc1 ; c1ccc(-c2cc(-c3ccccc3)nc(-c3ccc4c(c3)nc(-c3ccccc3)c3c(N(c5ccccc5)c5ccccc5)cc5ccccc5c34)n2)cc1]

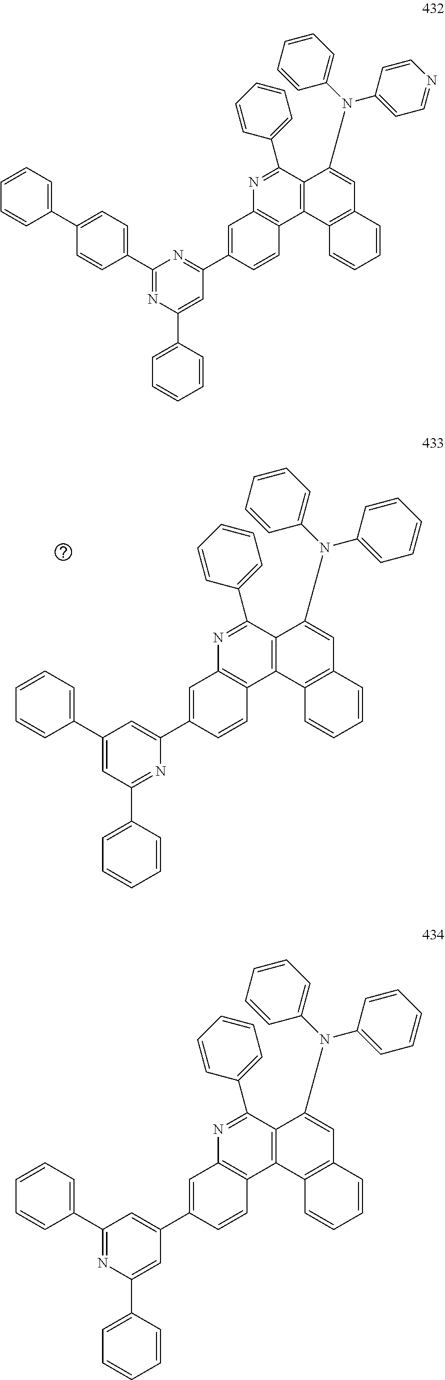 [SMILES: c1ccc(-c2ccc(-c3nc(-c4ccccc4)cc(-c4ccc5c(c4)nc(-c4ccccc4)c4c(N(c6ccccc6)c6ccncc6)cc6ccccc6c45)n3)cc2)cc1 ; c1ccc(-c2cc(-c3ccccc3)nc(-c3ccc4c(c3)nc(-c3ccccc3)c3c(N(c5ccccc5)c5ccccc5)cc5ccccc5c34)c2)cc1 ; c1ccc(-c2cc(-c3ccc4c(c3)nc(-c3ccccc3)c3c(N(c5ccccc5)c5ccccc5)cc5ccccc5c34)cc(-c3ccccc3)n2)cc1]

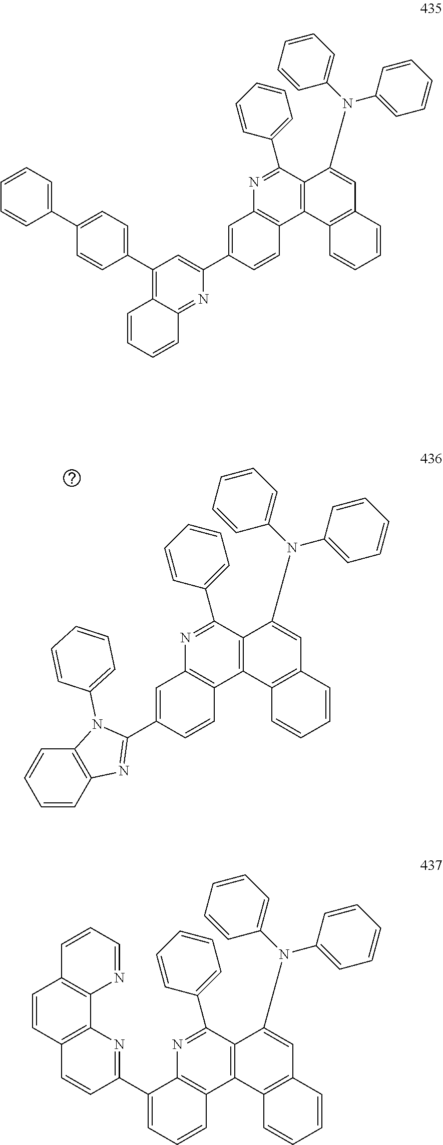 [SMILES: c1ccc(-c2ccc(-c3cc(-c4ccc5c(c4)nc(-c4ccccc4)c4c(N(c6ccccc6)c6ccccc6)cc6ccccc6c45)nc4ccccc34)cc2)cc1 ; c1ccc(-c2nc3cc(-c4nc5ccccc5n4-c4ccccc4)ccc3c3c2c(N(c2ccccc2)c2ccccc2)cc2ccccc23)cc1 ; c1ccc(-c2nc3c(-c4ccc5ccc6cccnc6c5n4)cccc3c3c2c(N(c2ccccc2)c2ccccc2)cc2ccccc23)cc1]

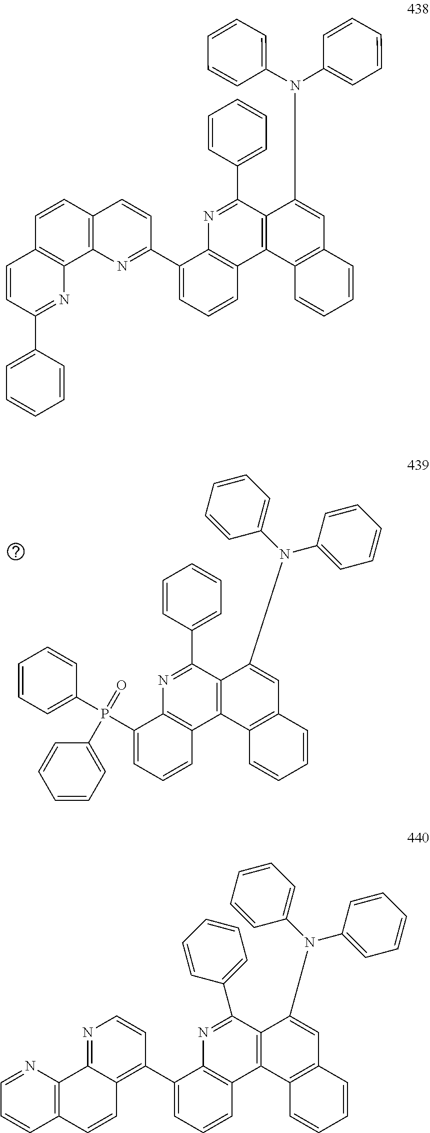 [SMILES: c1ccc(-c2ccc3ccc4ccc(-c5cccc6c5nc(-c5ccccc5)c5c(N(c7ccccc7)c7ccccc7)cc7ccccc7c56)nc4c3n2)cc1 ; O=P(c1ccccc1)(c1ccccc1)c1cccc2c1nc(-c1ccccc1)c1c(N(c3ccccc3)c3ccccc3)cc3ccccc3c12 ; c1ccc(-c2nc3c(-c4ccnc5c4ccc4cccnc45)cccc3c3c2c(N(c2ccccc2)c2ccccc2)cc2ccccc23)cc1]

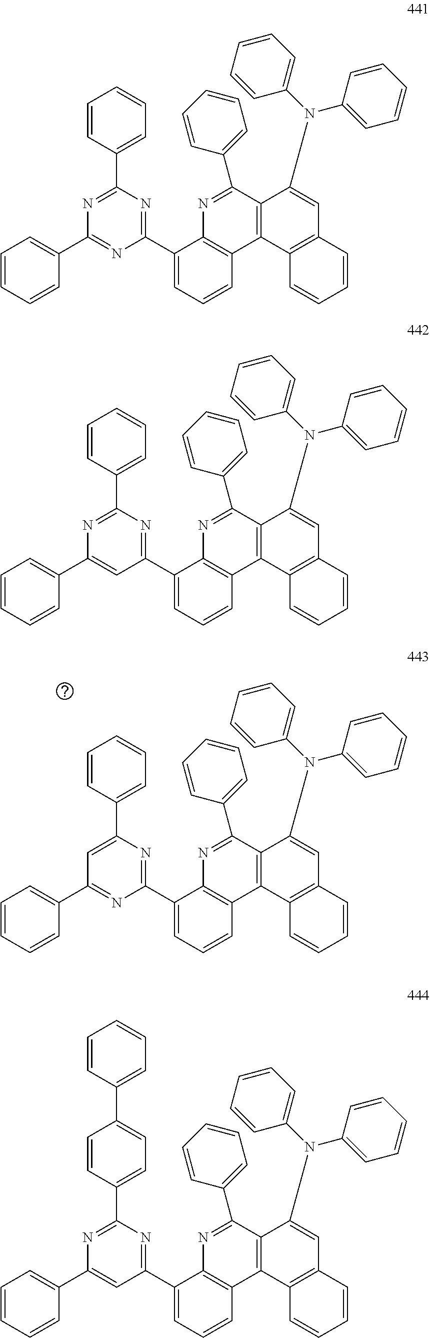 [SMILES: c1ccc(-c2nc(-c3ccccc3)nc(-c3cccc4c3nc(-c3ccccc3)c3c(N(c5ccccc5)c5ccccc5)cc5ccccc5c34)n2)cc1 ; c1ccc(-c2cc(-c3cccc4c3nc(-c3ccccc3)c3c(N(c5ccccc5)c5ccccc5)cc5ccccc5c34)nc(-c3ccccc3)n2)cc1 ; c1ccc(-c2cc(-c3ccccc3)nc(-c3cccc4c3nc(-c3ccccc3)c3c(N(c5ccccc5)c5ccccc5)cc5ccccc5c34)n2)cc1 ; c1ccc(-c2ccc(-c3nc(-c4ccccc4)cc(-c4cccc5c4nc(-c4ccccc4)c4c(N(c6ccccc6)c6ccccc6)cc6ccccc6c45)n3)cc2)cc1]

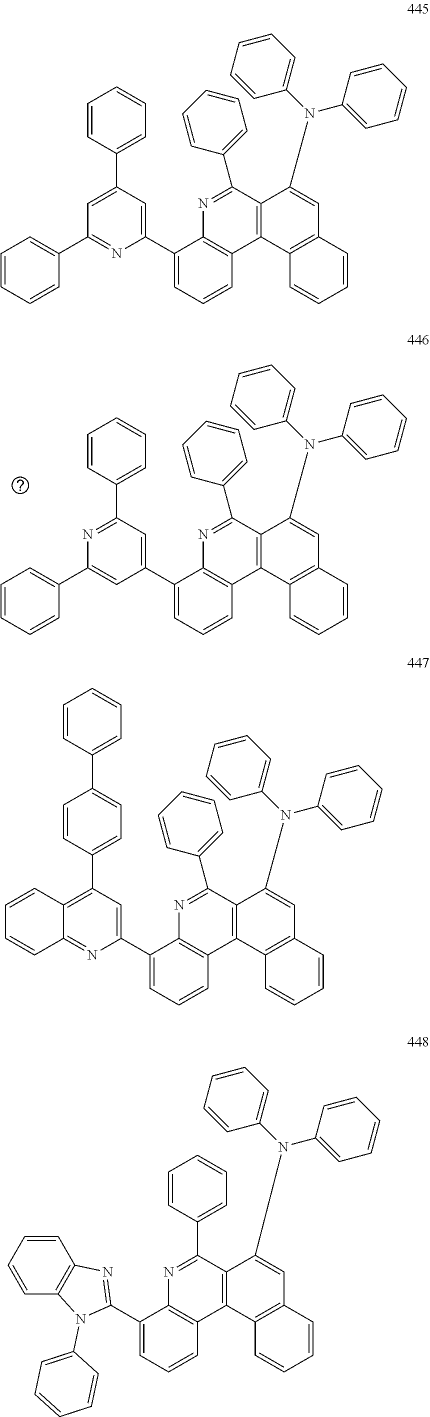 [SMILES: c1ccc(-c2cc(-c3ccccc3)nc(-c3cccc4c3nc(-c3ccccc3)c3c(N(c5ccccc5)c5ccccc5)cc5ccccc5c34)c2)cc1 ; c1ccc(-c2cc(-c3cccc4c3nc(-c3ccccc3)c3c(N(c5ccccc5)c5ccccc5)cc5ccccc5c34)cc(-c3ccccc3)n2)cc1 ; c1ccc(-c2ccc(-c3cc(-c4cccc5c4nc(-c4ccccc4)c4c(N(c6ccccc6)c6ccccc6)cc6ccccc6c45)nc4ccccc34)cc2)cc1 ; c1ccc(-c2nc3c(-c4nc5ccccc5n4-c4ccccc4)cccc3c3c2c(N(c2ccccc2)c2ccccc2)cc2ccccc23)cc1]

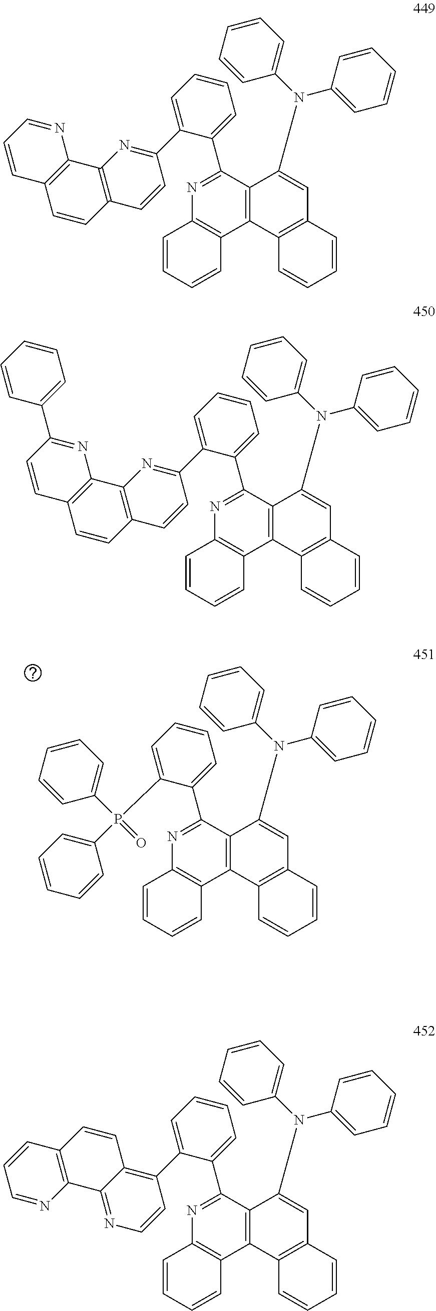 [SMILES: c1ccc(N(c2ccccc2)c2cc3ccccc3c3c2c(-c2ccccc2-c2ccc4ccc5cccnc5c4n2)nc2ccccc23)cc1 ; c1ccc(-c2ccc3ccc4ccc(-c5ccccc5-c5nc6ccccc6c6c5c(N(c5ccccc5)c5ccccc5)cc5ccccc56)nc4c3n2)cc1 ; O=P(c1ccccc1)(c1ccccc1)c1ccccc1-c1nc2ccccc2c2c1c(N(c1ccccc1)c1ccccc1)cc1ccccc12 ; c1ccc(N(c2ccccc2)c2cc3ccccc3c3c2c(-c2ccccc2-c2ccnc4c2ccc2cccnc24)nc2ccccc23)cc1]

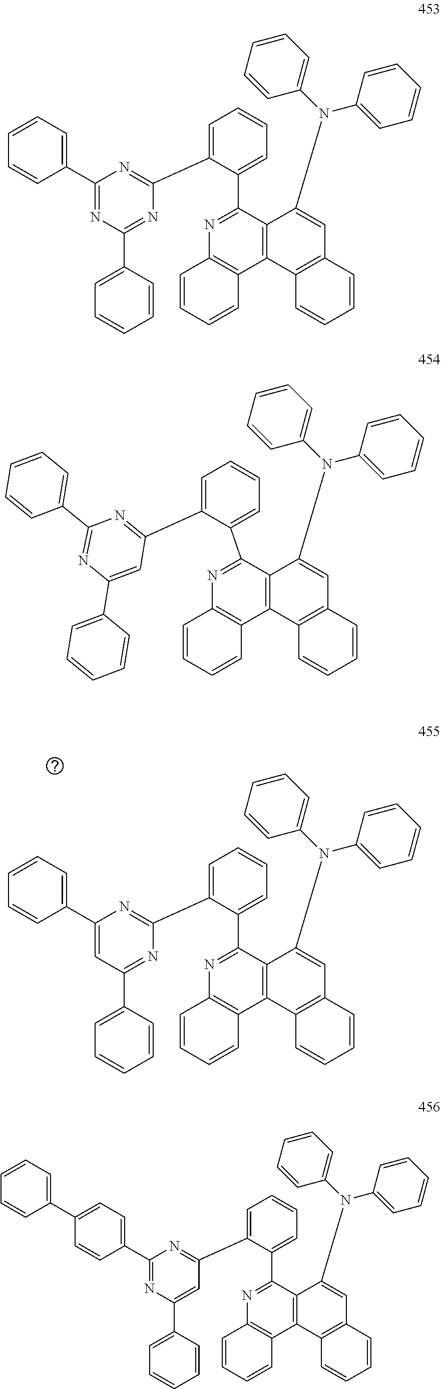 [SMILES: c1ccc(-c2nc(-c3ccccc3)nc(-c3ccccc3-c3nc4ccccc4c4c3c(N(c3ccccc3)c3ccccc3)cc3ccccc34)n2)cc1 ; c1ccc(-c2cc(-c3ccccc3-c3nc4ccccc4c4c3c(N(c3ccccc3)c3ccccc3)cc3ccccc34)nc(-c3ccccc3)n2)cc1 ; c1ccc(-c2cc(-c3ccccc3)nc(-c3ccccc3-c3nc4ccccc4c4c3c(N(c3ccccc3)c3ccccc3)cc3ccccc34)n2)cc1 ; c1ccc(-c2ccc(-c3nc(-c4ccccc4)cc(-c4ccccc4-c4nc5ccccc5c5c4c(N(c4ccccc4)c4ccccc4)cc4ccccc45)n3)cc2)cc1]

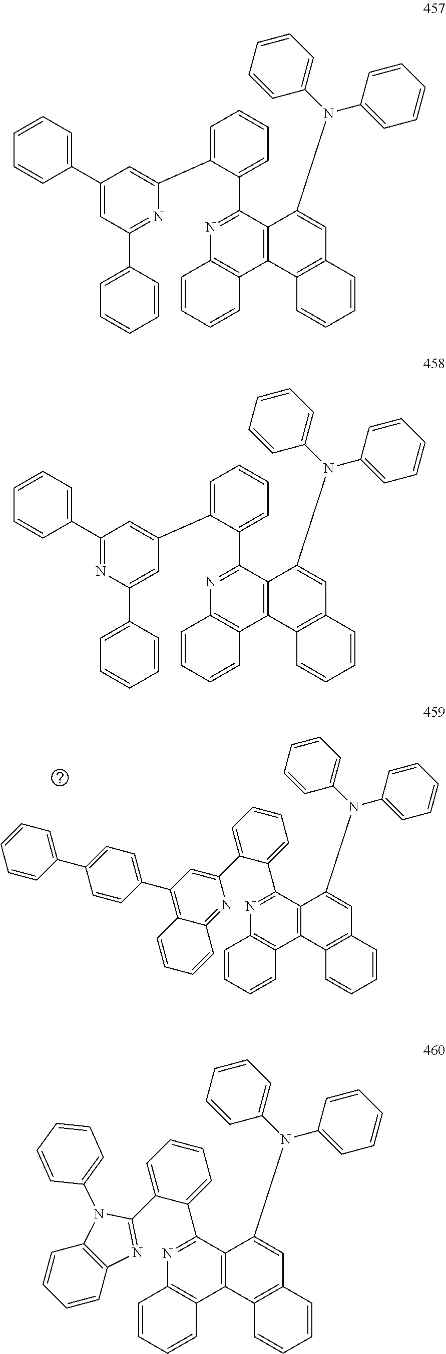 [SMILES: c1ccc(-c2cc(-c3ccccc3)nc(-c3ccccc3-c3nc4ccccc4c4c3c(N(c3ccccc3)c3ccccc3)cc3ccccc34)c2)cc1 ; c1ccc(-c2cc(-c3ccccc3-c3nc4ccccc4c4c3c(N(c3ccccc3)c3ccccc3)cc3ccccc34)cc(-c3ccccc3)n2)cc1 ; c1ccc(-c2ccc(-c3cc(-c4ccccc4-c4nc5ccccc5c5c4c(N(c4ccccc4)c4ccccc4)cc4ccccc45)nc4ccccc34)cc2)cc1 ; c1ccc(N(c2ccccc2)c2cc3ccccc3c3c2c(-c2ccccc2-c2nc4ccccc4n2-c2ccccc2)nc2ccccc23)cc1]

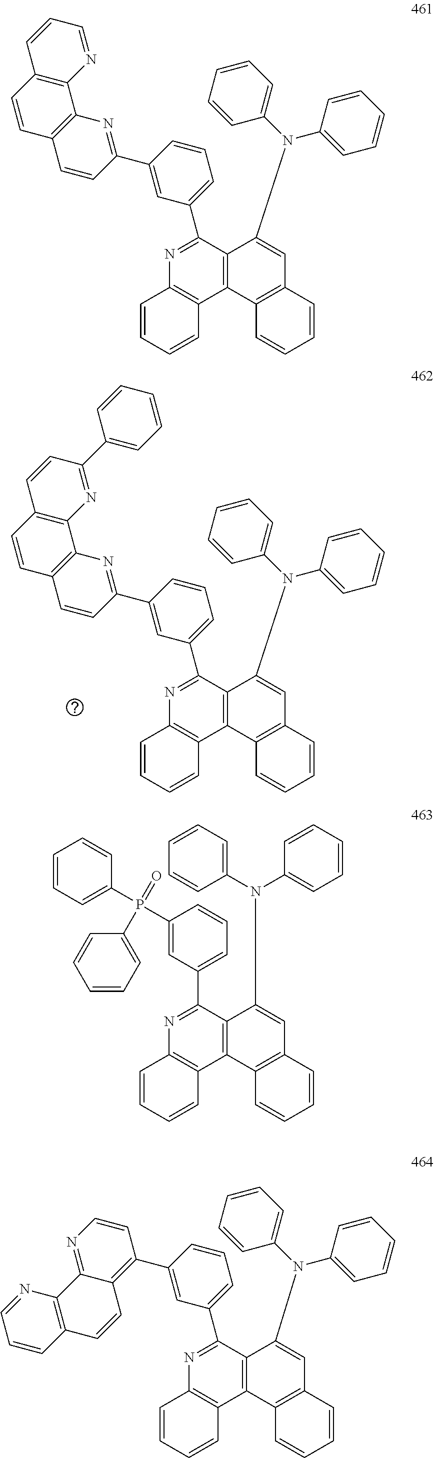

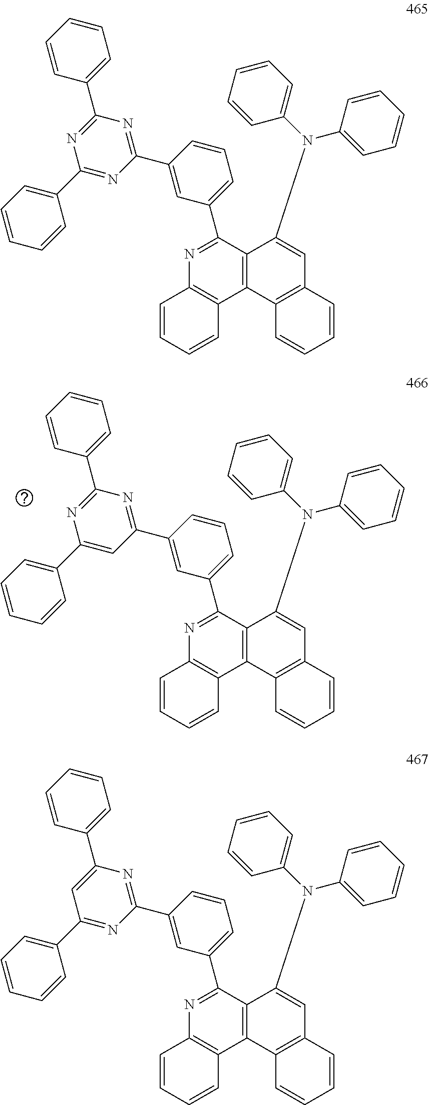 [SMILES: c1ccc(-c2nc(-c3ccccc3)nc(-c3cccc(-c4nc5ccccc5c5c4c(N(c4ccccc4)c4ccccc4)cc4ccccc45)c3)n2)cc1 ; c1ccc(-c2cc(-c3cccc(-c4nc5ccccc5c5c4c(N(c4ccccc4)c4ccccc4)cc4ccccc45)c3)nc(-c3ccccc3)n2)cc1 ; c1ccc(-c2cc(-c3ccccc3)nc(-c3cccc(-c4nc5ccccc5c5c4c(N(c4ccccc4)c4ccccc4)cc4ccccc45)c3)n2)cc1]

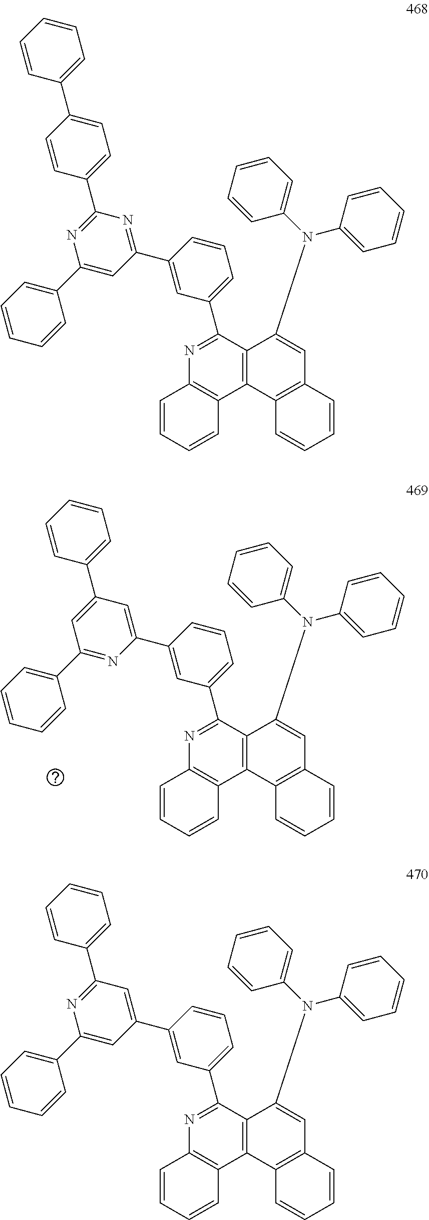 [SMILES: c1ccc(-c2ccc(-c3nc(-c4ccccc4)cc(-c4cccc(-c5nc6ccccc6c6c5c(N(c5ccccc5)c5ccccc5)cc5ccccc56)c4)n3)cc2)cc1 ; c1ccc(-c2cc(-c3ccccc3)nc(-c3cccc(-c4nc5ccccc5c5c4c(N(c4ccccc4)c4ccccc4)cc4ccccc45)c3)c2)cc1 ; c1ccc(-c2cc(-c3cccc(-c4nc5ccccc5c5c4c(N(c4ccccc4)c4ccccc4)cc4ccccc45)c3)cc(-c3ccccc3)n2)cc1]

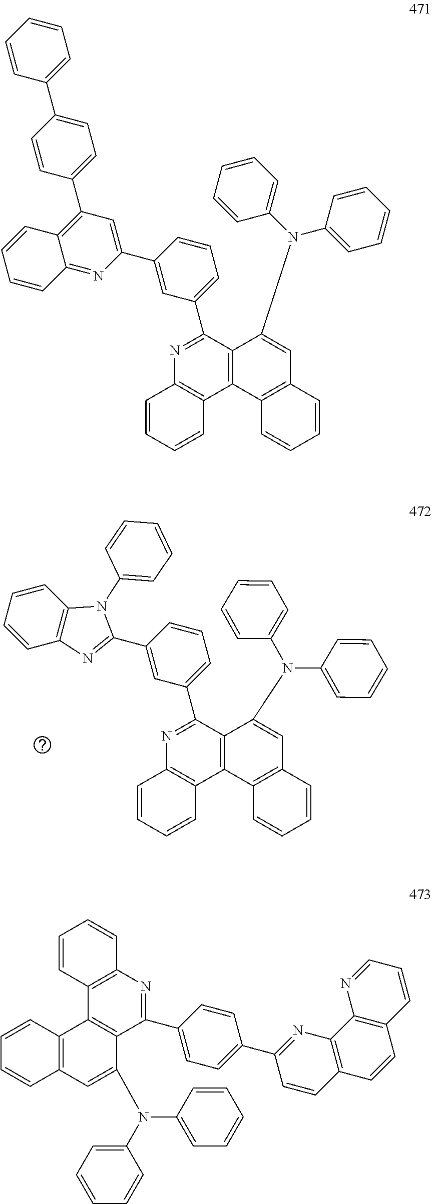 [SMILES: c1ccc(-c2ccc(-c3cc(-c4cccc(-c5nc6ccccc6c6c5c(N(c5ccccc5)c5ccccc5)cc5ccccc56)c4)nc4ccccc34)cc2)cc1 ; c1ccc(N(c2ccccc2)c2cc3ccccc3c3c2c(-c2cccc(-c4nc5ccccc5n4-c4ccccc4)c2)nc2ccccc23)cc1 ; c1ccc(N(c2ccccc2)c2cc3ccccc3c3c2c(-c2ccc(-c4ccc5ccc6cccnc6c5n4)cc2)nc2ccccc23)cc1]

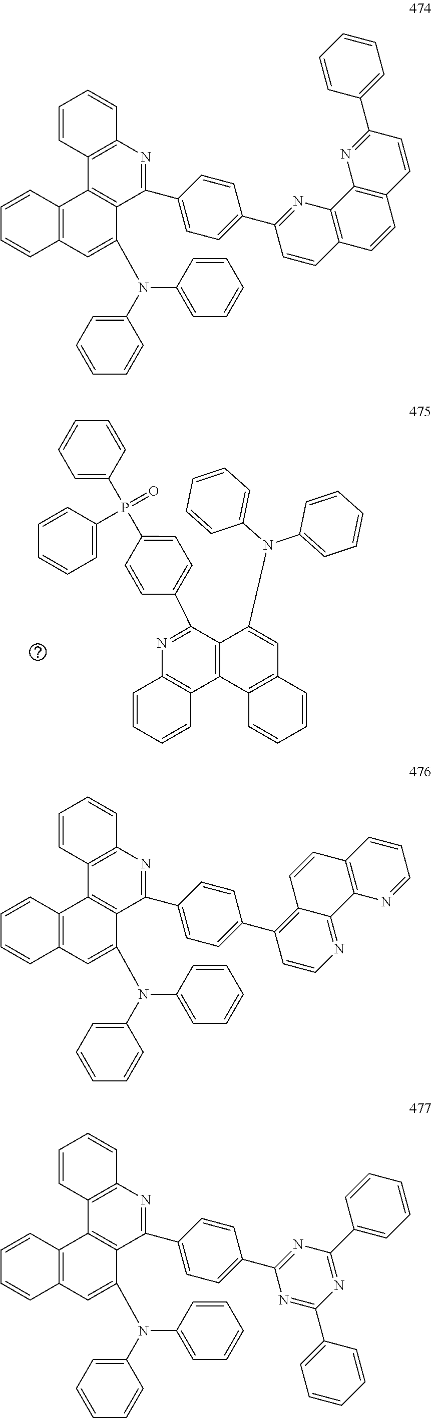 [SMILES: c1ccc(-c2ccc3ccc4ccc(-c5ccc(-c6nc7ccccc7c7c6c(N(c6ccccc6)c6ccccc6)cc6ccccc67)cc5)nc4c3n2)cc1 ; O=P(c1ccccc1)(c1ccccc1)c1ccc(-c2nc3ccccc3c3c2c(N(c2ccccc2)c2ccccc2)cc2ccccc23)cc1 ; c1ccc(N(c2ccccc2)c2cc3ccccc3c3c2c(-c2ccc(-c4ccnc5c4ccc4cccnc45)cc2)nc2ccccc23)cc1 ; c1ccc(-c2nc(-c3ccccc3)nc(-c3ccc(-c4nc5ccccc5c5c4c(N(c4ccccc4)c4ccccc4)cc4ccccc45)cc3)n2)cc1]

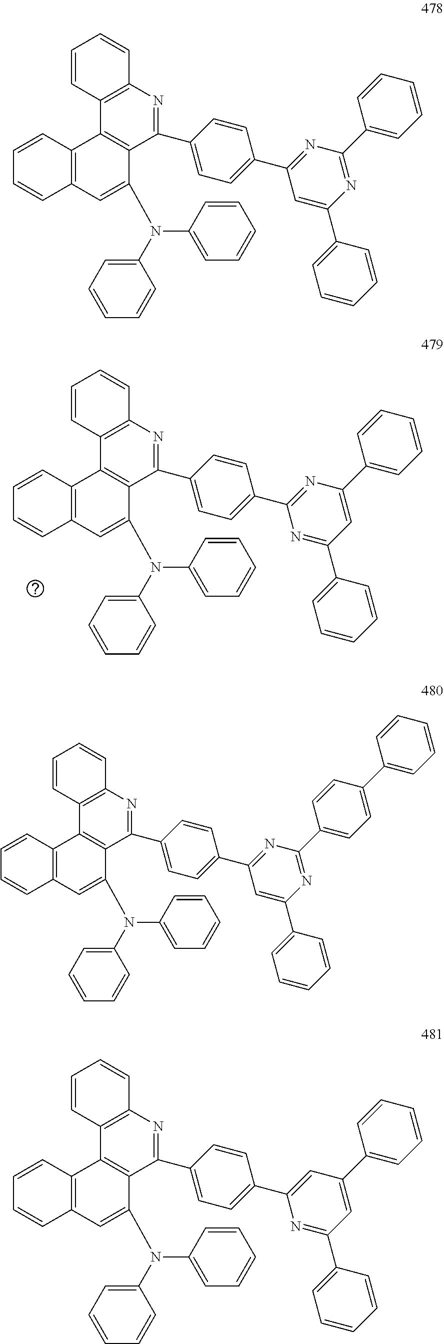 [SMILES: c1ccc(-c2cc(-c3ccc(-c4nc5ccccc5c5c4c(N(c4ccccc4)c4ccccc4)cc4ccccc45)cc3)nc(-c3ccccc3)n2)cc1 ; c1ccc(-c2cc(-c3ccccc3)nc(-c3ccc(-c4nc5ccccc5c5c4c(N(c4ccccc4)c4ccccc4)cc4ccccc45)cc3)n2)cc1 ; c1ccc(-c2ccc(-c3nc(-c4ccccc4)cc(-c4ccc(-c5nc6ccccc6c6c5c(N(c5ccccc5)c5ccccc5)cc5ccccc56)cc4)n3)cc2)cc1 ; c1ccc(-c2cc(-c3ccccc3)nc(-c3ccc(-c4nc5ccccc5c5c4c(N(c4ccccc4)c4ccccc4)cc4ccccc45)cc3)c2)cc1]

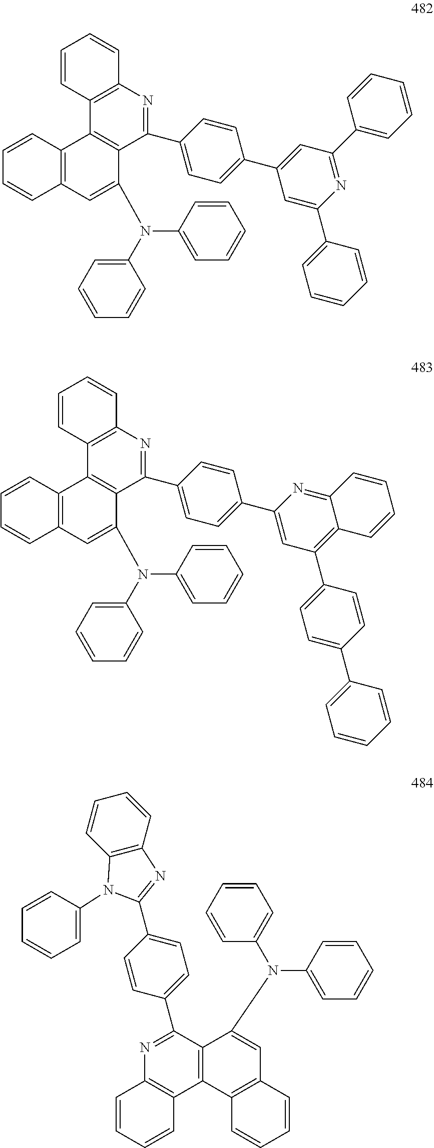 [SMILES: c1ccc(-c2cc(-c3ccc(-c4nc5ccccc5c5c4c(N(c4ccccc4)c4ccccc4)cc4ccccc45)cc3)cc(-c3ccccc3)n2)cc1 ; c1ccc(-c2ccc(-c3cc(-c4ccc(-c5nc6ccccc6c6c5c(N(c5ccccc5)c5ccccc5)cc5ccccc56)cc4)nc4ccccc34)cc2)cc1 ; c1ccc(N(c2ccccc2)c2cc3ccccc3c3c2c(-c2ccc(-c4nc5ccccc5n4-c4ccccc4)cc2)nc2ccccc23)cc1]

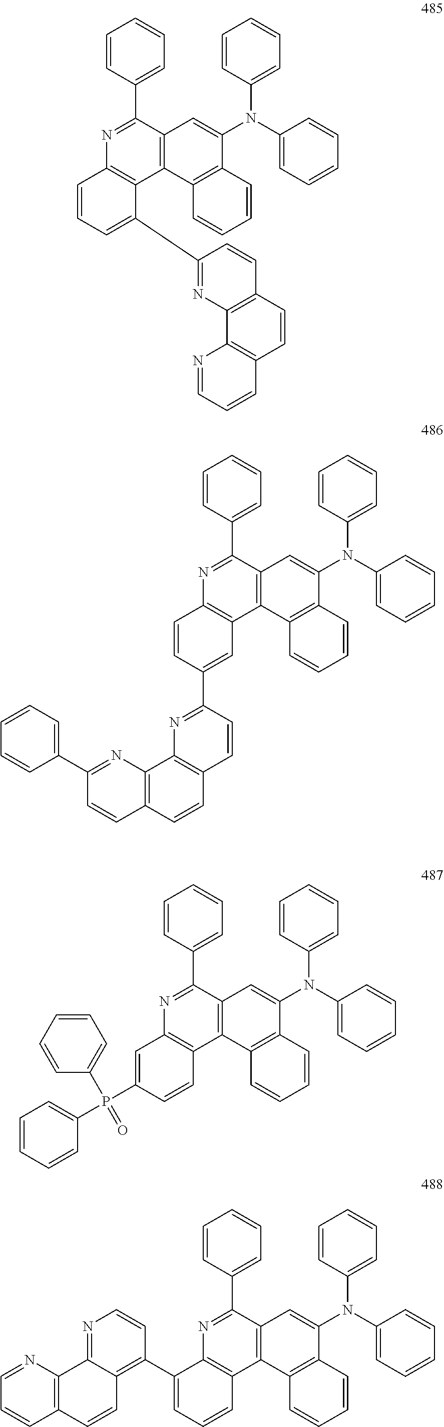

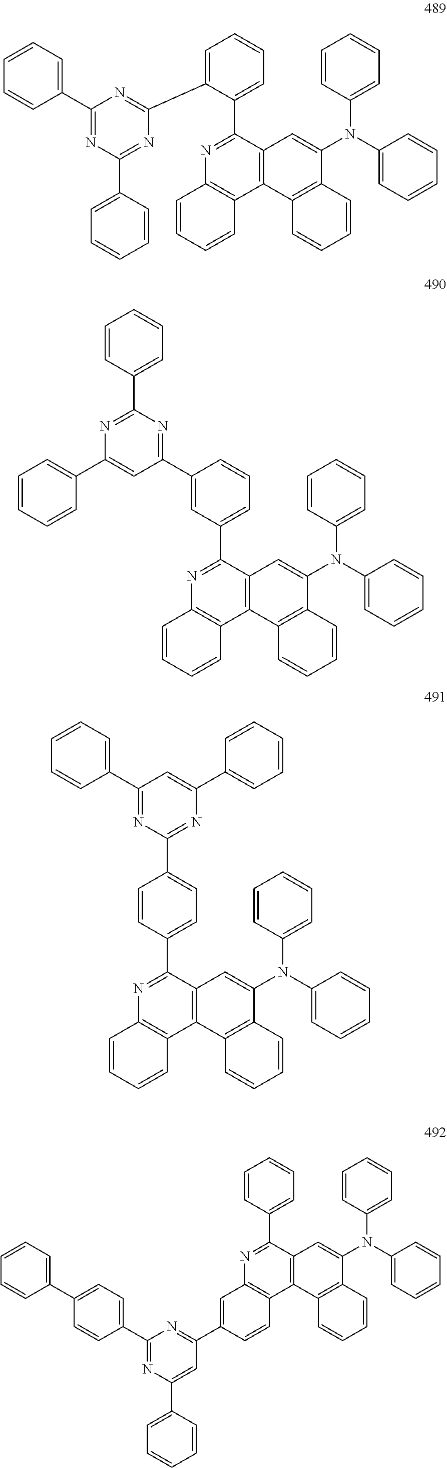 [SMILES: c1ccc(-c2nc(-c3ccccc3)nc(-c3ccccc3-c3nc4ccccc4c4c3cc(N(c3ccccc3)c3ccccc3)c3ccccc34)n2)cc1 ; c1ccc(-c2cc(-c3cccc(-c4nc5ccccc5c5c4cc(N(c4ccccc4)c4ccccc4)c4ccccc45)c3)nc(-c3ccccc3)n2)cc1 ; c1ccc(-c2cc(-c3ccccc3)nc(-c3ccc(-c4nc5ccccc5c5c4cc(N(c4ccccc4)c4ccccc4)c4ccccc45)cc3)n2)cc1 ; c1ccc(-c2ccc(-c3nc(-c4ccccc4)cc(-c4ccc5c(c4)nc(-c4ccccc4)c4cc(N(c6ccccc6)c6ccccc6)c6ccccc6c45)n3)cc2)cc1]

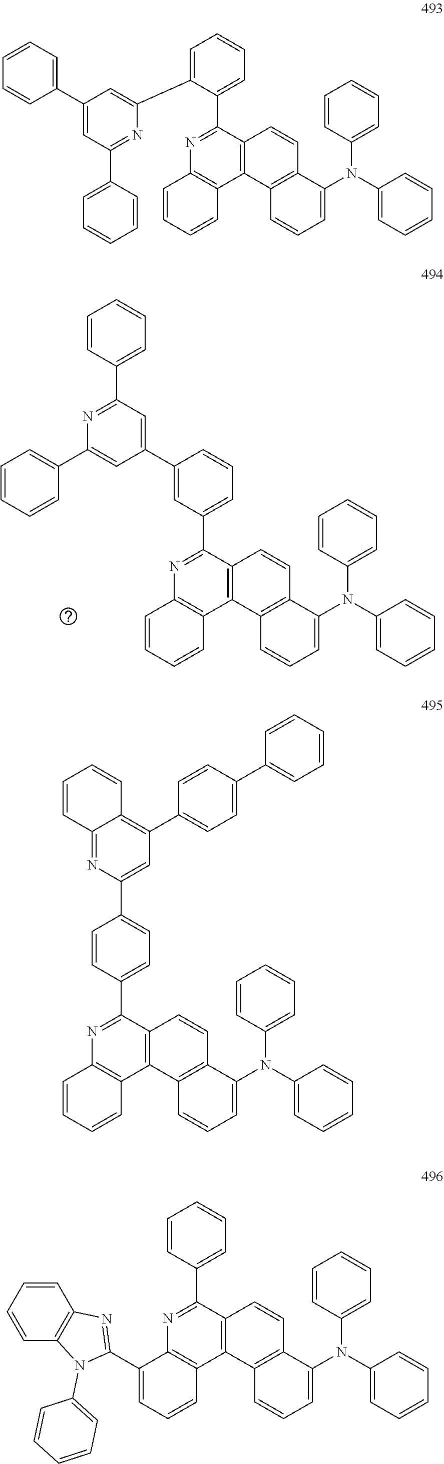 [SMILES: c1ccc(-c2cc(-c3ccccc3)nc(-c3ccccc3-c3nc4ccccc4c4c3ccc3c(N(c5ccccc5)c5ccccc5)cccc34)c2)cc1 ; c1ccc(-c2cc(-c3cccc(-c4nc5ccccc5c5c4ccc4c(N(c6ccccc6)c6ccccc6)cccc45)c3)cc(-c3ccccc3)n2)cc1 ; c1ccc(-c2ccc(-c3cc(-c4ccc(-c5nc6ccccc6c6c5ccc5c(N(c7ccccc7)c7ccccc7)cccc56)cc4)nc4ccccc34)cc2)cc1 ; c1ccc(-c2nc3c(-c4nc5ccccc5n4-c4ccccc4)cccc3c3c2ccc2c(N(c4ccccc4)c4ccccc4)cccc23)cc1]

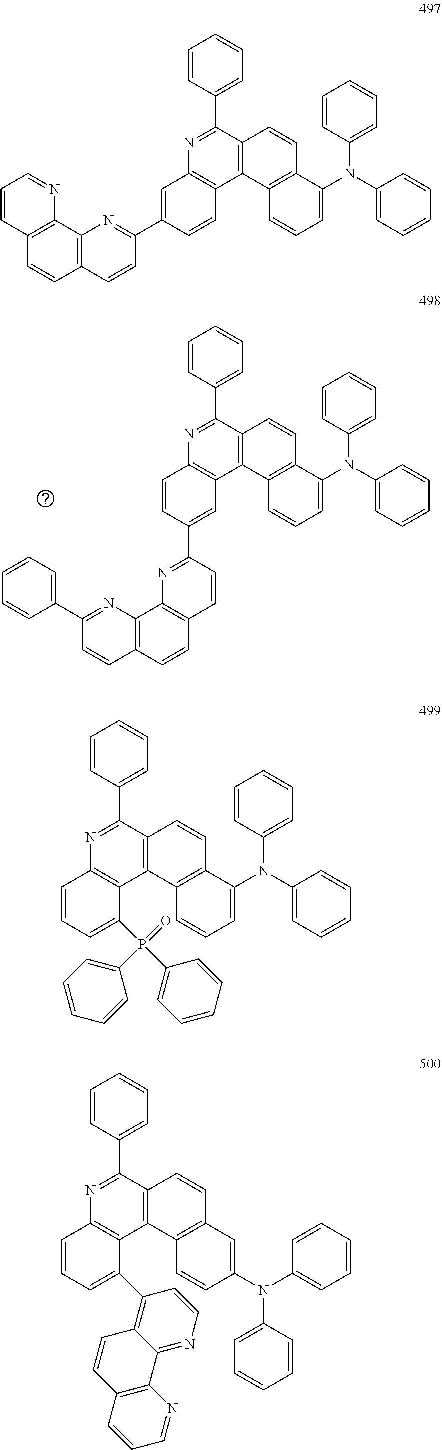 [SMILES: c1ccc(-c2nc3cc(-c4ccc5ccc6cccnc6c5n4)ccc3c3c2ccc2c(N(c4ccccc4)c4ccccc4)cccc23)cc1 ; c1ccc(-c2ccc3ccc4ccc(-c5ccc6nc(-c7ccccc7)c7ccc8c(N(c9ccccc9)c9ccccc9)cccc8c7c6c5)nc4c3n2)cc1 ; O=P(c1ccccc1)(c1ccccc1)c1cccc2nc(-c3ccccc3)c3ccc4c(N(c5ccccc5)c5ccccc5)cccc4c3c12 ; c1ccc(-c2nc3cccc(-c4ccnc5c4ccc4cccnc45)c3c3c2ccc2cc(N(c4ccccc4)c4ccccc4)ccc23)cc1]

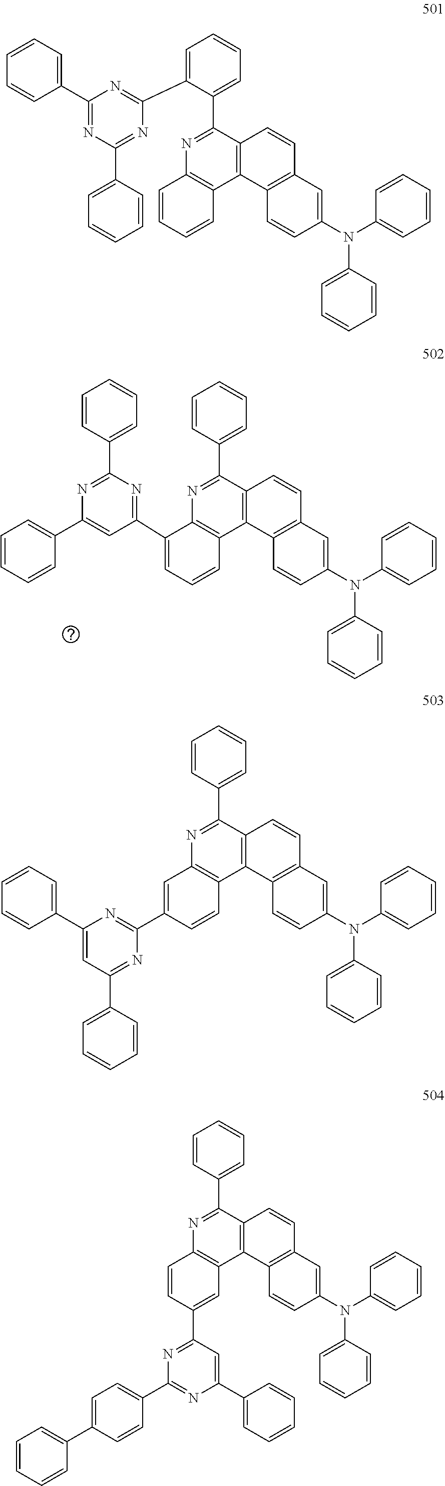 [SMILES: c1ccc(-c2nc(-c3ccccc3)nc(-c3ccccc3-c3nc4ccccc4c4c3ccc3cc(N(c5ccccc5)c5ccccc5)ccc34)n2)cc1 ; c1ccc(-c2cc(-c3cccc4c3nc(-c3ccccc3)c3ccc5cc(N(c6ccccc6)c6ccccc6)ccc5c34)nc(-c3ccccc3)n2)cc1 ; c1ccc(-c2cc(-c3ccccc3)nc(-c3ccc4c(c3)nc(-c3ccccc3)c3ccc5cc(N(c6ccccc6)c6ccccc6)ccc5c34)n2)cc1 ; c1ccc(-c2ccc(-c3nc(-c4ccccc4)cc(-c4ccc5nc(-c6ccccc6)c6ccc7cc(N(c8ccccc8)c8ccccc8)ccc7c6c5c4)n3)cc2)cc1]

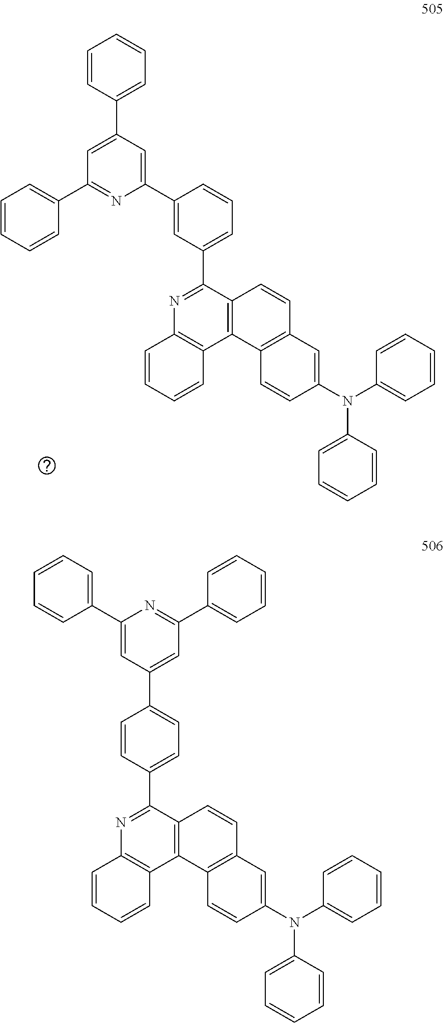 [SMILES: c1ccc(-c2cc(-c3ccccc3)nc(-c3cccc(-c4nc5ccccc5c5c4ccc4cc(N(c6ccccc6)c6ccccc6)ccc45)c3)c2)cc1 ; c1ccc(-c2cc(-c3ccc(-c4nc5ccccc5c5c4ccc4cc(N(c6ccccc6)c6ccccc6)ccc45)cc3)cc(-c3ccccc3)n2)cc1]

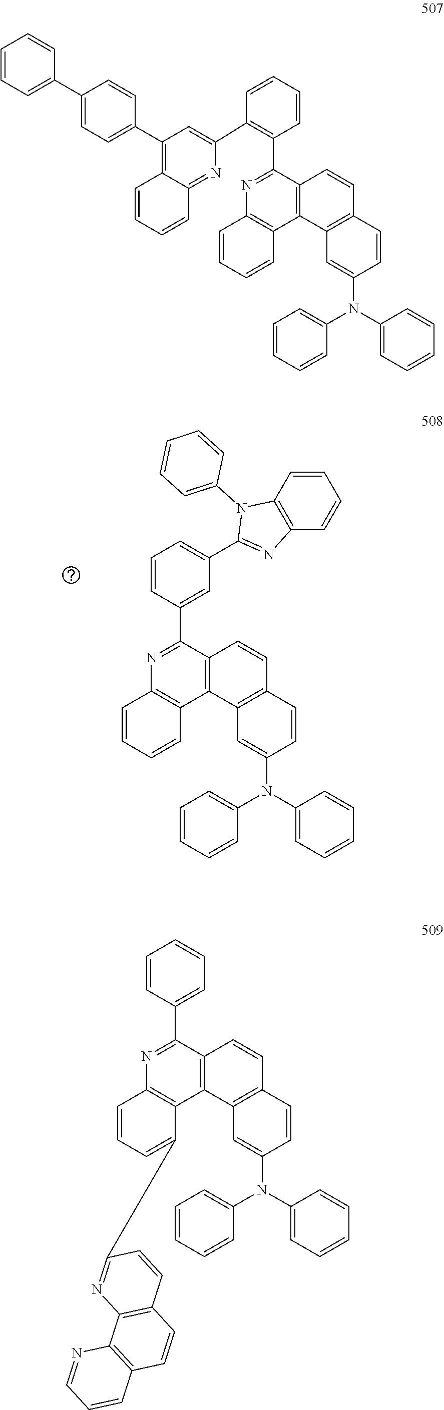 [SMILES: c1ccc(-c2ccc(-c3cc(-c4ccccc4-c4nc5ccccc5c5c4ccc4ccc(N(c6ccccc6)c6ccccc6)cc45)nc4ccccc34)cc2)cc1 ; c1ccc(N(c2ccccc2)c2ccc3ccc4c(-c5cccc(-c6nc7ccccc7n6-c6ccccc6)c5)nc5ccccc5c4c3c2)cc1 ; c1ccc(-c2nc3cccc(-c4ccc5ccc6cccnc6c5n4)c3c3c2ccc2ccc(N(c4ccccc4)c4ccccc4)cc23)cc1]

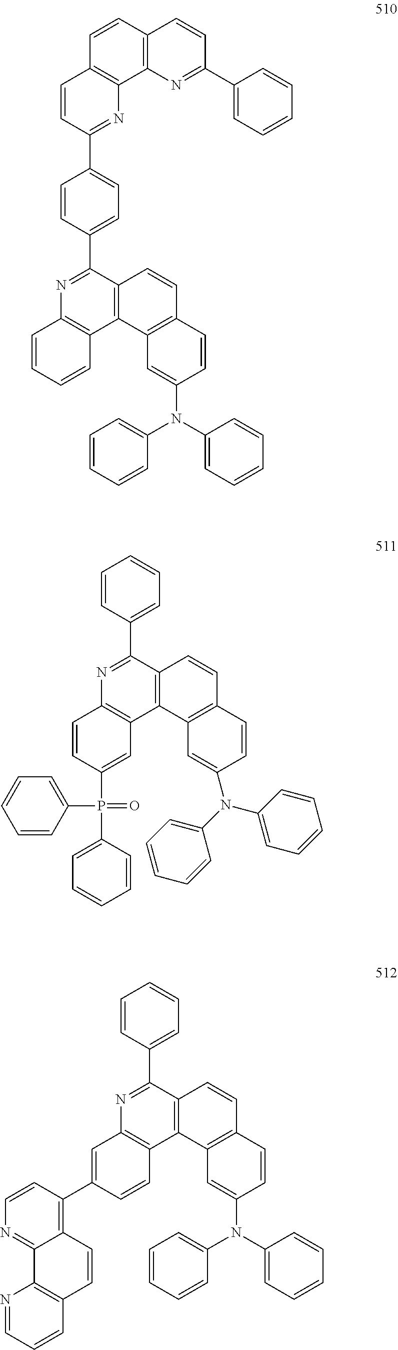 [SMILES: c1ccc(-c2ccc3ccc4ccc(-c5ccc(-c6nc7ccccc7c7c6ccc6ccc(N(c8ccccc8)c8ccccc8)cc67)cc5)nc4c3n2)cc1 ; O=P(c1ccccc1)(c1ccccc1)c1ccc2nc(-c3ccccc3)c3ccc4ccc(N(c5ccccc5)c5ccccc5)cc4c3c2c1 ; c1ccc(-c2nc3cc(-c4ccnc5c4ccc4cccnc45)ccc3c3c2ccc2ccc(N(c4ccccc4)c4ccccc4)cc23)cc1]

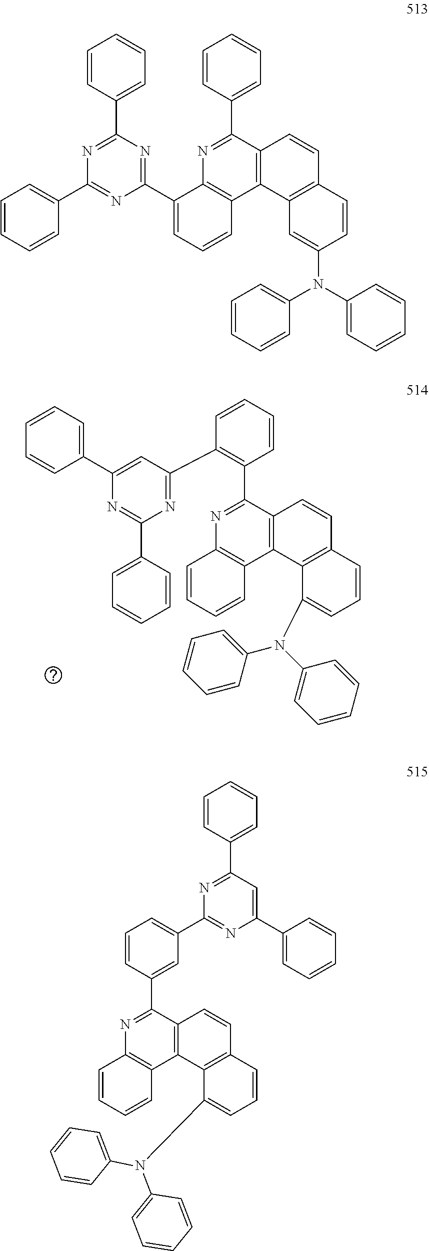 [SMILES: c1ccc(-c2nc(-c3ccccc3)nc(-c3cccc4c3nc(-c3ccccc3)c3ccc5ccc(N(c6ccccc6)c6ccccc6)cc5c34)n2)cc1 ; c1ccc(-c2cc(-c3ccccc3-c3nc4ccccc4c4c3ccc3cccc(N(c5ccccc5)c5ccccc5)c34)nc(-c3ccccc3)n2)cc1 ; c1ccc(-c2cc(-c3ccccc3)nc(-c3cccc(-c4nc5ccccc5c5c4ccc4cccc(N(c6ccccc6)c6ccccc6)c45)c3)n2)cc1]

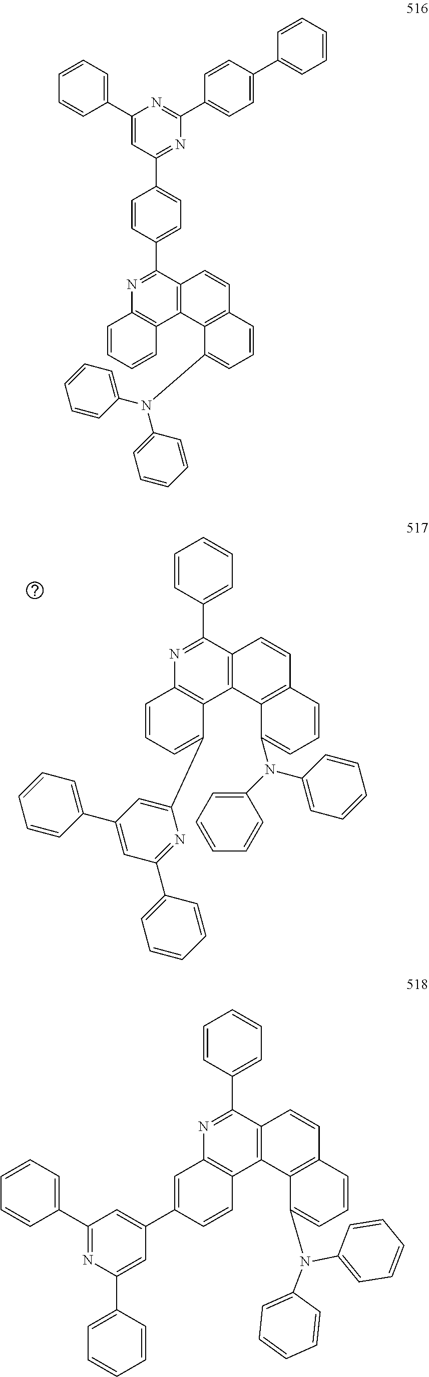 [SMILES: c1ccc(-c2ccc(-c3nc(-c4ccccc4)cc(-c4ccc(-c5nc6ccccc6c6c5ccc5cccc(N(c7ccccc7)c7ccccc7)c56)cc4)n3)cc2)cc1 ; c1ccc(-c2cc(-c3ccccc3)nc(-c3cccc4nc(-c5ccccc5)c5ccc6cccc(N(c7ccccc7)c7ccccc7)c6c5c34)c2)cc1 ; c1ccc(-c2cc(-c3ccc4c(c3)nc(-c3ccccc3)c3ccc5cccc(N(c6ccccc6)c6ccccc6)c5c34)cc(-c3ccccc3)n2)cc1]

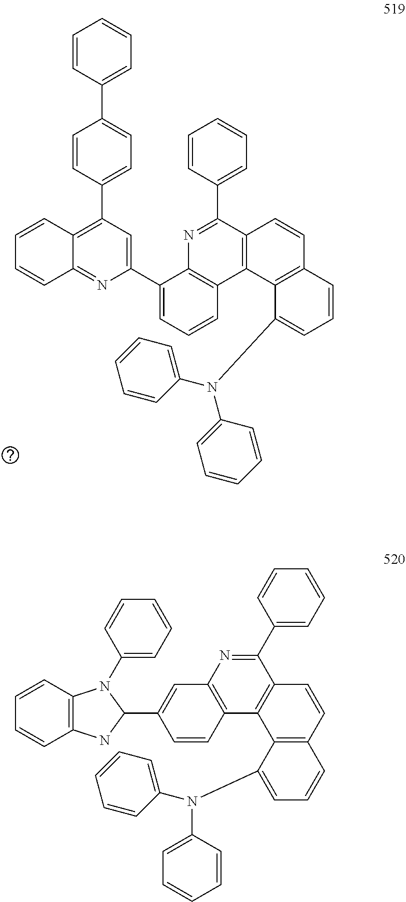

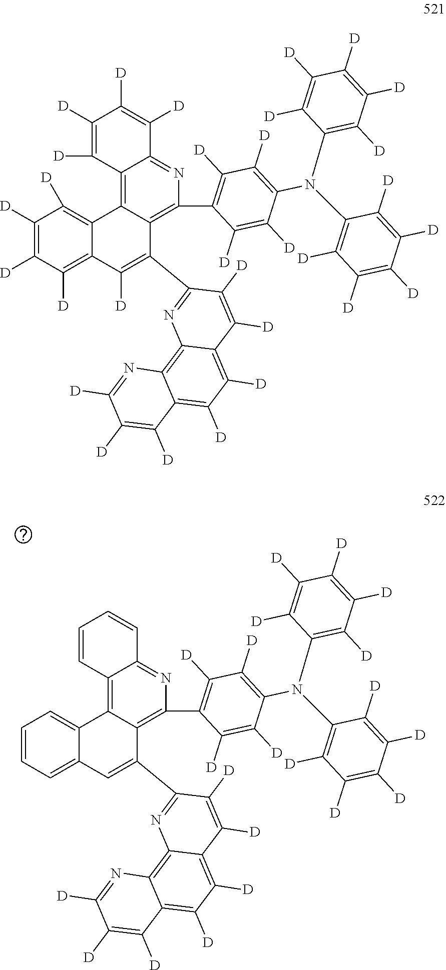 [SMILES: [2H]c1nc2c(c([2H])c1[2H])c([2H])c([2H])c1c([2H])c([2H])c(-c3c([2H])c4c([2H])c([2H])c([2H])c([2H])c4c4c3c(-c3c([2H])c([2H])c(N(c5c([2H])c([2H])c([2H])c([2H])c5[2H])c5c([2H])c([2H])c([2H])c([2H])c5[2H])c([2H])c3[2H])nc3c([2H])c([2H])c([2H])c([2H])c34)nc12 ; [2H]c1nc2c(c([2H])c1[2H])c([2H])c([2H])c1c([2H])c([2H])c(-c3cc4ccccc4c4c3c(-c3c([2H])c([2H])c(N(c5c([2H])c([2H])c([2H])c([2H])c5[2H])c5c([2H])c([2H])c([2H])c([2H])c5[2H])c([2H])c3[2H])nc3ccccc34)nc12]

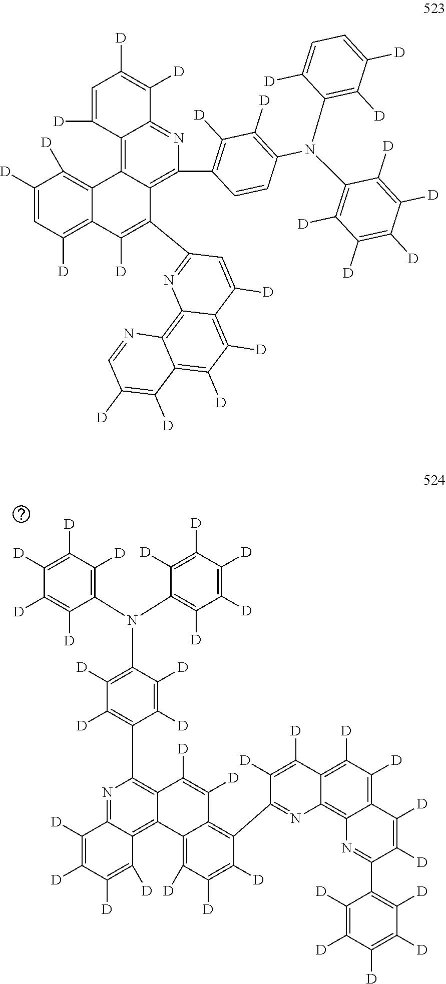 [SMILES: [2H]c1ccc([2H])c(N(c2ccc(-c3nc4c([2H])c([2H])cc([2H])c4c4c3c(-c3cc([2H])c5c([2H])c([2H])c6c([2H])c([2H])cnc6c5n3)c([2H])c3c([2H])cc([2H])c([2H])c34)c([2H])c2[2H])c2c([2H])c([2H])c([2H])c([2H])c2[2H])c1[2H] ; [2H]c1c([2H])c([2H])c(-c2nc3c(c([2H])c2[2H])c([2H])c([2H])c2c([2H])c([2H])c(-c4c([2H])c([2H])c([2H])c5c4c([2H])c([2H])c4c(-c6c([2H])c([2H])c(N(c7c([2H])c([2H])c([2H])c([2H])c7[2H])c7c([2H])c([2H])c([2H])c([2H])c7[2H])c([2H])c6[2H])nc6c([2H])c([2H])c([2H])c([2H])c6c45)nc23)c([2H])c1[2H]]

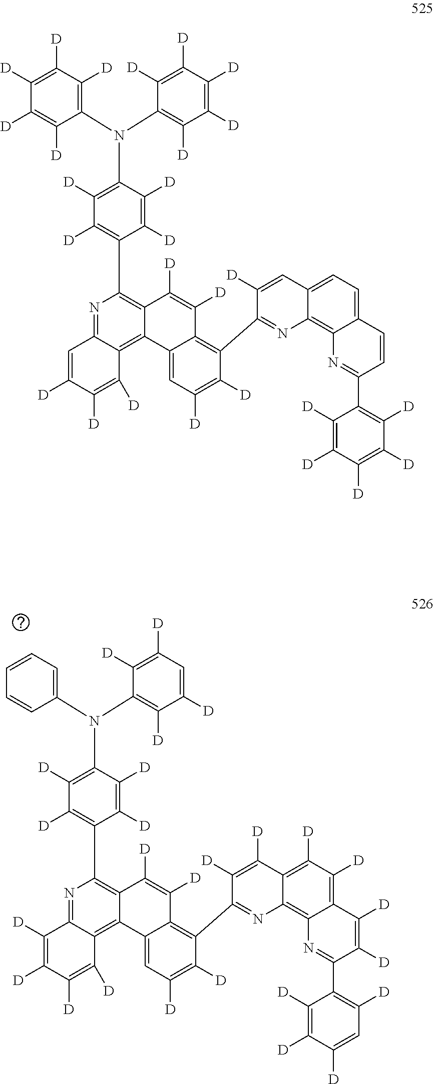 [SMILES: [2H]c1cc2ccc3ccc(-c4c([2H])c([2H])c([2H])c([2H])c4[2H])nc3c2nc1-c1c([2H])c([2H])cc2c1c([2H])c([2H])c1c(-c3c([2H])c([2H])c(N(c4c([2H])c([2H])c([2H])c([2H])c4[2H])c4c([2H])c([2H])c([2H])c([2H])c4[2H])c([2H])c3[2H])nc3cc([2H])c([2H])c([2H])c3c12 ; [2H]c1cc([2H])c(-c2nc3c(c([2H])c2[2H])c([2H])c([2H])c2c([2H])c([2H])c(-c4c([2H])c([2H])cc5c4c([2H])c([2H])c4c(-c6c([2H])c([2H])c(N(c7ccccc7)c7c([2H])c([2H])cc([2H])c7[2H])c([2H])c6[2H])nc6c([2H])c([2H])c([2H])c([2H])c6c45)nc23)c([2H])c1[2H]]

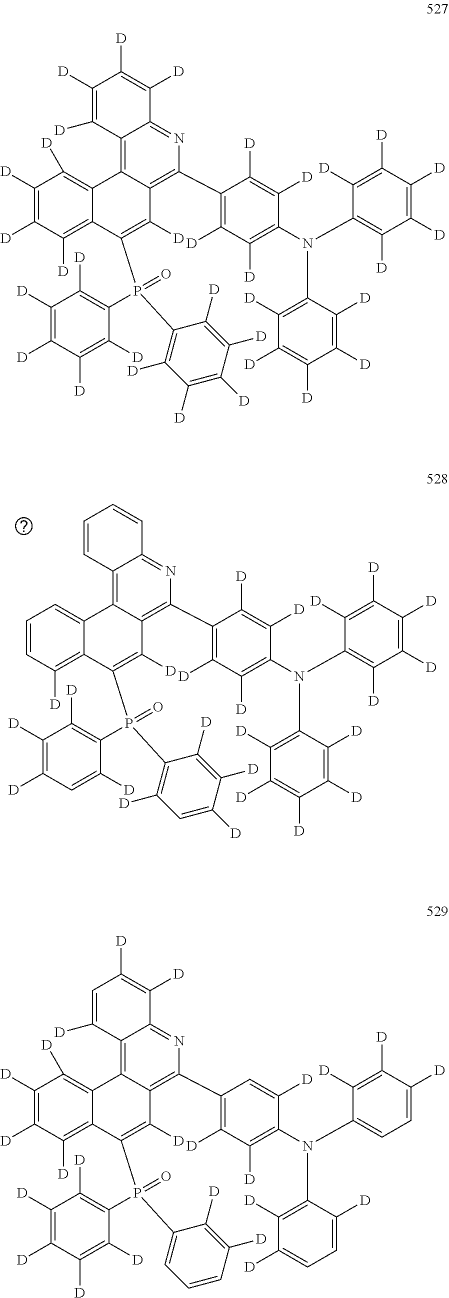

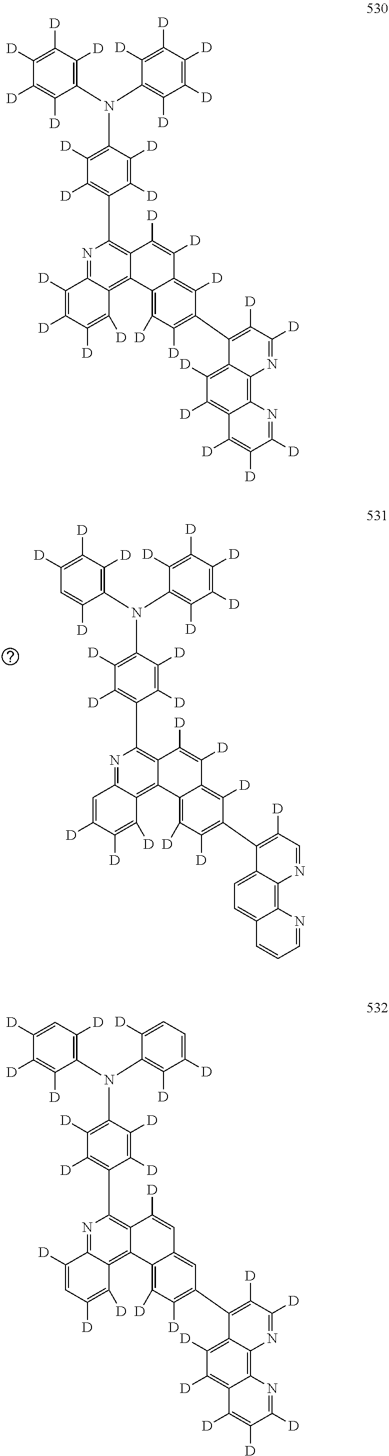

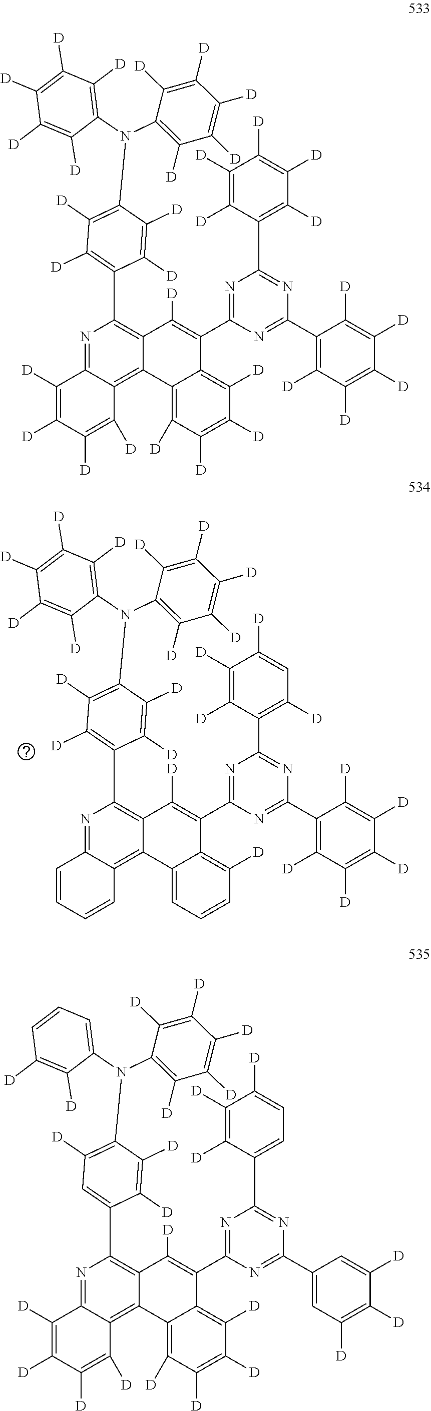

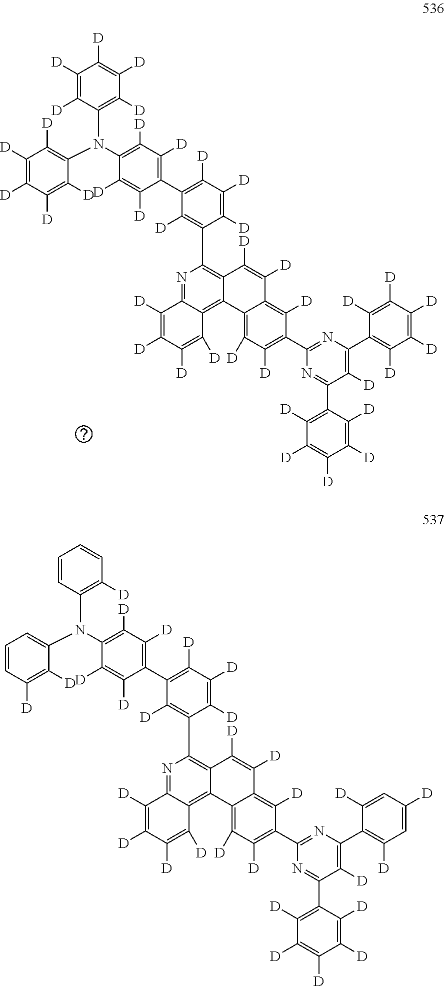

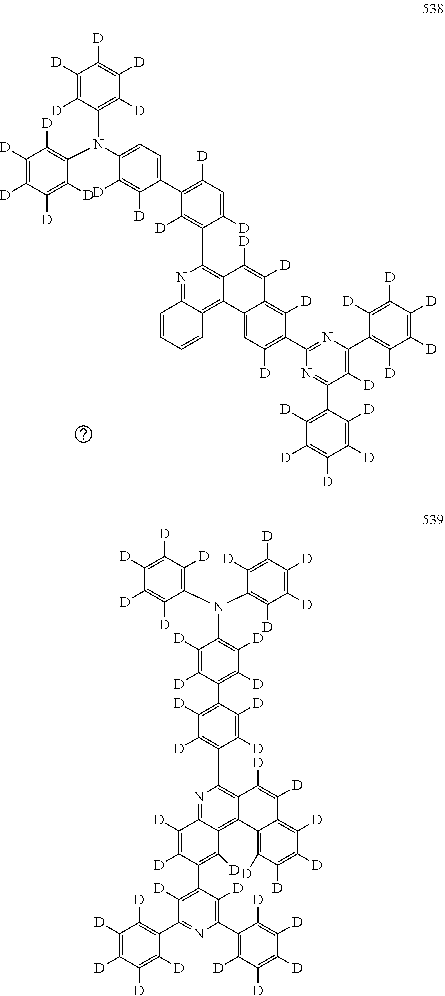

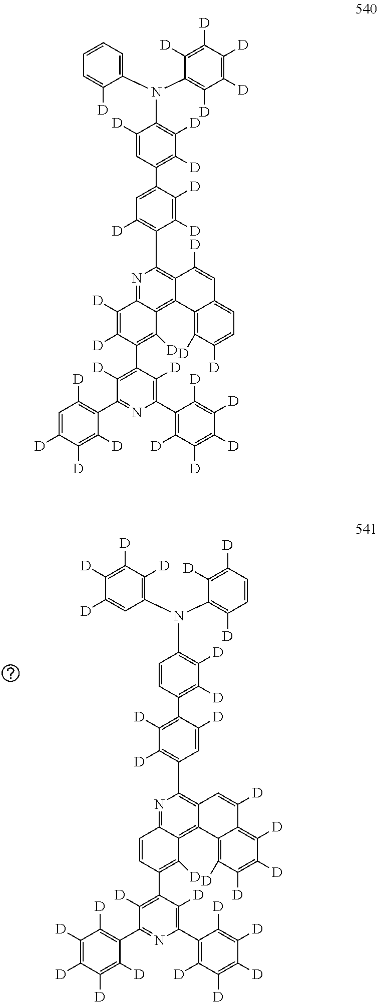

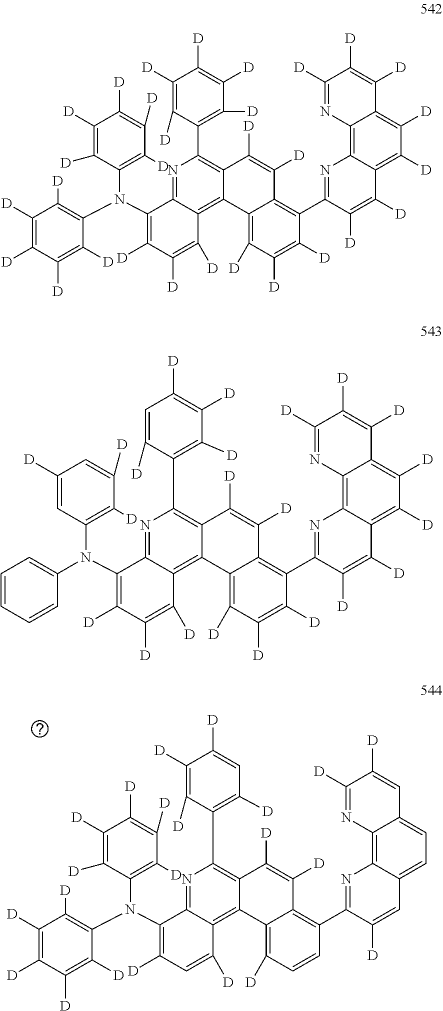

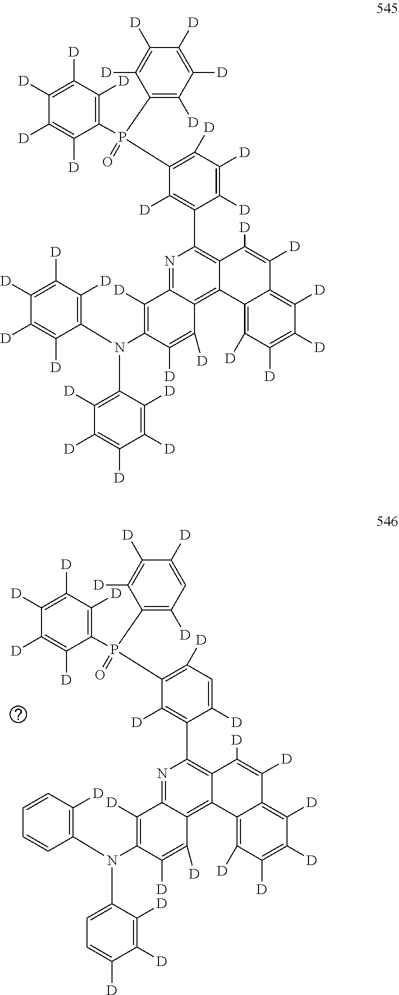 [SMILES: [2H]c1c([2H])c([2H])c(N(c2c([2H])c([2H])c([2H])c([2H])c2[2H])c2c([2H])c([2H])c3c(nc(-c4c([2H])c([2H])c([2H])c(P(=O)(c5c([2H])c([2H])c([2H])c([2H])c5[2H])c5c([2H])c([2H])c([2H])c([2H])c5[2H])c4[2H])c4c([2H])c([2H])c5c([2H])c([2H])c([2H])c([2H])c5c43)c2[2H])c([2H])c1[2H] ; [2H]c1ccccc1N(c1ccc([2H])c([2H])c1[2H])c1c([2H])c([2H])c2c(nc(-c3c([2H])cc([2H])c(P(=O)(c4c([2H])cc([2H])c([2H])c4[2H])c4c([2H])c([2H])c([2H])c([2H])c4[2H])c3[2H])c3c([2H])c([2H])c4c([2H])c([2H])c([2H])c([2H])c4c32)c1[2H]]

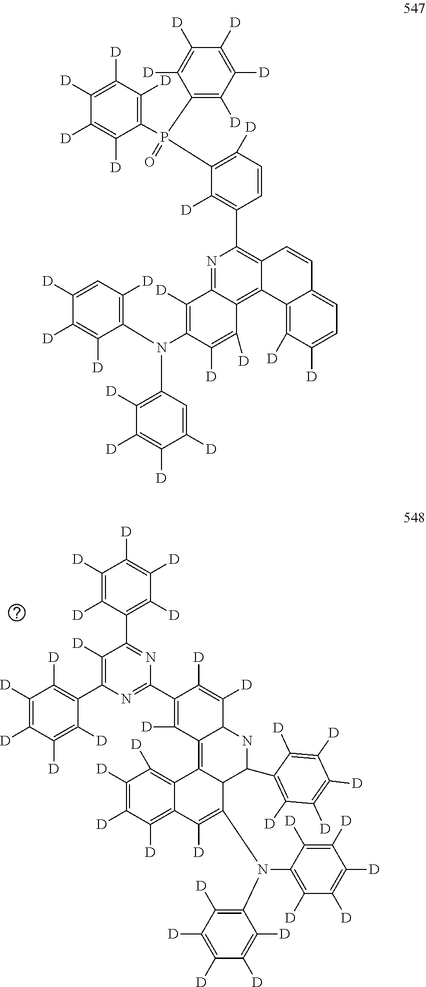

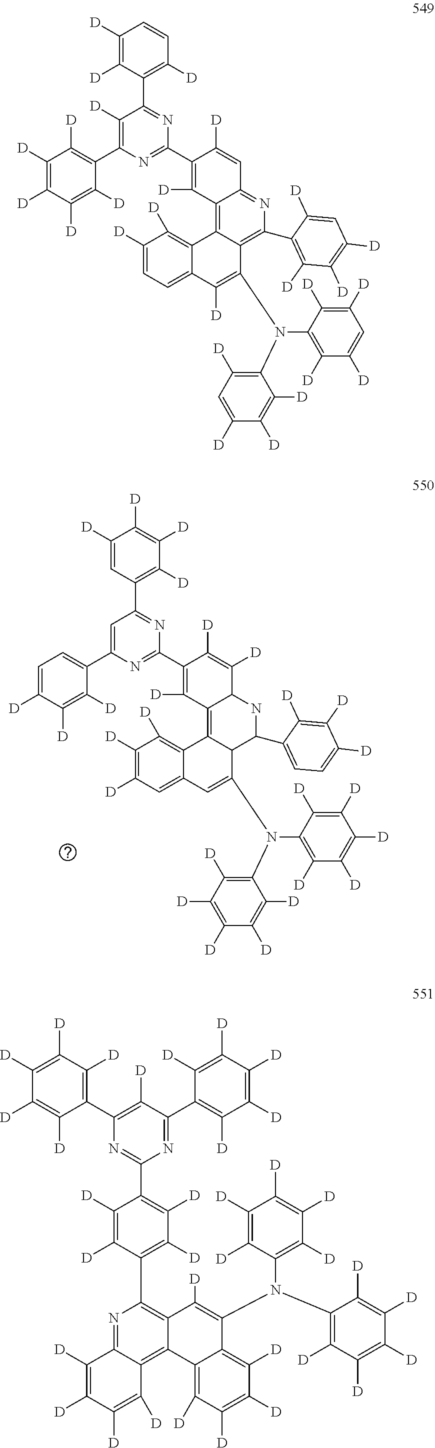

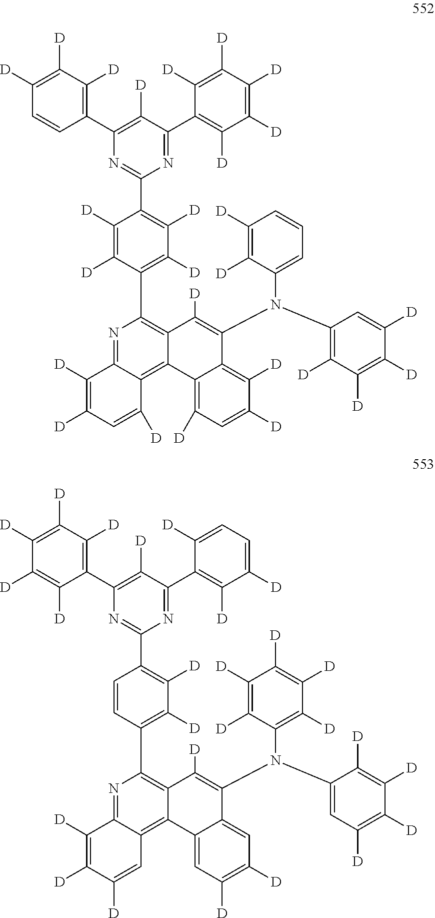 [SMILES: [2H]c1cccc(N(c2cc([2H])c([2H])c([2H])c2[2H])c2c([2H])c3c(-c4c([2H])c([2H])c(-c5nc(-c6ccc([2H])c([2H])c6[2H])c([2H])c(-c6c([2H])c([2H])c([2H])c([2H])c6[2H])n5)c([2H])c4[2H])nc4c([2H])c([2H])cc([2H])c4c3c3c([2H])cc([2H])c([2H])c23)c1[2H] ; [2H]c1cc2c(N(c3cc([2H])c([2H])c([2H])c3[2H])c3c([2H])c([2H])c([2H])c([2H])c3[2H])c([2H])c3c(-c4ccc(-c5nc(-c6c([2H])ccc([2H])c6[2H])c([2H])c(-c6c([2H])c([2H])c([2H])c([2H])c6[2H])n5)c([2H])c4[2H])nc4c([2H])c([2H])c([2H])cc4c3c2cc1[2H]]

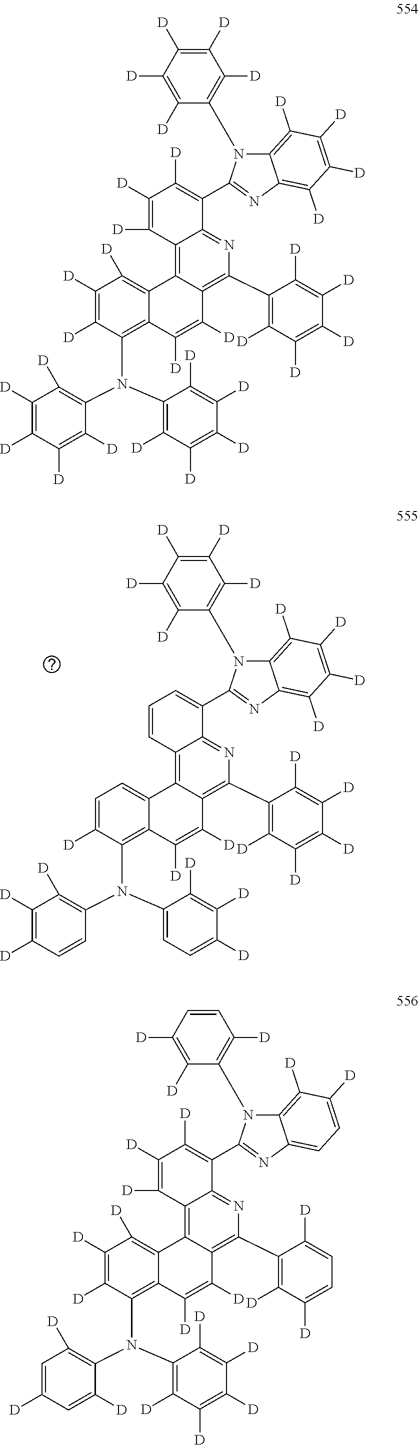

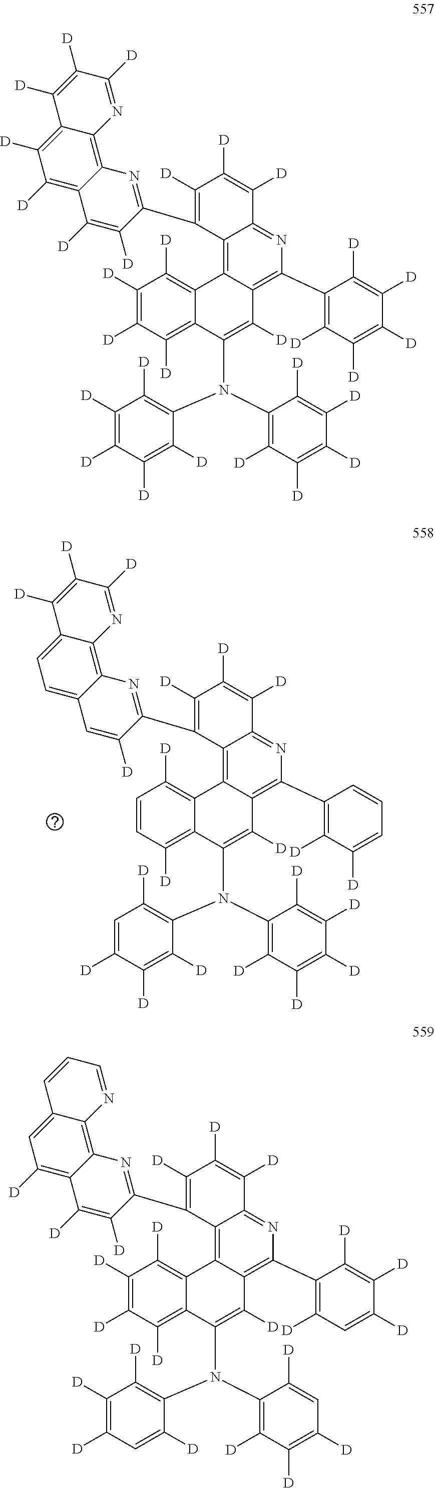

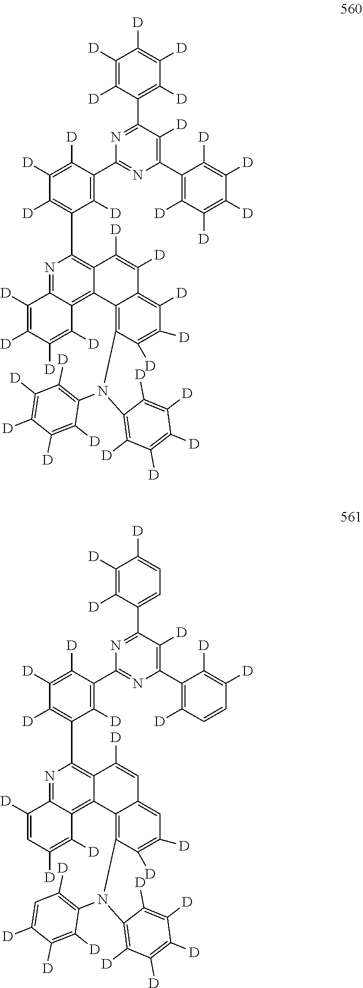

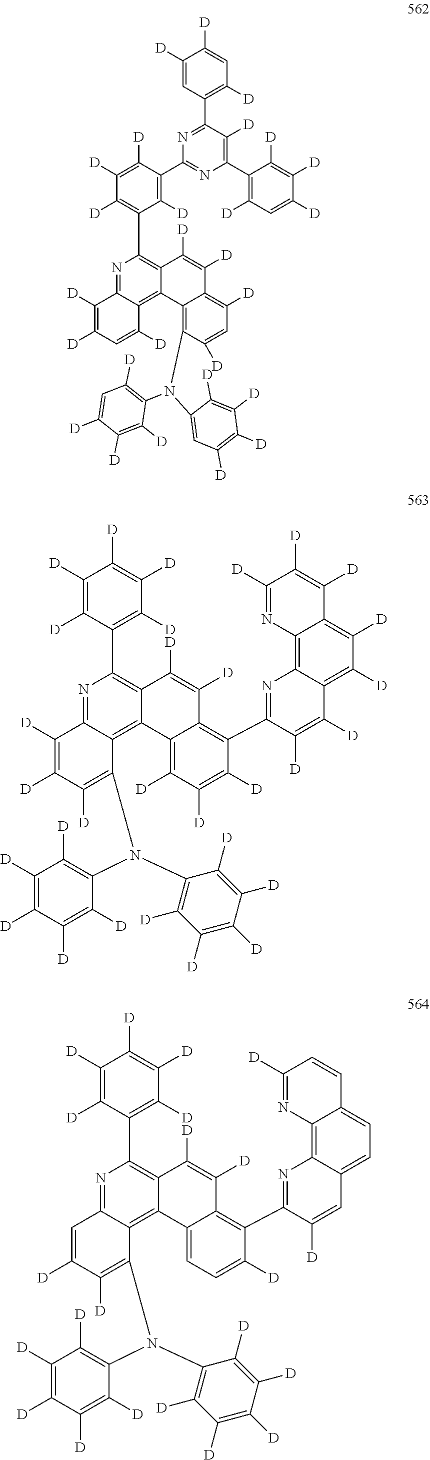

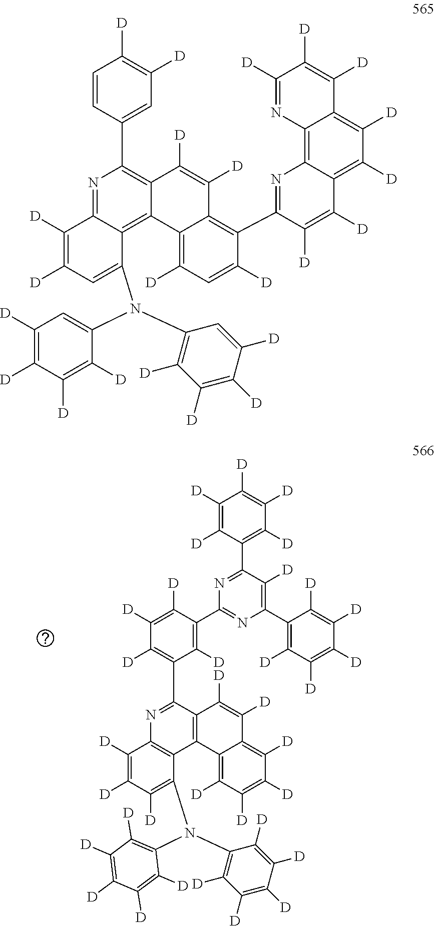 [SMILES: [2H]c1ccc(-c2nc3c([2H])c([2H])cc(N(c4cc([2H])c([2H])c([2H])c4[2H])c4cc([2H])c([2H])c([2H])c4[2H])c3c3c2c([2H])c([2H])c2c(-c4nc5c(c([2H])c4[2H])c([2H])c([2H])c4c([2H])c([2H])c([2H])nc45)c([2H])cc([2H])c23)cc1[2H] ; [2H]c1c(-c2c([2H])c([2H])c([2H])c([2H])c2[2H])nc(-c2c([2H])c([2H])c([2H])c(-c3nc4c([2H])c([2H])c([2H])c(N(c5c([2H])c([2H])c([2H])c([2H])c5[2H])c5c([2H])c([2H])c([2H])c([2H])c5[2H])c4c4c3c([2H])c([2H])c3c([2H])c([2H])c([2H])c([2H])c34)c2[2H])nc1-c1c([2H])c([2H])c([2H])c([2H])c1[2H]]

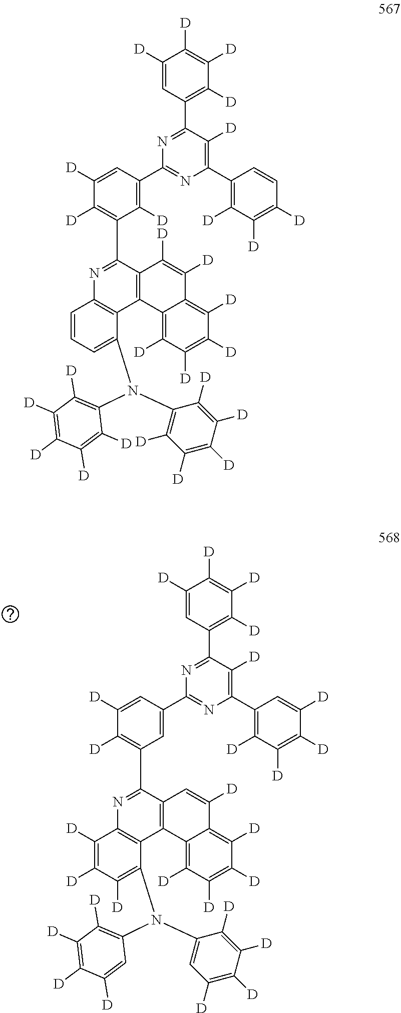 [SMILES: [2H]c1ccc(-c2nc(-c3cc([2H])c([2H])c(-c4nc5cccc(N(c6c([2H])c([2H])c([2H])c([2H])c6[2H])c6c([2H])c([2H])c([2H])c([2H])c6[2H])c5c5c4c([2H])c([2H])c4c([2H])c([2H])c([2H])c([2H])c45)c3[2H])nc(-c3cc([2H])c([2H])c([2H])c3[2H])c2[2H])c([2H])c1[2H] ; [2H]c1cc(-c2nc(-c3cc([2H])c([2H])c([2H])c3[2H])c([2H])c(-c3cc([2H])c([2H])c([2H])c3[2H])n2)cc(-c2nc3c([2H])c([2H])c([2H])c(N(c4cc([2H])c([2H])c([2H])c4[2H])c4cc([2H])c([2H])c([2H])c4[2H])c3c3c2cc([2H])c2c([2H])c([2H])c([2H])c([2H])c23)c1[2H]]

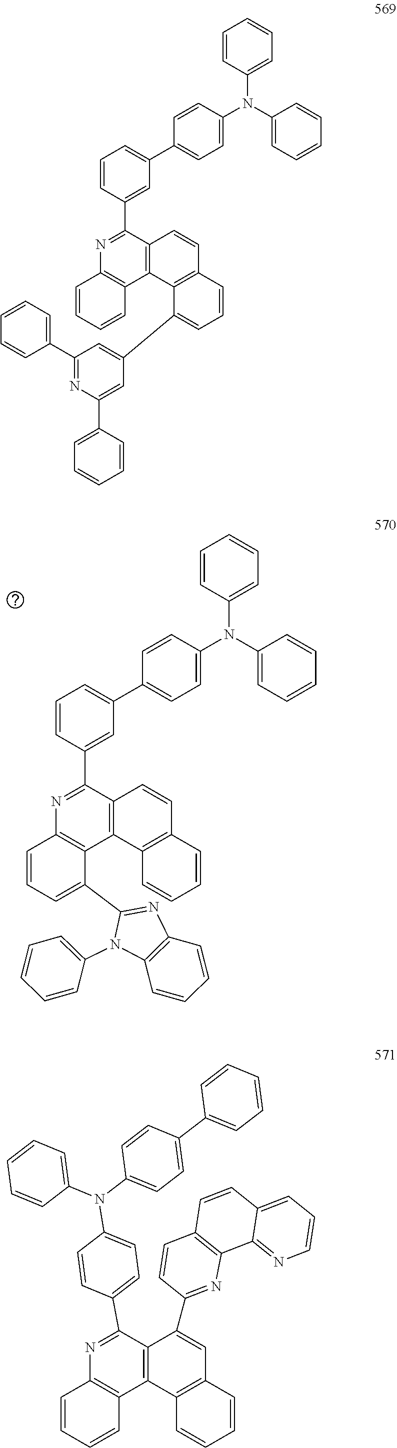 [SMILES: c1ccc(-c2cc(-c3cccc4ccc5c(-c6cccc(-c7ccc(N(c8ccccc8)c8ccccc8)cc7)c6)nc6ccccc6c5c34)cc(-c3ccccc3)n2)cc1 ; c1ccc(N(c2ccccc2)c2ccc(-c3cccc(-c4nc5cccc(-c6nc7ccccc7n6-c6ccccc6)c5c5c4ccc4ccccc45)c3)cc2)cc1 ; c1ccc(-c2ccc(N(c3ccccc3)c3ccc(-c4nc5ccccc5c5c4c(-c4ccc6ccc7cccnc7c6n4)cc4ccccc45)cc3)cc2)cc1]

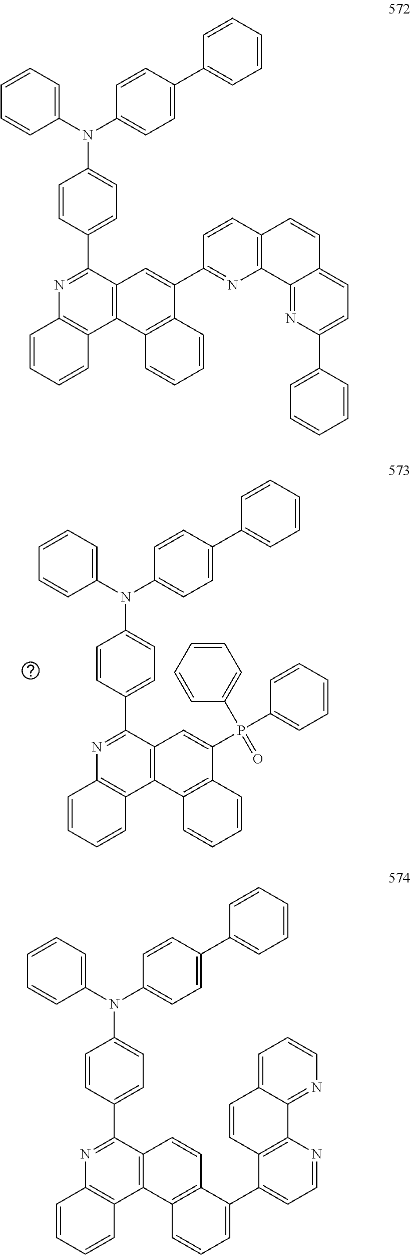 [SMILES: c1ccc(-c2ccc(N(c3ccccc3)c3ccc(-c4nc5ccccc5c5c4cc(-c4ccc6ccc7ccc(-c8ccccc8)nc7c6n4)c4ccccc45)cc3)cc2)cc1 ; O=P(c1ccccc1)(c1ccccc1)c1cc2c(-c3ccc(N(c4ccccc4)c4ccc(-c5ccccc5)cc4)cc3)nc3ccccc3c2c2ccccc12 ; c1ccc(-c2ccc(N(c3ccccc3)c3ccc(-c4nc5ccccc5c5c4ccc4c(-c6ccnc7c6ccc6cccnc67)cccc45)cc3)cc2)cc1]

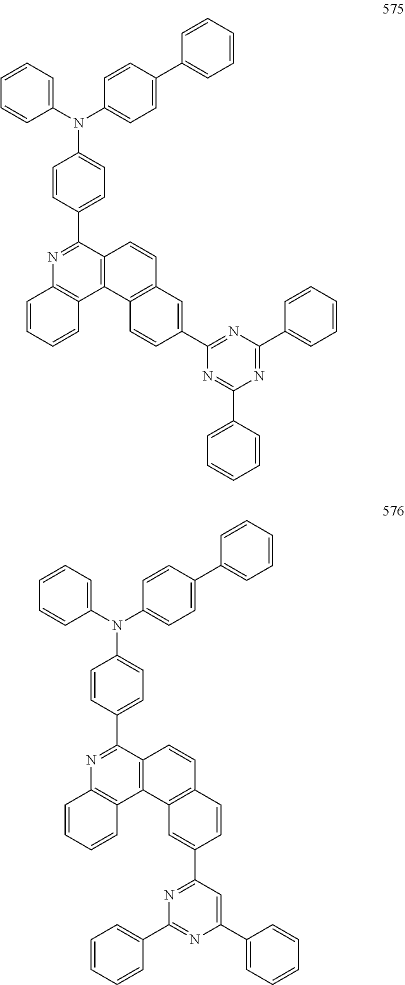 [SMILES: c1ccc(-c2ccc(N(c3ccccc3)c3ccc(-c4nc5ccccc5c5c4ccc4cc(-c6nc(-c7ccccc7)nc(-c7ccccc7)n6)ccc45)cc3)cc2)cc1 ; c1ccc(-c2ccc(N(c3ccccc3)c3ccc(-c4nc5ccccc5c5c4ccc4ccc(-c6cc(-c7ccccc7)nc(-c7ccccc7)n6)cc45)cc3)cc2)cc1]

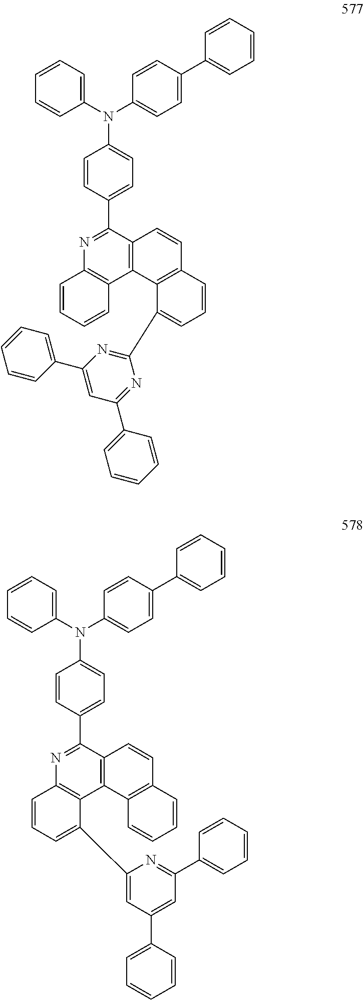 [SMILES: c1ccc(-c2ccc(N(c3ccccc3)c3ccc(-c4nc5ccccc5c5c4ccc4cccc(-c6nc(-c7ccccc7)cc(-c7ccccc7)n6)c45)cc3)cc2)cc1 ; c1ccc(-c2ccc(N(c3ccccc3)c3ccc(-c4nc5cccc(-c6cc(-c7ccccc7)cc(-c7ccccc7)n6)c5c5c4ccc4ccccc45)cc3)cc2)cc1]

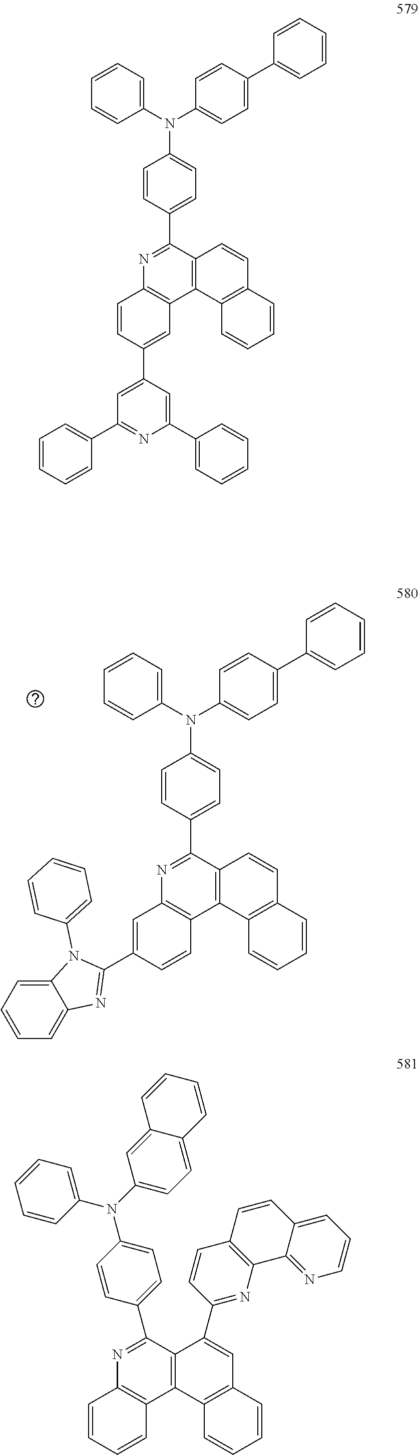 [SMILES: c1ccc(-c2ccc(N(c3ccccc3)c3ccc(-c4nc5ccc(-c6cc(-c7ccccc7)nc(-c7ccccc7)c6)cc5c5c4ccc4ccccc45)cc3)cc2)cc1 ; c1ccc(-c2ccc(N(c3ccccc3)c3ccc(-c4nc5cc(-c6nc7ccccc7n6-c6ccccc6)ccc5c5c4ccc4ccccc45)cc3)cc2)cc1 ; c1ccc(N(c2ccc(-c3nc4ccccc4c4c3c(-c3ccc5ccc6cccnc6c5n3)cc3ccccc34)cc2)c2ccc3ccccc3c2)cc1]

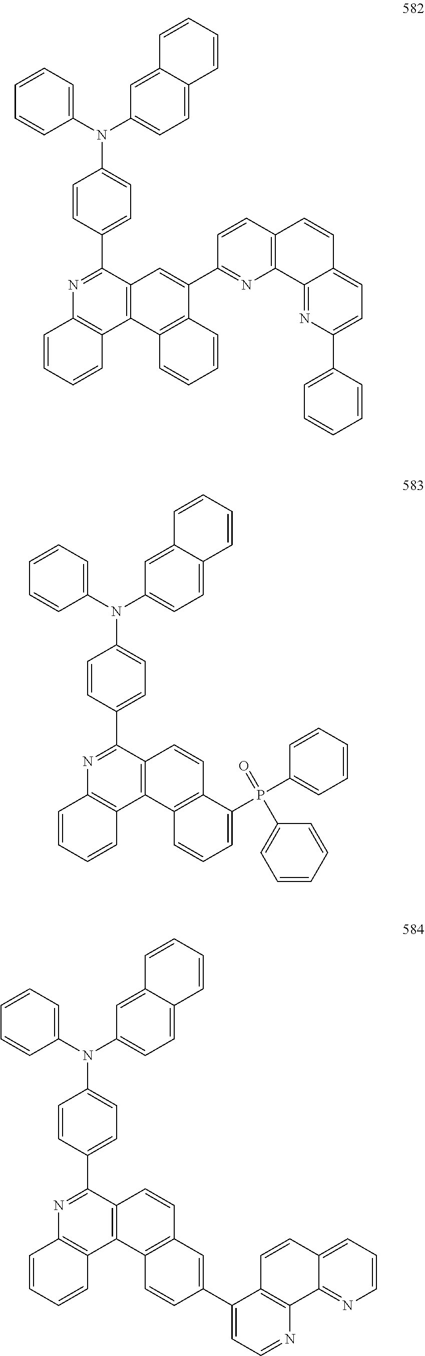 [SMILES: c1ccc(-c2ccc3ccc4ccc(-c5cc6c(-c7ccc(N(c8ccccc8)c8ccc9ccccc9c8)cc7)nc7ccccc7c6c6ccccc56)nc4c3n2)cc1 ; O=P(c1ccccc1)(c1ccccc1)c1cccc2c1ccc1c(-c3ccc(N(c4ccccc4)c4ccc5ccccc5c4)cc3)nc3ccccc3c12 ; c1ccc(N(c2ccc(-c3nc4ccccc4c4c3ccc3cc(-c5ccnc6c5ccc5cccnc56)ccc34)cc2)c2ccc3ccccc3c2)cc1]

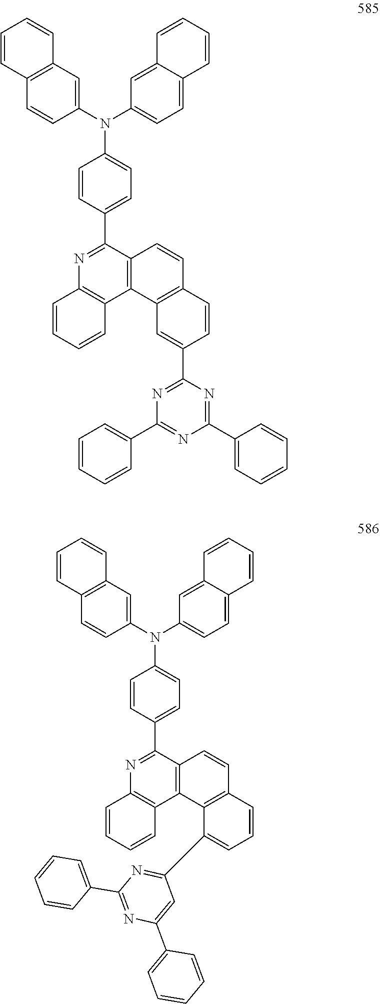 [SMILES: c1ccc(-c2nc(-c3ccccc3)nc(-c3ccc4ccc5c(-c6ccc(N(c7ccc8ccccc8c7)c7ccc8ccccc8c7)cc6)nc6ccccc6c5c4c3)n2)cc1 ; c1ccc(-c2cc(-c3cccc4ccc5c(-c6ccc(N(c7ccc8ccccc8c7)c7ccc8ccccc8c7)cc6)nc6ccccc6c5c34)nc(-c3ccccc3)n2)cc1]

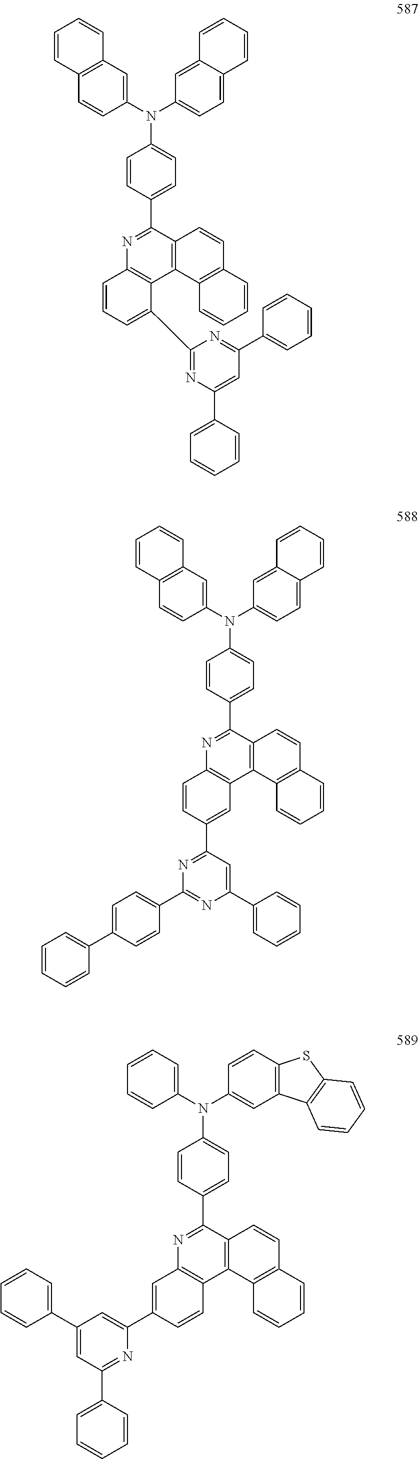 [SMILES: c1ccc(-c2cc(-c3ccccc3)nc(-c3cccc4nc(-c5ccc(N(c6ccc7ccccc7c6)c6ccc7ccccc7c6)cc5)c5ccc6ccccc6c5c34)n2)cc1 ; c1ccc(-c2ccc(-c3nc(-c4ccccc4)cc(-c4ccc5nc(-c6ccc(N(c7ccc8ccccc8c7)c7ccc8ccccc8c7)cc6)c6ccc7ccccc7c6c5c4)n3)cc2)cc1 ; c1ccc(-c2cc(-c3ccccc3)nc(-c3ccc4c(c3)nc(-c3ccc(N(c5ccccc5)c5ccc6sc7ccccc7c6c5)cc3)c3ccc5ccccc5c34)c2)cc1]

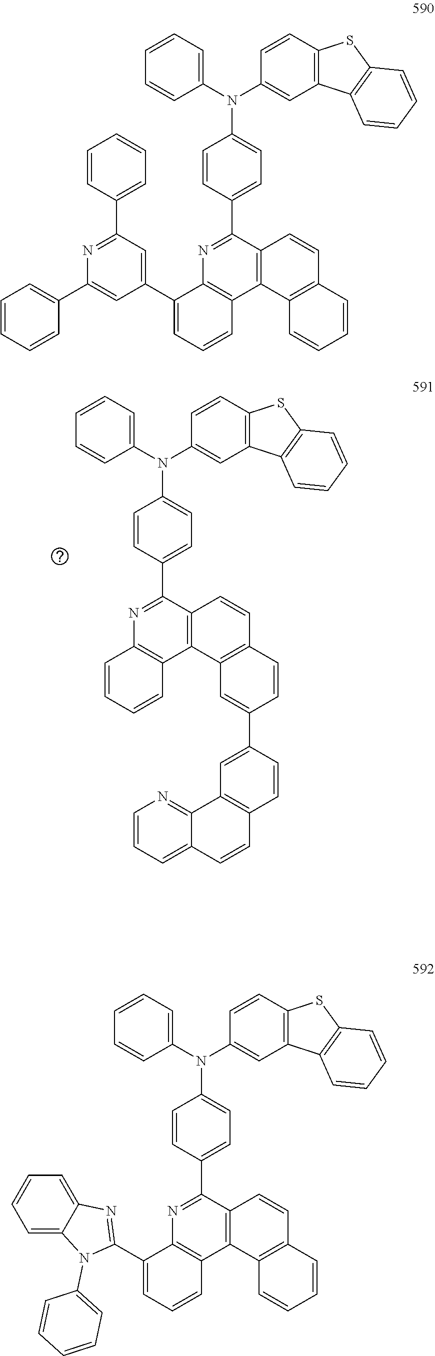 [SMILES: c1ccc(-c2cc(-c3cccc4c3nc(-c3ccc(N(c5ccccc5)c5ccc6sc7ccccc7c6c5)cc3)c3ccc5ccccc5c34)cc(-c3ccccc3)n2)cc1 ; c1ccc(N(c2ccc(-c3nc4ccccc4c4c3ccc3ccc(-c5ccc6ccc7cccnc7c6c5)cc34)cc2)c2ccc3sc4ccccc4c3c2)cc1 ; c1ccc(N(c2ccc(-c3nc4c(-c5nc6ccccc6n5-c5ccccc5)cccc4c4c3ccc3ccccc34)cc2)c2ccc3sc4ccccc4c3c2)cc1]

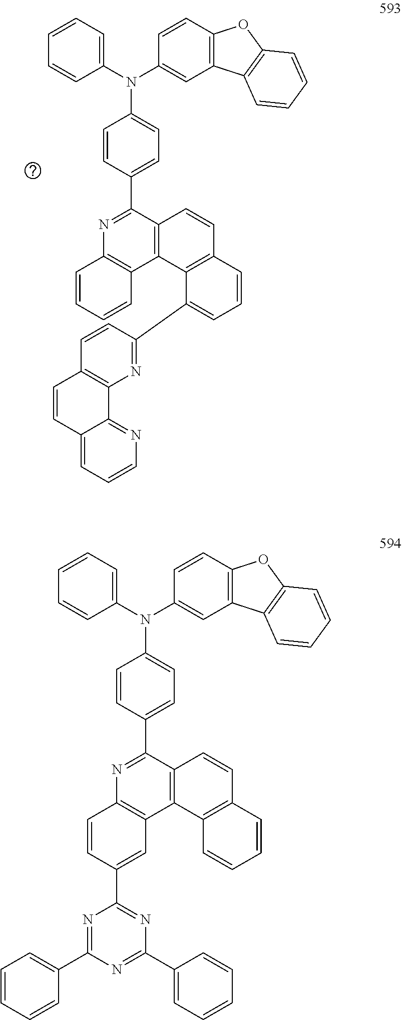 [SMILES: c1ccc(N(c2ccc(-c3nc4ccccc4c4c3ccc3cccc(-c5ccc6ccc7cccnc7c6n5)c34)cc2)c2ccc3oc4ccccc4c3c2)cc1 ; c1ccc(-c2nc(-c3ccccc3)nc(-c3ccc4nc(-c5ccc(N(c6ccccc6)c6ccc7oc8ccccc8c7c6)cc5)c5ccc6ccccc6c5c4c3)n2)cc1]

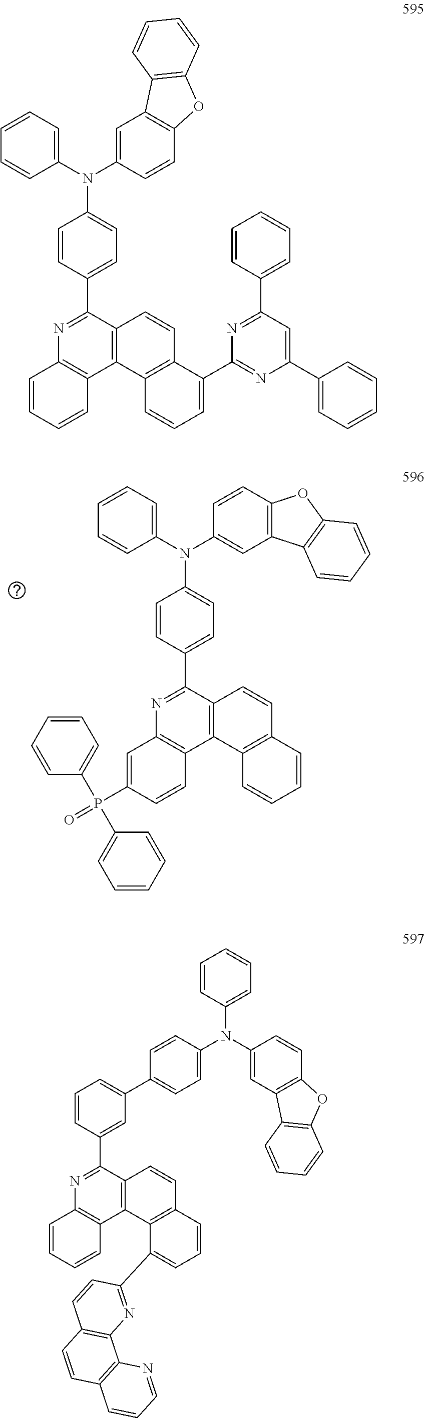 [SMILES: c1ccc(-c2cc(-c3ccccc3)nc(-c3cccc4c3ccc3c(-c5ccc(N(c6ccccc6)c6ccc7oc8ccccc8c7c6)cc5)nc5ccccc5c34)n2)cc1 ; O=P(c1ccccc1)(c1ccccc1)c1ccc2c(c1)nc(-c1ccc(N(c3ccccc3)c3ccc4oc5ccccc5c4c3)cc1)c1ccc3ccccc3c12 ; c1ccc(N(c2ccc(-c3cccc(-c4nc5ccccc5c5c4ccc4cccc(-c6ccc7ccc8cccnc8c7n6)c45)c3)cc2)c2ccc3oc4ccccc4c3c2)cc1]

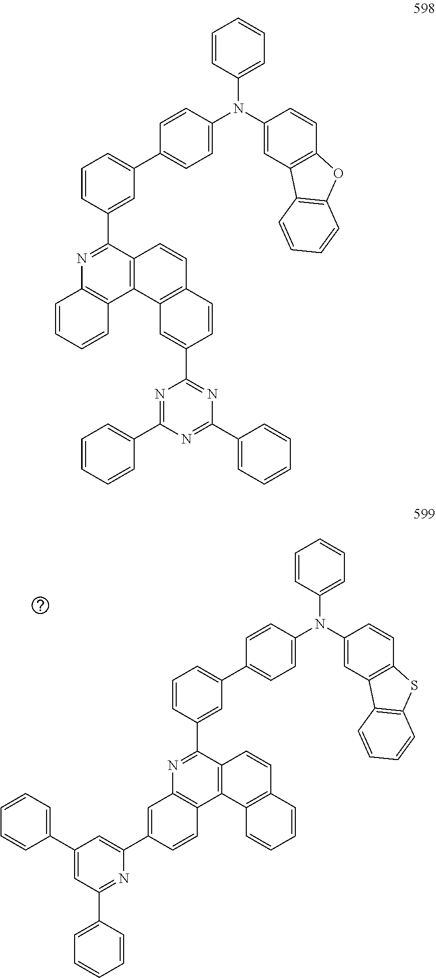 [SMILES: c1ccc(-c2nc(-c3ccccc3)nc(-c3ccc4ccc5c(-c6cccc(-c7ccc(N(c8ccccc8)c8ccc9oc%10ccccc%10c9c8)cc7)c6)nc6ccccc6c5c4c3)n2)cc1 ; c1ccc(-c2cc(-c3ccccc3)nc(-c3ccc4c(c3)nc(-c3cccc(-c5ccc(N(c6ccccc6)c6ccc7sc8ccccc8c7c6)cc5)c3)c3ccc5ccccc5c34)c2)cc1]

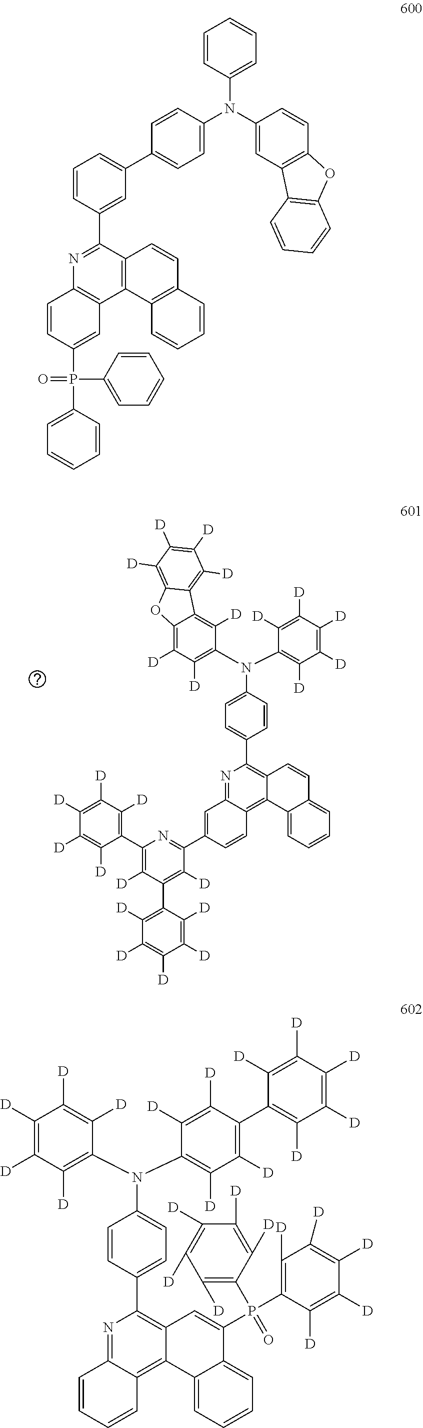

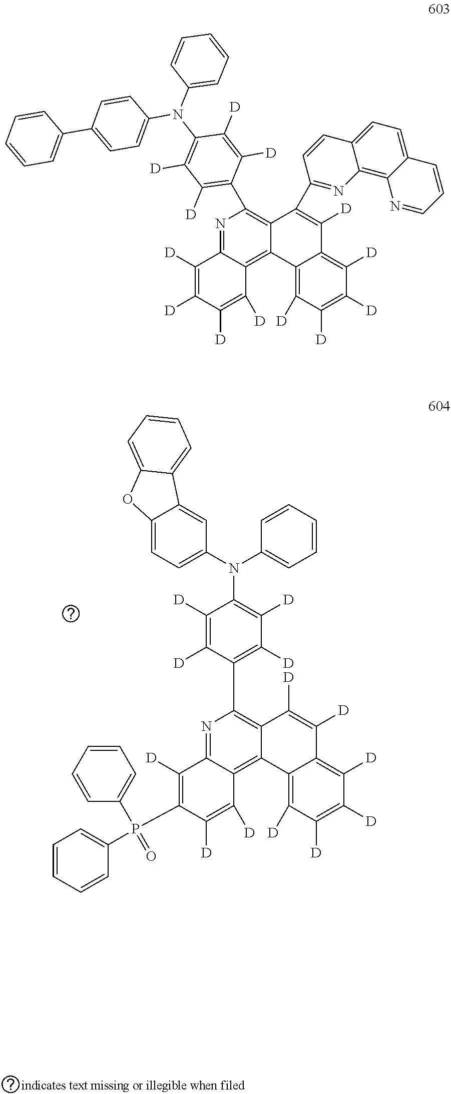 [SMILES: [2H]c1c([2H])c(N(c2ccccc2)c2ccc(-c3ccccc3)cc2)c([2H])c([2H])c1-c1nc2c([2H])c([2H])c([2H])c([2H])c2c2c1c(-c1ccc3ccc4cccnc4c3n1)c([2H])c1c([2H])c([2H])c([2H])c([2H])c12 ; [2H]c1c([2H])c(N(c2ccccc2)c2ccc3oc4ccccc4c3c2)c([2H])c([2H])c1-c1nc2c([2H])c(P(=O)(c3ccccc3)c3ccccc3)c([2H])c([2H])c2c2c1c([2H])c([2H])c1c([2H])c([2H])c([2H])c([2H])c12]

In an exemplary embodiment of the present application, the compound is just one example and is not limited thereto, and may include other compounds included in Chemical Formula 1 which includes an additional substituent. In addition, the substitution position of deuterium of the compound may be present while a specific position is excluded and hydrogen and deuterium are mixed during the process of deuterium substitution and synthesis.

Further, various substituents may be introduced into the structure of Chemical Formula 1 to synthesize a compound having inherent characteristics of a substituent introduced. For example, a substituent usually used for a hole injection material, a hole transport material, a light emitting material, an electron transport material and an electron injection material, which are used when manufacturing an organic light emitting device, may be introduced into the core structure to synthesize a material which satisfies conditions required for each organic material layer.

In addition, by introducing various substituents into the structure of Chemical Formula 1 or changing the binding position, the bandgap may be finely adjusted, and meanwhile, the characteristics at the interface between the organic material layers may be improved.

In addition, the compound of Chemical Formula 1 has excellent thermal stability, and such thermal stability provides driving stability to the organic light emitting device and improves service life characteristics.

In an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In another exemplary embodiment, provided is an organic light emitting device including: a first electrode; a second electrode provided to face the first electrode; and an organic material layer having one or more layers provided between the first electrode and the second electrode, in which one or more layers of the organic material layer include one heterocyclic compound represented by Chemical Formula 1.

In still another exemplary embodiment, provided is an organic light emitting device including: a first electrode; a second electrode provided to face the first electrode; and an organic material layer having one or more layers provided between the first electrode and the second electrode, in which one or more layers of the organic material layer include two or more of the heterocyclic compound represented by Chemical Formula 1.

In yet another exemplary embodiment, the heterocyclic compound represented by Chemical Formula 1 can be used as a light emitting material for a light emitting layer of the organic light emitting device.

In still yet another exemplary embodiment, the heterocyclic compound represented by Chemical Formula 1 can be used as a host material for a light emitting layer of the organic light emitting device.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device.

In an exemplary embodiment of the present application, the organic light emitting device may be a green organic light emitting device, and the heterocyclic compound represented by Chemical Formula 1 may be used as a material for the green organic light emitting device.

In an exemplary embodiment of the present application, the organic light emitting device may be a red organic light emitting device, and the heterocyclic compound represented by Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for a light emitting layer of the blue organic light emitting device.

In an exemplary embodiment of the present application, the organic light emitting device may be a green organic light emitting device, and the heterocyclic compound represented by Chemical Formula 1 may be used as a material for a light emitting layer of the green organic light emitting device.

In an exemplary embodiment of the present application, the organic light emitting device may be a red organic light emitting device, and the heterocyclic compound represented by Chemical Formula 1 may be used as a material for a light emitting layer of the red organic light emitting device.

In an exemplary embodiment of the present application, the specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃, which is a green phosphorescent dopant, may be used.

In an exemplary embodiment of the present application, as the iridium-based dopant, (piq)₂(Ir)(acac), which is a red phosphorescent dopant, may be used.

In an exemplary embodiment of the present application, provided is an organic light emitting device, in which the organic material layer of the organic light emitting device includes a light emitting layer, and the light emitting layer includes the heterocyclic compound.

In an exemplary embodiment of the present application, provided is an organic light emitting device, in which the organic material layer of the organic light emitting device includes a light emitting layer, and the light emitting layer includes a host material, and the host material includes the heterocyclic compound.

In the organic light emitting device of the present invention, the organic material layer includes an electron injection layer or an electron transport layer, and the electron injection layer or electron transport layer may include the heterocyclic compound.

In another organic light emitting device, the organic material layer includes a hole blocking layer, and the hole blocking layer may include the heterocyclic compound.

In still another organic light emitting device, the organic material layer includes an electron blocking layer, and the electron blocking layer may include the heterocyclic compound.

In yet another organic light emitting device, the organic material layer includes a hole transport layer, a light emitting layer or an electron blocking layer, and the hole transport layer, the light emitting layer or the electron blocking layer may include the heterocyclic compound.

In still yet another organic light emitting device, the organic material layer includes a hole transport layer or a hole transport auxiliary layer, and the hole transport layer or the hole transport auxiliary layer may include the heterocyclic compound.

In a further organic light emitting device, the organic material layer includes a light emitting auxiliary layer or an N-type charge generation layer (N-CGL), and the light emitting auxiliary layer or the N-type charge generation layer (N-CGL) may include the heterocyclic compound.

In the organic light emitting device of the present application, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

The organic light emitting device of the present invention may further include one or two or more layers selected from the group consisting of a light emitting layer, a hole injection layer, a hole transport layer, an electron injection layer, an electron transport layer, an electron blocking layer, and a hole blocking layer.

The organic light emitting device of the present invention may further include one or two or more layers selected from the group consisting of a light emitting layer, a hole injection layer, a hole transport layer, an electron blocking layer, a hole blocking layer, a charge generation layer, an electron transport layer and an electron injection layer.

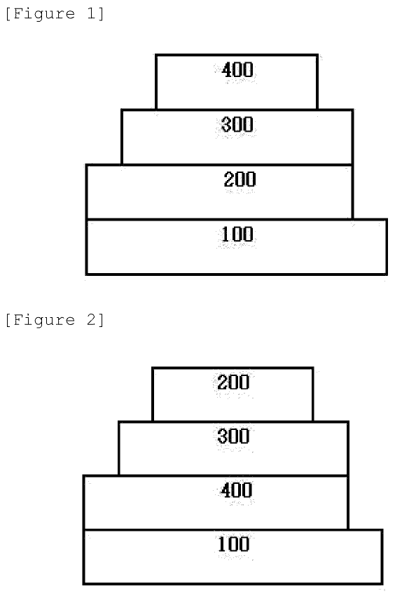

FIGS. 1 to 4 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

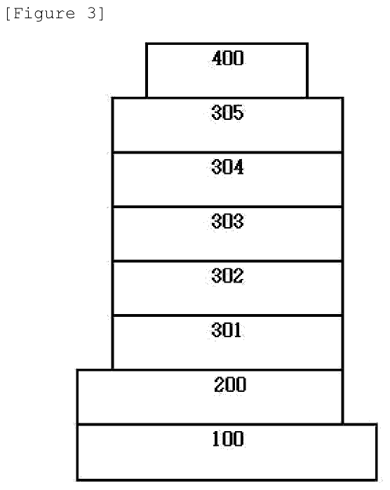

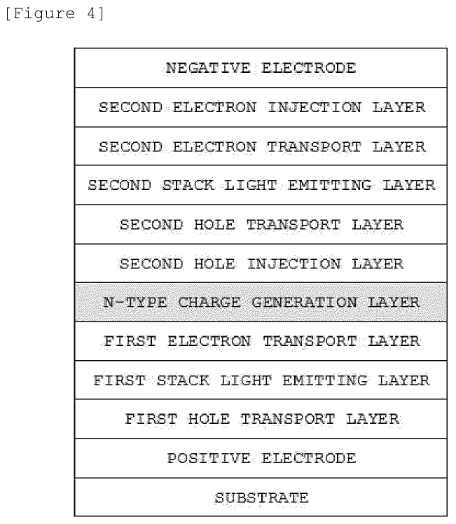

FIGS. 3 and 4 exemplify a case where an organic material layer is a multilayer.

An organic light emitting device according to FIG. 3 includes a hole injection layer 301, a hole transport layer 302, a light emitting layer 303, an electron transport layer 304, and an electron injection layer 305. However, the scope of the present application is not limited by the stacking structure as described above, and if necessary, the other layers except for the light emitting layer may be omitted, and another necessary functional layer may be further added.

An organic material layer including Chemical Formula 1 may additionally include another material, if necessary.

Further, the organic light emitting device according to an exemplary embodiment of the present application includes a first electrode, a second electrode, and two or more stacks provided between the first electrode and the second electrode, the two or more stacks each independently include a light emitting layer, a charge generation layer is included between the two or more stacks, and the charge generation layer includes the heterocyclic compound represented by Chemical Formula 1.

In addition, the organic light emitting device according to an exemplary embodiment of the present application includes a first electrode, a first stack provided on the first electrode and including a first light emitting layer, a charge generation layer provided on the first stack, a second stack provided on the charge generation layer and including a second light emitting layer, and a second electrode provided on the second stack. In this case, the charge generation layer may include the heterocyclic compound represented by Chemical Formula 1. Furthermore, the first stack and the second stack may each independently further include one or more of the above-described hole injection layer, hole transport layer, hole blocking layer, electron transport layer, electron injection layer, and the like.

The charge generation layer may be an N-type charge generation layer, and the charge generation layer may additionally include a dopant known in the art in addition to the heterocyclic compound represented by Chemical Formula 1.

As the organic light emitting device according to an exemplary embodiment of the present application, an organic light emitting device having a 2-stack tandem structure is schematically illustrated in FIG. 4.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

Hereinafter, the present specification will be described in more detail through Examples, but these Examples are provided only for exemplifying the present application, and are not intended to limit the scope of the present application.

### SYNTHESIS EXAMPLES

### [Preparation Example 1] Preparation of Compound 001

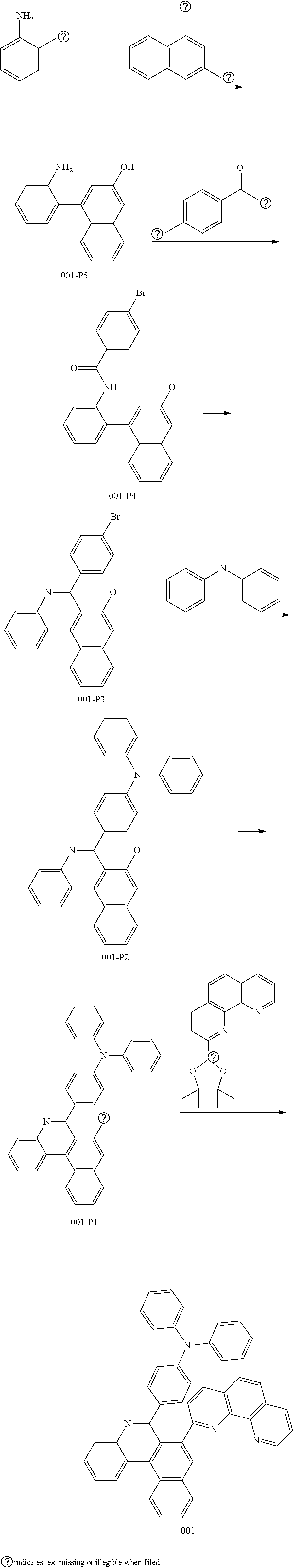

1) Preparation of Compound 001-P5

After a compound (2-aminophenyl)boronic acid (15 g, 109.54 mmol) and 4-bromonaphthalen-2-ol (26.88 g, 120.49 mmol) were dissolved in 150 ml of 1,4-dioxane and 45 ml of distilled water, Pd(PPh₃)₄ (3.84 g, 5.48 mmol) and K₂CO₃ (29.02 g, 273.84 mmol) were added thereto, and then the resulting mixture was refluxed for 12 hours. After the reaction was completed, the reaction product was cooled to room temperature, and then extracted with distilled water and ethyl acetate (EA). After the organic layer was dried over anhydrous MgSO₄, the solvent was removed by a rotary evaporator, and then the residue was purified by column chromatography using dichloromethane and hexane as an eluting solvent, thereby obtaining 20.1 g (78%) of Target Compound 001-P5.

2) Preparation of Compound 001-P4

After Compound 001-P5 (20.1 g, 85.43 mmol) was dissolved in 300 ml of dichloromethane, 4-bromobenzoyl chloride (20.62 g, 93.97 mmol) and trimethylamine (TEA) (8.64 g, 85.43 mmol) were added thereto at 0° C., and then the resulting mixture was stirred at room temperature for 2 hours. After the reaction was completed, EA and distilled water were added to a reaction vessel to solidify the resulting product, and then the resulting solid was collected to obtain Target Compound 001-P4 (28.2 g, 79%).

3) Preparation of Compound 001-P3

After Compound 001-P4 (28.2 g, 67.42 mmol) was dissolved in 300 ml of nitrobenzene, POCl₃ (15.51 g, 101.13 mmol) was added thereto, and then the resulting mixture was stirred at 150° C. for 18 hours. After the reaction was completed, nitrobenzene was removed by distillation under reduced pressure, and then the residue was cooled to room temperature, and extracted with distilled water and EA. After the organic layer was dried over anhydrous MgSO₄, the solvent was removed by a rotary evaporator, and then the residue was purified by column chromatography using dichloromethane and hexane as an eluting solvent, thereby obtaining 19.2 g (71%) of Target Compound 001-P3.

4) Preparation of Compound 001-P2

After Compound 001-P3 (19.2 g, 47.97 mmol) and diphenylamine (9.74 g, 57.56 mmol) were dissolved in 200 ml of toluene, Pd₂(dba)₃ (2.2 g, 2.4 mmol), xphos (2.29 g, 4.8 mmol), and NaOtBu (34.3 g, 71.95 mmol) were added thereto and the resulting mixture was stirred under reflux for 3 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 001-P2 (17.5 g, 74%).

5) Preparation of Compound 001-P1

After Compound 001-P2 (17.5 g, 35.82 mmol) was dissolved in dichloromethane, TEA (5.94 g, 42.98 mmol) was added thereto, and then triflic anhydride (7.26 ml, 42.98 mmol) was added dropwise thereto at 0° C. Thereafter, the resulting mixture was stirred at room temperature for 5 hours. After the reaction was completed, the resulting product was extracted with dichloromethane and distilled water, then the solvent was removed from the filtrate by a rotary evaporator, and then the residue was purified by column chromatography using dichloromethane and hexane as an eluting solvent, thereby obtaining 12.9 g (58%) of Target Compound 001-P1.

6) Preparation of Compound 001

After Compound 001-P1 (12.9 g, 20.78 mmol) and 2-(4,4,5,5-tetramethyl-1,3,2-dioxaborolan-2-yl)-1,10-phenanthroline (7 g, 22.86 mmol) were dissolved in 150 ml of 1,4-dioxane and 40 ml of distilled water, Pd(PPh₃)₄ (1.2 g, 1.04 mmol) and K₂CO₃ (5.51 g, 51.96 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 001 (8.8 g, 65%).

A compound was synthesized in the same manner as in Preparation Example 1 using Intermediate A in the following Table 1 instead of (2-aminophenyl)boronic acid (A), Intermediate B in the following Table 1 instead of 4-bromonaphthalen-2-ol (B), Intermediate C instead of 4-bromobenzoyl chloride (C), Intermediate D instead of diphenylamine (D), and Intermediate E instead of 2-(4,4,5,5-tetramethyl-1,3,2-dioxaborolan-2-yl)-1,10-phenanthroline (E).

TABLE 1

| COM- | | | | | | |
|---|---|---|---|---|---|---|
| POUND | | | | | | |
| NO. | INTERMEDIATE A | INTERMEDIATE B | INTERMEDIATE C | INTERMEDIATE D | INTERMEDIATE E | YIELD |
| 017 | 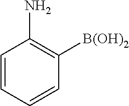 | 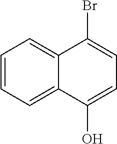 [SMILES: Oc1ccc(Br)c2ccccc12] | 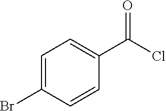 | 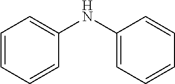 [SMILES: c1ccc(Nc2ccccc2)cc1] | 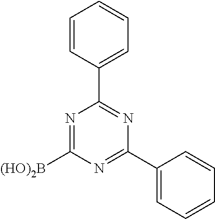 [SMILES: OB(O)c1nc(-c2ccccc2)nc(-c2ccccc2)n1] | 56% |
| 025 | 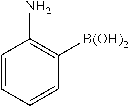 | 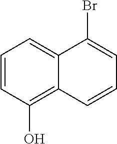 [SMILES: Oc1cccc2c(Br)cccc12] | 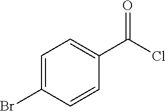 | 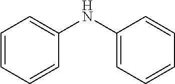 [SMILES: c1ccc(Nc2ccccc2)cc1] | 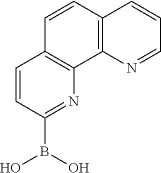 [SMILES: OB(O)c1ccc2ccc3cccnc3c2n1] | 52% |
| 041 | 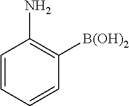 | 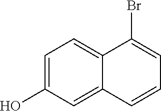 [SMILES: Oc1ccc2c(Br)cccc2c1] | 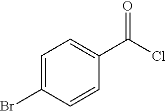 | 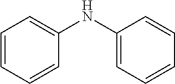 [SMILES: c1ccc(Nc2ccccc2)cc1] | 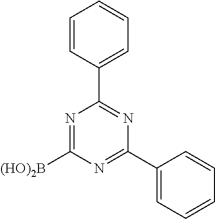 [SMILES: OB(O)c1nc(-c2ccccc2)nc(-c2ccccc2)n1] | 50% |
| 047 | 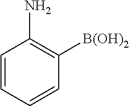 | 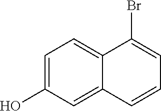 [SMILES: Oc1ccc2c(Br)cccc2c1] | 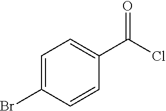 | 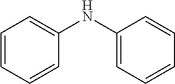 [SMILES: c1ccc(Nc2ccccc2)cc1] | 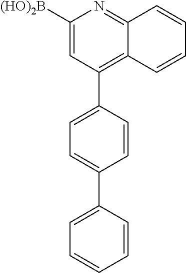 [SMILES: OB(O)c1cc(-c2ccc(-c3ccccc3)cc2)c2ccccc2n1] | 51% |
| 054 | 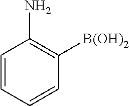 | 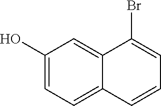 [SMILES: Oc1ccc2cccc(Br)c2c1] | 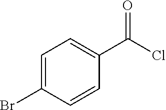 | 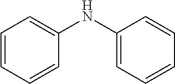 [SMILES: c1ccc(Nc2ccccc2)cc1] | 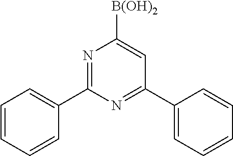 [SMILES: OB(O)c1cc(-c2ccccc2)nc(-c2ccccc2)n1] | 52% |
| 072 | 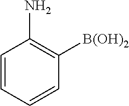 | 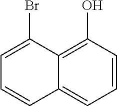 [SMILES: Oc1cccc2cccc(Br)c12] | 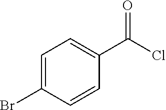 | 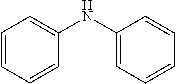 [SMILES: c1ccc(Nc2ccccc2)cc1] | 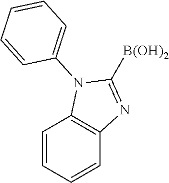 [SMILES: OB(O)c1nc2ccccc2n1-c1ccccc1] | 48% |
| 079 | 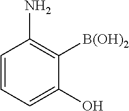 [SMILES: Nc1cccc(O)c1B(O)O] | 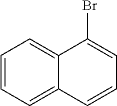 [SMILES: Brc1cccc2ccccc12] | 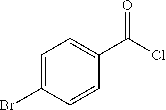 | 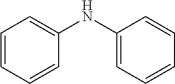 [SMILES: c1ccc(Nc2ccccc2)cc1] | 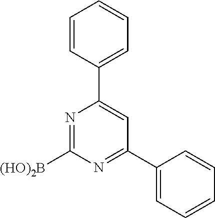 [SMILES: OB(O)c1nc(-c2ccccc2)cc(-c2ccccc2)n1] | 51% |
| 092 | 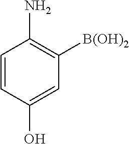 [SMILES: Nc1ccc(O)cc1B(O)O] | 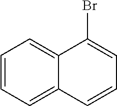 [SMILES: Brc1cccc2ccccc12] | 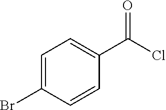 | 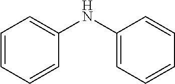 [SMILES: c1ccc(Nc2ccccc2)cc1] | 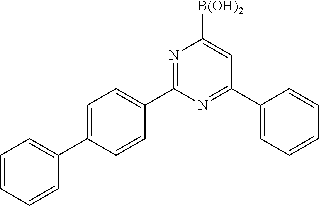 [SMILES: OB(O)c1cc(-c2ccccc2)nc(-c2ccc(-c3ccccc3)cc2)n1] | 53% |
| 102 | 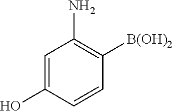 [SMILES: Nc1cc(O)ccc1B(O)O] | 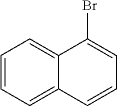 [SMILES: Brc1cccc2ccccc12] | 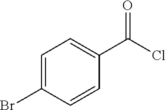 | 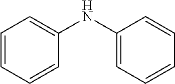 [SMILES: c1ccc(Nc2ccccc2)cc1] | 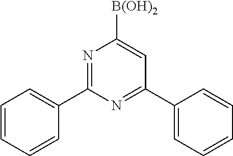 [SMILES: OB(O)c1cc(-c2ccccc2)nc(-c2ccccc2)n1] | 49% |
| 115 | 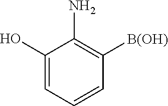 | 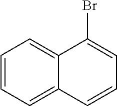 [SMILES: Brc1cccc2ccccc12] | 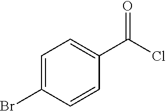 | 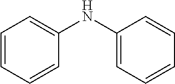 [SMILES: c1ccc(Nc2ccccc2)cc1] | 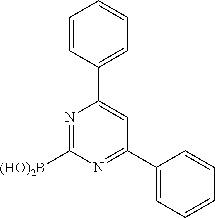 [SMILES: OB(O)c1nc(-c2ccccc2)cc(-c2ccccc2)n1] | 58% |
| 137 | 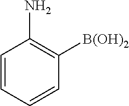 | 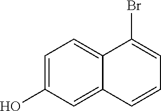 [SMILES: Oc1ccc2c(Br)cccc2c1] | 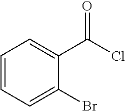 | 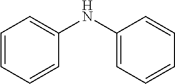 [SMILES: c1ccc(Nc2ccccc2)cc1] | 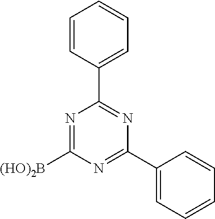 [SMILES: OB(O)c1nc(-c2ccccc2)nc(-c2ccccc2)n1] | 59% |
| 154 | 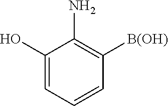 | 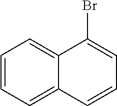 [SMILES: Brc1cccc2ccccc12] | 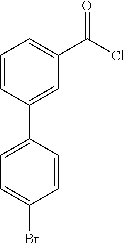 [SMILES: O=C(Cl)c1cccc(-c2ccc(Br)cc2)c1] | 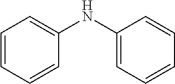 [SMILES: c1ccc(Nc2ccccc2)cc1] | 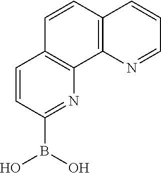 [SMILES: OB(O)c1ccc2ccc3cccnc3c2n1] | 54% |
| 569 | 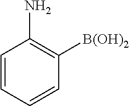 | 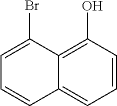 [SMILES: Oc1cccc2cccc(Br)c12] | 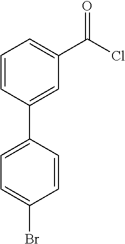 [SMILES: O=C(Cl)c1cccc(-c2ccc(Br)cc2)c1] | 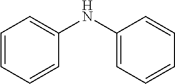 [SMILES: c1ccc(Nc2ccccc2)cc1] | 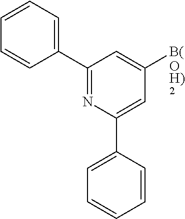 [SMILES: OB(O)c1cc(-c2ccccc2)nc(-c2ccccc2)c1] | 53% |
| 571 | 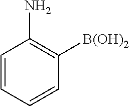 | 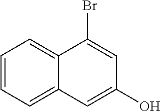 [SMILES: Oc1cc(Br)c2ccccc2c1] | 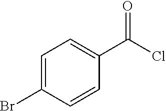 | 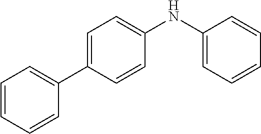 [SMILES: c1ccc(Nc2ccc(-c3ccccc3)cc2)cc1] | 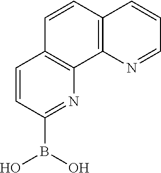 [SMILES: OB(O)c1ccc2ccc3cccnc3c2n1] | 55% |
| 576 | 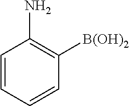 | 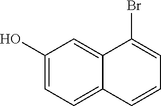 [SMILES: Oc1ccc2cccc(Br)c2c1] | 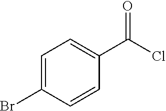 | 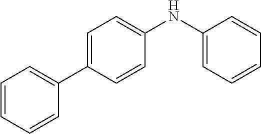 [SMILES: c1ccc(Nc2ccc(-c3ccccc3)cc2)cc1] | 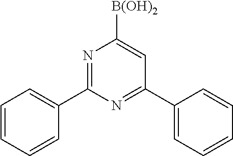 [SMILES: OB(O)c1cc(-c2ccccc2)nc(-c2ccccc2)n1] | 49% |
| 586 | 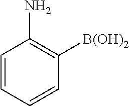 | 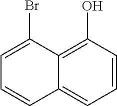 [SMILES: Oc1cccc2cccc(Br)c12] | 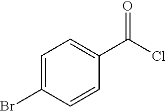 | 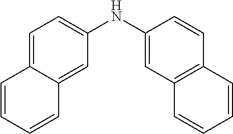 [SMILES: c1ccc2cc(Nc3ccc4ccccc4c3)ccc2c1] | 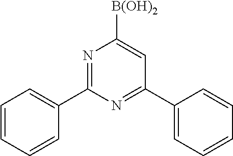 [SMILES: OB(O)c1cc(-c2ccccc2)nc(-c2ccccc2)n1] | 52% |
| 591 | 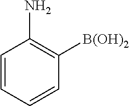 | 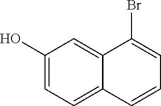 [SMILES: Oc1ccc2cccc(Br)c2c1] | 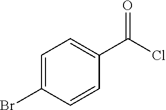 | 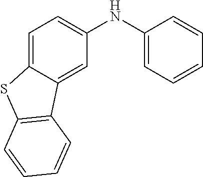 [SMILES: c1ccc(Nc2ccc3sc4ccccc4c3c2)cc1] | 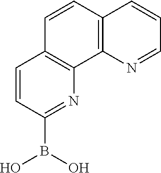 [SMILES: OB(O)c1ccc2ccc3cccnc3c2n1] | 57% |
| 598 | 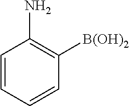 | 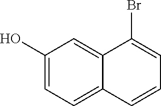 [SMILES: Oc1ccc2cccc(Br)c2c1] | 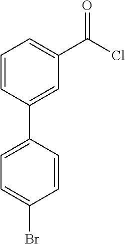 [SMILES: O=C(Cl)c1cccc(-c2ccc(Br)cc2)c1] | 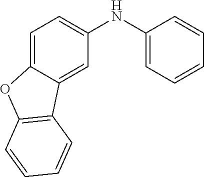 [SMILES: c1ccc(Nc2ccc3oc4ccccc4c3c2)cc1] | 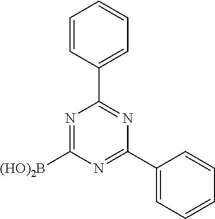 [SMILES: OB(O)c1nc(-c2ccccc2)nc(-c2ccccc2)n1] | 53% |
| 599 | 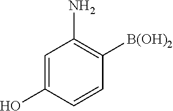 [SMILES: Nc1cc(O)ccc1B(O)O] | 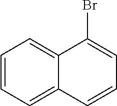 [SMILES: Brc1cccc2ccccc12] | 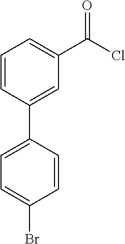 [SMILES: O=C(Cl)c1cccc(-c2ccc(Br)cc2)c1] | 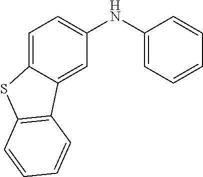 [SMILES: c1ccc(Nc2ccc3sc4ccccc4c3c2)cc1] | 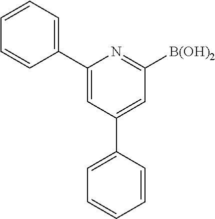 [SMILES: OB(O)c1cc(-c2ccccc2)cc(-c2ccccc2)n1] | 55% |

### [Preparation Example 2] Preparation of Compound 003

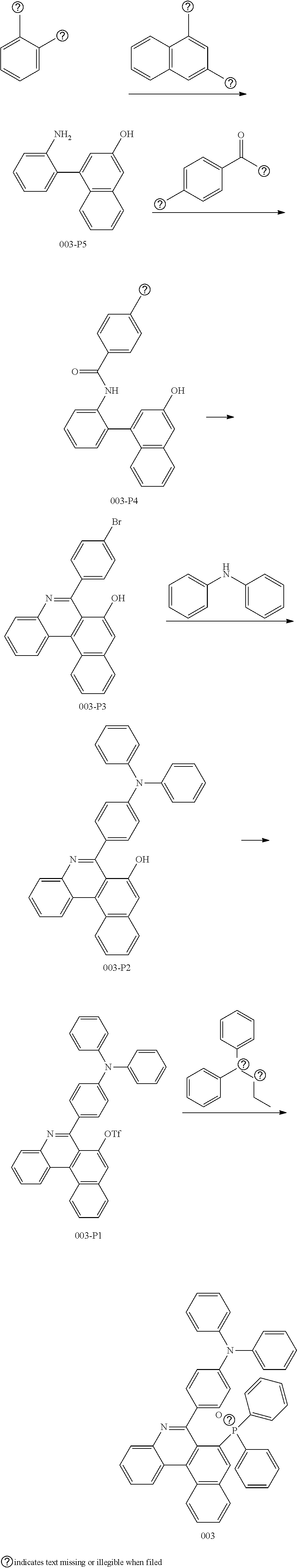

Synthesis was performed in the same manner as in Preparation Example 1 except for the synthesis of Compound 003 in Preparation Example 2.

1) Preparation of Compound 003

After Compound 001-P1 (12.9 g, 20.78 mmol), ethoxydiphenylphosphane (14.5 g, 62.35 mmol) and NiCl 2 (1.35 g, 10.39 mmol) were dissolved in 150 ml of dimethylacetamide, the resulting solution was stirred under reflux for 12 hours. After the reaction was completed, the solvent was removed from the reaction solution by a rotary evaporator, and then the resulting product was purified by column chromatography using EA and hexane as an eluting solvent, thereby obtaining 8.1 g (58%) of Compound 003.

A compound was synthesized in the same manner as in Preparation Example 2 using Intermediate A in the following Table 2 instead of (2-aminophenyl)boronic acid (A), Intermediate B instead of 4-bromonaphthalen-2-ol (B), Intermediate C instead of 4-bromobenzoyl chloride (C), and Intermediate D instead of diphenylamine (D).

TABLE 2

| COM- | | | |
|---|---|---|---|
| POUND | | | |
| NO. | INTERMEDIATE A | INTERMEDIATE B | INTERMEDIATE C |
| 027 | 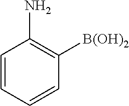 | 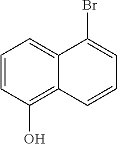 [SMILES: Oc1cccc2c(Br)cccc12] | 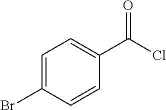 |
| 123 | 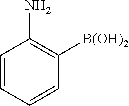 | 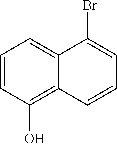 [SMILES: Oc1cccc2c(Br)cccc12] | 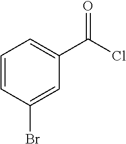 |
| 146 | 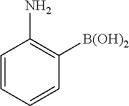 | 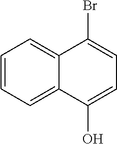 [SMILES: Oc1ccc(Br)c2ccccc12] | 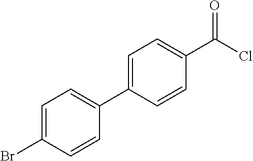 [SMILES: O=C(Cl)c1ccc(-c2ccc(Br)cc2)cc1] |
| 583 | 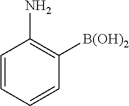 | 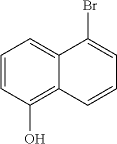 [SMILES: Oc1cccc2c(Br)cccc12] | 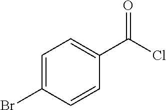 |
| 596 | 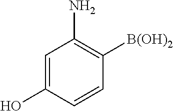 [SMILES: Nc1cc(O)ccc1B(O)O] | 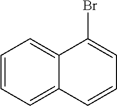 [SMILES: Brc1cccc2ccccc12] | 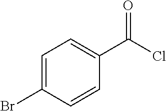 |
| | COM- | | |
| | POUND | | |
| | NO. | INTERMEDIATE D | YIELD |
| | 027 | 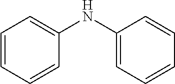 [SMILES: c1ccc(Nc2ccccc2)cc1] | 54% |
| | 123 | 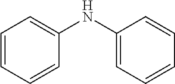 [SMILES: c1ccc(Nc2ccccc2)cc1] | 55% |
| | 146 | 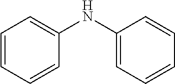 [SMILES: c1ccc(Nc2ccccc2)cc1] | 52% |
| | 583 | 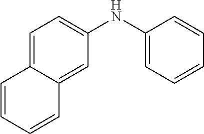 [SMILES: c1ccc(Nc2ccc3ccccc3c2)cc1] | 57% |
| | 596 | 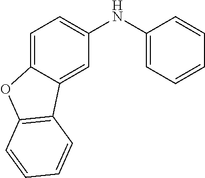 [SMILES: c1ccc(Nc2ccc3oc4ccccc4c3c2)cc1] | 54% |

### [Preparation Example 3] Preparation of Compound 161

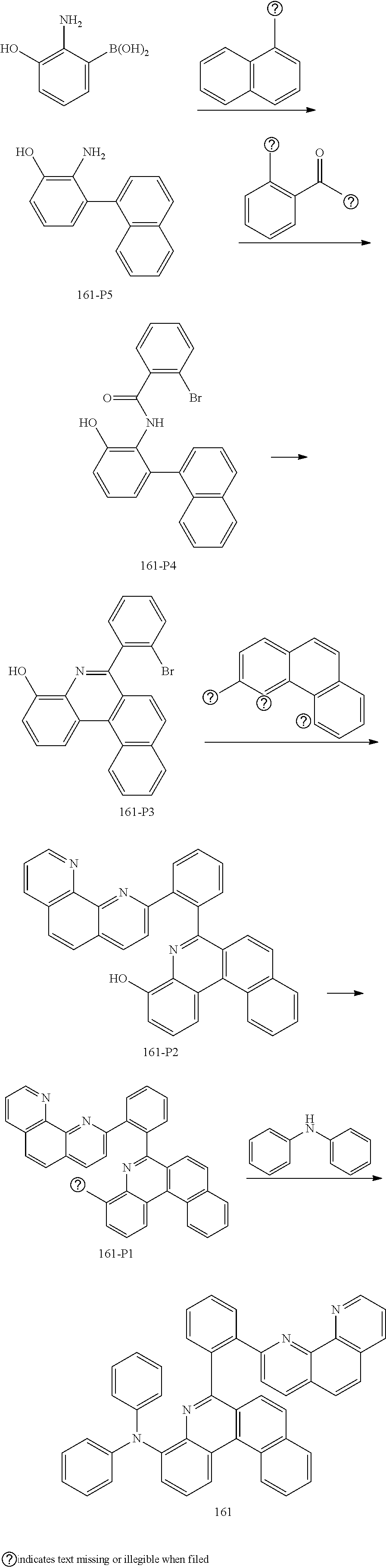

1) Preparation of Compound 161-P5

After a compound (2-amino-3-hydroxyphenyl)boronic acid (15 g, 98.08 mmol) and 1-bromonaphthalene (22.57 g, 107.89 mmol) were dissolved in 150 ml of 1,4-dioxane and 45 ml of distilled water, Pd(PPh₃)₄ (5.67 g, 4.9 mmol) and K₂CO₃ (25.99 g, 245.19 mmol) were added thereto, and then the resulting mixture was refluxed for 12 hours. After the reaction was completed, the resulting product was cooled to normal temperature, and then extraction was performed with distilled water and EA. After the organic layer was dried over anhydrous MgSO₄, the solvent was removed by a rotary evaporator, and then the residue was purified by column chromatography using dichloromethane and hexane as an eluting solvent, thereby obtaining 18.4 g (80%) of Target Compound 161-P5.

2) Preparation of Compound 161-P4

After Compound 161-P5 (18.4 g, 78.2 mmol) was dissolved in 300 ml of dichloromethane, 2-bromobenzoyl chloride (18.88 g, 86.03 mmol) and trimethylamine (TEA) (10.81 g, 78.2 mmol) were added thereto at 0° C., and then the resulting mixture was stirred at room temperature for 2 hours. After the reaction was completed, EA and distilled water were added to a reaction vessel to solidify the resulting product, and then the resulting solid was collected to obtain Target Compound 161-P4 (24.5 g, 75%).

3) Preparation of Compound 161-P3

After Compound 161-P4 (24.5 g, 58.57 mmol) was dissolved in 300 ml of nitrobenzene, POCl₃ (13.47 g, 87.86 mmol) was added thereto, and then the resulting mixture was stirred at 150° C. for 18 hours. After the reaction was completed, nitrobenzene was removed by distillation under reduced pressure, and then the residue was cooled to room temperature, and extracted with distilled water and EA. After the organic layer was dried over anhydrous MgSO₄, the solvent was removed by a rotary evaporator, and then the residue was purified by column chromatography using dichloromethane and hexane as an eluting solvent, thereby obtaining 19.2 g (82%) of Target Compound 161-P3.

4) Preparation of Compound 161-P2

After Compound 161-P3 (19.2 g, 47.97 mol) and 1,10-phenanthrolin-2-ylboronic acid (11.82 g, 52.76 mmol) were 200 ml of 1,4-dioxane and 60 ml of distilled water, Pd(PPh₃)₄ (2.77 g, 2.4 mmol) and K₂CO₃ (12.71 g, 119.92 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 161-P2 (14.5 g, 60%).

5) Preparation of Compound 161-P1

After Compound 161-P2 (14.5 g, 29.03 mmol) was dissolved in dichloromethane, TEA (4.81 g, 34.83 mmol) was added thereto, and then triflic anhydride (7.36 ml, 43.54 mmol) was added dropwise thereto at 0° C. Thereafter, the resulting mixture was stirred at room temperature for 5 hours. After the reaction was completed, the resulting product was extracted with dichloromethane and distilled water, then the solvent was removed from the filtrate by a rotary evaporator, and then the residue was purified by column chromatography using dichloromethane and hexane as an eluting solvent, thereby obtaining 12.8 g (70%) of Target Compound 161-P1.

6) Preparation of Compound 161

After Compound 161-P1 (12.8 g, 20.27 mmol) and diphenylamine (3.77 g, 22.29 mmol) were dissolved in 200 ml of toluene, Pd₂(dba)₃ (0.93 g, 1.01 mmol), xphos (0.97 g, 2.03 mmol), and NaOtBu (3.9 g, 40.53 mmol) were added thereto and the resulting mixture was stirred under reflux for 3 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 161 (8.7 g, 66%).

A compound was synthesized in the same manner as in Preparation Example 3 using Intermediate A in the following Table 3 instead of (2-amino-3-hydroxyphenyl)boronic acid (A), Intermediate B in the following Table 3 instead of 1-bromonaphthalene (B), Intermediate C instead of 2-bromobenzoyl chloride (C), and Intermediate D instead of 1,10-phenanthrolin-2-ylboronic acid (D).

TABLE 3

| COMPOUND | | | | | |
|---|---|---|---|---|---|
| NO. | INTERMEDIATE A | INTERMEDIATE B | INTERMEDIATE C | INTERMEDIATE D | YIELD |
| 192 | 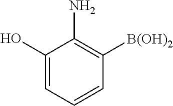 [SMILES: Nc1c(O)cccc1B(O)O] | 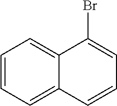 [SMILES: Brc1cccc2ccccc12] | 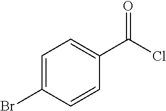 | 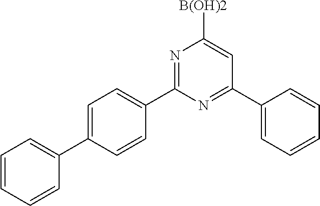 [SMILES: OB(O)c1cc(-c2ccccc2)nc(-c2ccc(-c3ccccc3)cc2)n1] | 54% |
| 194 | 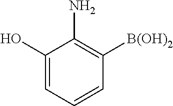 [SMILES: Nc1c(O)cccc1B(O)O] | 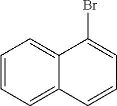 [SMILES: Brc1cccc2ccccc12] | 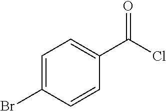 | 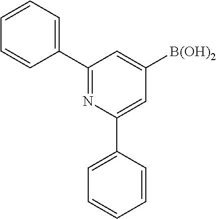 [SMILES: OB(O)c1cc(-c2ccccc2)nc(-c2ccccc2)c1] | 55% |
| 207 | 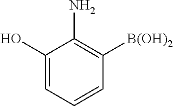 [SMILES: Nc1c(O)cccc1B(O)O] | 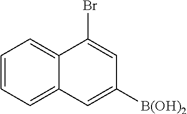 [SMILES: OB(O)c1cc(Br)c2ccccc2c1] | 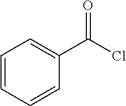 | 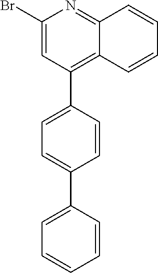 [SMILES: Brc1cc(-c2ccc(-c3ccccc3)cc2)c2ccccc2n1] | 54% |
| 218 | 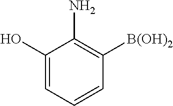 [SMILES: Nc1c(O)cccc1B(O)O] | 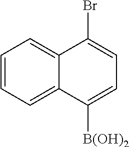 [SMILES: OB(O)c1ccc(Br)c2ccccc12] | 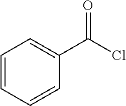 | 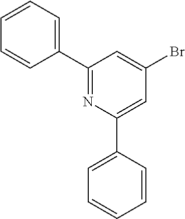 [SMILES: Brc1cc(-c2ccccc2)nc(-c2ccccc2)c1] | 55% |
| 226 | 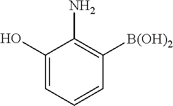 [SMILES: Nc1c(O)cccc1B(O)O] | 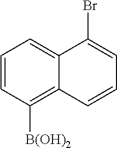 [SMILES: OB(O)c1cccc2c(Br)cccc12] | 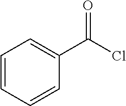 | 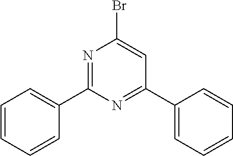 [SMILES: Brc1cc(-c2ccccc2)nc(-c2ccccc2)n1] | 55% |
| 239 | 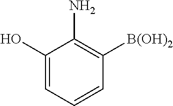 [SMILES: Nc1c(O)cccc1B(O)O] | 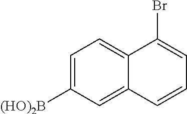 [SMILES: OB(O)c1ccc2c(Br)cccc2c1] | 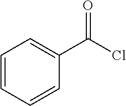 | 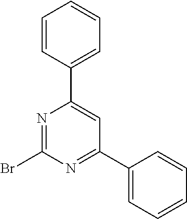 [SMILES: Brc1nc(-c2ccccc2)cc(-c2ccccc2)n1] | 59% |
| 245 | 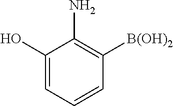 [SMILES: Nc1c(O)cccc1B(O)O] | 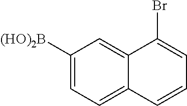 [SMILES: OB(O)c1ccc2cccc(Br)c2c1] | 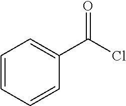 | 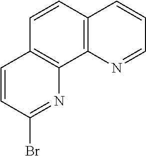 [SMILES: Brc1ccc2ccc3cccnc3c2n1] | 62% |
| 253 | 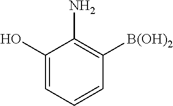 [SMILES: Nc1c(O)cccc1B(O)O] | 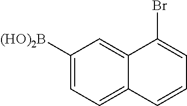 [SMILES: OB(O)c1ccc2cccc(Br)c2c1] | 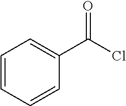 | 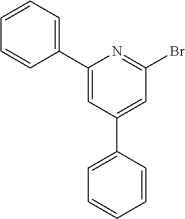 [SMILES: Brc1cc(-c2ccccc2)cc(-c2ccccc2)n1] | 55% |
| 268 | 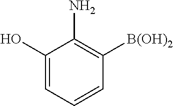 [SMILES: Nc1c(O)cccc1B(O)O] | 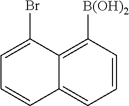 [SMILES: OB(O)c1cccc2cccc(Br)c12] | 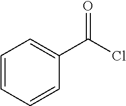 | 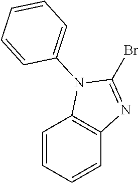 [SMILES: Brc1nc2ccccc2n1-c1ccccc1] | 52% |
| 274 | 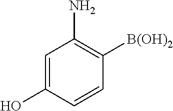 [SMILES: Nc1cc(O)ccc1B(O)O] | 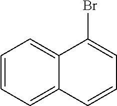 [SMILES: Brc1cccc2ccccc12] | 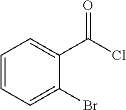 | 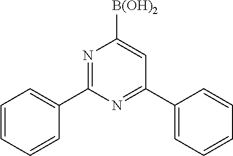 [SMILES: OB(O)c1cc(-c2ccccc2)nc(-c2ccccc2)n1] | 52% |
| 278 | 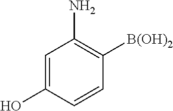 [SMILES: Nc1cc(O)ccc1B(O)O] | 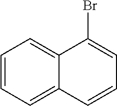 [SMILES: Brc1cccc2ccccc12] | 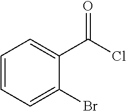 | 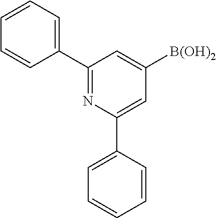 [SMILES: OB(O)c1cc(-c2ccccc2)nc(-c2ccccc2)c1] | 59% |
| 281 | 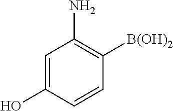 [SMILES: Nc1cc(O)ccc1B(O)O] | 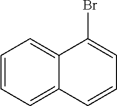 [SMILES: Brc1cccc2ccccc12] | 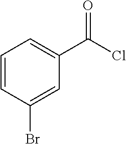 | 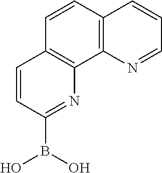 [SMILES: OB(O)c1ccc2ccc3cccnc3c2n1] | 51% |
| 300 | 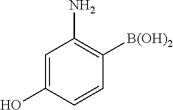 [SMILES: Nc1cc(O)ccc1B(O)O] | 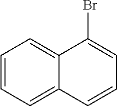 [SMILES: Brc1cccc2ccccc12] | 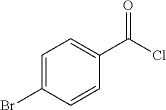 | 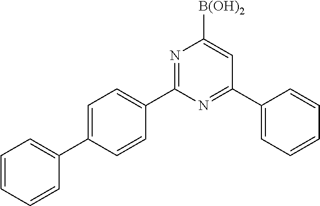 [SMILES: OB(O)c1cc(-c2ccccc2)nc(-c2ccc(-c3ccccc3)cc2)n1] | 63% |
| 302 | 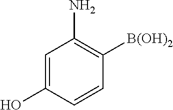 [SMILES: Nc1cc(O)ccc1B(O)O] | 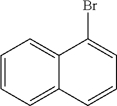 [SMILES: Brc1cccc2ccccc12] | 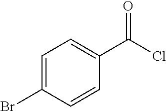 | 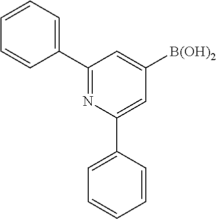 [SMILES: OB(O)c1cc(-c2ccccc2)nc(-c2ccccc2)c1] | 61% |
| 314 | 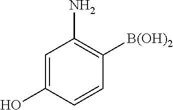 [SMILES: Nc1cc(O)ccc1B(O)O] | 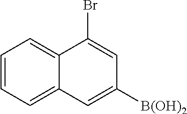 [SMILES: OB(O)c1cc(Br)c2ccccc2c1] | 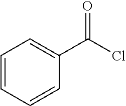 | 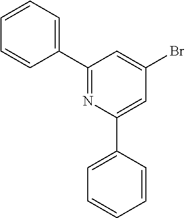 [SMILES: Brc1cc(-c2ccccc2)nc(-c2ccccc2)c1] | 52% |
| 324 | 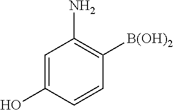 [SMILES: Nc1cc(O)ccc1B(O)O] | 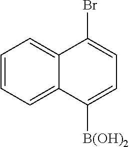 [SMILES: OB(O)c1ccc(Br)c2ccccc12] | 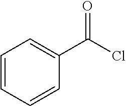 | 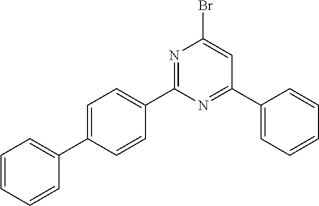 [SMILES: Brc1cc(-c2ccccc2)nc(-c2ccc(-c3ccccc3)cc2)n1] | 55% |
| 334 | 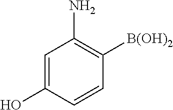 [SMILES: Nc1cc(O)ccc1B(O)O] | 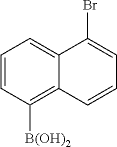 [SMILES: OB(O)c1cccc2c(Br)cccc12] | 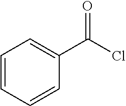 | 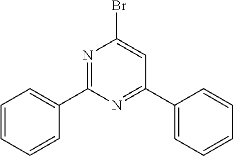 [SMILES: Brc1cc(-c2ccccc2)nc(-c2ccccc2)n1] | 47% |
| 348 | 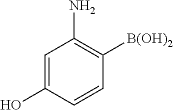 [SMILES: Nc1cc(O)ccc1B(O)O] | 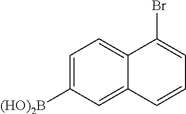 [SMILES: OB(O)c1ccc2c(Br)cccc2c1] | 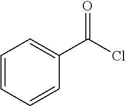 | 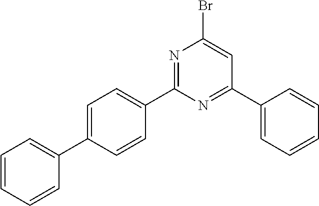 [SMILES: Brc1cc(-c2ccccc2)nc(-c2ccc(-c3ccccc3)cc2)n1] | 58% |
| 358 | 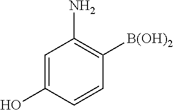 [SMILES: Nc1cc(O)ccc1B(O)O] | 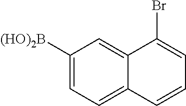 [SMILES: OB(O)c1ccc2cccc(Br)c2c1] | 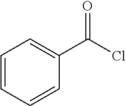 | 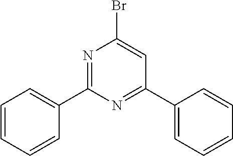 [SMILES: Brc1cc(-c2ccccc2)nc(-c2ccccc2)n1] | 58% |
| 376 | 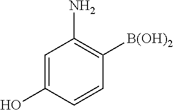 [SMILES: Nc1cc(O)ccc1B(O)O] | 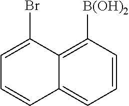 [SMILES: OB(O)c1cccc2cccc(Br)c12] | 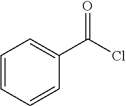 | 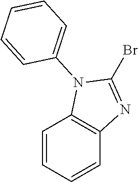 [SMILES: Brc1nc2ccccc2n1-c1ccccc1] | 52% |
| 381 | 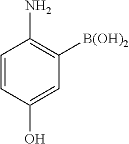 [SMILES: Nc1ccc(O)cc1B(O)O] | 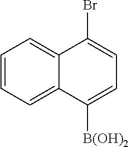 [SMILES: OB(O)c1ccc(Br)c2ccccc12] | 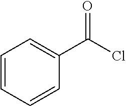 | 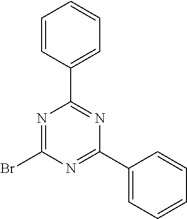 [SMILES: Brc1nc(-c2ccccc2)nc(-c2ccccc2)n1] | 63% |
| 396 | 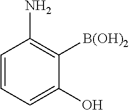 [SMILES: Nc1cccc(O)c1B(O)O] | 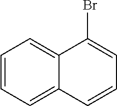 [SMILES: Brc1cccc2ccccc12] | 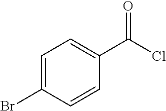 | 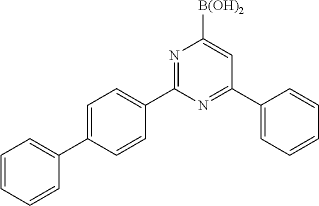 [SMILES: OB(O)c1cc(-c2ccccc2)nc(-c2ccc(-c3ccccc3)cc2)n1] | 55% |
| 455 | 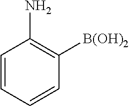 | 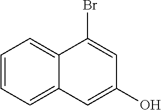 [SMILES: Oc1cc(Br)c2ccccc2c1] | 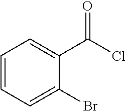 | 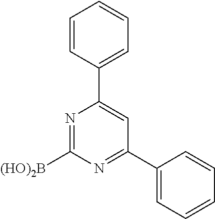 [SMILES: OB(O)c1nc(-c2ccccc2)cc(-c2ccccc2)n1] | 52% |
| 473 | 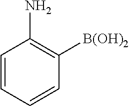 | 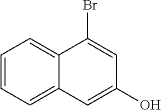 [SMILES: Oc1cc(Br)c2ccccc2c1] | 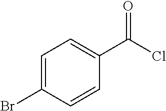 | 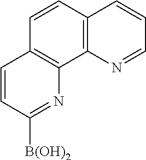 [SMILES: OB(O)c1ccc2ccc3cccnc3c2n1] | 58% |
| 501 | 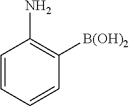 | 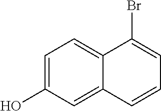 [SMILES: Oc1ccc2c(Br)cccc2c1] | 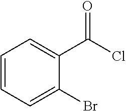 | 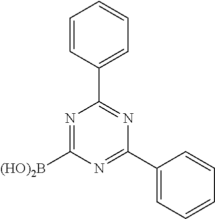 [SMILES: OB(O)c1nc(-c2ccccc2)nc(-c2ccccc2)n1] | 56% |

### [Preparation Example 4] Preparation of Compound 175

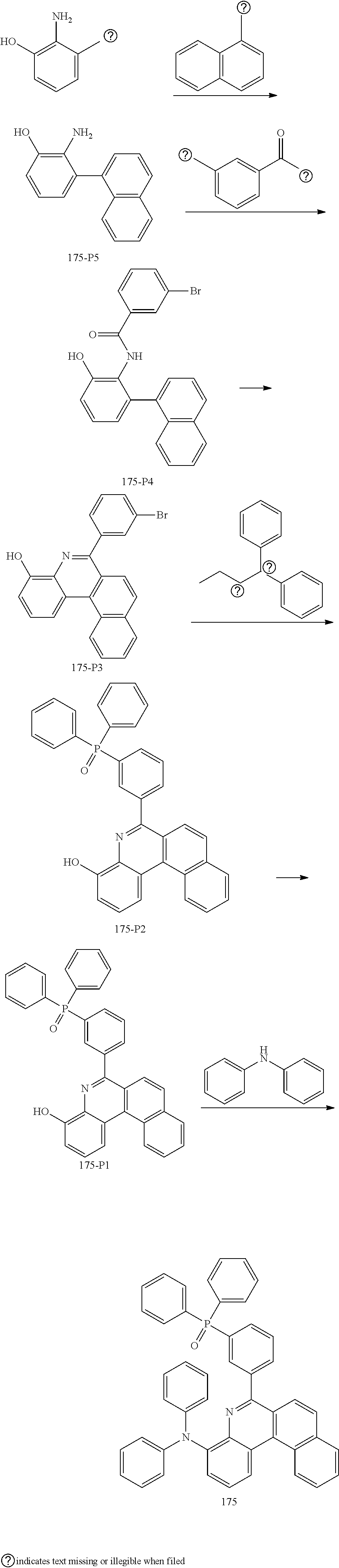

All compounds in Preparation Example 4 were synthesized in the same manner as described in Preparation Examples 1, 2 and 3.

A compound was synthesized in the same manner as in Preparation Example 4 using Intermediate A in the following Table 4 instead of (2-amino-3-hydroxyphenyl)boronic acid (A), Intermediate B instead of 1-bromonaphthalene (B), and Intermediate C instead of 3-bromobenzoyl chloride (C).

TABLE 4

| COM- | | | | |
|---|---|---|---|---|
| POUND | | | | |
| NO, | INTERMEDIATE A | INTERMEDIATE B | INTERMEDIATE C | YIELD |
| 319 | 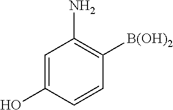 [SMILES: Nc1cc(O)ccc1B(O)O] | 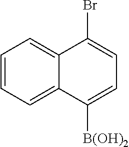 [SMILES: OB(O)c1ccc(Br)c2ccccc12] | 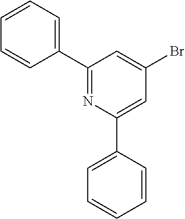 [SMILES: Brc1cc(-c2ccccc2)nc(-c2ccccc2)c1] | 54% |

### [Preparation Example 5] Preparation of Compound 401

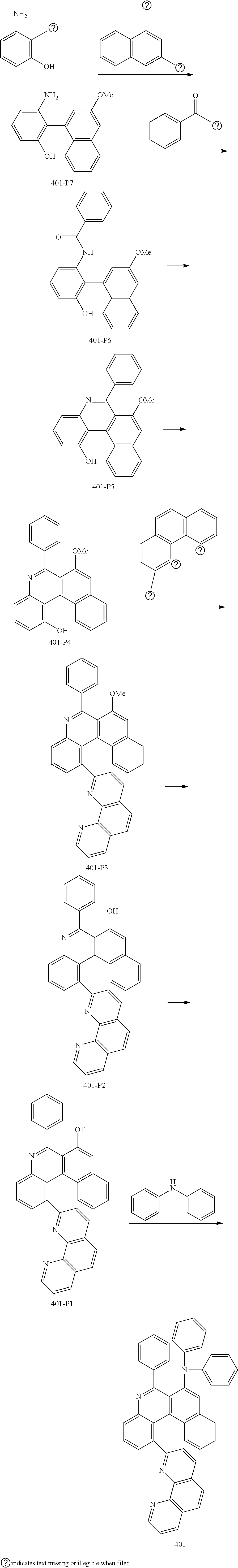

Synthesis was performed in the same manner as described in Preparation Examples 1, 2 and 3, except for the synthesis of Compound 401-P2 in Preparation Example 5.

1) Preparation of Compound 401-P2

After Compound 401-P3 (12.9 g, 25.12 mmol) was dissolved in dichloromethane, tribromoborane (5.96 mL, 62.79 mmol) was added at 0° C. at once, and then the resulting mixture was stirred at room temperature for 18 hours. After the reaction was completed, an aqueous Na₂CO₃ solution was added thereto at 0° C. to neutralize the resulting product, and then the resulting product was extracted with distilled water and EA. After the organic layer was dried over anhydrous MgSO₄, the solvent was removed by a rotary evaporator, and then the residue was purified by column chromatography using dichloromethane and hexane as an eluting solvent, thereby obtaining 10.5 g (84%) of Compound 401-P2.

A compound was synthesized in the same manner as in Preparation Example 5 using Intermediate A in the following Table 5 instead of (2-amino-6-hydroxyphenyl)boronic acid (A), Intermediate B instead of 1-bromo-3-methoxynaphthalene (B), Intermediate C instead of benzoyl chloride (C), and Intermediate D instead of (1,10-phenanthrolin-2-yl)boronic acid (D).

TABLE 5

| COM- | | | |
|---|---|---|---|
| POUND | | | |
| NO. | INTERMEDIATE A | INTERMEDIATE B | INTERMEDIATE C. |
| 421 | 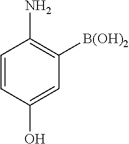 [SMILES: Nc1ccc(O)cc1B(O)O] | 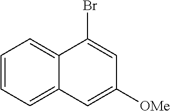 [SMILES: COc1cc(Br)c2ccccc2c1] | 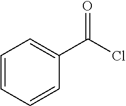 |
| 433 | 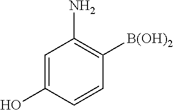 [SMILES: Nc1cc(O)ccc1B(O)O] | 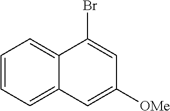 [SMILES: COc1cc(Br)c2ccccc2c1] | 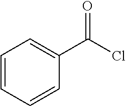 |
| 444 | 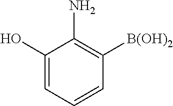 [SMILES: Nc1c(O)cccc1B(O)O] | 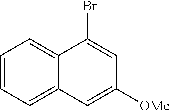 [SMILES: COc1cc(Br)c2ccccc2c1] | 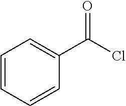 |
| 492 | 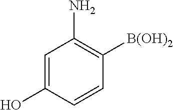 [SMILES: Nc1cc(O)ccc1B(O)O] | 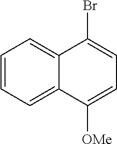 [SMILES: COc1ccc(Br)c2ccccc12] | 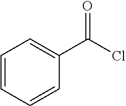 |
| 513 | 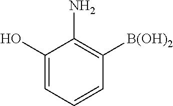 [SMILES: Nc1c(O)cccc1B(O)O] | 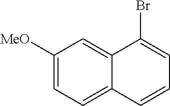 [SMILES: COc1ccc2cccc(Br)c2c1] | 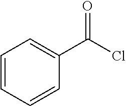 |
| 519 | 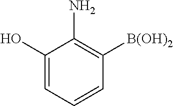 [SMILES: Nc1c(O)cccc1B(O)O] | 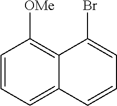 [SMILES: COc1cccc2cccc(Br)c12] | 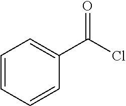 |

| COM- | | |
|---|---|---|
| POUND | | |
| NO. | INTERMEDIATE D | YIELD |
| 421 | 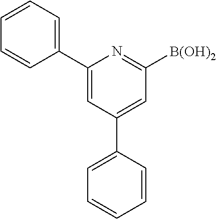 [SMILES: OB(O)c1cc(-c2ccccc2)cc(-c2ccccc2)n1] | 53% |
| 433 | 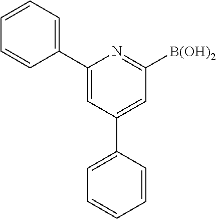 [SMILES: OB(O)c1cc(-c2ccccc2)cc(-c2ccccc2)n1] | 55% |
| 444 | 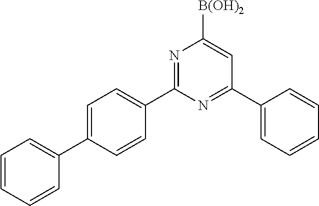 [SMILES: OB(O)c1cc(-c2ccccc2)nc(-c2ccc(-c3ccccc3)cc2)n1] | 55% |
| 492 | 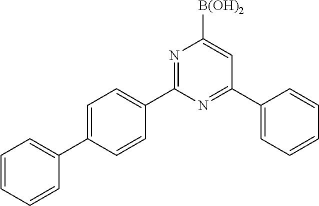 [SMILES: OB(O)c1cc(-c2ccccc2)nc(-c2ccc(-c3ccccc3)cc2)n1] | 52% |
| 513 | 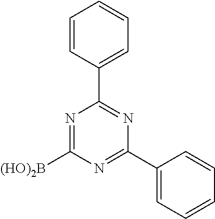 [SMILES: OB(O)c1nc(-c2ccccc2)nc(-c2ccccc2)n1] | 55% |
| 519 | 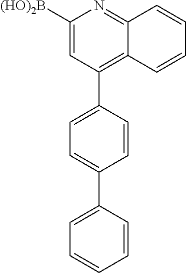 [SMILES: OB(O)c1cc(-c2ccc(-c3ccccc3)cc2)c2ccccc2n1] | 54% |

### [Preparation Example 6] Preparation of Compound 521

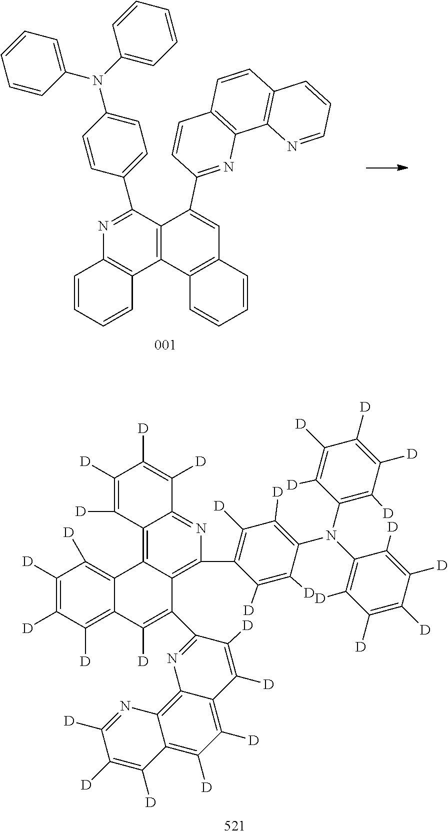 [SMILES: c1ccc(N(c2ccccc2)c2ccc(-c3nc4ccccc4c4c3c(-c3ccc5ccc6cccnc6c5n3)cc3ccccc34)cc2)cc1 ; [2H]c1nc2c(c([2H])c1[2H])c([2H])c([2H])c1c([2H])c([2H])c(-c3c([2H])c4c([2H])c([2H])c([2H])c([2H])c4c4c3c(-c3c([2H])c([2H])c(N(c5c([2H])c([2H])c([2H])c([2H])c5[2H])c5c([2H])c([2H])c([2H])c([2H])c5[2H])c([2H])c3[2H])nc3c([2H])c([2H])c([2H])c([2H])c34)nc12]

1) Preparation of Compound 521

After Compound 001 (10 g, 15.37 mmol), trifluoromethanesulfonic acid (3.46 g, 23.05 mmol) and 100 ml D6-benzene were put into a reaction flask, the resulting mixture was stirred under reflux for 5 hours. After the reaction was completed, the reaction was terminated by adding water thereto, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 521 (9 g, 84%).

### [Preparation Example 7] Preparation of Compound 602 and Compound 603

A compound was synthesized by the same method as in Production Example 6, except that the reaction temperature and time shown in Table 6 below were used instead of the reaction temperature and time shown in Production Example 6 above.

TABLE 6

| COMPOUND | REACTION | REACTION | |
|---|---|---|---|
| NO. | TEMPERATURE | TIME | YIELD |
| 602 | Reflux | 1 hour | 81% |
| 603 | 30° C. | 1 hour | 77% |

Compounds were prepared in the same manner as in the Preparation Examples, and the synthesis confirmation results thereof are shown in Tables 7 and 8. Table 7 shows the measured values of ¹H NMR (DMSO, 300 Mz), and Table 8 shows the measured values of field desorption mass spectrometry (FD-MS).

TABLE 7

| COMPOUND | ¹H NMR(DMSO, 300 MHz) |
|---|---|
| 001 | δ = 8.80(1H, d), 8.71(1H, d), 8.45(1H, d), 8.23-8.12(5H, m), 8.02- |
| | 7.90(5H, m), 7.70(1H, t), 7.59-7.56(3H, m), 7.37-7.24(7H, m), 7.08- |
| | 7.00(6H, m) |
| 003 | δ = 8.20(1H, d), 8.12(2H, d), 8.03-7.94(4H, m), 7.85-7.70(6H, m), 7.59- |
| | 7.51(8H, m), 7.37(2H, d), 7.24(4H, t), 7.08-7.00(6H, m) |
| 017 | δ = 8.97(2H, d), 8.36(4H, d), 8.21-8.20(2H, s), 8.12(2H, d), 7.94(1H, |
| | d), 7.85(1H, t), 7.70(1H, t), 7.59-7.50(8H, m), 7.37(2H, d), 7.24(4H, |
| | t), 7.08-7.00(6H, m) |
| 025 | δ = 9.07(1H, d), 8.80(1H, d), 8.71(1H, d), 8.49-8.45(2H, m), 8.20- |
| | 8.14(4H, d), 8.00-7.85(5H, m), 7.70-7.56(3H, m), 7.37-7.24(7H, m), |
| | 7.08-7.00(6H, m) |
| 027 | δ = 9.22(1H, d), 8.20(1H, d), 8.12(2H, d), 8.01-7.94(3H, m), 7.85- |
| | 7.62(8H, m), 7.51(6H, s), 7.37(2H, d), 7.24(4H, t), 7.08-7.00(6H, m) |
| 041 | δ = 9.09(1H, s), 8.49(1H, d), 8.36(4H, d), 8.20-8.17(4H, m), 7.99- |
| | 7.94(2H, m), 7.85(1H, t), 7.70-7.62(2H, m), 7.50(6H, t), 7.37(2H, d), |
| | 7.24(4H, t), 7.08-7.00(6H, m) |
| 047 | δ = 8.85(1H, s), 8.37(1H, d), 8.20-8.12(5H, m), 7.99-7.94(3H, m), |
| | 7.85(1H, t), 7.75-7.65(6H, m), 7.49-7.37(5H, m), 7.25-7.24(8H, m), |
| | 7.15(1H, s), 7.08-7.00(6H, m) |
| 054 | δ = 8.46(1H, s), 8.35(2H, d), 8.23-8.12(4H, m), 7.94-7.85(7H, m), 7.70- |
| | 7.49(8H, m), 7.37(2H, d), 7.24(4H, t), 7.08(6H, m) |
| 072 | δ = 8.56(1H, d), 8.25-8.10(5H, m), 8.00-7.94(3H, m), 7.85-2H, m), |
| | 7.70-7.24(15H, m), 7.08-7.00(6H, m) |
| 079 | δ = 8.54(1H, d), 8.24-8.23(2H, s), 8.12-7.94(10H, m), 7.62-7.49(9H, m), |
| | 7.37(2H, d), 7.24(4H, t), 7.08-7.00(6H, m) |
| 092 | δ = 8.54(1H, d), 8.43(1H, d), 8.25-8.23(2H, d), 8.12-8.08(3H, m), 7.99- |
| | 7.94(6H, m), 7.75(2H, d), 7.62-7.37(11H, m), 7.25-7.24(6H, m), 7.08- |
| | 7.00(6H, m) |
| 102 | δ = 8.54(1H, d), 8.39-8.35(4H, m), 8.24(1H, d), 8.23(1H, s), 8.12(2H, |
| | d), 7.99-7.94(4H, m), 7.62-7.49(9H, m), 7.37(2H, d), 7.24(4H, t), 7.08- |
| | 7.00(6H, m) |
| 115 | δ = 8.54(1H, d), 8.43(1H, d), 8.30(1H, d), 8.23(1H, s), 8.12(2H, d), |
| | 7.99-7.88(7H, m), 7.62-7.49(9H, m), 7.37(2H, d), 7.24(4H, t), 7.08- |
| | 7.00(6H, m) |
| 123 | δ = 9.22(1H, d), 8.20(1H, d), 7.99-7.51(20H, m), 7.24-7.18(4H, m), |
| | 7.08-7.00(6H, m) |
| 137 | δ = 9.37(1H, d), 8.49-8.46(2H, m), 8.36(4H, d), 8.20(1H, d), 7.99- |
| | 7.85(4H, m), 7.70-7.62(2H, m), 7.50(6H, t), 7.39-7.37(2H, m), 7.24 (4H, |
| | t), 7.09-7.00(7H, m) |
| 146 | δ = 8.97(1H, d), 8.69(2H, d), 8.40(1H, s), 8.20(1H, d), 8.12(1H, d), |
| | 7.94(1H, d), 7.85-7.70(8H, m), 7.59-7.51(10H, m), 7.37(2H, d), |
| | 7.24(4H, t), 7.08-7.00(6H, m) |
| 154 | δ = 9.04(1H, d), 8.80(1H, d), 8.71(1H, d), 8.54(1H, d), 8.45(1H, d), |
| | 8.33-8.30(3H, m), 8.20(1H, d), 7.99(2H, d), 7.90-7.88(2H, m), 7.73(1H, |
| | t), 7.62-7.53(7H, m), 7.37-7.24(7H, m), 7.08-7.00(6H, m) |
| 161 | δ = 8.80(1H, d), 8.71(1H, d), 8.54(1H, d), 8.45(1H, d), 8.35-8.33(3H, |
| | m), 8.20(1H, d), 7.99(2H, d), 7.90-7.86(2H, m), 7.65-7.53(7H, m), 7.29- |
| | 7.24(5H, m), 7.08-7.00(6H, m) |
| 175 | δ = 8.98(1H, d), 8.64(1H, d), 8.16-8.13(2H, s), 7.92-7.87(3H, m), 7.77- |
| | 7.51(17H, m), 7.24(4H, t), 7.08-7.00(6H, m) |
| 192 | δ = 8.69(2H, d), 8.54(1H, d), 8.33-8.30(3H, m), 8.23(1H, s), 7.99- |
| | 7.86(7H, m), 7.75(2H, d), 7.65-7.41(10H, m), 7.25-7.24(6H, m), 7.08- |
| | 7.00(6H, m) |
| 194 | δ = 8.69(2H, d), 8.54(1H, d), 8.33-8.29(5H, m), 8.20(2H, s), 7.99(2H, |
| | d), 7.86-7.85(3H, m), 7.65-7.49(10H, m), 7.24(4H, t), 7.08-7.00(6H, m) |
| 207 | δ = 8.33(1H, d), 8.24-8.18(4H, m), 8.02-7.98(3H, m), 7.86(1H, d), 7.75- |
| | 7.59(9H, m), 7.49-7.41(4H, m), 7.25-7.24(8H, m), 7.15(1H, s), 7.08- |
| | 7.00(6H, m) |
| 218 | δ = 8.97(2H, d), 8.33-8.19(10H, m), 7.86(1H, d), 7.65-7.49(12H, m), |
| | 7.24(4H, t), 7.08-7.00(6H, m) |
| 226 | δ = 9.07(1H, d), 8.35-8.33(3H, d), 8.24-8.19(3H, m), 8.10(1H, d), 8.00- |
| | 7.86(5H, m), 7.65-7.49(11H, m), 7.24(4H, t), 7.08-7.00(6H, m) |
| 239 | δ = 9.09(1H, s), 8.49(1H, d), 8.33(1H, d), 8.23-8.17(4H, m), 7.99- |
| | 7.86(6H, m), 7.65-7.49(11H, m), 7.24(4H, t), 7.08-7.00(6H, m) |
| 245 | δ = 8.85-8.80(2H, m), 8.71(1H, d), 8.45(1H, d), 8.37-8.33(2H, d), 8.24- |
| | 8.19(3H, m), 7.99-7.86(4H, m), 7.65-7.49(6H, m), 7.29(1H, d), 7.24(4H, |
| | t), 7.08-7.00(6H, m) |
| 253 | δ = 8.85(1H, s), 8.33-8.19(8H, m), 7.99-7.86(3H, m), 7.75-7.41(13H, m), |
| | 7.24(4H, t), 7.08-7.00(6H, m) |
| 268 | δ = 8.56(1H, d), 8.33(1H, d), 8.25-8.19(3H, m), 8.18(1H, d), 8.00- |
| | 7.99(2H, m), 7.86-7.81(2H, m), 7.65-7.48(9H, m), 7.38(2H, d), 7.28(1H, |
| | t), 7.24(4H, t), 7.08-7.00(6H, m) |
| 274 | δ = 8.54(1H, d), 8.36(1H, d), 8.35(3H, d), 8.23(1H, s), 7.99-7.94(4H, |
| | m), 7.62-7.50(12H, m), 7.24(4H, t), 7.13-7.00(7H, m) |
| 278 | δ = 8.54(1H, d), 8.36-8.29(6H, m), 8.20(2H, s), 7.99-7.96(3H, m), 7.61- |
| | 7.53(12H, m), 7.24(4H, t), 7.13-7.00(7H, m) |
| 281 | δ = 8.80(1H, d), 8.72(1H, 8.71(1H, d), 8.54(1H, d), 8.45(1H, d), |
| | 8.36-8.33(3H, m), 8.20(1H, m), 7.99(2H, d), 7.90(1H, d), 7.73(1H, t), |
| | 7.62-7.53(5H, m), 7.29-7.24(5H, m), 7.13-7.00(7H, m) |
| 300 | δ = 8.69(2H, d), 8.54(1H, d), 8.36(1H, d), 8.30(2H, d), 8.23(1H, s), |
| | 7.99-7.94(6H, m), 7.75(2H, d), 7.62-7.41(10H, m), 7.25-7.24(6H, m), |
| | 7.13-7.00(7H, m) |
| 302 | δ = 8.69(2H, d), 8.54(1H, d), 8.36(1H, d), 8.29(4H, d), 8.20(2H, s), |
| | 7.99(2H, d), 7.85(2H, d), 7.62-7.49(10H, m), 7.24(4H, t), 7.13-7.00(7H, |
| | m) |
| 314 | δ = 8.36-8.19(9H, m), 8.02(1H, d), 7.98(1H, d), 7.84(1H, s), 7.65- |
| | 7.49(12H, m), 7.24(4H, t), 7.13-7.00(7H, m) |
| 319 | δ = 8.97(1H, d), 8.40-8.36(2H, m), 8.19-8.12(3H, d), 7.77(4H, d), 7.65- |
| | 7.49(12H, m), 7.24(4H, t), 7.13-7.00(7H, m) |
| 324 | δ = 8.97(2H, d), 8.36(1H, d), 8.23-8.19(4H, m), 7.96-7.94(4H, m), |
| | 7.75(2H, d), 7.65-7.41(12H, m), 7.25(2H, d), 7.24(4H, t), 7.13-7.00(7H, m) |
| 334 | δ = 9.07(1H, d), 8.36-8.35(3H, d), 8.23-8.19(3H, d), 8.10(1H, d), 8.00- |
| | 7.94(4H, m), 7.65(11H, m), 7.24(4H, t), 7.13-7.00(7H, m) |
| 348 | δ = 8.46(1H, s), 8.36(1H, d), 8.23-8.17(4H, m), 7.99-7.94(6H, m), |
| | 7.75(2H, d), 7.65-7.41(11H, m), 7.25(2H, d), 7.24(4H, t), 7.13-7.00(7H, |
| | m) |
| 358 | δ = 8.46(1H, s), 8.36-8.35(3H, d), 8.23(1H, s), 8.19(2H, d), 7.99- |
| | 7.93(5H, m), 7.65-7.49(11H, m), 7.24(4H, t), 7.13-7.00(7H, m) |
| 376 | δ = 8.56(1H, d), 8.36(1H, d), 8.25-8.19(3H, d), 8.10(1H, d), 8.00- |
| | 7.99(2H, d), 7.81(1H, d), 7.65-7.48(9H, m), 7.38(2H, d), 7.28-7.24(5H, |
| | m), 7.13-7.00(7H, m) |
| 381 | δ = 8.97(2H, d), 8.55(1H, d), 8.36(4H, d), 8.21(1H, s), 8.19(2H, d), |
| | 7.65-7.44(12H, m), 7.24(4H, t), 7.08-7.00(6H, m), 6.81(1H, s) |
| 396 | δ = 8.69(2H, d), 8.54(1H, d), 8.30(2H, d), 8.23(1H, s), 8.14(1H, d), |
| | 7.99-7.96(6H, m), 7.84(1H, d), 7.75(2H, d), 7.62-7.41(10H, m), 7.25(2H, |
| | d), 7.24(4H, t), 7.08-7.00(6H, m) |
| 401 | δ = 8.80(1H, d), 8.71(1H, d), 8.63(1H, d), 8.54(1H, d), 8.45(1H, d), |
| | 8.20-8.04(6H, m), 7.9(1H, d), 7.69-7.49(6H, m), 7.29-7.24(6H, m), 7.08- |
| | 7.00(6H, m) |
| 421 | δ = 8.82(1H, d), 8.54(1H, d), 8.47(1H, s), 8.29-8.11(8H, m), 7.75- |
| | 7.41(13H, m), 7.28-7.24(5H, m), 7.08-7.00(6H, m) |
| 433 | δ = 8.76(1H, s), 8.63(1H, d), 8.54(1H, d), 8.39(1H, d), 8.29(2H, d), |
| | 8.20-8.11(5H, m), 7.75-7.41(13H, m), 7.28-7.24(5H, m), 7.08-7.00(6H, m) |
| 444 | δ = 8.65(1H, d), 8.54(1H, d), 8.30(1H, d), 8.23-8.11(4H, m), 7.96- |
| | 7.88(5H, m), 7.75-7.41(13H, m), 7.28-7.24(7H, m), 7.08-7.00(6H, m) |
| 455 | δ = 8.54(1H, d), 8.35(1H, d), 8.23(1H, s), 8.20(1H, d), 8.11(1H, d), |
| | 7.96-7.85(7H, m), 7.70-7.49(11H, m), 7.28-7.24(5H, m), 7.08-7.00(6H, m) |
| 473 | δ = 8.80(1H, d), 8.71-8.69(5H, m), 8.54(1H, d), 8.45(1H, d), 8.20(2H, |
| | d), 8.11(1H, d), 7.94-7.85(3H, m), 7.70-7.56(4H, m), 7.29-7.24(6H, m), |
| | 7.08-7.00(6H, m) |
| 492 | δ = 8.54(1H, d), 8.39-8.37(2H, m), 8.24-8.16(5H, m), 7.96-7.94(4H, d), |
| | 7.75-7.41(13H, m), 7.25(2H, d), 7.24(4H, t), 7.08-6.94(7H, m) |
| 501 | δ = 8.36-8.35(5H, d), 8.20(1H, d), 7.99-7.94(3H, m), 7.85(1H, t), 7.70- |
| | 7.50(11H, m), 7.36(1H, d), 7.24(4H, t), 7.11-7.00(7H, m) |
| 513 | δ = 8.46-8.30(6H, m), 8.19(2H, d), 7.99(1H, d), 7.88(1H, t), 7.65- |
| | 7.62(3H, m), 7.54-7.42(9H, m), 7.24(4H, t), 7.11-7.00(7H, m) |
| 519 | δ = 9.04(1H, d), 8.30(1H, d), 8.19-8.18(3H, m), 7.99(2H, d), 7.88- |
| | 7.41(15H, m), 7.25(4H, d), 7.24(4H, t), 7.08-7.00(6H, m) |
| 521 | [symbol] |
| 569 | δ = 8.95(1H, d), 8.33-8.20(10H, m), 8.00-7.94(3H, m), 7.85(1H, t), |
| | 7.73-7.49(12H, m) 7.37(2H, d), 7.24(4H, t), 7.08-7.00(6H, m) |
| 571 | δ = 8.80(1H, d), 8.71(1H, t), 8.45(1H, d), 8.23-8.12(5H, m), 8.02- |
| | 7.85(5H, m), 7.75-7.70(3H, m), 7.59-7.24(15H, m), 7.08(3H, t) |
| 576 | δ = 8.46(1H, s), 8.35(1H, d), 8.23(1H, s), 8.20(1H, d), 8.12(2H, d), |
| | 7.99-7.85(7H, m), 7.75-7.87(19H, m), 7.24(2H, t), 7.08-7.00(3H, m) |
| 583 | δ = 9.22(1H, d), 8.20(1H, d), 8.12(2H, d), 8.01-7.94(3H, m), 7.85- |
| | 7.62(10H, m), 7.54-7.37(12H, m), 7.24(2H, t), 7.11-7.00(3H, m) |
| 586 | δ = 8.35(2H, d), 8.23(1H, s), 8.25-8.10(5H, m), 8.00(1H, t), 7.99(1H, |
| | d), 7.94-7.37(26H, m), 7.11(2H, s) |
| 591 | δ = 8.85(1H, s), 8.80(1H, d), 8.71 (1H, d), 8.45-8.37(3H, d), 8.20- |
| | 8.12(4H, d), 7.99-7.85(9H, m), 7.70-7.24(11H, m), 7.08-7.00(3H, m) |
| 596 | δ = 8.56(1H, s), 8.54(1H, d), 8.40(1H, d), 8.22(1H, s), 8.12(2H, d), |
| | 7.99-7.97(4H, m), 7.77(4H, d), 7.62-7.51(11H, m), 7.39-7.24(6H, m), |
| | 7.08-6.97(4H, m) |
| 598 | δ = 8.49(1H, d), 8.46(11H, s), 8.36-8.33(6H, d), 8.22-8.20(2H, m), 7.99- |
| | 7.85(5H, m), 7.73-7.50(14H, m), 7.39-7.24(6H, m), 7.08-6.97(4H, m) |
| 599 | δ = 8.76(1H, s), 8.63(1H, d), 8.54(1H, d), 8.45-8.29(6H, m), 8.20(2H, |
| | s), 7.99-7.93(4H, m), 7.85(1H, d), 7.75-7.73(3H, m), 7.61-7.37(17H, m), |
| | 7.24(2H, t), 7.08-7.00(3H, m) |
| 602 | δ = 8.97(1H, d), 8.40(1H, s), 8.20-8.12(4H, d), 7.94(1H, d), 7.85(1H, |
| | t), 7.70(1H, t), 7.59(2H, t), 7.37(2H, d) |
| 603 | δ = 8.80(1H, d), 8.71(1H, d), 8.45(1H, d), 8.20(1H, d), 7.90(1H, d), |
| | 7.75(2H, d), 7.56-7.24(11H, m), 7.08-7.00(3H, m)1 |

TABLE 8

| COMPOUND | FD-MS | COMPOUND | FD-MS |
|---|---|---|---|
| 001 | m/z = 650.25 | 003 | m/z = 672.23 |
| | (C47H30N4 = 650.78) | | (C47H33N2OP = 672.77) |
| 017 | m/z = 703.27 | 025 | m/z = 650.25 |
| | (C50H33N5 = 703.85) | | (C47H30N4 = 650.78) |
| 027 | m/z = 672.23 | 041 | m/z = 703.27 |
| | (C47H33N2OP = 672.77) | | (C50H33N5 = 703.85) |
| 047 | m/z = 751.30 | 054 | m/z = 702.28 |
| | (C56H37N3 = 751.93) | | (C51H34N4 = 702.86) |
| 072 | m/z = 664.26 | 079 | m/z = 702.28 |
| | (C48H32N4 = 664.81) | | (C51H34N4 = 702.86) |
| 092 | m/z = 778.31 | 102 | m/z = 702.28 |
| | (C57H38N4 = 778.96) | | (C51H34N4 = 702.86) |
| 115 | m/z = 702.28 | 123 | m/z = 672.23 |
| | (C51H34N4 = 702.88) | | (C47H33N2OP = 672.77) |
| 137 | m/z = 703.27 | 146 | m/z = 748.26 |
| | (C50H33N5 = 703.85) | | (C53H37N2OP = 748.87) |
| 154 | m/z = 726.28 | 161 | m/z = 650.25 |
| | (C53H34N4 = 726.88) | | (C47H30N4 = 650.78) |
| 175 | m/z = 671.24 | 192 | m/z = 778.31 |
| | (C48H34NOP = 671.78) | | (C57H38N4 = 778.96) |
| 194 | m/z = 701.28 | 207 | m/z = 751.30 |
| | (C52H35N3 = 701.87) | | (C56H37N3 = 751.93) |
| 218 | m/z = 701.28 | 226 | m/z = 702.28 |
| | (C52H35N3 = 701.87) | | (C51H34N4 = 702.86) |
| 239 | m/z = 702.28 | 245 | m/z = 650.25 |
| | (C51H34N4 = 702.86) | | (C47H30N4 = 650.78) |
| 253 | m/z = 701.28 | 268 | m/z = 664.26 |
| | (C52H35N3 = 701.87) | | (C48H32N4 = 664.81) |
| 274 | m/z = 702.28 | 278 | m/z = 701.28 |
| | (C51H34N4 = 702.86) | | (C52H35N3 = 701.87) |
| 281 | m/z = 650.25 | 300 | m/z = 778.31 |
| | (C47H30N4 = 650.78) | | (C57H38N4= 778.96) |
| 302 | m/z = 701.28 | 314 | m/z = 701.28 |
| | (C52H35N3 = 701.87) | | (C52H35N3 = 701.87) |
| 319 | m/z = 672.23 | 324 | m/z = 778.31 |
| | (C47H33N2OP = 672.77) | | (C57H38N4 = 778.96) |
| 334 | m/z = 702.28 | 348 | m/z = 778.31 |
| | (C51H34N4 = 702.86) | | (C57H38N4 = 778.96) |
| 358 | m/z = 702.28 | 376 | m/z = 664.26 |
| | (C51H34N4 = 702.86) | | (C48H32N4 = 664.81) |
| 381 | m/z = 703.27 | 396 | m/z = 778.31 |
| | (C50H33N5 = 703.85) | | (C57H38N4 = 778.96) |
| 401 | m/z = 850.25 | 421 | m/z = 701.28 |
| | (C47H30N4 = 650.78) | | (C52H35N3 = 701.87) |
| 433 | m/z = 701.28 | 444 | m/z = 778.31 |
| | (C52H35N3 = 701.87) | | (C57H38N4 = 778.96) |
| 455 | m/z = 702.28 | 473 | m/z = 650.25 |
| | (C51H34N4 = 702.86) | | (C47H30N4 = 650.78) |
| 492 | m/z = 778.31 | 501 | m/z = 703.27 |
| | (C57H38N4 = 778.96) | | (C50H33N5 = 703.85) |
| 513 | m/z = 709.27 | 519 | m/z = 751.30 |
| | (C50H33N5 = 703.85) | | (C56H37N3 = 751.93) |
| 521 | m/z = 680.44 | 569 | m/z = 777.31 |
| | (C47D30N4 = 680.97) | | (C58H39N3 = 777.97) |
| 571 | m/z = 726.88 | 576 | m/z = 778.31 |
| | (C53H34N4 = 726.28) | | (C57H3\|8N4 = 778.96) |
| 583 | m/z = 722.83 | 586 | m/z = 802.98 |
| | (C51H35N2OP = 722.25) | | (C59H38N4 = 802.31) |
| 591 | m/z = 756.93 | 596 | m/z = 762.85 |
| | (C53H32N4S = 756.23) | | (C53H35N2O2P = 762.24) |
| 598 | m/z = 870.03 | 599 | m/z = 884.11 |
| | (C62H39N5O = 869.32) | | (C64H41N3S = 883.30) |
| 602 | m/z = 773.01 | 603 | m/z = 739.96 |
| | (C53H13D24N2OP = 772.41) | | (C53H21D13N4 = 739.36) |

### Experimental Example 1

### Comparative Example 1

1) Manufacture of Organic Light Emitting Device

Trichloroethylene, acetone, ethanol, and distilled water were each sequentially used to ultrasonically wash a transparent electrode indium tin oxide (ITO) thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

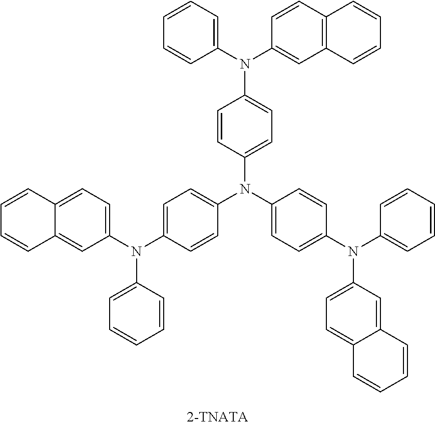 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

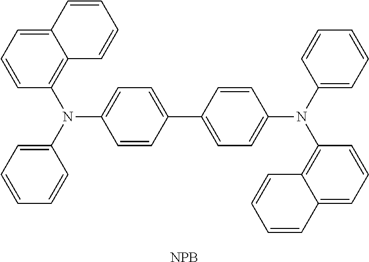 [SMILES: c1ccc(N(c2ccc(-c3ccc(N(c4ccccc4)c4cccc5ccccc45)cc3)cc2)c2cccc3ccccc23)cc1 ; "NPB"]

The hole injection layer and the hole transport layer were formed as described above, and then a blue light emitting material having the following structure as a light emitting layer was deposited thereon. Specifically, a blue light emitting host material H1 was vacuum deposited to have a thickness of 200 Å on one cell in the vacuum deposition apparatus, and a blue light emitting dopant material D1 was vacuum deposited thereon in an amount of 5% based on the host material.

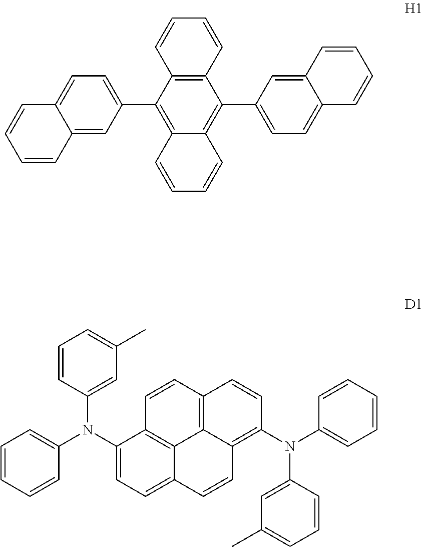

Subsequently, a compound having the following structural formula E1 as an electron transport layer was deposited to have a thickness of 300 Å.

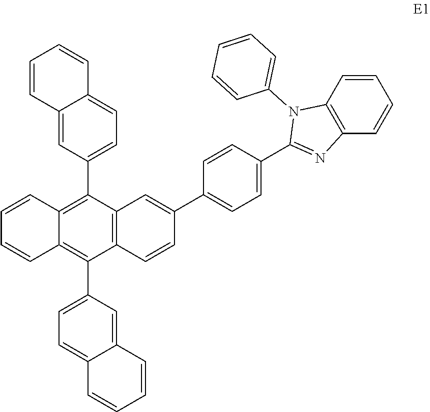 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10⁻⁸ to 10⁻⁶ torr for each material, and used for the manufacture of OLED.

2) Driving Voltage and Light Emitting Efficiency of Organic Electroluminescence Device

An organic electroluminescence device was manufactured in the same manner as in Comparative Example 1, except that the compound shown in the following Table 9 was used instead of E1 used when an electron transport layer was formed in Comparative Example 1.

For the organic electroluminescence device manufactured as described above, electroluminescence (EL) characteristics were measured by M7000 manufactured by McScience Inc., and based on the measurement result thereof, T₉₅ was measured by a service life measurement device (M6000) manufactured by McScience Inc., when the reference luminance was 3,500 cd/m². The results of measuring the driving voltage, light emitting efficiency, color coordinate (CIE), and service life (T₉₅) of the blue organic electroluminescence device manufactured according to the present invention are shown as in Table 9.

TABLE 9

| | | | LIGHT | | SER- |
|---|---|---|---|---|---|
| | | DRIVING | EMITTING | | VICE |
| | | VOLTAGE | EFFICIENCY | CIE | LIFE |
| | COMPOUND | (V) | (cd/A) | (x, y) | (T₉₅) |
| EXAMPLE 1 | 1 | 4.38 | 6.86 | (0.133, 0.100) | 75 |
| EXAMPLE 2 | 3 | 4.42 | 6.87 | (0.134, 0.100) | 77 |
| EXAMPLE 3 | 17 | 4.47 | 6.88 | (0.134, 0.100) | 76 |
| EXAMPLE 4 | 25 | 4.44 | 6.85 | (0.134, 0.101) | 71 |
| EXAMPLE 5 | 27 | 4.44 | 6.87 | (0.133, 0.101) | 72 |
| EXAMPLE 6 | 41 | 4.49 | 6.71 | (0.134, 0.100) | 68 |
| EXAMPLE 7 | 47 | 4.41 | 6.90 | (0.133, 0.101) | 69 |
| EXAMPLE 8 | 54 | 4.42 | 6.88 | (0.134, 0.102) | 75 |
| EXAMPLE 9 | 72 | 4.44 | 6.88 | (0.134, 0.101) | 77 |
| EXAMPLE 10 | 79 | 4.41 | 6.80 | (0.134, 0.101) | 71 |
| EXAMPLE 11 | 92 | 4.43 | 6.70 | (0.133, 0.100) | 79 |
| EXAMPLE 12 | 102 | 4.48 | 6.85 | (0.134, 0.100) | 80 |
| EXAMPLE 13 | 123 | 4.47 | 6.85 | (0.134, 0.100) | 72 |
| EXAMPLE 14 | 146 | 4.48 | 6.84 | (0.133, 0.100) | 69 |
| EXAMPLE 15 | 154 | 4.45 | 6.81 | (0.134, 0.101) | 78 |
| EXAMPLE 16 | 161 | 4.45 | 6.78 | (0.134, 0.101) | 72 |
| EXAMPLE 17 | 192 | 4.47 | 6.80 | (0.133, 0.100) | 77 |
| EXAMPLE 18 | 194 | 4.41 | 6.83 | (0.133, 0.101) | 77 |
| EXAMPLE 19 | 207 | 4.42 | 6.73 | (0.134, 0.100) | 75 |
| EXAMPLE 20 | 239 | 4.43 | 6.85 | (0.134, 0.100) | 74 |
| EXAMPLE 21 | 253 | 4.39 | 6.90 | (0.133, 0.101) | 69 |
| EXAMPLE 22 | 268 | 4.47 | 6.74 | (0.134, 0.101) | 73 |
| EXAMPLE 23 | 274 | 4.49 | 6.93 | (0.133, 0.101) | 73 |
| EXAMPLE 24 | 278 | 4.48 | 6.95 | (0.134, 0.100) | 68 |
| EXAMPLE 25 | 281 | 4.44 | 6.88 | (0.133, 0.101) | 79 |
| EXAMPLE 26 | 300 | 4.46 | 6.90 | (0.133, 0.100) | 76 |
| EXAMPLE 27 | 302 | 4.41 | 6.85 | (0.134, 0.100) | 74 |
| EXAMPLE 28 | 334 | 4.47 | 6.83 | (0.134, 0.100) | 74 |
| EXAMPLE 29 | 348 | 4.48 | 6.87 | (0.134, 0.100) | 72 |
| EXAMPLE 30 | 358 | 4.44 | 6.88 | (0.134, 0.100) | 75 |
| EXAMPLE 31 | 376 | 4.42 | 6.85 | (0.134, 0.101) | 71 |
| EXAMPLE 32 | 381 | 4.43 | 6.87 | (0.133, 0.101) | 76 |
| EXAMPLE 33 | 421 | 4.43 | 6.71 | (0.134, 0.100) | 73 |
| EXAMPLE 34 | 433 | 4.48 | 6.90 | (0.133, 0.101) | 72 |
| EXAMPLE 35 | 444 | 4.49 | 6.85 | (0.134, 0.102) | 71 |
| EXAMPLE 36 | 455 | 4.51 | 6.88 | (0.134, 0.101) | 71 |
| EXAMPLE 37 | 473 | 4.52 | 6.71 | (0.134, 0.101) | 69 |
| EXAMPLE 38 | 492 | 4.37 | 6.82 | (0.133, 0.101) | 70 |
| EXAMPLE 39 | 501 | 4.41 | 6.75 | (0.134, 0.100) | 70 |
| EXAMPLE 40 | 513 | 4.45 | 6.77 | (0.133, 0.101) | 75 |
| EXAMPLE 41 | 569 | 4.42 | 6.81 | (0.134, 0.102) | 74 |
| EXAMPLE 42 | 583 | 4.38 | 6.85 | (0.133, 0.101) | 71 |
| EXAMPLE 43 | 596 | 4.43 | 6.82 | (0.134, 0.100) | 76 |
| EXAMPLE 44 | 598 | 4.41 | 6.79 | (0.133, 0.101) | 73 |
| COMPARATIVE | E1 | 5.51 | 6.17 | (0.134, 0.100) | 31 |
| EXAMPLE 1 | | | | | |
| COMPARATIVE | COMPARATIVE | 5.53 | 6.37 | (0.134, 0.100) | 60 |
| EXAMPLE 2 | COMPOUND A | | | | |
| COMPARATIVE | COMPARATIVE | 5.14 | 3.21 | (0.148, 0.177) | 62 |
| EXAMPLE 3 | COMPOUND B | | | | |
| COMPARATIVE | COMPARATIVE | 4.72 | 6.53 | (0.134, 0.102) | 66 |
| EXAMPLE 4 | COMPOUND C | | | | |

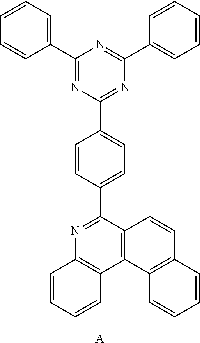

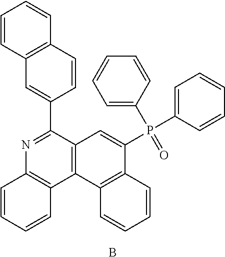

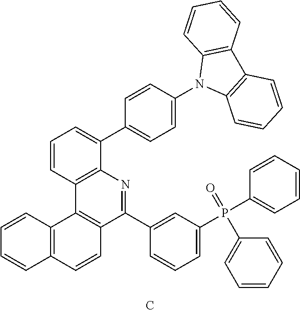

As can be seen from the results in Table 9, the organic light emitting device using an electron transport layer material of the blue organic light emitting device of the present invention has a low driving voltage and notably improved light emitting efficiency and service life compared to Comparative Examples 1 to 4. It is determined that these results are because due to the appropriate length and strength of a material, and the stable bond between the metals used for the negative electrode and the heterocyclic compound used in the present invention, the more stabilized compound can efficiently transfer electrons without the decomposition or destruction of the compound Therefore, it is determined that the compound of the present invention enhances electron-transporting characteristics or stability to bring excellence in terms of all the aspects in driving, efficiency, and service life.

### Experimental Example 2

1) Manufacture of Organic Light Emitting Device

A glass substrate, in which ITO was thinly coated to have a thickness of 1500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in order to implement an ITO work function and remove a residual film in a vacuum state, and thus, was transferred to a thermal deposition equipment for organic deposition.

An organic material having a 2-stack white organic light device (WOLED) structure was formed on the ITO transparent electrode (positive electrode). For a first stack, a hole transport layer was first formed by thermally vacuum depositing TAPC to have a thickness of 300 Å. The hole transport layer was formed, and then a light emitting layer was thermally vacuum deposited thereon as follows. The light emitting layer was deposited to have a thickness of 300 Å by doping a host TCz1 with a blue phosphorescent dopant FIrpic at a concentration of 8%. An electron transport layer was formed to have a thickness of 400 Å by using TmPyPB, and then a charge generation layer was formed to have a thickness of 100 Å by doping the compound described in the following Table 10 with Cs₂CO₃ at a concentration of 20%.

For a second stack, a hole injection layer was first formed by thermally vacuum depositing MoO₃ to have a thickness of 50 Å. A hole transport layer, which is a common layer, was formed to have a thickness of 100 Å by doping TAPC with MoO₃ at a concentration of 20%, and then formed by depositing TAPC to have a thickness of 300 Å. A light emitting layer was deposited thereon to have a thickness of 300 Å by doping a host TCz1 with a green phosphorescent dopant Ir(ppy)₃ at a concentration of 8%, and then formed to have a thickness of 600 Å using TmPyPB as an electron transport layer. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁸ to 10⁻⁶ torr for each material, and used for the manufacture of OLED.

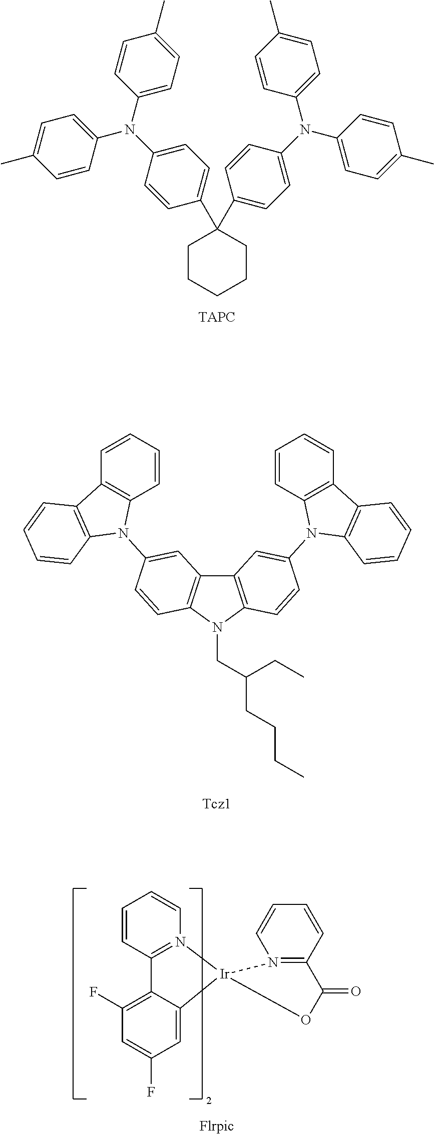

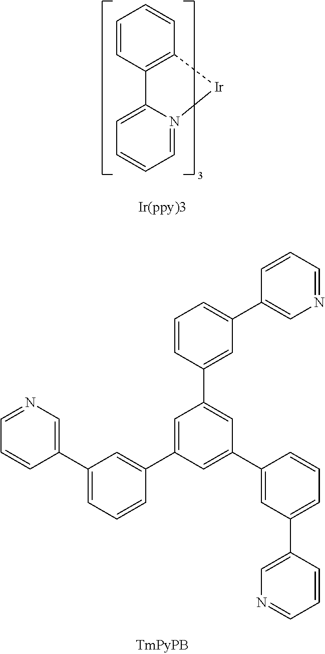

2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T₉₅ was measured by a service life measurement device (M6000) manufactured by McScience Inc., when the reference luminance was 3,500 cd/m². The results of measuring the driving voltage, light emitting efficiency, color coordinate (CIE), and service life (T₉₅) of the white organic electroluminescence device manufactured according to the present invention are shown as in Table 10.

TABLE 10

| | | | LIGHT | | SER- |
|---|---|---|---|---|---|
| | | DRIVING | EMITTING | | VICE |
| | | VOLTAGE | EFFICIENCY | CIE | LIFE |
| | COMPOUND | (V) | (cd/A) | (x, y) | (T₉₅) |
| EXAMPLE 45 | 1 | 7.47 | 67.58 | (0.214, 0.418) | 61 |
| EXAMPLE 46 | 3 | 7.42 | 69.77 | (0.213, 0.417) | 60 |
| EXAMPLE 47 | 25 | 7.38 | 68.68 | (0.213, 0.420) | 60 |
| EXAMPLE 48 | 27 | 7.36 | 69.12 | (0.215, 0.420) | 58 |
| EXAMPLE 49 | 72 | 7.42 | 69.15 | (0.215, 0.420) | 63 |
| EXAMPLE 50 | 79 | 7.46 | 68.88 | (0.214, 0.420) | 62 |
| EXAMPLE 51 | 92 | 7.49 | 69.02 | (0.214, 0.418) | 59 |
| EXAMPLE 52 | 102 | 7.45 | 68.53 | (0.215, 0.419) | 59 |
| EXAMPLE 53 | 115 | 7.45 | 70.03 | (0.216, 0,424) | 62 |
| EXAMPLE 54 | 137 | 7.42 | 69.87 | (0.214, 0.425) | 60 |
| EXAMPLE 55 | 175 | 7.41 | 68.13 | (0.219, 0.426) | 60 |
| EXAMPLE 56 | 218 | 7.40 | 69.00 | (0.218, 0.421) | 57 |
| EXAMPLE 57 | 226 | 7.39 | 69.50 | (0.215, 0.417) | 58 |
| EXAMPLE 58 | 239 | 7.48 | 67.72 | (0.209, 0.422) | 58 |
| EXAMPLE 59 | 245 | 7.49 | 68.58 | (0.210, 0.428) | 62 |
| EXAMPLE 60 | 281 | 7.46 | 69.13 | (0.209, 0.428) | 63 |
| EXAMPLE 61 | 300 | 7.42 | 68.43 | (0.210, 0.430) | 64 |
| EXAMPLE 62 | 302 | 7.41 | 69.84 | (0.212, 0.428) | 64 |
| EXAMPLE 63 | 314 | 7.40 | 70.18 | (0.219, 0.426) | 61 |
| EXAMPLE 64 | 319 | 7.46 | 70.02 | (0.218, 0.424) | 60 |
| EXAMPLE 65 | 324 | 7.42 | 70.42 | (0.215, 0.417) | 60 |
| EXAMPLE 66 | 334 | 7.42 | 70.25 | (0.214, 0.420) | 57 |
| EXAMPLE 67 | 381 | 7.39 | 69.39 | (0.214, 0.418) | 59 |
| EXAMPLE 68 | 396 | 7.40 | 69.88 | (0.214, 0.418) | 60 |
| EXAMPLE 69 | 401 | 7.48 | 69.98 | (0.215, 0.419) | 62 |
| EXAMPLE 70 | 501 | 7.47 | 68.80 | (0.219, 0.426) | 62 |
| EXAMPLE 71 | 519 | 7.42 | 69.22 | (0.220, 0.424) | 64 |
| EXAMPLE 72 | 521 | 7.43 | 70.13 | (0.219, 0.422) | 62 |
| EXAMPLE 73 | 583 | 7.45 | 69.87 | (0.209, 0.422) | 57 |
| EXAMPLE 74 | 596 | 7.41 | 68.13 | (0.210, 0.428) | 59 |
| EXAMPLE 75 | 598 | 7.38 | 69.00 | (0.209, 0.428) | 60 |
| COMPARATIVE | TmPyPB | 8.20 | 57.71 | (0.211, 0.430) | 60 |
| EXAMPLE 5 | | | | | |
| COMPARATIVE | COMPARATIVE | 7.87 | 65.84 | (0.218, 0.421) | 30 |
| EXAMPLE 6 | COMPOUND A | | | | |
| COMPARATIVE | COMPARATIVE | 8.00 | 59.21 | (0.209, 0.432) | 21 |
| EXAMPLE 7 | COMPOUND B | | | | |

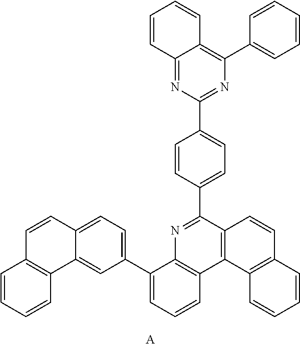

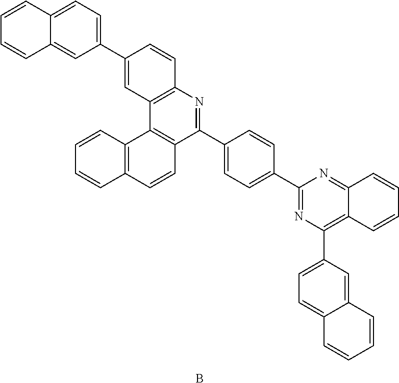

As can be seen from the results in Table 10, the organic electroluminescence device using a charge generation layer material of the 2-stack white organic electroluminescence device of the present invention has a low driving voltage and improved service life and light emitting efficiency compared to Comparative Examples 5 to 8. These results are due to having a backbone with suitable length and strength, and consisting of a suitable heterocompound structure that can be bonded to metals. Due to these structural features, when the compound used as the N-type charge generation layer is doped with a metal, a gap state is stably formed in the N-type charge generation layer, and electrons generated from the P-type charge generation layer are easily injected into the electron transport layer through the gap state generated in the N-type charge generation layer.

Therefore, it is confirmed that the P-type charge generation layer enables electrons to be well introduced into and transferred to the N-type charge generation layer, and thus the driving voltage of the organic light emitting device was lowered and the efficiency and service life were improved.

1. A heterocyclic compound represented by the following Chemical Formula 1: 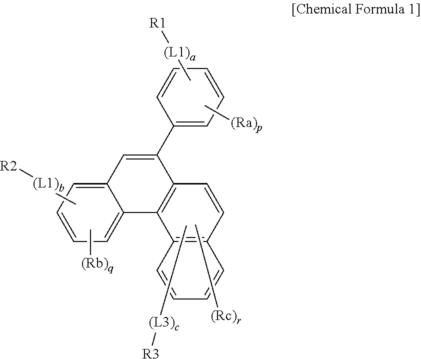
wherein, in Chemical Formula 1,
L1 to L3 are the same as or different from each other, and are each independently a direct bond; a substituted or unsubstituted C6 to C60 arylene group; or a substituted or unsubstituted C2 to C60 heteroarylene group,
R1 to R3 are the same as or different from each other, and are each independently hydrogen; deuterium; a substituted or unsubstituted amine group; a substituted or unsubstituted C6 to C60 aryl group; a substituted or unsubstituted C2 to C60 heteroaryl group; or —P(═O)RR′,
at least one of R1 to R3 is a substituted or unsubstituted amine group, and another one of R1 to R3 is a substituted or unsubstituted C2 to C60 heteroaryl group; or —P(═O)RR′,
R and R′ are the same as or different from each other, and are each independently hydrogen; deuterium; —CN; a substituted or unsubstituted C6 to C60 aryl group; or a substituted or unsubstituted C2 to C60 heteroaryl group,
Ra, Rb and Rc are the same as or different from each other, and are each independently hydrogen; deuterium; a halogen group; —CN; a substituted or unsubstituted C1 to C60 alkyl group; a substituted or unsubstituted C6 to C60 aryl group; or a substituted or unsubstituted C2 to C60 heteroaryl group,
a, b and c are an integer from 0 to 4, and when a, b and c are 2 or higher, substituents in the parenthesis are the same as or different from each other,
p is an integer from 0 to 4, and when p is 2 or higher, substituents in the parenthesis are the same as or different from each other,
q is an integer from 0 to 3, and when q is 2 or higher, substituents in the parenthesis are the same as or different from each other, and
r is an integer from 0 to 5, and when r is 2 or higher, substituents in the parenthesis are the same as or different from each other.

2. The heterocyclic compound of claim 1, wherein Chemical Formula 1 is represented by any one of the following Chemical Formulae 1-1 to 1-6: 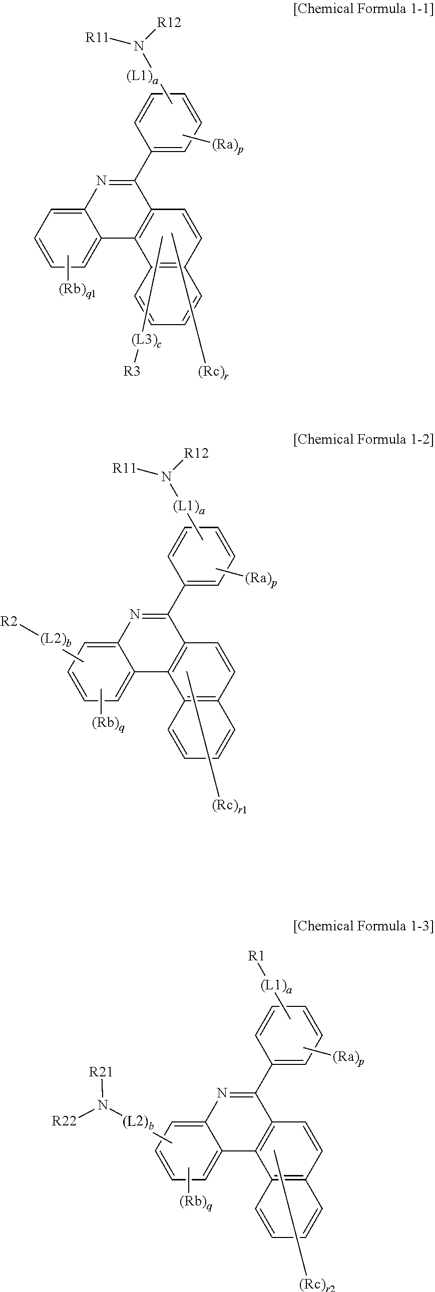 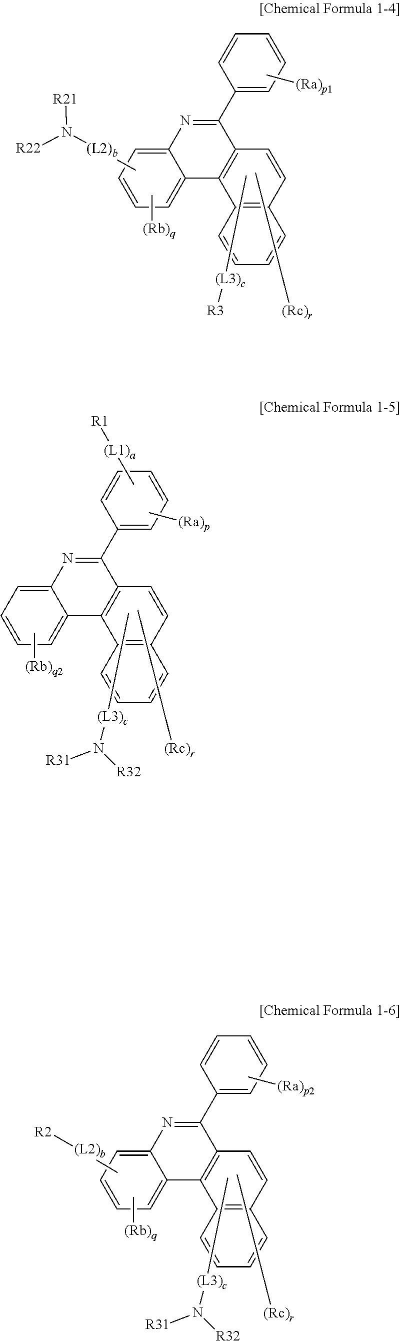
in Chemical Formulae 1-1 to 1-6,
L1 to L3, R1 to R3, Ra, Rb, Rc, a, b, c, p, q and r are the same as the definitions in Chemical Formula 1,
R11, R12, R21, R22, R31 and R32 are the same as or different from each other, and are each independently a substituted or unsubstituted C6 to C60 aryl group; or a substituted or unsubstituted C2 to C60 heteroaryl group,
p1 and p2 are each an integer from 0 to 5, and p1 and p2 are 2 or higher, substituents in the parenthesis are the same as or different from each other,
q1 and q2 are each an integer from 0 to 4, and when q1 and q2 are 2 or higher, substituents in the parenthesis are the same as or different from each other, and
r1 and r2 are each an integer from 0 to 6, and when r1 and r2 are 2 or higher, substituents in the parenthesis are the same as or different from each other.

3. The heterocyclic compound of claim 1, wherein R1 to R3 are the same as or different from each other, and are each independently hydrogen; deuterium; an amine group which is unsubstituted or substituted with a substituted or unsubstituted C6 to C40 aryl group or a substituted or unsubstituted C2 to C40 heteroaryl group; a substituted or unsubstituted C2 to C40 heteroaryl group; or —P(═O)RR′, and
any one of R1 to R3 is an amine group which is unsubstituted or substituted with a substituted or unsubstituted C6 to C40 aryl group or a substituted or unsubstituted C2 to C40 heteroaryl group, and another one of R1 to R3 is a substituted or unsubstituted C2 to C40 heteroaryl group; or —P(═O)RR′.

4. The heterocyclic compound of claim 2, wherein R11, R12, R21, R22, R31 and R32 are the same as or different from each other, and are each independently a substituted or unsubstituted C6 to C20 aryl group; or a substituted or unsubstituted C2 to C20 heteroaryl group.

5. The heterocyclic compound of claim 1, wherein L1 to L3 are the same as or different from each other, and are each independently a direct bond; or a substituted or unsubstituted C6 to C20 arylene group.

6. The heterocyclic compound of claim 1, wherein Ra, Rb and Rc are the same as or different from each other, and are each independently hydrogen; or deuterium.

7. The heterocyclic compound of claim 1, wherein a deuterium content of the heterocyclic compound of Chemical Formula 1 is 0%, or 30% to 100%.

8. The heterocyclic compound of claim 1, wherein Chemical Formula 1 is represented by any one of the following compounds:

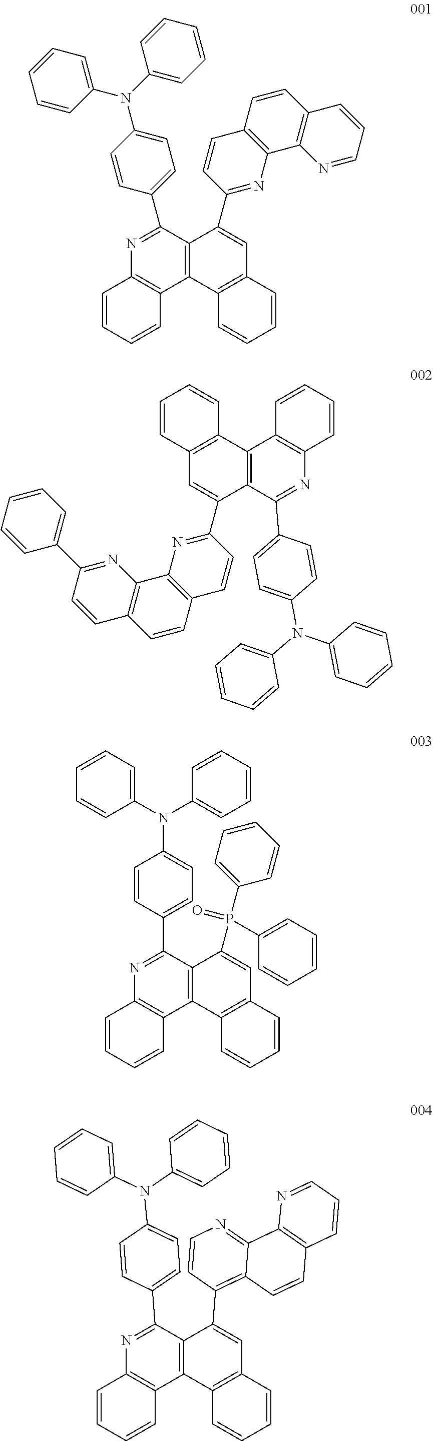 [SMILES: c1ccc(N(c2ccccc2)c2ccc(-c3nc4ccccc4c4c3c(-c3ccc5ccc6cccnc6c5n3)cc3ccccc34)cc2)cc1 ; c1ccc(-c2ccc3ccc4ccc(-c5cc6ccccc6c6c5c(-c5ccc(N(c7ccccc7)c7ccccc7)cc5)nc5ccccc56)nc4c3n2)cc1 ; O=P(c1ccccc1)(c1ccccc1)c1cc2ccccc2c2c1c(-c1ccc(N(c3ccccc3)c3ccccc3)cc1)nc1ccccc12 ; c1ccc(N(c2ccccc2)c2ccc(-c3nc4ccccc4c4c3c(-c3ccnc5c3ccc3cccnc35)cc3ccccc34)cc2)cc1]

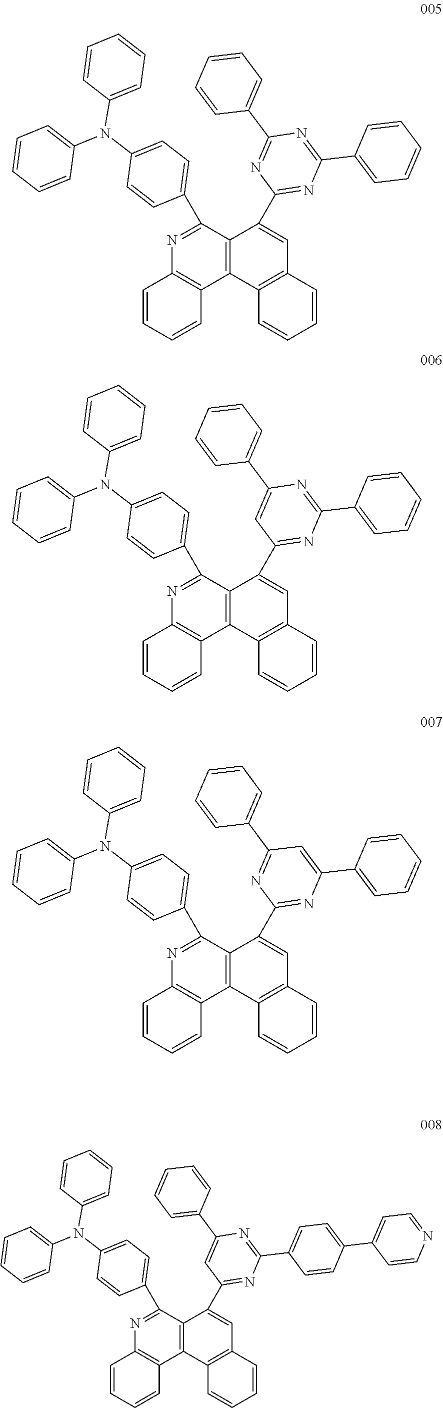 [SMILES: c1ccc(-c2nc(-c3ccccc3)nc(-c3cc4ccccc4c4c3c(-c3ccc(N(c5ccccc5)c5ccccc5)cc3)nc3ccccc34)n2)cc1 ; c1ccc(-c2cc(-c3cc4ccccc4c4c3c(-c3ccc(N(c5ccccc5)c5ccccc5)cc3)nc3ccccc34)nc(-c3ccccc3)n2)cc1 ; c1ccc(-c2cc(-c3ccccc3)nc(-c3cc4ccccc4c4c3c(-c3ccc(N(c5ccccc5)c5ccccc5)cc3)nc3ccccc34)n2)cc1 ; c1ccc(-c2cc(-c3cc4ccccc4c4c3c(-c3ccc(N(c5ccccc5)c5ccccc5)cc3)nc3ccccc34)nc(-c3ccc(-c4ccncc4)cc3)n2)cc1]

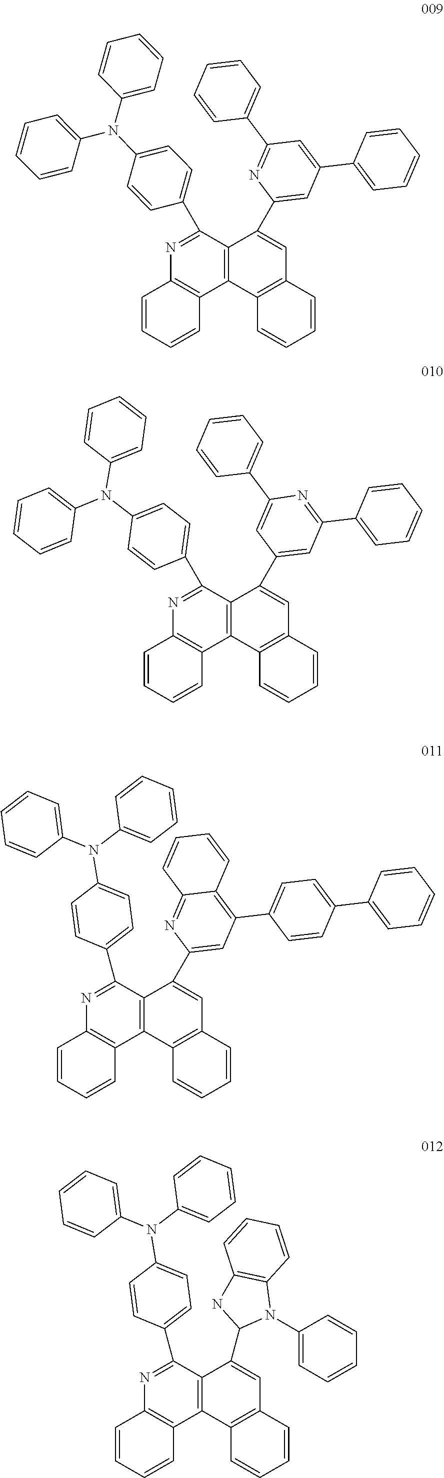 [SMILES: c1ccc(-c2cc(-c3ccccc3)nc(-c3cc4ccccc4c4c3c(-c3ccc(N(c5ccccc5)c5ccccc5)cc3)nc3ccccc34)c2)cc1 ; c1ccc(-c2cc(-c3cc4ccccc4c4c3c(-c3ccc(N(c5ccccc5)c5ccccc5)cc3)nc3ccccc34)cc(-c3ccccc3)n2)cc1 ; c1ccc(-c2ccc(-c3cc(-c4cc5ccccc5c5c4c(-c4ccc(N(c6ccccc6)c6ccccc6)cc4)nc4ccccc45)nc4ccccc34)cc2)cc1 ; c1ccc(N(c2ccccc2)c2ccc(-c3nc4ccccc4c4c3c(C3Nc5ccccc5N3c3ccccc3)cc3ccccc34)cc2)cc1]

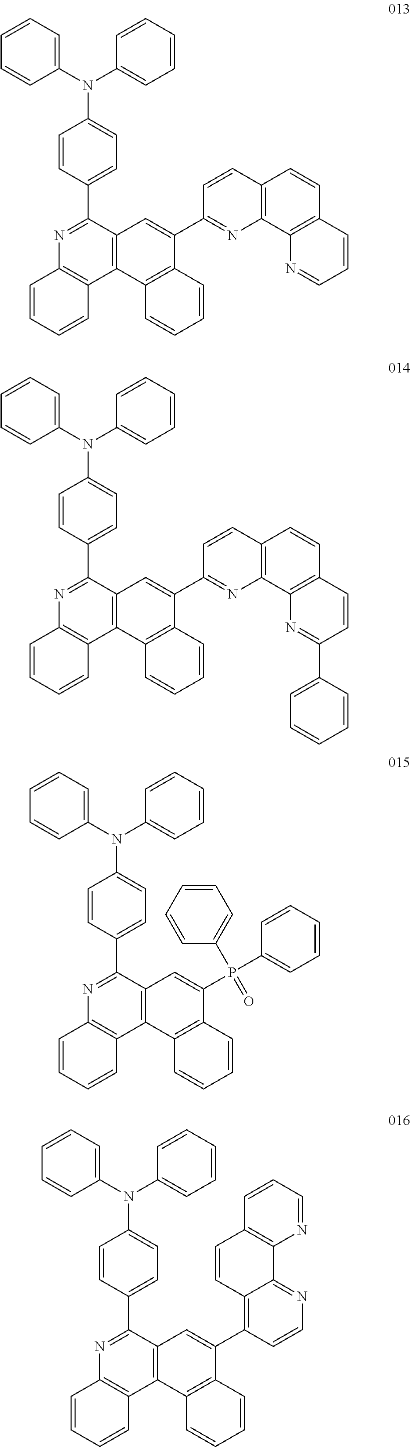

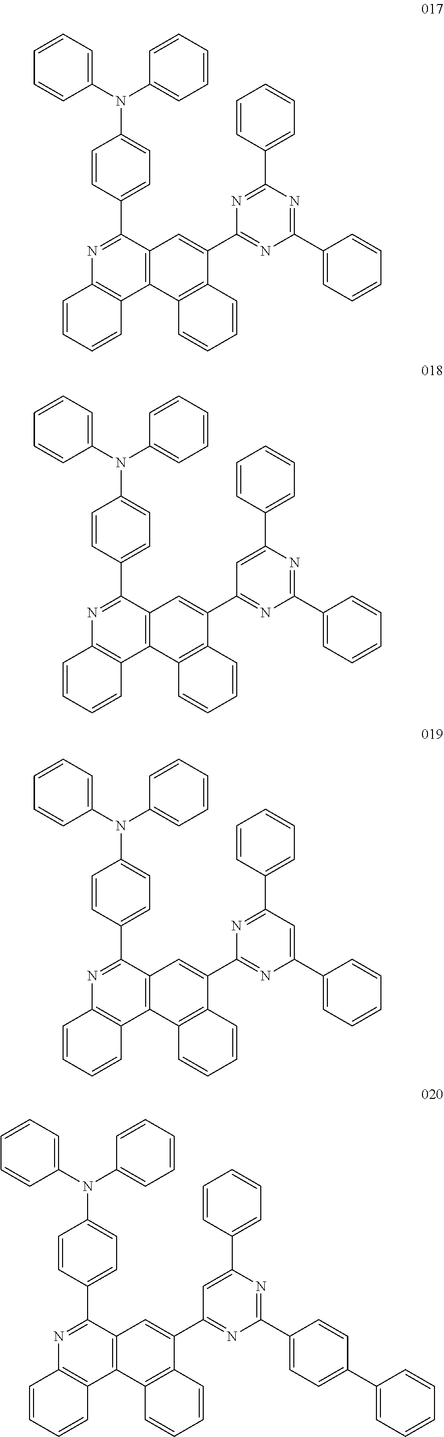 [SMILES: c1ccc(-c2nc(-c3ccccc3)nc(-c3cc4c(-c5ccc(N(c6ccccc6)c6ccccc6)cc5)nc5ccccc5c4c4ccccc34)n2)cc1 ; c1ccc(-c2cc(-c3cc4c(-c5ccc(N(c6ccccc6)c6ccccc6)cc5)nc5ccccc5c4c4ccccc34)nc(-c3ccccc3)n2)cc1 ; c1ccc(-c2cc(-c3ccccc3)nc(-c3cc4c(-c5ccc(N(c6ccccc6)c6ccccc6)cc5)nc5ccccc5c4c4ccccc34)n2)cc1 ; c1ccc(-c2ccc(-c3nc(-c4ccccc4)cc(-c4cc5c(-c6ccc(N(c7ccccc7)c7ccccc7)cc6)nc6ccccc6c5c5ccccc45)n3)cc2)cc1]

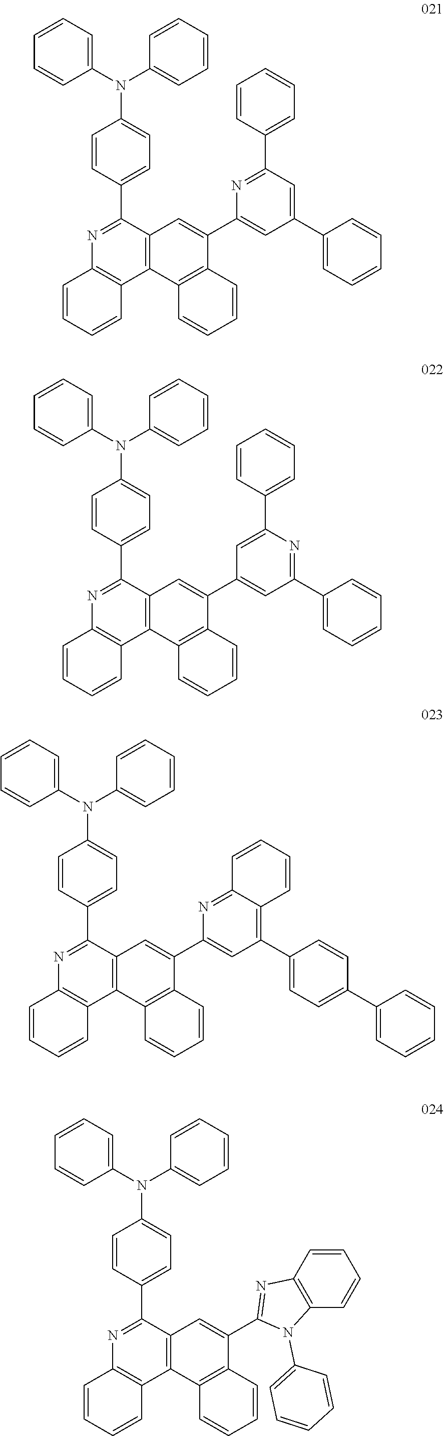 [SMILES: c1ccc(-c2cc(-c3ccccc3)nc(-c3cc4c(-c5ccc(N(c6ccccc6)c6ccccc6)cc5)nc5ccccc5c4c4ccccc34)c2)cc1 ; c1ccc(-c2cc(-c3cc4c(-c5ccc(N(c6ccccc6)c6ccccc6)cc5)nc5ccccc5c4c4ccccc34)cc(-c3ccccc3)n2)cc1 ; c1ccc(-c2ccc(-c3cc(-c4cc5c(-c6ccc(N(c7ccccc7)c7ccccc7)cc6)nc6ccccc6c5c5ccccc45)nc4ccccc34)cc2)cc1 ; c1ccc(N(c2ccccc2)c2ccc(-c3nc4ccccc4c4c3cc(-c3nc5ccccc5n3-c3ccccc3)c3ccccc34)cc2)cc1]

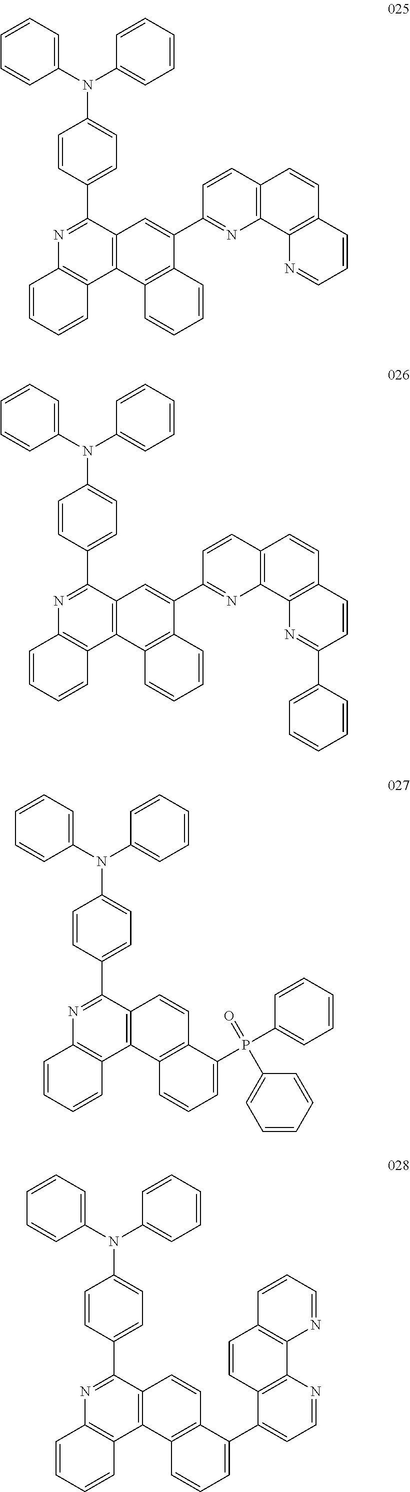 [SMILES: c1ccc(N(c2ccccc2)c2ccc(-c3nc4ccccc4c4c3cc(-c3ccc5ccc6cccnc6c5n3)c3ccccc34)cc2)cc1 ; c1ccc(-c2ccc3ccc4ccc(-c5cc6c(-c7ccc(N(c8ccccc8)c8ccccc8)cc7)nc7ccccc7c6c6ccccc56)nc4c3n2)cc1 ; O=P(c1ccccc1)(c1ccccc1)c1cccc2c1ccc1c(-c3ccc(N(c4ccccc4)c4ccccc4)cc3)nc3ccccc3c12 ; c1ccc(N(c2ccccc2)c2ccc(-c3nc4ccccc4c4c3ccc3c(-c5ccnc6c5ccc5cccnc56)cccc34)cc2)cc1]

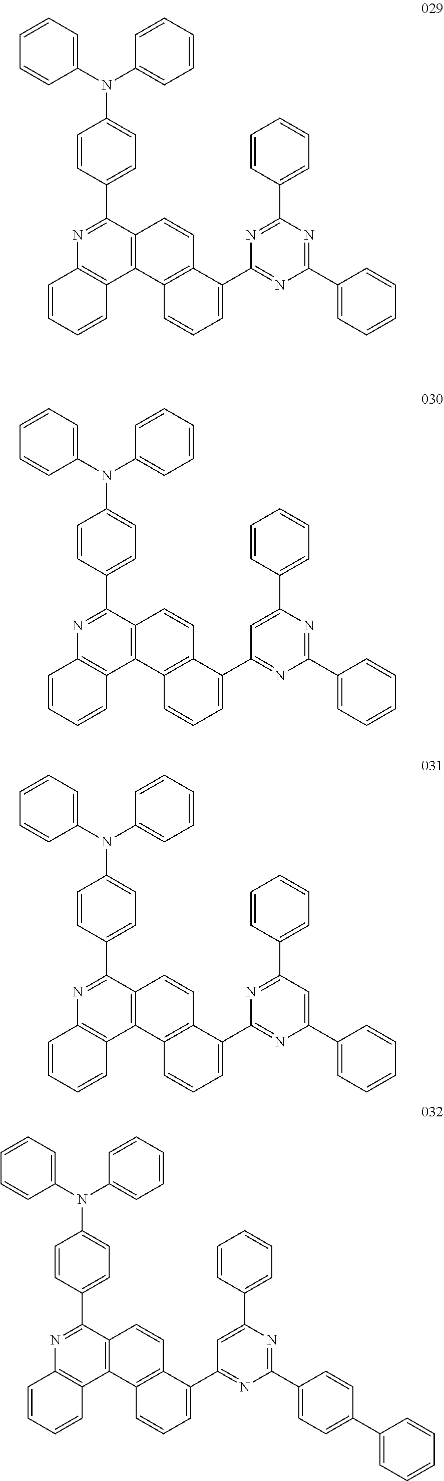 [SMILES: c1ccc(-c2nc(-c3ccccc3)nc(-c3cccc4c3ccc3c(-c5ccc(N(c6ccccc6)c6ccccc6)cc5)nc5ccccc5c34)n2)cc1 ; c1ccc(-c2cc(-c3cccc4c3ccc3c(-c5ccc(N(c6ccccc6)c6ccccc6)cc5)nc5ccccc5c34)nc(-c3ccccc3)n2)cc1 ; c1ccc(-c2cc(-c3ccccc3)nc(-c3cccc4c3ccc3c(-c5ccc(N(c6ccccc6)c6ccccc6)cc5)nc5ccccc5c34)n2)cc1 ; c1ccc(-c2ccc(-c3nc(-c4ccccc4)cc(-c4cccc5c4ccc4c(-c6ccc(N(c7ccccc7)c7ccccc7)cc6)nc6ccccc6c45)n3)cc2)cc1]

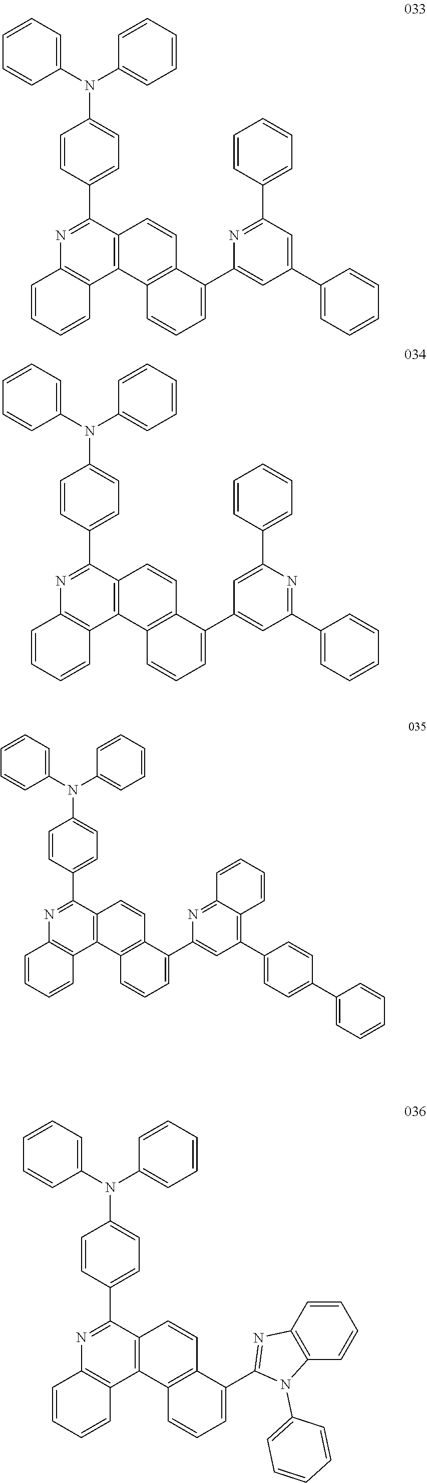

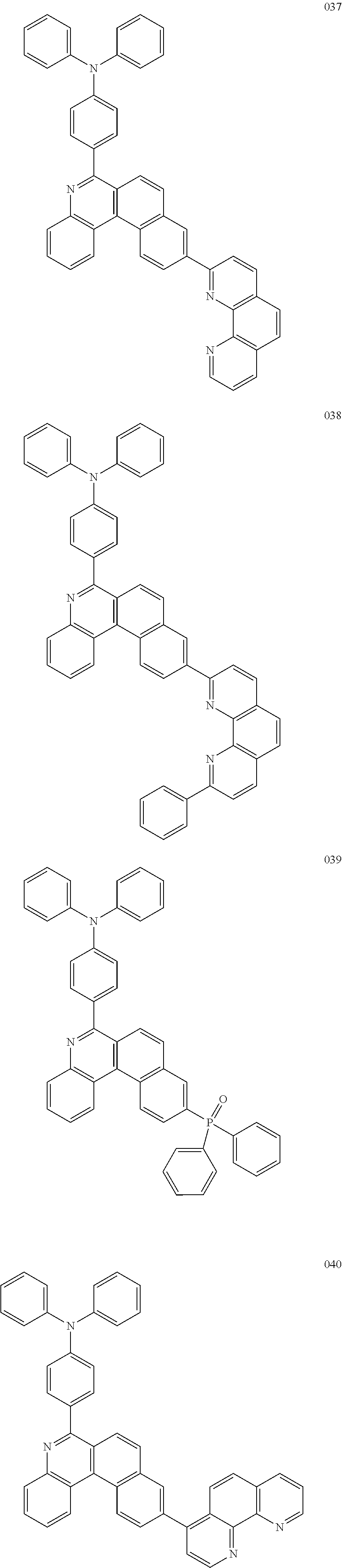

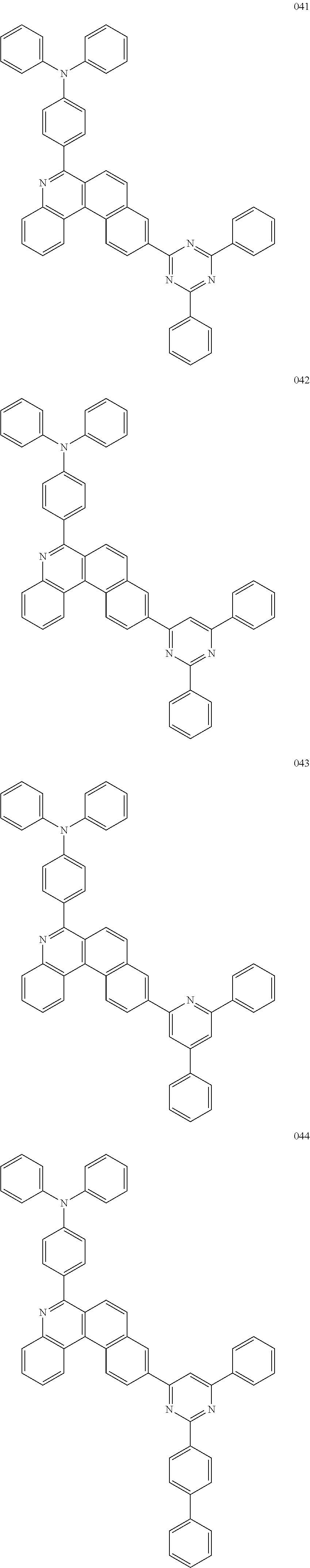 [SMILES: c1ccc(-c2nc(-c3ccccc3)nc(-c3ccc4c(ccc5c(-c6ccc(N(c7ccccc7)c7ccccc7)cc6)nc6ccccc6c54)c3)n2)cc1 ; c1ccc(-c2cc(-c3ccc4c(ccc5c(-c6ccc(N(c7ccccc7)c7ccccc7)cc6)nc6ccccc6c54)c3)nc(-c3ccccc3)n2)cc1 ; c1ccc(-c2cc(-c3ccccc3)nc(-c3ccc4c(ccc5c(-c6ccc(N(c7ccccc7)c7ccccc7)cc6)nc6ccccc6c54)c3)c2)cc1 ; c1ccc(-c2ccc(-c3nc(-c4ccccc4)cc(-c4ccc5c(ccc6c(-c7ccc(N(c8ccccc8)c8ccccc8)cc7)nc7ccccc7c65)c4)n3)cc2)cc1]

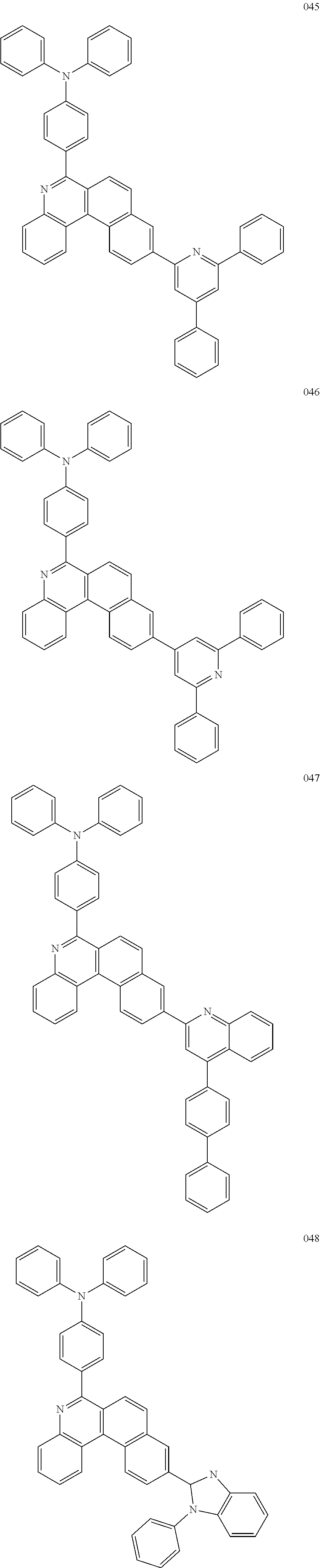 [SMILES: c1ccc(-c2cc(-c3ccccc3)nc(-c3ccc4c(ccc5c(-c6ccc(N(c7ccccc7)c7ccccc7)cc6)nc6ccccc6c54)c3)c2)cc1 ; c1ccc(-c2cc(-c3ccc4c(ccc5c(-c6ccc(N(c7ccccc7)c7ccccc7)cc6)nc6ccccc6c54)c3)cc(-c3ccccc3)n2)cc1 ; c1ccc(-c2ccc(-c3cc(-c4ccc5c(ccc6c(-c7ccc(N(c8ccccc8)c8ccccc8)cc7)nc7ccccc7c65)c4)nc4ccccc34)cc2)cc1 ; c1ccc(N(c2ccccc2)c2ccc(-c3nc4ccccc4c4c3ccc3cc(C5Nc6ccccc6N5c5ccccc5)ccc34)cc2)cc1]

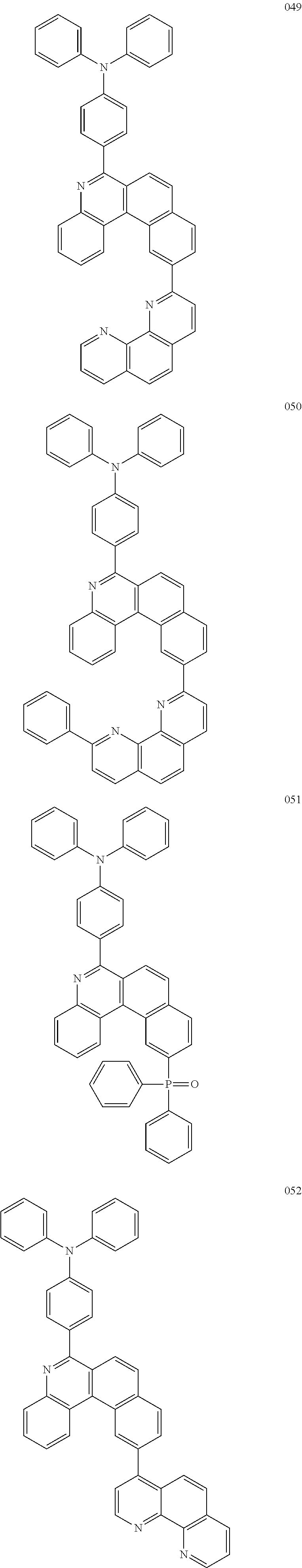

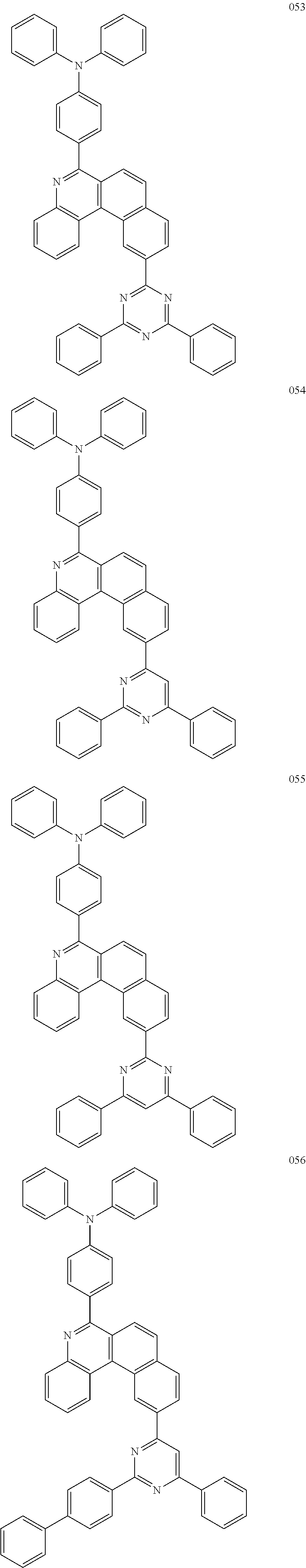

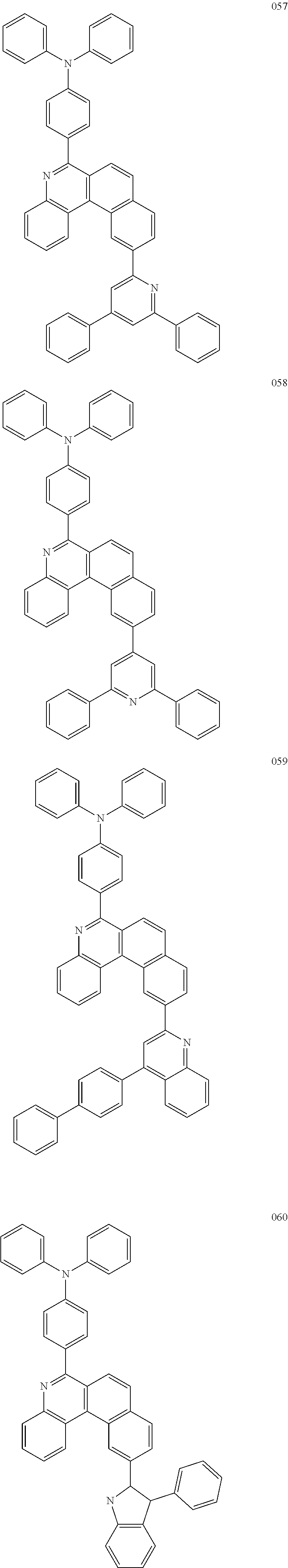

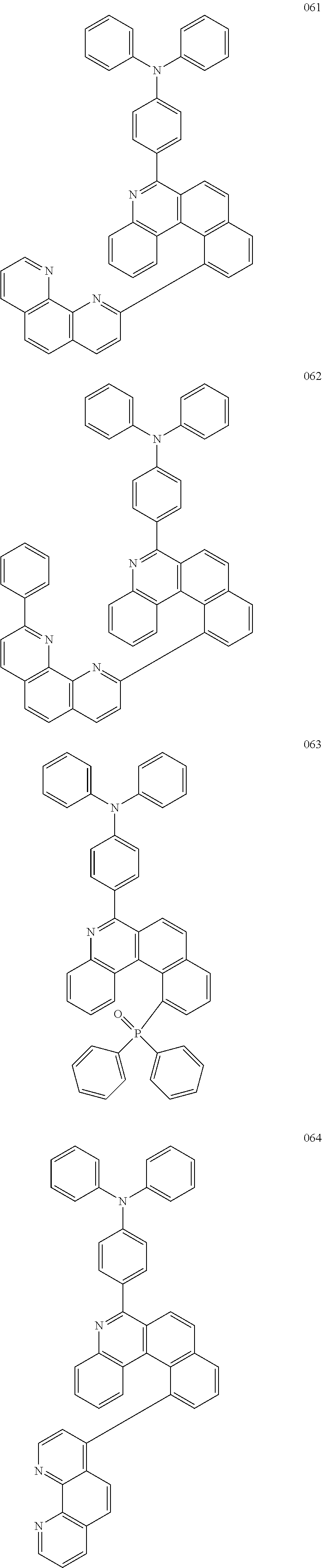

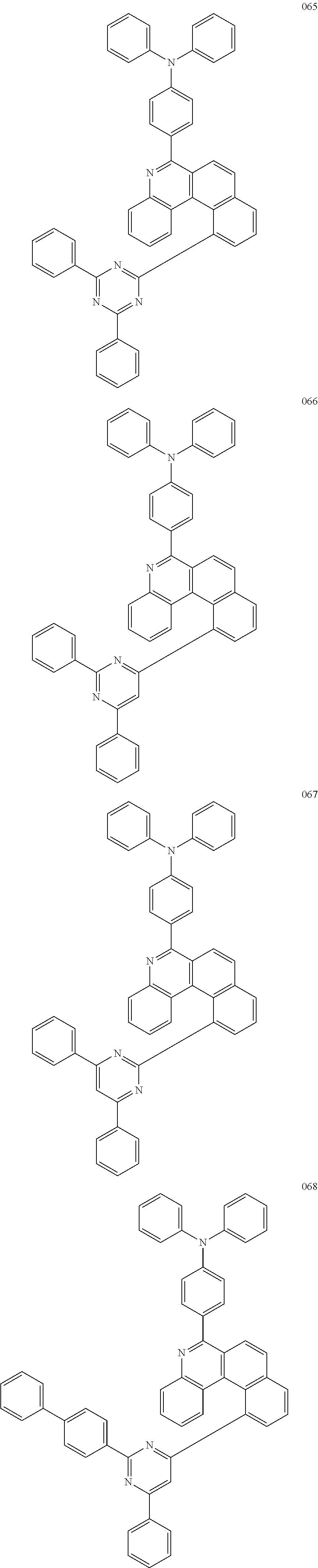

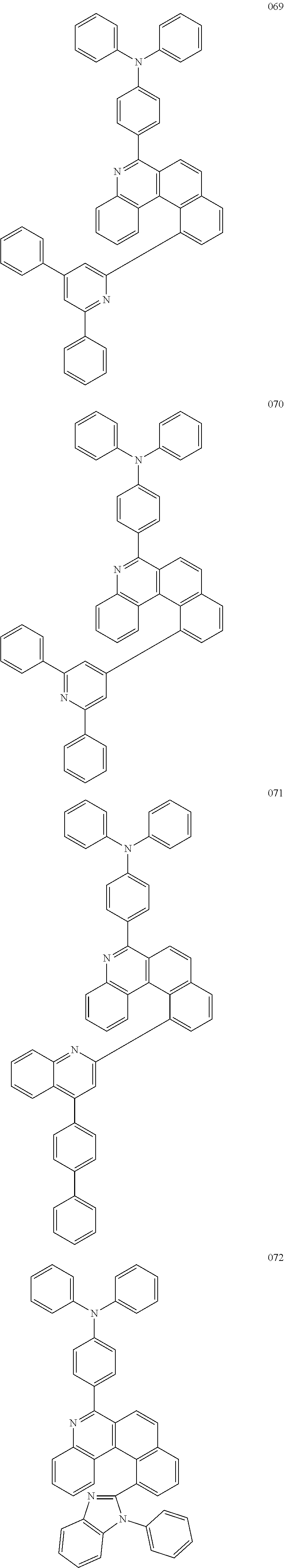

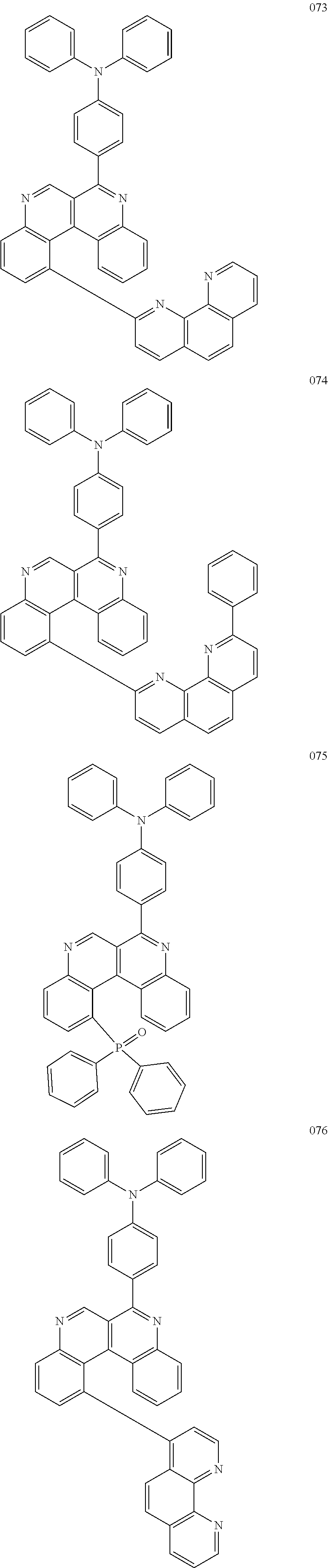

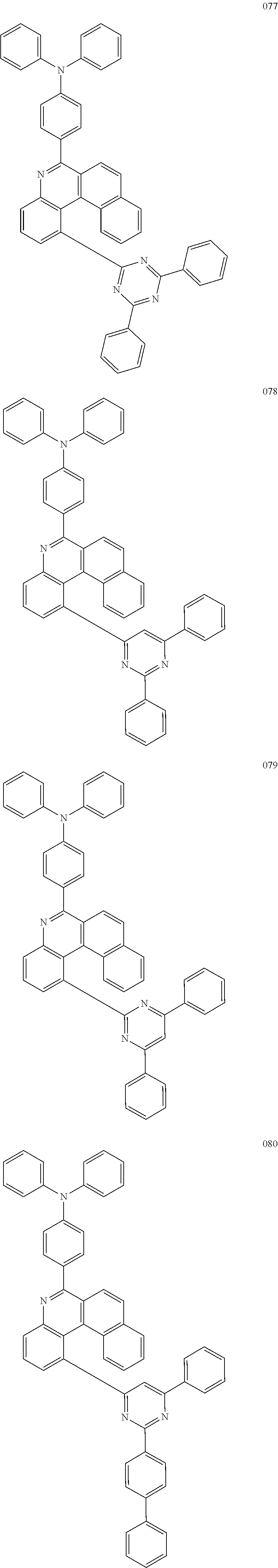

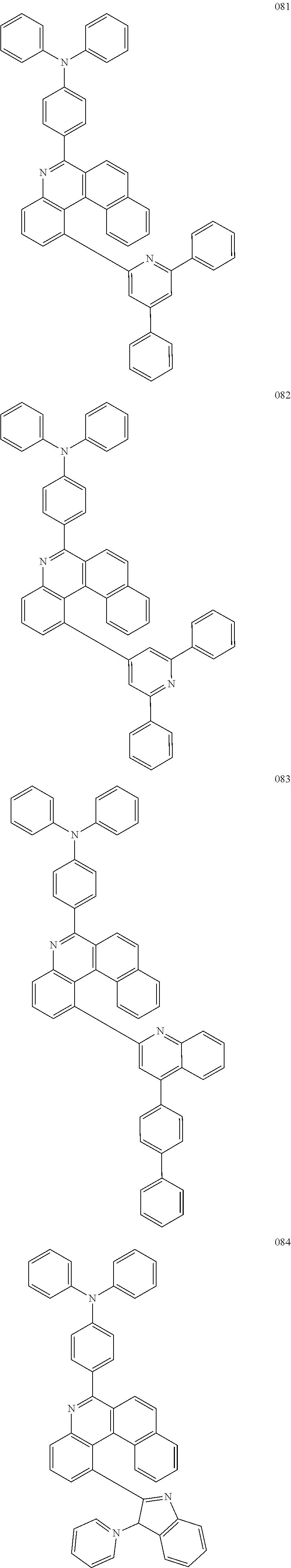

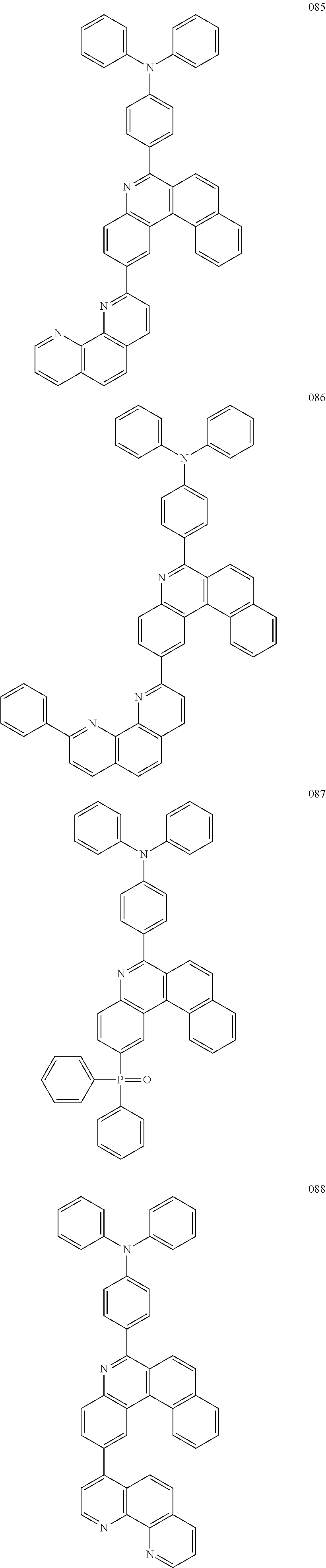

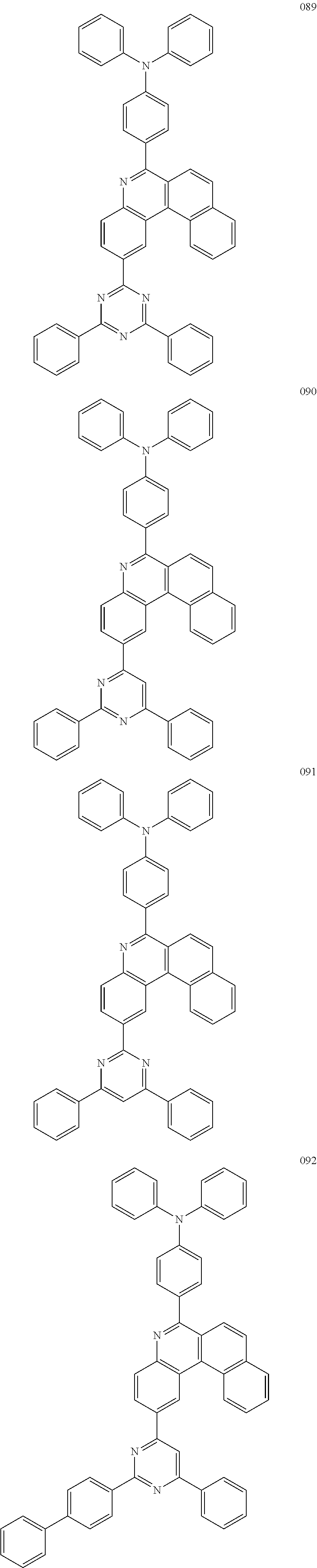

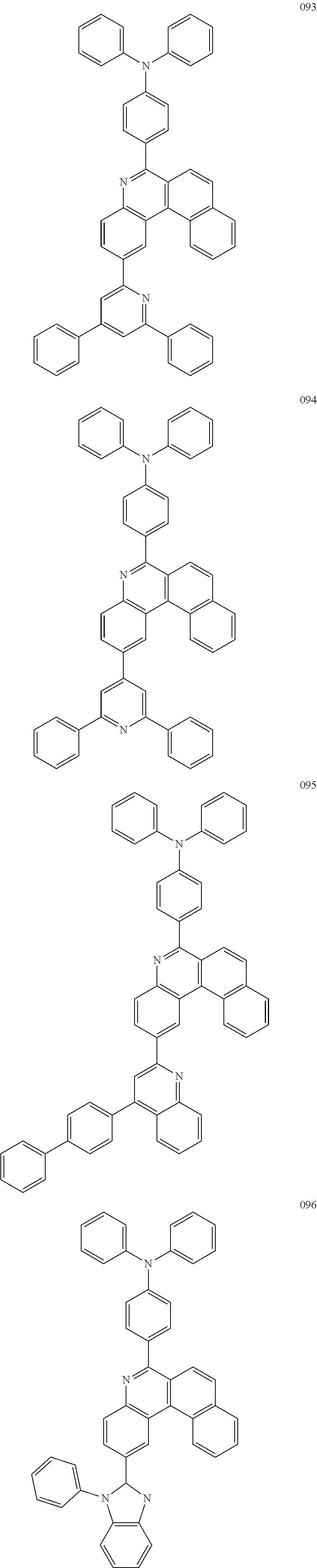

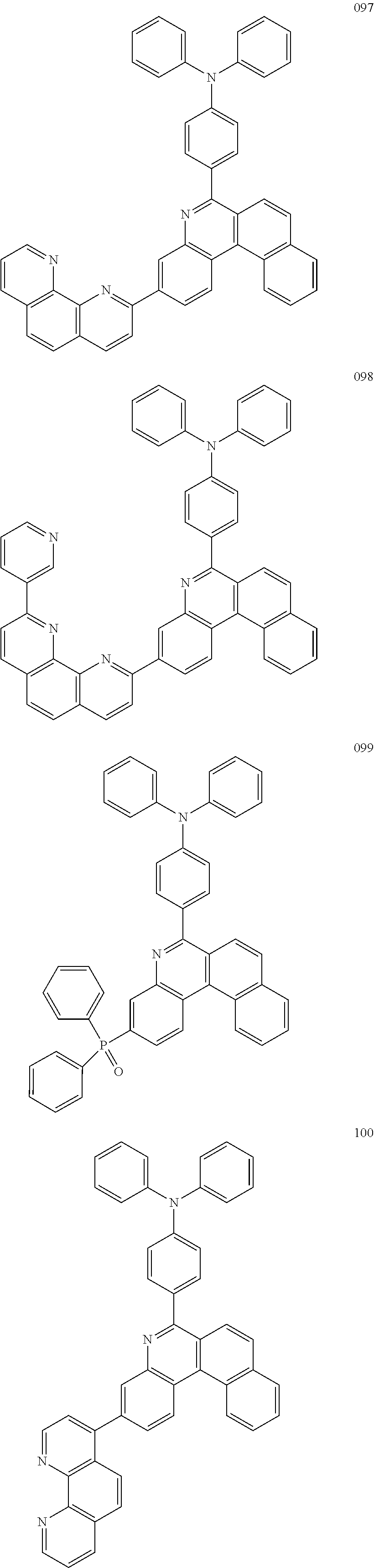 [SMILES: c1ccc(N(c2ccccc2)c2ccc(-c3nc4cc(-c5ccc6ccc7cccnc7c6n5)ccc4c4c3ccc3ccccc34)cc2)cc1 ; c1ccc(N(c2ccccc2)c2ccc(-c3nc4cc(-c5ccc6ccc7ccc(-c8cccnc8)nc7c6n5)ccc4c4c3ccc3ccccc34)cc2)cc1 ; O=P(c1ccccc1)(c1ccccc1)c1ccc2c(c1)nc(-c1ccc(N(c3ccccc3)c3ccccc3)cc1)c1ccc3ccccc3c12 ; c1ccc(N(c2ccccc2)c2ccc(-c3nc4cc(-c5ccnc6c5ccc5cccnc56)ccc4c4c3ccc3ccccc34)cc2)cc1]

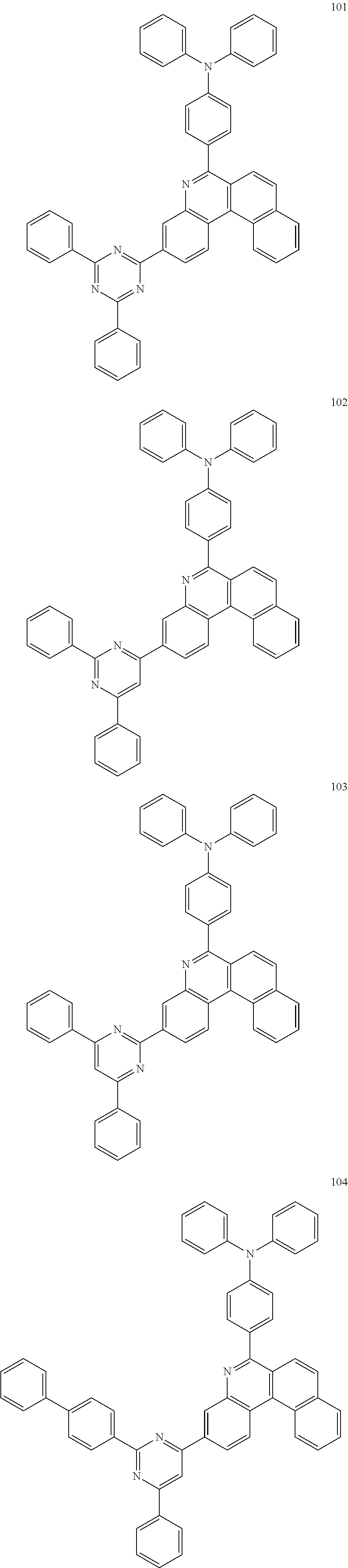 [SMILES: c1ccc(-c2nc(-c3ccccc3)nc(-c3ccc4c(c3)nc(-c3ccc(N(c5ccccc5)c5ccccc5)cc3)c3ccc5ccccc5c34)n2)cc1 ; c1ccc(-c2cc(-c3ccc4c(c3)nc(-c3ccc(N(c5ccccc5)c5ccccc5)cc3)c3ccc5ccccc5c34)nc(-c3ccccc3)n2)cc1 ; c1ccc(-c2cc(-c3ccccc3)nc(-c3ccc4c(c3)nc(-c3ccc(N(c5ccccc5)c5ccccc5)cc3)c3ccc5ccccc5c34)n2)cc1 ; c1ccc(-c2ccc(-c3nc(-c4ccccc4)cc(-c4ccc5c(c4)nc(-c4ccc(N(c6ccccc6)c6ccccc6)cc4)c4ccc6ccccc6c45)n3)cc2)cc1]

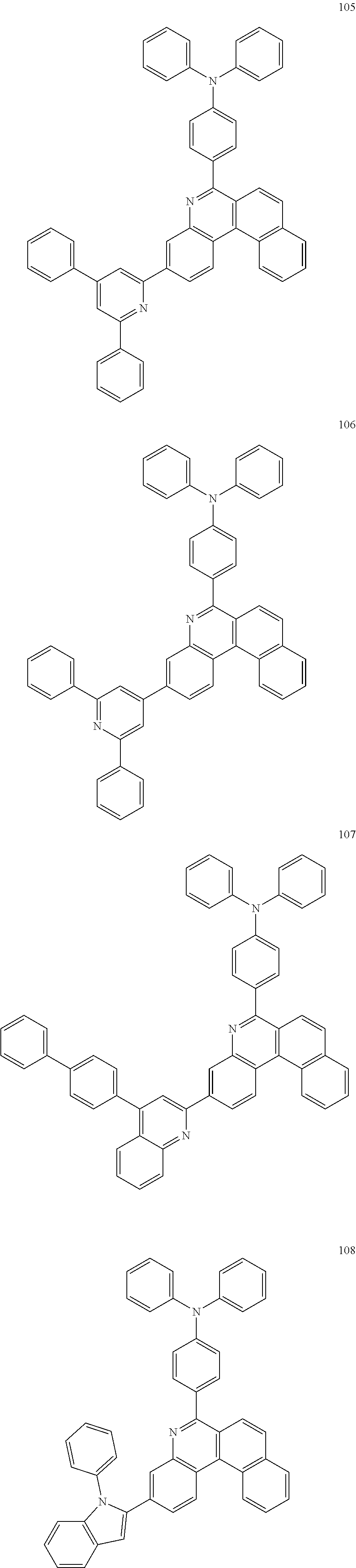 [SMILES: c1ccc(-c2cc(-c3ccccc3)nc(-c3ccc4c(c3)nc(-c3ccc(N(c5ccccc5)c5ccccc5)cc3)c3ccc5ccccc5c34)c2)cc1 ; c1ccc(-c2cc(-c3ccc4c(c3)nc(-c3ccc(N(c5ccccc5)c5ccccc5)cc3)c3ccc5ccccc5c34)cc(-c3ccccc3)n2)cc1 ; c1ccc(-c2ccc(-c3cc(-c4ccc5c(c4)nc(-c4ccc(N(c6ccccc6)c6ccccc6)cc4)c4ccc6ccccc6c45)nc4ccccc34)cc2)cc1 ; c1ccc(N(c2ccccc2)c2ccc(-c3nc4cc(-c5cc6ccccc6n5-c5ccccc5)ccc4c4c3ccc3ccccc34)cc2)cc1]

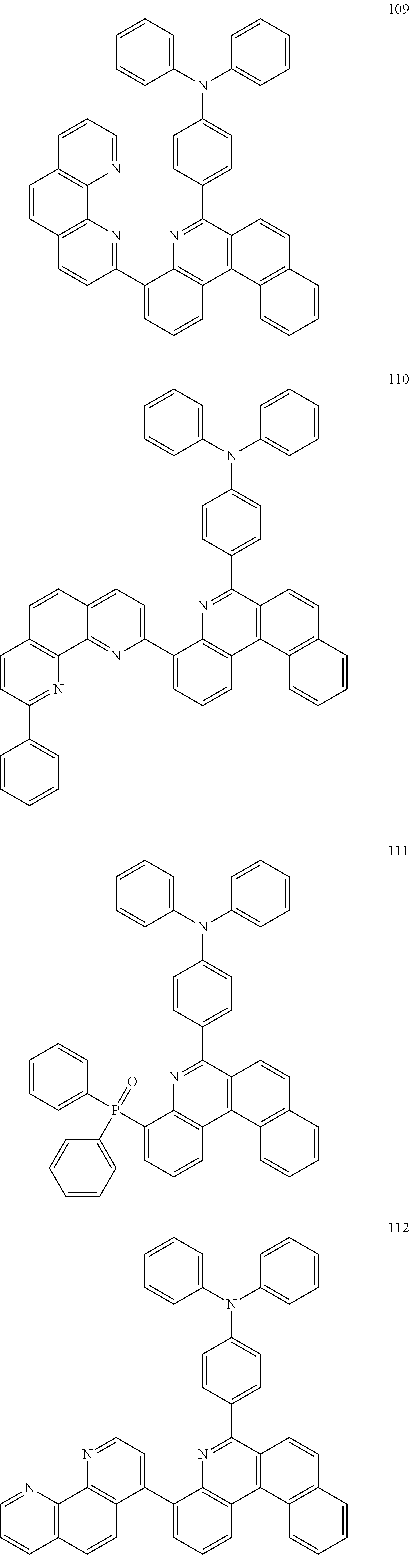 [SMILES: c1ccc(N(c2ccccc2)c2ccc(-c3nc4c(-c5ccc6ccc7cccnc7c6n5)cccc4c4c3ccc3ccccc34)cc2)cc1 ; c1ccc(-c2ccc3ccc4ccc(-c5cccc6c5nc(-c5ccc(N(c7ccccc7)c7ccccc7)cc5)c5ccc7ccccc7c56)nc4c3n2)cc1 ; O=P(c1ccccc1)(c1ccccc1)c1cccc2c1nc(-c1ccc(N(c3ccccc3)c3ccccc3)cc1)c1ccc3ccccc3c12 ; c1ccc(N(c2ccccc2)c2ccc(-c3nc4c(-c5ccnc6c5ccc5cccnc56)cccc4c4c3ccc3ccccc34)cc2)cc1]

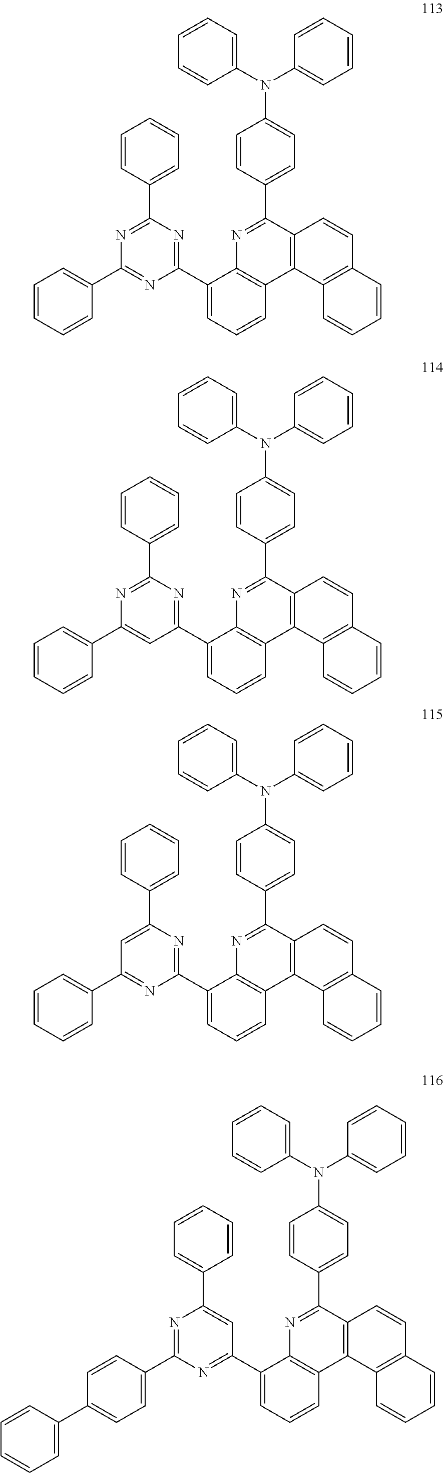

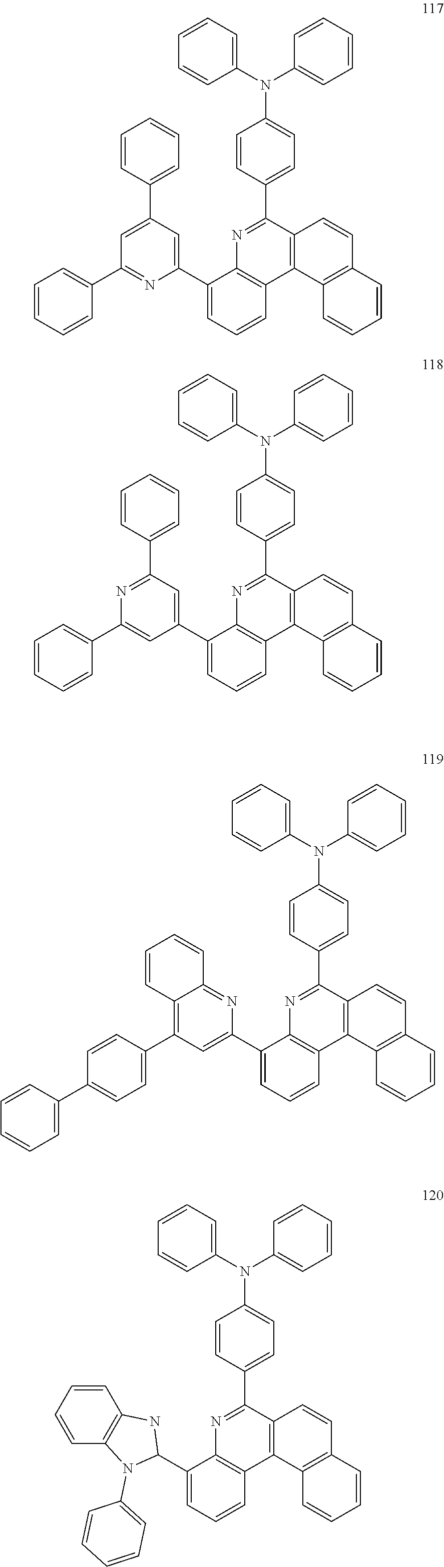 [SMILES: c1ccc(-c2cc(-c3ccccc3)nc(-c3cccc4c3nc(-c3ccc(N(c5ccccc5)c5ccccc5)cc3)c3ccc5ccccc5c34)c2)cc1 ; c1ccc(-c2cc(-c3cccc4c3nc(-c3ccc(N(c5ccccc5)c5ccccc5)cc3)c3ccc5ccccc5c34)cc(-c3ccccc3)n2)cc1 ; c1ccc(-c2ccc(-c3cc(-c4cccc5c4nc(-c4ccc(N(c6ccccc6)c6ccccc6)cc4)c4ccc6ccccc6c45)nc4ccccc34)cc2)cc1 ; c1ccc(N(c2ccccc2)c2ccc(-c3nc4c(C5Nc6ccccc6N5c5ccccc5)cccc4c4c3ccc3ccccc34)cc2)cc1]

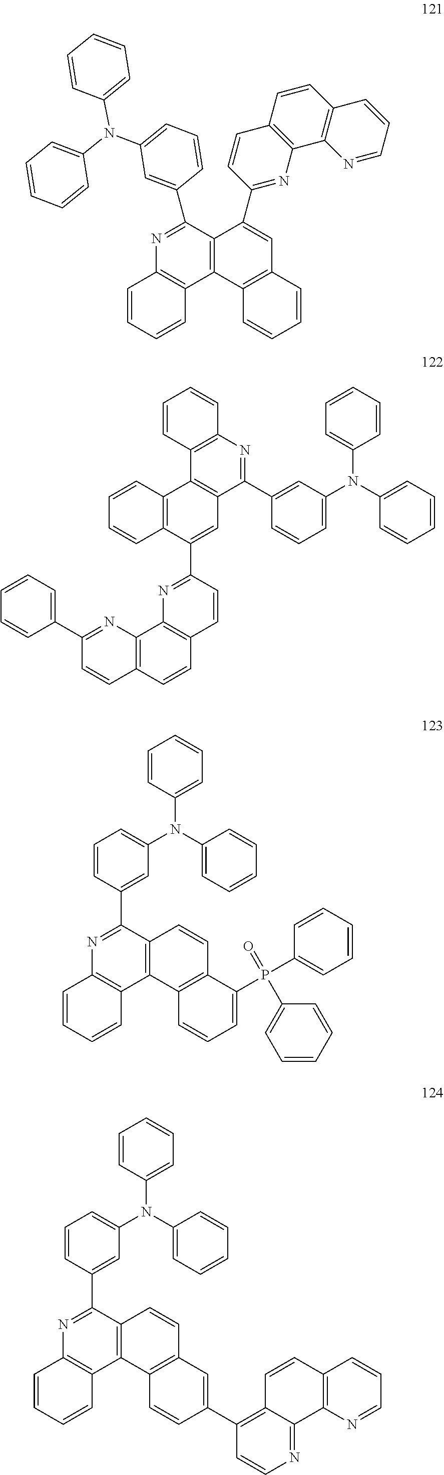 [SMILES: c1ccc(N(c2ccccc2)c2cccc(-c3nc4ccccc4c4c3c(-c3ccc5ccc6cccnc6c5n3)cc3ccccc34)c2)cc1 ; c1ccc(-c2ccc3ccc4ccc(-c5cc6c(-c7cccc(N(c8ccccc8)c8ccccc8)c7)nc7ccccc7c6c6ccccc56)nc4c3n2)cc1 ; O=P(c1ccccc1)(c1ccccc1)c1cccc2c1ccc1c(-c3cccc(N(c4ccccc4)c4ccccc4)c3)nc3ccccc3c12 ; c1ccc(N(c2ccccc2)c2cccc(-c3nc4ccccc4c4c3ccc3cc(-c5ccnc6c5ccc5cccnc56)ccc34)c2)cc1]

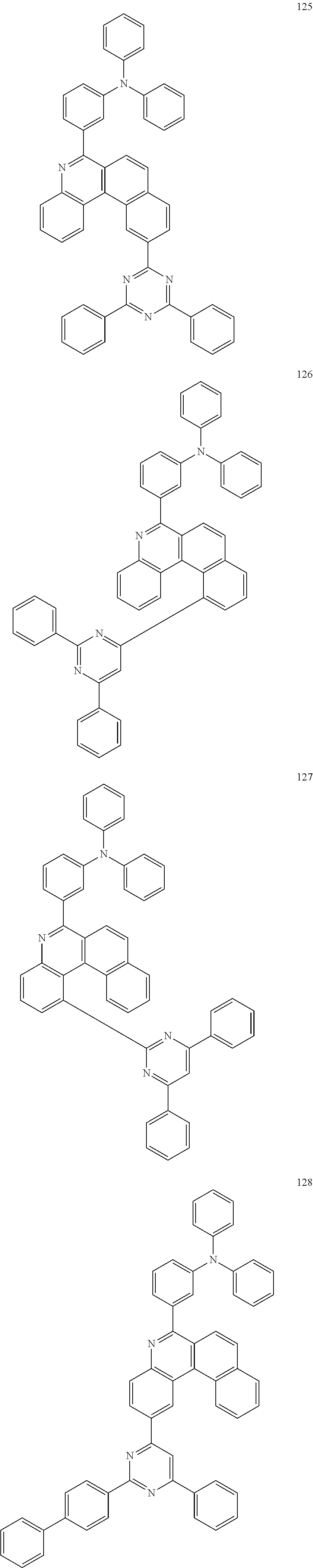

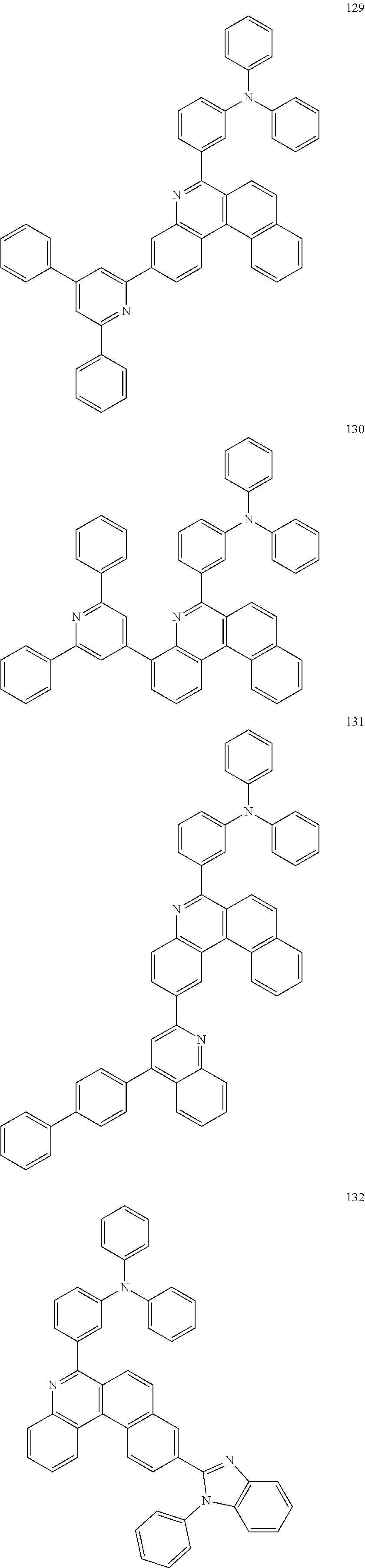

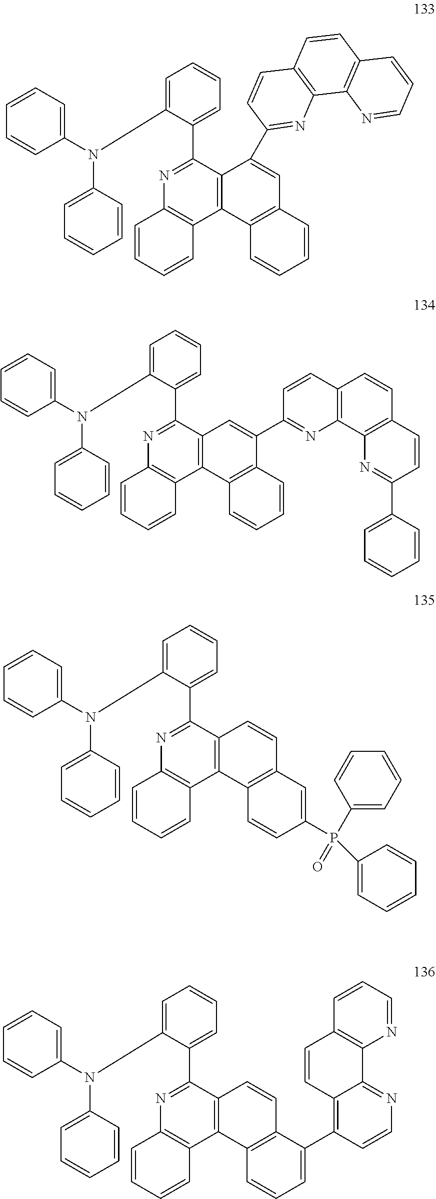 [SMILES: c1ccc(N(c2ccccc2)c2ccccc2-c2nc3ccccc3c3c2c(-c2ccc4ccc5cccnc5c4n2)cc2ccccc23)cc1 ; c1ccc(-c2ccc3ccc4ccc(-c5cc6c(-c7ccccc7N(c7ccccc7)c7ccccc7)nc7ccccc7c6c6ccccc56)nc4c3n2)cc1 ; O=P(c1ccccc1)(c1ccccc1)c1ccc2c(ccc3c(-c4ccccc4N(c4ccccc4)c4ccccc4)nc4ccccc4c32)c1 ; c1ccc(N(c2ccccc2)c2ccccc2-c2nc3ccccc3c3c2ccc2c(-c4ccnc5c4ccc4cccnc45)cccc23)cc1]

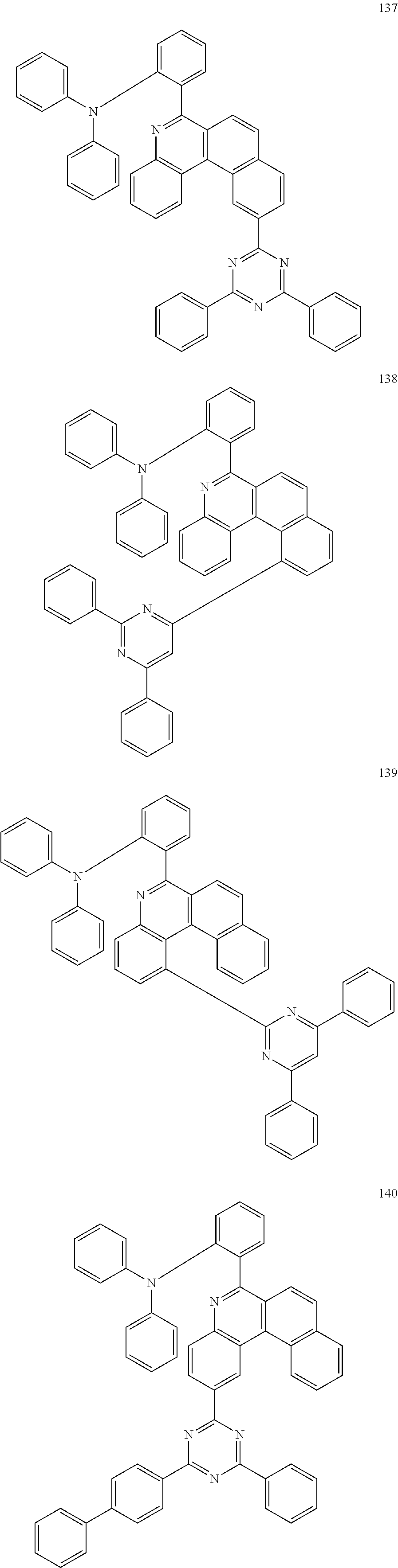 [SMILES: c1ccc(-c2nc(-c3ccccc3)nc(-c3ccc4ccc5c(-c6ccccc6N(c6ccccc6)c6ccccc6)nc6ccccc6c5c4c3)n2)cc1 ; c1ccc(-c2cc(-c3cccc4ccc5c(-c6ccccc6N(c6ccccc6)c6ccccc6)nc6ccccc6c5c34)nc(-c3ccccc3)n2)cc1 ; c1ccc(-c2cc(-c3ccccc3)nc(-c3cccc4nc(-c5ccccc5N(c5ccccc5)c5ccccc5)c5ccc6ccccc6c5c34)n2)cc1 ; c1ccc(-c2ccc(-c3nc(-c4ccccc4)nc(-c4ccc5nc(-c6ccccc6N(c6ccccc6)c6ccccc6)c6ccc7ccccc7c6c5c4)n3)cc2)cc1]

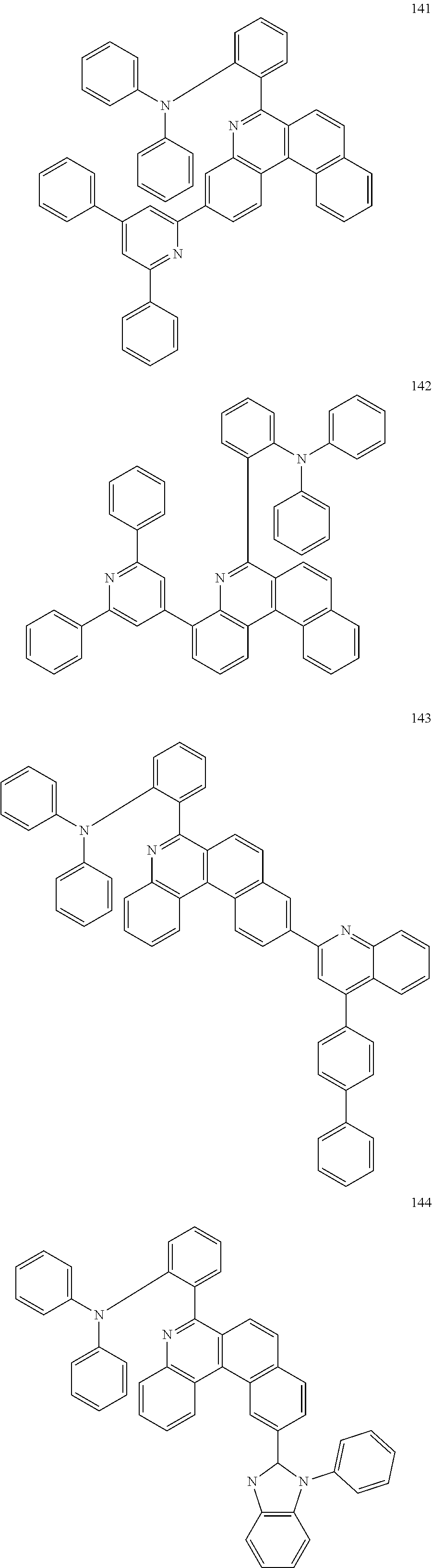 [SMILES: c1ccc(-c2cc(-c3ccccc3)nc(-c3ccc4c(c3)nc(-c3ccccc3N(c3ccccc3)c3ccccc3)c3ccc5ccccc5c34)c2)cc1 ; c1ccc(-c2cc(-c3cccc4c3nc(-c3ccccc3N(c3ccccc3)c3ccccc3)c3ccc5ccccc5c34)cc(-c3ccccc3)n2)cc1 ; c1ccc(-c2ccc(-c3cc(-c4ccc5c(ccc6c(-c7ccccc7N(c7ccccc7)c7ccccc7)nc7ccccc7c65)c4)nc4ccccc34)cc2)cc1 ; c1ccc(N(c2ccccc2)c2ccccc2-c2nc3ccccc3c3c2ccc2ccc(C4Nc5ccccc5N4c4ccccc4)cc23)cc1]

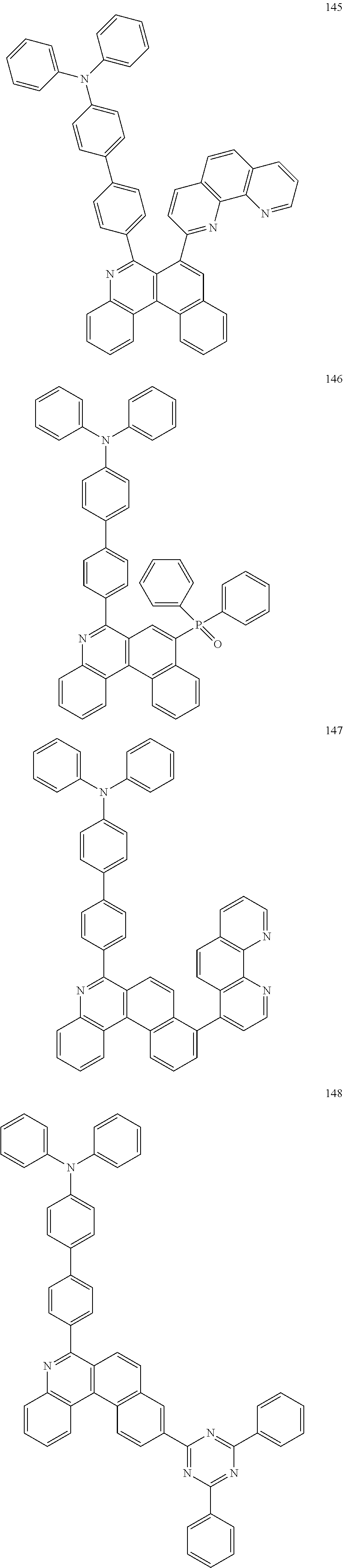

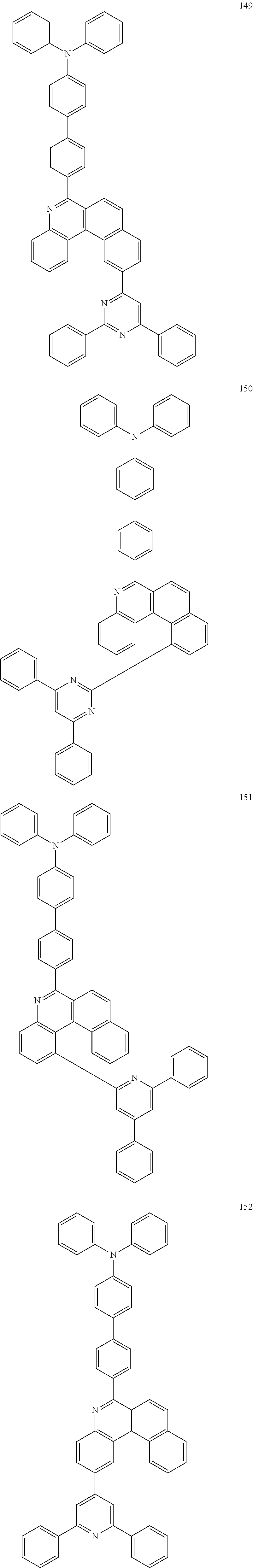

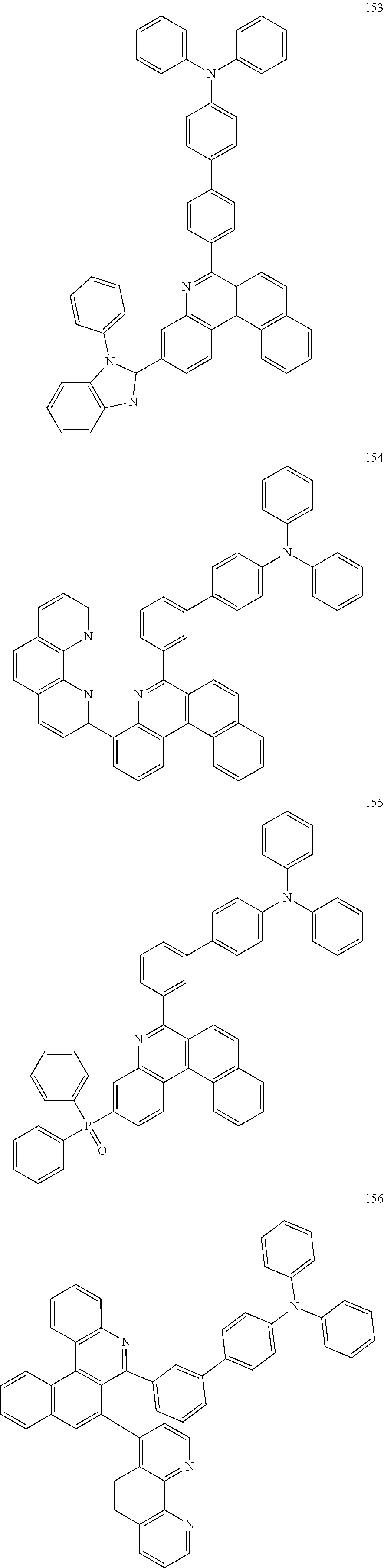 [SMILES: c1ccc(N(c2ccccc2)c2ccc(-c3ccc(-c4nc5cc(C6Nc7ccccc7N6c6ccccc6)ccc5c5c4ccc4ccccc45)cc3)cc2)cc1 ; c1ccc(N(c2ccccc2)c2ccc(-c3cccc(-c4nc5c(-c6ccc7ccc8cccnc8c7n6)cccc5c5c4ccc4ccccc45)c3)cc2)cc1 ; O=P(c1ccccc1)(c1ccccc1)c1ccc2c(c1)nc(-c1cccc(-c3ccc(N(c4ccccc4)c4ccccc4)cc3)c1)c1ccc3ccccc3c12 ; c1ccc(N(c2ccccc2)c2ccc(-c3cccc(-c4nc5ccccc5c5c4c(-c4ccnc6c4ccc4cccnc46)cc4ccccc45)c3)cc2)cc1]

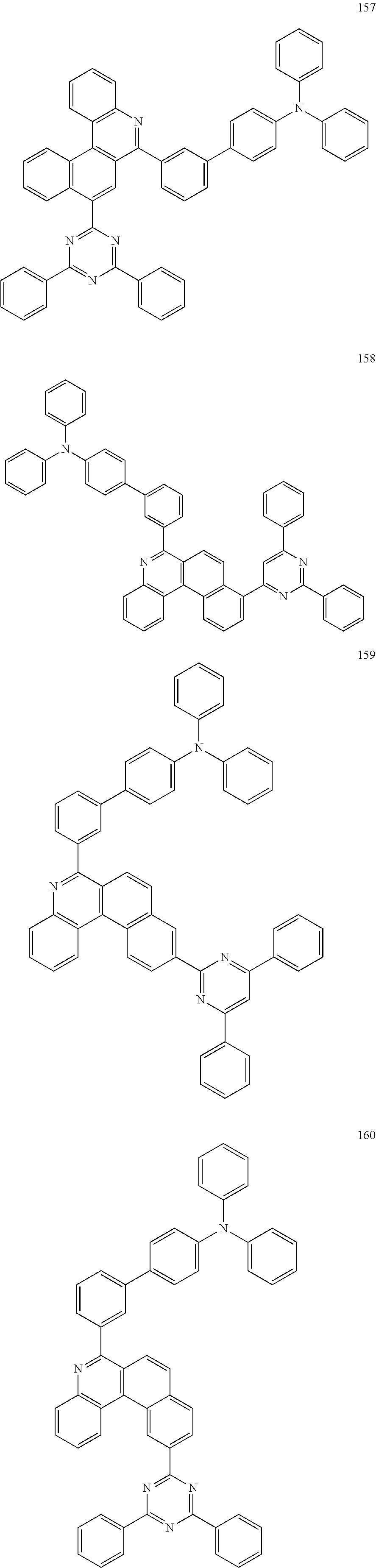 [SMILES: c1ccc(-c2nc(-c3ccccc3)nc(-c3cc4c(-c5cccc(-c6ccc(N(c7ccccc7)c7ccccc7)cc6)c5)nc5ccccc5c4c4ccccc34)n2)cc1 ; c1ccc(-c2cc(-c3cccc4c3ccc3c(-c5cccc(-c6ccc(N(c7ccccc7)c7ccccc7)cc6)c5)nc5ccccc5c34)nc(-c3ccccc3)n2)cc1 ; c1ccc(-c2cc(-c3ccccc3)nc(-c3ccc4c(ccc5c(-c6cccc(-c7ccc(N(c8ccccc8)c8ccccc8)cc7)c6)nc6ccccc6c54)c3)n2)cc1 ; c1ccc(-c2nc(-c3ccccc3)nc(-c3ccc4ccc5c(-c6cccc(-c7ccc(N(c8ccccc8)c8ccccc8)cc7)c6)nc6ccccc6c5c4c3)n2)cc1]

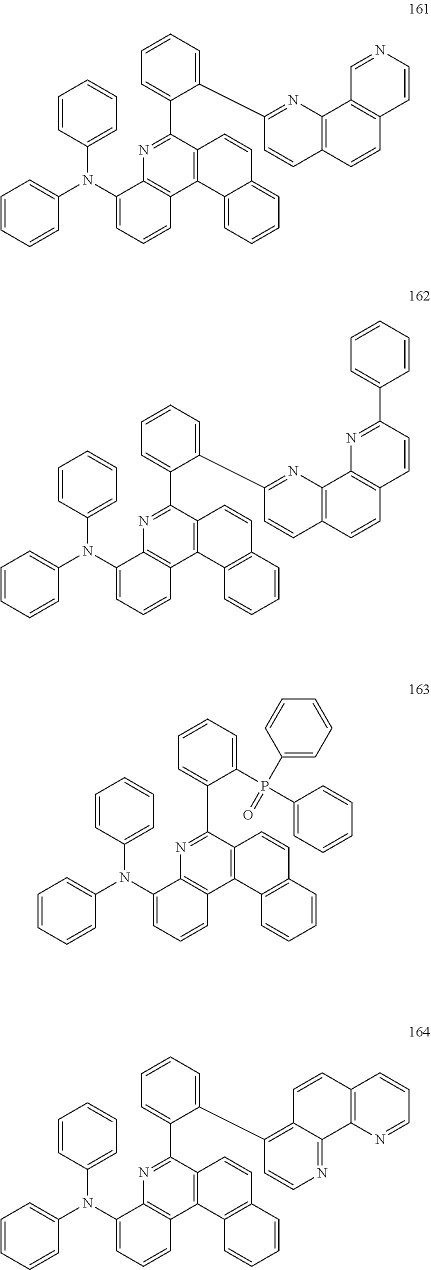 [SMILES: c1ccc(N(c2ccccc2)c2cccc3c2nc(-c2ccccc2-c2ccc4ccc5ccncc5c4n2)c2ccc4ccccc4c23)cc1 ; c1ccc(-c2ccc3ccc4ccc(-c5ccccc5-c5nc6c(N(c7ccccc7)c7ccccc7)cccc6c6c5ccc5ccccc56)nc4c3n2)cc1 ; O=P(c1ccccc1)(c1ccccc1)c1ccccc1-c1nc2c(N(c3ccccc3)c3ccccc3)cccc2c2c1ccc1ccccc12 ; c1ccc(N(c2ccccc2)c2cccc3c2nc(-c2ccccc2-c2ccnc4c2ccc2cccnc24)c2ccc4ccccc4c23)cc1]

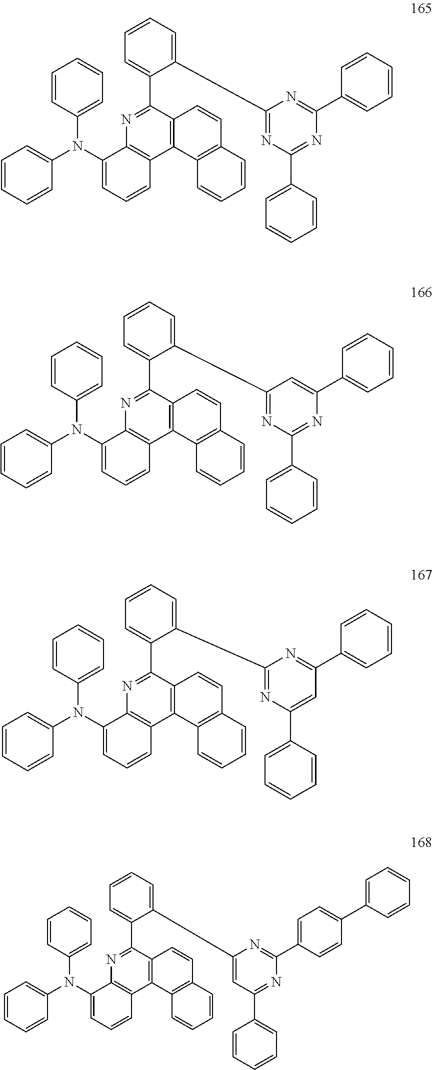 [SMILES: c1ccc(-c2nc(-c3ccccc3)nc(-c3ccccc3-c3nc4c(N(c5ccccc5)c5ccccc5)cccc4c4c3ccc3ccccc34)n2)cc1 ; c1ccc(-c2cc(-c3ccccc3-c3nc4c(N(c5ccccc5)c5ccccc5)cccc4c4c3ccc3ccccc34)nc(-c3ccccc3)n2)cc1 ; c1ccc(-c2cc(-c3ccccc3)nc(-c3ccccc3-c3nc4c(N(c5ccccc5)c5ccccc5)cccc4c4c3ccc3ccccc34)n2)cc1 ; c1ccc(-c2ccc(-c3nc(-c4ccccc4)cc(-c4ccccc4-c4nc5c(N(c6ccccc6)c6ccccc6)cccc5c5c4ccc4ccccc45)n3)cc2)cc1]

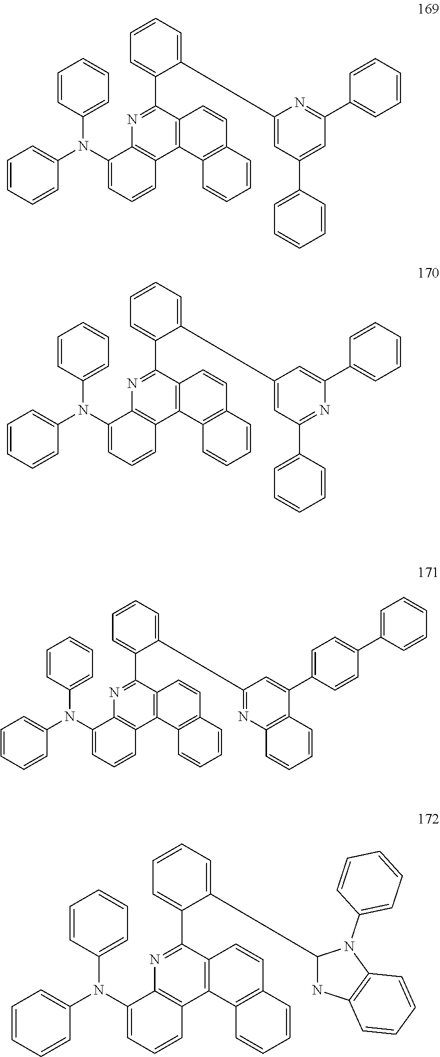 [SMILES: c1ccc(-c2cc(-c3ccccc3)nc(-c3ccccc3-c3nc4c(N(c5ccccc5)c5ccccc5)cccc4c4c3ccc3ccccc34)c2)cc1 ; c1ccc(-c2cc(-c3ccccc3-c3nc4c(N(c5ccccc5)c5ccccc5)cccc4c4c3ccc3ccccc34)cc(-c3ccccc3)n2)cc1 ; c1ccc(-c2ccc(-c3cc(-c4ccccc4-c4nc5c(N(c6ccccc6)c6ccccc6)cccc5c5c4ccc4ccccc45)nc4ccccc34)cc2)cc1 ; c1ccc(N(c2ccccc2)c2cccc3c2nc(-c2ccccc2C2Nc4ccccc4N2c2ccccc2)c2ccc4ccccc4c23)cc1]

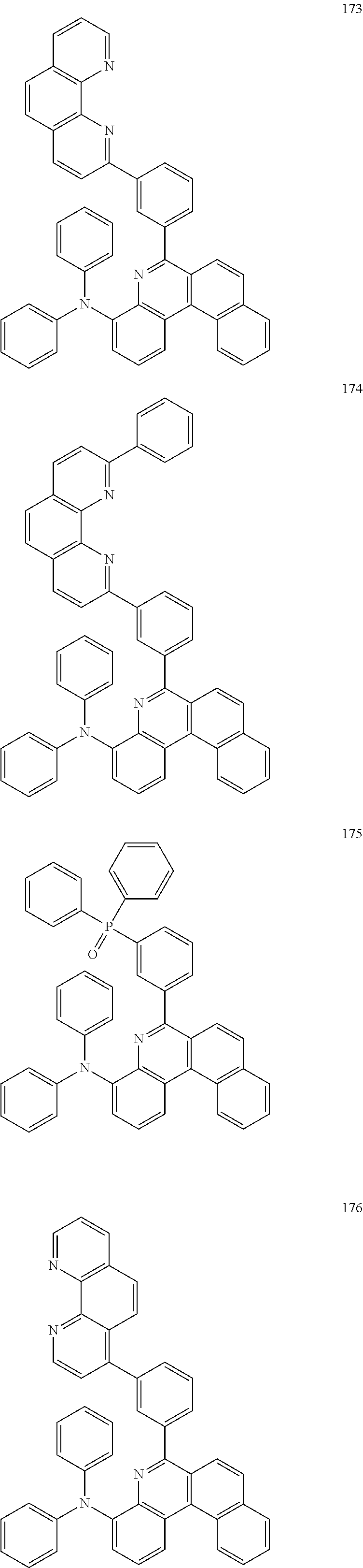

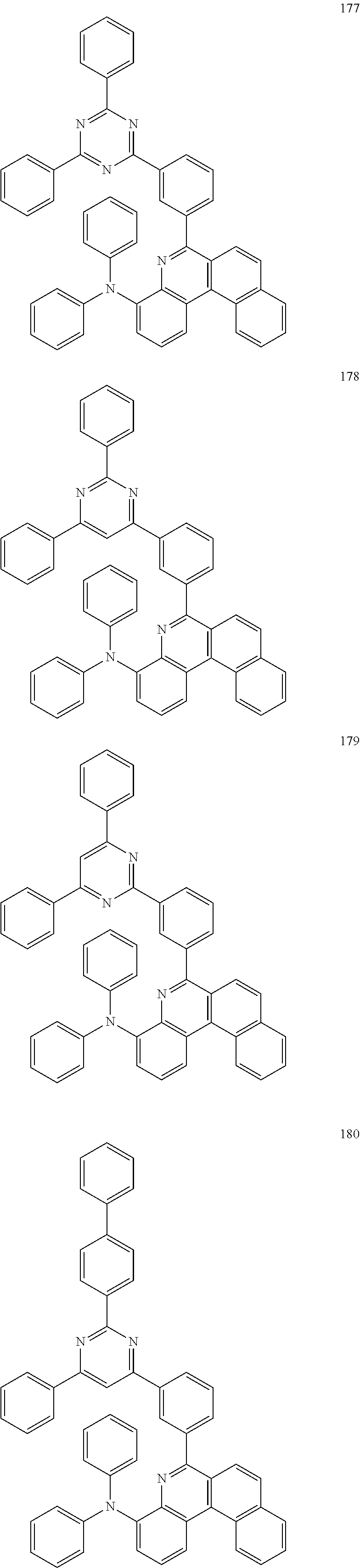 [SMILES: c1ccc(-c2nc(-c3ccccc3)nc(-c3cccc(-c4nc5c(N(c6ccccc6)c6ccccc6)cccc5c5c4ccc4ccccc45)c3)n2)cc1 ; c1ccc(-c2cc(-c3cccc(-c4nc5c(N(c6ccccc6)c6ccccc6)cccc5c5c4ccc4ccccc45)c3)nc(-c3ccccc3)n2)cc1 ; c1ccc(-c2cc(-c3ccccc3)nc(-c3cccc(-c4nc5c(N(c6ccccc6)c6ccccc6)cccc5c5c4ccc4ccccc45)c3)n2)cc1 ; c1ccc(-c2ccc(-c3nc(-c4ccccc4)cc(-c4cccc(-c5nc6c(N(c7ccccc7)c7ccccc7)cccc6c6c5ccc5ccccc56)c4)n3)cc2)cc1]

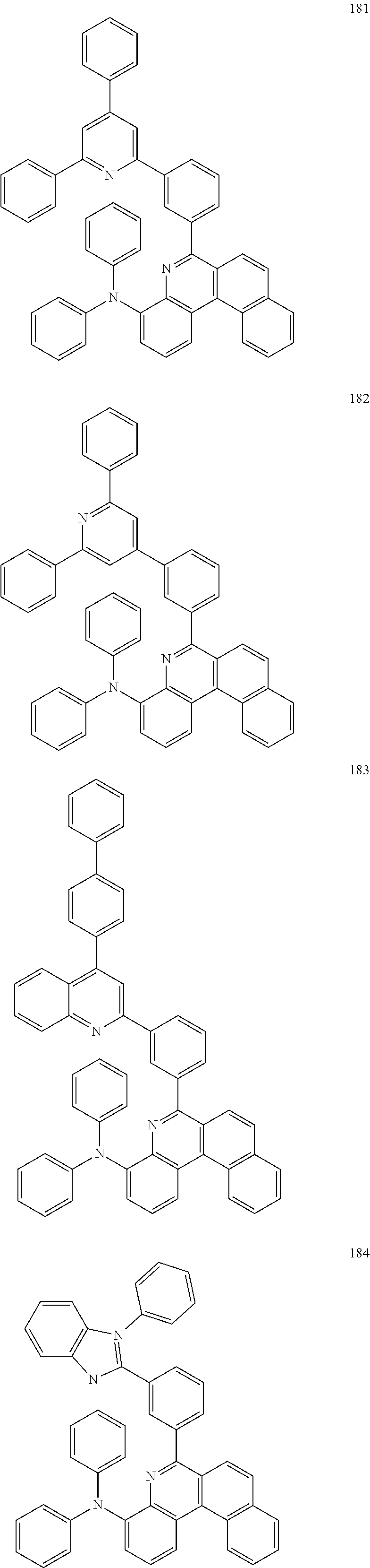

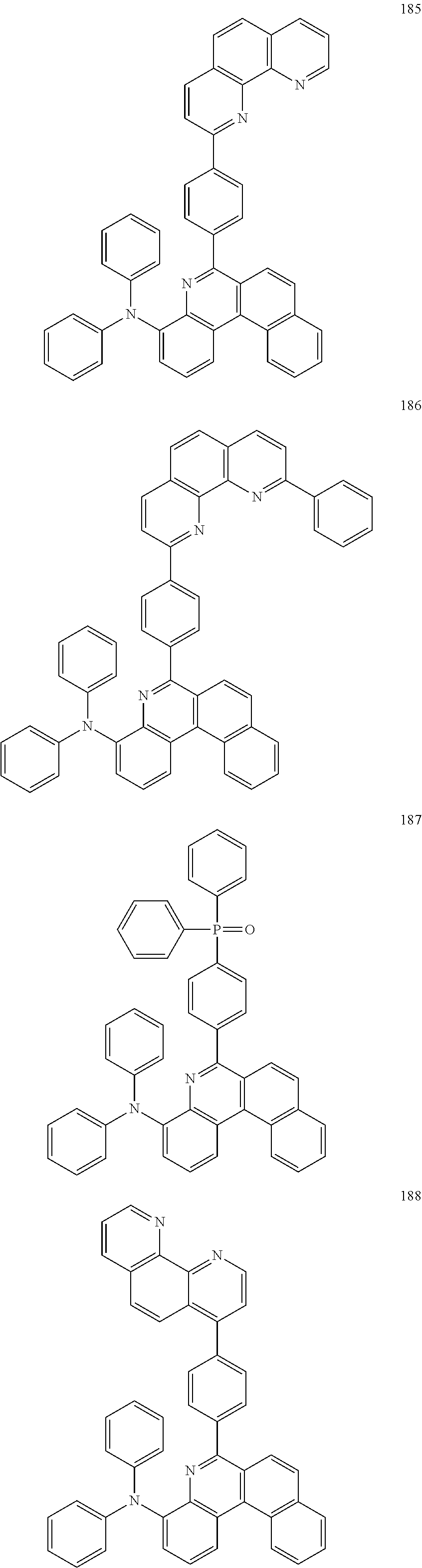 [SMILES: c1ccc(N(c2ccccc2)c2cccc3c2nc(-c2ccc(-c4ccc5ccc6cccnc6c5n4)cc2)c2ccc4ccccc4c23)cc1 ; c1ccc(-c2ccc3ccc4ccc(-c5ccc(-c6nc7c(N(c8ccccc8)c8ccccc8)cccc7c7c6ccc6ccccc67)cc5)nc4c3n2)cc1 ; O=P(c1ccccc1)(c1ccccc1)c1ccc(-c2nc3c(N(c4ccccc4)c4ccccc4)cccc3c3c2ccc2ccccc23)cc1 ; c1ccc(N(c2ccccc2)c2cccc3c2nc(-c2ccc(-c4ccnc5c4ccc4cccnc45)cc2)c2ccc4ccccc4c23)cc1]

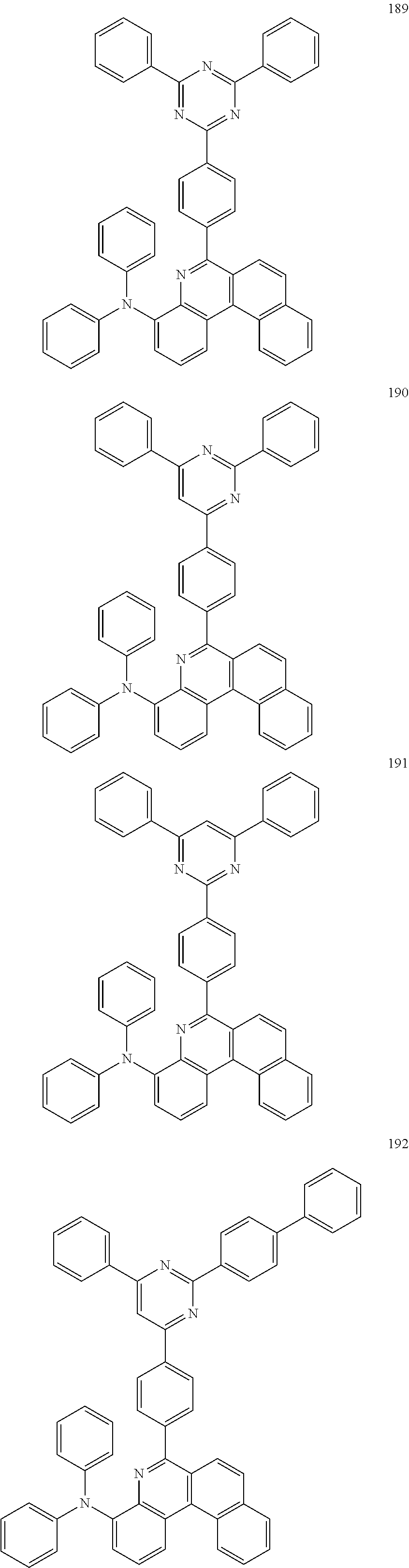

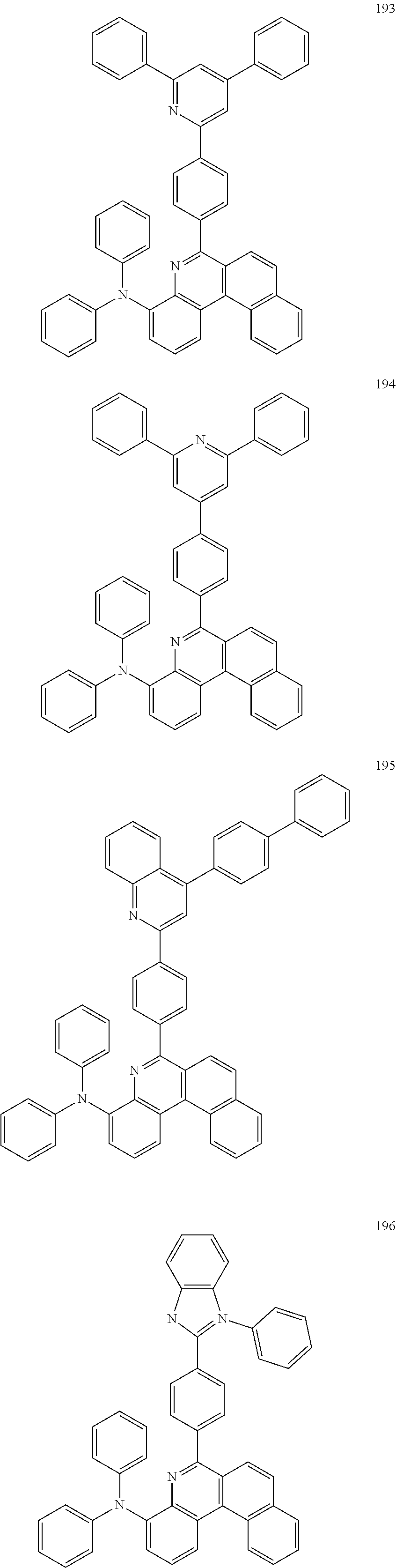

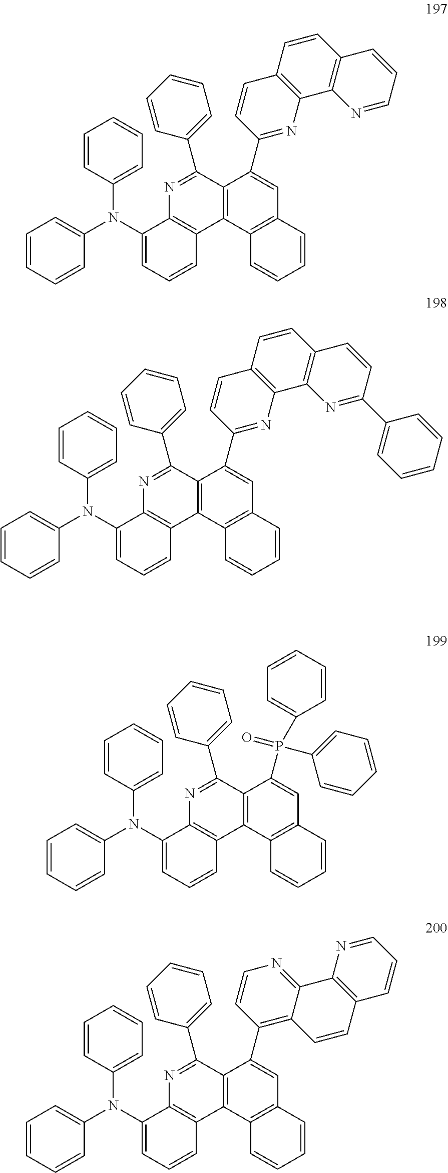 [SMILES: c1ccc(-c2nc3c(N(c4ccccc4)c4ccccc4)cccc3c3c2c(-c2ccc4ccc5cccnc5c4n2)cc2ccccc23)cc1 ; c1ccc(-c2ccc3ccc4ccc(-c5cc6ccccc6c6c5c(-c5ccccc5)nc5c(N(c7ccccc7)c7ccccc7)cccc56)nc4c3n2)cc1 ; O=P(c1ccccc1)(c1ccccc1)c1cc2ccccc2c2c1c(-c1ccccc1)nc1c(N(c3ccccc3)c3ccccc3)cccc12 ; c1ccc(-c2nc3c(N(c4ccccc4)c4ccccc4)cccc3c3c2c(-c2ccnc4c2ccc2cccnc24)cc2ccccc23)cc1]

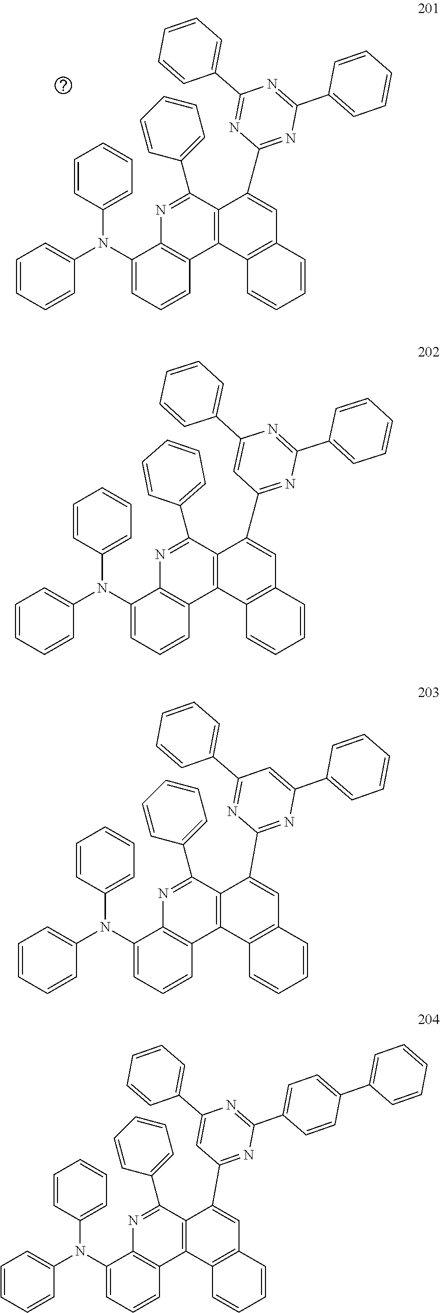 [SMILES: c1ccc(-c2nc(-c3ccccc3)nc(-c3cc4ccccc4c4c3c(-c3ccccc3)nc3c(N(c5ccccc5)c5ccccc5)cccc34)n2)cc1 ; c1ccc(-c2cc(-c3cc4ccccc4c4c3c(-c3ccccc3)nc3c(N(c5ccccc5)c5ccccc5)cccc34)nc(-c3ccccc3)n2)cc1 ; c1ccc(-c2cc(-c3ccccc3)nc(-c3cc4ccccc4c4c3c(-c3ccccc3)nc3c(N(c5ccccc5)c5ccccc5)cccc34)n2)cc1 ; c1ccc(-c2ccc(-c3nc(-c4ccccc4)cc(-c4cc5ccccc5c5c4c(-c4ccccc4)nc4c(N(c6ccccc6)c6ccccc6)cccc45)n3)cc2)cc1]

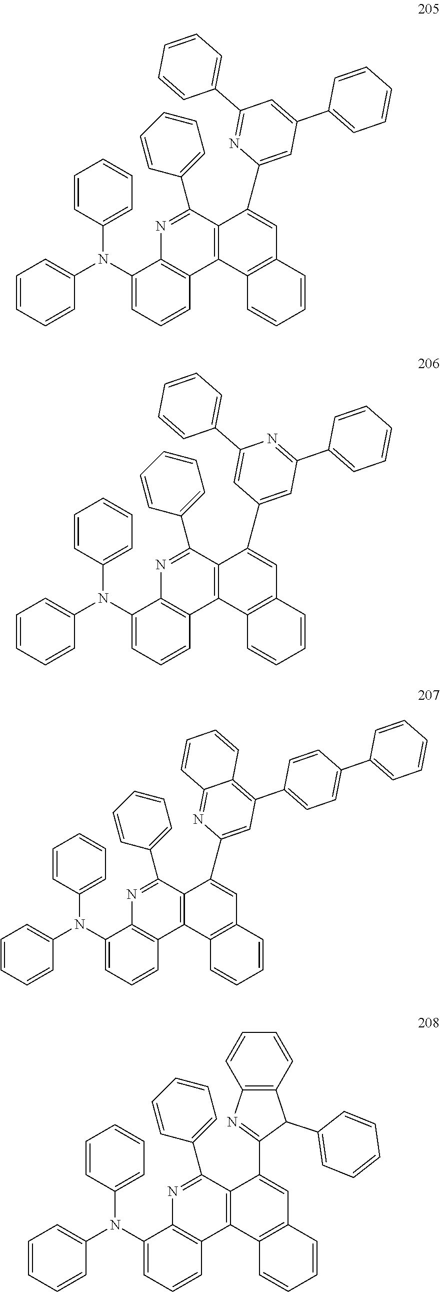 [SMILES: c1ccc(-c2cc(-c3ccccc3)nc(-c3cc4ccccc4c4c3c(-c3ccccc3)nc3c(N(c5ccccc5)c5ccccc5)cccc34)c2)cc1 ; c1ccc(-c2cc(-c3cc4ccccc4c4c3c(-c3ccccc3)nc3c(N(c5ccccc5)c5ccccc5)cccc34)cc(-c3ccccc3)n2)cc1 ; c1ccc(-c2ccc(-c3cc(-c4cc5ccccc5c5c4c(-c4ccccc4)nc4c(N(c6ccccc6)c6ccccc6)cccc45)nc4ccccc34)cc2)cc1 ; c1ccc(-c2nc3c(N(c4ccccc4)c4ccccc4)cccc3c3c2c(C2=Nc4ccccc4C2c2ccccc2)cc2ccccc23)cc1]

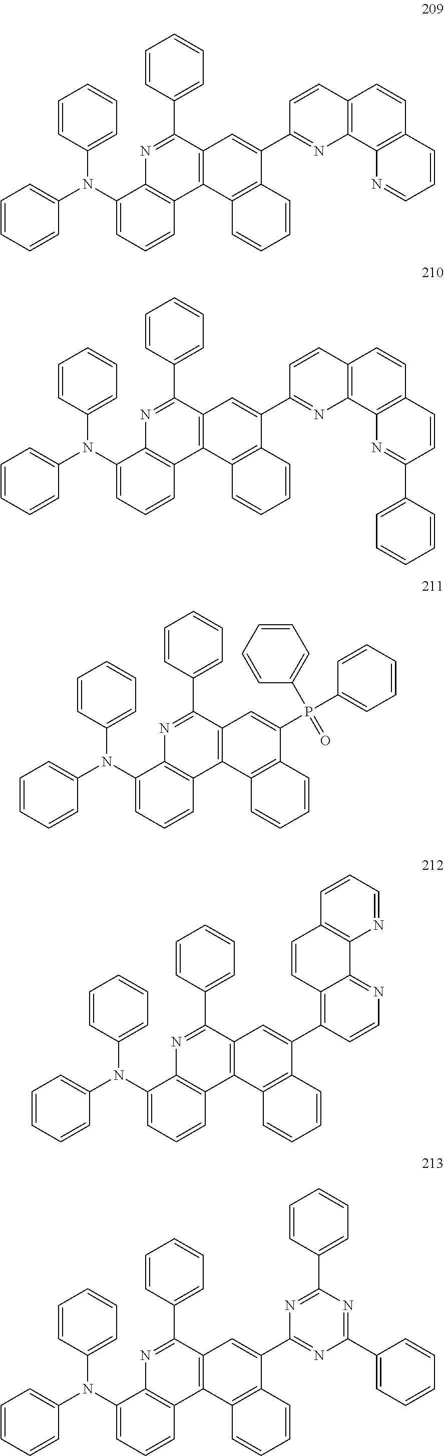 [SMILES: c1ccc(-c2nc3c(N(c4ccccc4)c4ccccc4)cccc3c3c2cc(-c2ccc4ccc5cccnc5c4n2)c2ccccc23)cc1 ; c1ccc(-c2ccc3ccc4ccc(-c5cc6c(-c7ccccc7)nc7c(N(c8ccccc8)c8ccccc8)cccc7c6c6ccccc56)nc4c3n2)cc1 ; O=P(c1ccccc1)(c1ccccc1)c1cc2c(-c3ccccc3)nc3c(N(c4ccccc4)c4ccccc4)cccc3c2c2ccccc12 ; c1ccc(-c2nc3c(N(c4ccccc4)c4ccccc4)cccc3c3c2cc(-c2ccnc4c2ccc2cccnc24)c2ccccc23)cc1 ; c1ccc(-c2nc(-c3ccccc3)nc(-c3cc4c(-c5ccccc5)nc5c(N(c6ccccc6)c6ccccc6)cccc5c4c4ccccc34)n2)cc1]

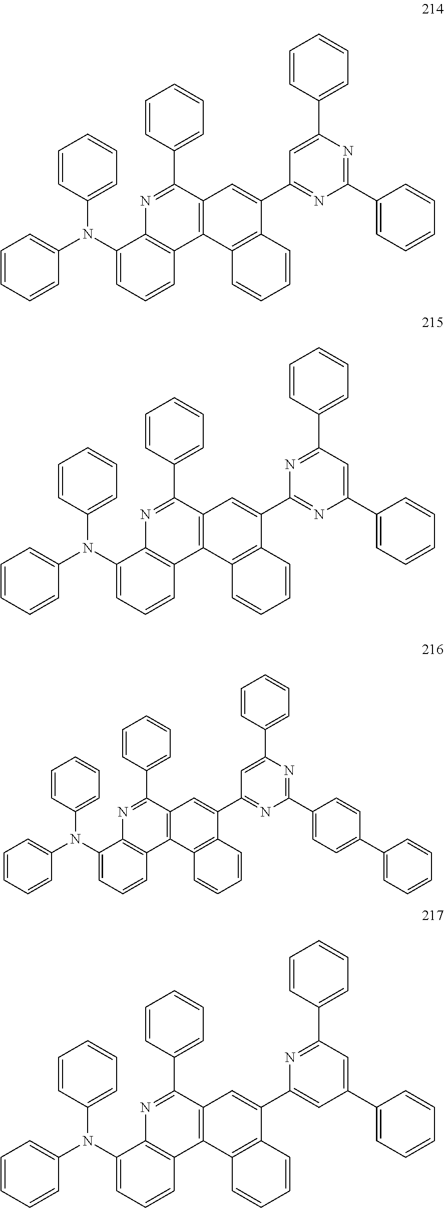 [SMILES: c1ccc(-c2cc(-c3cc4c(-c5ccccc5)nc5c(N(c6ccccc6)c6ccccc6)cccc5c4c4ccccc34)nc(-c3ccccc3)n2)cc1 ; c1ccc(-c2cc(-c3ccccc3)nc(-c3cc4c(-c5ccccc5)nc5c(N(c6ccccc6)c6ccccc6)cccc5c4c4ccccc34)n2)cc1 ; c1ccc(-c2ccc(-c3nc(-c4ccccc4)cc(-c4cc5c(-c6ccccc6)nc6c(N(c7ccccc7)c7ccccc7)cccc6c5c5ccccc45)n3)cc2)cc1 ; c1ccc(-c2cc(-c3ccccc3)nc(-c3cc4c(-c5ccccc5)nc5c(N(c6ccccc6)c6ccccc6)cccc5c4c4ccccc34)c2)cc1]

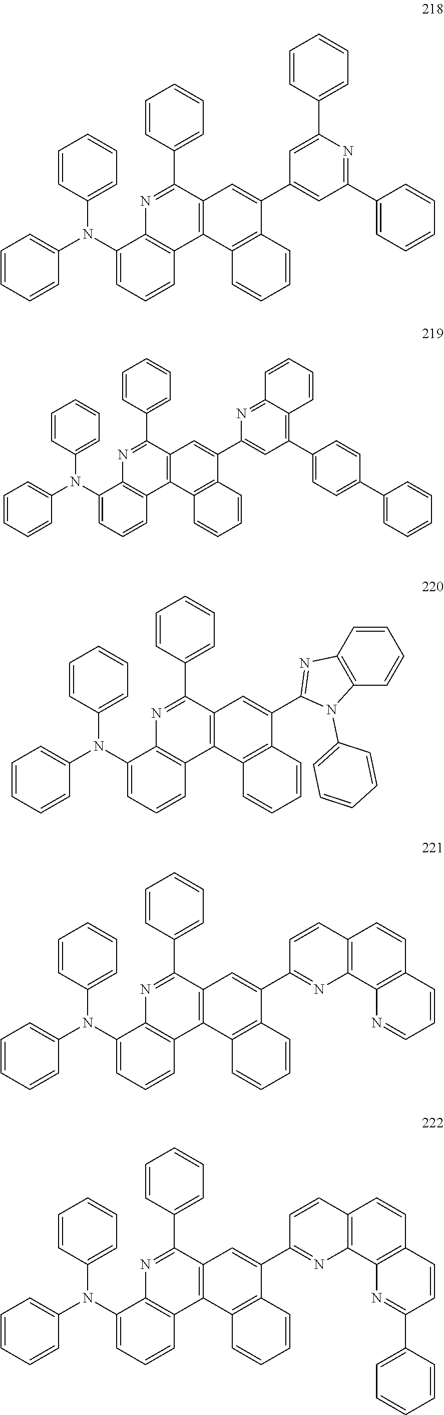 [SMILES: c1ccc(-c2cc(-c3cc4c(-c5ccccc5)nc5c(N(c6ccccc6)c6ccccc6)cccc5c4c4ccccc34)cc(-c3ccccc3)n2)cc1 ; c1ccc(-c2ccc(-c3cc(-c4cc5c(-c6ccccc6)nc6c(N(c7ccccc7)c7ccccc7)cccc6c5c5ccccc45)nc4ccccc34)cc2)cc1 ; c1ccc(-c2nc3c(N(c4ccccc4)c4ccccc4)cccc3c3c2cc(-c2nc4ccccc4n2-c2ccccc2)c2ccccc23)cc1 ; c1ccc(-c2nc3c(N(c4ccccc4)c4ccccc4)cccc3c3c2cc(-c2ccc4ccc5cccnc5c4n2)c2ccccc23)cc1 ; c1ccc(-c2ccc3ccc4ccc(-c5cc6c(-c7ccccc7)nc7c(N(c8ccccc8)c8ccccc8)cccc7c6c6ccccc56)nc4c3n2)cc1]

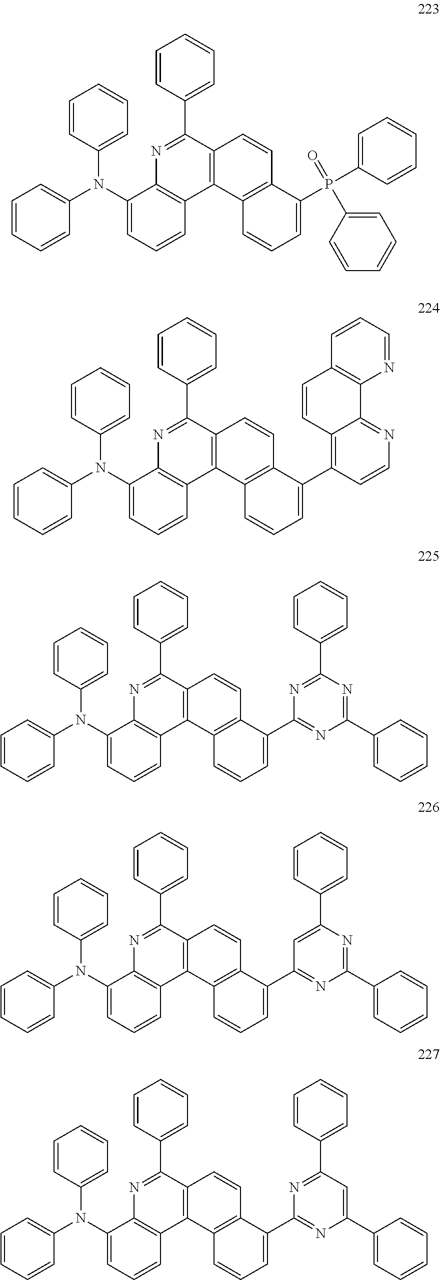 [SMILES: O=P(c1ccccc1)(c1ccccc1)c1cccc2c1ccc1c(-c3ccccc3)nc3c(N(c4ccccc4)c4ccccc4)cccc3c12 ; c1ccc(-c2nc3c(N(c4ccccc4)c4ccccc4)cccc3c3c2ccc2c(-c4ccnc5c4ccc4cccnc45)cccc23)cc1 ; c1ccc(-c2nc(-c3ccccc3)nc(-c3cccc4c3ccc3c(-c5ccccc5)nc5c(N(c6ccccc6)c6ccccc6)cccc5c34)n2)cc1 ; c1ccc(-c2cc(-c3cccc4c3ccc3c(-c5ccccc5)nc5c(N(c6ccccc6)c6ccccc6)cccc5c34)nc(-c3ccccc3)n2)cc1 ; c1ccc(-c2cc(-c3ccccc3)nc(-c3cccc4c3ccc3c(-c5ccccc5)nc5c(N(c6ccccc6)c6ccccc6)cccc5c34)n2)cc1]

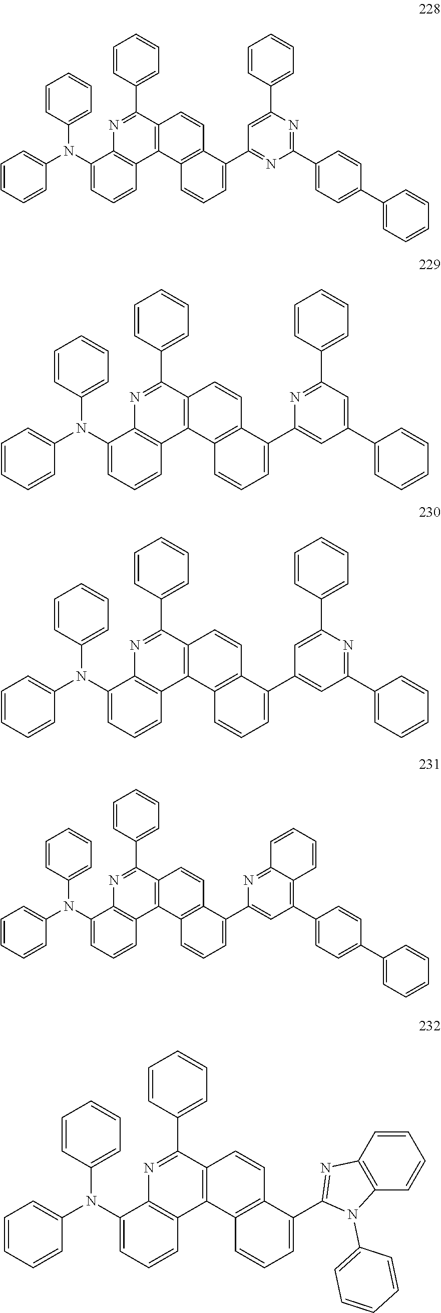 [SMILES: c1ccc(-c2ccc(-c3nc(-c4ccccc4)cc(-c4cccc5c4ccc4c(-c6ccccc6)nc6c(N(c7ccccc7)c7ccccc7)cccc6c45)n3)cc2)cc1 ; c1ccc(-c2cc(-c3ccccc3)nc(-c3cccc4c3ccc3c(-c5ccccc5)nc5c(N(c6ccccc6)c6ccccc6)cccc5c34)c2)cc1 ; c1ccc(-c2cc(-c3cccc4c3ccc3c(-c5ccccc5)nc5c(N(c6ccccc6)c6ccccc6)cccc5c34)cc(-c3ccccc3)n2)cc1 ; c1ccc(-c2ccc(-c3cc(-c4cccc5c4ccc4c(-c6ccccc6)nc6c(N(c7ccccc7)c7ccccc7)cccc6c45)nc4ccccc34)cc2)cc1 ; c1ccc(-c2nc3c(N(c4ccccc4)c4ccccc4)cccc3c3c2ccc2c(-c4nc5ccccc5n4-c4ccccc4)cccc23)cc1]

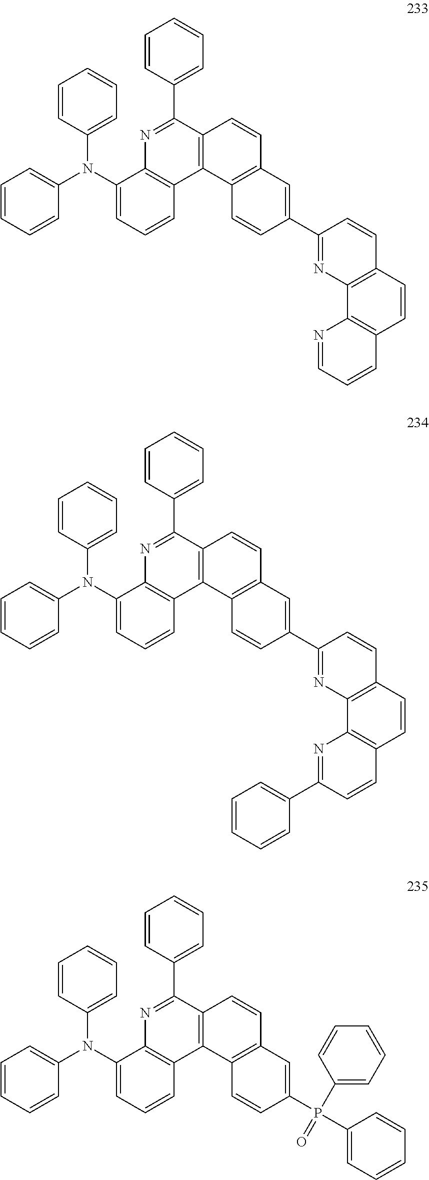 [SMILES: c1ccc(-c2nc3c(N(c4ccccc4)c4ccccc4)cccc3c3c2ccc2cc(-c4ccc5ccc6cccnc6c5n4)ccc23)cc1 ; c1ccc(-c2ccc3ccc4ccc(-c5ccc6c(ccc7c(-c8ccccc8)nc8c(N(c9ccccc9)c9ccccc9)cccc8c76)c5)nc4c3n2)cc1 ; O=P(c1ccccc1)(c1ccccc1)c1ccc2c(ccc3c(-c4ccccc4)nc4c(N(c5ccccc5)c5ccccc5)cccc4c32)c1]

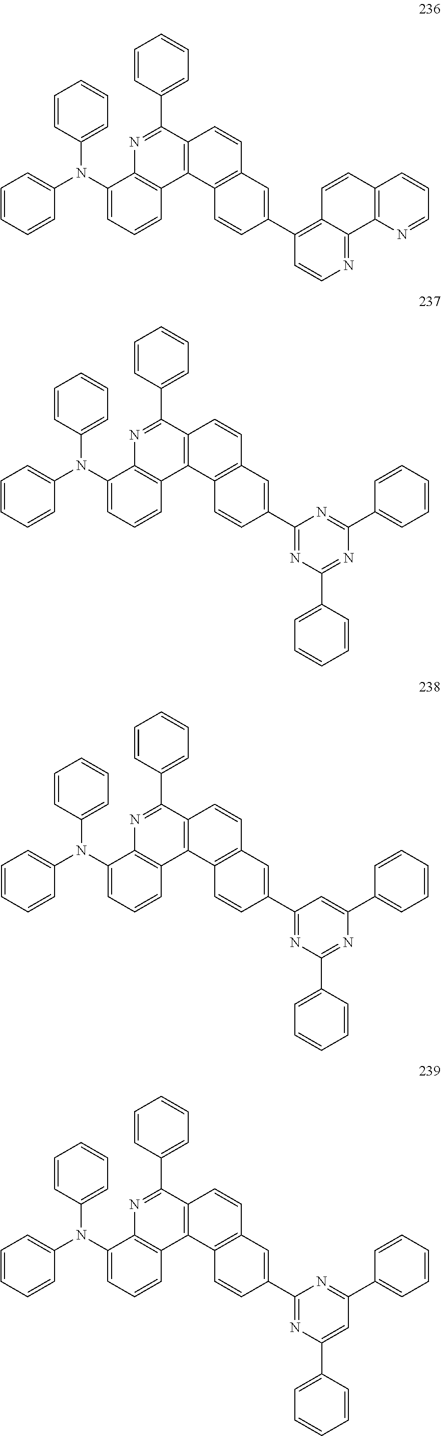 [SMILES: c1ccc(-c2nc3c(N(c4ccccc4)c4ccccc4)cccc3c3c2ccc2cc(-c4ccnc5c4ccc4cccnc45)ccc23)cc1 ; c1ccc(-c2nc(-c3ccccc3)nc(-c3ccc4c(ccc5c(-c6ccccc6)nc6c(N(c7ccccc7)c7ccccc7)cccc6c54)c3)n2)cc1 ; c1ccc(-c2cc(-c3ccc4c(ccc5c(-c6ccccc6)nc6c(N(c7ccccc7)c7ccccc7)cccc6c54)c3)nc(-c3ccccc3)n2)cc1 ; c1ccc(-c2cc(-c3ccccc3)nc(-c3ccc4c(ccc5c(-c6ccccc6)nc6c(N(c7ccccc7)c7ccccc7)cccc6c54)c3)n2)cc1]

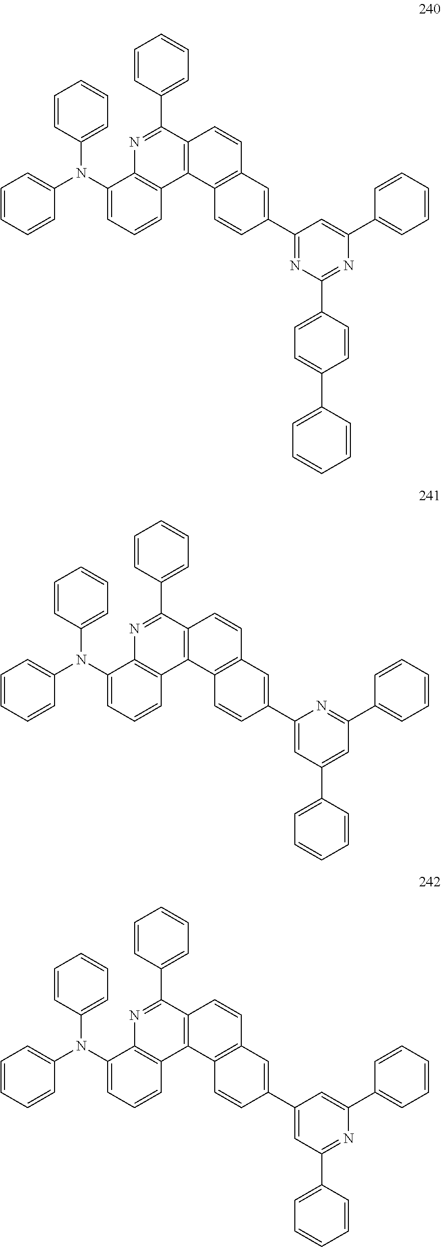 [SMILES: c1ccc(-c2ccc(-c3nc(-c4ccccc4)cc(-c4ccc5c(ccc6c(-c7ccccc7)nc7c(N(c8ccccc8)c8ccccc8)cccc7c65)c4)n3)cc2)cc1 ; c1ccc(-c2cc(-c3ccccc3)nc(-c3ccc4c(ccc5c(-c6ccccc6)nc6c(N(c7ccccc7)c7ccccc7)cccc6c54)c3)c2)cc1 ; c1ccc(-c2cc(-c3ccc4c(ccc5c(-c6ccccc6)nc6c(N(c7ccccc7)c7ccccc7)cccc6c54)c3)cc(-c3ccccc3)n2)cc1]

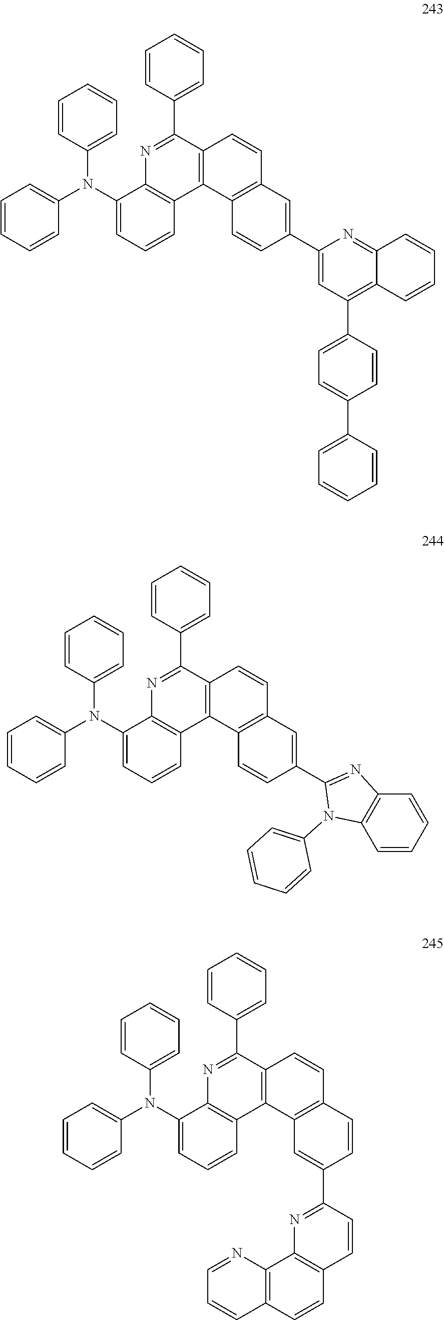 [SMILES: c1ccc(-c2ccc(-c3cc(-c4ccc5c(ccc6c(-c7ccccc7)nc7c(N(c8ccccc8)c8ccccc8)cccc7c65)c4)nc4ccccc34)cc2)cc1 ; c1ccc(-c2nc3c(N(c4ccccc4)c4ccccc4)cccc3c3c2ccc2cc(-c4nc5ccccc5n4-c4ccccc4)ccc23)cc1 ; c1ccc(-c2nc3c(N(c4ccccc4)c4ccccc4)cccc3c3c2ccc2ccc(-c4ccc5ccc6cccnc6c5n4)cc23)cc1]

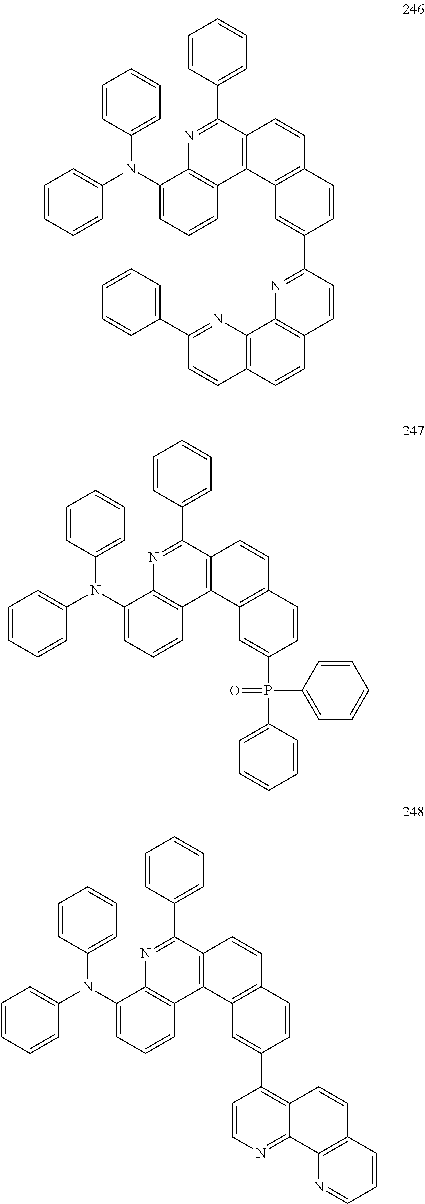 [SMILES: c1ccc(-c2ccc3ccc4ccc(-c5ccc6ccc7c(-c8ccccc8)nc8c(N(c9ccccc9)c9ccccc9)cccc8c7c6c5)nc4c3n2)cc1 ; O=P(c1ccccc1)(c1ccccc1)c1ccc2ccc3c(-c4ccccc4)nc4c(N(c5ccccc5)c5ccccc5)cccc4c3c2c1 ; c1ccc(-c2nc3c(N(c4ccccc4)c4ccccc4)cccc3c3c2ccc2ccc(-c4ccnc5c4ccc4cccnc45)cc23)cc1]

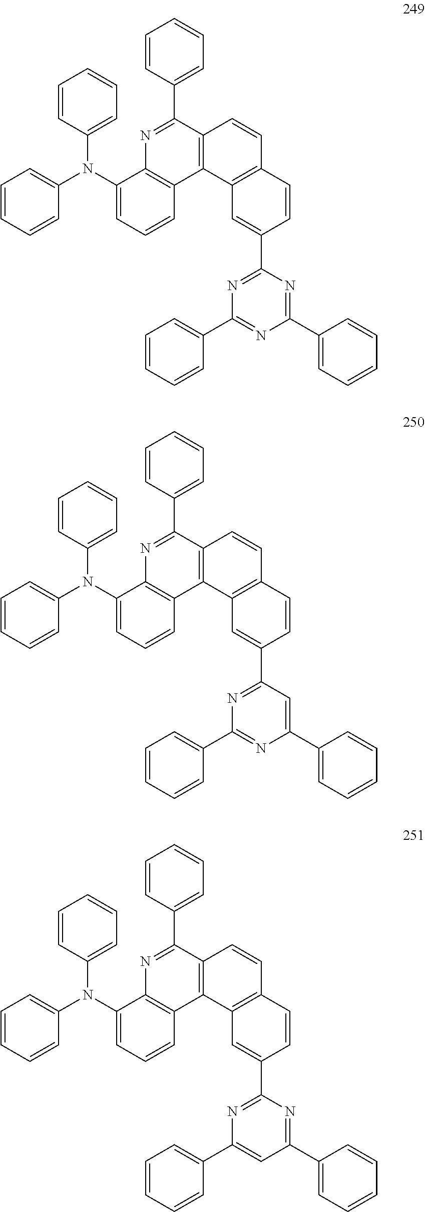

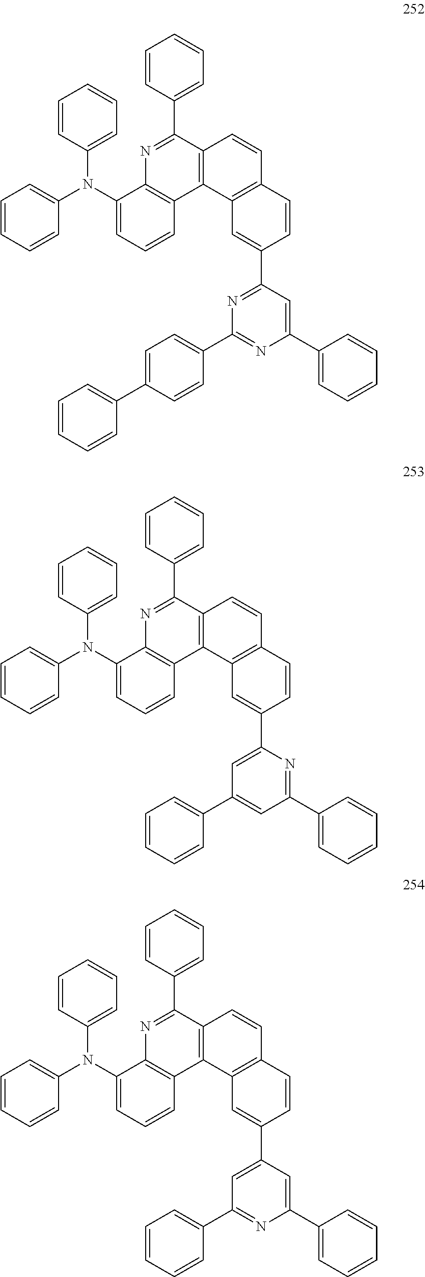

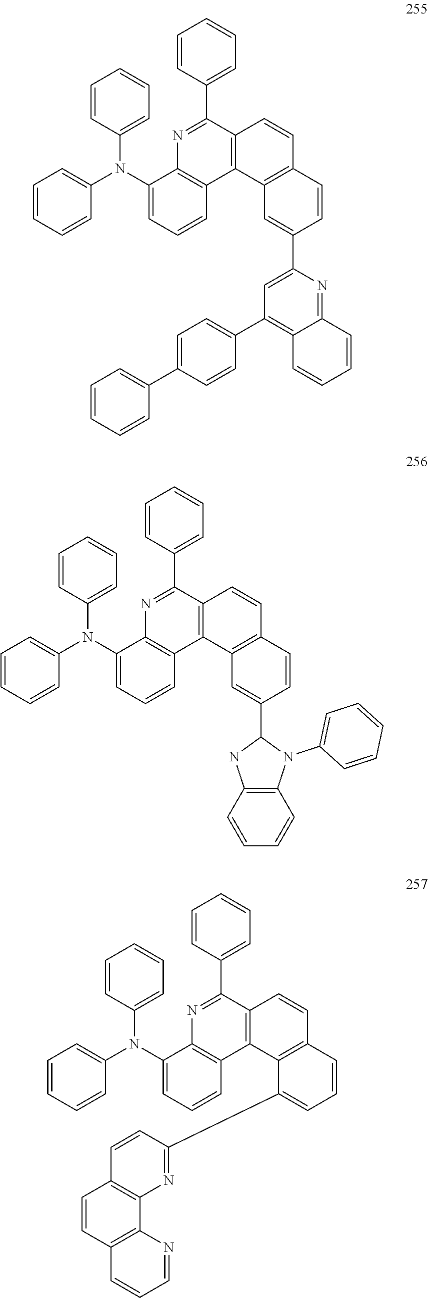 [SMILES: c1ccc(-c2ccc(-c3cc(-c4ccc5ccc6c(-c7ccccc7)nc7c(N(c8ccccc8)c8ccccc8)cccc7c6c5c4)nc4ccccc34)cc2)cc1 ; c1ccc(-c2nc3c(N(c4ccccc4)c4ccccc4)cccc3c3c2ccc2ccc(C4Nc5ccccc5N4c4ccccc4)cc23)cc1 ; c1ccc(-c2nc3c(N(c4ccccc4)c4ccccc4)cccc3c3c2ccc2cccc(-c4ccc5ccc6cccnc6c5n4)c23)cc1]

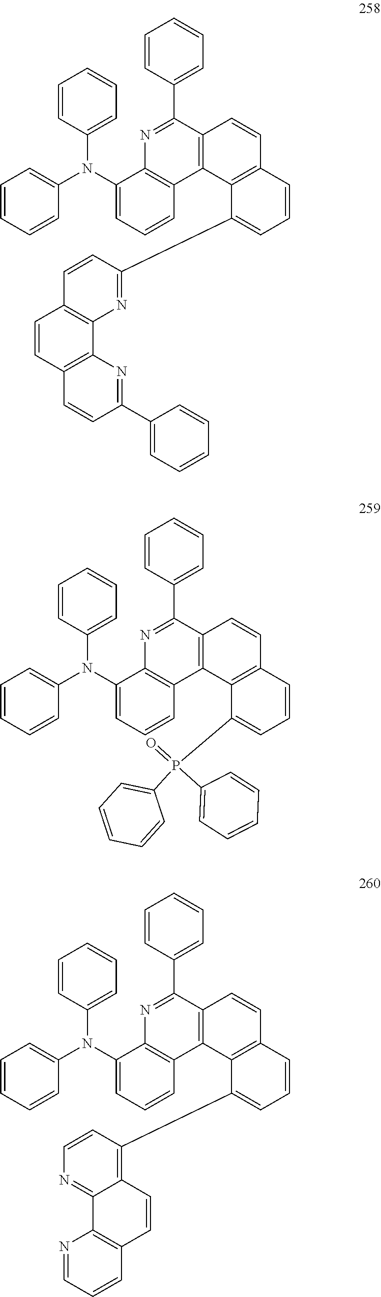 [SMILES: c1ccc(-c2ccc3ccc4ccc(-c5cccc6ccc7c(-c8ccccc8)nc8c(N(c9ccccc9)c9ccccc9)cccc8c7c56)nc4c3n2)cc1 ; O=P(c1ccccc1)(c1ccccc1)c1cccc2ccc3c(-c4ccccc4)nc4c(N(c5ccccc5)c5ccccc5)cccc4c3c12 ; c1ccc(-c2nc3c(N(c4ccccc4)c4ccccc4)cccc3c3c2ccc2cccc(-c4ccnc5c4ccc4cccnc45)c23)cc1]

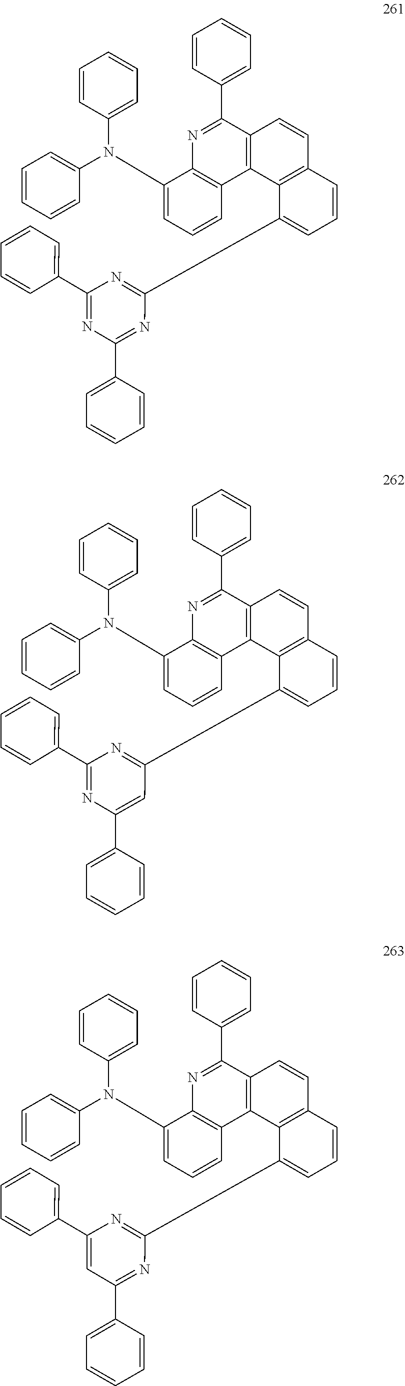 [SMILES: c1ccc(-c2nc(-c3ccccc3)nc(-c3cccc4ccc5c(-c6ccccc6)nc6c(N(c7ccccc7)c7ccccc7)cccc6c5c34)n2)cc1 ; c1ccc(-c2cc(-c3cccc4ccc5c(-c6ccccc6)nc6c(N(c7ccccc7)c7ccccc7)cccc6c5c34)nc(-c3ccccc3)n2)cc1 ; c1ccc(-c2cc(-c3ccccc3)nc(-c3cccc4ccc5c(-c6ccccc6)nc6c(N(c7ccccc7)c7ccccc7)cccc6c5c34)n2)cc1]

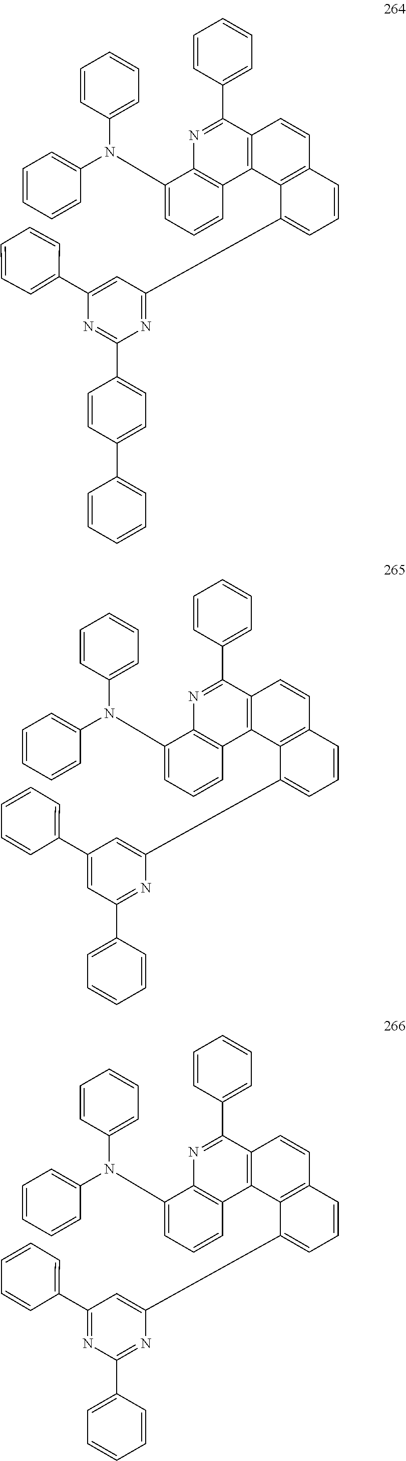 [SMILES: c1ccc(-c2ccc(-c3nc(-c4ccccc4)cc(-c4cccc5ccc6c(-c7ccccc7)nc7c(N(c8ccccc8)c8ccccc8)cccc7c6c45)n3)cc2)cc1 ; c1ccc(-c2cc(-c3ccccc3)nc(-c3cccc4ccc5c(-c6ccccc6)nc6c(N(c7ccccc7)c7ccccc7)cccc6c5c34)c2)cc1 ; c1ccc(-c2cc(-c3cccc4ccc5c(-c6ccccc6)nc6c(N(c7ccccc7)c7ccccc7)cccc6c5c34)nc(-c3ccccc3)n2)cc1]

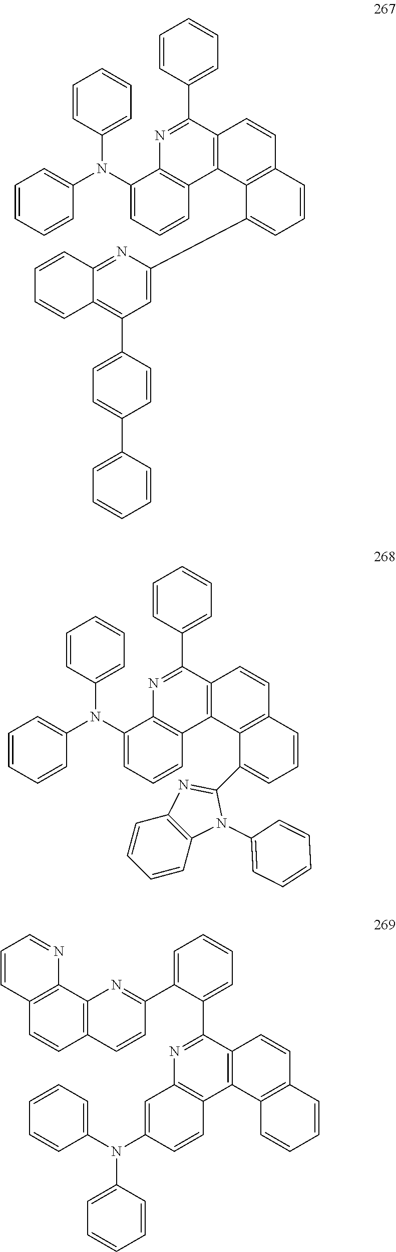 [SMILES: c1ccc(-c2ccc(-c3cc(-c4cccc5ccc6c(-c7ccccc7)nc7c(N(c8ccccc8)c8ccccc8)cccc7c6c45)nc4ccccc34)cc2)cc1 ; c1ccc(-c2nc3c(N(c4ccccc4)c4ccccc4)cccc3c3c2ccc2cccc(-c4nc5ccccc5n4-c4ccccc4)c23)cc1 ; c1ccc(N(c2ccccc2)c2ccc3c(c2)nc(-c2ccccc2-c2ccc4ccc5cccnc5c4n2)c2ccc4ccccc4c23)cc1]

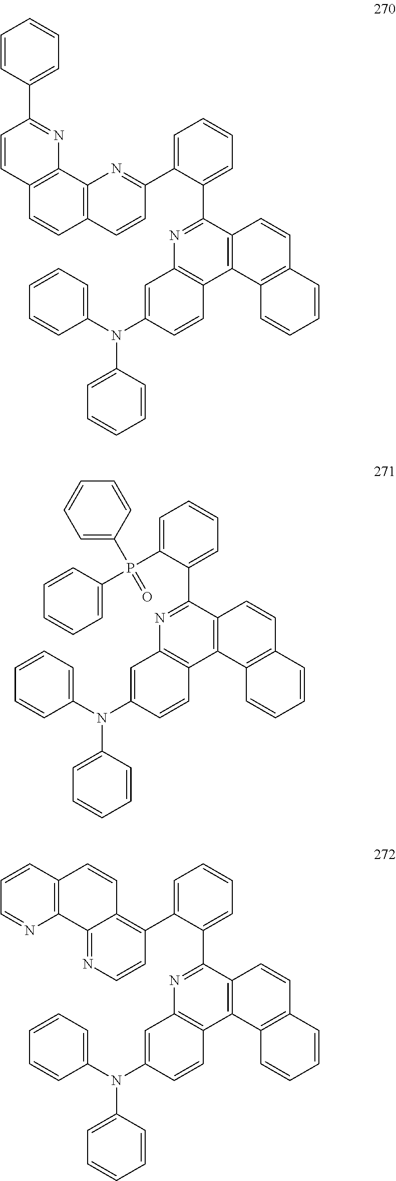

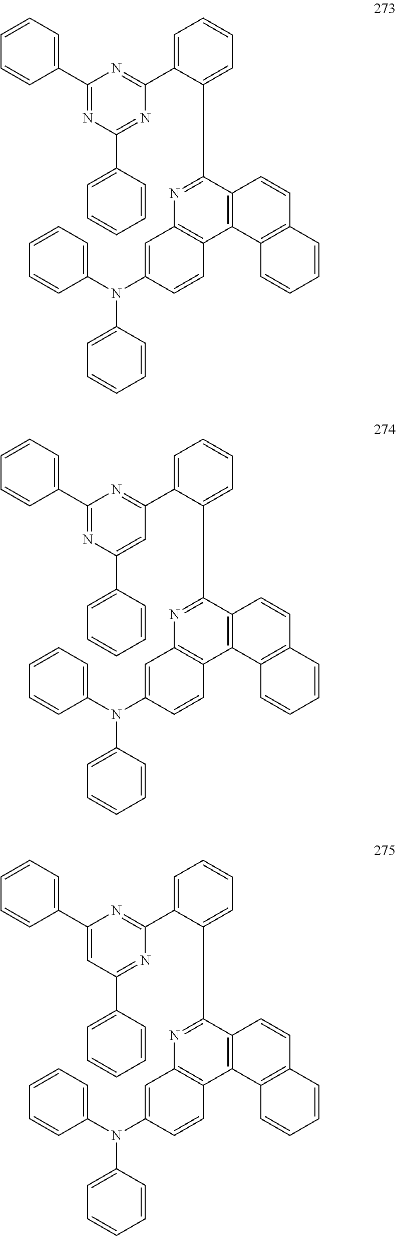 [SMILES: c1ccc(-c2nc(-c3ccccc3)nc(-c3ccccc3-c3nc4cc(N(c5ccccc5)c5ccccc5)ccc4c4c3ccc3ccccc34)n2)cc1 ; c1ccc(-c2cc(-c3ccccc3-c3nc4cc(N(c5ccccc5)c5ccccc5)ccc4c4c3ccc3ccccc34)nc(-c3ccccc3)n2)cc1 ; c1ccc(-c2cc(-c3ccccc3)nc(-c3ccccc3-c3nc4cc(N(c5ccccc5)c5ccccc5)ccc4c4c3ccc3ccccc34)n2)cc1]

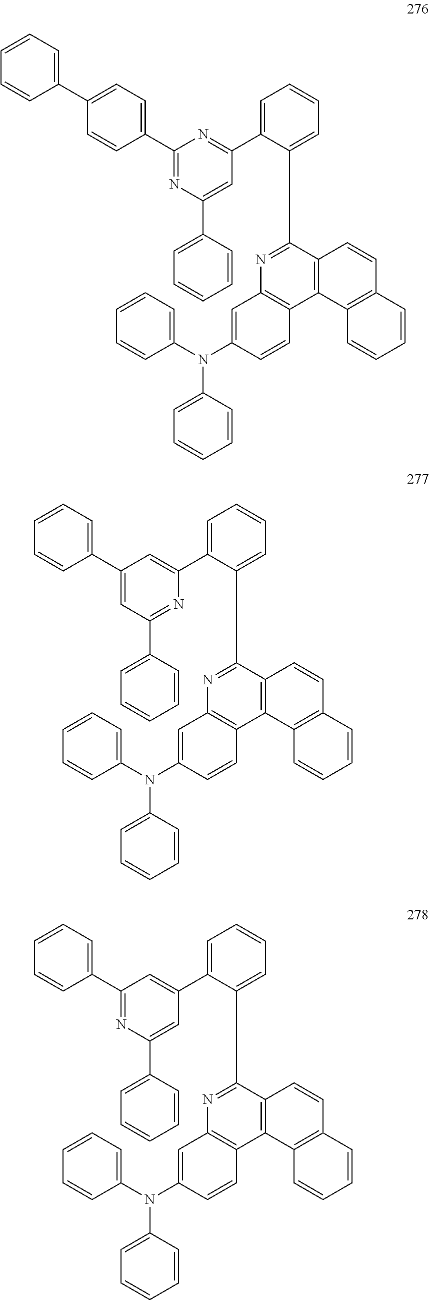 [SMILES: c1ccc(-c2ccc(-c3nc(-c4ccccc4)cc(-c4ccccc4-c4nc5cc(N(c6ccccc6)c6ccccc6)ccc5c5c4ccc4ccccc45)n3)cc2)cc1 ; c1ccc(-c2cc(-c3ccccc3)nc(-c3ccccc3-c3nc4cc(N(c5ccccc5)c5ccccc5)ccc4c4c3ccc3ccccc34)c2)cc1 ; c1ccc(-c2cc(-c3ccccc3-c3nc4cc(N(c5ccccc5)c5ccccc5)ccc4c4c3ccc3ccccc34)cc(-c3ccccc3)n2)cc1]

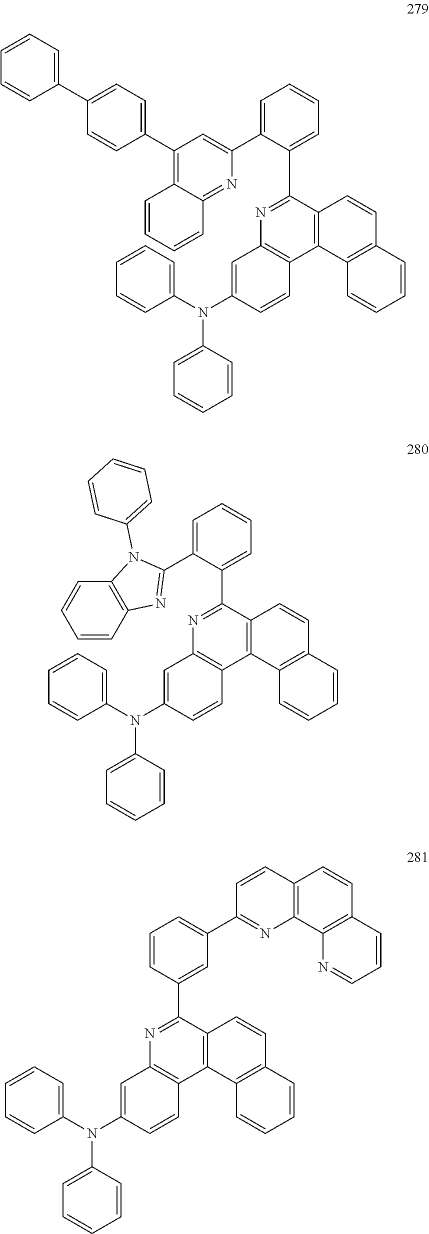 [SMILES: c1ccc(-c2ccc(-c3cc(-c4ccccc4-c4nc5cc(N(c6ccccc6)c6ccccc6)ccc5c5c4ccc4ccccc45)nc4ccccc34)cc2)cc1 ; c1ccc(N(c2ccccc2)c2ccc3c(c2)nc(-c2ccccc2-c2nc4ccccc4n2-c2ccccc2)c2ccc4ccccc4c23)cc1 ; c1ccc(N(c2ccccc2)c2ccc3c(c2)nc(-c2cccc(-c4ccc5ccc6cccnc6c5n4)c2)c2ccc4ccccc4c23)cc1]

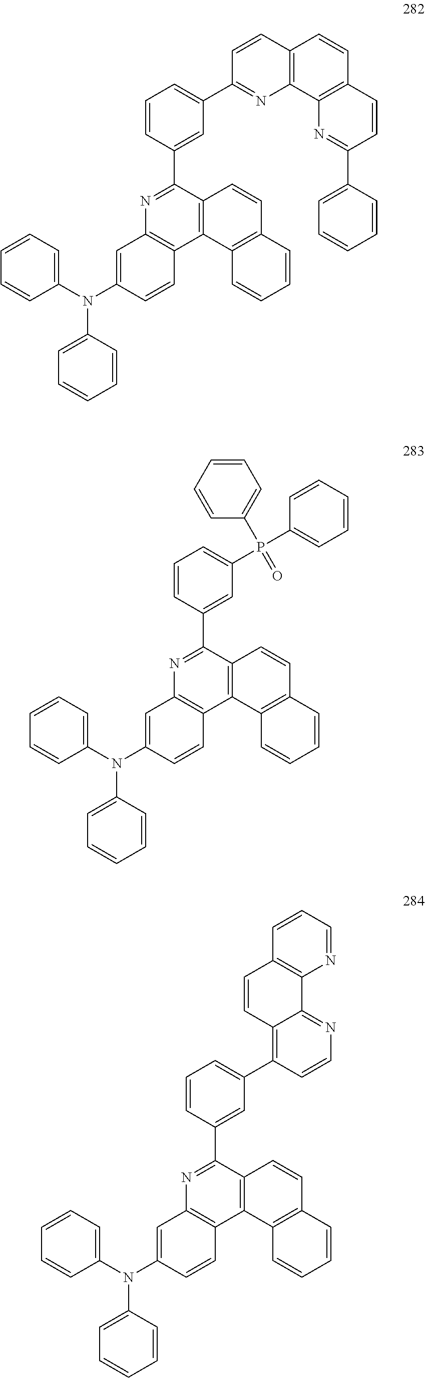 [SMILES: c1ccc(-c2ccc3ccc4ccc(-c5cccc(-c6nc7cc(N(c8ccccc8)c8ccccc8)ccc7c7c6ccc6ccccc67)c5)nc4c3n2)cc1 ; O=P(c1ccccc1)(c1ccccc1)c1cccc(-c2nc3cc(N(c4ccccc4)c4ccccc4)ccc3c3c2ccc2ccccc23)c1 ; c1ccc(N(c2ccccc2)c2ccc3c(c2)nc(-c2cccc(-c4ccnc5c4ccc4cccnc45)c2)c2ccc4ccccc4c23)cc1]

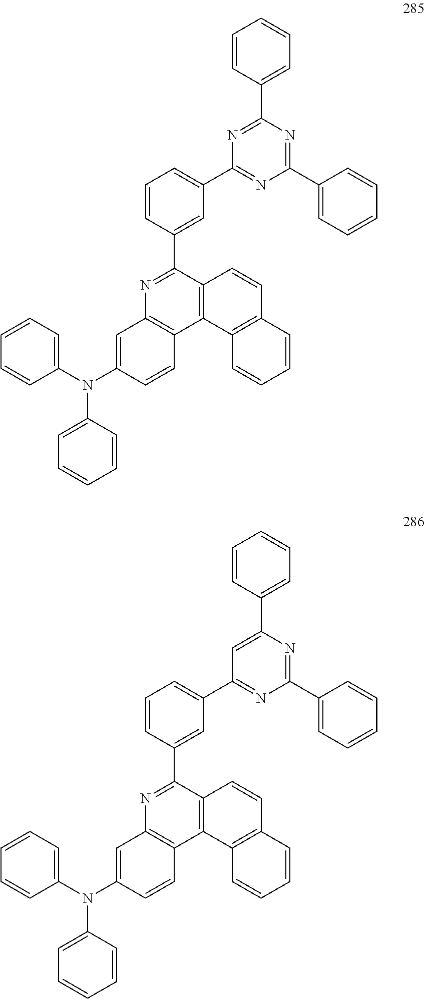 [SMILES: c1ccc(-c2nc(-c3ccccc3)nc(-c3cccc(-c4nc5cc(N(c6ccccc6)c6ccccc6)ccc5c5c4ccc4ccccc45)c3)n2)cc1 ; c1ccc(-c2cc(-c3cccc(-c4nc5cc(N(c6ccccc6)c6ccccc6)ccc5c5c4ccc4ccccc45)c3)nc(-c3ccccc3)n2)cc1]

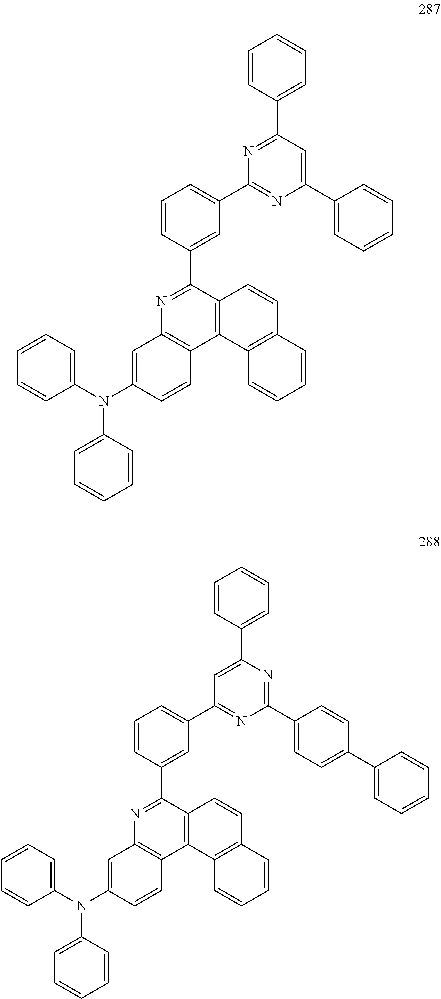 [SMILES: c1ccc(-c2cc(-c3ccccc3)nc(-c3cccc(-c4nc5cc(N(c6ccccc6)c6ccccc6)ccc5c5c4ccc4ccccc45)c3)n2)cc1 ; c1ccc(-c2ccc(-c3nc(-c4ccccc4)cc(-c4cccc(-c5nc6cc(N(c7ccccc7)c7ccccc7)ccc6c6c5ccc5ccccc56)c4)n3)cc2)cc1]

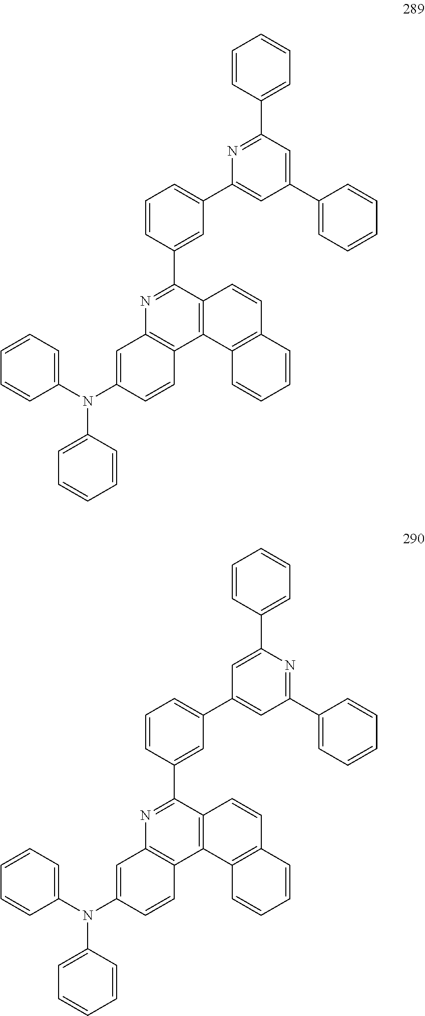 [SMILES: c1ccc(-c2cc(-c3ccccc3)nc(-c3cccc(-c4nc5cc(N(c6ccccc6)c6ccccc6)ccc5c5c4ccc4ccccc45)c3)c2)cc1 ; c1ccc(-c2cc(-c3cccc(-c4nc5cc(N(c6ccccc6)c6ccccc6)ccc5c5c4ccc4ccccc45)c3)cc(-c3ccccc3)n2)cc1]

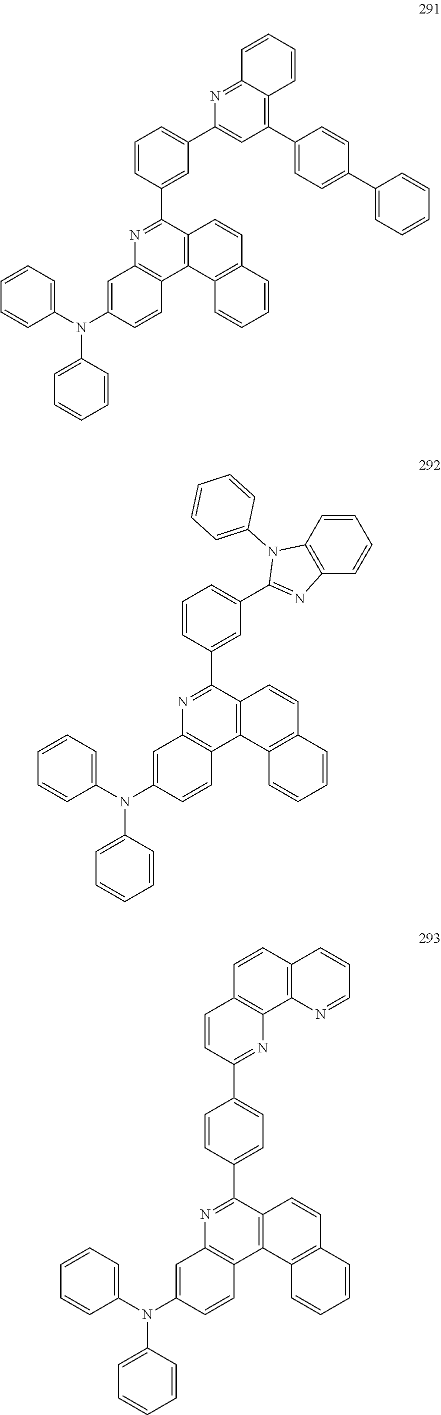 [SMILES: c1ccc(-c2ccc(-c3cc(-c4cccc(-c5nc6cc(N(c7ccccc7)c7ccccc7)ccc6c6c5ccc5ccccc56)c4)nc4ccccc34)cc2)cc1 ; c1ccc(N(c2ccccc2)c2ccc3c(c2)nc(-c2cccc(-c4nc5ccccc5n4-c4ccccc4)c2)c2ccc4ccccc4c23)cc1 ; c1ccc(N(c2ccccc2)c2ccc3c(c2)nc(-c2ccc(-c4ccc5ccc6cccnc6c5n4)cc2)c2ccc4ccccc4c23)cc1]

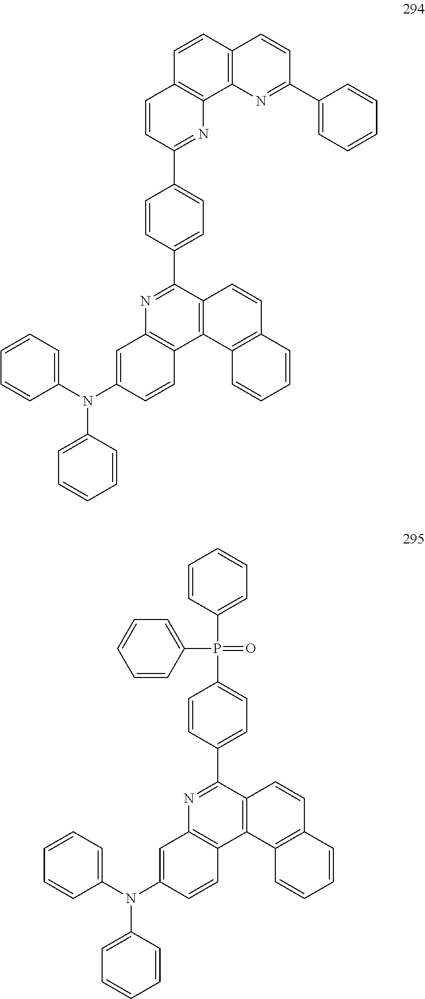 [SMILES: c1ccc(-c2ccc3ccc4ccc(-c5ccc(-c6nc7cc(N(c8ccccc8)c8ccccc8)ccc7c7c6ccc6ccccc67)cc5)nc4c3n2)cc1 ; O=P(c1ccccc1)(c1ccccc1)c1ccc(-c2nc3cc(N(c4ccccc4)c4ccccc4)ccc3c3c2ccc2ccccc23)cc1]

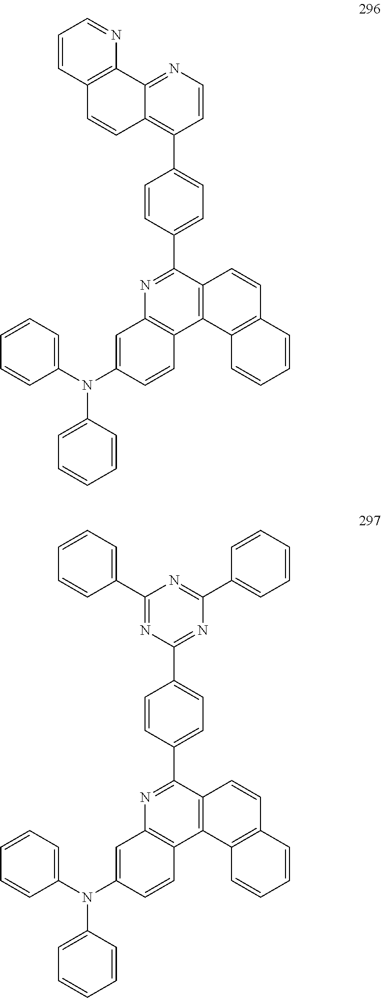

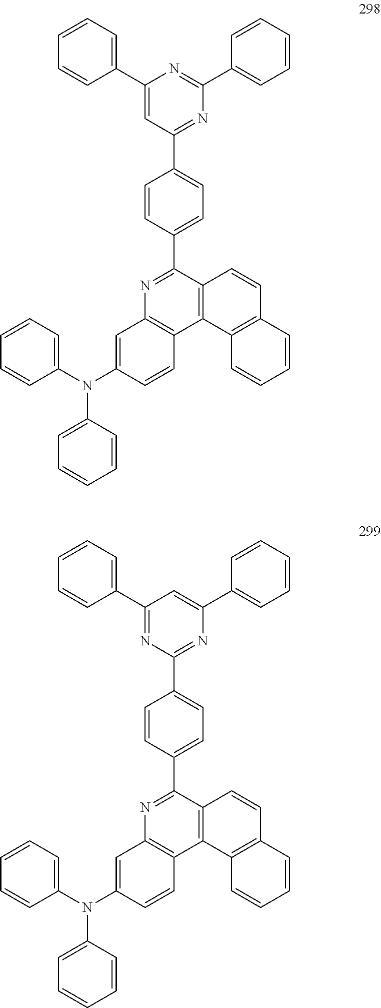 [SMILES: c1ccc(-c2cc(-c3ccc(-c4nc5cc(N(c6ccccc6)c6ccccc6)ccc5c5c4ccc4ccccc45)cc3)nc(-c3ccccc3)n2)cc1 ; c1ccc(-c2cc(-c3ccccc3)nc(-c3ccc(-c4nc5cc(N(c6ccccc6)c6ccccc6)ccc5c5c4ccc4ccccc45)cc3)n2)cc1]

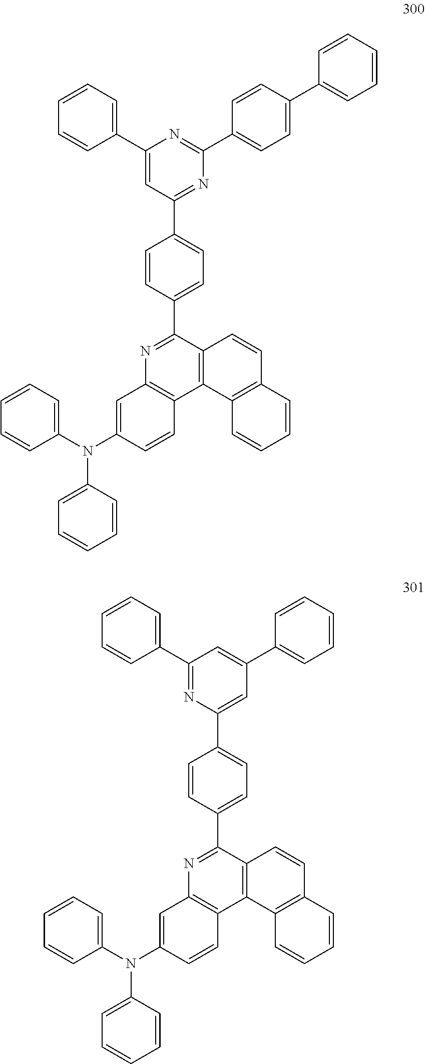 [SMILES: c1ccc(-c2ccc(-c3nc(-c4ccccc4)cc(-c4ccc(-c5nc6cc(N(c7ccccc7)c7ccccc7)ccc6c6c5ccc5ccccc56)cc4)n3)cc2)cc1 ; c1ccc(-c2cc(-c3ccccc3)nc(-c3ccc(-c4nc5cc(N(c6ccccc6)c6ccccc6)ccc5c5c4ccc4ccccc45)cc3)c2)cc1]

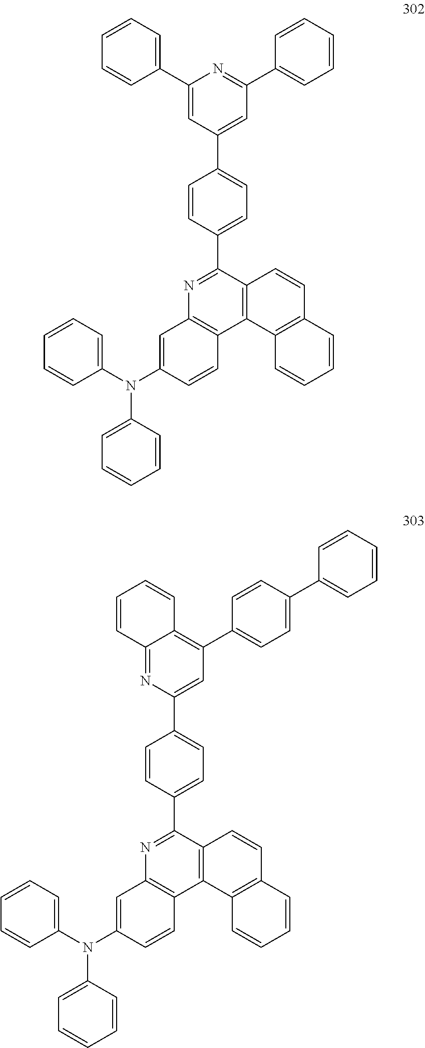 [SMILES: c1ccc(-c2cc(-c3ccc(-c4nc5cc(N(c6ccccc6)c6ccccc6)ccc5c5c4ccc4ccccc45)cc3)cc(-c3ccccc3)n2)cc1 ; c1ccc(-c2ccc(-c3cc(-c4ccc(-c5nc6cc(N(c7ccccc7)c7ccccc7)ccc6c6c5ccc5ccccc56)cc4)nc4ccccc34)cc2)cc1]

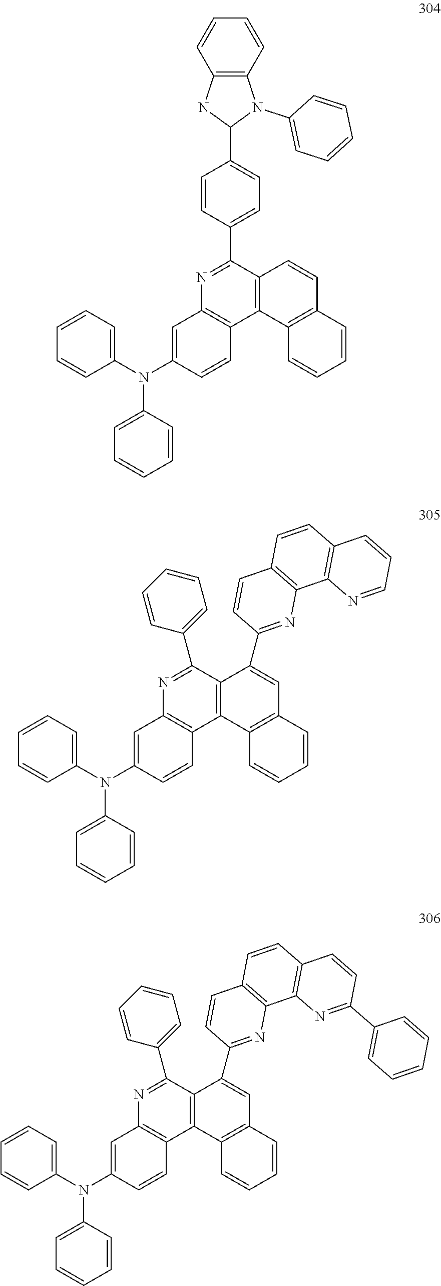 [SMILES: c1ccc(N(c2ccccc2)c2ccc3c(c2)nc(-c2ccc(C4Nc5ccccc5N4c4ccccc4)cc2)c2ccc4ccccc4c23)cc1 ; c1ccc(-c2nc3cc(N(c4ccccc4)c4ccccc4)ccc3c3c2c(-c2ccc4ccc5cccnc5c4n2)cc2ccccc23)cc1 ; c1ccc(-c2ccc3ccc4ccc(-c5cc6ccccc6c6c5c(-c5ccccc5)nc5cc(N(c7ccccc7)c7ccccc7)ccc56)nc4c3n2)cc1]

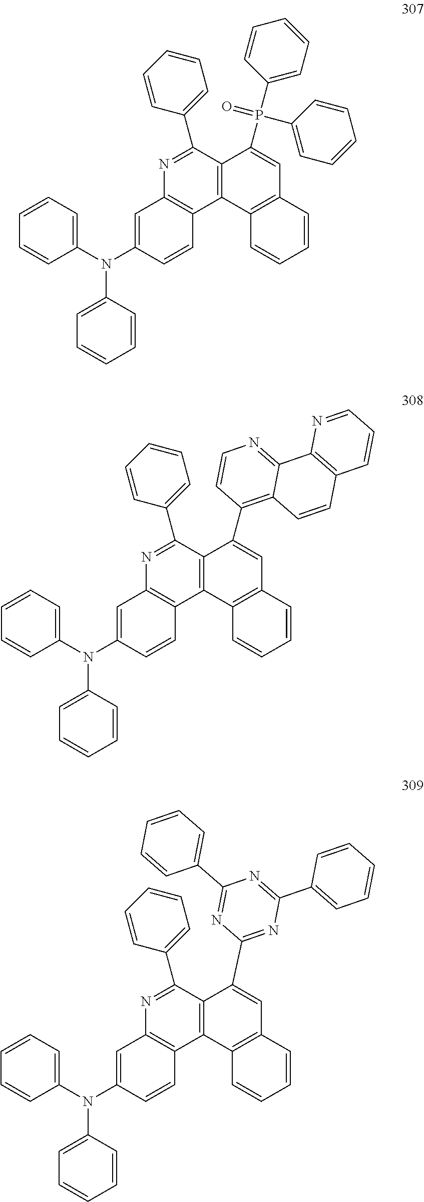 [SMILES: O=P(c1ccccc1)(c1ccccc1)c1cc2ccccc2c2c1c(-c1ccccc1)nc1cc(N(c3ccccc3)c3ccccc3)ccc12 ; c1ccc(-c2nc3cc(N(c4ccccc4)c4ccccc4)ccc3c3c2c(-c2ccnc4c2ccc2cccnc24)cc2ccccc23)cc1 ; c1ccc(-c2nc(-c3ccccc3)nc(-c3cc4ccccc4c4c3c(-c3ccccc3)nc3cc(N(c5ccccc5)c5ccccc5)ccc34)n2)cc1]

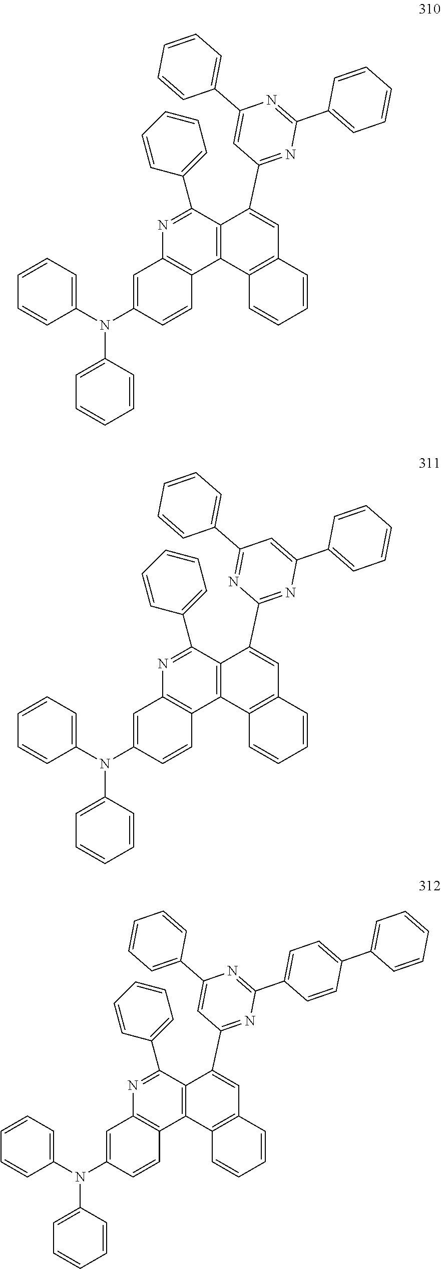 [SMILES: c1ccc(-c2cc(-c3cc4ccccc4c4c3c(-c3ccccc3)nc3cc(N(c5ccccc5)c5ccccc5)ccc34)nc(-c3ccccc3)n2)cc1 ; c1ccc(-c2cc(-c3ccccc3)nc(-c3cc4ccccc4c4c3c(-c3ccccc3)nc3cc(N(c5ccccc5)c5ccccc5)ccc34)n2)cc1 ; c1ccc(-c2ccc(-c3nc(-c4ccccc4)cc(-c4cc5ccccc5c5c4c(-c4ccccc4)nc4cc(N(c6ccccc6)c6ccccc6)ccc45)n3)cc2)cc1]

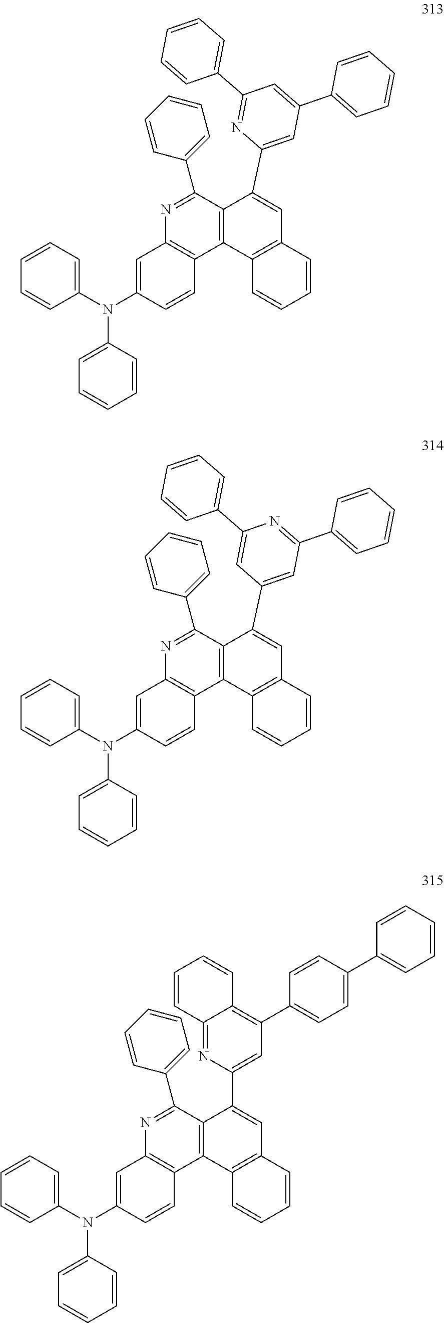 [SMILES: c1ccc(-c2cc(-c3ccccc3)nc(-c3cc4ccccc4c4c3c(-c3ccccc3)nc3cc(N(c5ccccc5)c5ccccc5)ccc34)c2)cc1 ; c1ccc(-c2cc(-c3cc4ccccc4c4c3c(-c3ccccc3)nc3cc(N(c5ccccc5)c5ccccc5)ccc34)cc(-c3ccccc3)n2)cc1 ; c1ccc(-c2ccc(-c3cc(-c4cc5ccccc5c5c4c(-c4ccccc4)nc4cc(N(c6ccccc6)c6ccccc6)ccc45)nc4ccccc34)cc2)cc1]

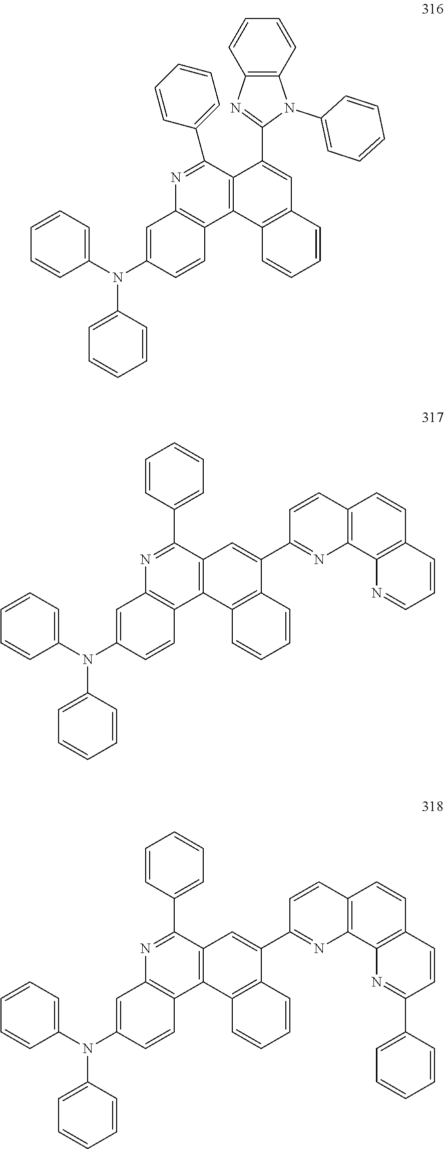 [SMILES: c1ccc(-c2nc3cc(N(c4ccccc4)c4ccccc4)ccc3c3c2c(-c2nc4ccccc4n2-c2ccccc2)cc2ccccc23)cc1 ; c1ccc(-c2nc3cc(N(c4ccccc4)c4ccccc4)ccc3c3c2cc(-c2ccc4ccc5cccnc5c4n2)c2ccccc23)cc1 ; c1ccc(-c2ccc3ccc4ccc(-c5cc6c(-c7ccccc7)nc7cc(N(c8ccccc8)c8ccccc8)ccc7c6c6ccccc56)nc4c3n2)cc1]

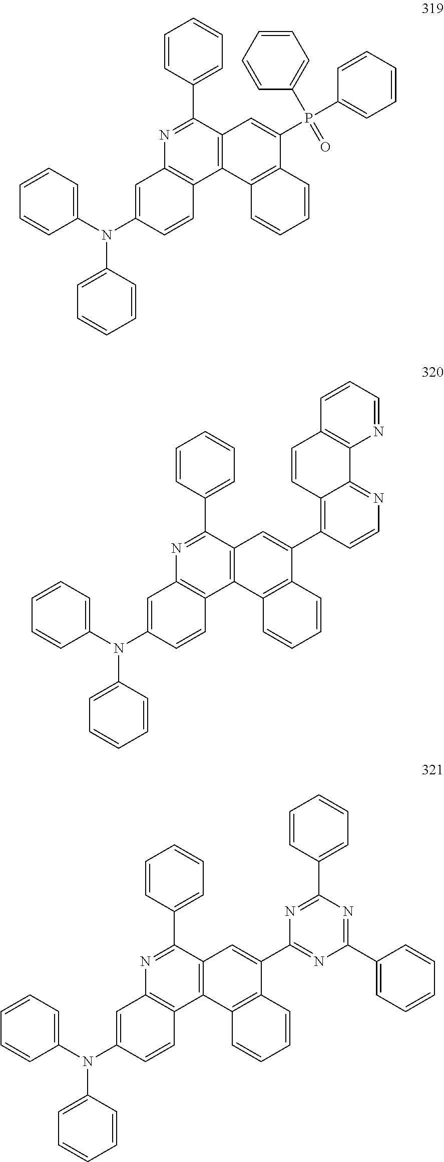 [SMILES: O=P(c1ccccc1)(c1ccccc1)c1cc2c(-c3ccccc3)nc3cc(N(c4ccccc4)c4ccccc4)ccc3c2c2ccccc12 ; c1ccc(-c2nc3cc(N(c4ccccc4)c4ccccc4)ccc3c3c2cc(-c2ccnc4c2ccc2cccnc24)c2ccccc23)cc1 ; c1ccc(-c2nc(-c3ccccc3)nc(-c3cc4c(-c5ccccc5)nc5cc(N(c6ccccc6)c6ccccc6)ccc5c4c4ccccc34)n2)cc1]

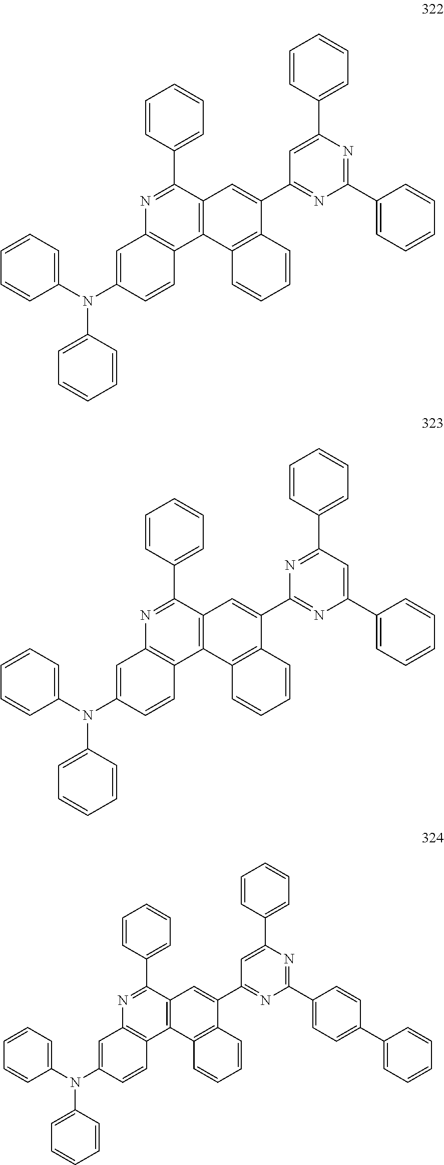 [SMILES: c1ccc(-c2cc(-c3cc4c(-c5ccccc5)nc5cc(N(c6ccccc6)c6ccccc6)ccc5c4c4ccccc34)nc(-c3ccccc3)n2)cc1 ; c1ccc(-c2cc(-c3ccccc3)nc(-c3cc4c(-c5ccccc5)nc5cc(N(c6ccccc6)c6ccccc6)ccc5c4c4ccccc34)n2)cc1 ; c1ccc(-c2ccc(-c3nc(-c4ccccc4)cc(-c4cc5c(-c6ccccc6)nc6cc(N(c7ccccc7)c7ccccc7)ccc6c5c5ccccc45)n3)cc2)cc1]

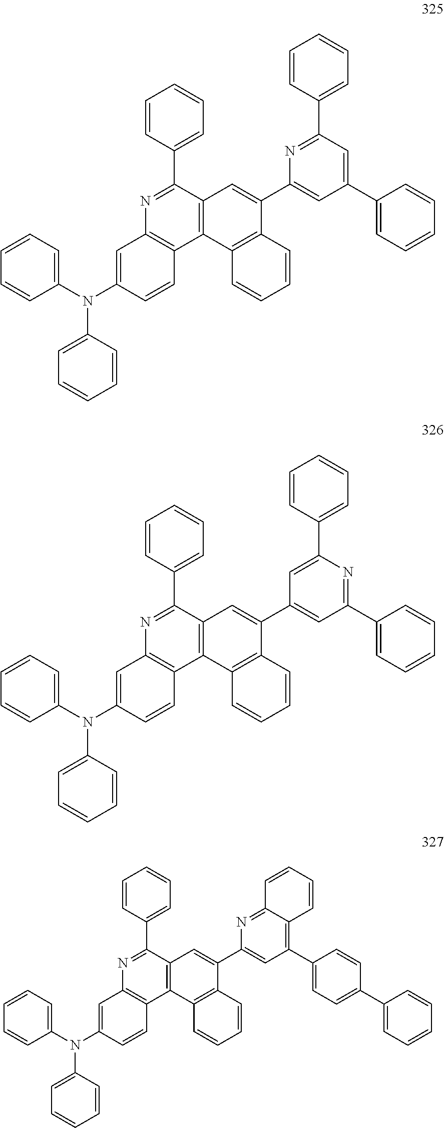 [SMILES: c1ccc(-c2cc(-c3ccccc3)nc(-c3cc4c(-c5ccccc5)nc5cc(N(c6ccccc6)c6ccccc6)ccc5c4c4ccccc34)c2)cc1 ; c1ccc(-c2cc(-c3cc4c(-c5ccccc5)nc5cc(N(c6ccccc6)c6ccccc6)ccc5c4c4ccccc34)cc(-c3ccccc3)n2)cc1 ; c1ccc(-c2ccc(-c3cc(-c4cc5c(-c6ccccc6)nc6cc(N(c7ccccc7)c7ccccc7)ccc6c5c5ccccc45)nc4ccccc34)cc2)cc1]

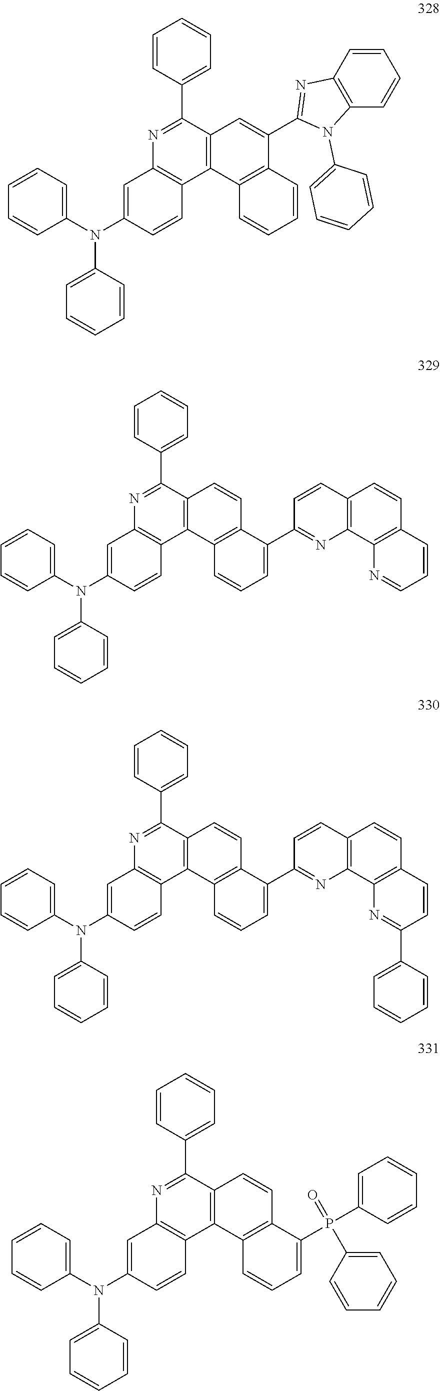 [SMILES: c1ccc(-c2nc3cc(N(c4ccccc4)c4ccccc4)ccc3c3c2cc(-c2nc4ccccc4n2-c2ccccc2)c2ccccc23)cc1 ; c1ccc(-c2nc3cc(N(c4ccccc4)c4ccccc4)ccc3c3c2ccc2c(-c4ccc5ccc6cccnc6c5n4)cccc23)cc1 ; c1ccc(-c2ccc3ccc4ccc(-c5cccc6c5ccc5c(-c7ccccc7)nc7cc(N(c8ccccc8)c8ccccc8)ccc7c56)nc4c3n2)cc1 ; O=P(c1ccccc1)(c1ccccc1)c1cccc2c1ccc1c(-c3ccccc3)nc3cc(N(c4ccccc4)c4ccccc4)ccc3c12]

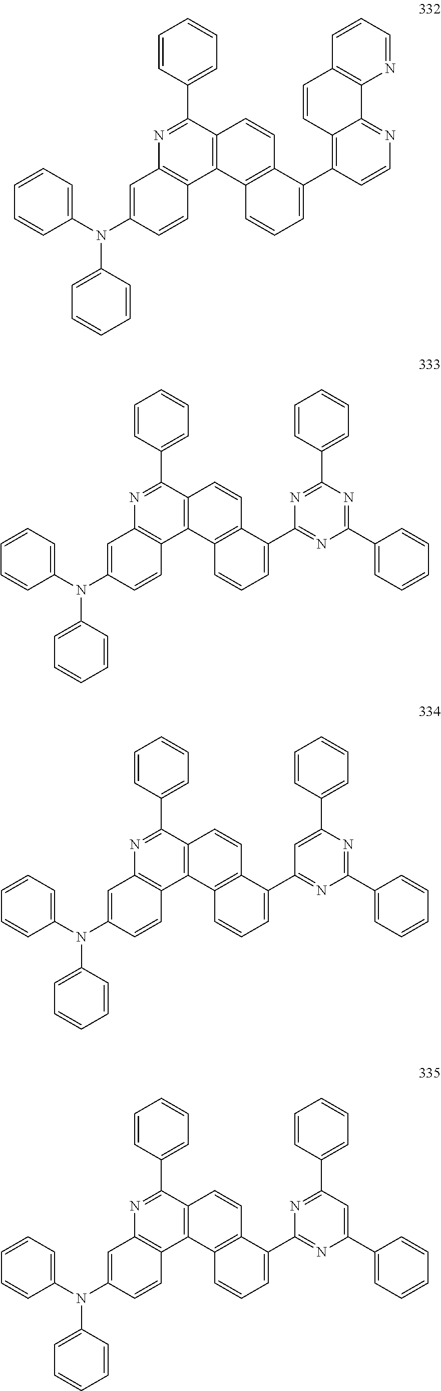 [SMILES: c1ccc(-c2nc3cc(N(c4ccccc4)c4ccccc4)ccc3c3c2ccc2c(-c4ccnc5c4ccc4cccnc45)cccc23)cc1 ; c1ccc(-c2nc(-c3ccccc3)nc(-c3cccc4c3ccc3c(-c5ccccc5)nc5cc(N(c6ccccc6)c6ccccc6)ccc5c34)n2)cc1 ; c1ccc(-c2cc(-c3cccc4c3ccc3c(-c5ccccc5)nc5cc(N(c6ccccc6)c6ccccc6)ccc5c34)nc(-c3ccccc3)n2)cc1 ; c1ccc(-c2cc(-c3ccccc3)nc(-c3cccc4c3ccc3c(-c5ccccc5)nc5cc(N(c6ccccc6)c6ccccc6)ccc5c34)n2)cc1]

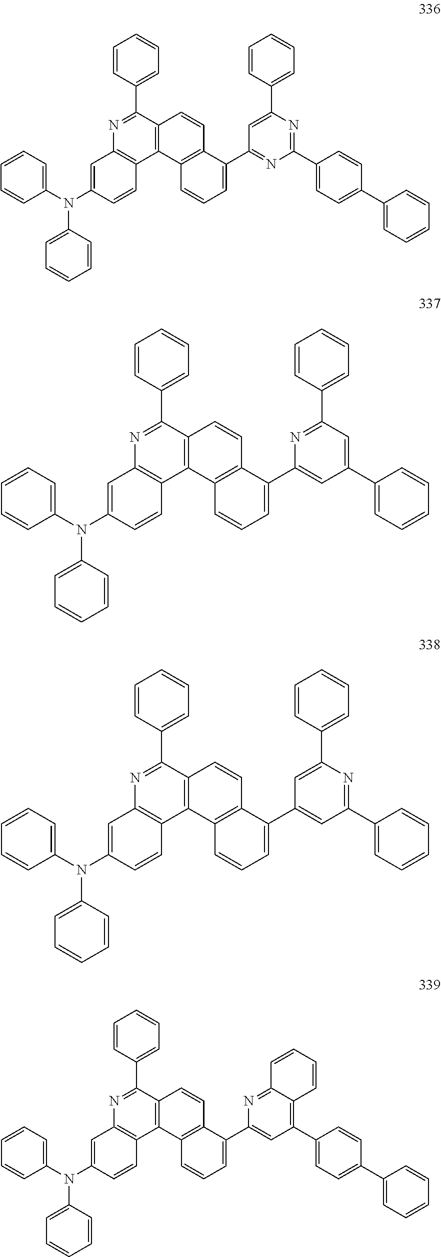 [SMILES: c1ccc(-c2ccc(-c3nc(-c4ccccc4)cc(-c4cccc5c4ccc4c(-c6ccccc6)nc6cc(N(c7ccccc7)c7ccccc7)ccc6c45)n3)cc2)cc1 ; c1ccc(-c2cc(-c3ccccc3)nc(-c3cccc4c3ccc3c(-c5ccccc5)nc5cc(N(c6ccccc6)c6ccccc6)ccc5c34)c2)cc1 ; c1ccc(-c2cc(-c3cccc4c3ccc3c(-c5ccccc5)nc5cc(N(c6ccccc6)c6ccccc6)ccc5c34)cc(-c3ccccc3)n2)cc1 ; c1ccc(-c2ccc(-c3cc(-c4cccc5c4ccc4c(-c6ccccc6)nc6cc(N(c7ccccc7)c7ccccc7)ccc6c45)nc4ccccc34)cc2)cc1]

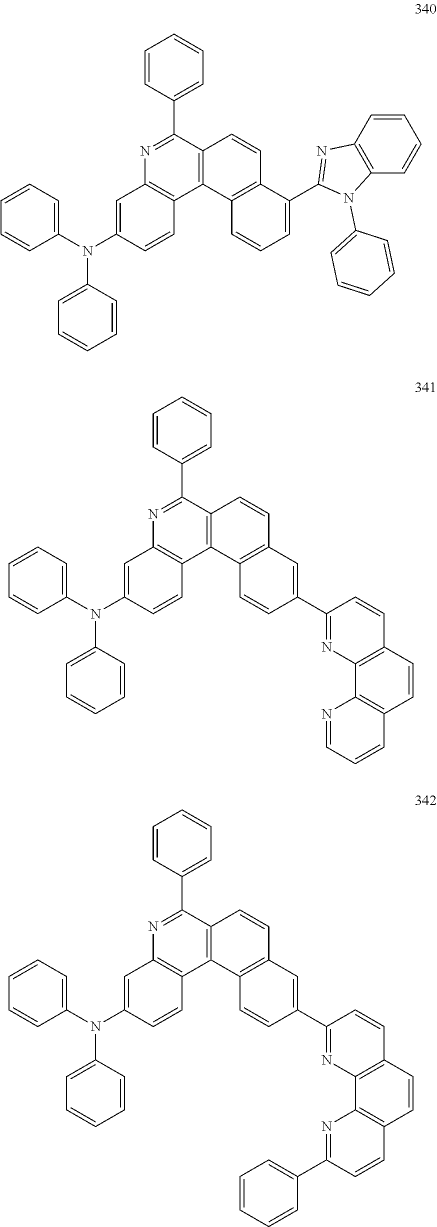 [SMILES: c1ccc(-c2nc3cc(N(c4ccccc4)c4ccccc4)ccc3c3c2ccc2c(-c4nc5ccccc5n4-c4ccccc4)cccc23)cc1 ; c1ccc(-c2nc3cc(N(c4ccccc4)c4ccccc4)ccc3c3c2ccc2cc(-c4ccc5ccc6cccnc6c5n4)ccc23)cc1 ; c1ccc(-c2ccc3ccc4ccc(-c5ccc6c(ccc7c(-c8ccccc8)nc8cc(N(c9ccccc9)c9ccccc9)ccc8c76)c5)nc4c3n2)cc1]

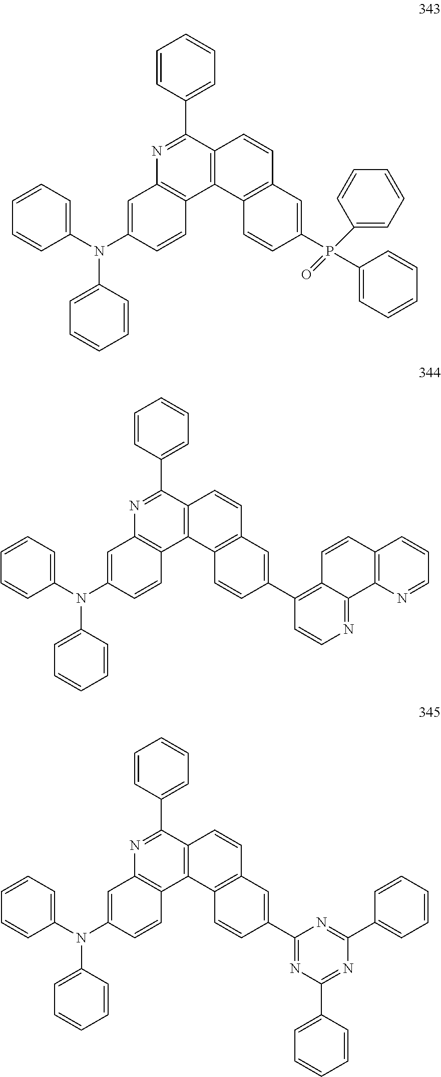 [SMILES: O=P(c1ccccc1)(c1ccccc1)c1ccc2c(ccc3c(-c4ccccc4)nc4cc(N(c5ccccc5)c5ccccc5)ccc4c32)c1 ; c1ccc(-c2nc3cc(N(c4ccccc4)c4ccccc4)ccc3c3c2ccc2cc(-c4ccnc5c4ccc4cccnc45)ccc23)cc1 ; c1ccc(-c2nc(-c3ccccc3)nc(-c3ccc4c(ccc5c(-c6ccccc6)nc6cc(N(c7ccccc7)c7ccccc7)ccc6c54)c3)n2)cc1]

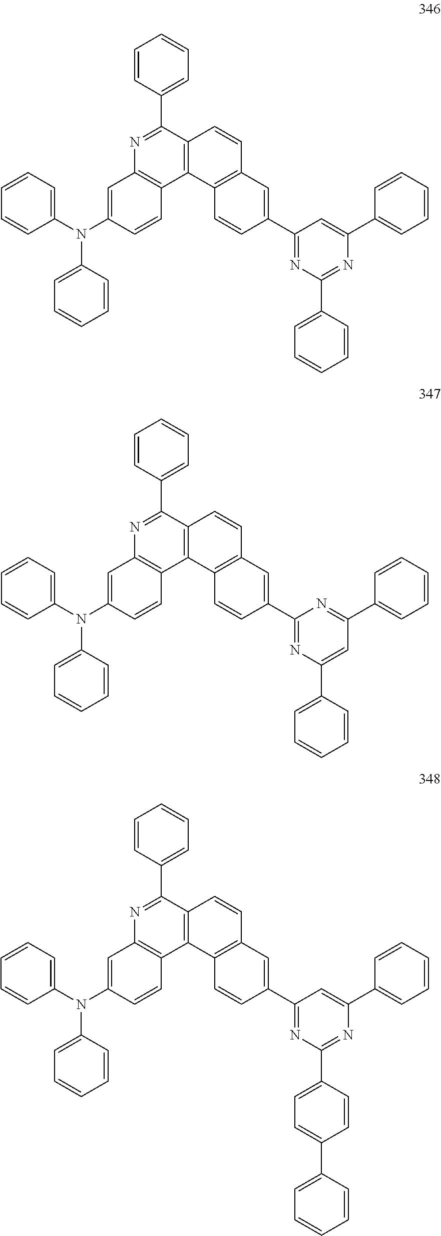 [SMILES: c1ccc(-c2cc(-c3ccc4c(ccc5c(-c6ccccc6)nc6cc(N(c7ccccc7)c7ccccc7)ccc6c54)c3)nc(-c3ccccc3)n2)cc1 ; c1ccc(-c2cc(-c3ccccc3)nc(-c3ccc4c(ccc5c(-c6ccccc6)nc6cc(N(c7ccccc7)c7ccccc7)ccc6c54)c3)n2)cc1 ; c1ccc(-c2ccc(-c3nc(-c4ccccc4)cc(-c4ccc5c(ccc6c(-c7ccccc7)nc7cc(N(c8ccccc8)c8ccccc8)ccc7c65)c4)n3)cc2)cc1]

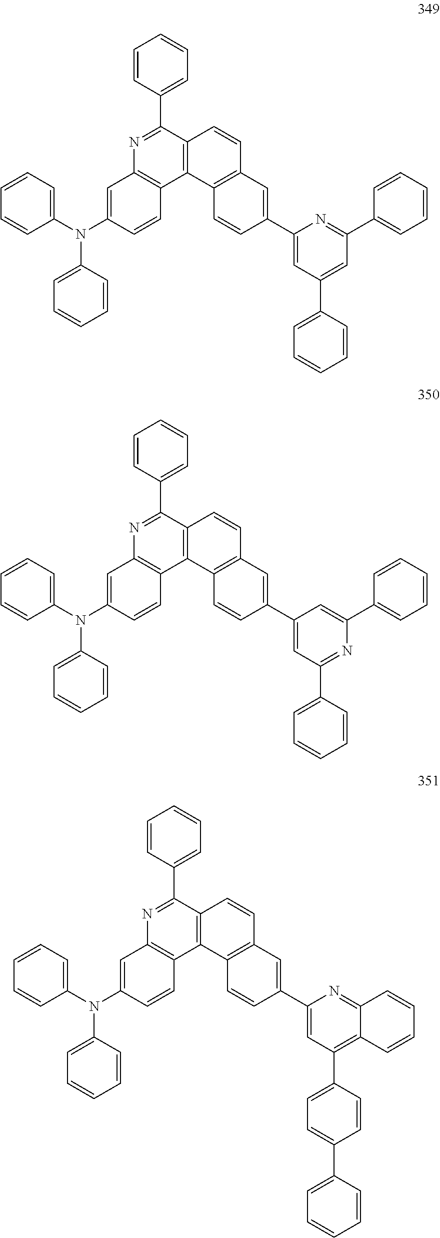 [SMILES: c1ccc(-c2cc(-c3ccccc3)nc(-c3ccc4c(ccc5c(-c6ccccc6)nc6cc(N(c7ccccc7)c7ccccc7)ccc6c54)c3)c2)cc1 ; c1ccc(-c2cc(-c3ccc4c(ccc5c(-c6ccccc6)nc6cc(N(c7ccccc7)c7ccccc7)ccc6c54)c3)cc(-c3ccccc3)n2)cc1 ; c1ccc(-c2ccc(-c3cc(-c4ccc5c(ccc6c(-c7ccccc7)nc7cc(N(c8ccccc8)c8ccccc8)ccc7c65)c4)nc4ccccc34)cc2)cc1]

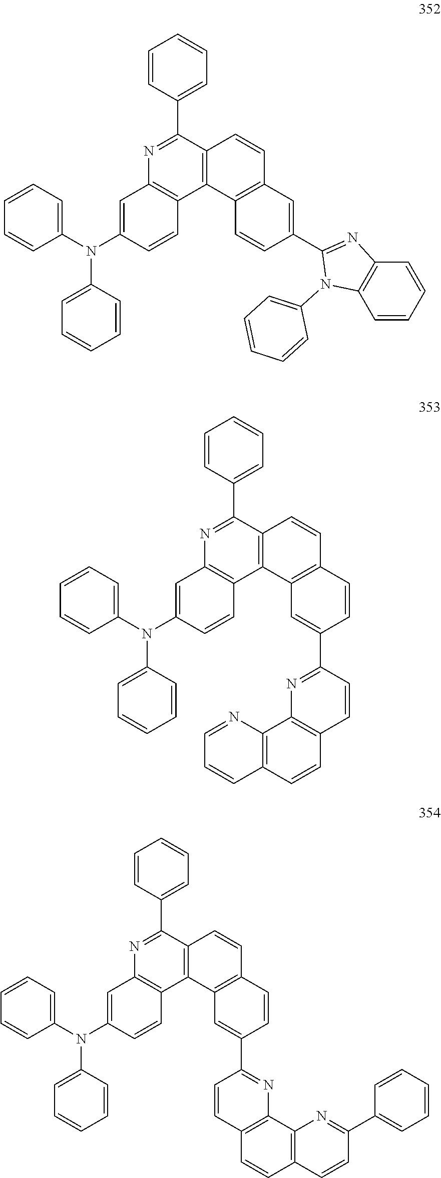 [SMILES: c1ccc(-c2nc3cc(N(c4ccccc4)c4ccccc4)ccc3c3c2ccc2cc(-c4nc5ccccc5n4-c4ccccc4)ccc23)cc1 ; c1ccc(-c2nc3cc(N(c4ccccc4)c4ccccc4)ccc3c3c2ccc2ccc(-c4ccc5ccc6cccnc6c5n4)cc23)cc1 ; c1ccc(-c2ccc3ccc4ccc(-c5ccc6ccc7c(-c8ccccc8)nc8cc(N(c9ccccc9)c9ccccc9)ccc8c7c6c5)nc4c3n2)cc1]

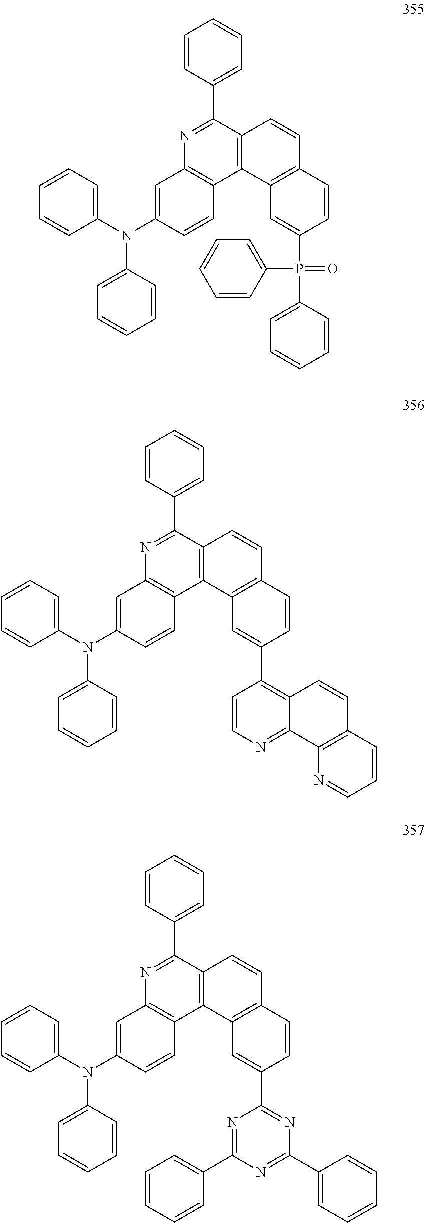 [SMILES: O=P(c1ccccc1)(c1ccccc1)c1ccc2ccc3c(-c4ccccc4)nc4cc(N(c5ccccc5)c5ccccc5)ccc4c3c2c1 ; c1ccc(-c2nc3cc(N(c4ccccc4)c4ccccc4)ccc3c3c2ccc2ccc(-c4ccnc5c4ccc4cccnc45)cc23)cc1 ; c1ccc(-c2nc(-c3ccccc3)nc(-c3ccc4ccc5c(-c6ccccc6)nc6cc(N(c7ccccc7)c7ccccc7)ccc6c5c4c3)n2)cc1]

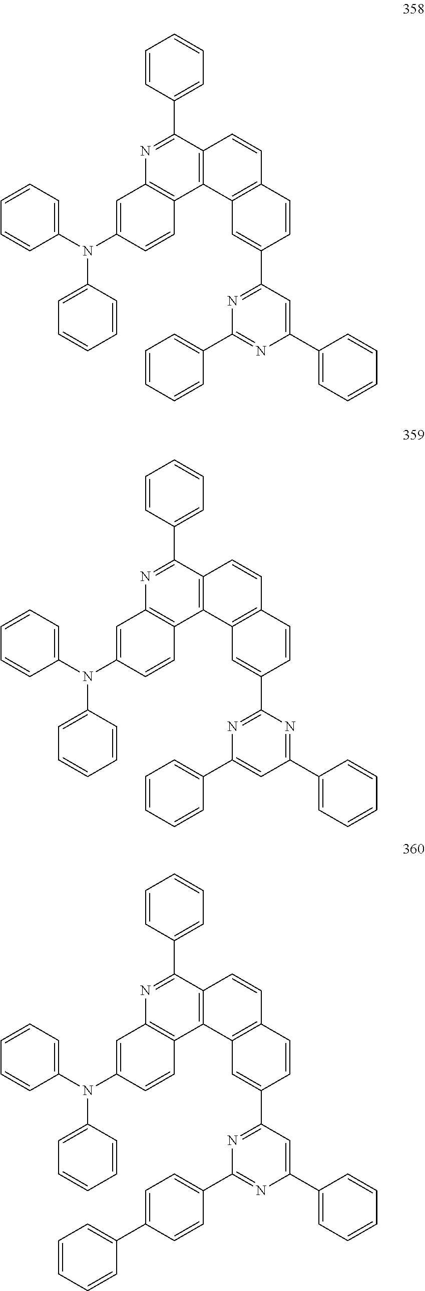 [SMILES: c1ccc(-c2cc(-c3ccc4ccc5c(-c6ccccc6)nc6cc(N(c7ccccc7)c7ccccc7)ccc6c5c4c3)nc(-c3ccccc3)n2)cc1 ; c1ccc(-c2cc(-c3ccccc3)nc(-c3ccc4ccc5c(-c6ccccc6)nc6cc(N(c7ccccc7)c7ccccc7)ccc6c5c4c3)n2)cc1 ; c1ccc(-c2ccc(-c3nc(-c4ccccc4)cc(-c4ccc5ccc6c(-c7ccccc7)nc7cc(N(c8ccccc8)c8ccccc8)ccc7c6c5c4)n3)cc2)cc1]

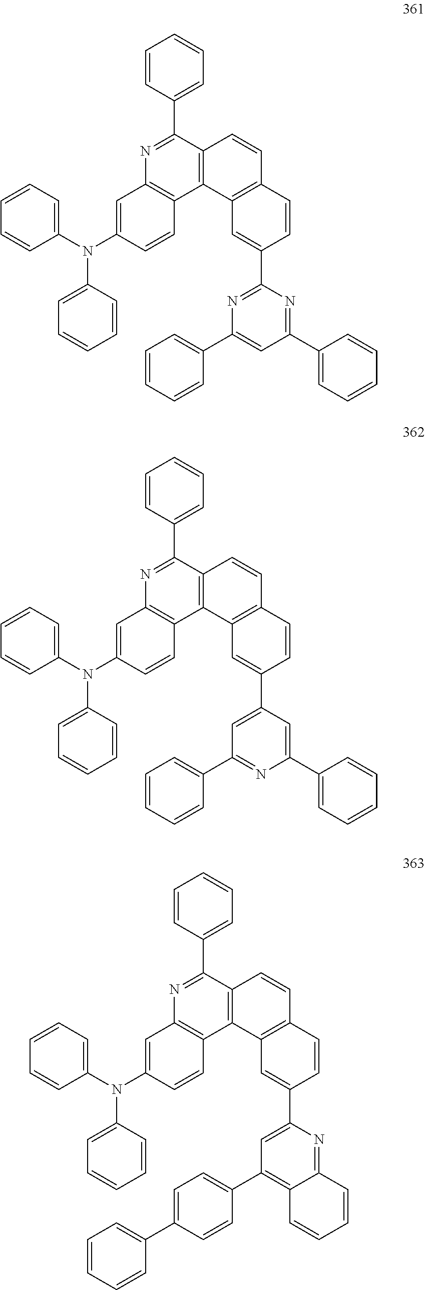 [SMILES: c1ccc(-c2cc(-c3ccccc3)nc(-c3ccc4ccc5c(-c6ccccc6)nc6cc(N(c7ccccc7)c7ccccc7)ccc6c5c4c3)n2)cc1 ; c1ccc(-c2cc(-c3ccc4ccc5c(-c6ccccc6)nc6cc(N(c7ccccc7)c7ccccc7)ccc6c5c4c3)cc(-c3ccccc3)n2)cc1 ; c1ccc(-c2ccc(-c3cc(-c4ccc5ccc6c(-c7ccccc7)nc7cc(N(c8ccccc8)c8ccccc8)ccc7c6c5c4)nc4ccccc34)cc2)cc1]

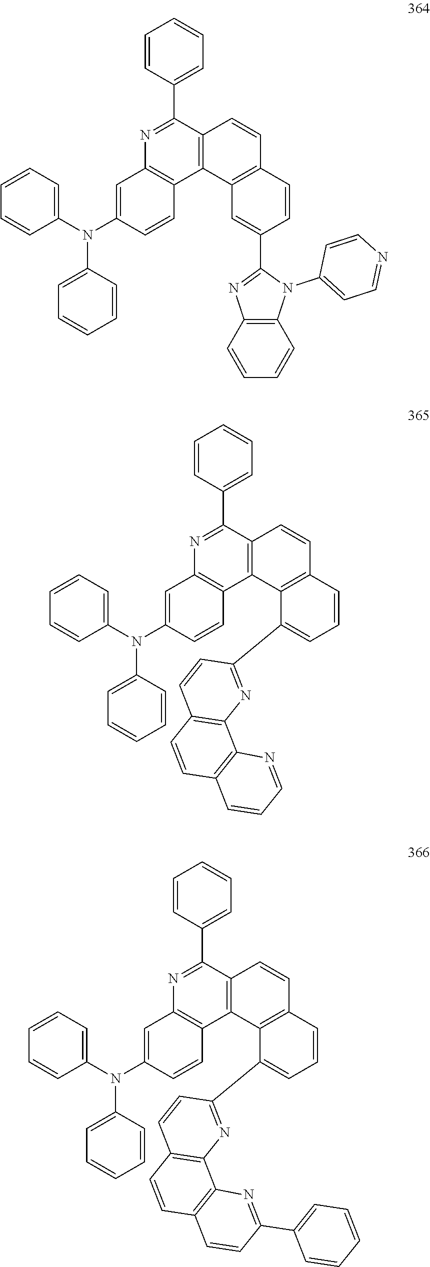 [SMILES: c1ccc(-c2nc3cc(N(c4ccccc4)c4ccccc4)ccc3c3c2ccc2ccc(-c4nc5ccccc5n4-c4ccncc4)cc23)cc1 ; c1ccc(-c2nc3cc(N(c4ccccc4)c4ccccc4)ccc3c3c2ccc2cccc(-c4ccc5ccc6cccnc6c5n4)c23)cc1 ; c1ccc(-c2ccc3ccc4ccc(-c5cccc6ccc7c(-c8ccccc8)nc8cc(N(c9ccccc9)c9ccccc9)ccc8c7c56)nc4c3n2)cc1]

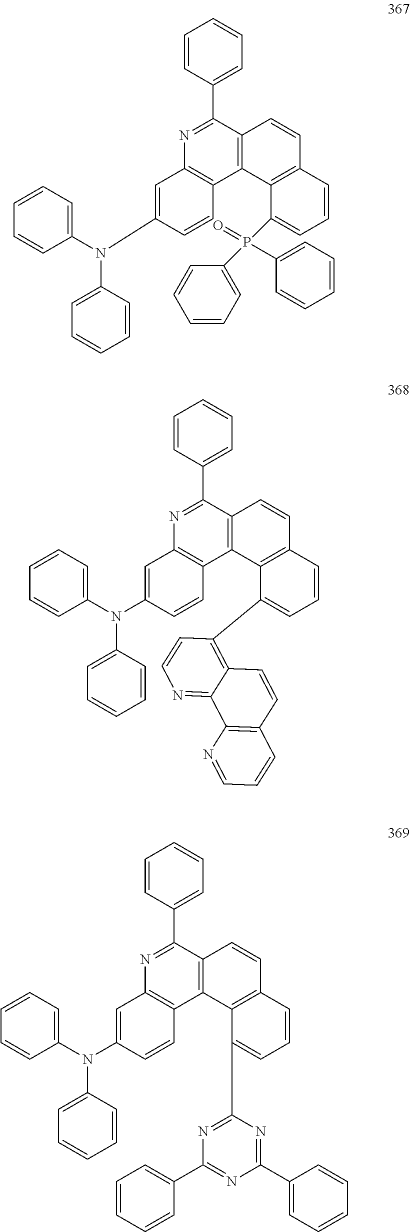 [SMILES: O=P(c1ccccc1)(c1ccccc1)c1cccc2ccc3c(-c4ccccc4)nc4cc(N(c5ccccc5)c5ccccc5)ccc4c3c12 ; c1ccc(-c2nc3cc(N(c4ccccc4)c4ccccc4)ccc3c3c2ccc2cccc(-c4ccnc5c4ccc4cccnc45)c23)cc1 ; c1ccc(-c2nc(-c3ccccc3)nc(-c3cccc4ccc5c(-c6ccccc6)nc6cc(N(c7ccccc7)c7ccccc7)ccc6c5c34)n2)cc1]

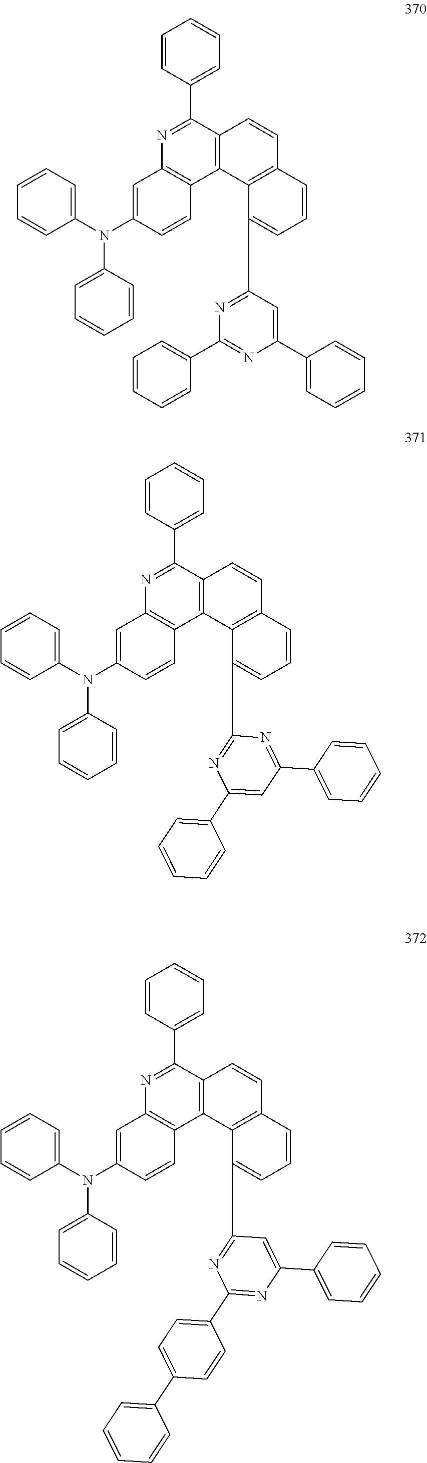 [SMILES: c1ccc(-c2cc(-c3cccc4ccc5c(-c6ccccc6)nc6cc(N(c7ccccc7)c7ccccc7)ccc6c5c34)nc(-c3ccccc3)n2)cc1 ; c1ccc(-c2cc(-c3ccccc3)nc(-c3cccc4ccc5c(-c6ccccc6)nc6cc(N(c7ccccc7)c7ccccc7)ccc6c5c34)n2)cc1 ; c1ccc(-c2ccc(-c3nc(-c4ccccc4)cc(-c4cccc5ccc6c(-c7ccccc7)nc7cc(N(c8ccccc8)c8ccccc8)ccc7c6c45)n3)cc2)cc1]

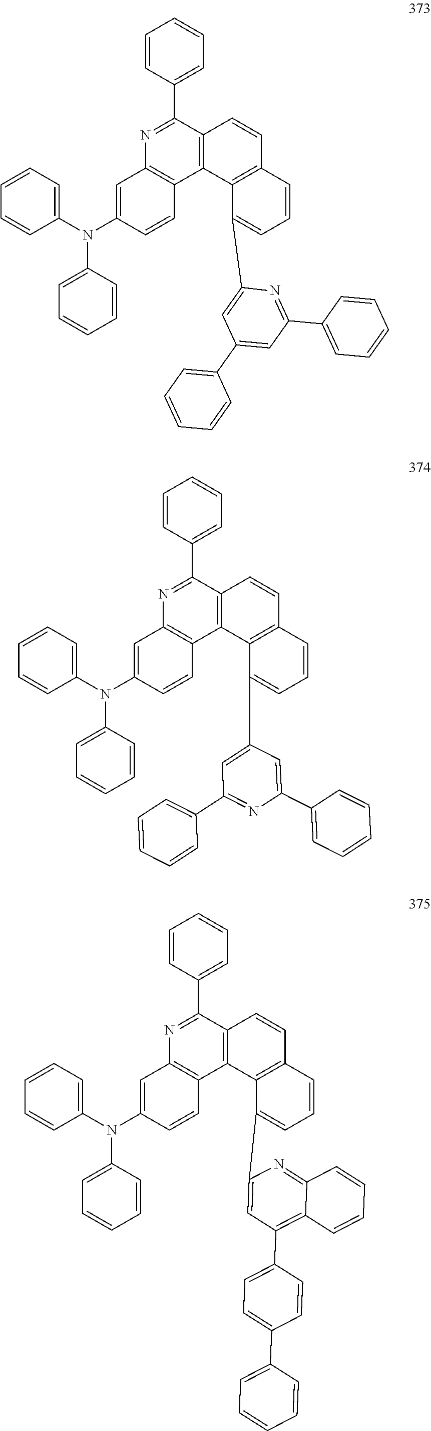 [SMILES: c1ccc(-c2cc(-c3ccccc3)nc(-c3cccc4ccc5c(-c6ccccc6)nc6cc(N(c7ccccc7)c7ccccc7)ccc6c5c34)c2)cc1 ; c1ccc(-c2cc(-c3cccc4ccc5c(-c6ccccc6)nc6cc(N(c7ccccc7)c7ccccc7)ccc6c5c34)cc(-c3ccccc3)n2)cc1 ; c1ccc(-c2ccc(-c3cc(-c4cccc5ccc6c(-c7ccccc7)nc7cc(N(c8ccccc8)c8ccccc8)ccc7c6c45)nc4ccccc34)cc2)cc1]

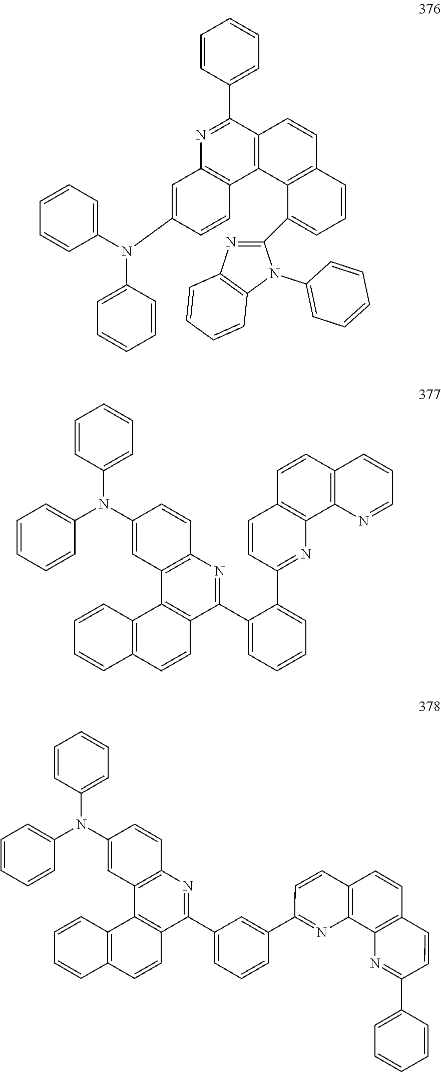 [SMILES: c1ccc(-c2nc3cc(N(c4ccccc4)c4ccccc4)ccc3c3c2ccc2cccc(-c4nc5ccccc5n4-c4ccccc4)c23)cc1 ; c1ccc(N(c2ccccc2)c2ccc3nc(-c4ccccc4-c4ccc5ccc6cccnc6c5n4)c4ccc5ccccc5c4c3c2)cc1 ; c1ccc(-c2ccc3ccc4ccc(-c5cccc(-c6nc7ccc(N(c8ccccc8)c8ccccc8)cc7c7c6ccc6ccccc67)c5)nc4c3n2)cc1]

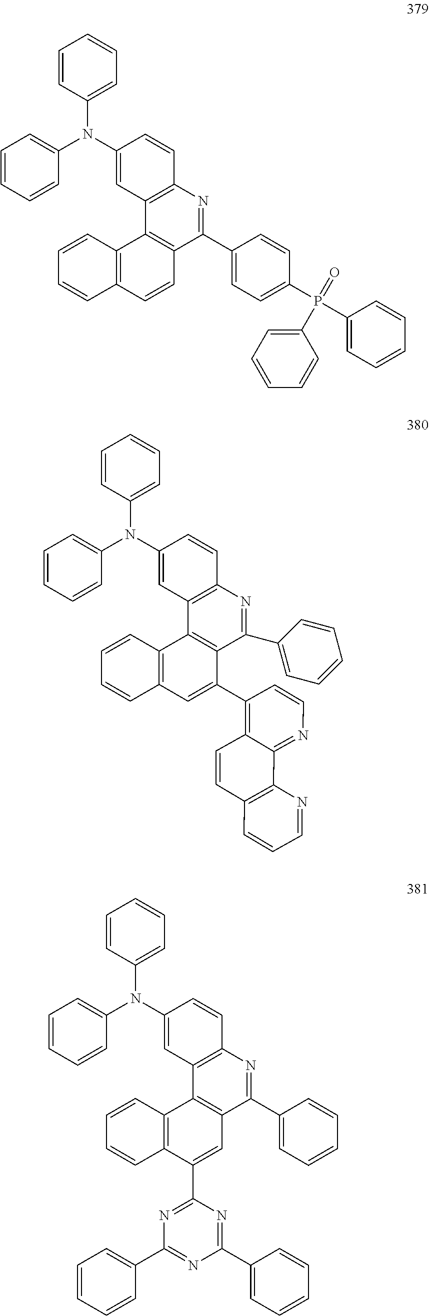 [SMILES: O=P(c1ccccc1)(c1ccccc1)c1ccc(-c2nc3ccc(N(c4ccccc4)c4ccccc4)cc3c3c2ccc2ccccc23)cc1 ; c1ccc(-c2nc3ccc(N(c4ccccc4)c4ccccc4)cc3c3c2c(-c2ccnc4c2ccc2cccnc24)cc2ccccc23)cc1 ; c1ccc(-c2nc(-c3ccccc3)nc(-c3cc4c(-c5ccccc5)nc5ccc(N(c6ccccc6)c6ccccc6)cc5c4c4ccccc34)n2)cc1]

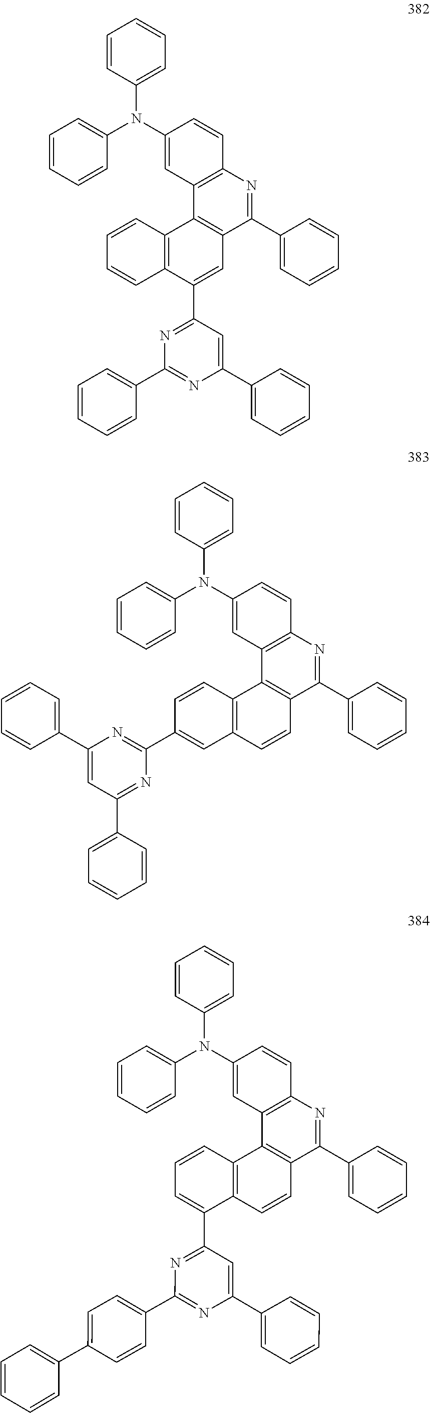 [SMILES: c1ccc(-c2cc(-c3cc4c(-c5ccccc5)nc5ccc(N(c6ccccc6)c6ccccc6)cc5c4c4ccccc34)nc(-c3ccccc3)n2)cc1 ; c1ccc(-c2cc(-c3ccccc3)nc(-c3ccc4c(ccc5c(-c6ccccc6)nc6ccc(N(c7ccccc7)c7ccccc7)cc6c54)c3)n2)cc1 ; c1ccc(-c2ccc(-c3nc(-c4ccccc4)cc(-c4cccc5c4ccc4c(-c6ccccc6)nc6ccc(N(c7ccccc7)c7ccccc7)cc6c45)n3)cc2)cc1]

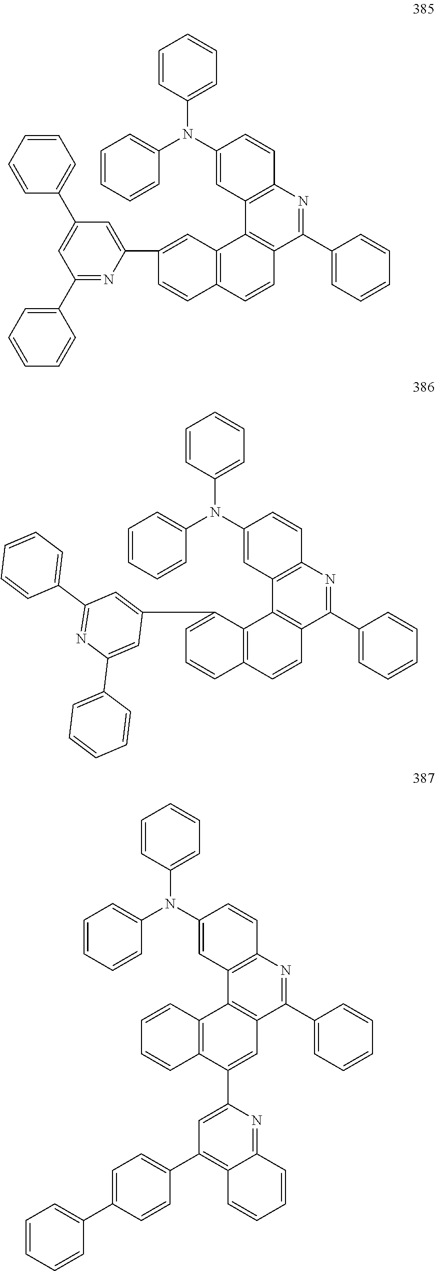 [SMILES: c1ccc(-c2cc(-c3ccccc3)nc(-c3ccc4ccc5c(-c6ccccc6)nc6ccc(N(c7ccccc7)c7ccccc7)cc6c5c4c3)c2)cc1 ; c1ccc(-c2cc(-c3cccc4ccc5c(-c6ccccc6)nc6ccc(N(c7ccccc7)c7ccccc7)cc6c5c34)cc(-c3ccccc3)n2)cc1 ; c1ccc(-c2ccc(-c3cc(-c4cc5c(-c6ccccc6)nc6ccc(N(c7ccccc7)c7ccccc7)cc6c5c5ccccc45)nc4ccccc34)cc2)cc1]

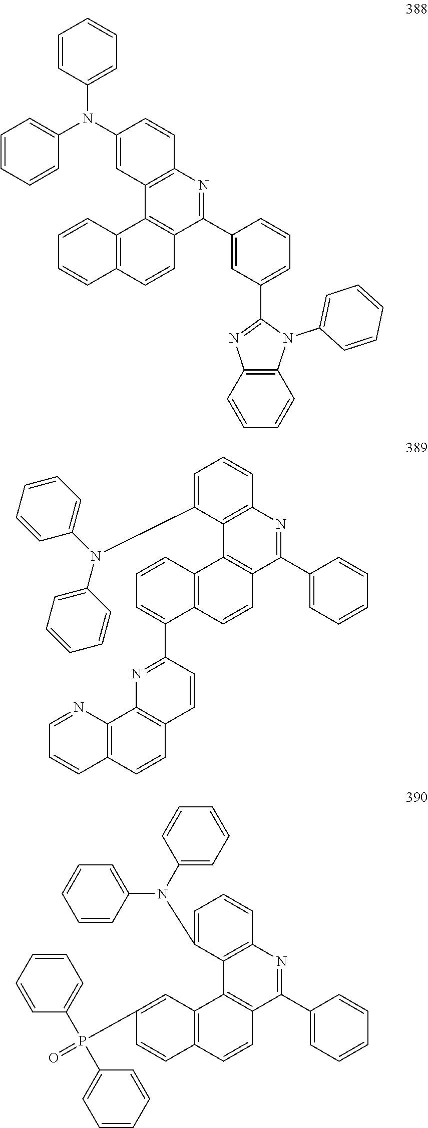

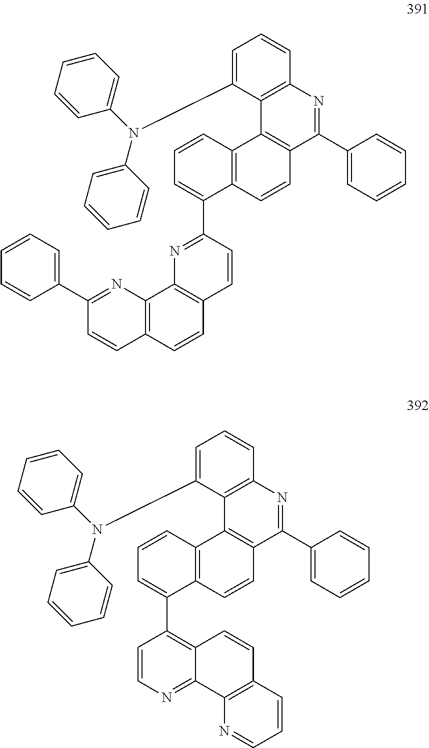 [SMILES: c1ccc(-c2ccc3ccc4ccc(-c5cccc6c5ccc5c(-c7ccccc7)nc7cccc(N(c8ccccc8)c8ccccc8)c7c56)nc4c3n2)cc1 ; c1ccc(-c2nc3cccc(N(c4ccccc4)c4ccccc4)c3c3c2ccc2c(-c4ccnc5c4ccc4cccnc45)cccc23)cc1]

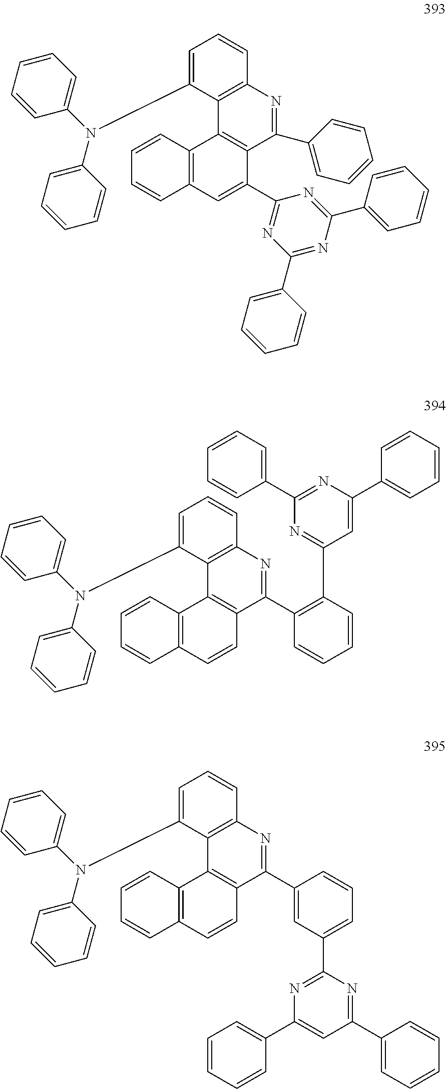 [SMILES: c1ccc(-c2nc(-c3ccccc3)nc(-c3cc4ccccc4c4c3c(-c3ccccc3)nc3cccc(N(c5ccccc5)c5ccccc5)c34)n2)cc1 ; c1ccc(-c2cc(-c3ccccc3-c3nc4cccc(N(c5ccccc5)c5ccccc5)c4c4c3ccc3ccccc34)nc(-c3ccccc3)n2)cc1 ; c1ccc(-c2cc(-c3ccccc3)nc(-c3cccc(-c4nc5cccc(N(c6ccccc6)c6ccccc6)c5c5c4ccc4ccccc45)c3)n2)cc1]

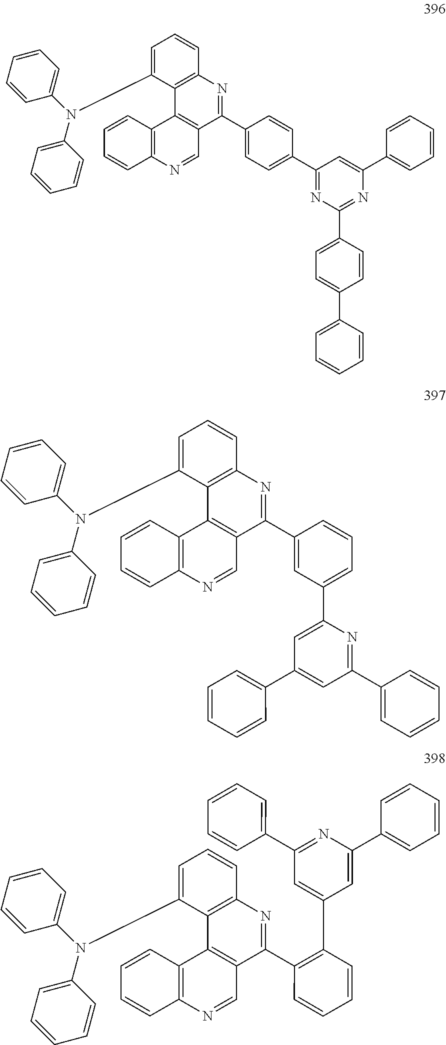

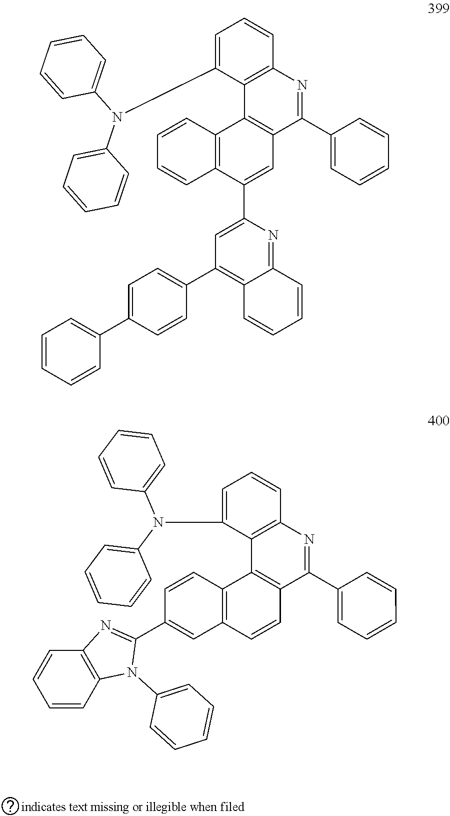 [SMILES: c1ccc(-c2ccc(-c3cc(-c4cc5c(-c6ccccc6)nc6cccc(N(c7ccccc7)c7ccccc7)c6c5c5ccccc45)nc4ccccc34)cc2)cc1 ; c1ccc(-c2nc3cccc(N(c4ccccc4)c4ccccc4)c3c3c2ccc2cc(-c4nc5ccccc5n4-c4ccccc4)ccc23)cc1]

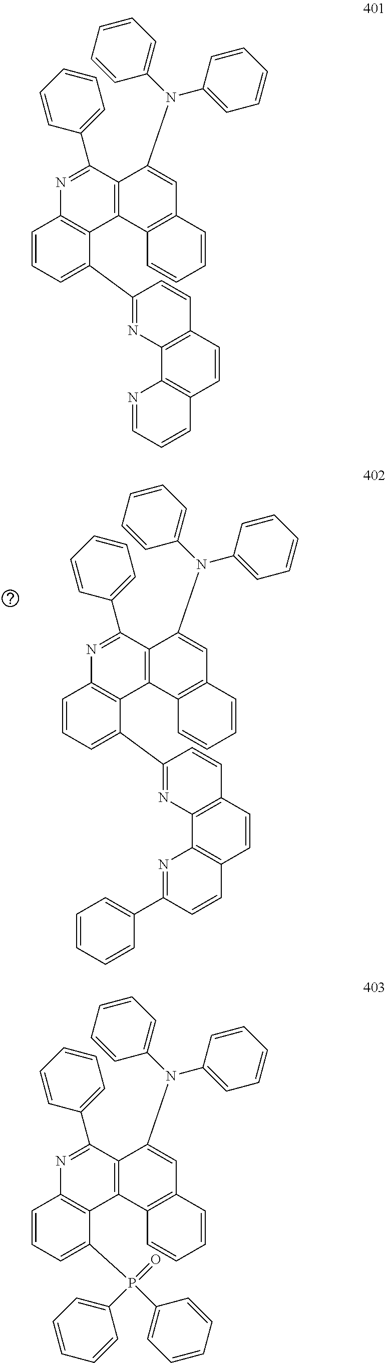

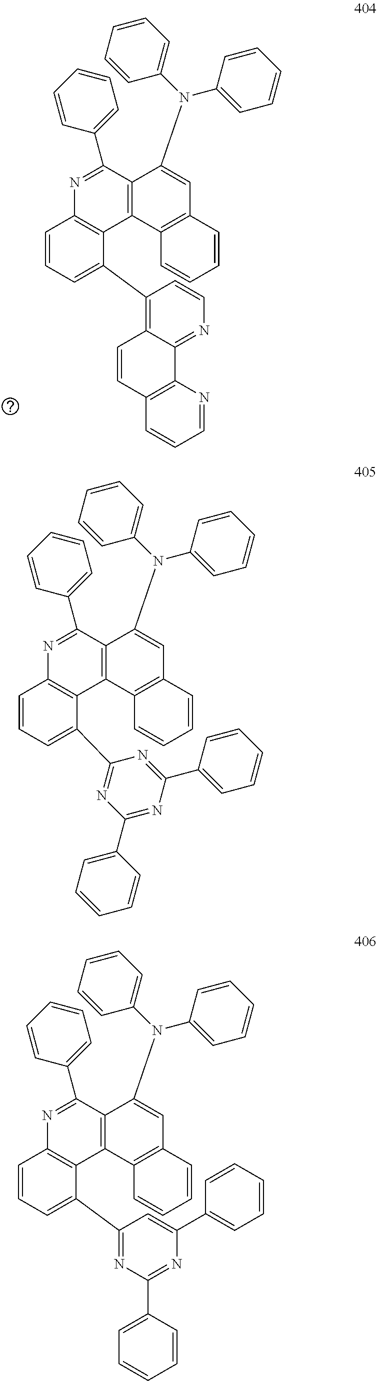

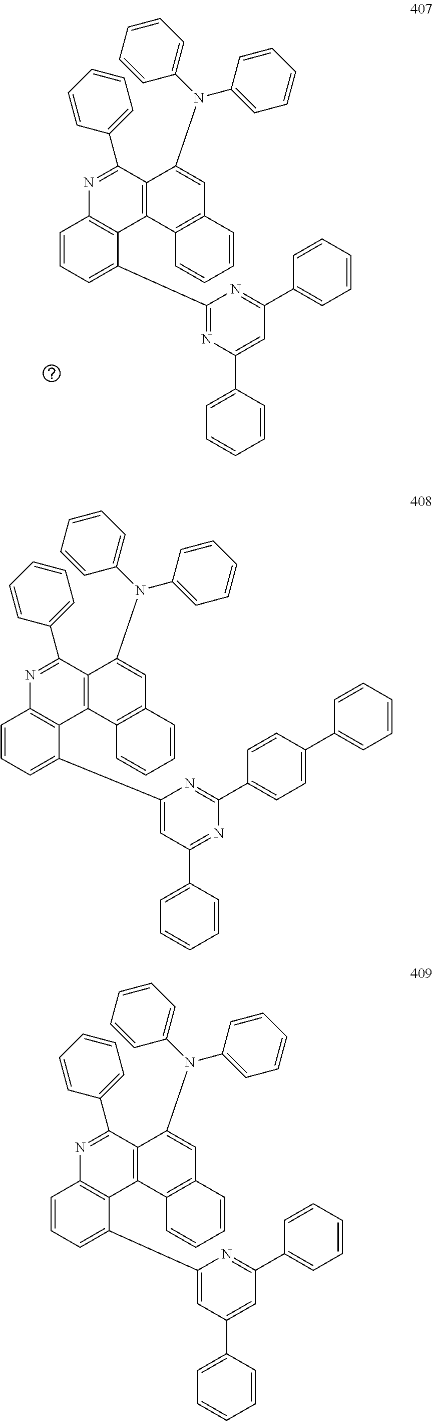 [SMILES: c1ccc(-c2cc(-c3ccccc3)nc(-c3cccc4nc(-c5ccccc5)c5c(N(c6ccccc6)c6ccccc6)cc6ccccc6c5c34)n2)cc1 ; c1ccc(-c2ccc(-c3nc(-c4ccccc4)cc(-c4cccc5nc(-c6ccccc6)c6c(N(c7ccccc7)c7ccccc7)cc7ccccc7c6c45)n3)cc2)cc1 ; c1ccc(-c2cc(-c3ccccc3)nc(-c3cccc4nc(-c5ccccc5)c5c(N(c6ccccc6)c6ccccc6)cc6ccccc6c5c34)c2)cc1]

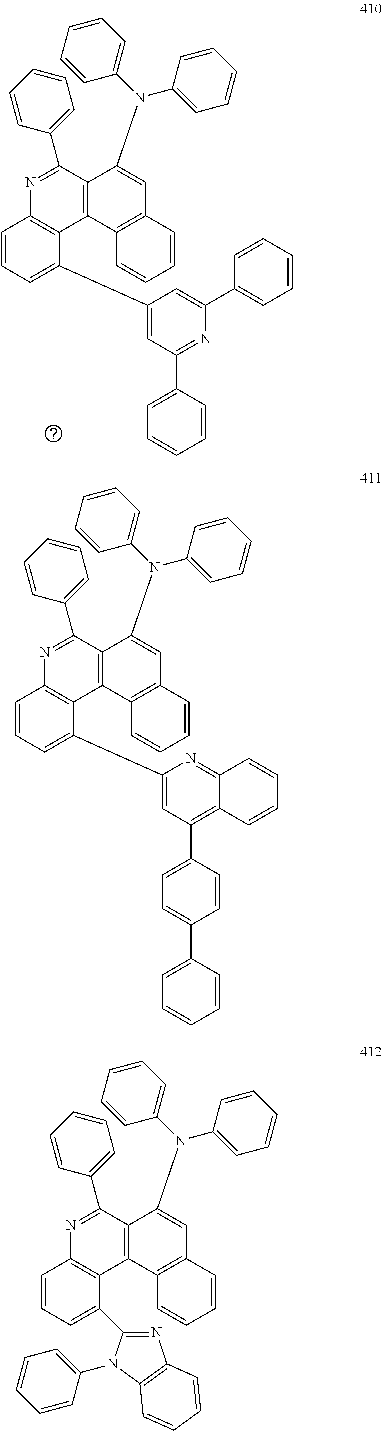 [SMILES: c1ccc(-c2cc(-c3cccc4nc(-c5ccccc5)c5c(N(c6ccccc6)c6ccccc6)cc6ccccc6c5c34)cc(-c3ccccc3)n2)cc1 ; c1ccc(-c2ccc(-c3cc(-c4cccc5nc(-c6ccccc6)c6c(N(c7ccccc7)c7ccccc7)cc7ccccc7c6c45)nc4ccccc34)cc2)cc1 ; c1ccc(-c2nc3cccc(-c4nc5ccccc5n4-c4ccccc4)c3c3c2c(N(c2ccccc2)c2ccccc2)cc2ccccc23)cc1]

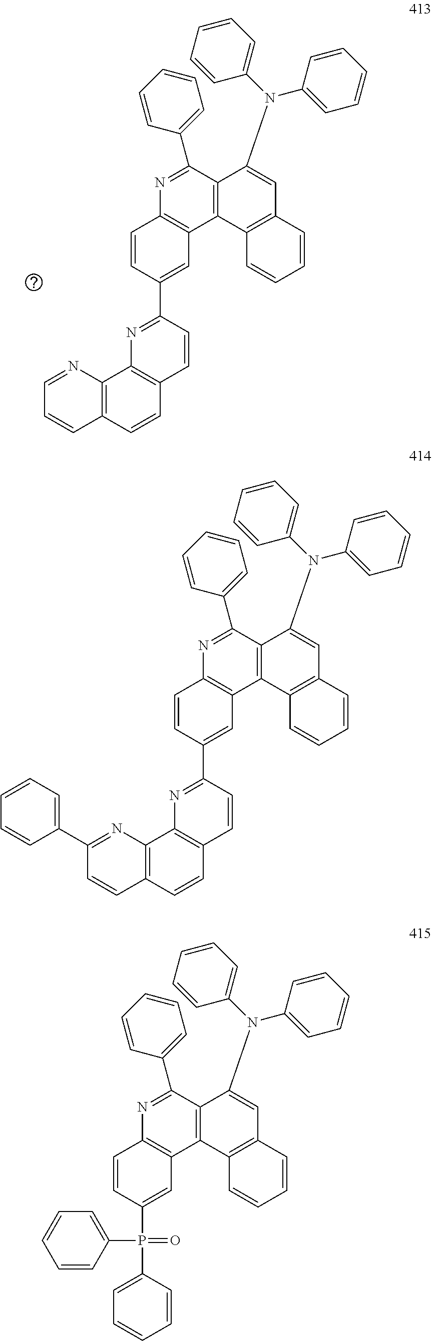 [SMILES: c1ccc(-c2nc3ccc(-c4ccc5ccc6cccnc6c5n4)cc3c3c2c(N(c2ccccc2)c2ccccc2)cc2ccccc23)cc1 ; c1ccc(-c2ccc3ccc4ccc(-c5ccc6nc(-c7ccccc7)c7c(N(c8ccccc8)c8ccccc8)cc8ccccc8c7c6c5)nc4c3n2)cc1 ; O=P(c1ccccc1)(c1ccccc1)c1ccc2nc(-c3ccccc3)c3c(N(c4ccccc4)c4ccccc4)cc4ccccc4c3c2c1]

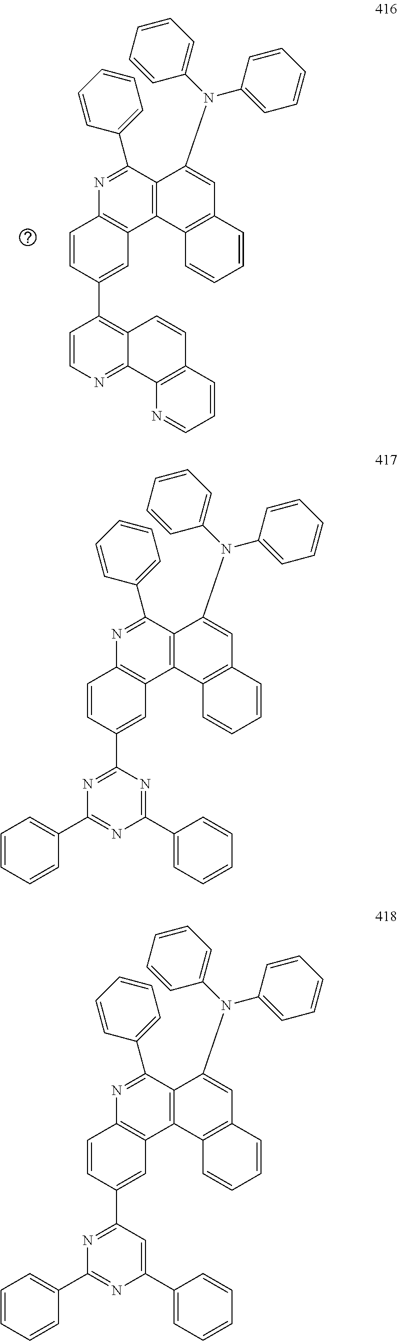

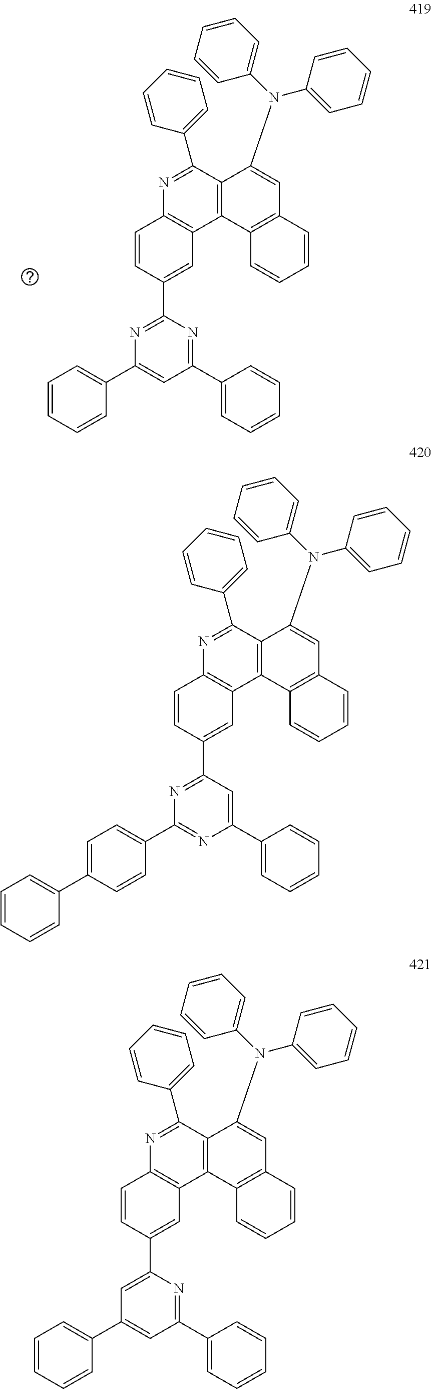 [SMILES: c1ccc(-c2cc(-c3ccccc3)nc(-c3ccc4nc(-c5ccccc5)c5c(N(c6ccccc6)c6ccccc6)cc6ccccc6c5c4c3)n2)cc1 ; c1ccc(-c2ccc(-c3nc(-c4ccccc4)cc(-c4ccc5nc(-c6ccccc6)c6c(N(c7ccccc7)c7ccccc7)cc7ccccc7c6c5c4)n3)cc2)cc1 ; c1ccc(-c2cc(-c3ccccc3)nc(-c3ccc4nc(-c5ccccc5)c5c(N(c6ccccc6)c6ccccc6)cc6ccccc6c5c4c3)c2)cc1]

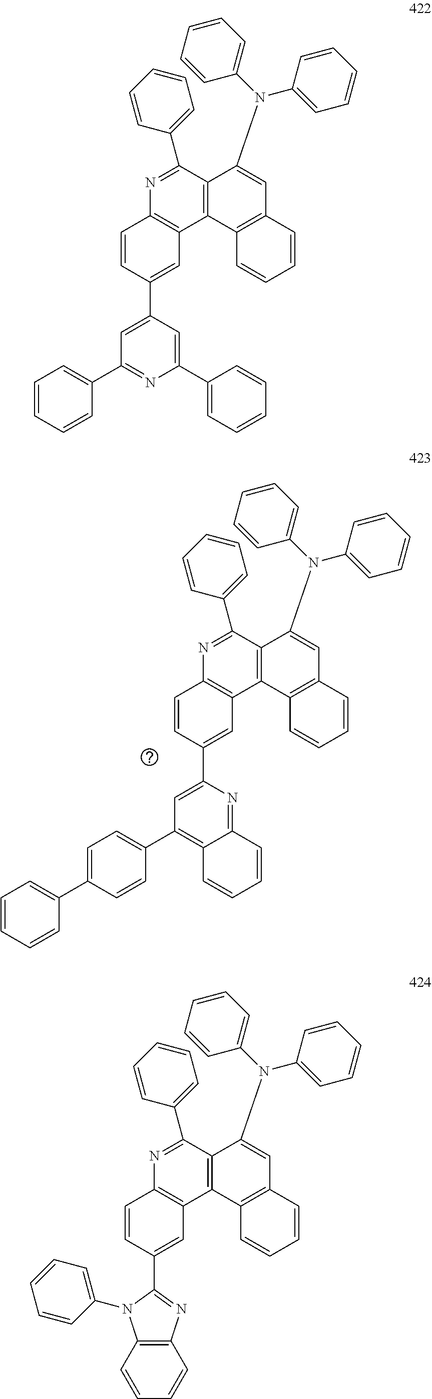 [SMILES: c1ccc(-c2cc(-c3ccc4nc(-c5ccccc5)c5c(N(c6ccccc6)c6ccccc6)cc6ccccc6c5c4c3)cc(-c3ccccc3)n2)cc1 ; c1ccc(-c2ccc(-c3cc(-c4ccc5nc(-c6ccccc6)c6c(N(c7ccccc7)c7ccccc7)cc7ccccc7c6c5c4)nc4ccccc34)cc2)cc1 ; c1ccc(-c2nc3ccc(-c4nc5ccccc5n4-c4ccccc4)cc3c3c2c(N(c2ccccc2)c2ccccc2)cc2ccccc23)cc1]

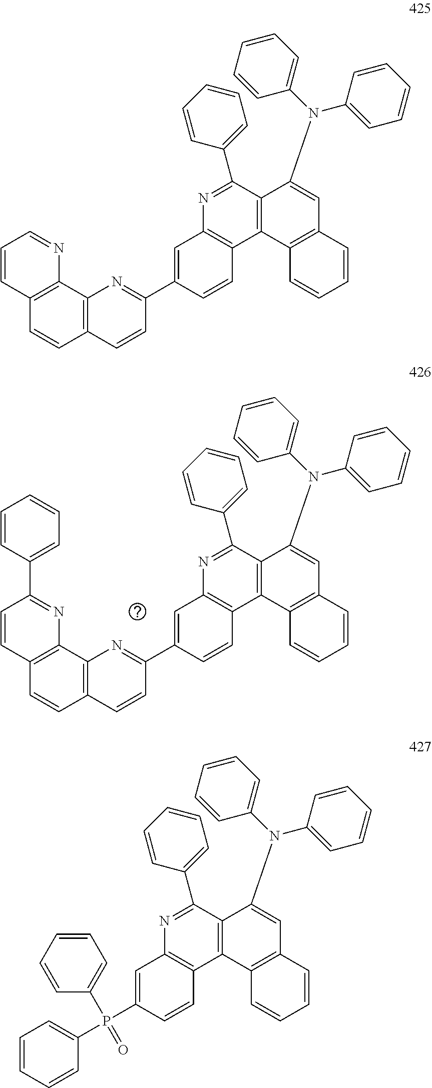 [SMILES: c1ccc(-c2nc3cc(-c4ccc5ccc6cccnc6c5n4)ccc3c3c2c(N(c2ccccc2)c2ccccc2)cc2ccccc23)cc1 ; c1ccc(-c2ccc3ccc4ccc(-c5ccc6c(c5)nc(-c5ccccc5)c5c(N(c7ccccc7)c7ccccc7)cc7ccccc7c56)nc4c3n2)cc1 ; O=P(c1ccccc1)(c1ccccc1)c1ccc2c(c1)nc(-c1ccccc1)c1c(N(c3ccccc3)c3ccccc3)cc3ccccc3c12]

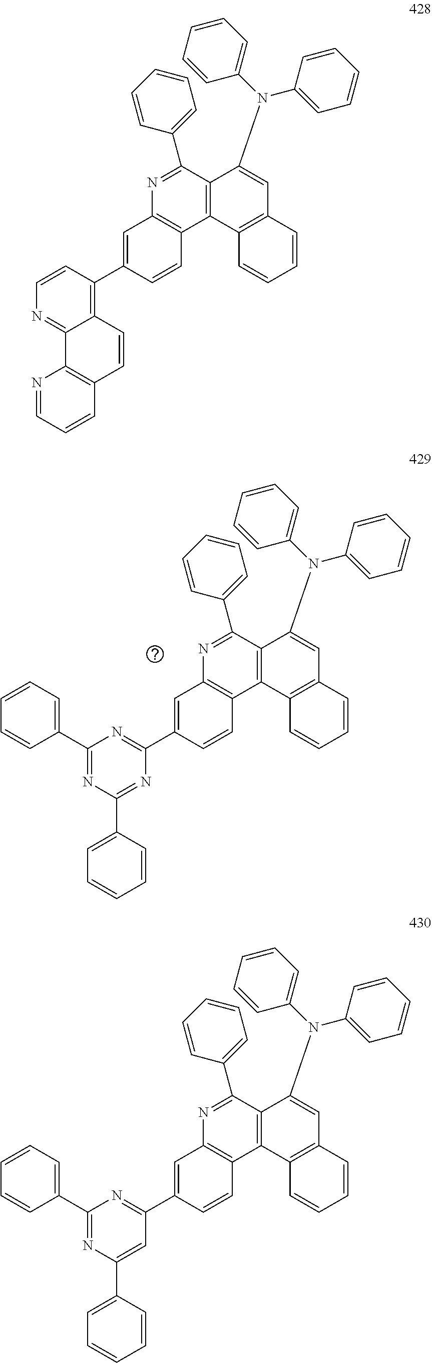 [SMILES: c1ccc(-c2nc3cc(-c4ccnc5c4ccc4cccnc45)ccc3c3c2c(N(c2ccccc2)c2ccccc2)cc2ccccc23)cc1 ; c1ccc(-c2nc(-c3ccccc3)nc(-c3ccc4c(c3)nc(-c3ccccc3)c3c(N(c5ccccc5)c5ccccc5)cc5ccccc5c34)n2)cc1 ; c1ccc(-c2cc(-c3ccc4c(c3)nc(-c3ccccc3)c3c(N(c5ccccc5)c5ccccc5)cc5ccccc5c34)nc(-c3ccccc3)n2)cc1]

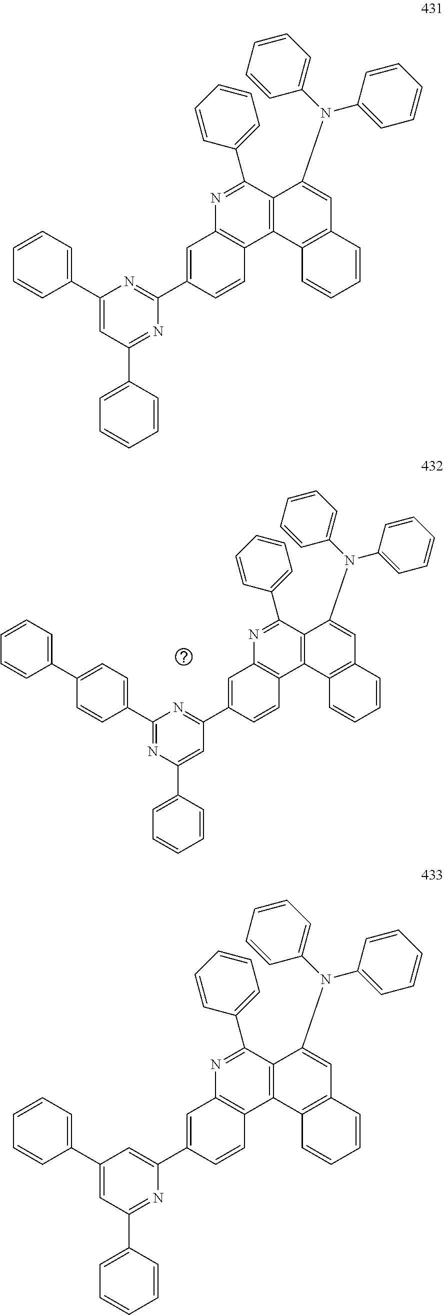 [SMILES: c1ccc(-c2cc(-c3ccccc3)nc(-c3ccc4c(c3)nc(-c3ccccc3)c3c(N(c5ccccc5)c5ccccc5)cc5ccccc5c34)n2)cc1 ; c1ccc(-c2ccc(-c3nc(-c4ccccc4)cc(-c4ccc5c(c4)nc(-c4ccccc4)c4c(N(c6ccccc6)c6ccccc6)cc6ccccc6c45)n3)cc2)cc1 ; c1ccc(-c2cc(-c3ccccc3)nc(-c3ccc4c(c3)nc(-c3ccccc3)c3c(N(c5ccccc5)c5ccccc5)cc5ccccc5c34)c2)cc1]

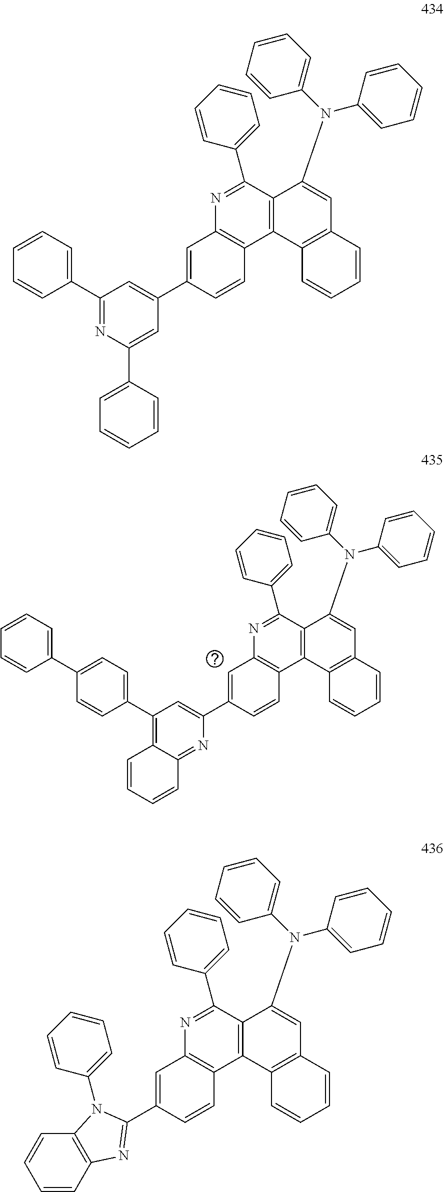 [SMILES: c1ccc(-c2cc(-c3ccc4c(c3)nc(-c3ccccc3)c3c(N(c5ccccc5)c5ccccc5)cc5ccccc5c34)cc(-c3ccccc3)n2)cc1 ; c1ccc(-c2ccc(-c3cc(-c4ccc5c(c4)nc(-c4ccccc4)c4c(N(c6ccccc6)c6ccccc6)cc6ccccc6c45)nc4ccccc34)cc2)cc1 ; c1ccc(-c2nc3cc(-c4nc5ccccc5n4-c4ccccc4)ccc3c3c2c(N(c2ccccc2)c2ccccc2)cc2ccccc23)cc1]

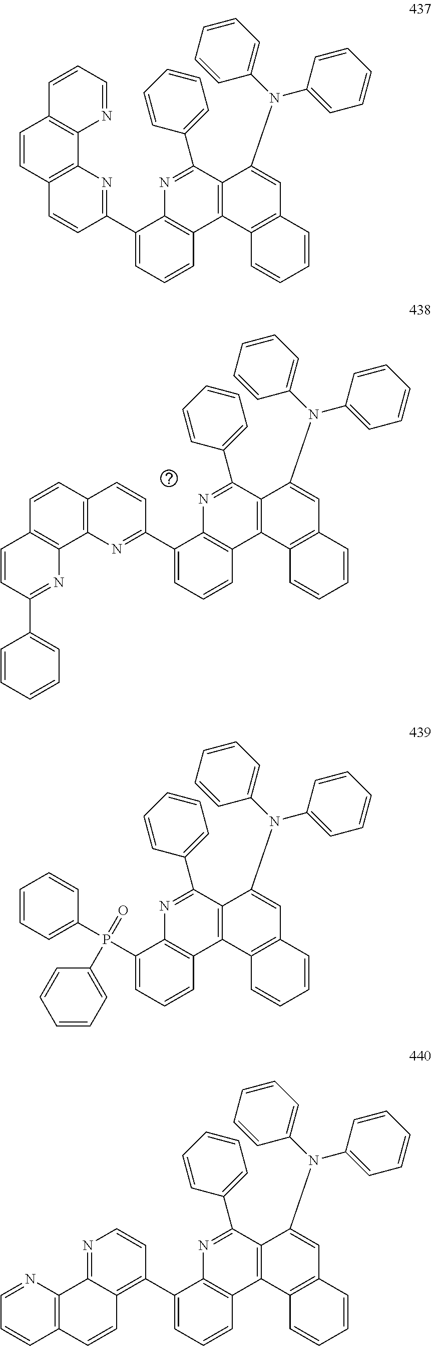 [SMILES: c1ccc(-c2nc3c(-c4ccc5ccc6cccnc6c5n4)cccc3c3c2c(N(c2ccccc2)c2ccccc2)cc2ccccc23)cc1 ; c1ccc(-c2ccc3ccc4ccc(-c5cccc6c5nc(-c5ccccc5)c5c(N(c7ccccc7)c7ccccc7)cc7ccccc7c56)nc4c3n2)cc1 ; O=P(c1ccccc1)(c1ccccc1)c1cccc2c1nc(-c1ccccc1)c1c(N(c3ccccc3)c3ccccc3)cc3ccccc3c12 ; c1ccc(-c2nc3c(-c4ccnc5c4ccc4cccnc45)cccc3c3c2c(N(c2ccccc2)c2ccccc2)cc2ccccc23)cc1]

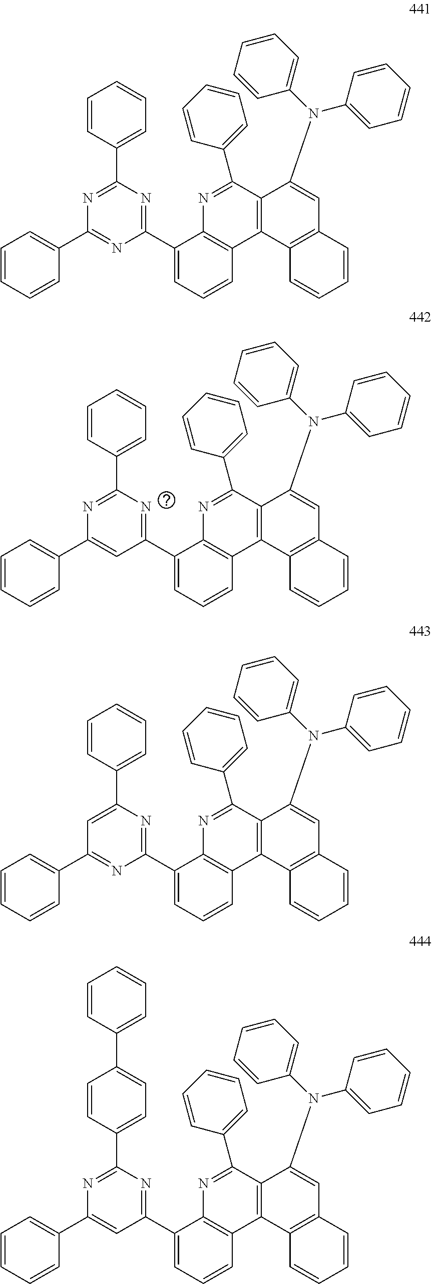 [SMILES: c1ccc(-c2nc(-c3ccccc3)nc(-c3cccc4c3nc(-c3ccccc3)c3c(N(c5ccccc5)c5ccccc5)cc5ccccc5c34)n2)cc1 ; c1ccc(-c2cc(-c3cccc4c3nc(-c3ccccc3)c3c(N(c5ccccc5)c5ccccc5)cc5ccccc5c34)nc(-c3ccccc3)n2)cc1 ; c1ccc(-c2cc(-c3ccccc3)nc(-c3cccc4c3nc(-c3ccccc3)c3c(N(c5ccccc5)c5ccccc5)cc5ccccc5c34)n2)cc1 ; c1ccc(-c2ccc(-c3nc(-c4ccccc4)cc(-c4cccc5c4nc(-c4ccccc4)c4c(N(c6ccccc6)c6ccccc6)cc6ccccc6c45)n3)cc2)cc1]

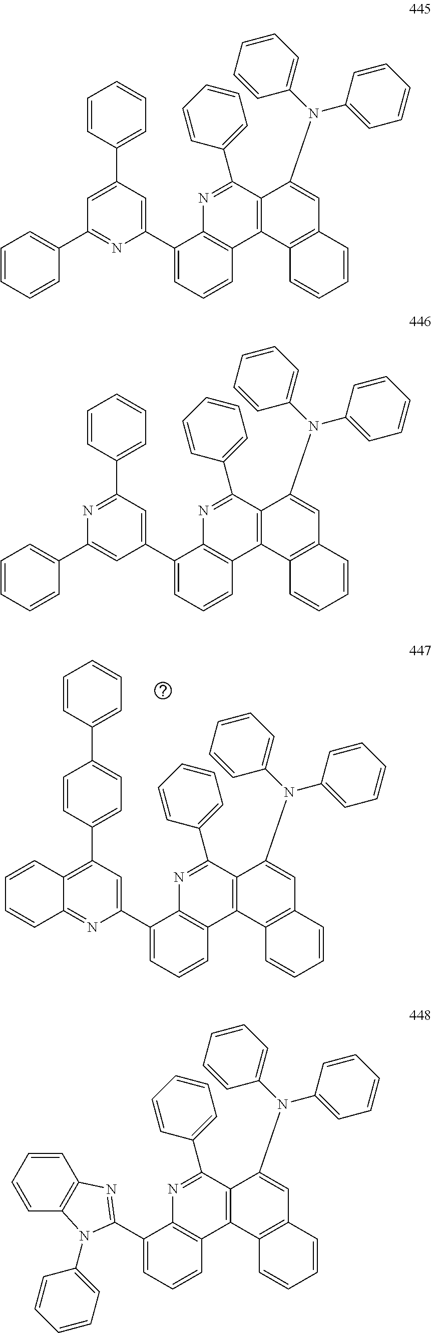 [SMILES: c1ccc(-c2cc(-c3ccccc3)nc(-c3cccc4c3nc(-c3ccccc3)c3c(N(c5ccccc5)c5ccccc5)cc5ccccc5c34)c2)cc1 ; c1ccc(-c2cc(-c3cccc4c3nc(-c3ccccc3)c3c(N(c5ccccc5)c5ccccc5)cc5ccccc5c34)cc(-c3ccccc3)n2)cc1 ; c1ccc(-c2ccc(-c3cc(-c4cccc5c4nc(-c4ccccc4)c4c(N(c6ccccc6)c6ccccc6)cc6ccccc6c45)nc4ccccc34)cc2)cc1 ; c1ccc(-c2nc3c(-c4nc5ccccc5n4-c4ccccc4)cccc3c3c2c(N(c2ccccc2)c2ccccc2)cc2ccccc23)cc1]

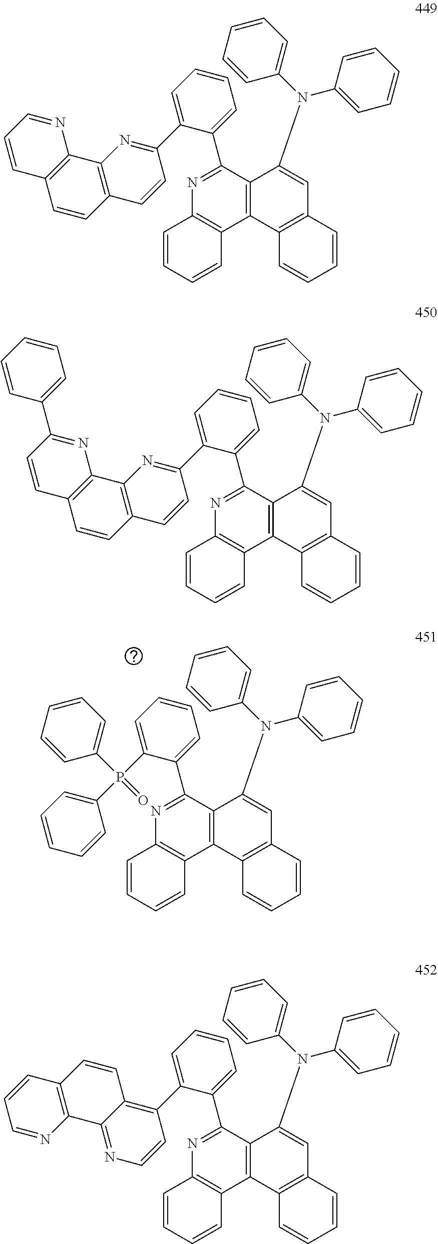

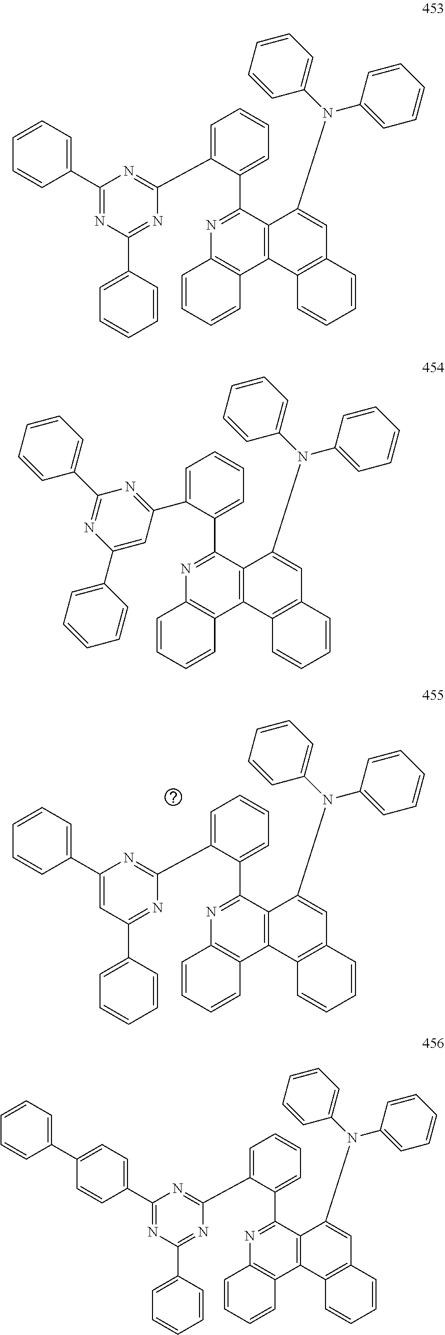 [SMILES: c1ccc(-c2nc(-c3ccccc3)nc(-c3ccccc3-c3nc4ccccc4c4c3c(N(c3ccccc3)c3ccccc3)cc3ccccc34)n2)cc1 ; c1ccc(-c2cc(-c3ccccc3-c3nc4ccccc4c4c3c(N(c3ccccc3)c3ccccc3)cc3ccccc34)nc(-c3ccccc3)n2)cc1 ; c1ccc(-c2cc(-c3ccccc3)nc(-c3ccccc3-c3nc4ccccc4c4c3c(N(c3ccccc3)c3ccccc3)cc3ccccc34)n2)cc1 ; c1ccc(-c2ccc(-c3nc(-c4ccccc4)nc(-c4ccccc4-c4nc5ccccc5c5c4c(N(c4ccccc4)c4ccccc4)cc4ccccc45)n3)cc2)cc1]

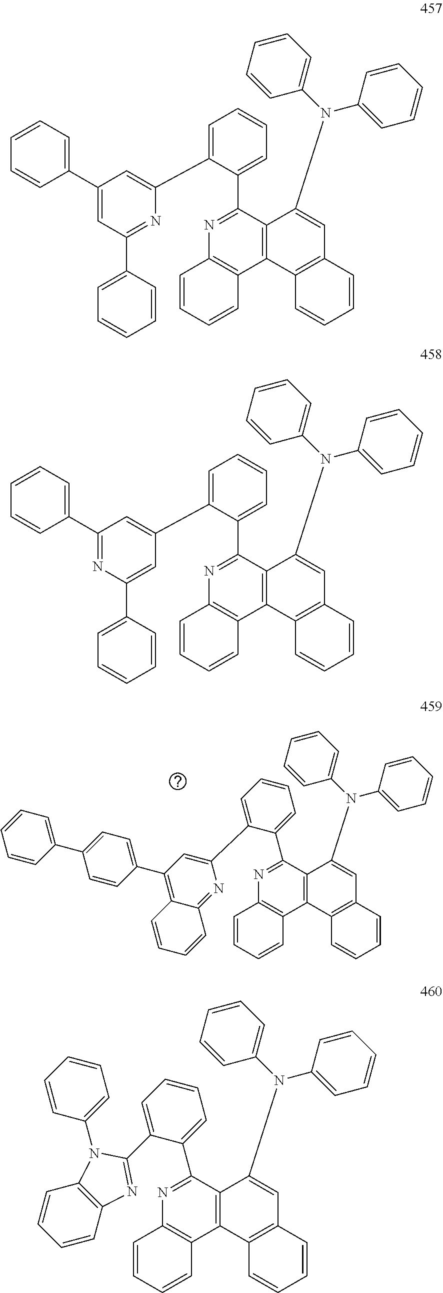

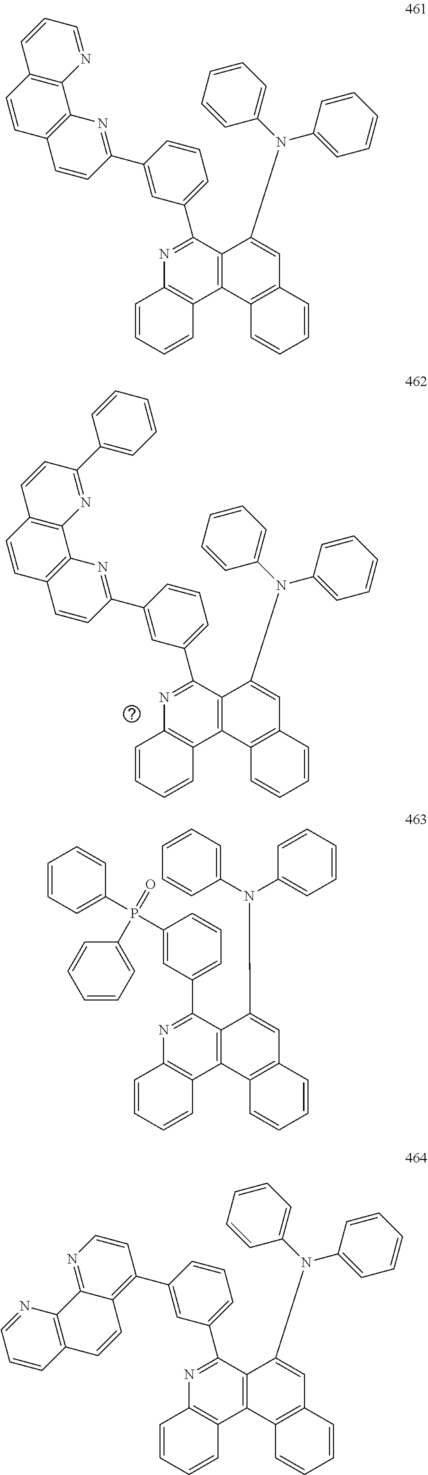

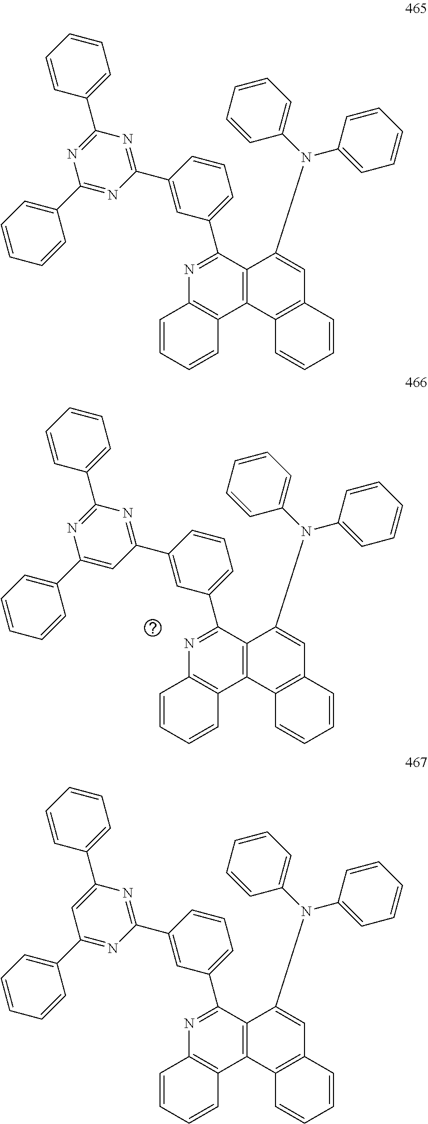 [SMILES: c1ccc(-c2nc(-c3ccccc3)nc(-c3cccc(-c4nc5ccccc5c5c4c(N(c4ccccc4)c4ccccc4)cc4ccccc45)c3)n2)cc1 ; c1ccc(-c2cc(-c3cccc(-c4nc5ccccc5c5c4c(N(c4ccccc4)c4ccccc4)cc4ccccc45)c3)nc(-c3ccccc3)n2)cc1 ; c1ccc(-c2cc(-c3ccccc3)nc(-c3cccc(-c4nc5ccccc5c5c4c(N(c4ccccc4)c4ccccc4)cc4ccccc45)c3)n2)cc1]

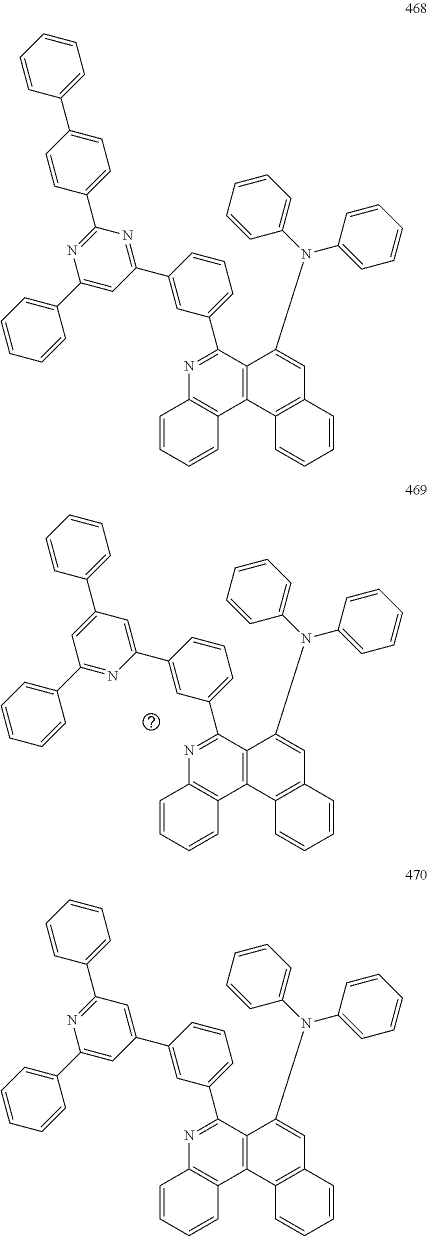 [SMILES: c1ccc(-c2ccc(-c3nc(-c4ccccc4)cc(-c4cccc(-c5nc6ccccc6c6c5c(N(c5ccccc5)c5ccccc5)cc5ccccc56)c4)n3)cc2)cc1 ; c1ccc(-c2cc(-c3ccccc3)nc(-c3cccc(-c4nc5ccccc5c5c4c(N(c4ccccc4)c4ccccc4)cc4ccccc45)c3)c2)cc1 ; c1ccc(-c2cc(-c3cccc(-c4nc5ccccc5c5c4c(N(c4ccccc4)c4ccccc4)cc4ccccc45)c3)cc(-c3ccccc3)n2)cc1]

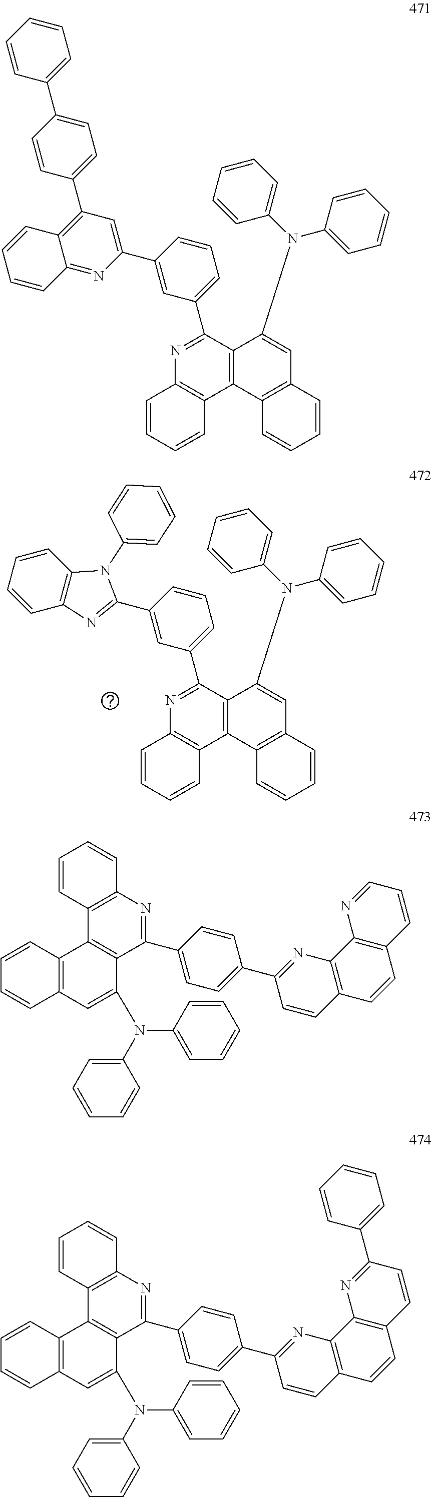

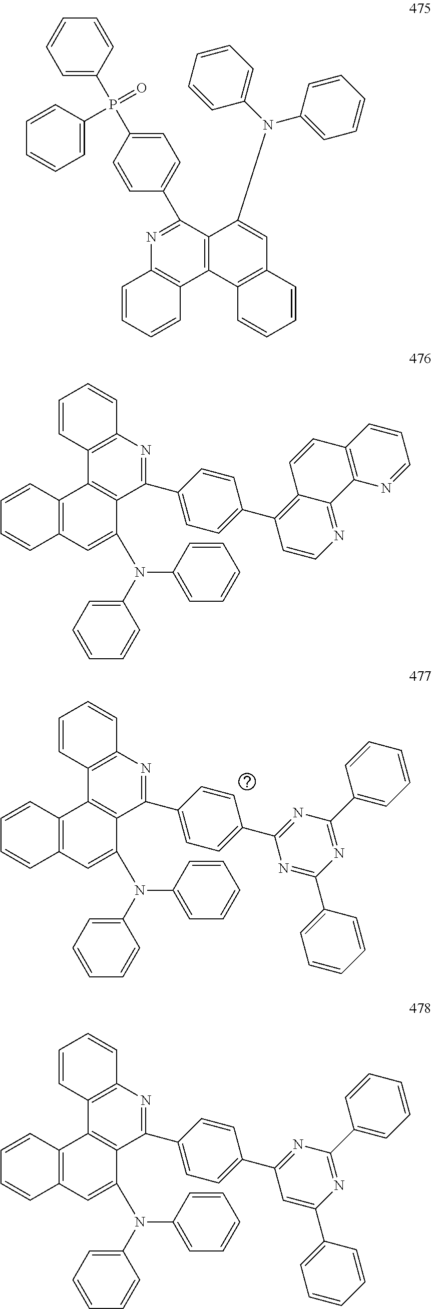

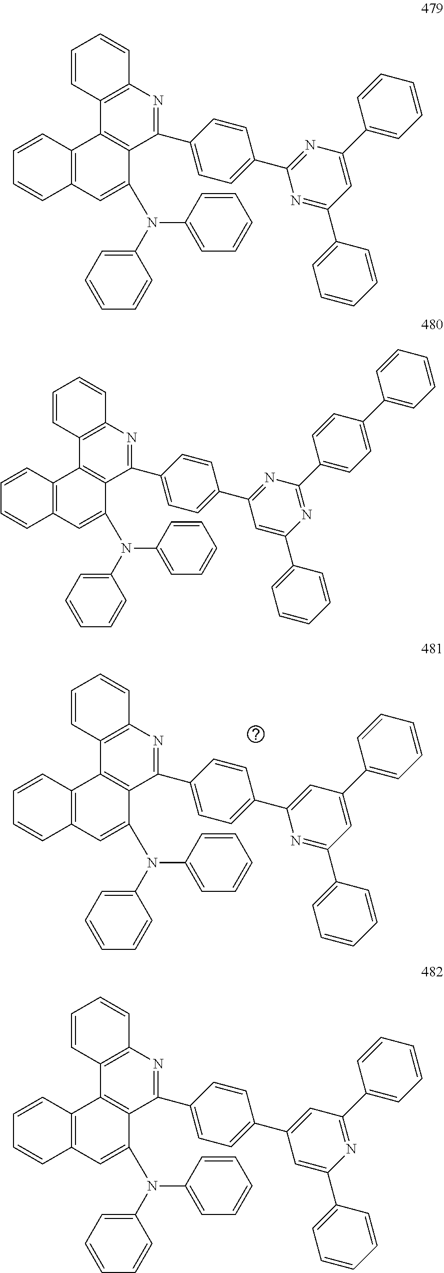 [SMILES: c1ccc(-c2cc(-c3ccccc3)nc(-c3ccc(-c4nc5ccccc5c5c4c(N(c4ccccc4)c4ccccc4)cc4ccccc45)cc3)n2)cc1 ; c1ccc(-c2ccc(-c3nc(-c4ccccc4)cc(-c4ccc(-c5nc6ccccc6c6c5c(N(c5ccccc5)c5ccccc5)cc5ccccc56)cc4)n3)cc2)cc1 ; c1ccc(-c2cc(-c3ccccc3)nc(-c3ccc(-c4nc5ccccc5c5c4c(N(c4ccccc4)c4ccccc4)cc4ccccc45)cc3)c2)cc1 ; c1ccc(-c2cc(-c3ccc(-c4nc5ccccc5c5c4c(N(c4ccccc4)c4ccccc4)cc4ccccc45)cc3)cc(-c3ccccc3)n2)cc1]

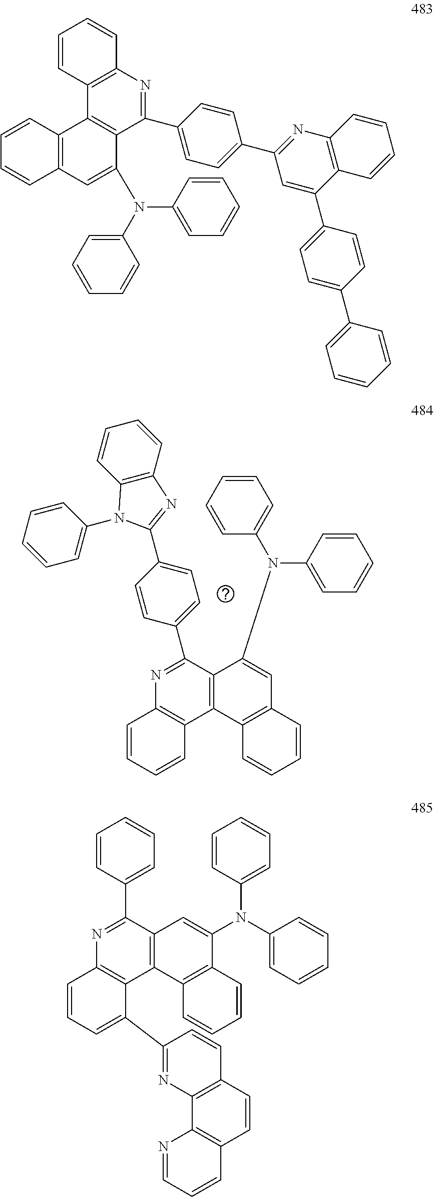 [SMILES: c1ccc(-c2ccc(-c3cc(-c4ccc(-c5nc6ccccc6c6c5c(N(c5ccccc5)c5ccccc5)cc5ccccc56)cc4)nc4ccccc34)cc2)cc1 ; c1ccc(N(c2ccccc2)c2cc3ccccc3c3c2c(-c2ccc(-c4nc5ccccc5n4-c4ccccc4)cc2)nc2ccccc23)cc1 ; c1ccc(-c2nc3cccc(-c4ccc5ccc6cccnc6c5n4)c3c3c2cc(N(c2ccccc2)c2ccccc2)c2ccccc23)cc1]

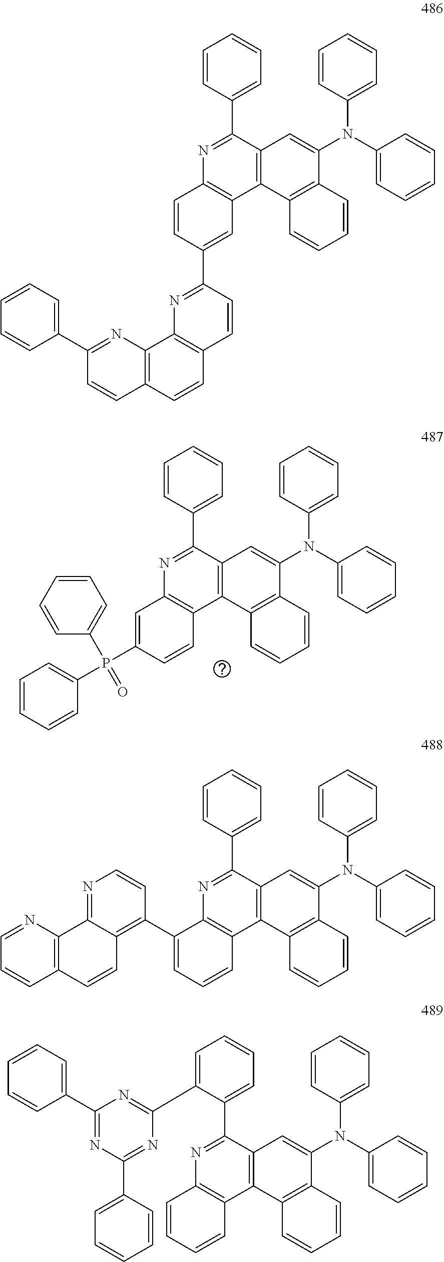 [SMILES: c1ccc(-c2ccc3ccc4ccc(-c5ccc6nc(-c7ccccc7)c7cc(N(c8ccccc8)c8ccccc8)c8ccccc8c7c6c5)nc4c3n2)cc1 ; O=P(c1ccccc1)(c1ccccc1)c1ccc2c(c1)nc(-c1ccccc1)c1cc(N(c3ccccc3)c3ccccc3)c3ccccc3c12 ; c1ccc(-c2nc3c(-c4ccnc5c4ccc4cccnc45)cccc3c3c2cc(N(c2ccccc2)c2ccccc2)c2ccccc23)cc1 ; c1ccc(-c2nc(-c3ccccc3)nc(-c3ccccc3-c3nc4ccccc4c4c3cc(N(c3ccccc3)c3ccccc3)c3ccccc34)n2)cc1]

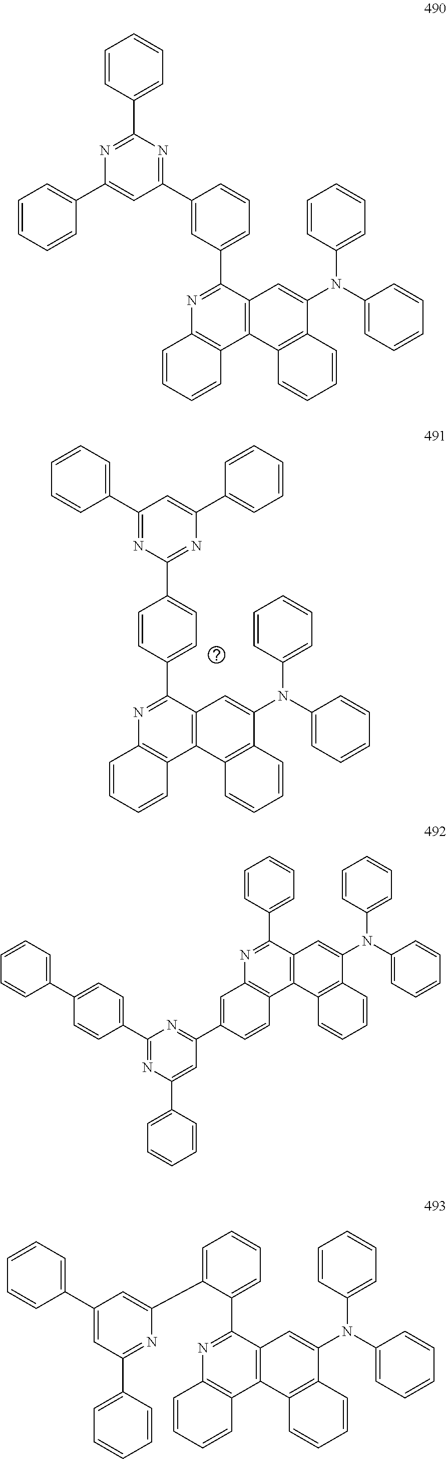

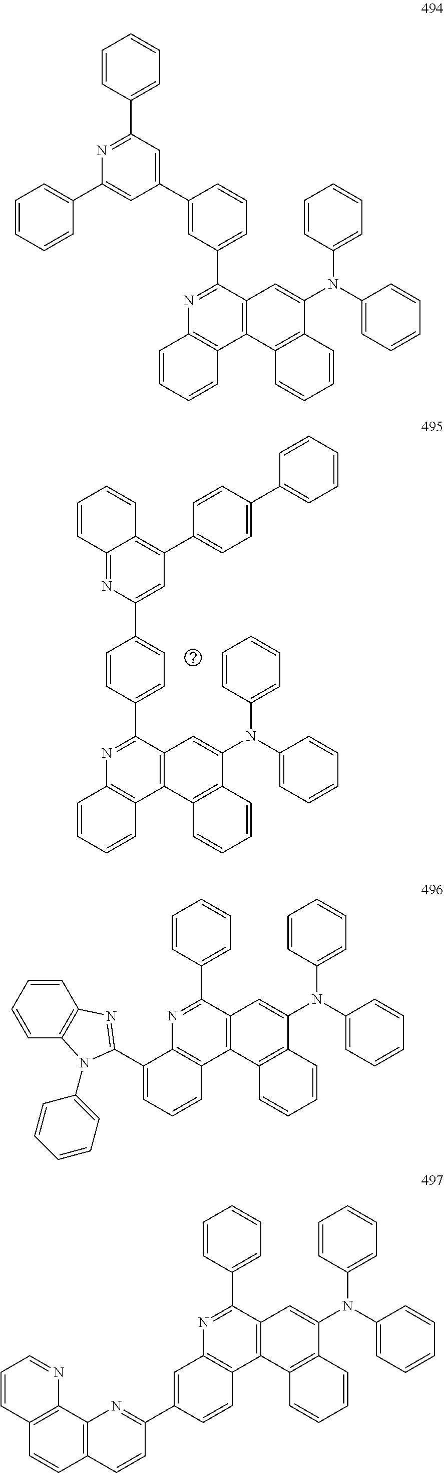

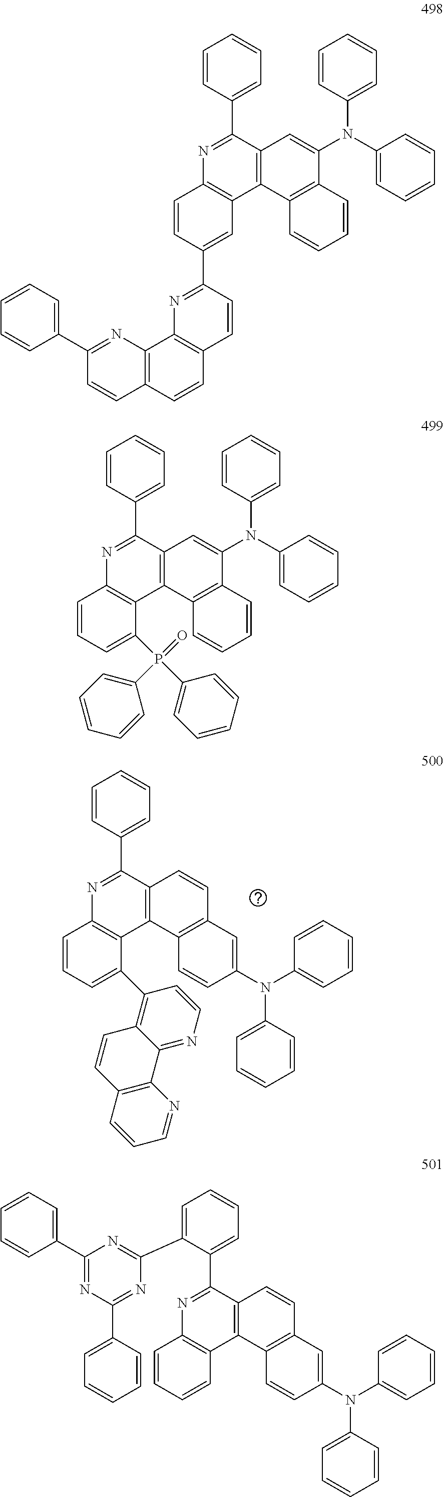

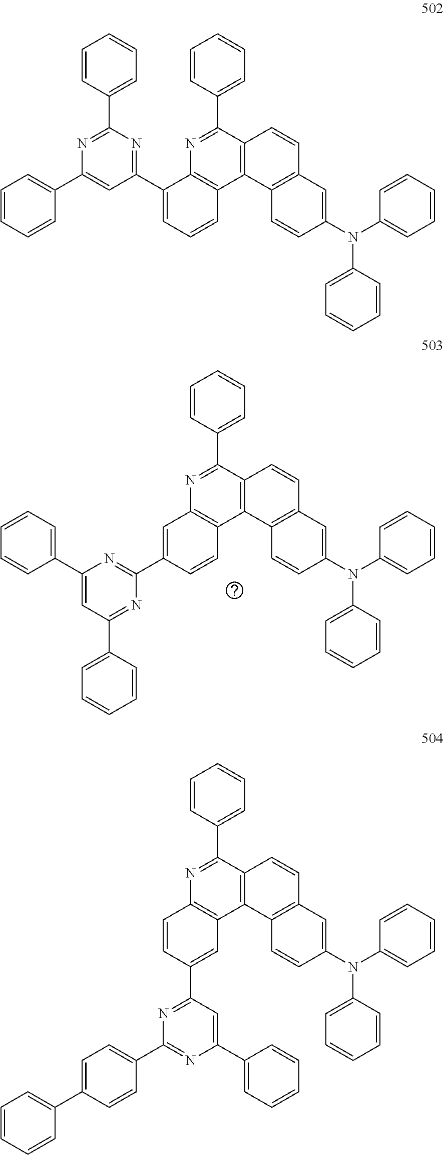 [SMILES: c1ccc(-c2cc(-c3cccc4c3nc(-c3ccccc3)c3ccc5cc(N(c6ccccc6)c6ccccc6)ccc5c34)nc(-c3ccccc3)n2)cc1 ; c1ccc(-c2cc(-c3ccccc3)nc(-c3ccc4c(c3)nc(-c3ccccc3)c3ccc5cc(N(c6ccccc6)c6ccccc6)ccc5c34)n2)cc1 ; c1ccc(-c2ccc(-c3nc(-c4ccccc4)cc(-c4ccc5nc(-c6ccccc6)c6ccc7cc(N(c8ccccc8)c8ccccc8)ccc7c6c5c4)n3)cc2)cc1]

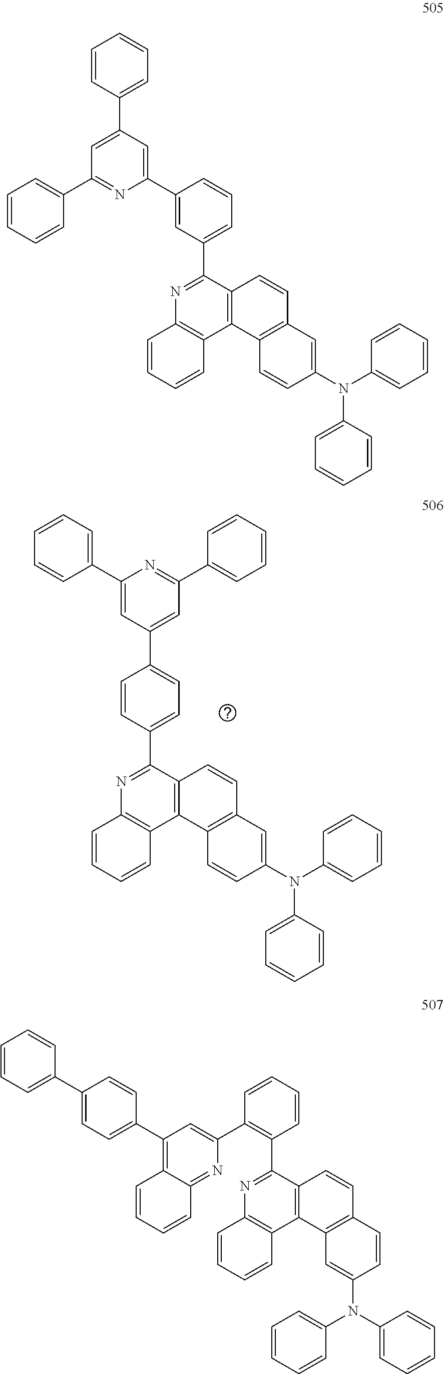 [SMILES: c1ccc(-c2cc(-c3ccccc3)nc(-c3cccc(-c4nc5ccccc5c5c4ccc4cc(N(c6ccccc6)c6ccccc6)ccc45)c3)c2)cc1 ; c1ccc(-c2cc(-c3ccc(-c4nc5ccccc5c5c4ccc4cc(N(c6ccccc6)c6ccccc6)ccc45)cc3)cc(-c3ccccc3)n2)cc1 ; c1ccc(-c2ccc(-c3cc(-c4ccccc4-c4nc5ccccc5c5c4ccc4ccc(N(c6ccccc6)c6ccccc6)cc45)nc4ccccc34)cc2)cc1]

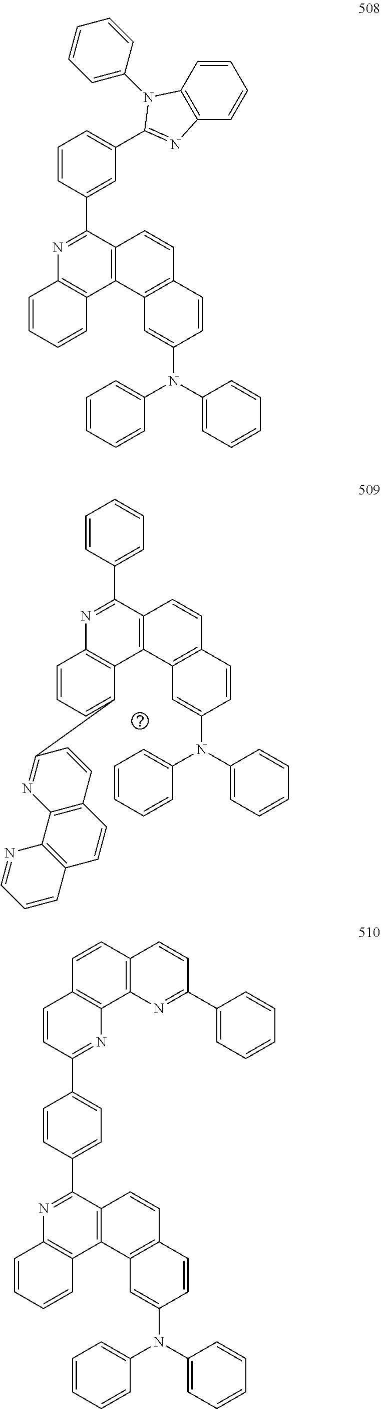 [SMILES: c1ccc(N(c2ccccc2)c2ccc3ccc4c(-c5cccc(-c6nc7ccccc7n6-c6ccccc6)c5)nc5ccccc5c4c3c2)cc1 ; c1ccc(-c2nc3cccc(-c4ccc5ccc6cccnc6c5n4)c3c3c2ccc2ccc(N(c4ccccc4)c4ccccc4)cc23)cc1 ; c1ccc(-c2ccc3ccc4ccc(-c5ccc(-c6nc7ccccc7c7c6ccc6ccc(N(c8ccccc8)c8ccccc8)cc67)cc5)nc4c3n2)cc1]

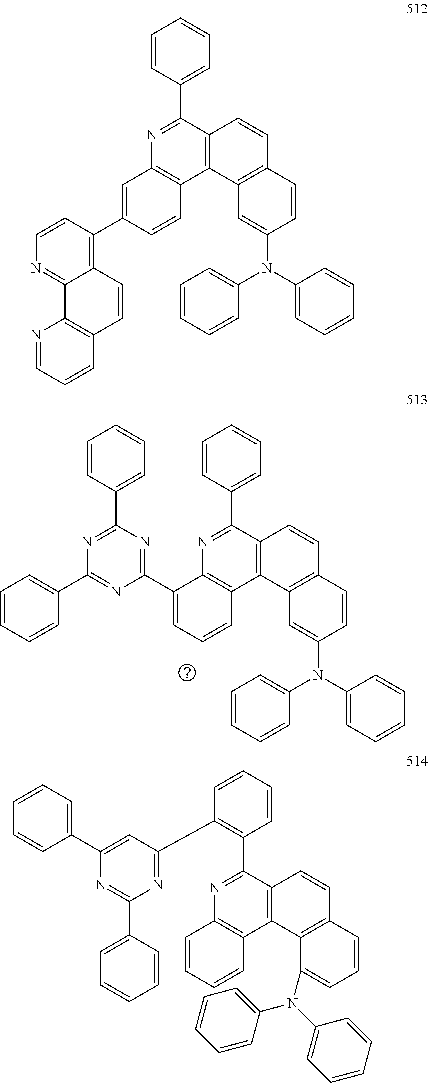

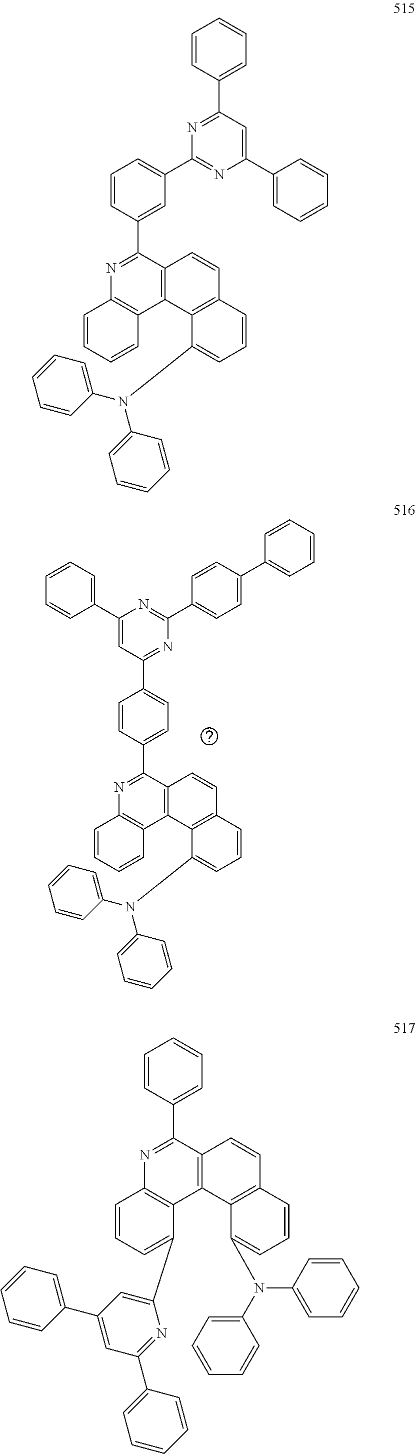 [SMILES: c1ccc(-c2cc(-c3ccccc3)nc(-c3cccc(-c4nc5ccccc5c5c4ccc4cccc(N(c6ccccc6)c6ccccc6)c45)c3)n2)cc1 ; c1ccc(-c2ccc(-c3nc(-c4ccccc4)cc(-c4ccc(-c5nc6ccccc6c6c5ccc5cccc(N(c7ccccc7)c7ccccc7)c56)cc4)n3)cc2)cc1 ; c1ccc(-c2cc(-c3ccccc3)nc(-c3cccc4nc(-c5ccccc5)c5ccc6cccc(N(c7ccccc7)c7ccccc7)c6c5c34)c2)cc1]

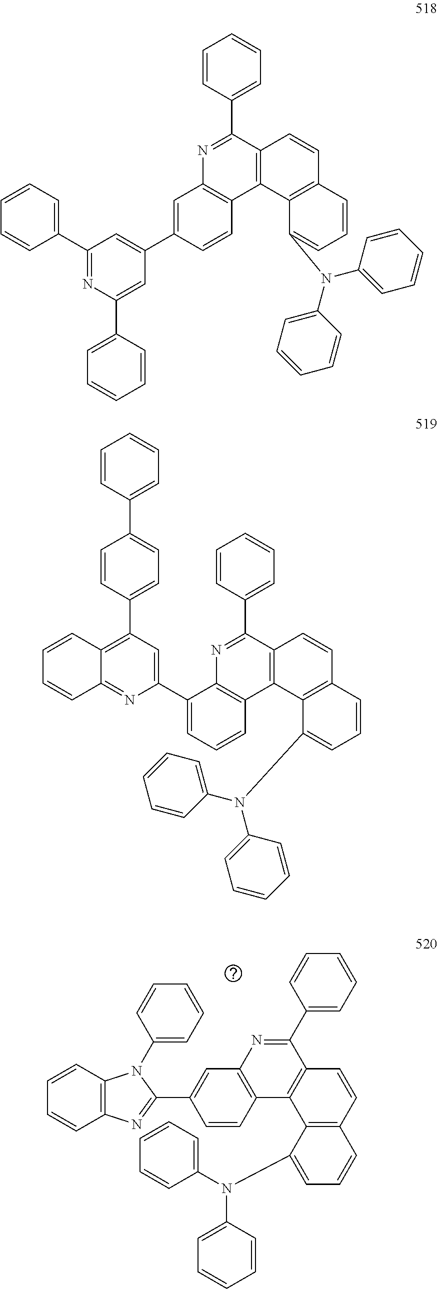

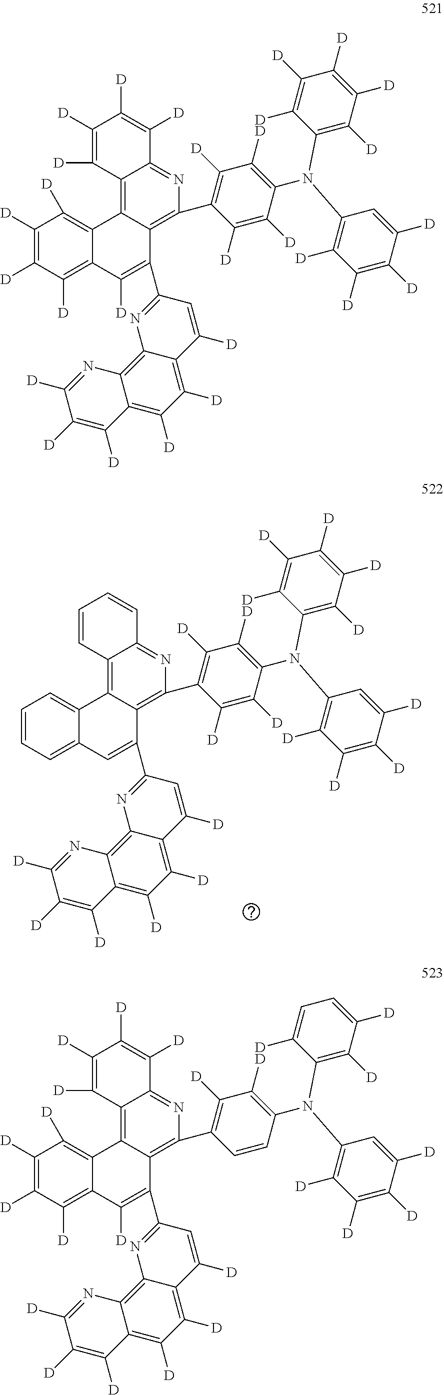

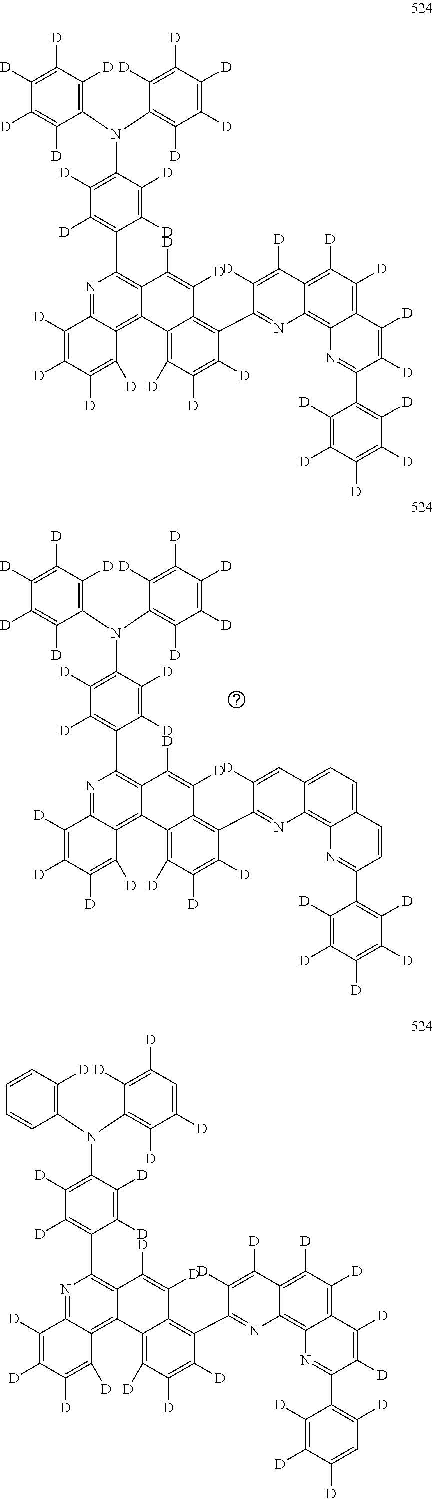

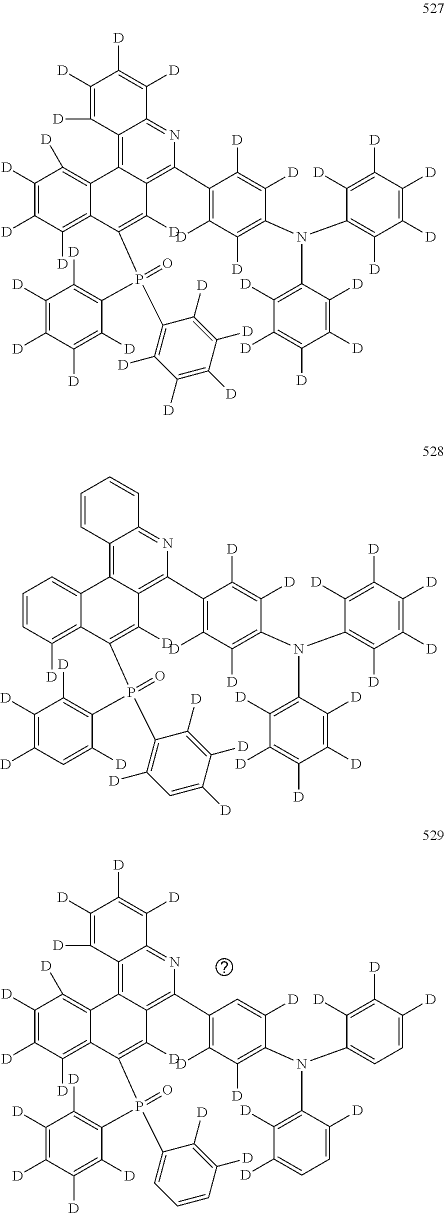

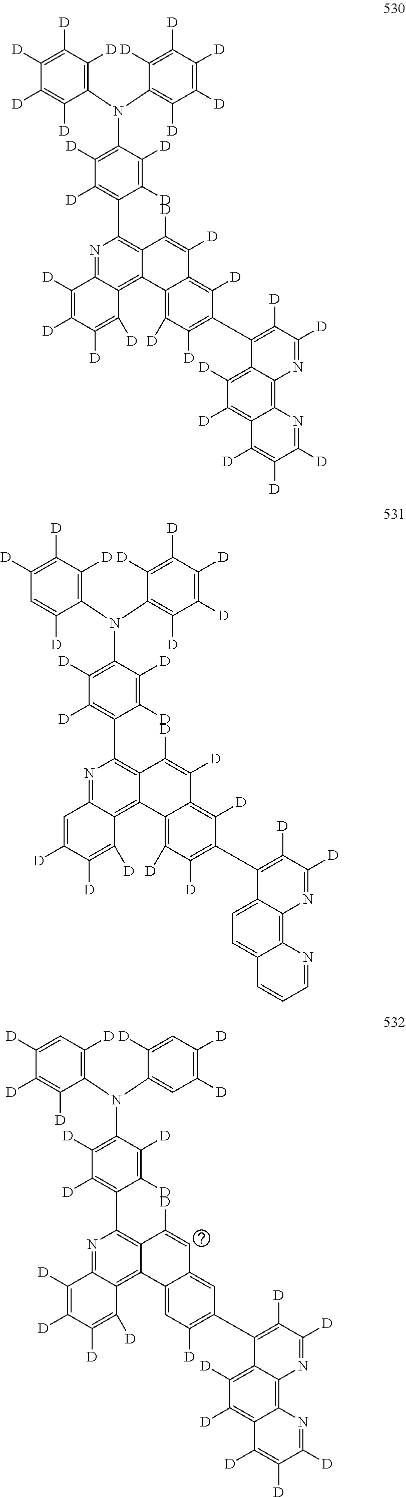

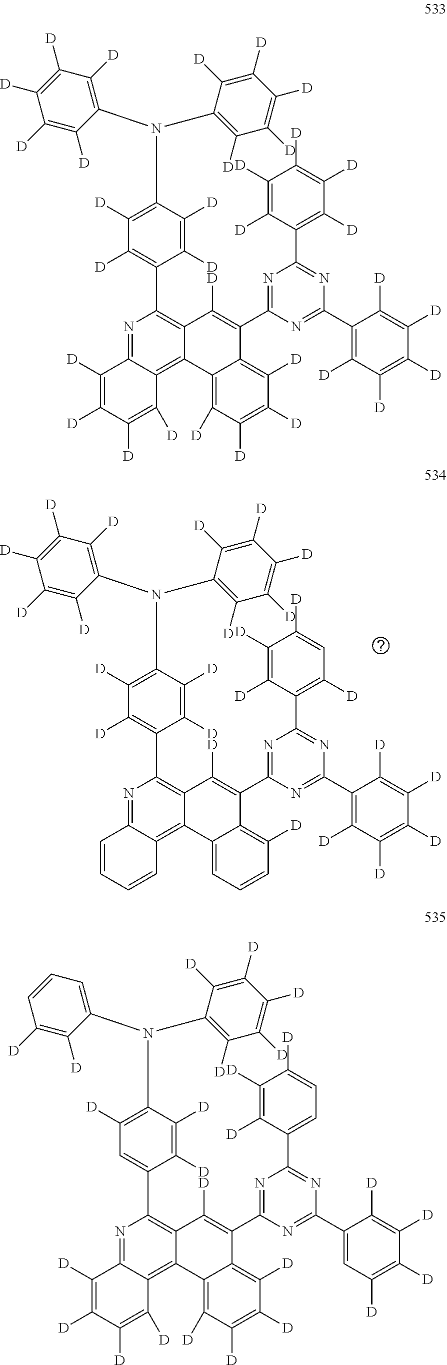

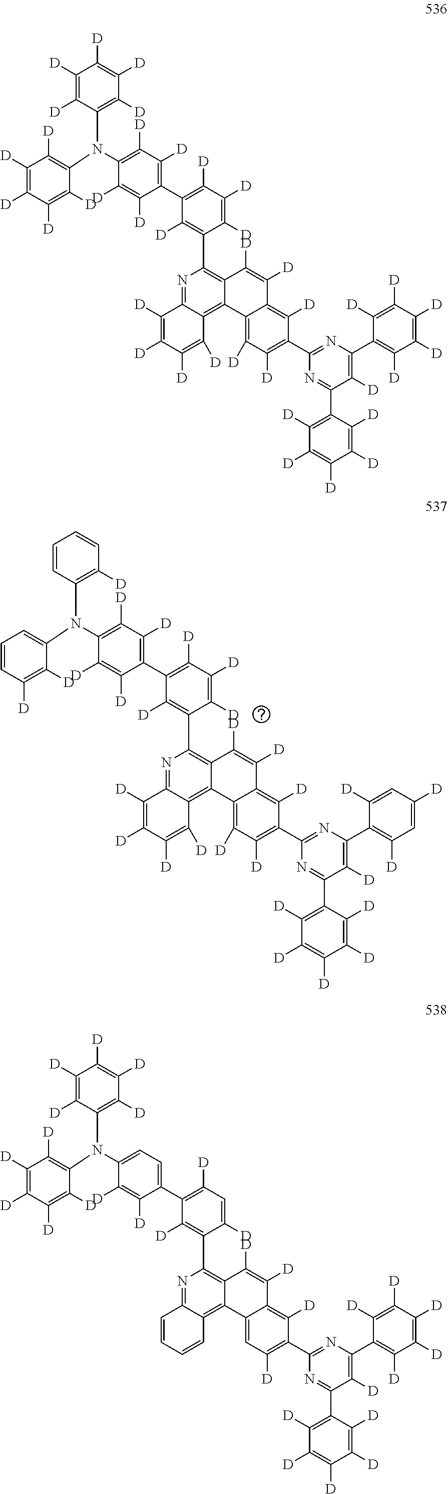

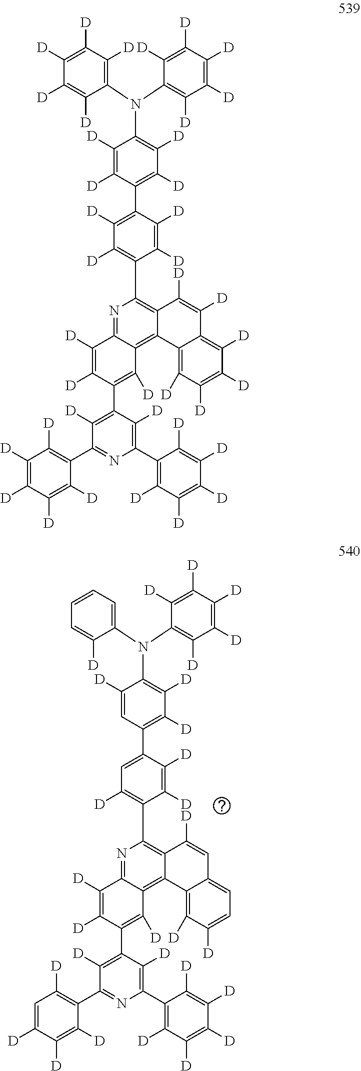

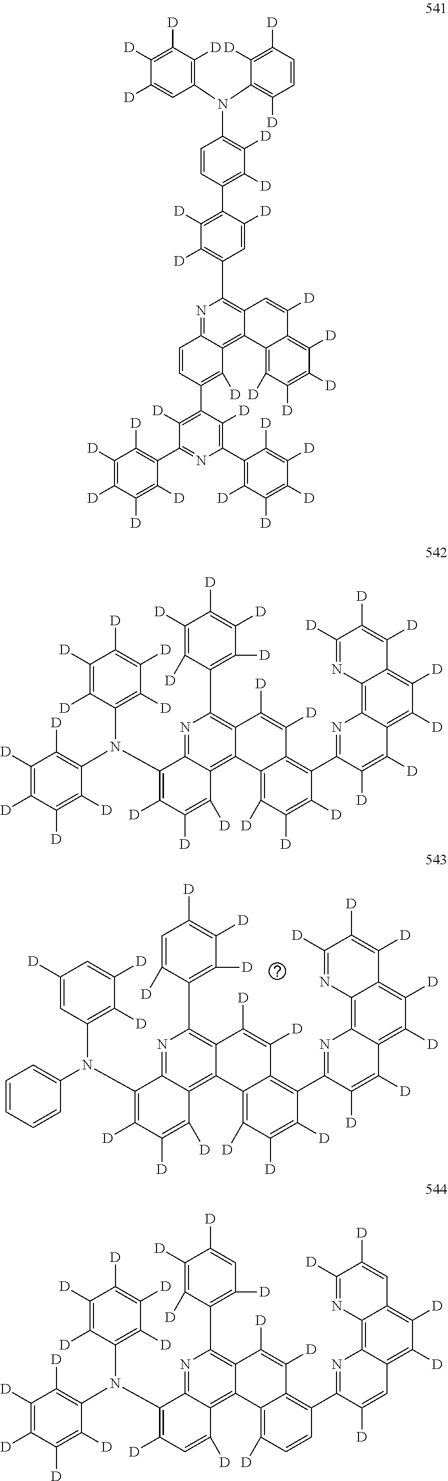

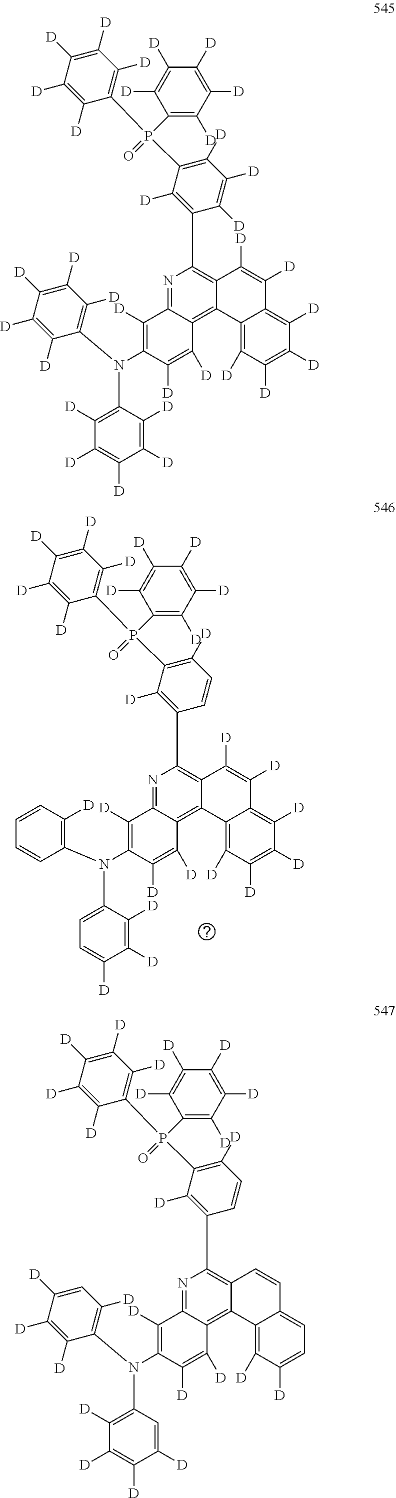

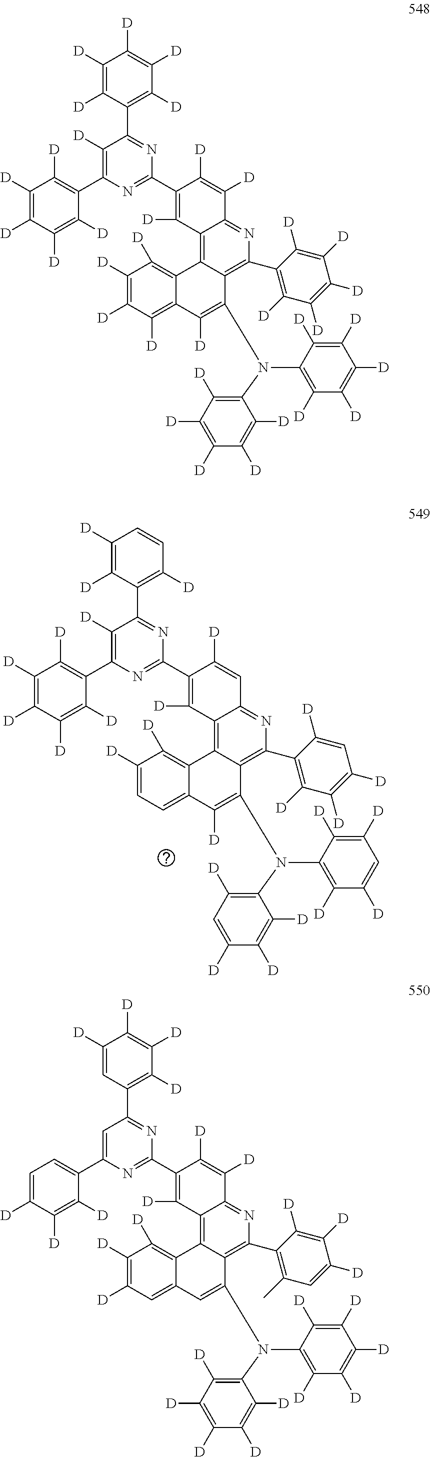

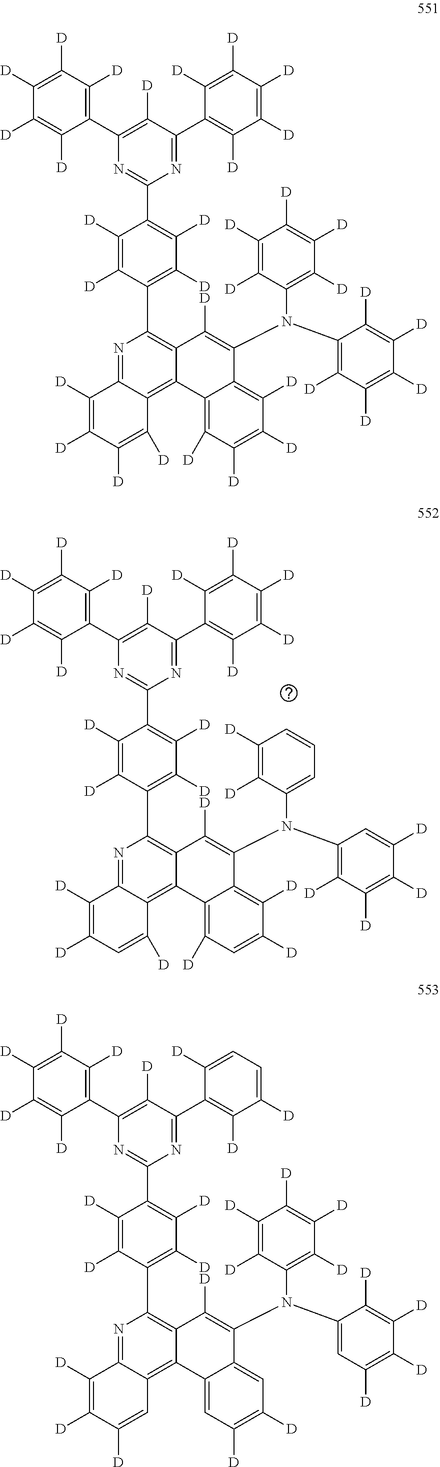

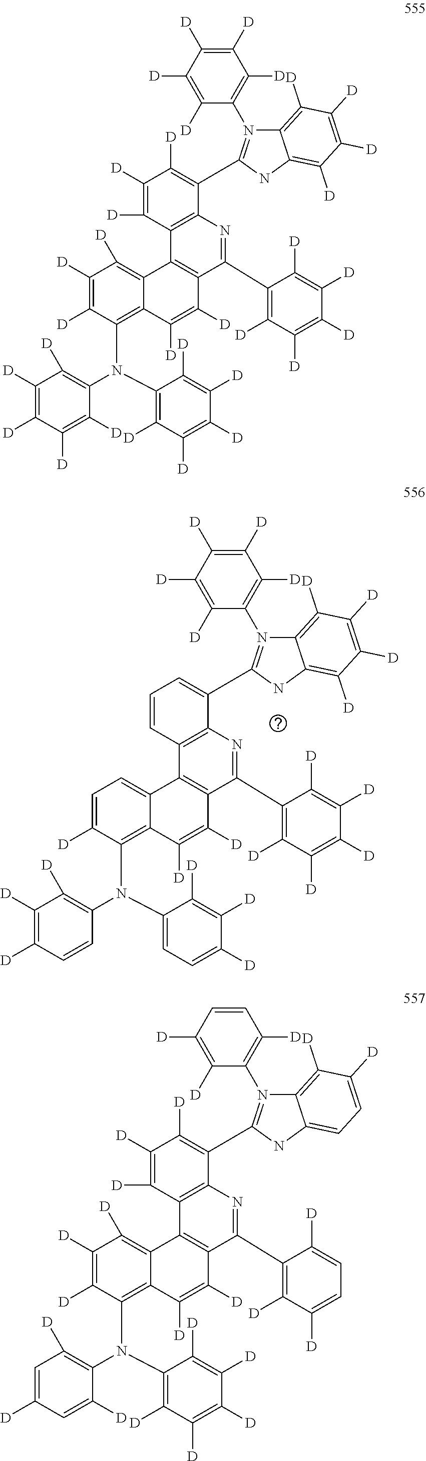

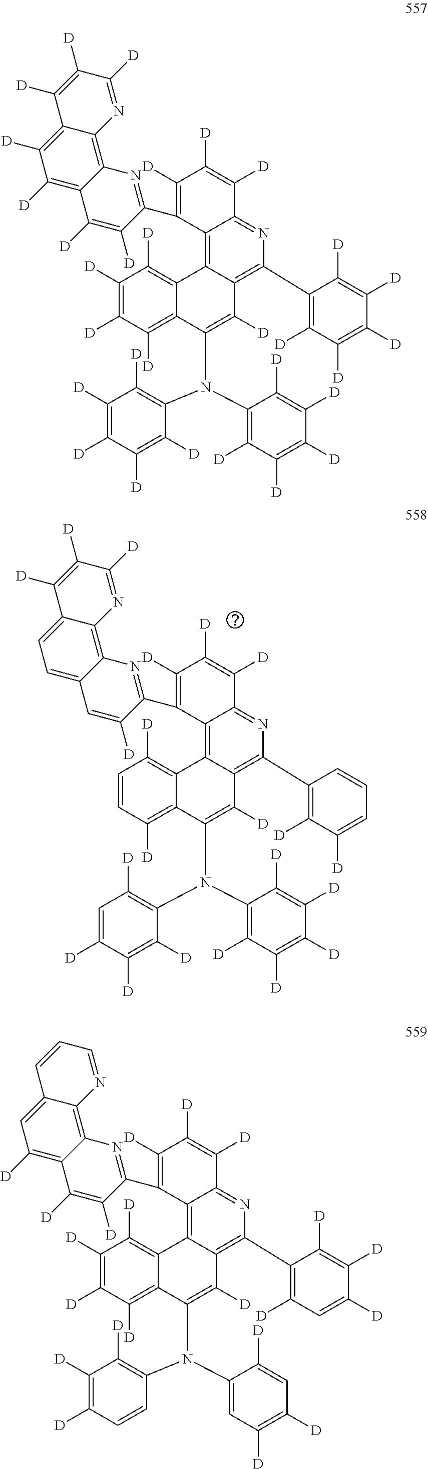

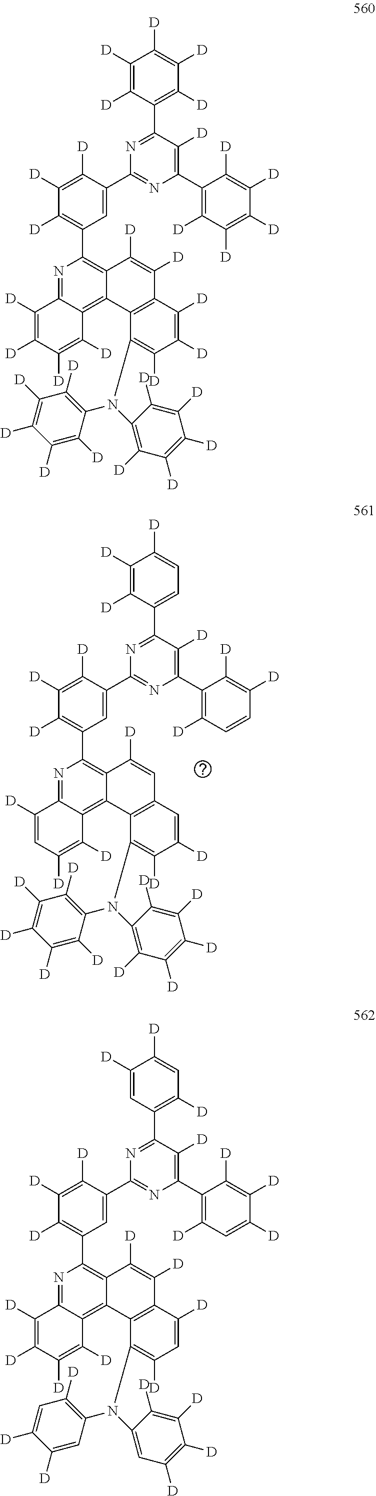

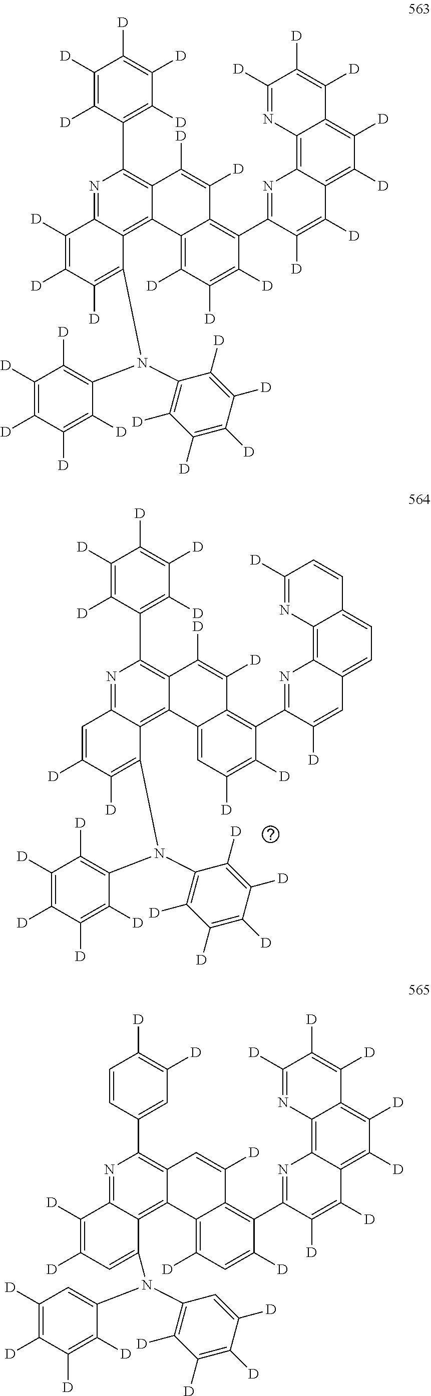

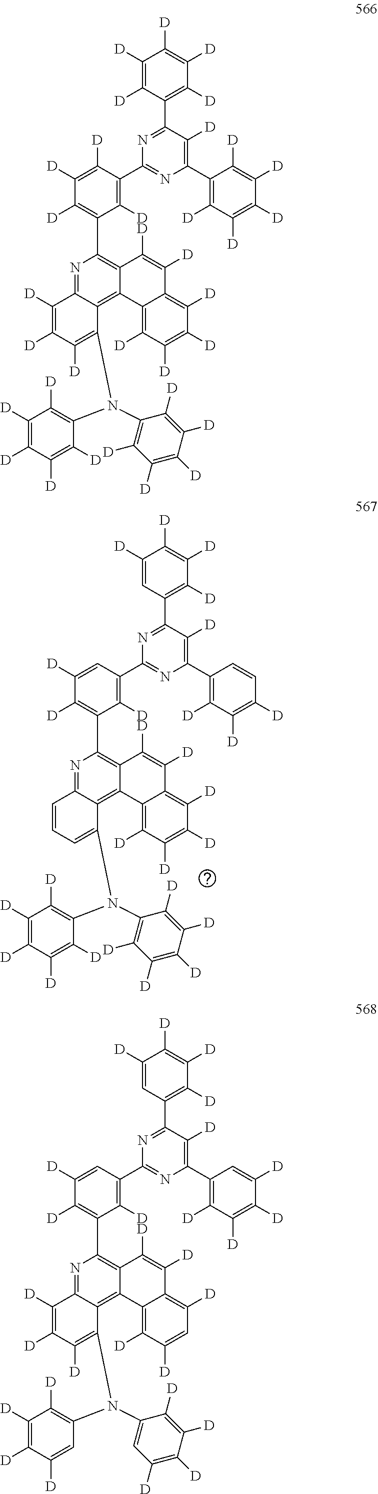

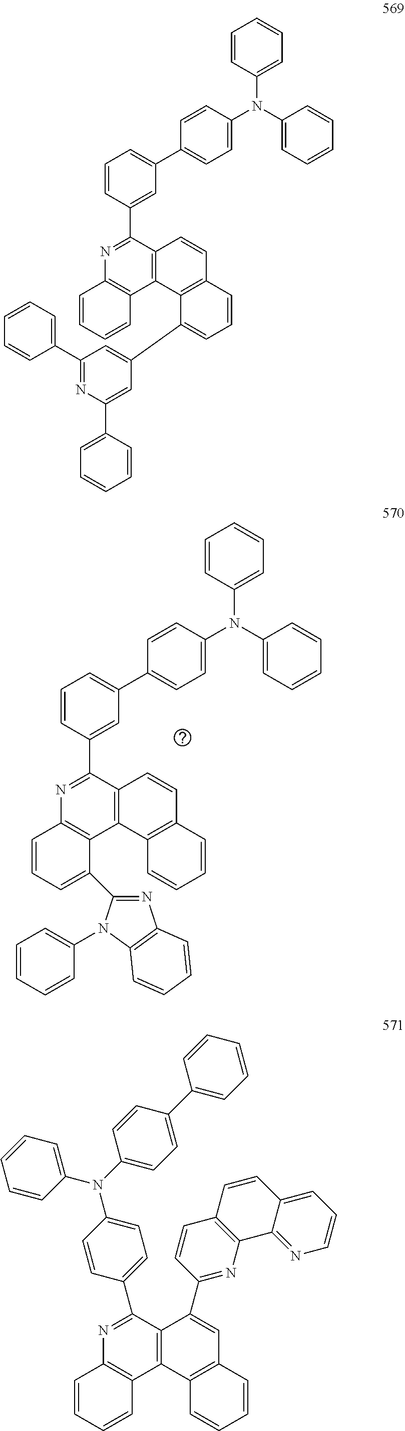 [SMILES: c1ccc(-c2cc(-c3cccc4ccc5c(-c6cccc(-c7ccc(N(c8ccccc8)c8ccccc8)cc7)c6)nc6ccccc6c5c34)cc(-c3ccccc3)n2)cc1 ; c1ccc(N(c2ccccc2)c2ccc(-c3cccc(-c4nc5cccc(-c6nc7ccccc7n6-c6ccccc6)c5c5c4ccc4ccccc45)c3)cc2)cc1 ; c1ccc(-c2ccc(N(c3ccccc3)c3ccc(-c4nc5ccccc5c5c4c(-c4ccc6ccc7cccnc7c6n4)cc4ccccc45)cc3)cc2)cc1]

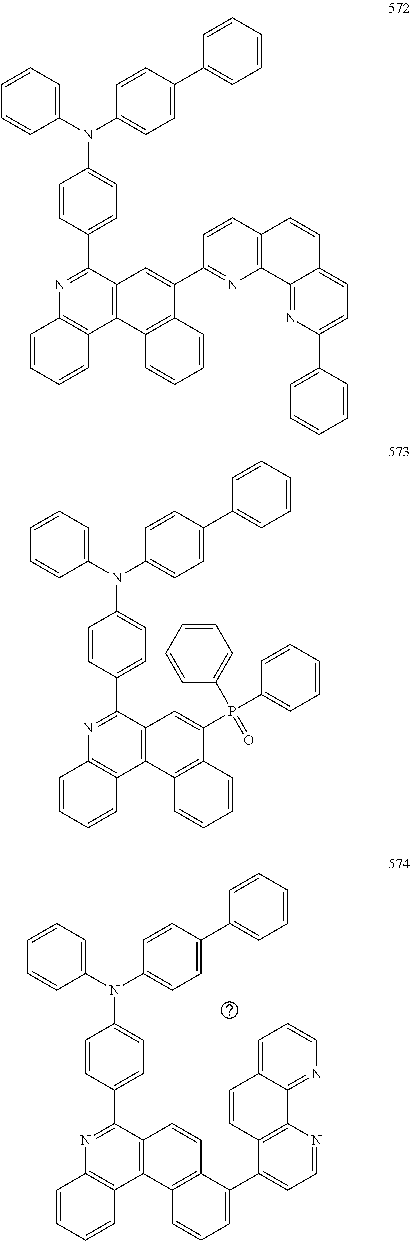 [SMILES: c1ccc(-c2ccc(N(c3ccccc3)c3ccc(-c4nc5ccccc5c5c4cc(-c4ccc6ccc7ccc(-c8ccccc8)nc7c6n4)c4ccccc45)cc3)cc2)cc1 ; O=P(c1ccccc1)(c1ccccc1)c1cc2c(-c3ccc(N(c4ccccc4)c4ccc(-c5ccccc5)cc4)cc3)nc3ccccc3c2c2ccccc12 ; c1ccc(-c2ccc(N(c3ccccc3)c3ccc(-c4nc5ccccc5c5c4ccc4c(-c6ccnc7c6ccc6cccnc67)cccc45)cc3)cc2)cc1]

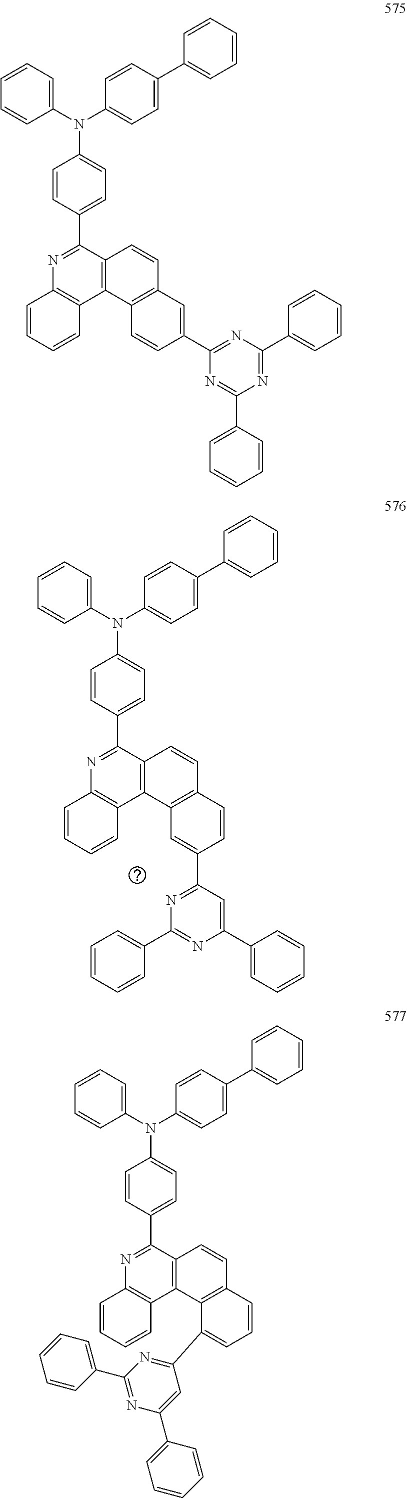

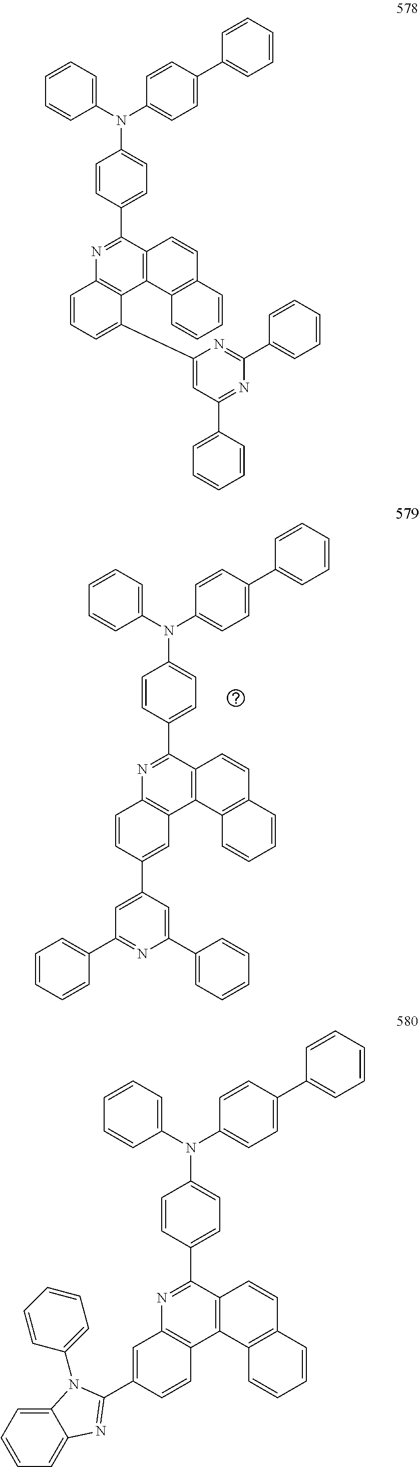 [SMILES: c1ccc(-c2ccc(N(c3ccccc3)c3ccc(-c4nc5cccc(-c6cc(-c7ccccc7)nc(-c7ccccc7)n6)c5c5c4ccc4ccccc45)cc3)cc2)cc1 ; c1ccc(-c2ccc(N(c3ccccc3)c3ccc(-c4nc5ccc(-c6cc(-c7ccccc7)nc(-c7ccccc7)c6)cc5c5c4ccc4ccccc45)cc3)cc2)cc1 ; c1ccc(-c2ccc(N(c3ccccc3)c3ccc(-c4nc5cc(-c6nc7ccccc7n6-c6ccccc6)ccc5c5c4ccc4ccccc45)cc3)cc2)cc1]

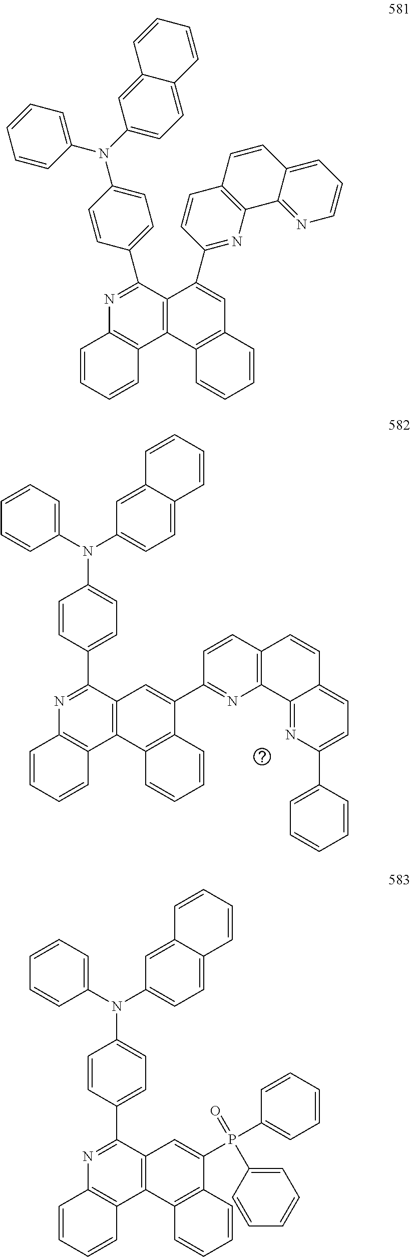 [SMILES: c1ccc(N(c2ccc(-c3nc4ccccc4c4c3c(-c3ccc5ccc6cccnc6c5n3)cc3ccccc34)cc2)c2ccc3ccccc3c2)cc1 ; c1ccc(-c2ccc3ccc4ccc(-c5cc6c(-c7ccc(N(c8ccccc8)c8ccc9ccccc9c8)cc7)nc7ccccc7c6c6ccccc56)nc4c3n2)cc1 ; O=P(c1ccccc1)(c1ccccc1)c1cc2c(-c3ccc(N(c4ccccc4)c4ccc5ccccc5c4)cc3)nc3ccccc3c2c2ccccc12]

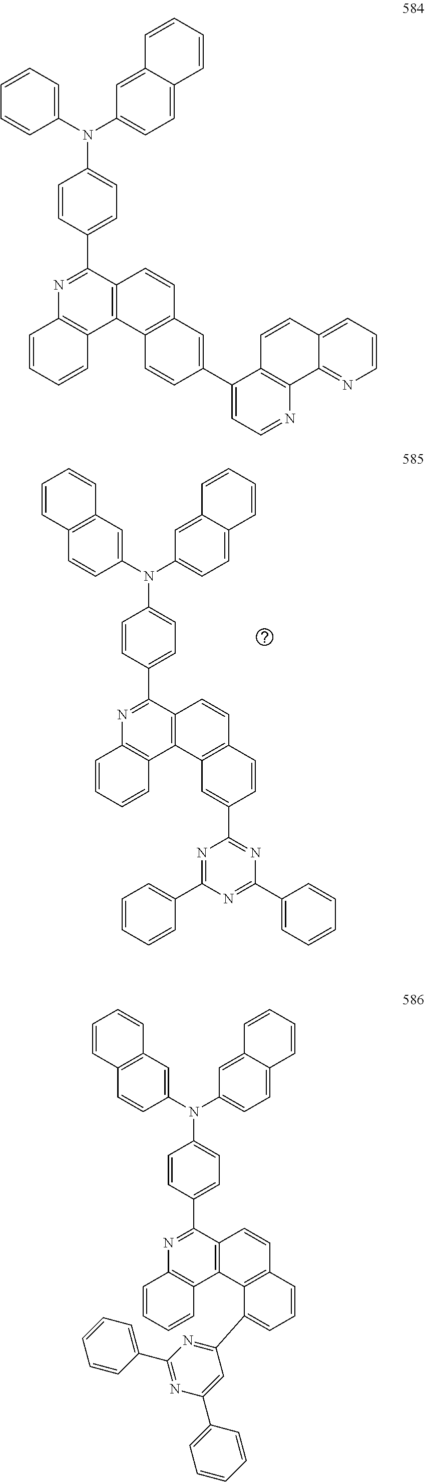 [SMILES: c1ccc(N(c2ccc(-c3nc4ccccc4c4c3ccc3cc(-c5ccnc6c5ccc5cccnc56)ccc34)cc2)c2ccc3ccccc3c2)cc1 ; c1ccc(-c2nc(-c3ccccc3)nc(-c3ccc4ccc5c(-c6ccc(N(c7ccc8ccccc8c7)c7ccc8ccccc8c7)cc6)nc6ccccc6c5c4c3)n2)cc1 ; c1ccc(-c2cc(-c3cccc4ccc5c(-c6ccc(N(c7ccc8ccccc8c7)c7ccc8ccccc8c7)cc6)nc6ccccc6c5c34)nc(-c3ccccc3)n2)cc1]

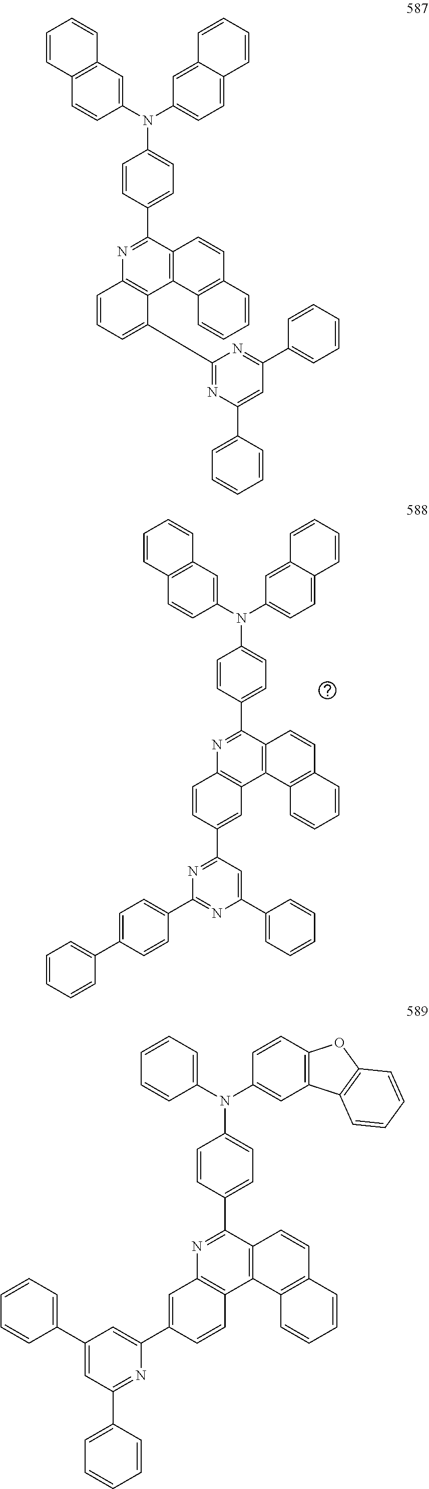 [SMILES: c1ccc(-c2cc(-c3ccccc3)nc(-c3cccc4nc(-c5ccc(N(c6ccc7ccccc7c6)c6ccc7ccccc7c6)cc5)c5ccc6ccccc6c5c34)n2)cc1 ; c1ccc(-c2ccc(-c3nc(-c4ccccc4)cc(-c4ccc5nc(-c6ccc(N(c7ccc8ccccc8c7)c7ccc8ccccc8c7)cc6)c6ccc7ccccc7c6c5c4)n3)cc2)cc1 ; c1ccc(-c2cc(-c3ccccc3)nc(-c3ccc4c(c3)nc(-c3ccc(N(c5ccccc5)c5ccc6oc7ccccc7c6c5)cc3)c3ccc5ccccc5c34)c2)cc1]

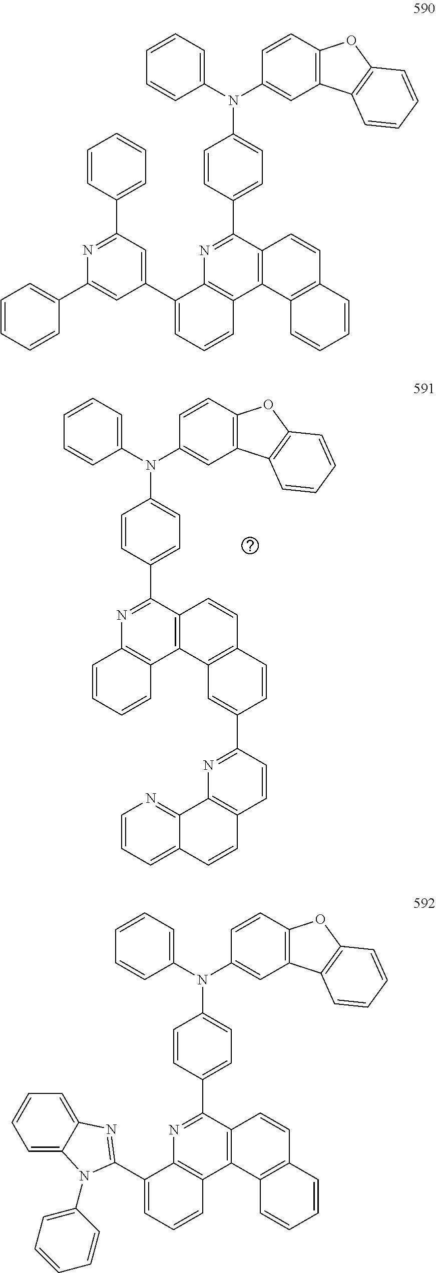 [SMILES: c1ccc(-c2cc(-c3cccc4c3nc(-c3ccc(N(c5ccccc5)c5ccc6oc7ccccc7c6c5)cc3)c3ccc5ccccc5c34)cc(-c3ccccc3)n2)cc1 ; c1ccc(N(c2ccc(-c3nc4ccccc4c4c3ccc3ccc(-c5ccc6ccc7cccnc7c6n5)cc34)cc2)c2ccc3oc4ccccc4c3c2)cc1 ; c1ccc(N(c2ccc(-c3nc4c(-c5nc6ccccc6n5-c5ccccc5)cccc4c4c3ccc3ccccc34)cc2)c2ccc3oc4ccccc4c3c2)cc1]

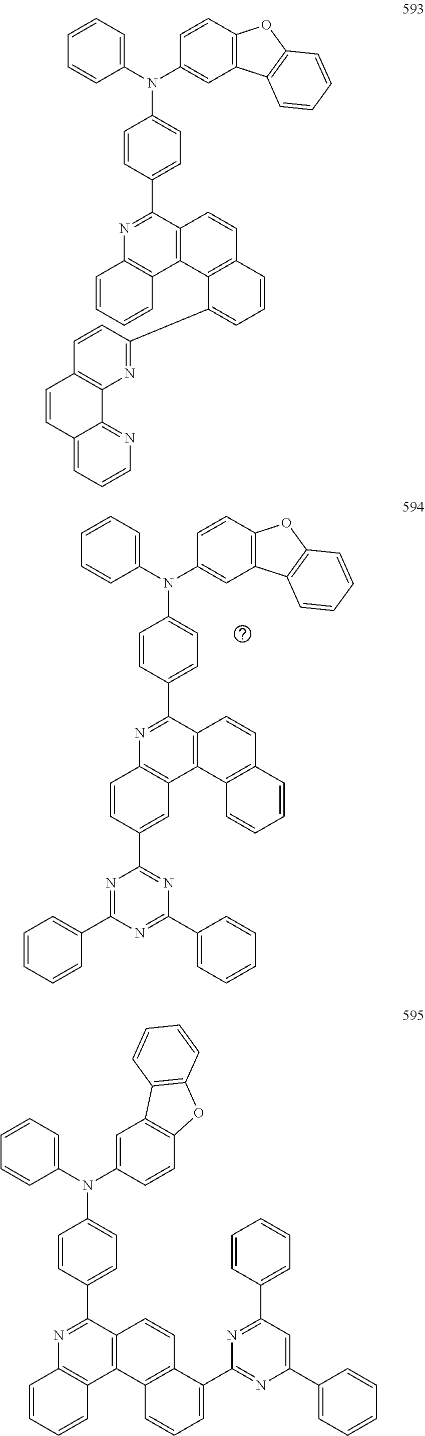

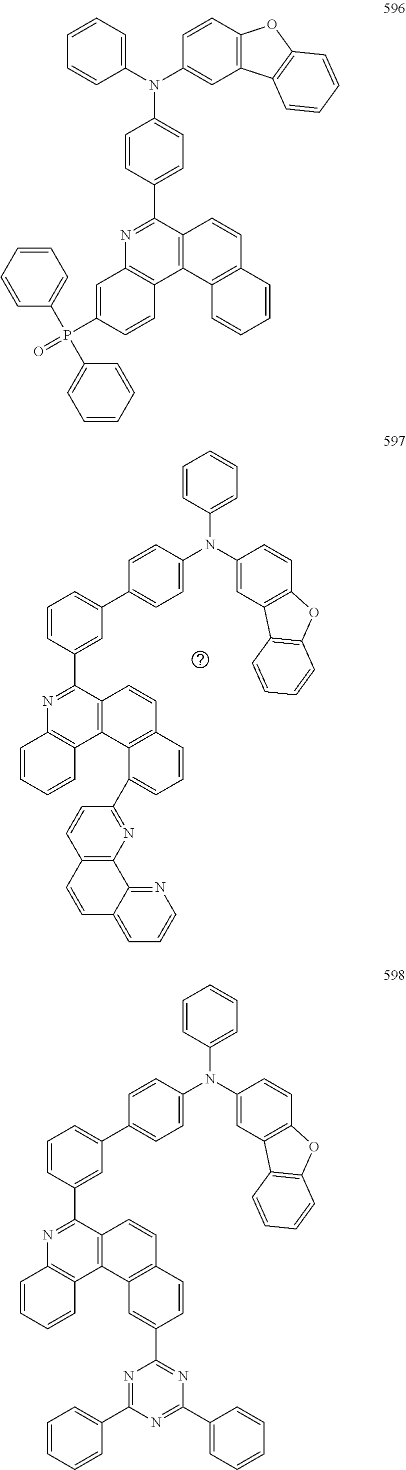 [SMILES: O=P(c1ccccc1)(c1ccccc1)c1ccc2c(c1)nc(-c1ccc(N(c3ccccc3)c3ccc4oc5ccccc5c4c3)cc1)c1ccc3ccccc3c12 ; c1ccc(N(c2ccc(-c3cccc(-c4nc5ccccc5c5c4ccc4cccc(-c6ccc7ccc8cccnc8c7n6)c45)c3)cc2)c2ccc3oc4ccccc4c3c2)cc1 ; c1ccc(-c2nc(-c3ccccc3)nc(-c3ccc4ccc5c(-c6cccc(-c7ccc(N(c8ccccc8)c8ccc9oc%10ccccc%10c9c8)cc7)c6)nc6ccccc6c5c4c3)n2)cc1]

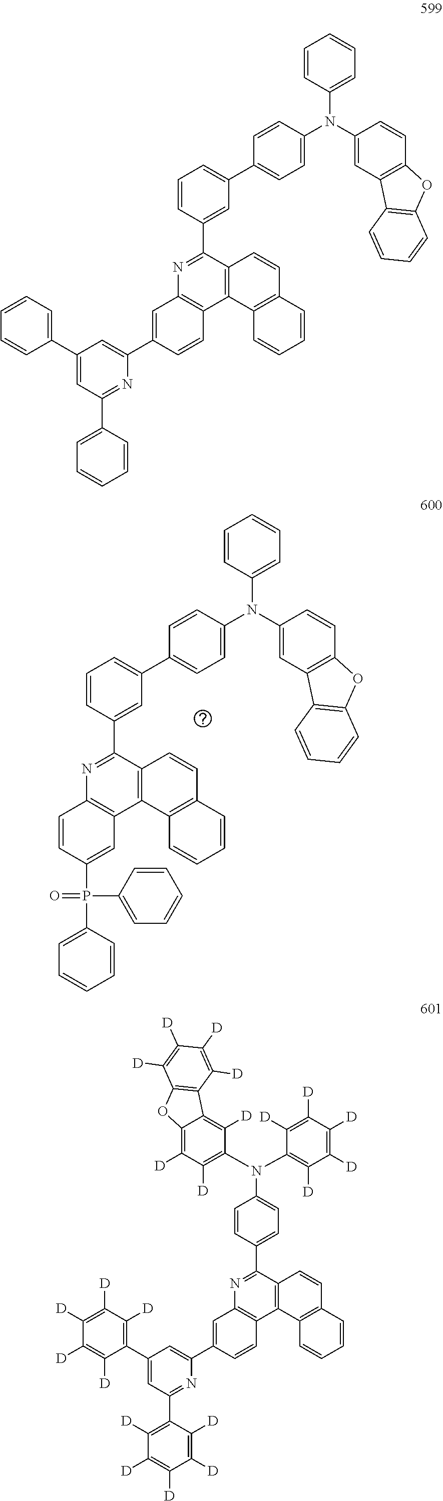 [SMILES: c1ccc(-c2cc(-c3ccccc3)nc(-c3ccc4c(c3)nc(-c3cccc(-c5ccc(N(c6ccccc6)c6ccc7oc8ccccc8c7c6)cc5)c3)c3ccc5ccccc5c34)c2)cc1 ; O=P(c1ccccc1)(c1ccccc1)c1ccc2nc(-c3cccc(-c4ccc(N(c5ccccc5)c5ccc6oc7ccccc7c6c5)cc4)c3)c3ccc4ccccc4c3c2c1 ; [2H]c1c([2H])c([2H])c(-c2cc(-c3ccc4c(c3)nc(-c3ccc(N(c5c([2H])c([2H])c([2H])c([2H])c5[2H])c5c([2H])c([2H])c6oc7c([2H])c([2H])c([2H])c([2H])c7c6c5[2H])cc3)c3ccc5ccccc5c34)nc(-c3c([2H])c([2H])c([2H])c([2H])c3[2H])c2)c([2H])c1[2H]]

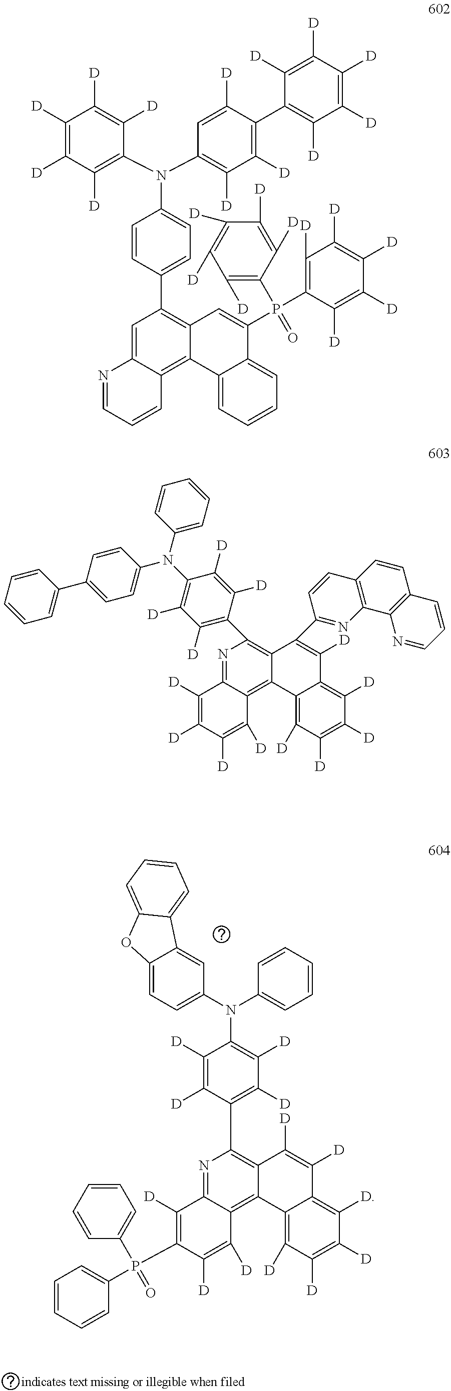

9. An organic light emitting device comprising: a first electrode; a second electrode provided to face the first electrode; and an organic material layer having one or more layers provided between the first electrode and the second electrode, wherein one or more layers of the organic material layer comprise the heterocyclic compound of claim 1.

10. The organic light emitting device of claim 9, wherein the organic material layer comprises a light emitting layer, and the light emitting layer comprises the heterocyclic compound represented by Chemical Formula 1.

11. The organic light emitting device of claim 9, wherein the organic material layer comprises a light emitting layer, the light emitting layer comprises a host material, and the host material comprises the heterocyclic compound represented by Chemical Formula 1.

12. The organic light emitting device of claim 9, wherein the organic material layer comprises an electron injection layer or an electron transport layer, and the electron injection layer or the electron transport layer comprises the heterocyclic compound represented by Chemical Formula 1.

13. The organic light emitting device of claim 9, wherein the organic material layer comprises an electron blocking layer or a hole blocking layer, and the electron blocking layer or the hole blocking layer comprises the heterocyclic compound represented by Chemical Formula 1.

14. The organic light emitting device of claim 9, further comprising one or two or more layers selected from the group consisting of a light emitting layer, a hole injection layer, a hole transport layer, an electron injection layer, an electron transport layer, an electron blocking layer and a hole blocking layer.

15. The organic light emitting device of claim 9, wherein the organic light emitting device comprises a first electrode, a first stack provided on the first electrode and comprising a first light emitting layer, a charge generation layer provided on the first stack, a second stack provided on the charge generation layer and comprising a second light emitting layer, and a second electrode provided on the second stack.

16. The organic light emitting device of claim 15, wherein the charge generation layer comprises the heterocyclic compound.
